SEQUENCE LISTING (43 sequences)

SEQ ID NO: 1 - protein, 9 aa, Homo sapiens
KVLEYVIKV

SEQ ID NO: 2 - protein, 8 aa, Homo sapiens
VLEYVIKV

SEQ ID NO: 3 - protein, 8 aa, Homo sapiens
KVLEYVIK

SEQ ID NO: 4 - DNA, 810 bp, synthetic construct
  source 1..810: mol_type = other DNA
atggaaaaaatgctcgagtgcgccttcatcgtgctttggctgcagctcggatggctgagcggagaggatcaagtgacacagtctcccgaggctctgaggctgcaagagggcgaaagcagctccctgaattgcagctacaccgtgtctggcctgaggggcctgttttggtacagacaagaccctggcaagggacccgagttcctgttcacactgtactctgccggcgaagaaaaagagaaagagcgcctgaaagcaaccctgaccaagaaagagagcttcctgcacatcacagcccctaagccagaggacagcgctacttacctgtgtgccgtttcatacggccagaatttcgtttttggtcccggaaccagattgtccgtgctgccctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 5 - protein, 270 aa, synthetic construct
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVSYGQNFVFGPGTRLSVLPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 6 - DNA, 927 bp, synthetic construct
  source 1..927: mol_type = other DNA
atgggacccaggctcctcttctgggcactgctttgtctcctcggaacaggcccagtggaggctggagtcacacaaagtcccacacacctgatcaaaacgagaggacagcaagcgactctgagatgctctcctatctctgggcacaccagtgtgtactggtaccaacaggccctgggtctgggcctccagttcctcctttggtatgacgagggtgaagagagaaacagaggaaacttccctcctagattttcaggtcgccagttccctaattatagctctgagctgaatgtgaacgccttggagctggaggactcggccctgtatctctgtgcttcctcacttgggcaattgaacacagaggcattctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 7 - protein, 309 aa, synthetic construct
MGPRLLFWALLCLLGTGPVEAGVTQSPTHLIKTRGQQATLRCSPISGHTSVYWYQQALGLGLQFLLWYDEGEERNRGNFPPRFSGRQFPNYSSELNVNALELEDSALYLCASSLGQLNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 8 - DNA, 810 bp, synthetic construct
  source 1..810: mol_type = other DNA
atggaaaaaatgctcgagtgcgccttcatcgtgctttggctgcagctcggatggctgagcggagaggatcaagtgacacagtctcccgaggctctgaggctgcaagagggcgaaagcagctccctgaattgcagctacaccgtgtctggcctgaggggcctgttttggtacagacaagaccctggcaagggacccgagttcctgttcacactgtactctgccggcgaagaaaaagagaaagagcgcctgaaagcaaccctgaccaagaaagagagcttcctgcacatcacagcccctaagccagaggacagcgctacttacctgtgtgccgtttcatacggccagaatttcgtttttggtcccggaaccagattgtccgtgctgccctacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagcagc

SEQ ID NO: 9 - protein, 270 aa, synthetic construct
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVSYGQNFVFGPGTRLSVLPYIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 10 - DNA, 936 bp, synthetic construct
  source 1..936: mol_type = other DNA
atgggacccaggctcctcttctgggcactgctttgtctcctcggaacaggcccagtggaggctggagtcacacaaagtcccacacacctgatcaaaacgagaggacagcaagcgactctgagatgctctcctatctctgggcacaccagtgtgtactggtaccaacaggccctgggtctgggcctccagttcctcctttggtatgacgagggtgaagagagaaacagaggaaacttccctcctagattttcaggtcgccagttccctaattatagctctgagctgaatgtgaacgccttggagctggaggactcggccctgtatctctgtgcttcctcacttgggcaattgaacacagaggcattctttggacaaggcaccagactcacagttgtagaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggc

SEQ ID NO: 11 - protein, 312 aa, synthetic construct
MGPRLLFWALLCLLGTGPVEAGVTQSPTHLIKTRGQQATLRCSPISGHTSVYWYQQALGLGLQFLLWYDEGEERNRGNFPPRFSGRQFPNYSSELNVNALELEDSALYLCASSLGQLNTEAFFGQGTRLTVVEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSG

SEQ ID NO: 12 - DNA, 1827 bp, synthetic construct
  source 1..1827: mol_type = other DNA
atgggacccaggctcctcttctgggcactgctttgtctcctcggaacaggcccagtggaggctggagtcacacaaagtcccacacacctgatcaaaacgagaggacagcaagcgactctgagatgctctcctatctctgggcacaccagtgtgtactggtaccaacaggccctgggtctgggcctccagttcctcctttggtatgacgagggtgaagagagaaacagaggaaacttccctcctagattttcaggtcgccagttccctaattatagctctgagctgaatgtgaacgccttggagctggaggactcggccctgtatctctgtgcttcctcacttgggcaattgaacacagaggcattctttggacaaggcaccagactcacagttgtagaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggtgcgacaaactttagcctgttgaaacaagccggcgacgttgaagagaaccccggacctatggaaaaaatgctcgagtgcgccttcatcgtgctttggctgcagctcggatggctgagcggagaggatcaagtgacacagtctcccgaggctctgaggctgcaagagggcgaaagcagctccctgaattgcagctacaccgtgtctggcctgaggggcctgttttggtacagacaagaccctggcaagggacccgagttcctgttcacactgtactctgccggcgaagaaaaagagaaagagcgcctgaaagcaaccctgaccaagaaagagagcttcctgcacatcacagcccctaagccagaggacagcgctacttacctgtgtgccgtttcatacggccagaatttcgtttttggtcccggaaccagattgtccgtgctgccctacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagcagc

SEQ ID NO: 13 - protein, 609 aa, synthetic construct
MGPRLLFWALLCLLGTGPVEAGVTQSPTHLIKTRGQQATLRCSPISGHTSVYWYQQALGLGLQFLLWYDEGEERNRGNFPPRFSGRQFPNYSSELNVNALELEDSALYLCASSLGQLNTEAFFGQGTRLTVVEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSGRAKRSGSGATNFSLLKQAGDVEENPGPMEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVSYGQNFVFGPGTRLSVLPYIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 14 - DNA, 930 bp, Homo sapiens
  source 1..930: mol_type = genomic DNA
atgtctcttgagcagaggagtctgcactgcaagcctgaggaagcccttgaggcccaacaagaggccctgggcctggtgtgtgtgcaggctgccacctcctcctcctctcctctggtcctgggcaccctggaggaggtgcccactgctgggtcaacagatcctccccagagtcctcagggagcctccgcctttcccactaccatcaacttcactcgacagaggcaacccagtgagggttccagcagccgtgaagaggaggggccaagcacctcttgtatcctggagtccttgttccgagcagtaatcactaagaaggtggctgatttggttggttttctgctcctcaaatatcgagccagggagccagtcacaaaggcagaaatgctggagagtgtcatcaaaaattacaagcactgttttcctgagatcttcggcaaagcctctgagtccttgcagctggtctttggcattgacgtgaaggaagcagaccccaccggccactcctatgtccttgtcacctgcctaggtctctcctatgatggcctgctgggtgataatcagatcatgcccaagacaggcttcctgataattgtcctggtcatgattgcaatggagggcggccatgctcctgaggaggaaatctgggaggagctgagtgtgatggaggtgtatgatgggagggagcacagtgcctatggggagcccaggaagctgctcacccaagatttggtgcaggaaaagtacctggagtaccggcaggtgccggacagtgatcccgcacgctatgagttcctgtggggtccaagggccctcgctgaaaccagctatgtgaaagtccttgagtatgtgatcaaggtcagtgcaagagttcgctttttcttcccatccctgcgtgaagcagctttgagagaggaggaagagggagtctga

SEQ ID NO: 15 - protein, 309 aa, Homo sapiens
MSLEQRSLHCKPEEALEAQQEALGLVCVQAATSSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGV

SEQ ID NO: 16 - DNA, 1098 bp, Homo sapiens
  source 1..1098: mol_type = genomic DNA
atggccgtcatggcgccccgaaccctcgtcctgctactctcgggggctctggccctgacccagacctgggcgggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgtcttcccagcccaccatccccatcgtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtgtga

SEQ ID NO: 17 - protein, 365 aa, Homo sapiens [sequence omitted]

SEQ ID NO: 18 - DNA, 9683 bp, synthetic construct
  source 1..9683: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacaggcgcgccagagatccagtttggacctgcaggtgaaagaccccacctgtaggtttggcaagttagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagcgaaccatcagatgtttccagggtgccccaaggacctgaaatgaccctgtgccttatttgaactaaccaatcagtttgcttcttgcttctgtttgtgtgcttctgctccctgagctcaataaaagagcccacaacccctcacttggtgggccagtcctctgatagactgtgtcccctggatacccgtacggtaccgctagcgccaccatgggcaccagcctcctctgctggatggccctgtgtctcctgggggcagatcacgcagatactggagtctcccaggaccccagacacaagatcacaaagaggggacagaatgttactttcaggtgtgatccaatttctgaacacaaccgcctttattggtaccgccagaccctggggcagggcccagagtttctgacttacttccagaatgaagctcaacttgaaaaatcaaggctgctcagtgatcggttctctgcagagaggcctaagggatctttctccaccttggagatccagcgcacagagcagggggactctgccatgtatctctgtgccagcagccgcactgctggagatactcagtattttggcccaggcacccggctgacagtgctcgaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggtgcgacaaactttagcctgttgaaacaagccggcgacgttgaagagaaccccggacctatggaaaccctcttgggcctgcttatcctttggctgcagctgcaatgggtgagcagcaaacaggaggtgactcagattcctgcagctctgagtgtcccagaaggagaaaacttggttctcaactgcagtttcactgatagcgctatttacaacctccagtggtttaggcaggaccctgggaaaggcctcacatctctgttgcttattcagtcaagtcagagagagcaaacaagtggacgccttaatgcctctctggataaatcatcaggacgcagtactctttacattgcagcttctcagcctggtgattcagccacctacctgtgcgctgtgaggggtggtacctcaggaacctacaaatacatctttggaacaggcaccaggctgaaggttcttgcaaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagcagcagggctaagaggtccggcagcggagccaccaatttttccctgctgaaacaggctggtgacgtggaagaaaaccctggccccatggcgctgcccgtcaccgcgctgctgctgcccctggcgctgctgttacacgccgctcggccagagcttcccacccagggcacattctccaacgtgtccaccaatgtgtcgggaggcggcggatcgtcccagttcagagtgtcccctctggaccgcacctggaacctgggcgagaccgtggagctgaaatgtcaggtcctgctgagcaacccgacctccgggtgcagttggctgttccagccgcgtggtgctgccgcaagccctacgttcctgctttacctgagccagaacaagcccaaggcggccgagggcctggacacccagagattctccggcaagcgcctgggggacacattcgtgcttactttgagcgatttccgcagagagaacgagggctactatttctgttcggcgctgagcaattccatcatgtatttcagccactttgtgccagtgttcctgcctgccaagcctaccacaacaccagctccccgtcccccgactccggcgcctaccatcgcgagtcaaccgttgagcctgaggcctgaggcttgtcggcccgctgcggggggtgccgtccacaccaggggcctcgactttgcgtgcgacatctatatttgggcgcctctggcgggtacctgcggggtgctgctgctgtcattggtgattaccctgtactgcaatcaccgcaaccgccggcgggtctgtaagtgcccacggcctgtggtcaagtccggtgacaaaccgtcgctctcggctcgctacgtgcgcgctaagcgcagcggttccggggccaccaacttttcattgctgaagcaggccggtgatgtggaggagaatccagggcccatgcgccccaggctttggctccttcttgctgctcagctcactgtcttgcatggcaactccgttctgcagcagactcccgcctacatcaaggtgcagacgaacaagatggtgatgctgtcatgcgaggccaagatctctctttcaaatatgagaatttattggctacgacagcgccaggccccctccagcgacagccaccacgagttcctggcgctttgggattctgctaaaggcaccatccatggagaggaggtggaacaggagaagatagctgtcttccgcgacgcatcccgcttcatcctgaacctgaccagcgtgaagccggaggacagcggcatctacttctgtatgatcgttggctcccccgagctgaccttcggcaaaggcacccagctgtccgtggtggacttcctgcccaccacagcccagccaaccaagaaatccaccctcaagaagcgcgtgtgccgactgccccgccctgaaacccagaagggccctctgtgctcccccatcacccttggactgctggtggcgggagtcctggtgctgctcgtatctctgggtgtcgccatccacctgtgctgccgccgccgccgcgcccgcctgaggtttatgaaacagttttacaagtgataaatcgatagatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgagatcctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggcccgggttaattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 19 - DNA, 8789 bp, synthetic construct
  source 1..8789: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacaggcgcgccagagatccagtttggacctgcaggtgaaagaccccacctgtaggtttggcaagttagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagcgaaccatcagatgtttccagggtgccccaaggacctgaaatgaccctgtgccttatttgaactaaccaatcagtttgcttcttgcttctgtttgtgtgcttctgctccctgagctcaataaaagagcccacaacccctcacttggtgggccagtcctctgatagactgtgtcccctggatacccgtacggtaccgctagcgccaccatgggctcctggaccctctgctgtgtgtccctttgcatcctggttgcaaagcacacagatgctggagttatccagtcaccccggcacgaggtgacagagatgggacaagaagtgactctgagatgtaaaccaatttcaggacatgactaccttttctggtacagacagaccatgatgcggggactggagttgctcatttactttaacaacaacgttcctattgatgattcagggatgcccgaggatcgcttctcagctaagatgcctaatgcatcattctccactctgaagatccagccctcagaacccagggactcagctgtgtacttctgtgccagcagttttctcggctggaatgaaaaactgttctttggcagtggaacccagctctctgtcttggaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggtgcgacaaactttagcctgttgaaacaagccggcgacgttgaagagaaccccggacctatggtcctgaaattctccgtgtccattctttggattcagttggcatgggtgagcacccagctgctggagcagagccctcagtttcttagcatccaagagggagaaaatctcactgtgtactgcaactcctcaagtgttttctccagccttcaatggtacagacaggagcctggggaaggtcctgtcctcctggtgacagttgttactggtggagaagtgaagaagctgaagagacttacctttcagtttggtgatgcaagaaaggacagttctctccacatcactgcagcccagcctggtgatacaggcctctacctctgtgcaggagatgaaagtattagctatggaaagctgacatttggacaagggaccatcttgactgtccatccaaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagcagcagggctaagaggtccggcagcggagccaccaatttttccctgctgaaacaggctggtgacgtggaagaaaaccctggccccatgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagtagtgataaatcgatagatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgagatcctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggcccgggttaattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 20 - DNA, 8040 bp, synthetic construct
  source 1..8040: mol_type = other DNA
gctagctggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacatactatcaacaggttgaactgctgatctgtacagtagaattggtaaagagagttgtgtaaaatattgagttcgcacatcttgttgtctgattattgatttttggcgaaaccatttgatcatatgacaagatgtgtatctaccttaacttaatgattttgataaaaatcattaggtaccaattacattgcttgcaattaaccctttaacggttataaggatctagatgagatagaaagatttggttttcggatttgtgttacataagatgcctaaaataaaaattgagattcaattttttttaaacttttttttaattggtggtaagaatattccctctacctgtttgagagtaatgaaattgtagtatgatttttcaacaaactaaaaaaacaacataaatctcacataataactttatttcaatcacacaattgaataccaataggttgacagtacttaccagcctgcaggtgaaagaccccacctgtaggtttggcaagttagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagcgaaccatcagatgtttccagggtgccccaaggacctgaaatgaccctgtgccttatttgaactaaccaatcagtttgcttcttgcttctgtttgtgtgcttctgctccctgagctcaataaaagagcccacaacccctcacttggtgggccagtcctctgatagactgtgtcccctggatacccgtacggtaccgctagcgccaccatgggacccaggctcctcttctgggcactgctttgtctcctcggaacaggcccagtggaggctggagtcacacaaagtcccacacacctgatcaaaacgagaggacagcaagcgactctgagatgctctcctatctctgggcacaccagtgtgtactggtaccaacaggccctgggtctgggcctccagttcctcctttggtatgacgagggtgaagagagaaacagaggaaacttccctcctagattttcaggtcgccagttccctaattatagctctgagctgaatgtgaacgccttggagctggaggactcggccctgtatctctgtgcttcctcacttgggcaattgaacacagaggcattctttggacaaggcaccagactcacagttgtagaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggtgcgacaaactttagcctgttgaaacaagccggcgacgttgaagagaaccccggacctatggaaaaaatgctcgagtgcgccttcatcgtgctttggctgcagctcggatggctgagcggagaggatcaagtgacacagtctcccgaggctctgaggctgcaagagggcgaaagcagctccctgaattgcagctacaccgtgtctggcctgaggggcctgttttggtacagacaagaccctggcaagggacccgagttcctgttcacactgtactctgccggcgaagaaaaagagaaagagcgcctgaaagcaaccctgaccaagaaagagagcttcctgcacatcacagcccctaagccagaggacagcgctacttacctgtgtgccgtttcatacggccagaatttcgtttttggtcccggaaccagattgtccgtgctgccctacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagcagcagggctaagaggtccggcagcggagccaccaatttttccctgctgaaacaggctggtgacgtggaagaaaaccctggccccatggcgctgcccgtcaccgcgctgctgctgcccctggcgctgctgttacacgccgctcggccagagcttcccacccagggcacattctccaacgtgtccaccaatgtgtcgggaggcggcggatcgtcccagttcagagtgtcccctctggaccgcacctggaacctgggcgagaccgtggagctgaaatgtcaggtcctgctgagcaacccgacctccgggtgcagttggctgttccagccgcgtggtgctgccgcaagccctacgttcctgctttacctgagccagaacaagcccaaggcggccgagggcctggacacccagagattctccggcaagcgcctgggggacacattcgtgcttactttgagcgatttccgcagagagaacgagggctactatttctgttcggcgctgagcaattccatcatgtatttcagccactttgtgccagtgttcctgcctgccaagcctaccacaacaccagctccccgtcccccgactccggcgcctaccatcgcgagtcaaccgttgagcctgaggcctgaggcttgtcggcccgctgcggggggtgccgtccacaccaggggcctcgactttgcgtgcgacatctatatttgggcgcctctggcgggtacctgcggggtgctgctgctgtcattggtgattaccctgtactgcaatcaccgcaaccgccggcgggtctgtaagtgcccacggcctgtggtcaagtccggtgacaaaccgtcgctctcggctcgctacgtgcgcgctaagcgcagcggttccggggccaccaacttttcattgctgaagcaggccggtgatgtggaggagaatccagggcccatgcgccccaggctttggctccttcttgctgctcagctcactgtcttgcatggcaactccgttctgcagcagactcccgcctacatcaaggtgcagacgaacaagatggtgatgctgtcatgcgaggccaagatctctctttcaaatatgagaatttattggctacgacagcgccaggccccctccagcgacagccaccacgagttcctggcgctttgggattctgctaaaggcaccatccatggagaggaggtggaacaggagaagatagctgtcttccgcgacgcatcccgcttcatcctgaacctgaccagcgtgaagccggaggacagcggcatctacttctgtatgatcgttggctcccccgagctgaccttcggcaaaggcacccagctgtccgtggtggacttcctgcccaccacagcccagccaaccaagaaatccaccctcaagaagcgcgtgtgccgactgccccgccctgaaacccagaagggccctctgtgctcccccatcacccttggactgctggtggcgggagtcctggtgctgctcgtatctctgggtgtcgccatccacctgtgctgccgccgccgccgcgcccgcctgaggtttatgaaacagttttacaagtgataaatcgatggaagggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattactagtggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaactagtccagtgtggtggaattctgcagatatcacggctagcgccaccatgggtcgggggctgctcaggggcctgtggccgctgcacatcgtcctgtggacgcgtatcgccagcacgatcccaccgcacgttcagaagtcggtgaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggagaagaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgtgaaccggcagcagaaggctagtggttcaggcgcaacgaatttctctttgctgaagcaggctggggatgtcgaagaaaatccgggtccaatggtgggctcgctcaactgcatcgtagcagtctcccagaatatgggcatcgggaagaacggtgatttcccgtggcccccacttcgcaacgagagccgttatttccaaagaatgactacaacctcctccgtggagggtaagcagaacctggtcatcatggggaagaagacctggttctctatccctgaaaaaaaccgccccctgaagggccgcatcaacctggtgctgagcagggaactcaaggagcctcctcagggcgcgcattttctgagccgctcattggatgacgctctcaaactgaccgaacagccggagctagccaacaaggtggacatggtgtggatcgtcggaggctcctccgtgtacaaggaggccatgaatcaccccggccacttgaagctgttcgtcacccggatcatgcaggacttcgagtcggacacgttctttccagagattgacctggagaagtacaagctgctgcccgagtacccgggagttcttagtgatgtgcaggaggagaaaggcatcaagtacaaatttgaggtgtacgagaagaacgactaacggtccgtcctgaccaatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtatacaggttacctcagtctcctaggtacgtcttatatctatgaaaaaacattcaaaagcacaacatctagaagaacttaccttttttcaccactctattgcaaagatatgtaccgatttctctcgaagtacaaaaaaccgctagttttcaaattcacctcaagactttgaaaaaaaattgaatctgtcaatgtcaaataaaatcagaaacaaatgtcataatgttacgttaatgttgtcaggtcgaaaaataaaattgcaaatagaaattttgttccttttttattggtttttattggtgggaaaaatattccctctaactgcaaaagggttaattatgttagaggtagagtcgacaagctt

SEQ ID NO: 21 - DNA, 7332 bp, synthetic construct
  source 1..7332: mol_type = other DNA
gctagctggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacatactatcaacaggttgaactgctgatctgtacagtagaattggtaaagagagttgtgtaaaatattgagttcgcacatcttgttgtctgattattgatttttggcgaaaccatttgatcatatgacaagatgtgtatctaccttaacttaatgattttgataaaaatcattaggtaccaattacattgcttgcaattaaccctttaacggttataaggatctagatgagatagaaagatttggttttcggatttgtgttacataagatgcctaaaataaaaattgagattcaattttttttaaacttttttttaattggtggtaagaatattccctctacctgtttgagagtaatgaaattgtagtatgatttttcaacaaactaaaaaaacaacataaatctcacataataactttatttcaatcacacaattgaataccaataggttgacagtacttaccagcctgcaggtgaaagaccccacctgtaggtttggcaagttagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagcgaaccatcagatgtttccagggtgccccaaggacctgaaatgaccctgtgccttatttgaactaaccaatcagtttgcttcttgcttctgtttgtgtgcttctgctccctgagctcaataaaagagcccacaacccctcacttggtgggccagtcctctgatagactgtgtcccctggatacccgtacggtaccgctagcgccaccatgggacccaggctcctcttctgggcactgctttgtctcctcggaacaggcccagtggaggctggagtcacacaaagtcccacacacctgatcaaaacgagaggacagcaagcgactctgagatgctctcctatctctgggcacaccagtgtgtactggtaccaacaggccctgggtctgggcctccagttcctcctttggtatgacgagggtgaagagagaaacagaggaaacttccctcctagattttcaggtcgccagttccctaattatagctctgagctgaatgtgaacgccttggagctggaggactcggccctgtatctctgtgcttcctcacttgggcaattgaacacagaggcattctttggacaaggcaccagactcacagttgtagaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggtgcgacaaactttagcctgttgaaacaagccggcgacgttgaagagaaccccggacctatggaaaaaatgctcgagtgcgccttcatcgtgctttggctgcagctcggatggctgagcggagaggatcaagtgacacagtctcccgaggctctgaggctgcaagagggcgaaagcagctccctgaattgcagctacaccgtgtctggcctgaggggcctgttttggtacagacaagaccctggcaagggacccgagttcctgttcacactgtactctgccggcgaagaaaaagagaaagagcgcctgaaagcaaccctgaccaagaaagagagcttcctgcacatcacagcccctaagccagaggacagcgctacttacctgtgtgccgtttcatacggccagaatttcgtttttggtcccggaaccagattgtccgtgctgccctacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagcagcagggctaagaggtccggcagcggagccaccaatttttccctgctgaaacaggctggtgacgtggaagaaaaccctggccccatggcgctgcccgtcaccgcgctgctgctgcccctggcgctgctgttacacgccgctcggccagagcttcccacccagggcacattctccaacgtgtccaccaatgtgtcgggaggcggcggatcgtcccagttcagagtgtcccctctggaccgcacctggaacctgggcgagaccgtggagctgaaatgtcaggtcctgctgagcaacccgacctccgggtgcagttggctgttccagccgcgtggtgctgccgcaagccctacgttcctgctttacctgagccagaacaagcccaaggcggccgagggcctggacacccagagattctccggcaagcgcctgggggacacattcgtgcttactttgagcgatttccgcagagagaacgagggctactatttctgttcggcgctgagcaattccatcatgtatttcagccactttgtgccagtgttcctgcctgccaagcctaccacaacaccagctccccgtcccccgactccggcgcctaccatcgcgagtcaaccgttgagcctgaggcctgaggcttgtcggcccgctgcggggggtgccgtccacaccaggggcctcgactttgcgtgcgacatctatatttgggcgcctctggcgggtacctgcggggtgctgctgctgtcattggtgattaccctgtactgcaatcaccgcaaccgccggcgggtctgtaagtgcccacggcctgtggtcaagtccggtgacaaaccgtcgctctcggctcgctacgtgcgcgctaagcgcagcggttccggggccaccaacttttcattgctgaagcaggccggtgatgtggaggagaatccagggcccatgcgccccaggctttggctccttcttgctgctcagctcactgtcttgcatggcaactccgttctgcagcagactcccgcctacatcaaggtgcagacgaacaagatggtgatgctgtcatgcgaggccaagatctctctttcaaatatgagaatttattggctacgacagcgccaggccccctccagcgacagccaccacgagttcctggcgctttgggattctgctaaaggcaccatccatggagaggaggtggaacaggagaagatagctgtcttccgcgacgcatcccgcttcatcctgaacctgaccagcgtgaagccggaggacagcggcatctacttctgtatgatcgttggctcccccgagctgaccttcggcaaaggcacccagctgtccgtggtggacttcctgcccaccacagcccagccaaccaagaaatccaccctcaagaagcgcgtgtgccgactgccccgccctgaaacccagaagggccctctgtgctcccccatcacccttggactgctggtggcgggagtcctggtgctgctcgtatctctgggtgtcgccatccacctgtgctgccgccgccgccgcgcccgcctgaggtttatgaaacagttttacaagtgataaatcgatggaagggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattactagtggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatggtgggctcgctcaactgcatcgtagcagtctcccagaatatgggcatcgggaagaacggtgatttcccgtggcccccacttcgcaacgagagccgttatttccaaagaatgactacaacctcctccgtggagggtaagcagaacctggtcatcatggggaagaagacctggttctctatccctgaaaaaaaccgccccctgaagggccgcatcaacctggtgctgagcagggaactcaaggagcctcctcagggcgcgcattttctgagccgctcattggatgacgctctcaaactgaccgaacagccggagctagccaacaaggtggacatggtgtggatcgtcggaggctcctccgtgtacaaggaggccatgaatcaccccggccacttgaagctgttcgtcacccggatcatgcaggacttcgagtcggacacgttctttccagagattgacctggagaagtacaagctgctgcccgagtacccgggagttcttagtgatgtgcaggaggagaaaggcatcaagtacaaatttgaggtgtacgagaagaacgactaacggtccgtcctgaccaatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtatacaggttacctcagtctcctaggtacgtcttatatctatgaaaaaacattcaaaagcacaacatctagaagaacttaccttttttcaccactctattgcaaagatatgtaccgatttctctcgaagtacaaaaaaccgctagttttcaaattcacctcaagactttgaaaaaaaattgaatctgtcaatgtcaaataaaatcagaaacaaatgtcataatgttacgttaatgttgtcaggtcgaaaaataaaattgcaaatagaaattttgttccttttttattggtttttattggtgggaaaaatattccctctaactgcaaaagggttaattatgttagaggtagagtcgac

SEQ ID NO: 22 - DNA, 822 bp, synthetic construct
  source 1..822: mol_type = other DNA
atgaagaccttcgccggcttcagcttcctgttcctgtggctgcagctggactgcatgagcaggggcgaggacgtggaacagagcctgtttctgagcgtgcgcgagggcgacagcagcgtgatcaattgcacctacaccgacagctccagcacctacctgtactggtacaagcaggaacctggcgccggactgcagctgctgacctacatcttcagcaacatggacatgaagcaggaccagagactgaccgtgctgctgaacaagaaggacaagcacctgagcctgcggatcgccgatacccagacaggcgacagcgccatctacttttgcgccgagagcatcggcagcaacagcggctacgccctgaacttcggcaagggcacaagcctgctcgtgacccctcacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagc

SEQ ID NO: 23 - protein, 274 aa, synthetic construct
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAESIGSNSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 24 - DNA, 957 bp, synthetic construct
  source 1..957: mol_type = other DNA
atgggaatcagactgctgtgcagagtggccttctgcttcctggccgtgggcctggtggacgtgaaagtgacccagagcagcagatacctcgtgaagcggaccggcgagaaggtgttcctggaatgcgtgcaggacatggaccacgagaatatgttctggtacagacaggaccccggcctgggcctgcggctgatctacttcagctacgacgtgaagatgaaggaaaagggcgacatccccgagggctacagcgtgtccagagagaagaaagagcggttcagcctgatcctggaaagcgccagcaccaaccagaccagcatgtacctgtgcgccagcagaggcctggccggctacgagcagtattttggccctggcacccggctgaccgtgaccgaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggt

SEQ ID NO: 25 - protein, 319 aa, synthetic construct
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASRGLAGYEQYFGPGTRLTVTEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSGRAKRSGSG

SEQ ID NO: 26 - DNA, 1836 bp, synthetic construct
  source 1..1836: mol_type = other DNA
atgggaatcagactgctgtgcagagtggccttctgcttcctggccgtgggcctggtggacgtgaaagtgacccagagcagcagatacctcgtgaagcggaccggcgagaaggtgttcctggaatgcgtgcaggacatggaccacgagaatatgttctggtacagacaggaccccggcctgggcctgcggctgatctacttcagctacgacgtgaagatgaaggaaaagggcgacatccccgagggctacagcgtgtccagagagaagaaagagcggttcagcctgatcctggaaagcgccagcaccaaccagaccagcatgtacctgtgcgccagcagaggcctggccggctacgagcagtattttggccctggcacccggctgaccgtgaccgaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggtgcgacaaactttagcctgttgaaacaagccggcgacgttgaagagaaccccggacctatgaagaccttcgccggcttcagcttcctgttcctgtggctgcagctggactgcatgagcaggggcgaggacgtggaacagagcctgtttctgagcgtgcgcgagggcgacagcagcgtgatcaattgcacctacaccgacagctccagcacctacctgtactggtacaagcaggaacctggcgccggactgcagctgctgacctacatcttcagcaacatggacatgaagcaggaccagagactgaccgtgctgctgaacaagaaggacaagcacctgagcctgcggatcgccgatacccagacaggcgacagcgccatctacttttgcgccgagagcatcggcagcaacagcggctacgccctgaacttcggcaagggcacaagcctgctcgtgacccctcacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagc

SEQ ID NO: 27 - protein, 612 aa, synthetic construct
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASRGLAGYEQYFGPGTRLTVTEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSGRAKRSGSGATNFSLLKQAGDVEENPGPMKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAESIGSNSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 28 - DNA, 795 bp, synthetic construct
  source 1..795: mol_type = other DNA
atgaagctggtgaccagcatcaccgtgctgctgagcctgggcatcatgggcgacgccaagaccacccagcccaacagcatggagagcaacgaggaggagcccgtgcacctgccctgcaaccacagcaccatcagcggcaccgactacatccactggtacaggcagctgcccagccagggccccgagtacgtgatccacggcctgaccagcaacgtgaacaacaggatggccagcctggccatcgccgaggacaggaagagcagcaccctgatcctgcacagggccaccctgagggacgccgccgtgtactactgcatcctgttcaacttcaacaagttctacttcggcagcggcaccaagctgaacgtgaagcccaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagc

SEQ ID NO: 29 - protein, 265 aa, synthetic construct
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILFNFNKFYFGSGTKLNVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 30 - DNA, 960 bp, synthetic construct
  source 1..960: mol_type = other DNA
atgggccccggcctgctgcactggatggccctgtgcctgctgggcaccggccacggcgacgccatggtgatccagaaccccaggtaccaggtgacccagttcggcaagcccgtgaccctgagctgcagccagaccctgaaccacaacgtgatgtactggtaccagcagaagagcagccaggcccccaagctgctgttccactactacgacaaggacttcaacaacgaggccgacacccccgacaacttccagagcaggaggcccaacaccagcttctgcttcctggacatcaggagccccggcctgggcgacgccgccatgtacctgtgcgccaccagcagcggcgagaccaacgagaagctgttcttcggcagcggcacccagctgagcgtgctggaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggt

SEQ ID NO: 31 - protein, 320 aa, synthetic construct
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSSGETNEKLFFGSGTQLSVLEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSGRAKRSGSG

SEQ ID NO: 32 - DNA, 1812 bp, synthetic construct
  source 1..1812: mol_type = other DNA
atgggccccggcctgctgcactggatggccctgtgcctgctgggcaccggccacggcgacgccatggtgatccagaaccccaggtaccaggtgacccagttcggcaagcccgtgaccctgagctgcagccagaccctgaaccacaacgtgatgtactggtaccagcagaagagcagccaggcccccaagctgctgttccactactacgacaaggacttcaacaacgaggccgacacccccgacaacttccagagcaggaggcccaacaccagcttctgcttcctggacatcaggagccccggcctgggcgacgccgccatgtacctgtgcgccaccagcagcggcgagaccaacgagaagctgttcttcggcagcggcacccagctgagcgtgctggaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggtgcgacaaactttagcctgttgaaacaagccggcgacgttgaagagaaccccggacctatgaagctggtgaccagcatcaccgtgctgctgagcctgggcatcatgggcgacgccaagaccacccagcccaacagcatggagagcaacgaggaggagcccgtgcacctgccctgcaaccacagcaccatcagcggcaccgactacatccactggtacaggcagctgcccagccagggccccgagtacgtgatccacggcctgaccagcaacgtgaacaacaggatggccagcctggccatcgccgaggacaggaagagcagcaccctgatcctgcacagggccaccctgagggacgccgccgtgtactactgcatcctgttcaacttcaacaagttctacttcggcagcggcaccaagctgaacgtgaagcccaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagc

SEQ ID NO: 33 - protein, 604 aa, synthetic construct
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSSGETNEKLFFGSGTQLSVLEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSGRAKRSGSGATNFSLLKQAGDVEENPGPMKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILFNFNKFYFGSGTKLNVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 34 - protein, 20 aa, synthetic construct
  VARIANT 1..10: note = This region may encompass 0-10 residues
  VARIANT 11..20: note = This region may encompass 0-10 residues
GGGGGGGGGGSSSSSSSSSS

SEQ ID NO: 35 - protein, 10 aa, synthetic construct
GGGGSGGGGS

SEQ ID NO: 36 - protein, 8 aa, synthetic construct
GGGSGGGS

SEQ ID NO: 37 - protein, 11 aa, synthetic construct
GGGSGGGSGGS

SEQ ID NO: 38 - protein, 9 aa, unidentified
LAGLIDADG

SEQ ID NO: 39 - DNA, 20 bp, synthetic construct
  source 1..20: mol_type = other DNA
cggctactacaaccagagcg

SEQ ID NO: 40 - DNA, 20 bp, synthetic construct
  source 1..20: mol_type = other DNA
agatcacactgacctggcag

SEQ ID NO: 41 - DNA, 20 bp, synthetic construct
  source 1..20: mol_type = other DNA
aggtcagtgtgatctccgca

SEQ ID NO: 42 - DNA, 20 bp, synthetic construct
  source 1..20: mol_type = other DNA
ggccacggagcgagacatct

SEQ ID NO: 43 - protein, 8 aa, Homo sapiens
VRFFFPSL

(19) United States
(12) Patent Application Publication
(10) Pub. No.: US 2026/0232804 A1
(43) Pub. Date: Aug. 13, 2026

(54) MAGEA1 IMMUNOGENIC PEPTIDES, BINDING PROTEINS RECOGNIZING MAGEA1 IMMUNOGENIC PEPTIDES, AND USES THEREOF

(71) Applicant: TScan Therapeutics, Inc., Waltham, MA (US)

(72) Inventors: Nancy Nabilsi, Acton, MA (US); Yifan Wang, Lexington, MA (US); Jenny Tadros, Brighton, MA (US); Mollie M. Jurewicz, Auburn, MA (US); Gavin MacBeath, Wakefield, MA (US)

(21) Appl. No.: 19/118,633

(22) PCT Filed: Oct. 5, 2023
(86) PCT No.: PCT/US2023/076072

§ 371 Date: Apr. 4, 2025

Related U.S. Application Data
(60) Provisional application No. 63/413,560, filed on Oct. 5, 2022.
(60) Provisional application No. 63/468,842, filed on May 25, 2023.

(51) Int. Cl. A61K 40/42; A61K 38/00; A61K 40/11; A61K 40/32; A61P 35/00; C07K 14/705; C07K 16/30; G01N 33/575

(52) U.S. Cl. CPC A61K 40/4268; A61K 40/11; A61K 40/32; A61P 35/00; C07K 14/7051; C07K 16/30; G01N 33/5758; A61K 38/00; C07K 2317/21; C07K 2317/24

(57) ABSTRACT

Provided herein are MAGEA1 immunogenic peptides, binding proteins recognizing MAGEA1 immunogenic peptides, and uses thereof.

### CROSS-REFERENCE TO RELATED APPLICATIONS

This application is the U.S. national phase of International Patent Application No. PCT/US23/76072, filed on 5 Oct. 2023, which claims the benefit of priority to U.S. Provisional Application Ser. No. 63/413,560, filed on 5 Oct. 2022, and U.S. Provisional Application Ser. No. 63/468,842, filed on 25 May 2023; the entire contents of each of said applications are incorporated herein in its entirety by this reference.

### SEQUENCE LISTING

The application contains a Sequence Listing which has been submitted electronically in .XML format. Said .XML copy, created on Oct. 17, 2023, is named “TTC-01325_SL.xml” and 92,133 bytes in size. The sequence listing contained in this .XML file is part of the specification and is hereby incorporated by reference herein in its entirety.

### BACKGROUND OF THE INVENTION

Adoptive cell transfer (ACT) using engineered T cells has demonstrated great efficacy in treating certain types of liquid tumor and holds promise for treating solid tumor. T cell receptor-engineered T cells (TCR-T) are T cells expressing an exogenous TCR that recognizes an antigen that exist in cancer cells. The TCR-antigen interaction is the core component of the targeting mechanism that allows the TCR-T cells to kill cancer cells. One of the challenges for the broad testing and adoption of TCR-T therapy is the lack of TCR-antigen pairs that are applicable to a wide range of patients and indications.

In addition, the number of pursued antigens is limited due to the difficulty of discovering novel TCR-antigen pairs which commonly require prediction of the MHC presented epitope. However, such epitopes may not be immunogenic, thereby making it difficult to identify a reactive TCR, or the epitope may not be processed and presented physiologically by the cancer cells. Accordingly, there is a great need in the art to identify TCR-antigen pairs in the context of a variety of widely applicable HLA alleles in order to develop useful reagents to diagnose, prognose, treat, and screen agents relevant for disorders characterized by the expression of the antigens.

### SUMMARY OF THE INVENTION

The present invention is based, at least in part, on the discovery of MAGEA1 immunogenic peptides and binding proteins recognizing such MAGEA1 immunogenic peptides based on unbiased functional screens used to discover the antigen of TCR clonotypes identified from subjects having disorders associated with MAGEA1 expression (e.g., subjects afflicted with a melanoma, head & neck cancer, lung cancer, cervical cancer, hepatocellular carcinoma, colorectal cancer, gastrointestinal cancer, breast invasive carcinoma, or bladder urothelial carcinoma). The identified TCRs recognized MAGEA1 immunogenic peptides, such as those listed in Table 1, in the context of a variety of HLA alleles (e.g., HLA-A*02:01). MAGEA1 is demonstrated herein to be selectively expressed in cancer and testis tissue, but not in normal somatic tissues, thereby making it an ideal target for ACT. The ability of MAGEA1 binding proteins (e.g., TCRs described herein) to bind MAGEA1 immunogenic peptides and to elicit immune responses that kill cells expressing MAGEA1 (e.g., cancer cells) demonstrates the utility of such binding proteins in a diversity of uses, including methods of diagnosis, prognosis, treatment, and screening of agents relevant for disorders characterized by MAGEA1 expression.

In one aspect, an immunogenic peptide comprising a peptide epitope selected from peptide sequences listed in Table 1, is provided.

In another aspect, an immunogenic peptide consisting of a peptide epitope selected from peptide sequences listed in Table 1, is provided.

Numerous embodiments are further provided that may be applied to any aspect encompassed by the present invention and/or combined with any other embodiment described herein. For example, in one embodiment, the immunogenic peptide is derived from a MAGEA1 protein, optionally wherein the immunogenic peptide is 8, 9, 10, 11, 12, 13, 14, 15, 16, 17, 18, 19, 20, 21, 22, 23, 24, or 25 amino acids in length. In another embodiment, the immunogenic peptide is capable of eliciting an immune response against MAGEA1 and/or MAGEA1-expressing cells in a subject, optionally wherein the immune response is i) a T cell response and/or a CD8+ T cell response and/or ii) selected from the group consisting of T cell expansion (e.g., proliferation), cytokine release, and/or cytotoxic killing.

In still another aspect, an immunogenic composition comprising at least one immunogenic peptide described herein, is provided.

Numerous embodiments are further provided that may be applied to any aspect encompassed by the present invention and/or combined with any other embodiment described herein. For example, in one embodiment, the immunogenic composition further comprises an adjuvant. In another embodiment, the immunogenic composition is capable of eliciting an immune response against MAGEA1 and/or MAGEA1-expressing cells in a subject, optionally wherein the immune response is i) a T cell response and/or a CD8+ T cell response and/or ii) selected from the group consisting of T cell expansion (e.g., proliferation), cytokine release, and/or cytotoxic killing.

In yet another aspect, a composition comprising a peptide epitope selected from peptide sequences listed in Table 1, and an MHC molecule, is provided.

Numerous embodiments are further provided that may be applied to any aspect encompassed by the present invention and/or combined with any other embodiment described herein. For example, in one embodiment, the MHC molecule is an MHC multimer, optionally wherein the MHC multimer is a tetramer. In another embodiment, the MHC molecule is an MHC class I molecule. In still another embodiment, the MHC molecule comprises an MHC alpha chain that is an HLA serotype selected from the group consisting of HLA-A*02, HLA-A*03, HLA-A*01, HLA-A*11, HLA-A*24, HLA-B*07, HLA-C*07, HLA-C*01, HLA-C*02, HLA-C*03, HLA-C*04, HLA-C*05, HLA-C*06, HLA-C*08, HLA-C*12, HLA-C*14, HLA-C*15, HLA-C*16, HLA-C*17, and HLA-C*18, optionally wherein the HLA allele is selected from the group consisting of HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:12, HLA-A*02:13, HLA-A*02:14, HLA-A*02:16, HLA-A*02:17, HLA-A*02:19, HLA-A*02:20, HLA-A*02:22, HLA-A*02:24, HLA-A*02:30, HLA-A*02:42, HLA-A*02:53, HLA-A*02:60, HLA-A*02:74 allele, HLA-A*03:01, HLA-A*03:02, HLA-A*03:05, HLA-A*03:07, HLA-A*01:01, HLA-A*01:02, HLA-A*01:03, HLA-A*01:16 allele, HLA-A*11:01, HLA-A*11:02, HLA-A*11:03, HLA-A*11:04, HLA-A*11:05, HLA-A*11:19 allele, HLA-A*24:02, HLA-A*24:03, HLA-A*24:05, HLA-A*24:07, HLA-A*24:08, HLA-A*24:10, HLA-A*24:14, HLA-A*24:17, HLA-A*24:20, HLA-A*24:22, HLA-A*24:25, HLA-A*24:26, HLA-A*24:58 allele, HLA-B*07:02, HLA-B*07:04, HLA-B*07:05, HLA-B*07:09, HLA-B*07:10, HLA-B*07:15, HLA-B*07:21, HLA-C*07:02, HLA-C*07:01, HLA-C*04:01, HLA-C*06:02, HLA-C*03:04, HLA-C*05:01, HLA-C*16:01, HLA-C*02:02, HLA-C*03:03, HLA-C*12:03, HLA-C*08:02, HLA-C*01:02, HLA-C*17:01, HLA-C*15:02, HLA-C*14:02, HLA-C*12:02, HLA-C*07:04, HLA-C*08:01, HLA-C*03:02, HLA-C*18:01, HLA-C*15:05, HLA-C*16:02, HLA-C*08:04, HLA-C*03:05, and HLA-C*14:03 allele. In yet another embodiment, the HLA serotype is HLA-A*02, such as HLA-A*02:01.

In another aspect, a stable MHC-peptide complex, comprising an immunogenic peptide described herein in the context of an MHC molecule, is provided.

Numerous embodiments are further provided that may be applied to any aspect encompassed by the present invention and/or combined with any other embodiment described herein. For example, in one embodiment, the MHC molecule is an MHC multimer, optionally wherein the MHC multimer is a tetramer. In another embodiment, the MHC molecule is an MHC class I molecule. In still another embodiment, the MHC molecule comprises an MHC alpha chain that is an HLA serotype selected from the group consisting of HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:12, HLA-A*02:13, HLA-A*02:14, HLA-A*02:16, HLA-A*02:17, HLA-A*02:19, HLA-A*02:20, HLA-A*02:22, HLA-A*02:24, HLA-A*02:30, HLA-A*02:42, HLA-A*02:53, HLA-A*02:60, HLA-A*02:74 allele, HLA-A*03:01, HLA-A*03:02, HLA-A*03:05, HLA-A*03:07, HLA-A*01:01, HLA-A*01:02, HLA-A*01:03, HLA-A*01:16 allele, HLA-A*11:01, HLA-A*11:02, HLA-A*11:03, HLA-A*11:04, HLA-A*11:05, HLA-A*11:19 allele, HLA-A*24:02, HLA-A*24:03, HLA-A*24:05, HLA-A*24:07, HLA-A*24:08, HLA-A*24:10, HLA-A*24:14, HLA-A*24:17, HLA-A*24:20, HLA-A*24:22, HLA-A*24:25, HLA-A*24:26, HLA-A*24:58 allele, HLA-B*07:02, HLA-B*07:04, HLA-B*07:05, HLA-B*07:09, HLA-B*07:10, HLA-B*07:15, HLA-B*07:21, HLA-C*07:02, HLA-C*07:01, HLA-C*04:01, HLA-C*06:02, HLA-C*03:04, HLA-C*05:01, HLA-C*16:01, HLA-C*02:02, HLA-C*03:03, HLA-C*12:03, HLA-C*08:02, HLA-C*01:02, HLA-C*17:01, HLA-C*15:02, HLA-C*14:02, HLA-C*12:02, HLA-C*07:04, HLA-C*08:01, HLA-C*03:02, HLA-C*18:01, HLA-C*15:05, HLA-C*16:02, HLA-C*08:04, HLA-C*03:05, and HLA-C*14:03 allele. In yet another embodiment, the peptide epitope and the MHC molecule are covalently linked and/or wherein the alpha and beta chains of the MHC molecule are covalently linked. In another embodiment, the stable MHC-peptide complex comprises a detectable label, optionally wherein the detectable label is a fluorophore.

In still another aspect, an immunogenic composition comprising a stable MHC-peptide complex described herein, and an adjuvant, is provided.

In yet another aspect, an isolated nucleic acid that encodes an immunogenic peptide described herein, or a complement thereof, is provided.

In another aspect, a vector comprising an isolated nucleic acid described herein, is provided.

In still another aspect, a cell that a) comprises an isolated nucleic acid described herein, b) comprises a vector described herein, and/or c) produces one or more immunogenic peptides described herein and/or presents at the cell surface one or more stable MHC-peptide complexes described herein, optionally wherein the cell is genetically engineered, is provided.

In yet another aspect, a device or kit comprising a) one or more immunogenic peptides described herein and/or b) one or more stable MHC-peptide complexes described herein, said device or kit optionally comprising a reagent to detect binding of a) and/or b) to a binding protein, optionally wherein the binding protein is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain, is provided.

In another aspect, a method of detecting T cells that bind a stable MHC-peptide complex comprising: a) contacting a sample comprising T cells with a stable MHC-peptide complex described herein; and b) detecting binding of T cells to the stable MHC-peptide complex, optionally further determining the percentage of stable MHC-peptide-specific T cells that bind to the stable MHC-peptide complex, optionally wherein the sample comprises peripheral blood mononuclear cells (PBMCs), is provided.

Numerous embodiments are further provided that may be applied to any aspect encompassed by the present invention and/or combined with any other embodiment described herein. For example, in one embodiment, T cells are CD8+ T cells. In another embodiment, detecting and/or determining is performed using fluorescence activated cell sorting (FACS), enzyme linked immunosorbent assay (ELISA), radioimmune assay (RIA), immunochemically, Western blot, or intracellular flow assay. In still another embodiment, a sample comprises T cells contacted with, or suspected of having been contacted with, one or more MAGEA1 proteins or fragments thereof.

In still another aspect, a method of determining whether a T cell has had exposure to MAGEA1 comprising: a) incubating a cell population comprising T cells with an immunogenic peptide described herein or a stable MHC-peptide complex described herein; and b) detecting the presence or level of reactivity, wherein the presence of or a higher level of reactivity compared to a control level indicates that the T cell has had exposure to MAGEA1, optionally wherein the cell population comprising T cells is obtained from a subject, is provided.

In yet another aspect, a method for predicting the clinical outcome of a subject afflicted with a disorder characterized by MAGEA1 expression comprising: a) determining the presence or level of reactivity between T cells obtained from the subject and one more immunogenic peptides described herein or one or more stable MHC-peptide complexes described herein; and b) comparing the presence or level of reactivity to that from a control, wherein the control is obtained from a subject having a good clinical outcome, wherein the presence or a higher level of reactivity in the subject sample as compared to the control indicates that the subject has a good clinical outcome, is provided.

In another aspect, a method of assessing the efficacy of a therapy for a disorder characterized by MAGEA1 expression comprising: a) determining the presence or level of reactivity between T cells obtained from the subject and one more immunogenic peptides described herein or one or more stable MHC-peptide complexes described herein, in a first sample obtained from the subject prior to providing at least a portion of the therapy to the subject, and b) determining the presence or level of reactivity between the one more immunogenic peptides described herein, or the one or more stable MHC-peptide complexes described herein, and T cells obtained from the subject present in a second sample obtained from the subject following provision of the therapy to the subject, wherein the presence or a higher level of reactivity in the second sample, relative to the first sample, is an indication that the therapy is efficacious for treating the disorder characterized by MAGEA1 expression in the subject, is provided.

Numerous embodiments are further provided that may be applied to any aspect encompassed by the present invention and/or combined with any other embodiment described herein. For example, in one embodiment, the level of reactivity is indicated by a) the presence of binding and/or b) T cell activation and/or effector function, optionally wherein the T cell activation or effector function is T cell proliferation, killing, or cytokine release. In another embodiment, a method further comprises repeating steps a) and b) at a subsequent point in time, optionally wherein the subject has undergone treatment to ameliorate the disorder characterized by MAGEA1 expression between the first point in time and the subsequent point in time. In still another embodiment, T cell binding, activation, and/or effector function is detected using fluorescence activated cell sorting (FACS), enzyme linked immunosorbent assay (ELISA), radioimmune assay (RIA), immunochemically, Western blot, or intracellular flow assay. In yet another embodiment, a control level is a reference number. In another embodiment, a control level is a level of a subject without the disorder characterized by MAGEA1 expression.

In still another aspect, a method of preventing and/or treating a disorder characterized by MAGEA1 expression in a subject comprising administering to the subject a therapeutically effective amount of a composition described herein.

In yet another aspect, a method of identifying a peptide-binding molecule, or antigen-binding fragment thereof, that binds to a peptide epitope selected from the peptide sequences listed in Table 1 comprising: a) providing a cell presenting a peptide epitope selected from the peptide sequences listed in Table 1 in the context of an MHC molecule on the surface of the cell; b) determining binding of a plurality of candidate peptide-binding molecules or antigen-binding fragments thereof to the peptide epitope in the context of the MHC molecule on the cell; and c) identifying one or more peptide-binding molecules or antigen-binding fragments thereof that bind to the peptide epitope in the context of the MHC molecule, is provided.

Numerous embodiments are further provided that may be applied to any aspect encompassed by the present invention and/or combined with any other embodiment described herein. For example, in one embodiment, a step a) comprises contacting the MHC molecule on the surface of the cell with a peptide epitope selected from the peptide sequences listed in Table 1. In another embodiment, a step a) comprises expressing the peptide epitope selected from the peptide sequences listed in Table 1 in the cell using a vector comprising a heterologous sequence encoding the peptide epitope.

In another aspect, a method of identifying a peptide-binding molecule or antigen-binding fragment thereof that binds to a peptide epitope selected from the peptide sequences listed in Table 1 comprising: a) providing a peptide epitope either alone or in a stable MHC-peptide complex, comprising a peptide epitope selected from the peptide sequences listed in Table 1, either alone or in the context of an MHC molecule; b) determining binding of a plurality of candidate peptide-binding molecules or antigen-binding fragments thereof to the peptide or stable MHC-peptide complex; and c) identifying one or more peptide-binding molecules or antigen-binding fragments thereof that bind to the peptide epitope or the stable MHC-peptide complex, optionally wherein the MHC or MHC-peptide complex is as described herein, is provided.

Numerous embodiments are further provided that may be applied to any aspect encompassed by the present invention and/or combined with any other embodiment described herein. For example, in one embodiment, a plurality of candidate peptide binding molecules comprises an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain. In another embodiment, a plurality of candidate peptide binding molecules comprises at least 2, 5, 10, 100, 10³, 10⁴, 10⁵, 10⁶, 10⁷, 10⁸, 10⁹, or more, different candidate peptide binding molecules. In still another embodiment, a plurality of candidate peptide binding molecules comprises one or more candidate peptide binding molecules that are obtained from a sample from a subject or a population of subjects; or the plurality of candidate peptide binding molecules comprises one or more candidate peptide binding molecules that comprise mutations in a parent scaffold peptide binding molecule obtained from a sample from a subject. In yet another embodiment, a subject or population of subjects are a) not afflicted with a disorder characterized by MAGEA1 expression and/or have recovered from a disorder characterized by MAGEA1 expression, or b) are afflicted with a disorder characterized by MAGEA1 expression. In another embodiment, a subject or population of subjects has been administered a composition described herein. In still another embodiment, a subject is an animal model of a disorder characterized by MAGEA1 expression and/or a mammal, optionally wherein the mammal is a human, a primate, or a rodent. In yet another embodiment, a subject is an animal model of a disorder characterized by MAGEA1 expression, an HLA-transgenic mouse, and/or a human TCR transgenic mouse. In another embodiment, a sample comprises peripheral blood mononuclear cells (PBMCs), T cells, and/or CD8+ memory T cells.

In still another aspect, a peptide-binding molecule or antigen-binding fragment thereof identified according to a method described herein, optionally wherein the peptide-binding molecule or antigen-binding fragment thereof is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain, is provided.

In yet another aspect, a method of treating a disorder characterized by MAGEA1 expression in a subject comprising administering to the subject a therapeutically effective amount of genetically engineered T cells that express a peptide-binding molecule or antigen-binding fragment thereof that i) binds to a peptide epitope selected from the sequences listed in Table 1, ii) is identified according to a method described herein, and/or iii) binds to a stable MHC-peptide complex comprising a peptide epitopes selected from the sequences listed in Table 1 in the context of an MHC molecule, optionally wherein the peptide-binding molecule or antigen-binding fragment thereof is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain, optionally wherein the MHC or MHC-peptide complex is as described herein, is provided.

Numerous embodiments are further provided that may be applied to any aspect encompassed by the present invention and/or combined with any other embodiment described herein. For example, in one embodiment, T cells are isolated from a) the subject, b) a donor not afflicted with the disorder characterized by MAGEA1 expression, or c) a donor recovered from a disorder characterized by MAGEA1 expression.

In another aspect, a method of treating a disorder characterized by MAGEA1 expression in a subject comprising transfusing antigen-specific T cells to the subject, wherein the antigen-specific T cells are generated by: a) stimulating immune cells from a subject with a composition described herein; and b) expanding antigen-specific T cells in vitro or ex vivo, optionally i) isolating immune cells from the subject before stimulating the immune cells and/or ii) wherein the immune cells comprise PBMCs, T cells, CD8+ T cells, naive T cells, central memory T cells, and/or effector memory T cells, is provided.

Numerous embodiments are further provided that may be applied to any aspect encompassed by the present invention and/or combined with any other embodiment described herein. For example, in one embodiment, agents are placed in contact under conditions and for a time suitable for the formation of at least one immune complex between the peptide epitope, immunogenic peptide, stable MHC-peptide complex, T cell receptor, and/or immune cells. In another embodiment, a peptide epitope, immunogenic peptide, stable MHC-peptide complex, and/or T cell receptor is expressed by cells and the cells are expanded and/or isolated during one or more steps. In still another embodiment, a disorder characterized by MAGEA1 expression is a cancer or relapse thereof, optionally wherein the cancer is selected from the group consisting of melanoma, head & neck cancer, lung cancer, cervical cancer, hepatocellular carcinoma, colorectal cancer, gastrointestinal cancer, breast invasive carcinoma, and bladder urothelial carcinoma. In yet another embodiment, a subject is an animal model of a disorder characterized by MAGEA1 expression and/or a mammal, optionally wherein the mammal is a human, a primate, or a rodent.

In still another aspect, a binding protein that binds a polypeptide comprising an immunogenic peptide sequence described herein, an immunogenic peptide described herein, and/or the stable MHC-peptide complex described herein, optionally wherein the binding protein is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain, is provided.

Numerous embodiments are further provided that may be applied to any aspect encompassed by the present invention and/or combined with any other embodiment described herein. For example, in one embodiment, a binding protein comprises: a) a T cell receptor (TCR) alpha chain CDR sequence with at least about 80% identity to a TCR alpha chain CDR sequence selected from the group consisting of TCR alpha chain CDR sequences listed in Table 2; and/or b) a TCR beta chain CDR sequence with at least about 80% identity to a TCR beta chain CDR sequence selected from the group consisting of TCR beta chain CDR sequences listed in Table 2, wherein the binding protein is capable of binding to a MAGEA1 immunogenic peptide-MHC (pMHC) complex, optionally wherein the binding affinity has a K_{d} less than or equal to about 5×10⁻⁴ M. In another embodiment, a binding protein comprises: a) a TCR alpha chain variable (V_{α}) domain sequence with at least about 80% identity to a TCR V_{α} domain sequence selected from the group consisting of TCR V_{α} domain sequences listed in Table 2; and/or b) a TCR beta chain variable (V_{β}) domain sequence with at least about 80% identity to a TCR V_{β} domain sequence selected from the group consisting of TCR V_{β} domain sequences listed in Table 2, wherein the binding protein is capable of binding to a MAGEA1 immunogenic peptide-MHC (pMHC) complex, optionally wherein the binding affinity has a K_{d} less than or equal to about 5×10⁻⁴ M. In still another embodiment, a binding protein comprises: a) a TCR alpha chain sequence with at least about 80% identity to a TCR alpha chain sequence selected from the group consisting of TCR alpha chain sequences listed in Table 2; and/or b) a TCR beta chain sequence with at least about 80% identity to a TCR beta chain sequence selected from the group consisting of TCR beta chain sequences listed in Table 2, wherein the binding protein is capable of binding to a MAGEA1 immunogenic peptide-MHC (pMHC) complex, optionally wherein the binding affinity has a K_{d} less than or equal to about 5×10⁻⁴ M. In yet another embodiment, a binding protein comprises: a) a TCR alpha chain CDR sequence selected from the group consisting of TCR alpha chain CDR sequences listed in Table 2; and/or b) a TCR beta chain CDR sequence selected from the group consisting of TCR beta chain CDR sequences listed in Table 2, wherein the binding protein is capable of binding to a MAGEA1 immunogenic peptide-MHC (pMHC) complex, optionally wherein the binding affinity has a K_{d} less than or equal to about 5×10⁻⁴ M. In yet another embodiment, a binding protein comprises: a) a TCR alpha chain variable (V_{α}) domain sequence selected from the group consisting of TCR V_{α} domain sequences listed in Table 2; and/or b) a TCR beta chain variable (V_{β}) domain sequence selected from the group consisting of TCR V_{β} domain sequences listed in Table 2, wherein the binding protein is capable of binding to a MAGEA1 immunogenic peptide-MHC (pMHC) complex, optionally wherein the binding affinity has a K_{d} less than or equal to about 5×10⁻⁴ M, is provided. In another embodiment, a binding protein comprises: a) a TCR alpha chain sequence selected from the group consisting of TCR alpha chain sequences listed in Table 2; and/or b) a TCR beta chain sequence selected from the group consisting of TCR beta chain sequences listed in Table 2, wherein the binding protein is capable of binding to a MAGEA1 immunogenic peptide-MHC (pMHC) complex, optionally wherein the binding affinity has a K_{d} less than or equal to about 5×10⁻⁴ M, is provided. In another embodiment, 1) a TCR alpha chain CDR, TCR V_{α} domain, and/or TCR alpha chain is encoded by a TRAV, TRAJ, and/or TRAC gene or fragment thereof selected from the group of TRAV, TRAJ, and TRAC genes listed in Table 2, and/or 2) a TCR beta chain CDR, TCR V_{β} domain, and/or TCR beta chain is encoded by a TRBV, TRBJ, and/or TRBC gene or fragment thereof selected from the group of TRBV, TRBJ, and TRBC genes listed in Table 2, and/or 3) each CDR of the binding protein has up to five amino acid substitutions, insertions, deletions, or a combination thereof as compared to the cognate reference CDR sequence listed in Table 2. In still another embodiment, a binding protein is chimeric, humanized, or human. In yet another embodiment, a binding protein comprises a binding domain having a transmembrane domain, and an effector domain that is intracellular. In another embodiment, a TCR alpha chain and a TCR beta chain are covalently linked, optionally wherein the TCR alpha chain and the TCR beta chain are covalently linked through a linker peptide. In still another embodiment, a TCR alpha chain and/or a TCR beta chain are covalently linked to a moiety, optionally wherein the covalently linked moiety comprises an affinity tag or a label. In yet another embodiment, an affinity tag is selected from the group consisting of aCD34 enrichment tag, glutathione-S-transferase (GST), calmodulin binding protein (CBP), protein C tag, Myc tag, HaloTag, HA tag, Flag tag, His tag, biotin tag, and V5 tag, and/or wherein the label is a fluorescent protein. In another embodiment, a covalently linked moiety is selected from the group consisting of an inflammatory agent, cytokine, toxin, cytotoxic molecule, radioactive isotope, or antibody or antigen-binding fragment thereof. In still another embodiment, a binding protein binds to the pMHC complex on a cell surface. In yet another embodiment, an MHC or MHC-peptide complex is as described herein. In another embodiment, binding of a binding protein to the MAGEA1 peptide-MHC (pMHC) complex elicits an immune response, optionally wherein the immune response is i) a T cell response and/or a CD8+ T cell response and/or ii) selected from the group consisting of T cell expansion, cytokine release, and/or cytotoxic killing. In still another embodiment, a binding protein is capable of specifically and/or selectively binding to a MAGEA1 immunogenic peptide-MHC (pMHC) complex with a K_{d} less than or equal to about 1×10⁻⁴ M, less than or equal to about 5×10⁻⁵ M, less than or equal to about 1×10⁻⁵ M, less than or equal to about 5×10⁻⁶ M, less than or equal to about 1×10⁻⁶ M, less than or equal to about 5×10⁻⁷ M, less than or equal to about 1×10⁻⁷ M, less than or equal to about 5×10⁻⁸ M, less than or equal to about 1×10⁻⁸ M, less than or equal to about 5×10⁻⁹ M, less than or equal to about 1×10⁻⁹ M, less than or equal to about 5×10⁻¹⁰ M, less than or equal to about 1×10⁻¹⁰ M, less than or equal to about 5×10⁻¹¹ M, less than or equal to about 1×10⁻¹¹ M, less than or equal to about 5×10⁻¹² M, or less than or equal to about 1×10⁻¹² M. In yet another embodiment, a binding protein has a higher binding affinity to the peptide-MHC (pMHC) than does a known T-cell receptor, optionally wherein the higher binding affinity is at least 1.05-fold higher. In another embodiment, a binding protein induces higher T cell expansion, cytokine release, and/or cytotoxic killing than does a known T-cell receptor when contacted with target cells with a heterozygous expression of MAGEA1, optionally wherein the induction is at least 1.05-fold higher. As used herein, references to fold changes, in some embodiments, may be in comparison to any reference modality of interest, such as comparison to a different binding protein; comparison to the same binding protein under different context like expression of the same binding protein in a different immune cell, at a different level, in combination with other agents described herein; and the like. In still another embodiment, cytotoxic killing is of a target cancer cell. In yet another embodiment, cancer is selected from the group consisting of melanoma, head & neck cancer, lung cancer, cervical cancer, hepatocellular carcinoma, colorectal cancer, gastrointestinal cancer, breast invasive carcinoma, and bladder urothelial carcinoma. In another embodiment, a binding protein does not bind to a peptide-MHC (pMHC) complex comprising a PIEZO1, NBEAL1, NBEAL2, and/or EPN2 peptide epitope. These genes are well-known and are art-recognized to be annotated according to the following NCBI Gene ID numbers, each of which is available on the World Wide Web at ncbi.nlm.nih.gov/gene: PIEZO1: Gene ID 9780 and NM_001142864.4 and NP_001136336.2 as representative clones; NBEAL1: Gene ID 65065 and NM_001114132.2 and NP_001107604.1, and NM_001378026.1 and NP_001364955.1 as representative clones; NBEAL2: Gene ID 23218 and NM_001365116.2 and NP_001352045.1, and NM_015175.3 and NP_055990.1 as representative clones; EPN2: Gene ID 22905 and NM_001102664.2 and NP_001096134.1, NM_014964.5 and NP_055779.2, and NM_148921.4 and NP_683723.2 as representative clones.

In yet another aspect, a TCR alpha chain and/or beta chain selected from the group consisting of TCR alpha chain and beta chain sequences listed in Table 2, is provided.

In another aspect, an isolated nucleic acid molecule i) that hybridizes, under stringent conditions, with the complement of a nucleic acid encoding a polypeptide selected from the group consisting of polypeptide sequences listed in Table 2, ii) a sequence with at least about 80% homology to a nucleic acid encoding a polypeptide selected from the group consisting of the polypeptide sequences listed in Table 2, and/or iii) ii) a sequence with at least about 80% homology to a nucleic acid encoding listed in Table 2, optionally wherein the isolated nucleic acid molecule comprises 1) a TRAV, TRAJ, and/or TRAC gene or fragment thereof selected from the group of TRAV, TRAJ, and TRAC genes listed in Table 2 and/or 2) a TRBV, TRBJ, and/or TRBC gene or fragment thereof selected from the group of TRBV, TRBJ, and TRBC genes listed in Table 2, is provided.

Numerous embodiments are further provided that may be applied to any aspect encompassed by the present invention and/or combined with any other embodiment described herein. For example, in one embodiment, a nucleic acid is codon optimized for expression in a host cell.

In still another aspect, a vector comprising an isolated nucleic acid described herein, optionally wherein i) the vector is a cloning vector, expression vector, or viral vector and/or ii) the vector comprises a vector sequence listed in Table 3, is provided.

Numerous embodiments are further provided that may be applied to any aspect encompassed by the present invention and/or combined with any other embodiment described herein. For example, in one embodiment, a vector further comprises a nucleic acid sequence encoding CD8α CD8β, a dominant negative TGFβ receptor II (DN-TGFβRII), selectable protein marker, optionally wherein the selectable protein marker is dihydrofolate reductase (DHFR). In another embodiment, a nucleic acid sequence encoding CD8α, CD8β, the DN-TGFβRII, and/or the selectable protein marker is operably linked to a nucleic acid encoding a tag. In still another embodiment, a nucleic acid encoding a tag is at the 5′ upstream of the nucleic acid sequence encoding CD8a, CD8β, the DN-TGFβRII, and/or the selectable protein such that the tag is fused to the N-terminus of CD8α, CD8β, the DN-TGFβRII, and/or the selectable protein marker. In yet another embodiment, a tag is a CD34 enrichment tag. In another embodiment, an isolated nucleic acid described herein, either alone or in combination with a nucleic acid sequence encoding CD8α, CD8β, the DN-TGFβRII, and/or the selectable protein marker are interconnected with an internal ribosome entry site or a nucleic acid sequence encoding a self-cleaving peptide. In still another embodiment, a self-cleaving peptide is P2A, E2A, F2A or T2A.

In yet another aspect, a host cell which comprises an isolated nucleic acid described herein, comprises a vector described herein, and/or expresses a binding protein described herein, optionally wherein the cell is genetically engineered, is provided.

Numerous embodiments are further provided that may be applied to any aspect encompassed by the present invention and/or combined with any other embodiment described herein. For example, in one embodiment, a host cell comprises a chromosomal gene knockout of a TCR gene, an HLA gene, or both. In another embodiment, a host cell comprises a knockout of an HLA gene selected from an α1 macroglobulin gene, α2 macroglobulin gene, α3 macroglobulin gene, β1 microglobulin gene, β2 microglobulin gene, and combinations thereof. In still another embodiment, a host cell comprises a knockout of a TCR gene selected from a TCR α variable region gene, TCR β variable region gene, TCR constant region gene, and combinations thereof. In yet another embodiment, a host cell expresses CD8α, CD8β, a DN-TGFβRII, and/or a selectable protein marker, optionally wherein the selectable protein marker is DHFR, and further optionally wherein the CD8α, CD8β, the DN-TGFβRII, and/or the selectable protein marker is fused to a CD34 enrichment tag. In another embodiment, host cells are enriched using the CD34 enrichment tag. In still another embodiment, a host cell is a hematopoietic progenitor cell, peripheral blood mononuclear cell (PBMC), cord blood cell, or immune cell. In yet another embodiment, an immune cell is a T cell, cytotoxic lymphocyte, cytotoxic lymphocyte precursor cell, cytotoxic lymphocyte progenitor cell, cytotoxic lymphocyte stem cell, CD4⁺ T cell, CD8⁺ T cell, CD4/CD8 double negative T cell, gamma delta (γδ) T cell, natural killer (NK) cell, NK-T cell, dendritic cell, or a combination thereof. In yet another embodiment a T cell is a naive T cell, central memory T cell, effector memory T cell, or a combination thereof. In another embodiment, a T cell is a primary T cell or a cell of a T cell line. In still another embodiment, a T cell does not express or has a lower surface expression of an endogenous TCR. In yet another embodiment, a host cell is capable of producing a cytokine or a cytotoxic molecule when contacted with a target cell that comprises a peptide-MHC (pMHC) complex comprising a MAGEA1 peptide epitope in the context of an MHC molecule. In another embodiment, a host cell is contacted with the target cell in vitro, ex vivo, or in vivo. In still another embodiment, a cytokine is TNF-α, IL-2, and/or IFN-γ. In yet another embodiment, a cytotoxic molecule is perforins and/or granzymes, optionally wherein the cytotoxic molecule is granzyme B. In another embodiment, a host cell is capable of producing a higher level of cytokine or a cytotoxic molecule when contacted with a target cell with a heterozygous expression of MAGEA1. In still another embodiment, a host cell is capable of producing an at least 1.05-fold higher level of cytokine or a cytotoxic molecule. In yet another embodiment, a host cell is capable of killing a target cell that comprises a peptide-MHC (pMHC) complex comprising the MAGEA1 peptide epitope in the context of an MHC molecule. In another embodiment, killing is determined by a killing assay. In still another embodiment, a ratio of the host cell and the target cell in the killing assay is from 20:1 to 1:4. In yet another embodiment, a target cell is a target cell pulsed with 1 μg/mL to 50 μg/mL of MAGEA1 peptide, optionally wherein the target cell is a cell monoallelic for an MHC matched to the MAGEA1 peptide. In another embodiment, a host cell is capable of killing a higher number of target cells when contacted with target cells with a heterozygous expression of MAGEA1, optionally wherein the cell killing is at least 1.05-fold higher. In still another embodiment, a target cell is cell line or a primary cell, optionally wherein the target cell is selected from the group consisting of a HEK293 derived cell line, a cancer cell line, a primary cancer cell, a transformed cell line, and an immortalized cell line. In yet another embodiment, a MAGEA1 immunogenic peptide is as described herein and/or wherein an MHC or MHC-peptide complex is as described herein. In another embodiment, a host cell does not induce T cell expansion, cytokine release, or cytotoxic killing when contact with a target cell that comprises a peptide-MHC (pMHC) complex comprising a PIEZO1, NBEAL1, NBEAL2, and/or EPN2 peptide epitope. In still another embodiment, a host cell does not express MAGEA1 antigen, is not recognized by a binding protein described herein, is not of serotype HLA-A*02, and/or does not express an HLA-A*02 allele.

In another aspect, a population of host cells described herein, is provided.

In still another aspect, a composition comprising a) a binding protein described herein, b) an isolated nucleic acid described herein, c) a vector described herein, d) a host cell described herein, and/or e) a population of host cells described herein, and a carrier, is provided.

In yet another aspect, a device or kit comprising a) a binding protein described herein, b) an isolated nucleic acid described herein, c) a vector described herein, d) a host cell described herein, and/or e) a population of host cells described herein, said device or kit optionally comprising a reagent to detect binding of a), d) and/or e) to a pMHC complex, is provided.

In another aspect, a method of producing a binding protein described herein, wherein the method comprises the steps of: (i) culturing a transformed host cell which has been transformed by a nucleic acid comprising a sequence encoding a binding protein described herein under conditions suitable to allow expression of said binding protein; and (ii) recovering the expressed binding protein, is provided.

In still another aspect, a method of producing a host cell expressing a binding protein described herein, wherein the method comprises the steps of: (i) introducing a nucleic acid comprising a sequence encoding a binding protein described herein into the host cell; and (ii) culturing the transformed host cell under conditions suitable to allow expression of said binding protein, is provided.

In yet another aspect, a method of detecting the presence or absence of a MAGEA1 antigen and/or a cell expressing MAGEA1, optionally wherein the cell is a hyperproliferative cell, comprising detecting the presence or absence of said MAGEA1 antigen in a sample by use of at least one binding protein described herein, at least one host cell described herein, or a population of host cells described herein, wherein detection of the MAGEA1 antigen is indicative of the presence of a MAGEA1 antigen and/or cell expressing MAGEA1, is provided.

Numerous embodiments are further provided that may be applied to any aspect encompassed by the present invention and/or combined with any other embodiment described herein. For example, in one embodiment, at least one binding protein, or at least one host cell, forms a complex with the MAGEA1 peptide in the context of an MHC molecule, and the complex is detected in the form of fluorescence activated cell sorting (FACS), enzyme linked immunosorbent assay (ELISA), radioimmune assay (RIA), immunochemically, Western blot, or intracellular flow assay. In another embodiment, a method further comprises obtaining a sample from a subject.

In another aspect, a method of detecting the level of a disorder characterized by MAGEA1 expression in a subject, comprising: a) contacting a sample obtained from the subject with at least one binding protein described herein, at least one host cell described herein, or a population of host cells described herein; and b) detecting the level of reactivity, wherein the presence or a higher level of reactivity compared to a control level indicates the level of the disorder characterized by MAGEA1 expression in the subject, is provided.

Numerous embodiments are further provided that may be applied to any aspect encompassed by the present invention and/or combined with any other embodiment described herein. For example, in one embodiment, a control level is a reference number. In another embodiment, a control level is a level from a subject without the disorder characterized by MAGEA1 expression.

In still another aspect, a method for monitoring the progression of a disorder characterized by MAGEA1 expression in a subject, the method comprising: a) detecting in a subject sample the presence or level of reactivity between a sample obtained from the subject and at least one binding protein described herein, at least one host cell described herein, or a population of host cells described herein; b) repeating step a) at a subsequent point in time; and c) comparing the level of MAGEA1 or the cell of interest expressing MAGEA1 detected in steps a) and b) to monitor the progression of the disorder characterized by MAGEA1 expression in the subject, wherein an absent or reduced MAGEA1 level or the cell of interest expressing MAGEA1 detected in step b) compared to step a) indicates an inhibited progression of the disorder characterized by MAGEA1 expression in the subject and a presence or increased MAGEA1 level or the cell of interest expressing MAGEA1 detected in step b) compared to step a) indicates a progression of the disorder characterized by MAGEA1 expression in the subject, is provided.

Numerous embodiments are further provided that may be applied to any aspect encompassed by the present invention and/or combined with any other embodiment described herein. For example, in one embodiment, a subject has undergone treatment to treat a disorder characterized by MAGEA1 expression between the first point in time and the subsequent point in time.

In yet another aspect, a method for predicting the clinical outcome of a subject afflicted with a disorder characterized by MAGEA1 expression comprising: a) determining the presence or level of reactivity between a sample obtained from the subject and at least one binding protein described herein, at least one host cell described herein, or a population of host cells described herein; and b) comparing the presence or level of reactivity to that from a control, wherein the control is obtained from a subject having a good clinical outcome; wherein the absence or a reduced level of reactivity in the subject sample as compared to the control indicates that the subject has a good clinical outcome, is provided.

In another aspect, a method of assessing the efficacy of a therapy for a disorder characterized by MAGEA1 expression comprising: a) determining the presence or level of reactivity between a sample obtained from the subject and at least one binding protein described herein, at least one host cell described herein, or a population of host cells described herein, in a first sample obtained from the subject prior to providing at least a portion of the therapy for the disorder characterized by MAGEA1 expression to the subject, and b) determining the presence or level of reactivity between a sample obtained from the subject and at least one binding protein described herein, at least one host cell described herein, or a population of host cells described herein, in a second sample obtained from the subject following provision of the therapy for the disorder characterized by MAGEA1 expression, wherein the absence or a reduced level of reactivity in the second sample, relative to the first sample, is an indication that the therapy is efficacious for treating the disorder characterized by MAGEA1 expression in the subject, and wherein the presence or an increased level of reactivity in the second sample, relative to the first sample, is an indication that the therapy is not efficacious for treating the disorder characterized by MAGEA1 expression in the subject, is provided.

Numerous embodiments are further provided that may be applied to any aspect encompassed by the present invention and/or combined with any other embodiment described herein. For example, in one embodiment, a level of reactivity is indicated by a) the presence of binding and/or b) T cell activation and/or effector function, optionally wherein the T cell activation or effector function is T cell proliferation, killing, or cytokine release. In another embodiment, a T cell binding, activation, and/or effector function is detected using fluorescence activated cell sorting (FACS), enzyme linked immunosorbent assay (ELISA), radioimmune assay (RIA), immunochemically, Western blot, or intracellular flow assay.

In still another aspect, a method of preventing and/or treating a disorder characterized by MAGEA1 expression comprising contacting target cells expressing MAGEA1 with a therapeutically effective amount of a composition comprising cells expressing at least one binding protein described herein, optionally wherein the composition is administered to a subject, is provided.

Numerous embodiments are further provided that may be applied to any aspect encompassed by the present invention and/or combined with any other embodiment described herein. For example, in one embodiment, a cell is an allogeneic cell, syngeneic cell, or autologous cell. In another embodiment, a cell is host cell described herein or a population of host cells described herein. In still another embodiment, a target cell is a cancer cell expressing MAGEA1. In yet another embodiment, a cell composition further comprises a pharmaceutically acceptable carrier. In another embodiment, a cell composition induces an immune response against the target cell expressing MAGEA1 in the subject. In still another embodiment, a cell composition induces an antigen-specific T cell immune response against the target cell expressing MAGEA1 in the subject. In yet another embodiment, an antigen-specific T cell immune response comprises at least one of a CD4⁺ helper T lymphocyte (Th) response and a CD8+ cytotoxic T lymphocyte (CTL) response. In another embodiment, a method further comprises administering at least one additional treatment for the disorder characterized by MAGEA1 expression, optionally wherein the at least one additional treatment for the disorder characterized by MAGEA1 expression is administered concurrently or sequentially with the composition. In still another embodiment, a disorder characterized by MAGEA1 expression is a cancer or relapse thereof, optionally wherein the cancer is selected from the group consisting of melanoma, head & neck cancer, lung cancer, cervical cancer, hepatocellular carcinoma, colorectal cancer, gastrointestinal cancer, breast invasive carcinoma, and bladder urothelial carcinoma. In yet another embodiment, a subject is an animal model of a disorder characterized by MAGEA1 expression and/or a mammal, optionally wherein the mammal is a human, a primate, or a rodent.

### BRIEF DESCRIPTION OF THE DRAWINGS

Certain working examples and figures refer to certain control TCRs, such as a) “Comprator”, also known as “Comparator 1” which corresponds to an Immatics-based TCR described further herein, such as at Table 4, as well as b) “Comparator 2”, which corresponds to a T-Knife-based TCR described further herein, such as at Table 4.

FIGS. 1A and 1B show the identification of 1676 MAGEA1₂₇₈₋₂₈₆-specific TCRs. FIG. 1A shows the co-culture system. Briefly, CD14⁺ monocytes were isolated from PBMCs of HLA-A*02:01 healthy donors on day-4 and differentiated to mature DCs. On day-1, naive CD8 T cells were isolated from autologous PBMCs and rested overnight. Co-culture of naive CD8 T cells and DCs was performed following 3 hours pulsing of DCs with 1 μg/mL MAGEA1₂₇₈₋₂₈₆ peptide as part of multiplexed screens, followed by an 11-day cell expansion phase. FIG. 1B shows the screening process. Dextramer staining was performed with HLA-A*02:01-restricted MAGEA1₂₇₈₋₂₈₆ (KVLEYVIKV (SEQ ID NO: 1)) dextramer to identify clones and to sort MAGEA1₂₇₈₋₂₈₆-specific cells. Sequencing of isolated T cells and pairing of TCR alpha and beta chains was performing using the 10× Genomics platform.

FIGS. 2A and 2B show the selection of 30 out of 500 TCRs by multiple rounds of VAYG screens for functional assessment. FIG. 2A shows T cell cytotoxicity of NCIH1703 (HLA-A*02:01+MAGEA1) targets at E:T of 5:1. FIG. 2B shows that 30 out of 500 TCRs were selected by multiple rounds of VAYG screens for functional assessment.

FIGS. 3A-3E show results of selecting MAGEA1₂₇₈₋₂₈₆ TCRs based on expression and cytotoxic function. FIG. 3A shows expression of MAGEA1 TCRs on the surface of engineered T cells. FIG. 3B shows T cell cytotoxicity of NCIH1703 (HLA-A*02:01+MAGEA1) targets at an effector cell to T cell (E:T) ratio of 4:1. FIG. 3C shows T cell cytotoxicity of Hs936T (HLA-A*02:01+MAGEA1) targets at E:T of 4:1. FIG. 3D shows T cell cytotoxicity of A375 (HLA-A*02:01+MAGEA1) targets at E:T of 4:1. FIG. 3E shows T cell cytotoxicity of HEK293T (HLA-A*02:01-MAGEA1) targets at E:T of 4:1.

FIGS. 4A-4F show the functional evaluation of MAGEA1₂₇₈₋₂₈₆ TCRs. FIG. 4A shows expression of MAGEA1₂₇₈₋₂₈₆ TCR 1134 and TCR 1479 on surface of engineered T cells. FIG. 4B shows TCR 1134 cytokine production in response to HLA-A*02:01+MAGEA1^{+/-} targets. FIG. 4C shows TCR 1479 cytokine production in response to HLA-A*02:01+MAGEA1^{+/-} targets. FIG. 4D shows T cell cytotoxicity of NCIH1703 (HLA-A*02:01+MAGEA1) targets at E:T of 4:1. FIG. 4E shows T cell cytotoxicity of Hs936T (HLA-A*02:01+MAGEA1) targets at E:T of 4:1. FIG. 4F shows T cell cytotoxicity of HEK293T (HLA-A*02:01-MAGEA1) targets at E:T of 4:1.

FIG. 5 shows the peptide dilution curve for TCR MAGE-A1-1479.

FIG. 6 shows the identification of the putative off-targets for TCR MAGE-A1-1479 by the proprietary genome-wide screen.

FIG. 7 shows that TCR MAGE-A1-1479 shows no alloreactivity to 109/110 MHCs tested.

FIGS. 8A-8D show that MAGE-A1-1479 shows no reactivity to healthy human primary cells.

FIG. 9 shows pMHC dose-dependent function of Process-representative TSC-204-A0201 TCR-T cells. T2 cells were pulsed with various concentrations of the MAGE-A1 peptide and co-cultured with three batches of TSC-204-A0201 process-representative TCR-T cells. The figure shows the IFN-γ secretion as a read-out for the TCR-T cell reactivity to various cognate peptide doses. Note: IFN-γ was normalized to 0% based on the smallest mean in each data set (n=3) and 100% based on the largest mean in each data set. Results are presented as percentages. A nonlinear regression fit was used to display a “normalized response” model.

FIGS. 10A-10E show TSC-204-A0201 TCR-T cells secrete granzyme B and the inflammatory cytokines IFN-γ, TNF α and IL-2 in a target-dependent manner. Granzyme B and inflammatory cytokines were quantified in the supernatant of co-cultures (E:T 1:1) of TSC-204-A0201 TCR-T cells (orange (i.e., left column of each pair) or untransfected (UTF) control T cells from matched donors (grey) with the indicated cell lines using automated ELISA (ELLA). Dashed lines indicate cytokine and Granzyme B levels of unstimulated TCR-T cells, i.e., TCR-T cells cultured without cancer cell lines. Note that for some conditions, the baseline was too low to be displayed on the graph. Furthermore, for the UTF control, some values were beneath the detection level (indicated with asterisks). Note that different Y axis scales were used to depict strong (FIGS. 10A-10C) and weak or absent (FIGS. 10D and 10E) cytokine and granzyme B responses.

FIGS. 11A-11E show that both helper (CD4⁺) T cells and cytotoxic (CD4⁻) T cells in TSC-204-A0201 proliferate in a target-dependent manner. TSC-204-A0201 TCR-T cells were labeled with CTV dye and were co-cultured for 3.5 days with the indicated cancer cell lines. Subsequently, CTV dye dilution (indicative of proliferation) was assessed within the transduced fraction (i.e., CD34⁺) of TSC-204-A0201 TCR-T cells, and within helper T cells (CD34⁺CD4⁺) and cytotoxic T cells (CD34⁺CD4⁻) (orange (darkest shade)). The percentage of TSC-204-A0201 T cells that cycled once, twice or three or more times is indicated. Proliferation was also assessed in untransfected (UTF) control T cells from matched donors (grey). For UTF controls, the proliferation was assessed in the CD34⁻CD4⁺ and CD34⁻CD4⁻ populations. The three dashed lines represent baseline proliferation from the three donor-matched UTF controls as follows: PD269 (low), PD272 (middle) and PD274 (high).

FIGS. 12A and 12B show that TSC-204-A0201 TCR-T cells display potent and selective killing activity. FIG. 12A shows results of three batches of process-representative TSC-204-A0201 TCR-T cells (orange (shaded), circle) and untransfected (UTF) control T cells from matched donor (gray, circle) as analyzed in the Incucyte®-based cytotoxicity assay for their cytotoxicity potential against the indicated target cell lines. Effector TCR-T cells and target cells were co-cultured across a range of effector to target ratios (E:T ranging from 10:1 to 0.3:1) and the growth of the target cells was measured over 72 hours. Data presented were obtained with the TSC-204-A0201 TCR-T cells and UTF from the batch PD272 and are representative of the data obtained with all 3 batches of process-representative material tested. Target cells cultured alone are displayed as a negative control (black, triangle). FIG. 14B shows cytotoxic activity of the three batches of process-representative TSC-204-A0201 TCR-T cells over 72 hours and is summarized as the normalized target cell growth calculated as the ratio of the area under the curve (AUC) for target cell growth co-cultured with the indicated batch of TSC-204-A0201 at the E:T of 5 to 1 for 72 hours over the area under the curve of the target cell growth under the same co-culture conditions with matching UTF control T cells.

FIGS. 13A-13D show that DN-TGFβRII expression confers resistance to TGFβ-mediated suppression of target induced cytokine and Granzyme B secretion. After 20 hour preincubation with 0 or 5 ng/mL TGFβ, TSC-204-A0201 TCR-T cells were incubated consecutively with two rounds of target cells: to allow depletion of preformed cytokine mRNA and granzyme B protein. TCR-T cells were first incubated for 20 hour with HLA-A*02:01-positive and MAGE-A1-positive U266B1 cells. TCR-T cells were then spun down, the supernatant was discarded, and the TCR-T cells were incubated for an additional 20 hour with a second round of target cells as indicated in the figure, either U266B1 (HLA-A*02:01-positive and MAGE-A1-positive) or LOUCY (HLA-A*02:01-positive, MAGE-A1-negative). TGFβ concentration was maintained at 0 or 5 ng/mL throughout the two rounds of co-culture. Cytokine (IFN-γ, TNF-α, and IL-2) and granzyme B secretion was evaluated with an automated ELISA (ELLA) at the end of the second round of co-culture. DN-TGFβRII-negative TSC-204-A0201 TCR-T cells (D5662), shown in black (i.e., left two columns), correspond to process-similar TCR-T cells produced for Example 15 and are included here as control for TGFβ inhibition. Shown in orange (i.e., right two columns) are cytokine and granzyme B secretion of three batches of process-representative TCR-T cells (batches PD269, PD272 and PD274).

FIG. 14 shows inoculation, dosing, and analyses schedule for animals in groups 1-5.

FIG. 15 shows inoculation, dosing, and sampling schedule for animals in groups 6-7.

FIGS. 16A and 16B show in vivo efficacy of TSC-204-A0201 TCR-T cells. NCG mice were inoculated s.c. with U266B1. Once tumor engraftment was successful (tumors reaching 100 mm³ on average, 21 days post inoculation), animals were randomized into different treatment groups. Animals then received two i.v. injections of process-representative TSC-204-A0201 TCR-T cells (2 batches tested, PD269 and PD272), of untransfected (UTF) control T cells from matched donors, or of vehicle (PBS) on day 1 and 8 of the study (arrow heads). The total number of cells injected corresponded to 2E7 CD34⁺ for each batch. FIG. 16A represents mean tumor volume over time for the different groups. FIG. 16B shows individual mouse tumor growth per group over time, separating the two batches of TSC-204-A0201.

FIG. 17 shows average body weight evolution over time across the different groups. NCG mice were inoculated s.c. with U266B1. Once tumor engraftment was successful (tumors reaching 100 mm³ on average, 21 days post inoculation), animals were randomized into different treatment groups. Animals then received two i.v. injections of process-representative TSC-204-A0201 TCR-T cells (2 batches tested, PD269 and PD272), of untransfected (UTF) control T cells from matched donors, or of vehicle (PBS) on day 1 and 8 of the study (arrow heads). Animals' body weight (BW) were measured every 3 days after initiation of treatment. Average body weight per treatment group is shown.

FIGS. 18A and 18B show T cell persistence in peripheral blood. Blood was sampled at the indicated days post initiation of treatment and analyzed by flow cytometry to identify mouse (mCD45) and human (hCD45) cells. Human cells were further analyzed for CD34 positivity. Graphs show percentages of (FIG. 18A) human CD45⁺ immune cells and (FIG. 21B) human immune cells positive for CD34 (FIG. 18B) in the blood of mice dosed with TSC-204-A0201 TCR-T cells from PD269 and PD272 batches.

FIG. 19 shows steps and timelines of the cytokine assay to test off-tumor reactivity of TSC-204-A0201 TCR-T cells.

FIG. 20 shows the expression of MAGE-A1 and putative off-targets of the therapeutic TCR used in TSC-204-A0201 TCR-T cells in cancer cell lines. NA was extracted from the cancer cell lines and sequenced. Heat maps show TPM (transcripts per million) calculated from the counts. The scale used in RNAseq heatmaps has TPM values of zero set to white and values above zero follow a continuous pigment density scale up to 100 TPM.

FIGS. 21A-21C show that TSC-204-A0201 TCR-T cells show no reactivity to HLA-A*02:01+ cancer cell lines expressing off-targets of the TCR. SC-204-A0201 TCR-T cells and donor-matched UTF cells were co-cultured with a panel of cancer cell lines and supernatants were evaluated for levels of IFN-γ as a measure of T cell reactivity.

FIG. 22 shows the expression of MAGE-A1 and off-targets of the therapeutic TCR used in TSC-204-A0201 TCR-T cells in primary cells and iPSC-derived cells. NA was extracted from the cells and sequenced. TPM (transcripts per million) was calculated from the counts, and average TPM was calculated for the replicates of the same cell type. The colorscale used in RNAseq heatmaps has TPM values of zero set to white and values above zero follow a continuous pigment density scale up to 100 TPM.

FIGS. 23A-23C provide representative graphs indicating that TSC-204-A0201 TCR-T cells show no reactivity to HLA-A*02:01+ primary cells. TSC-204-A0201 TCR-T cells and donor-matched UTF cells were co-cultured with a panel of primary cells and supernatants were evaluated for levels of IFN-γ as a measure of T cell reactivity.

FIG. 24 shows steps and timelines of the oncogenicity assay used to evaluate the cytokine-dependency of proliferating T cells.

FIG. 25 shows results of T cell viability analyses. Data show the normalized (using Count Bright beads) numbers of viable (eFlour 660-negative) UTF-T (grey bars; right bar of each pair) and TSC-204-A0201 TCR-T (orange bars; left bar of each pair) cells from donor PD268, donor PD269, and donor PD272 after 5 days of in-vitro culture in the absence (−) or presence (+) of cytokines and ImmunoCult. The dotted line represents the initial numbers of cells (80,000) used in this assay. **** p≤0.0001; *** p≤0.001; ** p≤0.01; * p≤0.05; ‘ns’ means not significant, p>0.05.

FIG. 26 shows results of T cell proliferation analyses. Data show the normalized (using Count Bright beads) numbers of proliferating (dividing) UTF-T (grey bars; right bar of each pair) and TSC-204-A0201 TCR-T (orange bars; left bar of each pair) cells from donor PD268, donor PD269, and donor PD272 after 5 days of in-vitro culture in the absence (−) or presence (+) of cytokines or ImmunoCult. **** p≤0.0001; *** p≤0.001; ** p≤0.01; * p≤0.05; ‘ns’ means not significant, p>0.05.

FIG. 27 shows percentages of dividing cells from proflieration analyses. Data show the percent (%) of proliferating (dividing) UTF-T (grey bars; right bar of each pair) and TSC-204-A0201 TCR-T (orange bars; left bar of each pair) gated on viable cells from donor PD268, donor PD269, and donor PD272 after 5 days of culturing in the absence (−) or presence (+) of cytokines or ImmunoCult. **** p≤0.0001; *** p≤0.001; ** p≤0.01; * p≤0.05; ‘ns’ means not significant, p>0.05.

FIG. 28 shows MAGE-A1 expression in 48 normal human organs.

FIG. 29 shows MAGE-A1 expression in 24 different brain tissues.

FIG. 30 shows a representative schematic of the expression vector used to engineer TSC-204-A0201 TCR-T cells.

FIGS. 31A and 31B show that DN-TGFβRII expression confers resistance to TGFβ-mediated suppression of target induced cytokine and granzyme B secretion. After 20 hour preincubation with 0 or 5 ng/mL TGFβ, TCR-T cells were incubated consecutively with two rounds of target cells: to allow depletion of preformed cytokine mRNA and Granzyme B protein, TCR-T cells were first incubated for 20 hour with MAGE-A1-positive U266B1 cells. TCR-T cells were then spun down, the supernatant was discarded, and the TCR-T cells were incubated for an additional 20 hour with a second round of target cells, either MAGE-A1 positive U266B1 cells (FIG. 31A) or MAGE-A1 negative LOUCY cells (FIG. 31B). TGFβ concentration was maintained at 0 or 5 ng/mL throughout the two rounds of co-culture. Cytokine (IFN-γ, TNF-α, and IL-2) and granzyme B secretion was evaluated with an automated ELISA (ELLA) at the end of the second round of co-culture. TCR-T cells engineered from two donors (D5662 and D6418) were evaluated. Furthermore, for each donor, TCR-T cells expressing DN-TGFβRII were compared with TCR-T cells lacking DN-TGFβRII expression (see legend below graphs). Cytokine and granzyme B responses of TSC-204-A0201 TCR-T cells are depicted.

FIGS. 32A-32F show that TSC-204-A0201 TCR-T cells expressing DN-TGFβRII are protected from TGFβ-mediated inhibition of T cell expansion and proliferation. T cells from two donors (D5662 and D6418) were engineered with the clinical TSC-204-A0201 vector, or a vector identical to the clinical vector but lacking the DN-TGFβRII gene. DN-TGFβRII positive and DN-TGFβRII negative TSC-204-A0201 TCR-T cells were then labeled with Cell Trace Violet dye (CTV) and were co-cultured for 3.5 days with cancer cell lines expressing HLA-A*02:01 and MAGE-A1 (FIGS. 32A and 32B, SW1271; FIGS. 32C and 32D, HS936T) or a cell line expressing HLA-A*02:01 but negative for MAGE-A1 (FIGS. 32E and 32F, LOUCY). TGFβ was added to the co-cultures at a final concentration of 0 or 5 ng/mL. At the end of the co-culture, cell counts (left hand panels) and proliferation (right hand panels) were assessed by flow cytometry. Based on CTV dye dilution, the total percentage of proliferating cells was quantified, as well as the percentage of cells that had undergone one, two or three or more cell cycles, as indicated in the legend below the proliferation panels.

FIGS. 33A and 33B show co-culture of TSC-204-A0201 TCR-T cells with U266B1 cells. T cells from two donors (D5662 and D6418) were engineered with the DN-TGFβRII positive or DN-TGFβRII negative TSC-204-A0201 TCR-T cells. DN-TGFβRII positive and DN-TGFβRII negative TCR-T cells were then labeled with CellTrace® Violet dye (CTV) and were co-cultured for 3.5 days with the MAGE-A1, HLA-A*02:01 positive cell line U266B1. TGFβ was added to the co-cultures at a final concentration of 0 or 5 ng/ml. At the end of the co-culture, cell counts (left hand panels) and proliferation (right hand panels) of TSC-204-A0201 TCR-T cells were assessed by flow cytometry. Based on CTV dye dilution, the total percentage of proliferating cells was quantified, as well as the percentage of cells that had undergone one, two or three or more cell cycles, as indicated in the legend below the proliferation panels.

FIG. 34 shows that adding DN-TGFbRII to TCR-T cells enables target-dependent proliferation in the presence of TGFb.

FIG. 35 shows that expression of DN-TGFβRII has little effect on the cytotoxic activity of TSC-204-A0201 TCR-T cells. An Incucyte®-based cytotoxicity assay was performed with TSC-204-A0201 TCR-T cells expressing DN-TGFβRII or not (orange circles (light shade), DN-TGFβRII-positive; black circles, DN-TGFβRII-negative). MAGE-A1-positive target cells (SW1271, HS936T or AU565) were co-cultured with TCR-T cells at variable effector to target ratios (0.04-20). TGFβ was added at a final concentration of 0 or 5 ng/ml and the target cell growth was measured for 72 hours. Graphs depict area under the curve (AUC) plotted versus E:T ratios.

FIG. 36 shows that DN-TGFβRII enhances duration of activity in vivo.

FIG. 37 shows a map of the pNVVD 136 (i.e., pNVVD136_TSC-204-A02_TCR-1479_MSCV-TCR-1479-CD8-EF1α-dnTGFbRII-DHFR) vector. CD: cluster of differentiation RNA-OUT: anti-sense RNA against the bacterial levansucrase encoded by sacB. SV: simian virus TCR: T Cell Receptor, ITR: inverted terminal repeat, QBend: Mouse anti Human CD34 antibody, dnTGFbRII: Dominant-negative TGF beta Receptor II, DHFR: Dihydrofolate reductase selection marker.

FIG. 38 shows a map of the pNVVD 166 (i.e., pNVVD166_TSC-204-A02_TCR-1479_MSCV-TCR-1479-CD8-EF1a-DHFR) vector. CD: cluster of differentiation RNA-OUT: anti-sense RNA against the bacterial levansucrase encoded by sacB. SV: simian virus TCR: T Cell Receptor, ITR: inverted terminal repeat, QBend: Mouse anti Human CD34 antibody, DHFR: Dihydrofolate reductase selection marker.

For any figure showing a bar histogram, curve, or other data associated with a legend, the bars, curve, or other data presented from left to right for each indication correspond directly and in order to the boxes from top to bottom, or from left to right, of the legend unless indicated otherwise.

### DETAILED DESCRIPTION OF THE INVENTION

The present invention is based, at least in part, on the discovery of MAGEA1 immunogenic peptides (e.g., those comprising or consisting of sequences listed in Table 1), binding proteins (e.g., those having sequences listed in Table 2) that recognize MAGEA1 antigens, and uses thereof. A systematic, comprehensive survey was carried out to map the precise T cell targets recognized by an initial pool of T cells of interest.

Accordingly, the present invention relates, in part, to the identified epitopes (immunodominant peptides) of therapeutically relevant MAGEA1 protein and related compositions (e.g., immunodominant peptides, vaccines, and the like), compositions comprising immunogenic peptides alone or with MHC molecules, stable MHC-peptide complexes, methods of diagnosing, prognosing, and monitoring immune responses to disorders characterized by MAGEA1 expression, and methods for preventing and/or treating disorders characterized by MAGEA1 expression. The present invention also relates, in part, to identified binding proteins (e.g., TCRs), host cells expressing binding proteins (e.g., TCRs), compositions comprising binding proteins (e.g., TCRs) and host cells expressing binding proteins (e.g., TCRs), methods of diagnosing, prognosing, and monitoring T cell response to cells expressing MAGEA1, and methods for preventing and/or treating disorders characterized by MAGEA1 expression.

### I. Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element. In addition, references to a table provided herein encompass all sub-tables of the table unless otherwise indicated.

The term “administering” means providing a pharmaceutical agent or composition to a subject, and includes, but is not limited to, administering by a medical professional and self-administering. This involves the physical introduction of a composition comprising a therapeutic agent to a subject, using any of the various methods and delivery systems known to those skilled in the art. In some embodiments, routes of administration for binding proteins described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, a binding protein described herein may be administered via a non-parenteral route, such as a topical, epidermal or mucosal route of administration, for example, intranasally, orally, vaginally, rectally, sublingually or topically. Administering may also be performed, for example, once, a plurality of times, and/or over one or more extended periods.

As used herein, the term “antigen” refers to any natural or synthetic immunogenic substance, such as a protein, peptide, or hapten. An antigen may be a MAGEA1 antigen, or a fragment thereof, against which protective or therapeutic immune responses are desired. An “epitope” is the part of the antigen bound by a natural or synthetic substance.

The term “adjuvant” as used herein refers to substances, which when administered prior, together or after administration of an antigen accelerates, prolong and/or enhances the quality and/or strength of an immune response to the antigen in comparison to the administration of the antigen alone. Adjuvants can increase the magnitude and duration of the immune response induced by vaccination.

The term “antibody” as used to herein includes whole antibodies and any antigen binding fragments (i.e., “antigen-binding portions”) or single chains thereof. An “antibody” refers, in one embodiment, to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. In certain naturally occurring antibodies, the heavy chain constant region is comprised of three domains, CH1, CH2 and CH3. In certain naturally occurring antibodies, each light chain is comprised of a light chain variable region (abbreviated herein as V_{L}) and a light chain constant region. The light chain constant region is comprised of one domain, CL. The V_{H} and V_{L} regions may be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen presenting cell” or “APC” includes professional antigen presenting cells (e.g., B lymphocytes, monocytes, dendritic cells, Langerhans cells), as well as other antigen presenting cells (e.g., keratinocytes, endothelial cells, astrocytes, fibroblasts, and oligodendrocytes).

The term “antigen-binding portion” of a binding protein, such as a TCR, as used herein, refers to one or more portions of a TCR that retain the ability to bind (e.g., specifically and/or selectively) to an antigen (e.g., a MAGEA1 antigen). Such portions are, for example, between about 8 and about 1,500 amino acids in length, suitably between about 8 and about 745 amino acids in length, suitably about 8 to about 300, for example about 8 to about 200 amino acids, or about 10 to about 50 or 100 amino acids in length. It has been shown that the antigen-binding function of a TCR can be performed by fragments of a full-length TCR. Examples of binding portions encompassed within the term “antigen-binding portion” of a TCR, include (i) a Fv fragment consisting of the V_{α} and V_{β} domains of a TCR, (ii) an isolated complementarity determining region (CDR) or (iii) a combination of two or more isolated CDRs which may optionally be joined by a synthetic linker. Furthermore, although V_{α} and V_{β}, are coded by separate genes, they may be joined, using recombinant methods, by a synthetic linker that enables them to be made as a single protein chain in which the V_{α} and V_{β} regions pair to form monovalent molecules (known as single chain TCR (scTCR)). Such single chain TCRs are also intended to be encompassed within the term “antigen-binding portion” of a TCR. These TCR fragments can be obtained using conventional techniques known to those with skill in the art, and the fragments are screened for utility in the same manner as are complete binding proteins. Antigen-binding portions may be produced by recombinant DNA techniques, or by enzymatic or chemical cleavage of intact immunoglobulins.

The terms “complementarity determining region” and “CDR” are synonymous with “hypervariable region” or “HVR” and are known in the art to refer to non-contiguous sequences of amino acids within certain binding proteins, such as TCR variable regions, which confer antigen specificity and/or binding affinity. For TCRs, in general, there are three CDRs in each α-chain variable region (αCDR1, αCDR2, and αCDR3) and three CDRs in each β-chain variable region (βCDR1, βCDR2, and βCDR3). CDR3 is believed to be the main CDR responsible for recognizing processed antigen. CDR1 and CDR2 mainly interact with the MHC.

The term “body fluid” refers to fluids that are excreted or secreted from the body as well as fluids that are normally not excreted or secreted from the body (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit). In some embodiments, the body fluid comprises immune cells, optionally wherein the immune cells are cytotoxic lymphocytes such as cytotoxic T cells and/or NK cells, CD4+ T cells, and the like.

The term “coding region” refers to regions of a nucleotide sequence comprising codons that are translated into amino acid residues, whereas the term “non-coding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is anti-parallel to the first region if the residue is thymine or uracil. Similarly, it is known that a cytosine residue of a first nucleic acid strand is capable of base pairing with a residue of a second nucleic acid strand which is anti-parallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, and, in other embodiments, at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or any range in between, inclusive, such as at least about 80%-100%, of the nucleotide residues of the first portion are capable of base pairing with nucleotide residues in the second portion. In some embodiments, all nucleotide residues of the first portion are capable of base pairing with nucleotide residues in the second portion.

As used herein, the term “costimulate” with reference to activated immune cells includes the ability of a costimulatory molecule to provide a second, non-activating receptor mediated signal (a “costimulatory signal”) that induces proliferation or effector function. For example, a costimulatory signal may result in cytokine secretion, e.g., in a T cell that has received a T cell-receptor-mediated signal. Immune cells that have received a cell-receptor mediated signal, e.g., via an activating receptor are referred to herein as “activated immune cells.”

“CD3” is known in the art as a multi-protein complex of six chains (see, Abbas and Lichtman, Cellular and Molecular Immunology (9^{th} Edition) (2018); Janeway et al. (Immunobiology) (9^{th} Edition) (2016)). In mammals, the complex comprises a CD3γ chain, a CD3δ chain, two CD3ε chains, and a homodimer of CD3ζ chains. The CD3γ, CD3δ, and CD3ε chains are related cell surface proteins of the immunoglobulin superfamily containing a single immunoglobulin domain. The transmembrane regions of the CD3γ, CD3δ, and CD3ε chains are negatively charged, which is a characteristic that is believed to allow these chains to associate with positively charged regions or residues of T cell receptor chains. The intracellular tails of the CD3γ, CD3δ, and CD3ε chains each contain a single conserved motif known as an immunoreceptor tyrosine-based activation motif or IT AM, whereas each CD3ζ chain has three ITAMs. Without wishing to be bound by theory, it is believed that the IT AMs are important for the signaling capacity of a TCR complex. CD3 used in accordance with the present invention may be from various animal species, including human, mouse, rat, or other mammals.

A “component of a TCR complex,” as used herein, refers to a TCR chain (i.e., TCRα, TCRβ, TCRγ or TCRδ), a CD3 chain (i.e., CD3γ, CD3δ, CD3ε or CD3ζ), or a complex formed by two or more TCR chains or CD3 chains (e.g., a complex of TCRα and TCRβ, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains).

Comparator T-cell receptor” refers to at least one benchmark T-cell receptor (e.g., Immatics-based or T-Knife-based) that has been reported in the state of the art, such as U.S. Pat. No. 10,874,731 (Immatics) and Obenaus et al. (2014) Nat. Biotechnol. 33:402-407. In some embodiments, “Comparator 1”, also referred to simply as “Comparator”, is an Immatics R37P1C9 TCR-based TCR from U.S. Pat. No. 10,874,731. Engineered versions of such parental sequences were used in the working examples and sequences of such engineered versions are set forth in Table 4. In some embodiments, “Comparator 2” refers to a T-Knife-T1367 TCR-based TCR from Obenaus et al. (2014) Nat. Biotechnol. 33:402-407. Engineered versions of such parental sequences were used in the working examples and sequences of such engineered versions are set forth in Table 4. In some embodiments, the comparator T-cell receptor has sequences set forth in Table 4.

The term “chimeric antigen receptor” or “CAR” refers to a fusion protein that is engineered to contain two or more amino acid sequences linked together in a way that does not occur naturally or does not occur naturally in a host cell, which fusion protein can function as a receptor when present on a surface of a cell. CARs encompassed by the present invention include an extracellular portion comprising an antigen-binding domain (i.e., obtained or derived from an immunoglobulin or immunoglobulin-like molecule, such as a TCR specific for a MAGEA1 antigen, a single chain TCR-derived binding protein, an scFv derived from an antibody, an antigen binding domain derived or obtained from a killer immunoreceptor from an NK cell, and the like) linked to a transmembrane domain and one or more intracellular signaling domains (such as an effector domain, optionally containing co-stimulatory domain(s)) (see, e.g., Sadelain et al. (2013) Cancer Discov. 3:388; see also Harris and Kranz (2016) Trends Pharmacol. Sci. 37:220; Stone et al. (2014) Cancer Immunol. Immunother. 63:1163).

As used herein, the term “cytotoxic T lymphocyte (CTL) response” refers to an immune response induced by cytotoxic T cells. CTL responses are mediated primarily by CD8⁺ T cells.

The term “consisting essentially of is not equivalent to “comprising” and refers to the specified materials or steps of a claim, or to those that do not materially affect the basic characteristics of a claimed subject matter. For example, a protein domain, region, or module (e.g., a binding domain, hinge region, linker module) or a protein (which may have one or more domains, regions, or modules) “consists essentially of a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

The term “determining a suitable treatment regimen for the subject” is taken to mean the determination of a treatment regimen (i.e., a single therapy or a combination of different therapies that are used for the prevention and/or treatment of the viral infection in the subject) for a subject that is started, modified and/or ended based or essentially based or at least partially based on the results of the analysis according to the present invention. One example is starting an adjuvant therapy after surgery whose purpose is to decrease the risk of recurrence, another would be to modify the dosage of a particular chemotherapy. The determination can, in addition to the results of the analysis according to the present invention, be based on personal characteristics of the subject to be treated. In most cases, the actual determination of the suitable treatment regimen for the subject will be performed by the attending physician or doctor.

The term “dominant negative TGFβ receptor” or “DN-TGFβR” refers to a transforming growth factor (TGF) beta receptor variant or mutant that provides resistance to TGFβ signaling. There are five type II receptors (activation receptors) and seven type I receptors (signaling propagation receptors). The active TGF receptor is a heterotetramer consisting of two TGF β receptors I (TGFβRI) and two TGF β receptors II (TGFβRII). In some embodiments, the DN-TGFβR is a DN-TGFβRII (i.e., a TGF beta receptor II variant or mutant). In some embodiments, resistance is to the suppressive effect of TGFβ signaling on an immune cell, such as a T cell, which TGFβ may be produced by cancer cells or by other immune cells within a cellular environment, such as by stromal cells, macrophages, myeloid cells, epithelial cells, natural killer cells, and the like. TGFβ signaling inhibitors are well-known in the art and include, without limitation, mutant TGFβ that sequesters receptors and thereby inhibits signaling, antibodies that bind to TGFβ and/or TGFβ receptors (e.g., lerdelimumab, metelimumab, fresolimumab, and the like), soluble TGFβ-binding proteins such as portions of TGFβ receptors that sequester TGFβ (e.g., TGFβRII-Fc fusion proteins) or other binders, such as beta-glycans. Any and all known TGFβ signaling inhibitors may be used instead of or in addition to DN-TGFβR (e.g., DN-TGFβRII) described herein. In some embodiments, a DN-TGFβR lacks an intracellular portion required for TGFβ-mediated signaling, such as the entire intracellular domain, a kinase signaling domain, etc. DN-TGFβR constructs are well-known in the art (see representative, non-limiting embodiments at Brand et al. (1993) J. Biol. Chem. 268:11500-11503; Weiser et al. (1993) Mol. Cell Biol. 13:7239-7247; Bollard et al. (2002) Blood 99: 3179-3187; PCT Publ. WO 2009/152610; PCT Publ. WO 2017/156484; Kloss et al. (2018) Mol. Ther. 26:1855-1866; PCT Publ. WO. 2019/089884; PCT Publ. WO 2020/042647; and PCT Publ. WO 2020/042648.

As used herein, a “hematopoietic progenitor cell” is a cell that can be derived from hematopoietic stem cells or fetal tissue and is capable of further differentiation into mature cells types (e.g., immune system cells). Exemplary hematopoietic progenitor cells include those with a CD24^{Lo} Lin-CD117⁺ phenotype or those found in the thymus (referred to as progenitor thymocytes).

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. In some embodiments, the first region comprises a first portion and the second region comprises a second portion, whereby, at least about 50%, and, in other embodiments, at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or any range in between, inclusive, such as at least about 80%-100%, of the nucleotide residue positions of each of the portions are occupied by the same nucleotide residue. In some embodiments, all nucleotide residue positions of each of the portions are occupied by the same nucleotide residue.

The term “hyperproliferative disorder characterized by expression of a MAGEA1 antigen” can be any hyperproliferative disorder where the MAGEA1 antigen is present in a MHC (e.g., HLA) complex expressed by at least some hyperproliferating cells in the subject. Examples of hyperproliferative disorders characterized by MAGEA1: HLA complexes include solid malignancies, such as those described in detail infra.

The term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages.

An increased ability to stimulate an immune response or the immune system, can result from an enhanced agonist activity of T cell costimulatory receptors and/or an enhanced antagonist activity of inhibitory receptors. An increased ability to stimulate an immune response or the immune system may be reflected by a fold increase of the EC₅₀ or maximal level of activity in an assay that measures an immune response, e.g., an assay that measures changes in cytokine or chemokine release, cytolytic activity (determined directly on target cells or indirectly via detecting CD107a or granzymes) and proliferation. The ability to stimulate an immune response or the immune system activity may be enhanced by at least 10%, 20%, 30%, 40%, 50%, 60%, 70%, 80%, 90%, 100%, 110%, 120%, 130%, 140%, 150%, 160%, 170%, 180%, 190%, 200%, 250%, 300%, 350%, 400%, 500%, or more.

The term “immunotherapeutic agent” may include any molecule, peptide, antibody or other agent which can stimulate a host immune system to generate an immune response to a cancer cell in the subject. Various immunotherapeutic agents are useful in the compositions and methods described herein.

The term “immune cell” refers to any cell of the immune system that originates from a hematopoietic stem cell in the bone marrow, which gives rise to two major lineages: a myeloid progenitor cell (which give rise to myeloid cells such as monocytes, macrophages, dendritic cells, megakaryocytes and granulocytes); and a lymphoid progenitor cell (which give rise to lymphoid cells such as T cells, B cells and natural killer (NK) cells). Exemplary immune system cells include a CD4⁺ T cell, a CD8⁺ T cell, a CD4 CD8 double negative T cell, a gd T cell, a regulatory T cell, a natural killer cell, and a dendritic cell. Macrophages and dendritic cells may be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

An “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the binding protein, antibody, polypeptide, peptide or fusion protein is derived, or substantially free from chemical precursors or other chemicals when chemically synthesized. The language “substantially free of cellular material” includes preparations of a biomarker polypeptide or fragment thereof, in which the protein is separated from cellular components of the cells from which it is isolated or recombinantly produced. In one embodiment, the language “substantially free of cellular material” includes preparations of a biomarker protein or fragment thereof, having less than about 30% (by dry weight) of non-biomarker protein (also referred to herein as a “contaminating protein”), or, in some embodiments, less than about 25%, 20%, 15%, 10%, 5%, 1%, or less, or any range in between inclusive, such as less than about 1% to 5%, of non-biomarker protein. When binding protein, antibody, polypeptide, peptide or fusion protein or fragment thereof, e.g., a biologically active fragment thereof, is recombinantly produced, it may be substantially free of culture medium, i.e., culture medium represents less than about 20%, 15%, 10%, 5%, 1%, or less, or any range in between inclusive, such as less than about 1% to 5%, of the volume of the protein preparation.

As used herein, the term “isotype” refers to the antibody class (e.g., IgM, IgG1, IgG2C, and the like) that is encoded by heavy chain constant region genes.

As used herein, the term “K_{D}” is intended to refer to the dissociation equilibrium constant of a particular binding protein-antigen interaction. The binding affinity of binding proteins encompassed by the present invention may be measured or determined by standard binding protein-target binding assays, for example, competitive assays, saturation assays, or standard immunoassays, such as ELISA or RIA. A relatively lower Kd value indicates a relatively higher binding affinity (e.g., Kd values of less than or equal to about 5×10⁻⁴ M (500 uM) include a Kd value of 1×10⁻⁴ M (100 μM) and a 100 μM Kd indicates a relatively higher binding affinity as compared to a 500 μM Kd).

A “kit” is any manufacture (e.g., a package or container) comprising at least one reagent, e.g., a probe or small molecule, for specifically detecting and/or affecting the expression of a marker encompassed by the present invention. The kit may be promoted, distributed, or sold as a unit for performing the methods encompassed by the present invention. The kit may comprise one or more reagents necessary to express a composition useful in the methods encompassed by the present invention. In some embodiments, the kit may further comprise a reference standard, e.g., a nucleic acid encoding a protein that does not affect or regulate signaling pathways controlling cell growth, division, migration, survival or apoptosis. One skilled in the art can envision many such control proteins, including, but not limited to, common molecular tags (e.g., green fluorescent protein and beta-galactosidase), proteins not classified in any of pathway encompassing cell growth, division, migration, survival or apoptosis by GeneOntology reference, or ubiquitous housekeeping proteins. Reagents in the kit may be provided in individual containers or as mixtures of two or more reagents in a single container. In addition, instructional materials which describe the use of the compositions within the kit may be included.

As used herein, the term “linked” refers to the association of two or more molecules. The linkage may be covalent or non-covalent. The linkage also may be genetic (i.e., recombinantly fused). Such linkages may be achieved using a wide variety of art recognized techniques, such as chemical conjugation and recombinant protein production.

A “linker,” in some embodiments, may refer to an amino acid sequence that connects two proteins, polypeptides, peptides, domains, regions, or motifs and may provide a spacer function compatible with interaction of the two sub-binding domains so that the resulting polypeptide retains a specific binding affinity (e.g., scTCR) to a target molecule or retains signaling activity (e.g., TCR complex). In some embodiments, a linker is comprised of about two to about 35 amino acids, for instance, or about four to about 20 amino acids or about eight to about 15 amino acids or about 15 to about 25 amino acids.

The term “MAGEA1” refers to a particular member of the melanoma antigen gene family clustered on human chromosome Xq28 (e.g., chromosome X: 153,179,284-153,183,880 forward strand. GRCh38: CM000685.2) that is also known as cancer/testis antigen 1.1 (CT1.1); melanoma-associated antigen 1; MAGE1; melanoma antigen family A, 1 (directs expression of antigen MZ2-E); cancer/testis antigen family 1, member 1; melanoma-associated antigen MZ2-E; melanoma antigen family A1; cancer/testis antigen 1.1; melanoma antigen MAGE-1; MAGE-1 antigen; antigen Z2-E, MGC9326; and MAGE1A (Mao et al. (2019) J. Hematol. Oncol. 12:106; Fanipakdel et al. (2019) J. Cell Physiol. 234:12080-12086; Gu et al. (2018) Thorac. Cancer 9:431-438; Mecklenburg et al. (2017) Clin. Cancer Res. 23:1213-1219; Wang et al. (2016) Biochem. Biophys. Res. Commun. 473:959-965; Kozakova et al. (2015) Cell Cycle 14:920-930; Cannuyer et al. (2013) PLoS One 8:e5874; Pereira et al. (2012) Oncol. Rep. 27:1843-1848; Ogata et al. (2011) Ann. Surg. Oncol. 18:1195-1203; Roch et al. (2010) Anticancer Res. 30:1617-1623; Dango et al. (2010) Lung Cancer 67:290-295; van der Bruggen et al. (1991) Science 254:1643-1647). MAGEA1 is a melanoma antigen recognized by cytolytic T lymphocytes and is believed to be involved in transcriptional regulation through interaction with SNW1 and recruiting histone deacetylase HDAC1, as well as in inhibiting notch intracellular domain (NICD) transactivation, embryonal development, association with some herediatyr disorders (e.g., dyskeratosis congenital), and/or tumor transformation or aspects of tumor progression (e.g., gastric carcinomas, hepatocellular carcinomas, etc.). MAGEA1 is not highly expressed in normal tissues, except for testis, and is expressed in tumors of various histological types, such as melanoma, head & neck cancer, lung cancer, cervical cancer, hepatocellular carcinoma, colorectal cancer, gastrointestinal cancer, colorectal cancer, gastrointestinal cancer, breast invasive carcinoma, and bladder urothelial carcinoma.

The term “MAGEA1” is intended to include fragments, variants (e.g., allelic variants), and derivatives thereof. Representative human MAGEA1 cDNA and human MAGEA1 protein sequences are well-known in the art and are publicly available from the National Center for Biotechnology Information (NCBI) (see, for example, ncbi.nlm.nih.gov/gene/4100). For example, human MAGEA1 (NP_004979.3) is encodable by the transcript (NM_004988.5). Nucleic acid and polypeptide sequences of MAGEA1 orthologs in organisms other than humans are well-known and include, for example, chimpanzee MAGEA1 (XM_529226.2 and XP_529226.2) and mouse MAGEA1 (Chromosome X: 155088686-155089793; Ensembl Mus musculus version 104.39 (GRCm39)). Representative sequences of MAGEA1 sequences are also presented below in Table 3.

Anti-MAGEA1 antibodies suitable for detecting MAGEA1 protein are well-known in the art and include, for example, antibodies AM32863PU, AM50138PU, AP06212PU, AP13128PU-TA312178, TA39275, TA339275, TA339276, and TA347677 (OriGene, Rockville, MD); antibodies orb167376 and orb11016 (Biorbyt, Cambridge, United Kingdom); antibodies A03570 and AO3570-1 (Boster Bio, Pleasanton, CA); antibodies E22-11B2-E9 and N1C3 (GeneTex, Irvince, CA); antibodies AFLGC-MAGEA1, MA5-37821, and MA1-91067 (Invitrogen, Waltham, MA); antibodies ABIN2782493 and ABIN2782494 (Antibodies-online, Limerick, PA); and antibodies MA454 and 6C1 (Santa Cruz Biotechnology, Dallas, TX). In addition, reagents are well-known for detecting MAGEA1 expression. Moreover, multiple siRNA, shRNA, CRISPR constructs for modulating MAGEA1 expression can be found in the commercial product lists of a variety of companies, such as open reading frame (ORF) clones MG212171, MR212171, MR212171L3, MR212171L3V, MR212171L4, MR212171L4V, RC202134, RC202134L3, RC202134L3V, RC202134L4, RC202134L4V, and RG202134 (OriGene, Rockville, MD), CRISPR knockouts GA102785, GA202555, KN202134, KN202134BN, KN202134LP, KN202134RB, KN402134, and KN509652 (OriGene, Rockville, MD), and RNA interference (RNAi) clones, such as siRNA and shRNA clones, including SR302776, TL311617, SR410578, TL311617V, TTL516288, TL516288V, TL704467, TL04467V, TR311617, TR516288, and TR704467 (OriGene, Rockville, MD). It is to be noted that the term can further be used to refer to any combination of features described herein regarding MAGEA1 molecules. For example, any combination of sequence composition, percentage identify, sequence length, domain structure, functional activity, etc. can be used to describe a MAGEA1 molecule encompassed by the present invention.

The term “MAGEA1 antigen” or “MAGEA1 peptide antigen” or “MAGEA1-containing peptide antigen” or “MAGEA1 epitope” or “MAGEA1 peptide epitope” or “MAGEA1 peptide” refers to a naturally or synthetically produced immunogenic portion of MAGEA1. In some embodiments, MAGEA1 antigen protein can range in length from about 7, 8, 9, 10, 11, 12, 13, 14, 15, 16, 17, 18, 19, 20, 21, 22, 23, 24, 25 amino acids, or any range in between, inclusive, such as 8-15 amino acids. In some embodiments, MAGEA1 antigen protein can form a complex with an MHC (e.g., HLA) molecule such that a binding protein of this disclosure that recognizes a MAGEA1 peptide:MHC (e.g., HLA) complex can bind (e.g., specifically and/or selectively) to such a complex. Representative MAGEA1 peptide antigen sequences are shown in Table 1.

The term “major histocompatibility complex” (MHC) refers to glycoproteins that deliver peptide antigens to a cell surface. MHC class I molecules are heterodimers having a membrane spanning a chain (with three a domains) and a non-covalently associated b2 microglobulin. MHC class II molecules are composed of two transmembrane glycoproteins, a and b, both of which span the membrane. Each chain has two domains. MHC class I molecules deliver peptides originating in the cytosol to the cell surface, where a peptide antigen-MHC (pMHC) complex is recognized by CD8⁺ T cells. MHC class II molecules deliver peptides originating in the vesicular system to the cell surface, where they are recognized by CD4⁺ T cells. Human MHC is referred to as human leukocyte antigen (HLA).

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “prognosis” includes a prediction of the probable course and outcome of a cancer or the likelihood of recovery from the disease. In some embodiments, the use of statistical algorithms provides a prognosis of a cancer in an individual. For example, the prognosis may be surgery, development of a clinical subtype of a cancer, development of one or more clinical factors, or recovery from the disease.

As used herein, “percent identity” between amino acid sequences is synonymous with “percent homology,” which can be determined using the algorithm of Karlin and Altschul (1990) Proc. Natl. Acad. Sci. USA 87:2264-2268, modified by Karlin and Altschul (1993) Proc. Natl. Acad. Sci. USA 90:5873-5877. The noted algorithm is incorporated into the NBLAST and XBLAST programs of Altschul et al. (1990) J. Mol. Biol. 215:403-410. BLAST nucleotide searches are performed with the NBLAST program, score=100, wordlength=12, to obtain nucleotide sequences homologous to a polynucleotide described herein. BLAST protein searches are performed with the XBLAST program, score=50, wordlength=3, to obtain amino acid sequences homologous to a reference polypeptide. To obtain gapped alignments for comparison purposes, Gapped BLAST is utilized as described in Altschul et al. (1997) Nuc. Acids Res. 25:3389-3402. When utilizing BLAST and Gapped BLAST programs, the default parameters of the respective programs (e.g., XBLAST and NBLAST) may be used.

The phrase “pharmaceutically-acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body.

The term “ratio” refers to a relationship between two numbers (e.g., scores, summations, and the like). Although, ratios may be expressed in a particular order (e.g., a to b or a:b), one of ordinary skill in the art will recognize that the underlying relationship between the numbers may be expressed in any order without losing the significance of the underlying relationship, although observation and correlation of trends based on the ratio may be reversed.

The term “recombinant host cell” (or simply “host cell”) refers to a cell that comprises a nucleic acid that is not naturally present in the cell, such as a cell into which a recombinant expression vector has been introduced. It should be understood that cells according to the present invention is intended to refer not only to the particular subject cell, but also encompasses progeny of such a cell. Because certain modifications may occur in succeeding generations due to either mutation or environmental influences, such progeny may not, in fact, be identical to the parent cell, but are still included within the scope of the term cell according to the present invention.

The term “cancer response,” “response to immunotherapy,” or “response to modulators of T-cell mediated cytotoxicity/immunotherapy combination therapy” relates to any response of the hyperproliferative disorder (e.g., cancer) to a cancer agent, such as a modulator of T-cell mediated cytotoxicity, and an immunotherapy, preferably to a change in tumor mass and/or volume after initiation of neoadjuvant or adjuvant therapy. The term “neoadjuvant therapy” refers to a treatment given before the primary treatment. Examples of neoadjuvant therapy may include chemotherapy, radiation therapy, and hormone therapy. Hyperproliferative disorder response may be assessed, for example for efficacy or in a neoadjuvant or adjuvant situation, where the size of a tumor after systemic intervention may be compared to the initial size and dimensions as measured by CT, PET, mammogram, ultrasound or palpation. Responses may also be assessed by caliper measurement or pathological examination of the tumor after biopsy or surgical resection. Response may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response may be done early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy. In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more. Additional criteria for evaluating the response to cancer therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment may be expanded to include response to chemotherapy, probability of survival, probability of metastasis within a given time period, and probability of tumor recurrence. For example, in order to determine appropriate threshold values, a particular cancer therapeutic regimen may be administered to a population of subjects and the outcome may be correlated to biomarker measurements that were determined prior to administration of any cancer therapy. The outcome measurement may be pathologic response to therapy given in the neoadjuvant setting. Alternatively, outcome measures, such as overall survival and disease-free survival may be monitored over a period of time for subjects following cancer therapy for which biomarker measurement values are known. In certain embodiments, the doses administered are standard doses known in the art for cancer therapeutic agents. The period of time for which subjects are monitored may vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy may be determined using well-known methods in the art, such as those described in the Examples section.

As indicated, the terms may also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

The term “resistance” refers to an acquired or natural resistance of a cancer sample or a mammal to a cancer therapy (i.e., being nonresponsive to or having reduced or limited response to the therapeutic treatment), such as having a reduced response to a therapeutic treatment by 5%, 10%, 15%, 20%, 25%, 30%, 35%, 40%, 45%, 50%, 55%, 60%, 65%, 70%, 75%, 80%, 85%, 90%, 95%, 100%, or more, such 2-fold, 3-fold, 4-fold, 5-fold, 10-fold, 15-fold, 20-fold or more, or any range in between, inclusive. The reduction in response may be measured by comparing with the same cancer sample or mammal before the resistance is acquired, or by comparing with a different cancer sample or a mammal that is known to have no resistance to the therapeutic treatment. A typical acquired resistance to chemotherapy is called “multidrug resistance.” The multidrug resistance may be mediated by P-glycoprotein or may be mediated by other mechanisms, or it may occur when a mammal is infected with a multi-drug-resistant microorganism or a combination of microorganisms. The determination of resistance to a therapeutic treatment is routine in the art and within the skill of an ordinarily skilled clinician, for example, may be measured by cell proliferative assays and cell death assays as described herein as “sensitizing.” In some embodiments, the term “reverses resistance” means that the use of a second agent in combination with a primary cancer therapy (e.g., chemotherapeutic or radiation therapy) is able to produce a significant decrease in tumor volume at a level of statistical significance (e.g., p<0.05) when compared to tumor volume of untreated tumor in the circumstance where the primary cancer therapy (e.g., chemotherapeutic or radiation therapy) alone is unable to produce a statistically significant decrease in tumor volume compared to tumor volume of untreated tumor. This generally applies to tumor volume measurements made at a time when the untreated tumor is growing logarithmically.

The term “sample” used for detecting or determining the absence, presence, or level of at least one biomarker is typically brain tissue, cerebrospinal fluid, whole blood, plasma, serum, saliva, urine, stool (e.g., feces), tears, and any other bodily fluid (e.g., as described above under the definition of “body fluids”), or a tissue sample (e.g., biopsy) such as a skin, colon sample, or surgical resection tissue. In some embodiments, methods encompassed by the present invention further comprises obtaining the sample from the individual prior to detecting or determining the absence, presence, or level of at least one marker in the sample.

The term “sensitize” means to alter cancer cells or tumor cells in a way that allows for more effective treatment of the associated cancer with a cancer therapy (e.g., anti-immune checkpoint, chemotherapeutic, and/or radiation therapy). In some embodiments, normal cells are not affected to an extent that causes the normal cells to be unduly injured by the therapies. An increased sensitivity or a reduced sensitivity to a therapeutic treatment is measured according to a known method in the art for the particular treatment and methods described herein below, including, but not limited to, cell proliferative assays (Tanigawa et al. (1982) Cancer Res. 42:2159-2164) and cell death assays (Weisenthal et al. (1984) Cancer Res. 94:161-173; Weisenthal et al. (1985) Cancer Treat Rep. 69:615-632; Weisenthal et al., In: Kaspers G J L, Pieters R, Twentyman P R, Weisenthal L M, Veerman A J P, eds. Drug Resistance in Leukemia and Lymphoma. Langhorne, P A: Harwood Academic Publishers, 1993:415-432; Weisenthal (1994) Contrib. Gynecol. Obstet. 19:82-90). The sensitivity or resistance may also be measured in animal by measuring the tumor size reduction over a period of time, for example, 6 month for human and 4-6 weeks for mouse. A composition or a method sensitizes response to a therapeutic treatment if the increase in treatment sensitivity or the reduction in resistance is 5%, 10%, 15%, 20%, 25%, 30%, 35%, 40%, 45%, 50%, 55%, 60%, 65%, 70%, 75%, 80%, 85%, 90%, 95%, 100%, or more, such 2-fold, 3-fold, 4-fold, 5-fold, 10-fold, 15-fold, 20-fold or more, or any range in between, inclusive, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of a cancer therapy may be equally applied to methods for sensitizing hyperproliferative or otherwise cancerous cells (e.g., resistant cells) to the cancer therapy.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which may be screened for activity include, but are not limited to, peptides, peptidomimetics, nucleic acids, carbohydrates, small organic molecules (e.g., polyketides) (Cane et al. (1998) Science 282:63-68), and natural product extract libraries. In another embodiment, the compounds are small, organic non-peptidic compounds. In a further embodiment, a small molecule is not biosynthetic.

The term “specific binding” refers to binding protein binding to a predetermined antigen. Typically, the binding protein binds with an affinity (K_{D}) of approximately less than or equal to about 5×10⁻⁴ M, less than or equal to about 1×10⁻⁴ M, less than or equal to about 5×10⁻⁵ M, less than or equal to about 1×10⁻⁵ M, less than or equal to about 5×10⁻⁶ M, less than or equal to about 1×10⁻⁶ M, less than or equal to about 5×10⁻⁷ M, less than or equal to about 1×10⁻⁷ M, less than or equal to about 5×10⁻⁸ M, less than or equal to about 1×10⁻⁸ M, less than or equal to about 5×10⁻⁹ M, less than or equal to about 1×10⁻⁹ M, less than or equal to about 5×10⁻¹⁰ M, less than or equal to about 1×10⁻¹⁰ M, less than or equal to about 5×10⁻¹¹ M, less than or equal to about 1×10⁻¹¹ M, less than or equal to about 5×10⁻¹² M, less than or equal to about 1×10⁻¹² M, or even lower, or any range in between, inclusive, such as between about 1-50 micromolar, 1-100 micromolar, 0.1-500 micromolar, and the like, when determined by a binding assay, such as surface plasmon resonance (SPR) technology in a BIAcore™ assay instrument using an antigen of interest as the analyte and the binding protein as the ligand. In some embodiments, the binding protein binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. The phrases “a binding protein recognizing an antigen” and “a binding protein specific for an antigen” are used interchangeably herein with the term “a binding protein which binds specifically to an antigen.” Selective binding is a relative term referring to the ability of a binding protein to discriminate the binding of one antigen over another, such as a particular family member or antigen target over a related family member or antigen target. For example, analytical data provided in the Examples section demonstrates that binding proteins described herein specifically bind MAGEA1 immunogenic epitopes and/or selectively bind a number of related epitopes (e.g., MAGEA1 immunogenic epitopes and closely related sequences) discriminating such targets from the vast majority of other possible epitopes available in the human genome.

The term “subject” refers to any healthy animal, mammal or human, or any animal, mammal or human afflicted with a disorder characterized by MAGEA1 expression, such as a non-malignant disorder, a hyperproliferative disorder, or a relapse of a hyperproliferative disorder characterized by MAGEA1 expression. The term “subject” is interchangeable with “patient.”

The term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment may be expanded to include response to chemotherapy, probability of survival, probability of metastasis within a given time period, and probability of tumor recurrence.

The term “synergistic effect” refers to the combined effect of two or more agents (e.g., a MAGEA1-related agent described herein and another therapy, such as an additional MAGEA1-targeted TCR, anti-cancer therapy, immunotherapy, etc. for treating a disorder characterized by MAGEA1 expression) that is greater than the sum of the separate effects of the cancer agents/therapies alone.

As used herein, the term “T cell-mediated response” refers to a response mediated by T cells, including effector T cells (e.g., CD8⁺ cells) and helper T cells (e.g., CD4⁺ cells). T cell mediated responses include, for example, T cell cytotoxicity and proliferation.

A “transcribed polynucleotide” or “nucleotide transcript” is a polynucleotide (e.g., an mRNA, hnRNA, a cDNA, or an analog of such RNA or cDNA) which is complementary to or homologous with all or a portion of a mature mRNA made by transcription of a biomarker nucleic acid and normal post-transcriptional processing (e.g., splicing), if any, of the RNA transcript, and reverse transcription of the RNA transcript.

A “T cell” is an immune system cell that matures in the thymus and produces T cell receptors (TCRs). T cells may be naive (not exposed to antigen; increased expression of CD62L, CCR7, CD28, CD3, CD 127, and CD45RA, and decreased expression of CD45RO as compared to T_{CM}), memory T cells (T_{M}) (antigen-experienced and long-lived), and effector cells (antigen-experienced, cytotoxic). T_{M} may be further divided into subsets of central memory T cells (T_{CM}, increased expression of CD62L, CCR7, CD28, CD127, CD45RO, and CD95, and decreased expression of CD54RA as compared to naive T cells) and effector memory T cells (T_{EM}, decreased expression of CD62L, CCR7, CD28, CD45RA, and increased expression of CD127 as compared to naive T cells or T_{CM}). Effector T cells (T_{E}) refers to antigen-experienced CD8+ cytotoxic T lymphocytes that have decreased expression of CD62L, CCR7, CD28, and are positive for granzyme and perforin as compared to T_{CM}. Other exemplary T cells include regulatory T cells, such as CD4⁺ CD25⁺ (Foxp3⁺) regulatory T cells and Treg17 cells, as well as Trl, Th3, CD8⁺CD28, and Qa-1 restricted T cells.

Conventional T cells, also known as Tconv or Teffs, have effector functions (e.g., cytokine secretion, cytotoxic activity, anti-self-recognition, and the like) to increase immune responses by virtue of their expression of one or more T cell receptors. Tcons or Teffs are generally defined as any T cell population that is not a Treg and include, for example, naïve T cells, activated T cells, memory T cells, resting Tcons, or Tcons that have differentiated toward, for example, the Th1 or Th2 lineages. In some embodiments, Teffs are a subset of non-Treg T cells. In some embodiments, Teffs are CD4+ Teffs or CD8+ Teffs, such as CD4+ helper T lymphocytes (e.g., Th0, Th1, Tfh, or Th17) and CD8+ cytotoxic T lymphocytes. As described further herein, cytotoxic T cells are CD8+T lymphocytes. “Naïve Tcons” are CD4+ T cells that have differentiated in bone marrow, and successfully underwent a positive and negative processes of central selection in a thymus, but have not yet been activated by exposure to an antigen. Naïve Tcons are commonly characterized by surface expression of L-selectin (CD62L), absence of activation markers such as CD25, CD44 or CD69, and absence of memory markers such as CD45RO. Naïve Tcons are therefore believed to be quiescent and non-dividing, requiring interleukin-7 (IL-7) and interleukin-15 (IL-15) for homeostatic survival (see, at least WO 2010/101870). The presence and activity of such cells are undesired in the context of suppressing immune responses. Unlike Tregs, Tcons are not anergic and can proliferate in response to antigen-based T cell receptor activation (Lechler et al. (2001) Philos. Trans. R. Soc. Lond. Biol. Sci. 356:625-637).

“T effector” (“T_{eff}” or “T_{E}”) cells refers to T cells (e.g., CD4+ and CD8+ T cells) with cytolytic activities as well as T helper (Th) cells, which secrete cytokines and activate and direct other immune cells, but does not include regulatory T cells (Treg cells).

“T cell receptor” or “TCR” refers to an immunoglobulin superfamily member (having a variable binding domain, a constant domain, a transmembrane region, and a short cytoplasmic tail; see, e.g., Janeway et al. (1997) Curr. Biol. Publ. 4:33) that is capable of binding (e.g., e.g., specifically and/or selectively) to an antigen peptide bound to an MHC receptor. A TCR can be found on the surface of a cell or in soluble form and generally is comprised of a heterodimer having alpha and beta chains (also known as TCRα and TCRβ, respectively), or γ and δ chains (also known as TCRγ and TCRδ, respectively). Like immunoglobulins (e.g., antibodies), the extracellular portion of TCR chains (e.g., α-chain and β-chain) contain two immunoglobulin domains: a variable domain (e.g., α-chain variable domain or V_{α} and β-chain variable domain or V_{β}; typically amino acids 1 to 116 based on Kabat numbering (Kabat et al. (1991) “Sequences of Proteins of Immunological Interest, US Dept. Health and Human Services, Public Health Service National Institutes of Health, 5^{th} ed.) at the N-terminal end, and one constant domain (e.g., α-chain constant domain or C_{α}, typically amino acids 117 to 259 based on Kabat, β-chain constant domain or C_{β}, typically amino acids 117 to 295 based on Kabat) at the C-terminal end and adjacent to the cell membrane. Also like immunoglobulins, the variable domains contain complementary determining regions (“CDRs”, also called hypervariable regions or “HVRs”) separated by framework regions (“FRs”) (see, e.g., Fores et al. (1990) Proc. Natl. Acad Sci. US.A. 87:9138; Chothia et al. (1988) EMBO J. 7:3745; Lefranc et al. (2003) Dev. Comp. Immunol. 27:55). In some embodiments, a TCR is found on the surface of a T cell (or T lymphocyte) and associates with the CD3 complex. The source of a TCR encompassed by the present invention may be from various animal species, such as a human, mouse, rat, rabbit or other mammal.

The term “T cell receptor” or “TCR” should be understood to encompass full TCRs as well as antigen-binding portions or antigen-binding fragments thereof. In some embodiments, the TCR is an intact or full-length TCR, including TCRs in the aβ form or yo form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR may contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions (CDRs) involved in recognition of the peptide, MHC and/or MHC-peptide complex.

Nomenclature established by the International Immunogenetics Information System (IMGT) (see also Scaviner and Lefranc (2000) Exp. Clin. Immunogenet. 17:83-96 and 97-106; Folch and Lefranc (2000) Exp. Clin. Immunogenet, 17:107-114; T Cell Receptor Factsbook”, (2001) LeFranc and LeFranc, Academic Press, ISBN 0-12-441352-8). The IMGT provides unique sequences used to describe a TCR, and sequences described herein may be identified by reference to such unique sequences provided herein. TCR sequences are publicly available at the IMGT database at imgt.org.

As described above, native alpha/beta heterodimeric TCRs have an alpha chain and a beta chain. Broadly, each chain comprises variable, joining and constant regions, and the beta chain also usually contains a short diversity region between the variable and joining regions, but this diversity region is often considered as part of the joining region. Each variable region comprises three hypervariable CDRs (Complementarity Determining Regions) embedded in a framework sequence. CDR3 is well-known to be the main mediator of antigen recognition. There are several types of alpha chain variable (Vα) regions and several types of beta chain variable (Vβ) regions distinguished by their framework, CDR1 and CDR2 sequences, and by a partly defined CDR3 sequence. The Vα types are referred to in IMGT nomenclature by a unique TRAV number. For example, “TRAV4” defines a TCR Vα region having unique framework and CDR1 and CDR2 sequences, and a CDR3 sequence which is partly defined by an amino acid sequence which is preserved from TCR to TCR but which also includes an amino acid sequence which varies from TCR to TCR. Similarly, “TRBV2” defines a TCR Vβ region having unique framework and CDR1 and CDR2 sequences, but with only a partly defined CDR3 sequence. It is known that there are 54 alpha variable genes, of which 44 are functional, and 67 beta variable genes, of which 42 are functional, within the alpha and beta loci, respectively.

The joining regions of the TCR are similarly defined by the unique IMGT TRAJ and TRBJ nomenclature, and the constant regions by the IMGT TRAC and TRBC nomenclature. The beta chain diversity region is referred to in IMGT nomenclature by the abbreviation TRBD, and, as mentioned, the concatenated TRBD/TRBJ regions are often considered together as the joining region.

The gene pools that encode the TCR alpha and beta chains are located on different chromosomes and contain separate V, (D), J and C gene segments, which are brought together by rearrangement during T cell development. This leads to a very high diversity of T cell alpha and beta chains due to the large number of potential recombination events that occur between the 54 TCR alpha variable genes and 61 alpha J genes or between the 67 beta variable genes, two beta D genes and 13 beta J genes. The recombination process is not precise and introduces further diversity within the CDR3 region. Each alpha and beta variable gene may also comprise allelic variants, designated in IMGT nomenclature as TRAVxx*01 and *02, or TRBVx-x*01 and *02 respectively, thus further increasing the amount of variation. In the same way, some of the TRBJ sequences have two known variations. (Note that the absence of a “*” qualifier means that only one allele is known for the relevant sequence). The natural repertoire of human TCRs resulting from recombination and thymic selection has been estimated to comprise approximately 10⁶ unique beta chain sequences, determined from CDR3 diversity (Arstila et al. (1999) Science 286:958-961) and could be even higher (Robins et al. (2009) Blood 114:4099-4107). Each beta chain is estimated to pair with at least 25 different alpha chains, thus generating further diversity (Arstila et al. (1999) Science 286:958-961).

The term “TCR alpha variable domain” therefore refers to the concatenation of TRAV and TRAJ regions; a TRAV region only; or TRAV and a partial TRAJ region, and the term TCR alpha constant domain refers to the extracellular TRAC region, or to a C-terminal truncated or full length TRAC sequence. Likewise the term “TCR beta variable domain” refers to the concatenation of TRBV and TRBD/TRBJ regions; to the TRBV and TRBD regions only; to the TRBV and TRBJ regions only; or to the TRBV and partial TRBD and/or TRBJ regions, and the term TCR beta constant domain refers to the extracellular TRBC region, or to a C-terminal truncated or full length TRBC sequence. These TCR alpha variable domain and TCR beta variable domain nomenclature similarly applies to the variable domains of TCR gamma and TCR delta chains, respectively, for gamma/delta TCRs. An ordinarily skilled artisan can obtain TRAV, TRAJ, TRAC, TRBV, TRBJ, and TRBC gene sequences, such as through the publicly available IMGT database.

The term “TCR complex” refers to a complex formed by the association of CD3 with TCR. For example, a TCR complex may be composed of a CD3γ chain, a CD3δ chain, two CD3ε chains, a homodimer of CD3ζ chains, a TCRα chain, and a TCRβ chain. Alternatively, a TCR complex may be composed of a CD3γ chain, a CD3δ chain, two CD3ε chains, a homodimer of CD3ζ chains, a TCRγ chain, and a TCRδ chain.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human.

The terms “therapeutically effective amount” and “effective amount” means that amount of a substance that produces some desired effect, such as a desired local or systemic therapeutic effect, in at least a sub-population of cells in an animal at a reasonable benefit/risk ratio applicable to any treatment. In some embodiments, a therapeutically effective amount of a substance will depend on the substance's therapeutic index, solubility, pharmacokinetics, half-life, and the like. Toxicity and therapeutic efficacy of subject compounds may be determined by standard pharmaceutical procedures in cell cultures or experimental animals, e.g., for determining the LD₅₀ and the ED₅₀. In some embodiments, compositions that exhibit large therapeutic indices are used. In some embodiments, the LD₅₀ (lethal dosage) may be measured and may be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) may be measured and may be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. Also, similarly, the IC₅₀ may be measured and may be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. In some embodiments, T cell immune response in an assay may be increased by at least about 10%, 15%, 20%, 25%, 30%, 35%, 40%, 45%, 50%, 55%, 60%, 65%, 70%, 75%, 80%, 85%, 90%, 95%, or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in a viral load may be achieved.

The term “treat” refers to the therapeutic management or improvement of a condition (e.g., a disease or disorder) of interest. Treatment may include, but is not limited to, administering an agent or composition (e.g., a pharmaceutical composition) to a subject. Treatment is typically undertaken in an effort to alter the course of a disease (which term is used to indicate any disease, disorder, syndrome or undesirable condition warranting or potentially warranting therapy) in a manner beneficial to the subject. The effect of treatment may include reversing, alleviating, reducing severity of, delaying the onset of, curing, inhibiting the progression of, and/or reducing the likelihood of occurrence or recurrence of the disease or one or more symptoms or manifestations of the disease. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. A therapeutic agent may be administered to a subject who has a disease or is at increased risk of developing a disease relative to a member of the general population. In some embodiments, a therapeutic agent may be administered to a subject who has had a disease but no longer shows evidence of the disease. The agent may be administered e.g., to reduce the likelihood of recurrence of evident disease. A therapeutic agent may be administered prophylactically, i.e., before development of any symptom or manifestation of a disease. “Prophylactic treatment” refers to providing medical and/or surgical management to a subject who has not developed a disease or does not show evidence of a disease in order, e.g., to reduce the likelihood that the disease will occur or to reduce the severity of the disease should it occur. The subject may have been identified as being at risk of developing the disease (e.g., at increased risk relative to the general population or as having a risk factor that increases the likelihood of developing the disease.

The term “unresponsiveness” includes refractivity of cancer cells to therapy or refractivity of therapeutic cells, such as immune cells, to stimulation, e.g., stimulation via an activating receptor or a cytokine. Unresponsiveness may occur, e.g., because of exposure to immunosuppressants or exposure to high doses of antigen. As used herein, the term “anergy” or “tolerance” includes refractivity to activating receptor-mediated stimulation. Such refractivity is generally antigen-specific and persists after exposure to the tolerizing antigen has ceased. For example, anergy in T cells (as opposed to unresponsiveness) is characterized by lack of cytokine production, e.g., IL-2. T cell anergy occurs when T cells are exposed to antigen and receive a first signal (a T cell receptor or CD-3 mediated signal) in the absence of a second signal (a costimulatory signal). Under these conditions, reexposure of the cells to the same antigen (even if reexposure occurs in the presence of a costimulatory polypeptide) results in failure to produce cytokines and, thus, failure to proliferate. Anergic T cells may, however, proliferate if cultured with cytokines (e.g., IL-2). For example, T cell anergy may also be observed by the lack of IL-2 production by T lymphocytes as measured by ELISA or by a proliferation assay using an indicator cell line. Alternatively, a reporter gene construct may be used. For example, anergic T cells fail to initiate IL-2 gene transcription induced by a heterologous promoter under the control of the 5′ IL-2 gene enhancer or by a multimer of the AP1 sequence that may be found within the enhancer (Kang et al. (1992) Science 257:1134).

The term “vaccine” refers to a pharmaceutical composition that elicits an immune response to an antigen of interest. The vaccine may also confer protective immunity upon a subject.

The term “variable region” or “variable domain” refers to the domain of an immunoglobulin superfamily binding protein (e.g., a TCR α-chain or β-chain (or γ chain and δ chain for γδ TCRs)) that is involved in binding of the immunoglobulin superfamily binding protein (e.g., TCR) to antigen. The variable domains of the α-chain and β-chain (V_{α} and V_{β}, respectively) of a native TCR generally have similar structures, with each domain comprising four conserved framework regions (FRs) and three CDRs. The V_{α} domain is encoded by two separate DNA segments, the variable gene segment and the joining gene segment (V-J); the V_{β} domain is encoded by three separate DNA segments, the variable gene segment, the diversity gene segment, and the joining gene segment (V-D-J). A single V_{α} or V domain may be sufficient to confer antigen-binding specificity. Furthermore, TCRs that bind a particular antigen may be isolated using a V_{α} or V_{β} domain from a TCR that binds the antigen to screen a library of complementary V_{α} or V_{β} domains, respectively.

The term “vector” refers to a nucleic acid molecule capable of transporting another nucleic acid to which it has been linked. In some embodiments, a vector is an episome, i.e., a nucleic acid capable of extra-chromosomal replication. In some embodiments, vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops, which, in their vector form are not bound to the chromosome. In the present specification, “plasmid” and “vector” are used interchangeably as the plasmid is the most commonly used form of vector. However, as will be appreciated by those skilled in the art, the present invention is intended to include such other forms of expression vectors that serve equivalent functions and which become subsequently known in the art.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA encoding a biomarker nucleic acid (or any portion thereof) may be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

### II. Peptides

In certain aspects, provided herein are methods and compositions for the treatment and/or prevention of disorders associated with MAGEA1 expression through the induction of an immune response against MAGEA1 or cells expressing MAGEA1 relating to administration of MAGEA1 immunogenic peptides, nucleic acids encoding same, and/or cells expressing same, described herein.

In certain embodiments, the MAGEA1 immunogenic peptide comprises (e.g., consists of) a peptide epitope selected from peptide sequences listed in Table 1, such as Table 1A. Peptide epitopes described herein may be combined with MHC molecules, such as particular HLA molecules having particular HLA alpha chain alleles. For example, Table 1A peptides were identified in association with an MHC whose alpha chain had an HLA-A*02 serotype, such as that encoded by an HLA-A*02:01 allele, as described further in the Examples section. In some embodiments, MAGEA1 immunogenic peptides may be combined with an MHC molecule, wherein the MHC molecule comprises an MHC alpha chain that is an HLA serotype selected from the group consisting of HLA-A*02, HLA-A*03, HLA-A*01, HLA-A*11, HLA-A*24, HLA-B*07, HLA-C*07, HLA-C*01, HLA-C*02, HLA-C*03, HLA-C*04, HLA-C*05, HLA-C*06, HLA-C*08, HLA-C*12, HLA-C*14, HLA-C*15, HLA-C*16, HLA-C*17, and HLA-C*18, optionally wherein the HLA allele is selected from the group consisting of HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:12, HLA-A*02:13, HLA-A*02:14, HLA-A*02:16, HLA-A*02:17, HLA-A*02:19, HLA-A*02:20, HLA-A*02:22, HLA-A*02:24, HLA-A*02:30, HLA-A*02:42, HLA-A*02:53, HLA-A*02:60, HLA-A*02:74 allele, HLA-A*03:01, HLA-A*03:02, HLA-A*03:05, HLA-A*03:07, HLA-A*01:01, HLA-A*01:02, HLA-A*01:03, HLA-A*01:16 allele, HLA-A*11:01, HLA-A*11:02, HLA-A*11:03, HLA-A*11:04, HLA-A*11:05, HLA-A*11:19 allele, HLA-A*24:02, HLA-A*24:03, HLA-A*24:05, HLA-A*24:07, HLA-A*24:08, HLA-A*24:10, HLA-A*24:14, HLA-A*24:17, HLA-A*24:20, HLA-A*24:22, HLA-A*24:25, HLA-A*24:26, HLA-A*24:58 allele, HLA-B*07:02, HLA-B*07:04, HLA-B*07:05, HLA-B*07:09, HLA-B*07:10, HLA-B*07:15, HLA-B*07:21, HLA-C*07:02, HLA-C*07:01, HLA-C*04:01, HLA-C*06:02, HLA-C*03:04, HLA-C*05:01, HLA-C*16:01, HLA-C*02:02, HLA-C*03:03, HLA-C*12:03, HLA-C*08:02, HLA-C*01:02, HLA-C*17:01, HLA-C*15:02, HLA-C*14:02, HLA-C*12:02, HLA-C*07:04, HLA-C*08:01, HLA-C*03:02, HLA-C*18:01, HLA-C*15:05, HLA-C*16:02, HLA-C*08:04, HLA-C*03:05, and HLA-C*14:03 allele. In some embodiments, the MAGEA1 immunogenic peptides are derived from a human MAGEA1 protein and/or a MAGEA1 protein shown in Table 3. In some embodiments, one or more MAGEA1 immunogenic peptides are administered alone or in combination with an adjuvant.

In certain aspects, provided herein are compositions comprising one or more MAGEA1 immunogenic peptides described herein and an adjuvant.

TABLE 1
MAGEA1 epitopes
Table 1A
MAGEA1 epitopes presented by HLA serotype HLA-A*02

Peptide Epitopes
KVLEYVIKV (SEQ ID NO: 1)
VLEYVIKV (SEQ ID NO: 2)
KVLEYVIK (SEQ ID NO: 3)
*Included in Table 1, such as Table 1A are peptide epitopes, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any sequence listed in Table 1, such as Table 1A, or a portion thereof. Such polypeptides may have a function of the full-length peptide or polypeptide as described further herein.

In some embodiments, provided herein are MAGEA1 polypeptides and/or nucleic acids encoding MAGEA1 polypeptides. In some embodiments, MAGEA1 polypeptides are polypeptides that include an amino acid sequence of sufficient length to elicit a MAGEA1-specific immune response. In certain embodiments, the MAGEA1 polypeptide also includes amino acids that do not correspond to the amino acid sequence (e.g., a fusion protein comprising a MAGEA1 amino acid sequence and an amino acid sequence corresponding to a non-MAGEA1 protein or polypeptide). In some embodiments, the MAGEA1 polypeptide only includes amino acid sequence corresponding to a MAGEA1 protein or fragment thereof.

In some embodiments, the MAGEA1 polypeptide has an amino acid sequence that comprises, consists essentially of, or consists of at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 373, or more, or any range in between inclusive (e.g., 7-25, 8-22, 9-22, etc.) consecutive amino acids of a MAGEA1 protein amino acid sequence, such as those set forth in Table 3. In some embodiments, the consecutive amino acids are identical to an amino acid sequence of MAGEA1 set forth in Table 3. In some embodiments, MAGEA1 polypeptides comprise, consist essentially of, or consist of one or more peptide epitopes selected from the group consisting of MAGEA1 peptide epitopes listed in Table 1, such as Table 1A.

As is well-known to those skilled in the art, polypeptides having substantial sequence similarities can cause identical or very similar immune reaction in a host animal. Accordingly, in some embodiments, a derivative, equivalent, variant, fragment, or mutant of a MAGEA1 immunogenic peptide described herein or fragment thereof may also suitable for the methods and compositions provided herein.

In some embodiments, variations or derivatives of the MAGEA1 immunogenic polypeptides are provided herein. The altered polypeptide may have an altered amino acid sequence, for example by conservative substitution, yet still elicits immune responses which react with the unaltered protein antigen, and are considered functional equivalents. As used herein, the term “conservative substitution” denotes the replacement of an amino acid residue by another, biologically similar residue. It is well-known in the art that the amino acids within the same conservative group may typically substitute for one another without substantially affecting the function of a protein. According to certain embodiments, the derivative, equivalents, variants, or mutants of the ligand-binding domain of a MAGEA1 immunogenic peptide are polypeptides that are at least 85% homologous to the sequence of a MAGEA1 immunogenic peptide described herein or fragment thereof. In some embodiments, the homology is at least 90%, at least 95%, at least 98%, or more.

Immunogenic peptides encompassed by the present invention may comprise a peptide epitope derived from a MAGEA1 protein, such as those listed in Table 1, such as Table 1A. In some embodiments, the immunogenic peptide is 8, 9, 10, 11, 12, 13, 14, or 15 amino acids in length. In some embodiments, the peptide amino acid sequences is modified, which may include conservative or non-conservative mutations. A peptide may comprise at most 1, 2, 3, 4, or more mutations. In some embodiments, a peptide may comprise at least 1, 2, 3, 4, or more mutations.

In some embodiments, a peptide may be chemically modified. For example, a peptide can be mutated to modify peptide properties such as detectability, stability, biodistribution, pharmacokinetics, half-life, surface charge, hydrophobicity, conjugation sites, pH, function, and the like. N-methylation is one example of methylation that can occur in a peptide of the disclosure. In some embodiments, a peptide may be modified by methylation on free amines such as by reductive methylation with formaldehyde and sodium cyanoborohydride.

A chemical modification may comprise a polymer, a polyether, polyethylene glycol, a biopolymer, a zwitterionic polymer, a polyamino acid, a fatty acid, a dendrimer, an Fc region, a simple saturated carbon chain such as palmitate or myristolate, or albumin. The chemical modification of a peptide with an Fc region may be a fusion Fc-peptide. A polyamino acid may include, for example, a poly amino acid sequence with repeated single amino acids (e.g., poly glycine), and a poly amino acid sequence with mixed poly amino acid sequences that may or may not follow a pattern, or any combination of the foregoing. In some embodiments, the peptides encompassed by the present disclosure may be modified such that the modification increases the stability and/or the half-life of the peptides. In some embodiments, the attachment of a hydrophobic moiety, such as to the N-terminus, the C-terminus, or an internal amino acid, can be used to extend half-life of a peptide encompassed by the present disclosure. In other embodiments, a peptide may include post-translational modifications (e.g., methylation and/or amidation), which can affect, for example, serum half-life. In some embodiments, simple carbon chains (e.g., by myristoylation and/or palmitylation) can be conjugated to the fusion proteins or peptides. In some embodiments, the simple carbon chains may render the fusion proteins or peptides easily separable from the unconjugated material. For example, methods that may be used to separate the fusion proteins or peptides from the unconjugated material include, but are not limited to, solvent extraction and reverse phase chromatography. The lipophilic moieties can extend half-life through reversible binding to serum albumin. The conjugated moieties may be lipophilic moieties that extend half-life of the peptides through reversible binding to serum albumin. In some embodiments, the lipophilic moiety may be cholesterol or a cholesterol derivative, including cholestenes, cholestanes, cholestadienes and oxysterols. In some embodiments, the peptides may be conjugated to myristic acid (tetradecanoic acid) or a derivative thereof. In other embodiments, a peptide may be coupled (e.g., conjugated) to a half-life modifying agent. Examples of half-life modifying agents include but are not limited to: a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, or a molecule that binds to albumin. In some embodiments, a spacer or linker may be coupled to a peptide, such as 1, 2, 3, 4, or more amino acid residues that serve as a spacer or linker in order to facilitate conjugation or fusion to another molecule, as well as to facilitate cleavage of the peptide from such conjugated or fused molecules. In some embodiments, fusion proteins or peptides may be conjugated to other moieties that, for example, can modify or effect changes to the properties of the peptides.

A peptide may, in some embodiments, be covalently linked to a moiety. In some embodiments, the covalently linked moiety comprises an affinity tag or a label. The affinity tag may be selected from the group consisting of glutathione-S-transferase (GST), calmodulin binding protein (CBP), protein C tag, Myc tag, HaloTag, HA tag, Flag® tag, His tag, biotin tag, and V5 tag. The label may be a fluorescent protein. In some embodiments, the covalently linked moiety is selected from the group consisting of an inflammatory agent, an anti-inflammatory agent, a cytokine, a toxin, a cytotoxic molecule, a radioactive isotope, or an antibody such as a single-chain Fv.

A peptide may be conjugated to an agent used in imaging, research, therapeutics, theranostics, pharmaceuticals, chemotherapy, chelation therapy, targeted drug delivery, and radiotherapy. In some embodiments, a peptide may be conjugated to or fused with detectable agents, such as a fluorophore, a near-infrared dye, a contrast agent, a nanoparticle, a metal-containing nanoparticle, a metal chelate, an X-ray contrast agent, a PET agent, a metal, a radioisotope, a dye, radionuclide chelator, or another suitable material that can be used in imaging. In some embodiments, 1, 2, 3, 4, 5, 6, 7, 8, 9, 10, or more detectable moieties may be linked to a peptide. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212. In some embodiments, the near-infrared dyes are not easily quenched by biological tissues and fluids. In some embodiments, the fluorophore is a fluorescent agent emitting electromagnetic radiation at a wavelength between 650 nm and 4000 nm, such emissions being used to detect such agent. Non-limiting examples of fluorescent dyes that may be used as a conjugating molecule include DyLight®-680, DyLight®-750, VivoTag®-750, DyLight®-800, IRDye®-800, VivoTag®-680, Cy5.5, ZQ800, or indocyanine green (ICG). In some embodiments, near infrared dyes often include cyanine dyes (e.g., Cy7, Cy5.5, and Cy5). Additional non-limiting examples of fluorescent dyes for use as a conjugating molecule in the present disclosure include acradine orange or yellow, Alexa Fluors® (e.g., Alexa Fluor® 790, 750, 700, 680, 660, and 647) and any derivative thereof, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl) anthracene, 5,12-bis(phenylethynyl) naththacene, bisbenzimide, brainbow, calcein, carbodyfluorescein and any derivative thereof, 1-chloro-9,10-bis(phenylethynyl) anthracene and any derivative thereof, DAPI, DiOC6, DyLight Fluors and any derivative thereof, epicocconone, ethidium bromide, FLASH-EDT2, Fluo dye and any derivative thereof, FluoProbe and any derivative thereof, Fluorescein and any derivative thereof, Fura and any derivative thereof, GelGreen and any derivative thereof, GelRed and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR™ and any derivative thereof, synapto-pHluorin, tetraphenyl butadiene, tetrasodium tris, Texas Red, Titan Yellow, TSQ, umbelliferone, violanthrone, yellow fluroescent protein and YOYO-1. Other Suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green® Dyes (e.g., Oregon Green® 488, Oregon Green® 500, Oregon Green® 514, etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR® dyes (e.g., ALEXA FLUOR® 350, ALEXA FLUOR® 488, ALEXA FLUOR® 532, ALEXA FLUOR® 546, ALEXA FLUOR® 568, ALEXA FLUOR® 594, ALEXA FLUOR® 633, ALEXA FLUOR® 660, ALEXA FLUOR® 680, etc.), BODIPY® dyes (e.g., BODIPY® FL, BODIPY® R6G, BODIPY® TMR, BODIPY® TR, BODIPY® 530/550, BODIPY® 558/568, BODIPY® 564/570, BODIPY® 576/589, BODIPY® 581/591, BODIPY® 630/650, BODIPY® 650/665, etc.), IRDyes (e.g., IRD40, IRD 700, IRD 800, etc.), and the like. Additional suitable detectable agents are described in PCT/US14/56177. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212.

A peptide may be conjugated to a radiosensitizer or photosensitizer. Examples of radiosensitizers include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers include but are not limited to: fluorescent molecules or beads that generate heat when illuminated, nanoparticles, porphyrins and porphyrin derivatives (e.g., chlorins, bacteriochlorins, isobacteriochlorins, phthalocyanines, and naphthalocyanines), metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach allows for highly specific targeting of cells of interest (e.g., immune cells) using both a therapeutic agent (e.g., drug) and electromagnetic energy (e.g., radiation or light) concurrently. In some embodiments, the peptide is fused with, or covalently or non-covalently linked to the agent, for example, directly or via a linker.

In some embodiments, the binding protein may be chemically modified. For example, a binding protein may be mutated to modify peptide properties such as detectability, stability, biodistribution, pharmacokinetics, half-life, surface charge, hydrophobicity, conjugation sites, pH, function, and the like. N-methylation is one example of methylation that can occur in a binding protein encompassed by the present invention. In some embodiments, a binding protein may be modified by methylation on free amines such as by reductive methylation with formaldehyde and sodium cyanoborohydride.

A chemical modification may comprise a polymer, a polyether, polyethylene glycol, a biopolymer, a zwitterionic polymer, a polyamino acid, a fatty acid, a dendrimer, an Fc region, a simple saturated carbon chain such as palmitate or myristolate, or albumin. The chemical modification of a binding protein with an Fc region may be a fusion Fc-protein. A polyamino acid may include, for example, a poly amino acid sequence with repeated single amino acids (e.g., poly glycine), and a poly amino acid sequence with mixed poly amino acid sequences that may or may not follow a pattern, or any combination of the foregoing.

In some embodiments, the binding proteins encompassed by the present invention may be modified. In some embodiments, the modifications having substantial or significant sequence identity to a parent binding protein to generate a functional variant that maintains one or more biophysical and/or biological activities of the parent binding protein (e.g., maintain pMHC binding specificity). In some embodiments, the mutation is a conservative amino acid substitution.

In some embodiments, binding proteins encompassed by the present invention may comprise synthetic amino acids in place of one or more naturally-occurring amino acids. Such synthetic amino acids are well-known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, a-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, oc-aminocyclohexane carboxylic acid, α-aminocycloheptane carboxylic acid, a-(2-amino-2-norbornane)-carboxylic acid, α,γ-diaminobutyric acid, β-diaminopropionic acid, homophenylalanine, and oc-tert-butylglycine.

Binding proteins encompassed by the present invention may be glycosylated, amidated, carboxylated, phosphorylated, esterified, N-acylated, cyclized (e.g., via a disulfide bridge), or converted into an acid addition salt and/or optionally dimerized or polymerized, or conjugated.

In some embodiments, the attachment of a hydrophobic moiety, such as to the N-terminus, the C-terminus, or an internal amino acid, may be used to extend half-life of a peptide encompassed by the present invention. In other embodiments, a binding protein may include post-translational modifications (e.g., methylation and/or amidation), which can affect, for example, serum half-life. In some embodiments, simple carbon chains (e.g., by myristoylation and/or palmitylation) may be conjugated to the binding proteins. In some embodiments, the simple carbon chains may render the binding proteins easily separable from the unconjugated material. For example, methods that may be used to separate the binding proteins from the unconjugated material include, but are not limited to, solvent extraction and reverse phase chromatography. The lipophilic moieties can extend half-life through reversible binding to serum albumin. The conjugated moieties may be lipophilic moieties that extend half-life of the peptides through reversible binding to serum albumin. In some embodiments, the lipophilic moiety may be cholesterol or a cholesterol derivative, including cholestenes, cholestanes, cholestadienes and oxysterols. In some embodiments, the binding proteins may be conjugated to myristic acid (tetradecanoic acid) or a derivative thereof. In other embodiments, a binding protein may be coupled (e.g., conjugated) to a half-life modifying agent. Examples of half-life modifying agents include but are not limited to: a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, or a molecule that binds to albumin. In some embodiments, a spacer or linker may be coupled to a binding protein, such as 1, 2, 3, 4, or more amino acid residues that serve as a spacer or linker in order to facilitate conjugation or fusion to another molecule, as well as to facilitate cleavage of the peptide from such conjugated or fused molecules. In some embodiments, binding proteins may be conjugated to other moieties that, for example, can modify or effect changes to the properties of the binding proteins.

A protein such as a peptide may be produced recombinantly or synthetically, such as by solid-phase peptide synthesis or solution-phase peptide synthesis. Protein synthesis may be performed by known synthetic methods, such as using fluorenylmethyloxycarbonyl (Fmoc) chemistry or by butyloxycarbonyl (Boc) chemistry. Protein fragments may be joined together enzymatically or synthetically.

In an aspect encompassed by the present invention, provided herein are methods of producing a protein described herein, comprising the steps of: (i) culturing a transformed host cell which has been transformed by a nucleic acid comprising a sequence encoding a binding protein described herein under conditions suitable to allow expression of said binding protein; and (ii) recovering the expressed binding protein.

Methods useful for isolating and purifying recombinantly produced binding protein, by way of example, may include obtaining supernatants from suitable host cell/vector systems that secrete the binding protein into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of binding proteins described herein include batch cell culture, which is monitored and controlled to maintain appropriate culture conditions. Purification of the binding protein may be performed according to methods described herein and known in the art.

In some embodiments, provided herein is a nucleic acid encoding a MAGEA1 immunogenic peptide described herein or fragment thereof, such as a DNA molecule encoding a MAGEA1 immunogenic peptide. In some embodiments, the composition comprises an expression vector comprising an open reading frame encoding a MAGEA1 immunogenic peptide described herein or fragment thereof. In some embodiments, the nucleic acid includes regulatory elements necessary for expression of the open reading frame. Such elements may include, for example, a promoter, an initiation codon, a stop codon, and a polyadenylation signal. In addition, enhancers may be included. These elements may be operably linked to a sequence that encodes the MAGEA1 immunogenic polypeptide or fragment thereof. Representative vectors, promoters, regulatory elements, and the like useful for expressing proteins such as peptide are described further below.

### III. MHC-Peptide Complexes

In certain aspects, provided herein are compositions comprising a MAGEA1 immunogenic peptide described herein and a MHC molecule. In some embodiments, the MAGEA1 immunogenic peptide forms a stable complex with the MHC molecule.

MHC proteins may be conjugated to an agent, such as a detection moiety, readiosensitizer, photosensitizer, and the like, and/or may be chemically modified as described above regarding peptides.

The MHC proteins provided and used in the compositions and methods encompassed by the present disclosure may be any suitable MHC molecules known in the art. Generally, they have the formula (α-β-P)ₙ, where n is at least 2, for example between 2-10, e.g., 4. α is an α chain of a class I or class II MHC protein. β is a β chain, herein defined as the β chain of a class II MHC protein or β₂ microglobulin for a MHC class I protein. P is a peptide antigen.

In some embodiments, the MHC proteins are MHC class I complexes, such as HLA I complexes.

The MHC proteins may be from any mammalian or avian species, e.g., primate sp., particularly humans; rodents, including mice, rats and hamsters; rabbits; equines, bovines, canines, felines; etc. For instance, the MHC protein may be derived the human HLA proteins or the murine H-2 proteins. HLA proteins include the class II subunits HLA-DPα, HLA-DPβ, HLA-DQα, HLA-DQβ, HLA-DRα and HLA-DRβ, and the class I proteins HLA-A, HLA-B, HLA-C, and B2-microglobulin. H-2 proteins include the class I subunits H-2K, H-2D, H-2L, and the class II subunits I-Aα, I-Aβ, I-Eα and I-Eβ, and β2-microglobulin. Sequences of some representative MHC proteins may be found in Kabat et al. Sequences of Proteins of Immunological Interest, NIH Publication No. 91-3242, pp 724-815. MHC protein subunits suitable for use in the present invention are a soluble form of the normally membrane-bound protein, which is prepared as known in the art, for instance by deletion of the transmembrane domain and the cytoplasmic domain.

For class I proteins, the soluble form may include the α1, α2 and α3 domain. Soluble class II subunits may include the α1 and α2 domains for the a subunit, and the β1 and β2 domains for the β subunit.

The α and β subunits may be separately produced and allowed to associate in vitro to form a stable heteroduplex complex, or both of the subunits may be expressed in a single cell. Methods for producing MHC subunits are known in the art.

In certain embodiments, the MHC-peptide complex comprises a peptide epitope selected from Table 1 and an MHC. In some embodiments, the MHC molecule comprises an MHC alpha chain that is an HLA serotype selected from the group consisting of HLA-A*02, HLA-A*03, HLA-A*01, HLA-A*11, HLA-A*24, HLA-B*07, HLA-C*07, HLA-C*01, HLA-C*02, HLA-C*03, HLA-C*04, HLA-C*05, HLA-C*06, HLA-C*08, HLA-C*12, HLA-C*14, HLA-C*15, HLA-C*16, HLA-C*17, and HLA-C*18, optionally wherein the HLA allele is selected from the group consisting of HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:12, HLA-A*02:13, HLA-A*02:14, HLA-A*02:16, HLA-A*02:17, HLA-A*02:19, HLA-A*02:20, HLA-A*02:22, HLA-A*02:24, HLA-A*02:30, HLA-A*02:42, HLA-A*02:53, HLA-A*02:60, HLA-A*02:74 allele, HLA-A*03:01, HLA-A*03:02, HLA-A*03:05, HLA-A*03:07, HLA-A*01:01, HLA-A*01:02, HLA-A*01:03, HLA-A*01:16 allele, HLA-A*11:01, HLA-A*11:02, HLA-A*11:03, HLA-A*11:04, HLA-A*11:05, HLA-A*11:19 allele, HLA-A*24:02, HLA-A*24:03, HLA-A*24:05, HLA-A*24:07, HLA-A*24:08, HLA-A*24:10, HLA-A*24:14, HLA-A*24:17, HLA-A*24:20, HLA-A*24:22, HLA-A*24:25, HLA-A*24:26, HLA-A*24:58 allele, HLA-B*07:02, HLA-B*07:04, HLA-B*07:05, HLA-B*07:09, HLA-B*07:10, HLA-B*07:15, HLA-B*07:21, HLA-C*07:02, HLA-C*07:01, HLA-C*04:01, HLA-C*06:02, HLA-C*03:04, HLA-C*05:01, HLA-C*16:01, HLA-C*02:02, HLA-C*03:03, HLA-C*12:03, HLA-C*08:02, HLA-C*01:02, HLA-C*17:01, HLA-C*15:02, HLA-C*14:02, HLA-C*12:02, HLA-C*07:04, HLA-C*08:01, HLA-C*03:02, HLA-C*18:01, HLA-C*15:05, HLA-C*16:02, HLA-C*08:04, HLA-C*03:05, and HLA-C*14:03 allele. In some embodiments, the the MHC-peptide complex comprises a peptide epitope selected from Table 1A and an MHC whose alpha chain has an HLA-A*02 serotype, such as that encoded by an HLA-A*02:01 allele.

To prepare the MHC-peptide complex, the subunits may be combined with an antigenic peptide and allowed to fold in vitro to form a stable heterodimer complex with intrachain disulfide bonded domains. The peptide may be included in the initial folding reaction, or may be added to the empty heterodimer in a later step. In the compositions and methods encompassed by the present invention, this is a MAGEA1 immunogenic peptide or fragment thereof. Conditions that permit folding and association of the subunits and peptide are known in the art. As one example, roughly equimolar amounts of solubilized a and B subunits may be mixed in a solution of urea. Refolding is initiated by dilution or dialysis into a buffered solution without urea. Peptides may be loaded into empty class II heterodimers at about pH 5 to 5.5 for about 1 to 3 days, followed by neutralization, concentration and buffer exchange. However, the specific folding conditions are not critical for the practice of the invention.

The monomeric complex (α-β-P) (herein monomer) may be multimerized, for example, for a MHC tetramer. The resulting multimer is stable over long periods of time. Preferably, the multimer may be formed by binding the monomers to a multivalent entity through specific attachment sites on the α or β subunit, as known in the art (e.g., as described in U.S. Pat. No. 5,635,363). The MHC proteins, in either their monomeric or multimeric forms, may also be conjugated to beads or any other support.

The multimeric complex may be labeled, so as to be directly detectable when used in immunostaining or other methods known in the art, or may be used in conjunction with secondary labeled immunoreagents which specifically and/or selectively bind the complex (e.g., bind to a MHC protein subunit) as known in the art. For example, the detectable label may be a fluorophore, such as fluorescein isothiocyanate (FITC), rhodamine, Texas Red, phycoerythrin (PE), allophycocyanin (APC), Brilliant Violet™ 421, Brilliant UV™ 395, Brilliant Violet™ 480, Brilliant Violet™ 421 (BV421), Brilliant Blue™ 515, APC-R700, or APC-Fire750. In some embodiments, the multimeric complex is labeled by a moiety that is capable of specifically and/or selectively binding another moiety. For instance, the label may be biotin, streptavidin, an oligonucleotide, or a ligand. Other labels of interest may include fluorochromes, dyes, enzymes, chemiluminescers, particles, radioisotopes, or other directly or indirectly detectable agent.

In some embodiments, a cell presenting an immunogenic peptides in context of an MHC molecule on the cell surface is generated by transfecting or transducing the cell with a vector (e.g., a viral vector) that comprising nucleic acid that encodes a recombinant or heterologous antigen into a cell. In some embodiments, the vector is introduced into the cell under conditions in which one or more peptide antigens, including, in some cases, one or more peptide antigens of the expressed heterologous protein, are expressed by the cell, processed and presented on the surface of the cell in the context of a major histocompatibility complex (MHC) molecule.

Generally, the cell to which the vector is contacted is a cell that expresses MHC, i.e., MHC-expressing cells. The cell may be one that normally expresses an MHC on the cell surface, that is induced to express and/or upregulate expression of MHC on the cell surface or that is engineered to express an MHC molecule on the cell surface. In some embodiments, the MHC contains a polymorphic peptide binding site or binding groove that can, in some cases, complex with peptide antigens of polypeptides, including peptide antigens processed by the cell machinery. In some cases, MHC molecules may be displayed or expressed on the cell surface, including as a complex with peptide, i.e., MHC-peptide complex, for presentation of an antigen in a conformation recognizable by TCRs on T cells, or other peptide binding molecules.

In some embodiments, the cell is a nucleated cell. In some embodiments, the cell is an antigen-presenting cell. In some embodiments, the cell is a macrophage, dendritic cell, B cell, endothelial cell or fibroblast. In some embodiments, the cell is an endothelial cell, such as an endothelial cell line or primary endothelial cell. In some embodiments, the cell is a fibroblast, such as a fibroblast cell line or a primary fibroblast cell.

In some embodiments, the cell is an artificial antigen presenting cell (aAPC). Typically, aAPCs include features of natural APCs, including expression of an MHC molecule, stimulatory and costimulatory molecule(s), Fc receptor, adhesion molecule(s) and/or the ability to produce or secrete cytokines (e.g., IL-2). Normally, an aAPC is a cell line that lacks expression of one or more of the above, and is generated by introduction (e.g., by transfection or transduction) of one or more of the missing elements from among an MHC molecule, a low affinity Fc receptor (CD32), a high affinity Fc receptor (CD64), one or more of a co-stimulatory signal (e.g., CD7, B7-1 (CD80), B7-2 (CD86), PD-L1, PD-L2, 4-1BBL, OX40L, ICOS-L, ICAM, CD30L, CD40, CD70, CD83, HLA-G, MICA, MICB, HVEM, lymphotoxin beta receptor, ILT3, ILT4, 3/TR6 or a ligand of B7-H3; or an antibody that specifically binds to CD27, CD28, 4-1BB, OX40, CD30, CD40, PD-1, ICOS, LFA-1, CD2, CD7, LIGHT, NKG2C, B7-H3, Toll ligand receptor or a ligand of CD83), a cell adhesion molecule (e.g., ICAM-1 or LFA-3) and/or a cytokine (e.g., IL-2, IL-4, IL-6, IL-7, IL-10, IL-12, IL-15, IL-21, interferon-alpha (IFN.alpha.), interferon-beta (IFN.beta.), interferon-gamma (IFN.gamma.), tumor necrosis factor-alpha (TNF.alpha.), tumor necrosis factor-beta (TNF.beta.), granulocyte macrophage colony stimulating factor (GM-CSF), and granulocyte colony stimulating factor (GCSF)). In some cases, an aAPC does not normally express an MHC molecule, but may be engineered to express an MHC molecule or, in some cases, is or may be induced to express an MHC molecule, such as by stimulation with cytokines. In some cases, aAPCs also may be loaded with a stimulatory ligand, which may include, for example, an anti-CD3 antibody, an anti-CD28 antibody or an anti-CD2 antibody. An exemplary cell line that may be used as a backbone for generating an aAPC is a K562 cell line or a fibroblast cell line. Various aAPCs are known in the art, see e.g., U.S. Pat. No. 8,722,400, published application No. US2014/0212446; Butler and Hirano (2014) Immunol Rev. 257:10. 1111/imr.12129; Suhoshki et al. (2007)Mol. Ther. 15:981-988).

It is well within the level of a skilled artisan to determine or identify the particular MHC or allele expressed by a cell. In some embodiments, prior to contacting cells with a vector, expression of a particular MHC molecule may be assessed or confirmed, such as by using an antibody specific for the particular MHC molecule. Antibodies to MHC molecules are known in the art, such as any described below.

In some embodiments, the cells may be chosen to express an MHC allele of a desired MHC restriction. In some embodiments, the MHC typing of cells, such as cell lines, are well-known in the art. In some embodiments, the MHC typing of cells, such as primary cells obtained from a subject, may be determined using procedures well-known in the art, such as by performing tissue typing using molecular haplotype assays (BioTest ABC SSPtray, BioTest Diagnostics Corp., Denville, N.J.; SeCore Kits, Life Technologies, Grand Island, N.Y.). In some cases, it is well within the level of a skilled artisan to perform standard typing of cells to determine the HLA genotype, such as by using sequence-based typing (SBT) (Adams et al. (2004) J. Transl. Med., 2:30; Smith (2012) Methods Mol Biol., 882:67-86). In some cases, the HLA typing of cells, such as fibroblast cells, are known. For example, the human fetal lung fibroblast cell line MRC-5 is HLA-A*02:01, A29, B13, B44 Cw7 (C*0702); the human foreskin fibroblast cell line Hs68 is HLA-A1, A29, B8, B44, Cw7, Cw16; and the WI-38 cell line is A*68:01, B*08:01, (Solache et al. (1999) J Immunol, 163:5512-5518; Ameres et al. (2013) PloS Pathog. 9:e1003383). The human transfectant fibroblast cell line M1DR1/Ii/DM express HLA-DR and HLA-DM (Karakikes et al. (2012) FASEB J., 26:4886-96).

In some embodiments, the cells to which the vector is contacted or introduced are cells that are engineered or transfected to express an MHC molecule. In some embodiments, cell lines may be prepared by genetically modifying a parental cells line. In some embodiments, the cells are normally deficient in the particular MHC molecule and are engineered to express such particular MHC molecule. In some embodiments, the cells are genetically engineered using recombinant DNA techniques.

In some embodiments, the stable MHC-peptide complexes described herein are used to detect T cells that bind a stable MHC-peptide complex. In some embodiments, the stable MHC-peptide complexes described herein are used to monitor T cell response in a subject, for example, by detecting the amount and/or percentage of T cells (e.g., CD8+ T cells) that specifically and/or selectively bind to the MHC-peptide complexes that are fluorescently labeled. Methods of generating, labeling, and using MHC-peptide complexes (e.g., MHC-peptide tetramers) for detecting MHC-peptide complex-specific T cells are well-known in the art. Additional description can be found in, for example, U.S. Pat. Nos. 7,776,562; 8,268,964; and U.S. Pat. Publ. 2019/0085048.

### IV. Immunogenic Compositions

In some aspects, provided herein are pharmaceutical compositions (e.g., a vaccine composition) comprising a MAGEA1 immunogenic peptide and/or a nucleic acid encoding a MAGEA1 immunogenic peptide and an adjuvant. In some aspects, provided herein are pharmaceutical compositions (e.g., a vaccine composition) comprising a stable MHC-peptide complex comprising a MAGEA1 immunogenic peptide in the context of a MHC molecule and an adjuvant. In some embodiments, the composition includes a combination of multiple (e.g., two or more) MAGEA1 immunogenic peptides or nucleic acids and an adjuvant. In some embodiments, the composition includes a combination of multiple (e.g., two or more) stable MHC-peptide complexes comprising a MAGEA1 immunogenic peptide in the context of a MHC molecule and an adjuvant. In some embodiments, the compositions described above further comprises a pharmaceutically acceptable carrier.

The pharmaceutical compositions disclosed herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; or (2) parenteral administration, for example, by subcutaneous, intramuscular, intravenous or epidural injection as, for example, a sterile solution or suspension, or sustained-release formulation.

Methods of preparing these formulations or compositions include the step of bringing into association a MAGEA1 immunogenic peptide and/or nucleic acid described herein with the adjuvant, carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions suitable for parenteral administration comprise MAGEA1 immunogenic peptides and/or nucleic acids described herein in combination with a adjuvant, as well as one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

Regardless of the route of administration selected, the agents provided herein, which may be used in a suitable hydrated form, and/or the pharmaceutical compositions disclosed herein, are formulated into pharmaceutically-acceptable dosage forms by conventional methods known to those of skill in the art.

In some embodiments, the pharmaceutical composition described, when administered to a subject, can elicit an immune response against a cell that is infected by MAGEA1. Such pharmaceutical compositions may be useful as vaccine compositions for prophylactic and/or therapeutic treatment of disorders characterized by MAGEA1 expression.

In some embodiments, the pharmaceutical composition further comprises a physiologically acceptable adjuvant. In some embodiments, the adjuvant employed provides for increased immunogenicity of the pharmaceutical composition. Such a further immune response stimulating compound or adjuvant may be (i) admixed to the pharmaceutical composition according to the invention after reconstitution of the peptides and optional emulsification with an oil-based adjuvant as defined above, (ii) may be part of the reconstitution composition of the invention defined above, (iii) may be physically linked to the peptide(s) to be reconstituted or (iv) may be administered separately to the subject, mammal or human, to be treated. The adjuvant may be one that provides for slow release of antigen (e.g., the adjuvant may be a liposome), or it may be an adjuvant that is immunogenic in its own right thereby functioning synergistically with antigens (i.e., antigens present in the MAGEA1 immunogenic peptide). For example, the adjuvant may be a known adjuvant or other substance that promotes antigen uptake, recruits immune system cells to the site of administration, or facilitates the immune activation of responding lymphoid cells. Adjuvants include, but are not limited to, immunomodulatory molecules (e.g., cytokines), oil and water emulsions, aluminum hydroxide, glucan, dextran sulfate, iron oxide, sodium alginate, Bacto-Adjuvant, synthetic polymers such as poly amino acids and co-polymers of amino acids, saponin, paraffin oil, and muramyl dipeptide. In some embodiments, the adjuvant is Adjuvant 65, α-GalCer, aluminum phosphate, aluminum hydroxide, calcium phosphate, β-Glucan Peptide, CpG DNA, GM-CSF, GPI-0100, IFA, IFN-γ, IL-17, lipid A, lipopolysaccharide, Lipovant, Montanide, N-acetyl-muramyl-L-alanyl-D-isoglutamine, Pam3CSK4, quil A, trehalose dimycolate or zymosan.

In some embodiments, the adjuvant is an immunomodulatory molecule. For example, the immunomodulatory molecule may be a recombinant protein cytokine, chemokine, or immunostimulatory agent or nucleic acid encoding cytokines, chemokines, or immunostimulatory agents designed to enhance the immunologic response.

Examples of immunomodulatory cytokines include interferons (e.g., IFNα, IFNβ and IFNγ), interleukins (e.g., IL-1, IL-2, IL-3, IL-4, IL-5, IL-6, IL-7, IL-8, IL-9, IL-10, IL-12, IL-17 and IL-20), tumor necrosis factors (e.g., TNFα and TNFβ), erythropoietin (EPO), FLT-3 ligand, gIp10, TCA-3, MCP-1, MIF, MIP-1.alpha., MIP-1β, Rantes, macrophage colony stimulating factor (M-CSF), granulocyte colony stimulating factor (G-CSF), and granulocyte-macrophage colony stimulating factor (GM-CSF), as well as functional fragments of any of the foregoing.

In some embodiments, an immunomodulatory chemokine that binds to a chemokine receptor, i.e., a CXC, CC, C, or CX3C chemokine receptor, also may be included in the compositions provided here. Examples of chemokines include, but are not limited to, Mip1α, Mip-1β, Mip-3α (Larc), Mip-3β, Rantes, Hcc-1, Mpif-1, Mpif-2, Mcp-1, Mcp-2, Mcp-3, Mcp-4, Mcp-5, Eotaxin, Tarc, Elc, 1309, IL-8, Gcp-2 Gro-α, Gro-β, Gro-γ, Nap-2, Ena-78, Gcp-2, Ip-10, Mig, I-Tac, Sdf-1, and Bca-1 (Blc), as well as functional fragments of any of the foregoing.

In some embodiments, the composition comprises a nucleic acid encoding an MAGEA1 immunogenic polypeptide described herein, such as a DNA molecule encoding a MAGEA1 immunogenic peptide. In some embodiments the composition comprises an expression vector comprising an open reading frame encoding a MAGEA1 immunogenic peptide.

When taken up by a cell (e.g., host cell, an antigen-presenting cell (APC) such as a dendritic cell, macrophage, etc.), a DNA molecule may be present in the cell as an extrachromosomal molecule and/or may integrate into the chromosome. DNA may be introduced into cells in the form of a plasmid which may remain as separate genetic material. Alternatively, linear DNAs that may integrate into the chromosome may be introduced into the cell. Optionally, when introducing DNA into a cell, reagents which promote DNA integration into chromosomes may be added.

### V. Binding Proteins

In some aspects, a binding moiety that binds a peptide described herein and/or a stable MHC-peptide complex described herein, are provided. For example, binding proteins like T cell receptors (TCRs), antibodies, and the like that specifically and/or selectively bind to the peptide and/or the stable MHC-peptide complex, such as with a K_{d} less than or equal to about 10⁻⁴ M (e.g., about 10⁻⁴, 10⁻⁵, 10⁻⁶, 10⁻⁷, about 10⁻⁸, about 10⁻⁹, about 10⁻¹⁰, about 10⁻¹¹, about 10⁻¹², about 10⁻¹³, about 10⁻¹⁴, etc.), are provided.

In an aspect encompassed by the present invention, provided herein are binding proteins that bind (e.g., specifically and/or selectively) to a peptide-MHC (pMHC) complex comprising a MAGEA1 immunogenic peptide in the context of an MHC molecule (e.g., an MHC class I molecule). In some embodiments, the binding protein is capable of binding (e.g., specifically and/or selectively) to a MAGEA1 peptide-MHC (pMHC) complex with a K_{d} less than or equal to about 5×10⁻⁴ M, less than or equal to about 1×10⁻⁴ M, less than or equal to about 5×10⁻⁵ M, less than or equal to about 1×10⁵ M, less than or equal to about 5×10⁻⁶ M, less than or equal to about 1×10⁻⁶ M, less than or equal to about 5×10⁻⁷ M, less than or equal to about 1×10⁻⁷ M, less than or equal to about 5×10⁻⁸ M, less than or equal to about 1×10⁻⁸ M, less than or equal to about 5×10⁻⁹ M, less than or equal to about 1×10⁻⁹ M, less than or equal to about 5×10-10 M, less than or equal to about 1×10-10 M, less than or equal to about 5×10-11 M, less than or equal to about 1×10-11 M, less than or equal to about 5×10-12 M, less than or equal to about 1×10-12 M, or any range in between, inclusive, such as between about 1-50 micromolar, 1-100 micromolar, 0.1-500 micromolar, and the like. In some embodiments, the MHC molecule comprises an MHC alpha chain that is an HLA serotype selected from the group consisting of HLA-A*02, HLA-A*03, HLA-A*01, HLA-A*11, HLA-A*24, HLA-B*07, HLA-C*07, HLA-C*01, HLA-C*02, HLA-C*03, HLA-C*04, HLA-C*05, HLA-C*06, HLA-C*08, HLA-C*12, HLA-C*14, HLA-C*15, HLA-C*16, HLA-C*17, and HLA-C*18, optionally wherein the HLA allele is selected from the group consisting of HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:12, HLA-A*02:13, HLA-A*02:14, HLA-A*02:16, HLA-A*02:17, HLA-A*02:19, HLA-A*02:20, HLA-A*02:22, HLA-A*02:24, HLA-A*02:30, HLA-A*02:42, HLA-A*02:53, HLA-A*02:60, HLA-A*02:74 allele, HLA-A*03:01, HLA-A*03:02, HLA-A*03:05, HLA-A*03:07, HLA-A*01:01, HLA-A*01:02, HLA-A*01:03, HLA-A*01:16 allele, HLA-A*11:01, HLA-A*11:02, HLA-A*11:03, HLA-A*11:04, HLA-A*11:05, HLA-A*11:19 allele, HLA-A*24:02, HLA-A*24:03, HLA-A*24:05, HLA-A*24:07, HLA-A*24:08, HLA-A*24:10, HLA-A*24:14, HLA-A*24:17, HLA-A*24:20, HLA-A*24:22, HLA-A*24:25, HLA-A*24:26, HLA-A*24:58 allele, HLA-B*07:02, HLA-B*07:04, HLA-B*07:05, HLA-B*07:09, HLA-B*07:10, HLA-B*07:15, HLA-B*07:21, HLA-C*07:02, HLA-C*07:01, HLA-C*04:01, HLA-C*06:02, HLA-C*03:04, HLA-C*05:01, HLA-C*16:01, HLA-C*02:02, HLA-C*03:03, HLA-C*12:03, HLA-C*08:02, HLA-C*01:02, HLA-C*17:01, HLA-C*15:02, HLA-C*14:02, HLA-C*12:02, HLA-C*07:04, HLA-C*08:01, HLA-C*03:02, HLA-C*18:01, HLA-C*15:05, HLA-C*16:02, HLA-C*08:04, HLA-C*03:05, and HLA-C*14:03 allele. In some embodiments, the HLA serotype is HLA-A*02 and/or the HLA allele is HLA-A*02:01 allele. In some embodiments, the binding proteins provided herein are genetically engineered, isolated, and/or purified.

In some embodiments, the binding proteins have a higher binding affinity to the MAGEA1 peptide-MHC (pMHC) than does a known T-cell receptor (e.g., a TCR from van Kunert et al. (2016) J. Immunol. 197:2541-2552 or others described herein). For example, the binding proteins may have at least 1.2 fold, 1.5 fold, 1.8 fold, 2.0 fold, 2.2 fold, 2.5 fold, 2.8 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 11 fold, 12 fold, 13 fold, 14 fold, 15 fold, 16 fold, 17 fold, 18 fold, 19 fold, 20 fold, 25 fold, 30 fold, 35 fold, 40 fold, 45 fold, 50 fold, 60 fold, 70 fold, 80 fold, 90 fold, 100 fold, 1000 fold, 5000 fold, 10000 fold, 50000 fold, 100000 fold, 500000 fold, 1000000 fold, or more, or any range in between, inclusive, such as 1.2 fold to 2 fold, higher binding affinity to the MAGEA1 peptide-MHC (pMHC) than does a known T-cell receptor.

In some embodiments, the binding protein induces higher T cell expansion, cytokine release, and/or cytotoxic killing than does a known T-cell receptor when contacted with target cells with expression of MAGEA1 at a certain level or below (e.g., see the Examples section for representative cell lines expressing MAGEA1 at varying levels). For example, in some embodiments of any aspect described herein, MAGEA1 level can be expressed in terms of transcripts per million and may be, for example, less than or equal to about 1,000 transcript per million transcripts (TPM), 950 TPM, 900 TPM, 850 TPM, 800 TPM, 750 TPM, 700 TPM, 650 TPM, 600 TPM, 550 TPM, 500 TPM, 450 TPM, 400 TPM, 350 TPM, 300 TPM, 250 TPM, 200 TPM, 150 TPM, 100 TPM, 95 TPM, 90 TPM, 85 TPM, 80 TPM, 75 TPM, 70 TPM, 65 TPM, 60 TPM, 55 TPM, 50 TPM, 45 TPM, 40 TPM, 35 TPM, 34 TPM, 33 TPM, 32 TPM, 31 TPM, 30 TPM, 29 TPM, 28 TPM, 27 TPM, 26 TPM, 25 TPM, 24 TPM, 23 TPM, 22 TPM, 21 TPM, 20 TPM, 19 TPM, 18 TPM, 17 TPM, 16 TPM, 15 TPM, 14 TPM, 13 TPM, 12 TPM, 11 TPM, 10 TPM, 9 TPM, 8 TPM, 7 TPM, 6 TPM, 5 TPM, 4 TPM, 3 TPM, 2 TPM, and 1 TPM, or any range in between, inclusive, such as less than or equal to about 1,000 TPM to less than or equal to about 35 TPM). In some embodiments, the low MAGEA1 expression level is termed “heterozygous expression” meaning between about 1 TPM and about 35 TPM, or any range in between, inclusive, such as 32 TPM or 1-32 TPM. A higher expression is 36 TPM and higher. As described further herein, TPM is measured according to well-known techniques, such as RNA-Seq, and gene expression TPM data are well-known in the art for a variety of cell lines, tissue types, and the like (see, for example, the Broad Institute Cancer Cell Line Encyclopedia (CCLE) on the World Wide Web at portals.broadinstitute.org). In some embodiment, the binding protein induces at least 1.2 fold, 1.5 fold, 1.8 fold, 2.0 fold, 2.2 fold, 2.5 fold, 2.8 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 11 fold, 12 fold, 13 fold, 14 fold, 15 fold, 16 fold, 17 fold, 18 fold, 19 fold, 20 fold, 25 fold, 30 fold, 35 fold, 40 fold, 45 fold, 50 fold, 60 fold, 70 fold, 80 fold, 90 fold, 100 fold, 1000 fold, or more, or any range in between, inclusive, such as 1.2 fold to 2 fold, increase in T cell expansion, cytokine release, and/or cytotoxic killing than does a known T-cell receptor (e.g., a comparator TCR described herein) when contacted with target cells expressing MAGEA1 peptide epitope, such as with heterozygous expression of MAGEA1 peptide epitope.

In some embodiments, the expression of MAGEA1 is detected using RNA-sequencing (RNA-seq). RNA-seq generally comprises the following steps: obtaining a sample containing genetic material, isolating total RNA from the sample obtained, preparing an amplified cDNA library from the total RNA, sequencing the amplified cDNA library, and analyzing and profiling the amplified cDNA to assess the expression level of different transcripts. The sample can be a population of cells, a tissue sample, a bioposy sample, a cell culture, or a single cell. Total RNA can be isolated from the biological sample using any method known in the art. In certain embodiments, total RNA is extracted from plasma. Plasma RNA extraction is described in Enders et al., “The Concentration of Circulating Corticotropin-Releasing Homer mRNA in Material Plasma Is Inclined in Preclampsia,” Clinr. As described therein, the plasma collected after the centrifugation step is mixed with Trizol LS reagent (Invitrogen) and chloroform. The mixture is centrifuged and the aqueous layer is transferred to a new tube. Ethanol is added to this aqueous layer. The mixture is then placed in an RNeasy mini column (Qiagen) and processed according to the manufacturer's recommendations.

In some embodiments, RNA-seq described herein includes the step of preparing amplified cDNA from total RNA. For example, cDNA is prepared and the isolated RNA sample is randomly amplified without dilution, or the mixture of genetic material in the isolated RNA is dispersed into individual reaction samples. In certain embodiments, amplification is initiated randomly at the 3′ end and throughout the entire transcriptome in the sample to amplify both mRNA and non-polyadenylated transcripts. In this way, double-stranded cDNA amplification products are optimized for the generation of sequencing libraries for next generation sequencing platforms. A kit suitable for amplification of cDNA by the method encompassed by the present invention includes, for example, Ovation® RNA-Seq System.

In some embodiments, RNA-seq described herein includes the step of sequencing the amplified cDNA. Any known sequencing method can be used to sequence the amplified cDNA mixture including the single molecule sequencing method. In certain embodiments, the amplified cDNA is sequenced by whole transcriptome shotgun sequencing. Whole transcriptome shotgun sequencing can be performed using various next generation sequencing platforms such as Illumina® Genome Analyzer platform, ABI SOLID™ Sequencing platform, or Life Science's 454 Sequencing platform.

In some embodiments, RNA-seq described herein further comprises performing digital counting and analysis on the cDNA. The number of amplified sequences for each transcript in the amplified sample can be quantified by sequence reading (one reading per amplified strand). In some embodiments, transcript per million (TPM) is used to quantify the expression level of a particular transcript. TPM may be calculated as shown in Wagner et al. (2012) Theory in Biosciences 131:281-285, the content of which is incorporated by reference herein in its entirety.

In certain embodiments, the binding proteins recognize a MAGEA1 immunogenic peptide in a complex with MHC molecules, such as particular HLA molecules having particular HLA alpha chain alleles. For example, binding proteins listed in Table 2A were identified as binders of MAGEA1 immunogenic peptides in association with an MHC whose alpha chain had an HLA-A*02 serotype, such as that encoded by an HLA-A*02:01 allele, as described further in the Examples section. In some embodiments, the binding proteins recognize a complex of MAGEA1 immunogenic peptide and an MHC molecule, wherein the MHC molecule comprises an MHC alpha chain that is an HLA serotype selected from the group consisting of HLA-A*02, HLA-A*03, HLA-A*01, HLA-A*11, HLA-A*24, HLA-B*07, HLA-C*07, HLA-C*01, HLA-C*02, HLA-C*03, HLA-C*04, HLA-C*05, HLA-C*06, HLA-C*08, HLA-C*12, HLA-C*14, HLA-C*15, HLA-C*16, HLA-C*17, and HLA-C*18, optionally wherein the HLA allele is selected from the group consisting of HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:12, HLA-A*02:13, HLA-A*02:14, HLA-A*02:16, HLA-A*02:17, HLA-A*02:19, HLA-A*02:20, HLA-A*02:22, HLA-A*02:24, HLA-A*02:30, HLA-A*02:42, HLA-A*02:53, HLA-A*02:60, HLA-A*02:74 allele, HLA-A*03:01, HLA-A*03:02, HLA-A*03:05, HLA-A*03:07, HLA-A*01:01, HLA-A*01:02, HLA-A*01:03, HLA-A*01:16 allele, HLA-A*11:01, HLA-A*11:02, HLA-A*11:03, HLA-A*11:04, HLA-A*11:05, HLA-A*11:19 allele, HLA-A*24:02, HLA-A*24:03, HLA-A*24:05, HLA-A*24:07, HLA-A*24:08, HLA-A*24:10, HLA-A*24:14, HLA-A*24:17, HLA-A*24:20, HLA-A*24:22, HLA-A*24:25, HLA-A*24:26, HLA-A*24:58 allele, HLA-B*07:02, HLA-B*07:04, HLA-B*07:05, HLA-B*07:09, HLA-B*07:10, HLA-B*07:15, HLA-B*07:21, HLA-C*07:02, HLA-C*07:01, HLA-C*04:01, HLA-C*06:02, HLA-C*03:04, HLA-C*05:01, HLA-C*16:01, HLA-C*02:02, HLA-C*03:03, HLA-C*12:03, HLA-C*08:02, HLA-C*01:02, HLA-C*17:01, HLA-C*15:02, HLA-C*14:02, HLA-C*12:02, HLA-C*07:04, HLA-C*08:01, HLA-C*03:02, HLA-C*18:01, HLA-C*15:05, HLA-C*16:02, HLA-C*08:04, HLA-C*03:05, and HLA-C*14:03 allele. In some embodiments, the MAGEA1 immunogenic peptides are derived from a human MAGEA1 protein and/or a MAGEA1 protein shown in Table 3. In some embodiments, one or more MAGEA1 immunogenic peptides are administered alone or in combination with an adjuvant.

In some embodiments, the binding proteins do not bind to a peptide-MHC (pMHC) complex, optionally wherein the peptide is derived from an “off-target” described herein.

In some embodiments, the binding protein does not bind to an “off-target” described herein complexed with an MHC peptide-MHC (pMHC) complex.

In some embodiments, the binding proteins provided herein include (e.g., comprise, consist essentially of, or consist of): a) a TCR alpha chain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR alpha chain sequence selected from the group consisting of the TCR alpha sequences listed in Table 2; and/or b) a TCR beta chain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR beta chain sequence selected from the group consisting of the TCR beta chain sequences listed in Table 2.

In some embodiments, the binding proteins provided herein include (e.g., comprise, consist essentially of, or consist of): a) a TCR alpha chain sequence selected from the group consisting of the TCR alpha chain sequences listed in Table 2; and/or b) a TCR beta chain sequence selected from the group consisting of the TCR beta chain sequences listed in Table 2.

In some embodiments, the binding proteins provided herein include (e.g., comprise, consist essentially of, or consist of): a) a TCR alpha chain variable (V_{α}) domain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR alpha chain variable (V_{α}) domain sequence selected from the group consisting of the TCR V_{α} domain sequences listed in Table 2; and/or b) a TCR beta chain variable (V_{β}) domain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR beta chain variable (V_{β}) domain sequence selected from the group consisting of the TCR V_{β} domain sequences listed in Table 2.

In some embodiments, the binding proteins provided herein include (e.g., comprise, consist essentially of, or consist of): a) a TCR alpha chain variable (V_{α}) domain sequence selected from the group consisting of the TCR V_{α} domain sequences listed in Table 2; and/or b) a TCR beta chain variable (V_{β}) domain sequence selected from the group consisting of the TCR V_{β} domain sequences listed in Table 2.

In some embodiments, the binding proteins provided herein include (e.g., comprise, consist essentially of, or consist of at least one (e.g., one, two or three, such as CDR3 alone or in combination with a CDR1 and CDR2)) TCR alpha chain complementarity determining region (CDR) sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR alpha chain CDR sequence selected from the group consisting of the TCR alpha chain CDR sequences listed in Table 2. CDR3 is believed to be the main CDR responsible for recognizing processed antigen and CDR1 and CDR2 mainly interact with the MHC, so, in some embodiments, binding protein comprising a CDR3 alone from a TCR alpha chain and/or a CDR3 alone from a TCR beta chain listed in Table 2, each CDR3 having a sequence homology as recited in this paragraph, are provided.

In some embodiments, the binding proteins provided herein may also include (e.g., comprise, consist essentially of, or consist of at least one (e.g., one, two or three, such as CDR3 alone or in combination with a CDR1 and CDR2)) TCR beta chain complementarity determining region (CDR) sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR beta chain CDR sequence selected from the group consisting of the TCR beta chain CDR sequences listed in Table 2. As described above, CDR3 is believed to be the main CDR responsible for recognizing processed antigen and CDR1 and CDR2 mainly interact with the MHC, so, in some embodiments, binding protein comprising a CDR3 alone from a TCR beta chain and/or a CDR3 alone from a TCR alpha chain listed in Table 2, each CDR3 having a sequence homology as recited in this paragraph, are provided.

In some embodiments, the binding proteins provided herein include (e.g., comprise, consist essentially of, or consist of at least one (e.g., one, two or three)) TCR alpha chain complementarity determining region (CDR) listed in Table 2.

In some embodiments, the binding proteins provided herein may also include (e.g., comprise, consist essentially of, or consist of at least one (e.g., one, two or three)) TCR beta chain complementarity determining region (CDR) listed in Table 2.

In some embodiments, the binding proteins provided herein include (e.g., comprise, consist essentially of, or consist of) a TCR alpha chain constant region (C_{α}) sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR Cα sequence listed in Table 2.

In some embodiments, the binding proteins provided herein may also include (e.g., comprise, consist essentially of, or consist of) a TCR beta chain constant region (C_{β}) sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR CB sequence listed in Table 2.

In some embodiments, the binding proteins provided herein include (e.g., comprise, consist essentially of, or consist of) a TCR alpha chain constant region (C_{α}) sequence selected from the group consisting of the TCR C_{α} sequences listed in Table 2.

In some embodiments, the binding proteins provided herein may also include (e.g., comprise, consist essentially of, or consist of) a TCR beta chain constant region (C_{β}) sequence selected from the group consisting of the TCR C_{β} sequences listed in Table 2.

TABLE 2
TCR sequences recognizing a MAGEA1 antigen
Table 2A
TCR sequences recognizing a MAGEA1 antigen presented by HLA serotype HLA-A*02

MAGEA1-278-1479 WT sequence
Alpha chain:
TRAV20/TRAJ26/TRAC
Alpha chain DNA sequence
ATGGAAAAAATGCTCGAGTGCGCCTTCATCGTGCTTTGGCTGCAGCTCGGATGGC
TGAGCGGAGAGGATCAAGTGACACAGTCTCCCGAGGCTCTGAGGCTGCAAGAGG
GCGAAAGCAGCTCCCTGAATTGCAGCTACACCGTGTCTGGCCTGAGGGGCCTG
TTTTGGTACAGACAAGACCCTGGCAAGGGACCCGAGTTCCTGTTCACACTGTAC
TCTGCCGGCGAAGAAAAAGAGAAAGAGCGCCTGAAAGCAACCCTGACCAAGA
AAGAGAGCTTCCTGCACATCACAGCCCCTAAGCCAGAGGACAGCGCTACTTACC
TGTGTGCCGTTTCATACGGCCAGAATTTCGTTTTTGGTCCCGGAACCAGATTG
TCCGTGCTGCCCTatatccagaaccctgaccctgccgtgtaccagctgagagactctaaatccagtgacaagtctgtctgcc
tattcaccgattttgattctcaaacaaatgtgtcacaaagtaaggattctgatgtgtatatcacagacaaaactgtgctagacatgaggtct
atggacttcaagagcaacagtgctgtggcctggagcaacaaatctgactttgcatgtgcaaacgccttcaacaacagcattattccagaa
gacaccttcttccccagcccagaaagttcctgtgatgtcaagctggtcgagaaaagctttgaaacagatacgaacctaaactttcaaaac
ctgtcagtgattgggttccgaatcctcctcctgaaagtggccgggtttaatctgctcatgacgctgcggctgtggtccagc (SEQ ID
NO: 4)
Alpha chain protein sequence
MEKMLECAFIVLWLQLGWLSGEDQVTQSPEALRLQEGESSSLNCSYTVSGLRGLFW
YRQDPGKGPEFLFTLYSAGEEKEKERLKATLTKKESFLHITAPKPEDSATYLCAVSY
GQNFVFGPGTRLSVLPYiqnpdpavyqlrdskssdksvclftdfdsqtnvsqskdsdvyitdktvldmrsmdfksn
savawsnksdfacanafnnsiipedtffpspesscdvklveksfetdtnlnfqnlsvigfrilllkvagfnllmtlrlwss (SEQ ID
NO: 5)
Beta chain:
TRBV5-8/TRBJ1-1/TRBC1
Beta chain DNA sequence
ATGGGACCCAGGCTCCTCTTCTGGGCACTGCTTTGTCTCCTCGGAACAGGCCCAG
TGGAGGCTGGAGTCACACAAAGTCCCACACACCTGATCAAAACGAGAGGACAGC
AAGCGACTCTGAGATGCTCTCCTATCTCTGGGCACACCAGTGTGTACTGGTACC
AACAGGCCCTGGGTCTGGGCCTCCAGTTCCTCCTTTGGTATGACGAGGGTGAAG
AGAGAAACAGAGGAAACTTCCCTCCTAGATTTTCAGGTCGCCAGTTCCCTAATTA
TAGCTCTGAGCTGAATGTGAACGCCTTGGAGCTGGAGGACTCGGCCCTGTATCTC
TGTGCTTCCTCACTTGGGCAATTGAACACAGAGGCATTCTTTGGACAAGGCA
CCAGACTCACAGTTGTAGaggacctgaacaaggtgttcccacccgaggtcgctgtgtttgagccatcagaagcaga
gatctcccacacccaaaaggccacactggtgtgcctggccacaggcttcttccctgaccacgtggagctgagctggtgggtgaatgg
gaaggaggtgcacagtggggtcagcacggacccgcagcccctcaaggagcagcccgccctcaatgactccagatactgcctgagc
agccgcctgagggtctcggccaccttctggcagaacccccgcaaccacttccgctgtcaagtccagttctacgggctctcggagaatg
acgagtggacccaggatagggccaaacccgtcacccagatcgtcagcgccgaggcctggggtagagcagactgtggctttacctcg
gtgtcctaccagcaaggggtcctgtctgccaccatcctctatgagatcctgctagggaaggccaccctgtatgctgtgctggtcagcgc
ccttgtgttgatggccatggtcaagagaaaggatttc (SEQ ID NO: 6)
Beta chain protein sequence
MGPRLLFWALLCLLGTGPVEAGVTQSPTHLIKTRGQQATLRCSPISGHTSVYWYQQ
ALGLGLQFLLWYDEGEERNRGNFPPRFSGRQFPNYSSELNVNALELEDSALYLCASS
LGQLNTEAFFGQGTRLTVVEdlnkvfppevavfepseaeishtqkatlvclatgffpdhvelswwvngkevhsg
vstdpqplkeqpalndsryclssrlrvsatfwqnprnhfrcqvqfyglsendewtqdrakpvtqivsaeawgradcgftsvsyqq
gvlsatilyeillgkatlyavlvsalvlmamvkrkdf (SEQ ID NO: 7)
MAGEA1-278-1479 MGTM codon optimized sequence (also known as clone “MAGE-
A1-1479,” “TCR 1479”, “1479”, TCR expressed by “TSC-204-A02”, and TCR
expressed by ″TSC-204-A0201″)
Alpha chain:
TRAV20/TRAJ26/MGTM modified TRAC
Alpha chain DNA sequence
ATGGAAAAAATGCTCGAGTGCGCCTTCATCGTGCTTTGGCTGCAGCTCGGATGGC
TGAGCGGAGAGGATCAAGTGACACAGTCTCCCGAGGCTCTGAGGCTGCAAGAGG
GCGAAAGCAGCTCCCTGAATTGCAGCTACACCGTGTCTGGCCTGAGGGGCCTG
TTTTGGTACAGACAAGACCCTGGCAAGGGACCCGAGTTCCTGTTCACACTGTAC
TCTGCCGGCGAAGAAAAAGAGAAAGAGCGCCTGAAAGCAACCCTGACCAAGA
AAGAGAGCTTCCTGCACATCACAGCCCCTAAGCCAGAGGACAGCGCTACTTACC
TGTGTGCCGTTTCATACGGCCAGAATTTCGTTTTTGGTCCCGGAACCAGATTG
TCCGTGCTGCCCTacatccagaaccccgaccccgccgtgtaccagctgagggactccaagtccagcgacaagagcgtgt
gtctgtttacggacttcgacagccagaccaacgtgagtcaaagcaaggacagcgacgtctacataacggataagaccgtgctggaca
tgcggagcatggacttcaagagcaacagcgccgtggcctggtccaacaagagcgacttcgcctgcgccaacgccttcaacaacagc
atcatccccgaggacaccttcttccccagcagcgacgtgccctgcgacgtgaaactggtggagaagtccttcgagacagacaccaat
ctgaactttcagaacctgctggtgatcgtgctgcggattctgctgctgaaagtggccggcttcaatctgctgatgaccctgcggctgtgg
agcagc (SEQ ID NO: 8)
Alpha chain protein sequence
MEKMLECAFIVLWLQLGWLSGEDQVTQSPEALRLQEGESSSLNCSYTVSGLRGLFW
YRQDPGKGPEFLFTLYSAGEEKEKERLKATLTKKESFLHITAPKPEDSATYLCAVSY
GQNFVFGPGTRLSVLPYiqnpdpavyqlrdskssdksvclftdfdsqtnvsqskdsdvyitdktvldmrsmdfksn
savawsnksdfacanafnnsiipedtffpssdvpcdvklveksfetdtnlnfqnllvivlrilllkvagfnllmtlrlwss (SEQ ID
NO: 9)
Beta chain:
TRBV5-8/TRBJ1-1/MGTM modified TRBC
Beta chain DNA sequence
ATGGGACCCAGGCTCCTCTTCTGGGCACTGCTTTGTCTCCTCGGAACAGGCCCAG
TGGAGGCTGGAGTCACACAAAGTCCCACACACCTGATCAAAACGAGAGGACAGC
AAGCGACTCTGAGATGCTCTCCTATCTCTGGGCACACCAGTGTGTACTGGTACC
AACAGGCCCTGGGTCTGGGCCTCCAGTTCCTCCTTTGGTATGACGAGGGTGAAG
AGAGAAACAGAGGAAACTTCCCTCCTAGATTTTCAGGTCGCCAGTTCCCTAATTA
TAGCTCTGAGCTGAATGTGAACGCCTTGGAGCTGGAGGACTCGGCCCTGTATCTC
TGTGCTTCCTCACTTGGGCAATTGAACACAGAGGCATTCTTTGGACAAGGCA
CCAGACTCACAGTTGTAGaagatctgaacaaggtgttccctccagaggtggccgtgttcgagccttctaaggccgag
atcgcccacacacaaaaagccaccctcgtgtgcctggccaccggctttttccccgaccacgtggaactgtcttggtgggtcaacggca
aagaggtgcactccggcgtgtcaacggatccccagcctctgaaagaacagcctgccctgaacgacagccggtactgcctgagctcc
agactgagagtgtccgccaccttctggcagaacccccggaaccacttcagatgccaggtgcagttttacggcctgagcgagaacgac
gagtggacccaggacagagccaagcccgtgacacaaatcgtgtctgccgaagcctggggaagagccgattgcggcatcaccagc
gcctcctatcaccagggcgtgctgagcgccacaatcctgtacgaaatcctgctgggcaaggccaccctgtacgccgtgctggtgtctg
ctctggtgctgatggccatggtcaagcggaaggactttggcagcggc (SEQ ID NO: 10)
Beta chain protein sequence
MGPRLLFWALLCLLGTGPVEAGVTQSPTHLIKTRGQQATLRCSPISGHTSVYWYQQ
ALGLGLQFLLWYDEGEERNRGNFPPRFSGRQFPNYSSELNVNALELEDSALYLCASS
LGQLNTEAFFGQGTRLTVVEdlnkvfppevavfepskaeiahtqkatlvclatgffpdhvelswwvngkevhs
gvstdpqplkeqpalndsryclssrlrvsatfwqnprnhfrcqvqfyglsendewtqdrakpvtqivsaeawgradcgitsasyhq
gvlsatilyeillgkatlyavlvsalvlmamvkrkdfgsg (SEQ ID NO: 11)
Complete Beta and Alpha ORF DNA Sequence (The underlined italic region in the “Furin-
P2A” site encodes a sequence allowing for expression of two polypeptide chains in a single
cassette)
ATGGGACCCAGGCTCCTCTTCTGGGCACTGCTTTGTCTCCTCGGAACAGGCCCAG
TGGAGGCTGGAGTCACACAAAGTCCCACACACCTGATCAAAACGAGAGGACAGC
AAGCGACTCTGAGATGCTCTCCTATCTCTGGGCACACCAGTGTGTACTGGTACC
AACAGGCCCTGGGTCTGGGCCTCCAGTTCCTCCTTTGGTATGACGAGGGTGAAG
AGAGAAACAGAGGAAACTTCCCTCCTAGATTTTCAGGTCGCCAGTTCCCTAATTA
TAGCTCTGAGCTGAATGTGAACGCCTTGGAGCTGGAGGACTCGGCCCTGTATCTC
TGTGCTTCCTCACTTGGGCAATTGAACACAGAGGCATTCTTTGGACAAGGCA
CCAGACTCACAGTTGTAGaagatctgaacaaggtgttccctccagaggtggccgtgttegagccttctaaggccgag
atcgcccacacacaaaaagccaccctegtgtgcctggccaccggctttttccccgaccacgtggaactgtcttggtgggtcaacggca
aagaggtgcactccggegtgtcaacggatccccagcctctgaaagaacagcetgccctgaacgacagceggtactgectgagctcc
agactgagagtgtccgccaccttctggcagaacccccggaaccacttcagatgccaggtgcagttttacggcctgagcgagaacgac
gagtggacccaggacagagccaagcccgtgacacaaatcgtgtctgccgaagcctggggaagagcegattgcggcatcaccagc
gcctcctatcaccagggcgtgctgagcgccacaatcctgtacgaaatcctgctgggcaaggccaccctgtacgccgtgctggtgtctg
ctctggtgctgatggccatggtcaagcggaaggactttggcagcggcagagccaaaaggtccgggagcgctGCGACAAAC
TTTAGCCTGTTGAAACAAGCCGGCGACGTTGAAGAGAACCCCGGACCTATGGAAAAA
ATGCTCGAGTGCGCCTTCATCGTGCTTTGGCTGCAGCTCGGATGGCTGAGCGGAG
AGGATCAAGTGACACAGTCTCCCGAGGCTCTGAGGCTGCAAGAGGGCGAAAGCA
GCTCCCTGAATTGCAGCTACACCGTGTCTGGCCTGAGGGGCCTGTTTTGGTACA
GACAAGACCCTGGCAAGGGACCCGAGTTCCTGTTCACACTGTACTCTGCCGGC
GAAGAAAAAGAGAAAGAGCGCCTGAAAGCAACCCTGACCAAGAAAGAGAGCTT
CCTGCACATCACAGCCCCTAAGCCAGAGGACAGCGCTACTTACCTGTGTGCCGT
TTCATACGGCCAGAATTTCGTTTTTGGTCCCGGAACCAGATTGTCCGTGCTGCC
CTacatccagaaccccgaccccgccgtgtaccagctgagggactccaagtccagcgacaagagcgtgtgtctgtttacggacttcg
acagccagaccaacgtgagtcaaagcaaggacagcgacgtctacataacggataagaccgtgctggacatgcggagcatggacttc
aagagcaacagcgccgtggcctggtccaacaagagcgacttcgcctgcgccaacgccttcaacaacagcatcatccccgaggaca
ccttcttccccagcagcgacgtgccctgcgacgtgaaactggtggagaagtccttcgagacagacaccaatctgaactttcagaacct
gctggtgatcgtgctgcggattctgctgctgaaagtggccggcttcaatctgctgatgaccctgcggctgtggagcagc (SEQ ID
NO: 12)
Complete Beta and Alpha ORF Protein Sequence (The underlined italic region in the “Furin-
P2A” site allows expression of two polypeptide chains in a single cassette)
MGPRLLFWALLCLLGTGPVEAGVTQSPTHLIKTRGQQATLRCSPISGHTSVYWYQQ
ALGLGLQFLLWYDEGEERNRGNFPPRFSGRQFPNYSSELNVNALELEDSALYLCASS
LGQLNTEAFFGQGTRLTVVEdlnkvfppevavfepskaeiahtqkatlvclatgffpdhvelswwvngkevhs
gvstdpqplkeqpalndsryclssrlrvsatfwqnprnhfrcqvqfyglsendewtqdrakpvtqivsaeawgradcgitsasyhq
gvlsatilyeillgkatlyavlvsalvlmamvkrkdfgsgrakrsgsgATNFSLLKQAGDVEENPGPMEKMLEC
AFIVLWLQLGWLSGEDQVTQSPEALRLQEGESSSLNCSYTVSGLRGLFWYRQDPGK
GPEFLFTLYSAGEEKEKERLKATLTKKESFLHITAPKPEDSATYLCAVSYGQNFVFG
PGTRLSVLPYiqnpdpavyqlrdskssdksvclftdfdsqtnvsqskdsdvyitdktvldmrsmdfksnsavawsnksdf
acanafnnsiipedtffpssdvpcdvklveksfetdtnlnfqnllvivlrilllkvagfnllmtlrlwss (SEQ ID NO: 13)
*Table 2 provides, in part, representative TCR sequences are grouped according to MHC serotype presentation and sub-grouped according to different peptides presented by the MHC serotype and bound by the sub-grouped TCRs. Individual TCRs, such as those representatively exemplified in the tables, are described and claimed, as well as the genus of binding proteins that bind a peptide epitope sequence described herein either alone or in a complex with an MHC, such as those grouped in the tables provided herein. In addition, TRAV, TRAJ, and TRAC genes for each TCR alpha chain described herein, and TRBV, TRBJ, and TRBC genes for each TCR beta chain described herein, are provided. Sequences for each TCR described herein are provided as pairs of cognate alpha chain and beta chains for each named TCR. TCR sequences described herein are annotated. Variable domain sequences are capitalized. Constant domain sequences are in lower case. CDR1, CDR2, and CDR3 sequences are annotated using bold and underlined text. CDR1, CDR2, and CDR3 are
shown in standard order of appearance from left (N-terminus) to right (C-terminus). TRAV, TRAJ, and TRAC genes for each TCR alpha chain described herein, and TRBV, TRBJ, and TRBC genes for each TCR beta chain described herein, are annotated according to well-known IMGT nomenclature described herein. Similarly, CDR1 and CDR2 of TRAV and TRBV are well-known in the art since they are based on well-known and annotated TRAV and TRBV sequences (e.g., as annotated in databases like IMGT available at imt.org and IEDB available at iedb.org).

TABLE 3
Representative Human MAGEA1 cDNA sequence
atgtctcttgagcagaggagtctgcactgcaagcctgaggaagcccttgaggcccaacaagaggccctgggcctggtgtgtgtgcag
gctgccacctcctcctcctctcctctggtcctgggcaccctggaggaggtgcccactgctgggtcaacagatcctccccagagtcctca
gggagcctccgcctttcccactaccatcaacttcactcgacagaggcaacccagtgagggttccagcagccgtgaagaggaggggc
caagcacctcttgtatcctggagtccttgttccgagcagtaatcactaagaaggtggctgatttggttggttttctgctcctcaaatatcgag
ccagggagccagtcacaaaggcagaaatgctggagagtgtcatcaaaaattacaagcactgttttcctgagatcttcggcaaagcctct
gagtccttgcagctggtctttggcattgacgtgaaggaagcagaccccaccggccactcctatgtccttgtcacctgcctaggtctctcc
tatgatggcctgctgggtgataatcagatcatgcccaagacaggcttcctgataattgtcctggtcatgattgcaatggagggcggccat
gctcctgaggaggaaatctgggaggagctgagtgtgatggaggtgtatgatgggagggagcacagtgcctatggggagcccagga
agctgctcacccaagatttggtgcaggaaaagtacctggagtaccggcaggtgccggacagtgatcccgcacgctatgagttcctgtg
gggtccaagggccctcgctgaaaccagctatgtgaaagtccttgagtatgtgatcaaggtcagtgcaagagttcgctttttcttcccat
ccctgcgtgaagcagctttgagagaggaggaagagggagtctga (SEQ ID NO: 14)
Representative Human MAGEA1 protein sequence
MSLEQRSLHCKPEEALEAQQEALGLVCVQAATSSSSPLVLGTLEEVPTAGSTDPPQSP
QGASAFPTTINFTRQRQPSEGSSSREEEGPSTSCILESLFRAVITKKVADLVGFLLLKYR
AREPVTKAEMLESVIKNYKHCFPEIFGKASESLQLVFGIDVKEADPTGHSYVLVTCLG
LSYDGLLGDNQIMPKTGFLIIVLVMIAMEGGHAPEEEIWEELSVMEVYDGREHSAYG
EPRKLLTQDLVQEKYLEYRQVPDSDPARYEFLWGPRALAETSYVKVLEYVIKVSAR
VRFFFPSLREAALREEEEGV* (SEQ ID NO: 15)
Representative Human HLA-A*02:01 DNA sequence
Atggccgtcatggcgccccgaaccctcgtcctgctactctcgggggctctggccctgacccagacctggggggctctcactccatg
aggtatttcttcacatccgtgtcccggcccggccgcggggagccccgcttcatcgcagtgggctacgtggacgacacgcagttcgtg
cggttcgacagcgacgccgcgagccagaggatggagccgcgggcgccgtggatagagcaggagggtccggagtattgggacgg
ggagacacggaaagtgaaggcccactcacagactcaccgagtggacctggggaccctgcgcggctactacaaccagagcgaggc
cggttctcacaccgtccagaggatgtatggctgcgacgtggggtcggactggcgcttcctccgcgggtaccaccagtacgcctacga
cggcaaggattacatcgccctgaaagaggacctgcgctcttggaccgcggcggacatggcagctcagaccaccaagcacaagtgg
gaggcggcccatgtggcggagcagttgagagcctacctggagggcacgtgcgtggagtggctccgcagatacctggagaacggg
aaggagacgctgcagcgcacggacgcccccaaaacgcatatgactcaccacgctgtctctgaccatgaagccaccctgaggtgctg
ggccctgagcttctaccctgcggagatcacactgacctggcagcgggatggggaggaccagacccaggacacggagctcgtgga
gaccaggcctgcaggggatggaaccttccagaagtgggggctgtggtggtgccttctggacaggagcagagatacacctgccatg
tgcagcatgagggtttgcccaagcccctcaccctgagatgggagccgtcttcccagcccaccatccccatcgtgggcatcattgctgg
cctggttctctttggagctgtgatcactggagctgtggtcgctgctgtgatgtggaggaggaagagctcagatagaaaaggagggagc
tactctcaggctgcaagcagtgacagtgcccagggctctgatgtgtctctcacagcttgtaaagtgtga (SEQ ID NO: 16)
Representative Human HLA-A*02:01 protein sequence
MAVMAPRTLVLLLSGALALTQTWAGSHSMRYFFTSVSRPGRGEPRFIAVGYVDDTQ
FVRFDSDAASQRMEPRAPWIEQEGPEYWDGETRKVKAHSQTHRVDLGTLRGYYNQ
SEAGSHTVQRMYGCDVGSDWRFLRGYHQYAYDGKDYIALKEDLRSWTAADMAAQ
TTKHKWEAAHVAEQLRAYLEGTCVEWLRRYLENGKETLQRTDAPKTHMTHHAVS
DHEATLRCWALSFYPAEITLTWQRDGEDQTQDTELVETRPAGDGTFQKWAAVVVPS
GQEQRYTCHVQHEGLPKPLTLRWEPSSQPTIPIVGIIAGLVLFGAVITGAVVAAVMW
RRKSSDRKGGSYSQAASSDSAQGSDVSLTACKV* (SEQ ID NO: 17)
Representative Vector (the TCR-encoding protein of which can be interchanged with
any TCR sequence of interest): pTSLV102-MSCV-HA1-10-30-MGTM-Q-CD8
tggaagggctaattcactcccaaagaagacaagatatccttgatctgtggatctaccacacacaaggctacttccctgattagcagaact
acacaccagggccaggggtcagatatccactgacctttggatggtgctacaagctagtaccagttgagccagataaggtagaagagg
ccaataaaggagagaacaccagcttgttacaccctgtgagcctgcatgggatggatgacccggagagagaagtgttagagtggaggt
ttgacagccgcctagcatttcatcacgtggcccgagagctgcatccggagtacttcaagaactgctgatatcgagcttgctacaaggga
ctttccgctggggactttccagggaggcgtggcctggggggactggggagtggcgagccctcagatcctgcatataagcagctgct
ttttgcctgtactgggtctctctggttagaccagatctgagcctgggagctctctggctaactagggaacccactgcttaagcctcaataa
agcttgccttgagtgcttcaagtagtgtgtgcccgtctgttgtgtgactctggtaactagagatccctcagacccttttagtcagtgtggaa
aatctctagcagtggcgcccgaacagggacttgaaagcgaaagggaaaccagaggagctctctcgacgcaggactcggcttgctga
agcgcgcacggcaagaggcgaggggcggcgactggtgagtacgccaaaaattttgactagcggaggctagaaggagagagatgg
gtgcgagagcgtcagtattaagcgggggagaattagatcgcgatgggaaaaaattcggttaaggccagggggaaagaaaaaatata
aattaaaacatatagtatgggcaagcagggagctagaacgattcgcagttaatcctggcctgttagaaacatcagaaggctgtagacaa
atactgggacagctacaaccatcccttcagacaggatcagaagaacttagatcattatataatacagtagcaaccctctattgtgtgcatc
aaaggatagagataaaagacaccaaggaagctttagacaagatagaggaagagcaaaacaaaagtaagaccaccgcacagcaag
cggccggccgctgatcttcagacctggaggaggagatatgagggacaattggagaagtgaattatataaatataaagtagtaaaaatt
gaaccattaggagtagcacccaccaaggcaaagagaagagtggtgcagagagaaaaaagagcagtgggaataggagctttgttcct
tgggttcttgggagcagcaggaagcactatgggcgcagcgtcaatgacgctgacggtacaggccagacaattattgtctggtatagtg
cagcagcagaacaatttgctgagggctattgaggcgcaacagcatctgttgcaactcacagtctggggcatcaagcagctccaggca
agaatcctggctgtggaaagatacctaaaggatcaacagctcctggggatttggggttgctctggaaaactcatttgcaccactgctgtg
ccttggaatgctagttggagtaataaatctctggaacagatttggaatcacacgacctggatggagtgggacagagaaattaacaattac
acaagcttaatacactccttaattgaagaatcgcaaaaccagcaagaaaagaatgaacaagaattattggaattagataaatgggcaag
tttgtggaattggtttaacataacaaattggctgtggtatataaaattattcataatgatagtaggaggcttggtaggtttaagaatagtttttg
ctgtactttctatagtgaatagagttaggcagggatattcaccattatcgtttcagacccacctcccaaccccgaggggacccgacaggc
ccgaaggaatagaagaagaaggtggagagagagacagagacagatccattcgattagtgaacggatctcgacggtatcgccgaatt
aattcacaaatggcagtattcatccacaattttaaaagaaaaggggggattggggggtacagtgcaggggaaagaatagtagacataa
tagcaacagacatacaaactaaagaattacaaaaacaaattacaaaaattcaaaattttcgggtttattacaggCGcGCcagagatcc
agtttggacCTgcAGGTGAAAGACCCCACCTGTAGGTTTGGCAAGTAGCTTAAGTA
ACGCCATTTTGCAAGGCATGGAAAATACATAACTGAGAATAGAGAAGTTCAG
ATCAAGGTTAGGAACAGAGAGACAGCAGAATATGGGCCAAACAGGATATCT
GTGGTAAGCAGTTCCTGCCCCGGCTCAGGGCCAAGAACAGATGGTCCCCAG
ATGCGGTCCCGCCCTCAGCAGTTTCTAGCGAACCATCAGATGTTTCCAGGGT
GCCCCAAGGACCTGAAATGACCCTGTGCCTTATTTGAACTAACCAATCAGTT
tGCTTCTtGCTTCTGTTtGtGtGCTTCTGCTCCCtGAGCTCAATAAAAGAGCCCA
CAACCCCTCACTtGGtGgGCCAGTCCTCtGATAGACTGtGTCcCCtGGaTACCCGTAc
ggtaccgctagcgccaccATGGGCACCAGCCTCCTCTGCTGGATGGCCCTGTGTCTCCTGGG
GGCAGATCACGCAGATACTGGAGTCTCCCAGGACCCCAGACACAAGATCACAAAG
AGGGGACAGAATGTTACTTTCAGGTGTGATCCAATTTCTGAACACAACCGCCTTTA
TTGGTACCGCCAGACCCTGGGGCAGGGCCCAGAGTTTCTGACTTACTTCCAGAATG
AAGCTCAACTTGAAAAATCAAGGCTGCTCAGTGATCGGTTCTCTGCAGAGAGGCCT
AAGGGATCTTTCTCCACCTTGGAGATCCAGCGCACAGAGCAGGGGGACTCTGCCAT
GTATCTCTGTGCCAGCAGCCGCACTGCTGGAGATACTCAGTATTTTGGCCCAGGCA
CCCGGCTGACAGTGCTCGAAGATCTGAACAAGGTGTTCCCTCCAGAGGTGGCCGT
GTTCGAGCCTTCTaAGGCCGAGATCgccCACACaCAaAAAGCCACCCTCGTGTGCCT
GGCCACCGGCTTTTTCCCCGACCACGTGGAACTGTCTTGGTGGGTCAACGGCAAAG
AGGTGCACTCCGGCGTGtcAACgGATCCCCAGCCTCTGAAAGAACAGCCTGCCCTGA
ACGACAGCCGGTACTGCCTGAGCTCCAGACTGAGAGTGTCCGCCACCTTCTGGCA
GAACCCCCGGAACCACTTCAGATGCCAGGTGCAGTTTTACGGCCTGAGCGAGAAC
GACGAGTGGACCCAGGACAGAGCCAAGCCCGTGACACAAATCGTGTCTGCCGAAG
CCTGGGGAAGAGCCGATTGCGGCATCACCAGCGCCTCCTATCACCAGGGCGTGCT
GAGCGCCACAATCCTGTACGAAATCCTGCTGGGCAAGGCCACCCTGTACGCCGTG
CTGGTGTCTGCTCTGGTGCTGATGGCCATGGTCAAGCGGAAGGACTTTGGCAGCG
GCAGAGCCAAAAGGTCCGGGAGCGGTGCGACAAACTTTAGCCTGTTGAAACAAG
CCGGCGACGTTGAAGAGAACCCCGGACCTATGGAAACCCTCTTGGGCCTGCTT
ATCCTTTGGCTGCAGCTGCAATGGGTGAGCAGCAAACAGGAGGTGACTCAG
ATTCCTGCAGCTCTGAGTGTCCCAGAAGGAGAAAACTTGGTTCTCAACTGCA
GTTTCACTGATAGCGCTATTTACAACCTCCAGTGGTTTAGGCAGGACCCTGG
GAAAGGCCTCACATCTCTGTTGCTTATTCAGTCAAGTCAGAGAGAGCAAACA
AGTGGACGCCTTAATGCCTCTCTGGATAAATCATCAGGACGCAGTACTCTTT
ACATTGCAGCTTCTCAGCCTGGTGATTCAGCCACCTACCTGTGCGCTGTGAG
GGGTGGTACCTCAGGAACCTACAAATACATCTTTGGAACAGGCACCAGGCT
GAAGGTTCTTGCAAACATCCAGAACCCCGACCCCGCCGTGTACCAGCTGAG
GGACTCCAAGTCCAGCGACAAGAGCGTGTGTCTGTTTACGGACTTCGACAG
CCAGACCAACGTGAGTCAAAGCAAGGACAGCGACGTCTACATAACGGATAA
GACCGTGCTGGACATGCGGAGCATGGACTTCAAGAGCAACAGCGCCGTGGC
CTGGTCCAACAAGAGCGACTTCGCCTGCGCCAACGCCTTCAACAACAGCAT
CATCCCCGAGGACACCTTCTTCCCCAGCAGCGACGTGCCCTGCGACGTGAA
ACTGGTGGAGAAGTCCTTCGAGACAGACACCAATCTGAACTTTCAGAACCTG
CTGGTGATCGTGCTGCGGATTCTGCTGCTGAAAGTGGCCGGCTTCAATCTG
CTGATGACCCTGCGGCTGTGGAGCAGCAGGGCTAAGAGGTCCGGCAGCGGAG
CCACCAATTTTTCCCTGCTGAAACAGGCTGGTGACGTGGAAGAAAACCCTGGCCC
CATGGCGCTGCCCGTCACCGCGCTGCTGCTGCCCCTGGCGCTGCTGTTACACGCC
GCTCGGCCAGAGCTTCCCACCCAGGGCACATTCTCCAACGTGTCCACCAATGTGTCG
GGAGGCGGCGGATCGTCCCAGTTCAGAGTGTCCCCTCTGGACCGCACCTGGAACCTG
GGCGAGACCGTGGAGCTGAAATGTCAGGTCCTGCTGAGCAACCCGACCTCCGGGTGC
AGTTGGCTGTTCCAGCCGCGTGGTGCTGCCGCAAGCCCTACGTTCCTGCTTTACCTGA
GCCAGAACAAGCCCAAGGCGGCCGAGGGCCTGGACACCCAGAGATTCTCCGGCAAG
CGCCTGGGGGACACATTCGTGCTTACTTTGAGCGATTTCCGCAGAGAGAACGAGGGCT
ACTATTTCTGTTCGGCGCTGAGCAATTCCATCATGTATTTCAGCCACTTTGTGCCAGTG
TTCCTGCCTGCCAAGCCTACCACAACACCAGCTCCCCGTCCCCCGACTCCGGCGCCT
ACCATCGCGAGTCAACCGTTGAGCCTGAGGCCTGAGGCTTGTCGGCCCGCTGCGGGG
GGTGCCGTCCACACCAGGGGCCTCGACTTTGCGTGCGACATCTATATTTGGGCGCCT
CTGGCGGGTACCTGCGGGGTGCTGCTGCTGTCATTGGTGATTACCCTGTACTGCAATC
ACCGCAACCGCCGGCGGGTCTGTAAGTGCCCACGGCCTGTGGTCAAGTCCGGTGACA
AACCGTCGCTCTCGGCTCGCTACGTGCGCGCTAAGCGCAGCGGTTCCGGGGCCACC
AACTTTTCATTGCTGAAGCAGGCCGGTGATGTGGAGGAGAATCCAGGGCCCATG
CGCCCCAGGCTTTGGCTCCTTCTTGCTGCTCAGCTCACTGTCTTGCATGGCAACTC
CGTTCTGCAGCAGACTCCCGCCTACATCAAGGTGCAGACGAACAAGATGGTGAT
GCTGTCATGCGAGGCCAAGATCTCTCTTTCAAATATGAGAATTTATTGGCTACGA
CAGCGCCAGGCCCCCTCCAGCGACAGCCACCACGAGTTCCTGGCGCTTTGGGATT
CTGCTAAAGGCACCATCCATGGAGAGGAGGTGGAACAGGAGAAGATAGCTGTCT
TCCGCGACGCATCCCGCTTCATCCTGAACCTGACCAGCGTGAAGCCGGAGGACA
GCGGCATCTACTTCTGTATGATCGTTGGCTCCCCCGAGCTGACCTTCGGCAAAGG
CACCCAGCTGTCCGTGGTGGACTTCCTGCCCACCACAGCCCAGCCAACCAAGAA
ATCCACCCTCAAGAAGCGCGTGTGCCGACTGCCCCGCCCTGAAACCCAGAAGGG
CCCTCTGTGCTCCCCCATCACCCTTGGACTGCTGGTGGCGGGAGTCCTGGTGCTG
CTCGTATCTCTGGGTGTCGCCATCCACCTGTGCTGCCGCCGCCGCCGCGCCCGCC
TGAGGTTTATGAAACAGTTTTACAAGTGATAAatcgatagatcctaatcaacctctggattacaaaatttg
tgaaagattgactggtattcttaactatgttgctccttttacgctatgtggatacgctgctttaatgcctttgtatcatgctattgcttcccgta
tggctttcattttctcctccttgtataaatcctggttgctgtctctttatgaggagttgtggcccgttgtcaggcaacgtggcgtggtgtgcact
gtgtttgctgacgcaacccccactggttggggcattgccaccacctgtcagctcctttccgggactttcgctttccccctccctattgccacg
gcggaactcatcgccgcctgccttgcccgctgctggacaggggctcggctgttgggcactgacaattccgtggtgttgtcggggaaat
catcgtcctttccttggctgctcgcctgtgttgccacctggattctgcgcgggacgtccttctgctacgtcccttcggccctcaatccagcg
gaccttccttcccgcggcctgctgccggctctgcggcctcttccgcgtcttcgccttcgccctcagacgagtcggatctccctttgggcc
gcctccccgcctgagatcctttaagaccaatgacttacaaggcagctgtagatcttagccactttttaaaagaaaaggggggactggaa
gggctaattcactcccaacgaagacaagatctgctttttgcttgtactgggtctctctggttagaccagatctgagcctgggagctctctg
gctaactagggaacccactgcttaagcctcaataaagcttgccttgagtgcttcaagtagtgtgtgcccgtctgttgtgtgactctggtaa
ctagagatccctcagacccttttagtcagtgtggaaaatctctagcagtagtagttcatgtcatcttattattcagtatttataacttgcaaaga
aatgaatatcagagagtgagaggcccgggttaattaaggaaagggctagatcattcttgaagacgaaagggcctcgtgatacgcctat
ttttataggttaatgtcatgataataatggtttcttagacgtcaggtggcacttttcggggaaatgtgcgcggaacccctatttgtttatttttc
taaatacattcaaatatgtatccgctcatgagacaataaccctgataaatgcttcaataatattgaaaaaggaagagtatgagtattcaacat
ttccgtgtcgcccttattcccttttttgcggcattttgccttcctgtttttgctcacccagaaacgctggtgaaagtaaaagatgctgaagatc
agttgggtgcacgagtgggttacatcgaactggatctcaacagcggtaagatccttgagagttttcgccccgaagaacgttttccaatga
tgagcacttttaaagttctgctatgtggcgcggtattatcccgtgttgacgccgggcaagagcaactcggtcgccgcatacactattctca
gaatgacttggttgagtactcaccagtcacagaaaagcatcttacggatggcatgacagtaagagaattatgcagtgctgccataacca
tgagtgataacactgcggccaacttacttctgacaacgatcggaggaccgaaggagctaaccgcttttttgcacaacatgggggatcat
gtaactcgccttgatcgttgggaaccggagctgaatgaagccataccaaacgacgagcgtgacaccacgatgcctgtagcaatggca
acaacgttgcgcaaactattaactggcgaactacttactctagcttcccggcaacaattaatagactggatggaggcggataaagttgca
ggaccacttctgcgctcggcccttccggctggctggtttattgctgataaatctggagccggtgagcgtgggtctcgcggtatcattgca
gcactggggccagatggtaagccctcccgtatcgtagttatctacacgacggggagtcaggcaactatggatgaacgaaatagacag
atcgctgagataggtgcctcactgattaagcattggtaactgtcagaccaagtttactcatatatactttagattgatttaaaacttcattttta
atttaaaaggatctaggtgaagatcctttttgataatctcatgaccaaaatcccttaacgtgagttttcgttccactgagcgtcagaccccgt
agaaaagatcaaaggatcttcttgagatcctttttttctgcgcgtaatctgctgcttgcaaacaaaaaaaccaccgctaccagcggtggttt
gtttgccggatcaagagctaccaactctttttccgaaggtaactggcttcagcagagcgcagataccaaatactgttcttctagtgtagcc
gtagttaggccaccacttcaagaactctgtagcaccgcctacatacctcgctctgctaatcctgttaccagtggctgctgccagtggcga
taagtcgtgtcttaccgggttggactcaagacgatagttaccggataaggcgcagcggtcgggctgaacggggggttcgtgcacaca
gcccagcttggagcgaacgacctacaccgaactgagatacctacagcgtgagctatgagaaagcgccacgcttcccgaagggaga
aaggcggacaggtatccggtaagcggcagggtcggaacaggagagcgcacgagggagcttccagggggaaacgcctggtatctt
tatagtcctgtcgggtttcgccacctctgacttgagcgtcgatttttgtgatgctcgtcaggggggcggagcctatggaaaaacgccagc
aacgcggcctttttacggttcctggccttttgctggccttttgctcacatgttctttcctgcgttatccCCTGATTCTGTGGATA
ACCGTATTACCGCCTTTGAGTGAGCTGATACCGCTCGCCGCAGCCGAACGACCGA
GCGCAGCGAGTCAGTGAGCGAGGAAGCGGAAGAGCGCCCAATACGCAAACCGC
CTCTCCCCGCGCGTTGGCCGATTCATTAATGCAGCAAGCTCATGGCTGACTAATT
TTTTTTATTTATGCAGAGGCCGAGGCCGCCTCGGCCTCTGAGCTATTCCAGAAGT
AGTGAGGAGGCTTTTTTGGAGGCCTAGGCTTTTGCAAAAAGCTCCCCGTGGCACG
ACAGGTTTCCCGACTGGAAAGCGGGCAGTGAGCGCAACGCAATTAATGTGAGTT
AGCTCACTCATTAGGCACCCCAGGCTTTACACTTTATGCTTCCGGCTCGTATGTTG
TGTGGAATTGTGAGCGGATAACAATTTCACACAGGAAACAGCTATGACATGATT
ACGAATTTCACAAATAAAGCATTTTTTTCACTGCATTCTAGTTGTGGTTTGTCCAA
ACTCATCAATGTATCTTATCATGTCTGGATCAACTGGATAACTCAAGCTAACCAA
AATCATCCCAAACTTCCCACCCCATACCCTATTACCACTGCCAATTACCTGTGGTT
TCATTTACTCTAAACCTGTGATTCCTCTGAATTATTTTCATTTTAAAGAAATTGTA
TTTGTTAAATATGTACTACAAACTtagtagt (SEQ ID NO: 18)
Representative Vector (the TCR-encoding protein of which can be interchanged with
any TCR sequence of interest): pHAGE-MSCV-HN-P32-41-P2A-dnTGFbRII (with
dnTGFbRII highlighted in bold text)
tggaagggctaattcactcccaaagaagacaagatatccttgatctgtggatctaccacacacaaggctacttccctgattagcagaact
acacaccagggccaggggtcagatatccactgacctttggatggtgctacaagctagtaccagttgagccagataaggtagaagagg
ccaataaaggagagaacaccagcttgttacaccctgtgagcctgcatgggatggatgacccggagagagaagtgttagagtggaggt
ttgacagccgcctagcatttcatcacgtggcccgagagctgcatccggagtacttcaagaactgctgatatcgagcttgctacaaggga
ctttccgctggggactttccagggaggcgtggcctgggcgggactggggagtggcgagccctcagatcctgcatataagcagctgct
ttttgcctgtactgggtctctctggttagaccagatctgagcctgggagctctctggctaactagggaacccactgcttaagcctcaataa
agcttgccttgagtgcttcaagtagtgtgtgcccgtctgttgtgtgactctggtaactagagatccctcagacccttttagtcagtgtggaa
aatctctagcagtggcgcccgaacagggacttgaaagcgaaagggaaaccagaggagctctctcgacgcaggactcggcttgctga
agcgcgcacggcaagaggcgaggggcggcgactggtgagtacgccaaaaattttgactagcggaggctagaaggagagagatgg
gtgcgagagcgtcagtattaagcgggggagaattagatcgcgatgggaaaaaattcggttaaggccagggggaaagaaaaaatata
aattaaaacatatagtatgggcaagcagggagctagaacgattcgcagttaatcctggcctgttagaaacatcagaaggctgtagacaa
atactgggacagctacaaccatcccttcagacaggatcagaagaacttagatcattatataatacagtagcaaccctctattgtgtgcatc
aaaggatagagataaaagacaccaaggaagctttagacaagatagaggaagagcaaaacaaaagtaagaccaccgcacagcaag
cggccggccgctgatcttcagacctggaggaggagatatgagggacaattggagaagtgaattatataaatataaagtagtaaaaatt
gaaccattaggagtagcacccaccaaggcaaagagaagagtggtgcagagagaaaaaagagcagtgggaataggagctttgttcct
tgggttcttgggagcagcaggaagcactatgggcgcagcgtcaatgacgctgacggtacaggccagacaattattgtctggtatagtg
cagcagcagaacaatttgctgagggctattgaggcgcaacagcatctgttgcaactcacagtctggggcatcaagcagctccaggca
agaatcctggctgtggaaagatacctaaaggatcaacagctcctggggatttggggttgctctggaaaactcatttgcaccactgctgtg
ccttggaatgctagttggagtaataaatctctggaacagatttggaatcacacgacctggatggagtgggacagagaaattaacaattac
acaagcttaatacactccttaattgaagaatcgcaaaaccagcaagaaaagaatgaacaagaattattggaattagataaatgggcaag
tttgtggaattggtttaacataacaaattggctgtggtatataaaattattcataatgatagtaggaggcttggtaggtttaagaatagtttttg
ctgtactttctatagtgaatagagttaggcagggatattcaccattatcgtttcagacccacctcccaaccccgaggggacccgacaggc
ccgaaggaatagaagaagaaggtggagagagagacagagacagatccattcgattagtgaacggatctcgacggtatcgccgaatt
aattcacaaatggcagtattcatccacaattttaaaagaaaaggggggattggggggtacagtgcaggggaaagaatagtagacataa
tagcaacagacatacaaactaaagaattacaaaaacaaattacaaaaattcaaaattttcgggtttattacaggCGcGCcagagatcc
agtttggacCTgcAGGTGAAAGACCCCACCTGTAGGTTTGGCAAGtTAGCTTAAGTAAC
GCCATTTTGCAAGGCATGGAAAATACATAACTGAGAATAGAGAAGTTCAGATCA
AGGTTAGGAACAGAGAGACAGCAGAATATGGGCCAAACAGGATATCTGTGGTAA
GCAGTTCCTGCCCCGGCTCAGGGCCAAGAACAGATGGTCCCCAGATGCGGTCCC
GCCCTCAGCAGTTTCTAGCGAACCATCAGATGTTTCCAGGGTGCCCCAAGGACCT
GAAATGACCCTGTGCCTTATTTGAACTAACCAATCAGTTtGCTTCTtGCTTCTGTTtG
tGtGCTTCTGCTCCCtGAGCTCAATAAAAGAGCCCACAACCCCTCACTtGGtGgGCCA
GTCCTCtGATAGACTGtGTCcCCtGGaTACCCGTAcggtaccgctagegccaccATGGGCTCCT
GGACCCTCTGCTGTGTGTCCCTTTGCATCCTGGTTGCAAAGCACACAGATGCTGG
AGTTATCCAGTCACCCCGGCACGAGGTGACAGAGATGGGACAAGAAGTGACTCT
GAGATGTAAACCAATTTCAGGACATGACTACCTTTTCTGGTACAGACAGACCATG
ATGCGGGGACTGGAGTTGCTCATTTACTTTAACAACAACGTTCCTATTGATGATT
CAGGGATGCCCGAGGATCGCTTCTCAGCTAAGATGCCTAATGCATCATTCTCCAC
TCTGAAGATCCAGCCCTCAGAACCCAGGGACTCAGCTGTGTACTTCTGTGCCAGC
AGTTTTCTCGGCTGGAATGAAAAACTGTTCTTTGGCAGTGGAACCCAGCTCTCTG
TCTTGGAAGATCTGAACAAGGTGTTCCCTCCAGAGGTGGCCGTGTTCGAGCCTTC
TaAGGCCGAGATCgccCACACaCAaAAAGCCACCCTCGTGTGCCTGGCCACCGGCTT
TTTCCCCGACCACGTGGAACTGTCTTGGTGGGTCAACGGCAAAGAGGTGCACTCC
GGCGTGtcAACgGATCCCCAGCCTCTGAAAGAACAGCCTGCCCTGAACGACAGCC
GGTACTGCCTGAGCTCCAGACTGAGAGTGTCCGCCACCTTCTGGCAGAACCCCCG
GAACCACTTCAGATGCCAGGTGCAGTTTTACGGCCTGAGCGAGAACGACGAGTG
GACCCAGGACAGAGCCAAGCCCGTGACACAAATCGTGTCTGCCGAAGCCTGGGG
AAGAGCCGATTGCGGCATCACCAGCGCCTCCTATCACCAGGGCGTGCTGAGCGC
CACAATCCTGTACGAAATCCTGCTGGGCAAGGCCACCCTGTACGCCGTGCTGGTG
TCTGCTCTGGTGCTGATGGCCATGGTCAAGCGGAAGGACTTTGGCAGCGGCAGA
GCCAAAAGGTCCGGGAGCGGTGCGACAAACTTTAGCCTGTTGAAACAAGCCGGC
GACGTTGAAGAGAACCCCGGACCTATGGTCCTGAAATTCTCCGTGTCCATTCTTT
GGATTCAGTTGGCATGGGTGAGCACCCAGCTGCTGGAGCAGAGCCCTCAGTTTCT
TAGCATCCAAGAGGGAGAAAATCTCACTGTGTACTGCAACTCCTCAAGTGTTTTC
TCCAGCCTTCAATGGTACAGACAGGAGCCTGGGGAAGGTCCTGTCCTCCTGGTGA
CAGTTGTTACTGGTGGAGAAGTGAAGAAGCTGAAGAGACTTACCTTTCAGTTTGG
TGATGCAAGAAAGGACAGTTCTCTCCACATCACTGCAGCCCAGCCTGGTGATACA
GGCCTCTACCTCTGTGCAGGAGATGAAAGTATTAGCTATGGAAAGCTGACATTTG
GACAAGGGACCATCTTGACTGTCCATCCAAacatccagaaccccgaccccgccgtgtaccagctgagg
gactccaagtccagcgacaagagcgtgtgtctgtttacggacttcgacagccagaccaacgtgagtcaaagcaaggacagcgacgt
ctacataacggataagaccgtgctggacatgcggagcatggacttcaagagcaacagcgccgtggcctggtccaacaagagcgact
tcgcctgcgccaacgccttcaacaacagcatcatccccgaggacaccttcttccccagcagcgacgtgccctgcgacgtgaaactgg
tggagaagtccttcgagacagacaccaatctgaactttcagaacctgctggtgatcgtgctgcggattctgctgCTGAAAGTG
GCCGGCTTCAATCTGCTGATGACCCTGCGGCTGTGGAGCAGCAGGGCTAAGAGG
TCCGGCAGCGGAGCCACCAATTTTTCCCTGCTGAAACAGGCTGGTGACGTGGAA
GAAAACCCTGGCCCCATGGGTCGGGGGCTGCTCAGGGGCCTGTGGCCGCTG
CACATCGTCCTGTGGACGCGTATCGCCAGCACGATCCCACCGCACGTTCAG
AAGTCGGTTAATAACGACATGATAGTCACTGACAACAACGGTGCAGTCAAGT
TTCCACAACTGTGTAAATTTTGTGATGTGAGATTTTCCACCTGTGACAACCA
GAAATCCTGCATGAGCAACTGCAGCATCACCTCCATCTGTGAGAAGCCACA
GGAAGTCTGTGTGGCTGTATGGAGAAAGAATGACGAGAACATAACACTAGA
GACAGTTTGCCATGACCCCAAGCTCCCCTACCATGACTTTATTCTGGAAGAT
GCTGCTTCTCCAAAGTGCATTATGAAGGAAAAAAAAAAGCCTGGTGAGACTT
TCTTCATGTGTTCCTGTAGCTCTGATGAGTGCAATGACAACATCATCTTCTC
AGAAGAATATAACACCAGCAATCCTGACTTGTTGCTAGTCATATTTCAAGTG
ACAGGCATCAGCCTCCTGCCACCACTGGGAGTTGCCATATCTGTCATCATCA
TCTTCTACTGCTACCGCGTTaaccggcagcagaagTAGTGATAAatcgatagatcctaatcaacctct
ggattacaaaatttgtgaaagattgactggtattcttaactatgttgctccttttacgctatgtggatacgctgctttaatgcctttgtatcatg
ctattgcttcccgtatggctttcattttctcctccttgtataaatcctggttgctgtctctttatgaggagttgtggcccgttgtcaggcaacgt
ggcgtggtgtgcactgtgtttgctgacgcaacccccactggttggggcattgccaccacctgtcagctcctttccgggactttcgctttcccc
ctccctattgccacggcggaactcatcgccgcctgccttgcccgctgctggacaggggctcggctgttgggcactgacaattccgtgg
tgttgtcggggaaatcatcgtcctttccttggctgctcgcctgtgttgccacctggattctgcgcgggacgtccttctgctacgtcccttcg
gccctcaatccagcggaccttccttcccgcggcctgctgccggctctgcggcctcttccgcgtcttcgccttcgccctcagacgagtcg
gatctccctttgggccgcctccccgcctgagatcctttaagaccaatgacttacaaggcagctgtagatcttagccactttttaaaagaaa
aggggggactggaagggctaattcactcccaacgaagacaagatctgctttttgcttgtactgggtctctctggttagaccagatctgag
cctgggagctctctggctaactagggaacccactgcttaagcctcaataaagcttgccttgagtgcttcaagtagtgtgtgcccgtctgtt
gtgtgactctggtaactagagatccctcagacccttttagtcagtgtggaaaatctctagcagtagtagttcatgtcatcttattattcagtatt
tataacttgcaaagaaatgaatatcagagagtgagaggcccgggttaattaaggaaagggctagatcattcttgaagacgaaagggcc
tcgtgatacgcctatttttataggttaatgtcatgataataatggtttcttagacgtcaggtggcacttttcggggaaatgtgcgcggaaccc
ctatttgtttatttttctaaatacattcaaatatgtatccgctcatgagacaataaccctgataaatgcttcaataatattgaaaaaggaagagt
atgagtattcaacatttccgtgtcgcccttattcccttttttgcggcattttgccttcctgtttttgctcacccagaaacgctggtgaaagtaaa
agatgctgaagatcagttgggtgcacgagtgggttacatcgaactggatctcaacagcggtaagatccttgagagttttcgccccgaag
aacgttttccaatgatgagcacttttaaagttctgctatgtggcgcggtattatcccgtgttgacgccgggcaagagcaactcggtcgcc
gcatacactattctcagaatgacttggttgagtactcaccagtcacagaaaagcatcttacggatggcatgacagtaagagaattatgca
gtgctgccataaccatgagtgataacactgcggccaacttacttctgacaacgatcggaggaccgaaggagctaaccgcttttttgcac
aacatgggggatcatgtaactcgccttgatcgttgggaaccggagctgaatgaagccataccaaacgacgagcgtgacaccacgatg
cctgtagcaatggcaacaacgttgcgcaaactattaactggcgaactacttactctagcttcccggcaacaattaatagactggatggag
gcggataaagttgcaggaccacttctgcgctcggcccttccggctggctggtttattgctgataaatctggagccggtgagcgtgggtc
tcgcggtatcattgcagcactggggccagatggtaagccctcccgtatcgtagttatctacacgacggggagtcaggcaactatggat
gaacgaaatagacagatcgctgagataggtgcctcactgattaagcattggtaactgtcagaccaagtttactcatatatactttagattga
tttaaaacttcatttttaatttaaaaggatctaggtgaagatcctttttgataatctcatgaccaaaatcccttaacgtgagttttcgttccact
gagcgtcagaccccgtagaaaagatcaaaggatcttcttgagatcctttttttctgcgcgtaatctgctgcttgcaaacaaaaaaaccaccg
ctaccagcggtggtttgtttgccggatcaagagctaccaactctttttccgaaggtaactggcttcagcagagcgcagataccaaatact
gttcttctagtgtagccgtagttaggccaccacttcaagaactctgtagcaccgcctacatacctcgctctgctaatcctgttaccagtggc
tgctgccagtggcgataagtcgtgtcttaccgggttggactcaagacgatagttaccggataaggcgcagcggtcgggctgaacggg
gggttcgtgcacacagcccagcttggagcgaacgacctacaccgaactgagatacctacagcgtgagctatgagaaagcgccacgc
ttcccgaagggagaaaggcggacaggtatccggtaagcggcagggtcggaacaggagagcgcacgagggagcttccaggggga
aacgcctggtatctttatagtcctgtcgggtttcgccacctctgacttgagcgtcgatttttgtgatgctcgtcaggggggcggagcctatg
gaaaaacgccagcaacgcggcctttttacggttcctggccttttgctggccttttgctcacatgttctttcctgcgttatccCCTGATT
CTGTGGATAACCGTATTACCGCCTTTGAGTGAGCTGATACCGCTCGCCGCAGCCG
AACGACCGAGCGCAGCGAGTCAGTGAGCGAGGAAGCGGAAGAGCGCCCAATAC
GCAAACCGCCTCTCCCCGCGCGTTGGCCGATTCATTAATGCAGCAAGCTCATGGC
TGACTAATTTTTTTTATTTATGCAGAGGCCGAGGCCGCCTCGGCCTCTGAGCTATT
CCAGAAGTAGTGAGGAGGCTTTTTTGGAGGCCTAGGCTTTTGCAAAAAGCTCCCC
GTGGCACGACAGGTTTCCCGACTGGAAAGCGGGCAGTGAGCGCAACGCAATTAA
TGTGAGTTAGCTCACTCATTAGGCACCCCAGGCTTTACACTTTATGCTTCCGGCTC
GTATGTTGTGTGGAATTGTGAGCGGATAACAATTTCACACAGGAAACAGCTATG
ACATGATTACGAATTTCACAAATAAAGCATTTTTTTCACTGCATTCTAGTTGTGGT
TTGTCCAAACTCATCAATGTATCTTATCATGTCTGGATCAACTGGATAACTCAAG
CTAACCAAAATCATCCCAAACTTCCCACCCCATACCCTATTACCACTGCCAATTA
CCTGTGGTTTCATTTACTCTAAACCTGTGATTCCTCTGAATTATTTTCATTTTAAA
GAAATTGTATTTGTTAAATATGTACTACAAACTtagtagt (SEQ ID NO: 19)
Representative Vector (the TCR-encoding protein of which can be interchanged with
any TCR sequence of interest): pNVVD136_TSC-204-A02_TCR-1479_MSCV-TCR-
1479-CD8-EF1α-dnTGFbRII-DHFR
GCTAGCTGGCTTGTTGTCCACAACCATTAAACCTTAAAAGCTTTAAAAGCCTTAT
ATATTCTTTTTTTTCTTATAAAACTTAAAACCTTAGAGGCTATTTAAGTTGCTGAT
TTATATTAATTTTATTGTTCAAACATGAGAGCTTAGTACGTGAAACATGAGAGCT
TAGTACATTAGCCATGAGAGCTTAGTACATTAGCCATGAGGGTTTAGTTCATTAA
ACATGAGAGCTTAGTACATTAAACATGAGAGCTTAGTACATACTATCAACAGGTT
GAACTGCTGATCTGTACAGTAGAATTGGTAAAGAGAGTTGTGTAAAATATTGAGT
TCGCACATCTTGTTGTCTGATTATTGATTTTTGGCGAAACCATTTGATCATATGAC
AAGATGTGTATCTACCTTAACTTAATGATTTTGATAAAAATCATTAGGTACCAAT
TACATTGCTTGCAATTAACCCTTTAACGGTTATAAGGATCTAGATGAGATAGAAA
GATTTGGTTTTCGGATTTGTGTTACATAAGATGCCTAAAATAAAAATTGAGATTC
AATTTTTTTTAAACTTTTTTTTAATTGGTGGTAAGAATATTCCCTCTACCTGTTTGA
GAGTAATGAAATTGTAGTATGATTTTTCAACAAACTAAAAAAACAACATAAATCT
CACATAATAACTTTATTTCAATCACACAATTGAATACCAATAGGTTGACAGTACT
TACCAGCCTGCAGGTGAAAGACCCCACCTGTAGGTTTGGCAAGTTAGCTTAA
GTAACGCCATTTTGCAAGGCATGGAAAATACATAACTGAGAATAGAGAAGTT
CAGATCAAGGTTAGGAACAGAGAGACAGCAGAATATGGGCCAAACAGGATA
TCTGTGGTAAGCAGTTCCTGCCCCGGCTCAGGGCCAAGAACAGATGGTCCC
CAGATGCGGTCCCGCCCTCAGCAGTTTCTAGCGAACCATCAGATGTTTCCAG
GGTGCCCCAAGGACCTGAAATGACCCTGTGCCTTATTTGAACTAACCAATCA
GTTTGCTTCTTGCTTCTGTTTGTGTGCTTCTGCTCCCTGAGCTCAATAAAAG
AGCCCACAACCCCTCACTTGGTGGGCCAGTCCTCTGATAGACTGTGTCCCCT
GGATACCCGTACGGTACCGCTAGCGCCACCATGGGACCCAGGCTCCTCTTCTGGG
CACTGCTTTGTCTCCTCGGAACAGGCCCAGTGGAGGCTGGAGTCACACAAAGTCCC
ACACACCTGATCAAAACGAGAGGACAGCAAGCGACTCTGAGATGCTCTCCTATCTC
TGGGCACACCAGTGTGTACTGGTACCAACAGGCCCTGGGTCTGGGCCTCCAGTTCC
TCCTTTGGTATGACGAGGGTGAAGAGAGAAACAGAGGAAACTTCCCTCCTAGATTT
TCAGGTCGCCAGTTCCCTAATTATAGCTCTGAGCTGAATGTGAACGCCTTGGAGCT
GGAGGACTCGGCCCTGTATCTCTGTGCTTCCTCACTTGGGCAATTGAACACAGAGG
CATTCTTTGGACAAGGCACCAGACTCACAGTTGTAGAAGATCTGAACAAGGTGTTC
CCTCCAGAGGTGGCCGTGTTCGAGCCTTCTAAGGCCGAGATCGCCCACACACAAAA
AGCCACCCTCGTGTGCCTGGCCACCGGCTTTTTCCCCGACCACGTGGAACTGTCTT
GGTGGGTCAACGGCAAAGAGGTGCACTCCGGCGTGTCAACGGATCCCCAGCCTCT
GAAAGAACAGCCTGCCCTGAACGACAGCCGGTACTGCCTGAGCTCCAGACTGAGA
GTGTCCGCCACCTTCTGGCAGAACCCCCGGAACCACTTCAGATGCCAGGTGCAGTT
TTACGGCCTGAGCGAGAACGACGAGTGGACCCAGGACAGAGCCAAGCCCGTGACA
CAAATCGTGTCTGCCGAAGCCTGGGGAAGAGCCGATTGCGGCATCACCAGCGCCT
CCTATCACCAGGGCGTGCTGAGCGCCACAATCCTGTACGAAATCCTGCTGGGCAAG
GCCACCCTGTACGCCGTGCTGGTGTCTGCTCTGGTGCTGATGGCCATGGTCAAGCG
GAAGGACTTTGGCAGCGGCAGAGCCAAAAGGTCCGGGAGCGGTGCGACAAACTT
TAGCCTGTTGAAACAAGCCGGCGACGTTGAAGAGAACCCCGGACCTATGGAAAA
AATGCTCGAGTGCGCCTTCATCGTGCTTTGGCTGCAGCTCGGATGGCTGAG
CGGAGAGGATCAAGTGACACAGTCTCCCGAGGCTCTGAGGCTGCAAGAGGG
CGAAAGCAGCTCCCTGAATTGCAGCTACACCGTGTCTGGCCTGAGGGGCCT
GTTTTGGTACAGACAAGACCCTGGCAAGGGACCCGAGTTCCTGTTCACACT
GTACTCTGCCGGCGAAGAAAAAGAGAAAGAGCGCCTGAAAGCAACCCTGAC
CAAGAAAGAGAGCTTCCTGCACATCACAGCCCCTAAGCCAGAGGACAGCGC
TACTTACCTGTGTGCCGTTTCATACGGCCAGAATTTCGTTTTTGGTCCCGGA
ACCAGATTGTCCGTGCTGCCCTACATCCAGAACCCCGACCCCGCCGTGTACC
AGCTGAGGGACTCCAAGTCCAGCGACAAGAGCGTGTGTCTGTTTACGGACT
TCGACAGCCAGACCAACGTGAGTCAAAGCAAGGACAGCGACGTCTACATAA
CGGATAAGACCGTGCTGGACATGCGGAGCATGGACTTCAAGAGCAACAGCG
CCGTGGCCTGGTCCAACAAGAGCGACTTCGCCTGCGCCAACGCCTTCAACA
ACAGCATCATCCCCGAGGACACCTTCTTCCCCAGCAGCGACGTGCCCTGCG
ACGTGAAACTGGTGGAGAAGTCCTTCGAGACAGACACCAATCTGAACTTTCA
GAACCTGCTGGTGATCGTGCTGCGGATTCTGCTGCTGAAAGTGGCCGGCTT
CAATCTGCTGATGACCCTGCGGCTGTGGAGCAGCAGGGCTAAGAGGTCCGGC
AGCGGAGCCACCAATTTTTCCCTGCTGAAACAGGCTGGTGACGTGGAAGAAAAC
CCTGGCCCCATGGCGCTGCCCGTCACCGCGCTGCTGCTGCCCCTGGCGCTGCTGTTA
CACGCCGCTCGGCCAGAGCTTCCCACCCAGGGCACATTCTCCAACGTGTCCACCAAT
GTGTCGGGAGGCGGCGGATCGTCCCAGTTCAGAGTGTCCCCTCTGGACCGCACCTGG
AACCTGGGCGAGACCGTGGAGCTGAAATGTCAGGTCCTGCTGAGCAACCCGACCTCC
GGGTGCAGTTGGCTGTTCCAGCCGCGTGGTGCTGCCGCAAGCCCTACGTTCCTGCTT
TACCTGAGCCAGAACAAGCCCAAGGCGGCCGAGGGCCTGGACACCCAGAGATTCTCC
GGCAAGCGCCTGGGGGACACATTCGTGCTTACTTTGAGCGATTTCCGCAGAGAGAAC
GAGGGCTACTATTTCTGTTCGGCGCTGAGCAATTCCATCATGTATTTCAGCCACTTTGT
GCCAGTGTTCCTGCCTGCCAAGCCTACCACAACACCAGCTCCCCGTCCCCCGACTCC
GGCGCCTACCATCGCGAGTCAACCGTTGAGCCTGAGGCCTGAGGCTTGTCGGCCCGC
TGCGGGGGGTGCCGTCCACACCAGGGGCCTCGACTTTGCGTGCGACATCTATATTTG
GGCGCCTCTGGCGGGTACCTGCGGGGTGCTGCTGCTGTCATTGGTGATTACCCTGTA
CTGCAATCACCGCAACCGCCGGCGGGTCTGTAAGTGCCCACGGCCTGTGGTCAAGTC
CGGTGACAAACCGTCGCTCTCGGCTCGCTACGTGCGCGCTAAGCGCAGCGGTTCCG
GGGCCACCAACTTTTCATTGCTGAAGCAGGCCGGTGATGTGGAGGAGAATCCAG
GGCCCATGCGCCCCAGGCTTTGGCTCCTTCTTGCTGCTCAGCTCACTGTCTTGCAT
GGCAACTCCGTTCTGCAGCAGACTCCCGCCTACATCAAGGTGCAGACGAACAAG
ATGGTGATGCTGTCATGCGAGGCCAAGATCTCTCTTTCAAATATGAGAATTTATT
GGCTACGACAGCGCCAGGCCCCCTCCAGCGACAGCCACCACGAGTTCCTGGCGC
TTTGGGATTCTGCTAAAGGCACCATCCATGGAGAGGAGGTGGAACAGGAGAAGA
TAGCTGTCTTCCGCGACGCATCCCGCTTCATCCTGAACCTGACCAGCGTGAAGCC
GGAGGACAGCGGCATCTACTTCTGTATGATCGTTGGCTCCCCCGAGCTGACCTTC
GGCAAAGGCACCCAGCTGTCCGTGGTGGACTTCCTGCCCACCACAGCCCAGCCA
ACCAAGAAATCCACCCTCAAGAAGCGCGTGTGCCGACTGCCCCGCCCTGAAACC
CAGAAGGGCCCTCTGTGCTCCCCCATCACCCTTGGACTGCTGGTGGCGGGAGTCC
TGGTGCTGCTCGTATCTCTGGGTGTCGCCATCCACCTGTGCTGCCGCCGCCGCCG
CGCCCGCCTGAGGTTTATGAAACAGTTTTACAAGTGATAAATCGATGGAAGGGT
GGCATCCCTGTGACCCCTCCCCAGTGCCTCTCCTGGCCCTGGAAGTTGCCACTCC
AGTGCCCACCAGCCTTGTCCTAATAAAATTAAGTTGCATCATTTTGTCTGACTAG
GTGTCCTTCTATAATATTATGGGGTGGAGGGGGGTGGTATGGAGCAAGGGGCAA
GTTGGGAAGACAACCTGTAGGGCCTGCGGGGTCTATTGGGAACCAAGCTGGAGT
GCAGTGGCACAATCTTGGCTCACTGCAATCTCCGCCTCCTGGGTTCAAGCGATTC
TCCTGCCTCAGCCTCCCGAGTTGTTGGGATTCCAGGCATGCATGACCAGGCTCAG
CTAATTTTTGTTTTTTTGGTAGAGACGGGGTTTCACCATATTGGCCAGGCTGGTCT
CCAACTCCTAATCTCAGGTGATCTACCCACCTTGGCCTCCCAAATTGCTGGGATT
ACAGGCGTGAACCACTGCTCCCTTCCCTGTCCTTCTGATTACTAGTGGCTCCGGTG
CCCGTCAGTGGGCAGAGCGCACATCGCCCACAGTCCCCGAGAAGTTGTGGGGAG
GGGTCGGCAATTGAACCGGTGCCTAGAGAAGGTGGCGCGGGGTAAACTGGGAAA
GTGATGTCGTGTACTGGCTCCGCCTTTTTCCCGAGGGTGGGGGAGAACCGTATAT
AAGTGCAGTAGTCGCCGTGAACGTTCTTTTTCGCAACGGGTTTGCCGCCAGAACA
CAGGTAAGTGCCGTGTGTGGTTCCCGCGGGCCTGGCCTCTTTACGGGTTATGGCC
CTTGCGTGCCTTGAATTACTTCCACCTGGCTGCAGTACGTGATTCTTGATCCCGAG
CTTCGGGTTGGAAGTGGGTGGGAGAGTTCGAGGCCTTGCGCTTAAGGAGCCCCTT
CGCCTCGTGCTTGAGTTGAGGCCTGGCCTGGGCGCTGGGGCCGCCGCGTGCGAAT
CTGGTGGCACCTTCGCGCCTGTCTCGCTGCTTTCGATAAGTCTCTAGCCATTTAAA
ATTTTTGATGACCTGCTGCGACGCTTTTTTTCTGGCAAGATAGTCTTGTAAATGCG
GGCCAAGATCTGCACACTGGTATTTCGGTTTTTGGGGCCGCGGGCGGCGACGGG
GCCCGTGCGTCCCAGCGCACATGTTCGGCGAGGCGGGGCCTGCGAGCGCGGCCA
CCGAGAATCGGACGGGGGTAGTCTCAAGCTGGCCGGCCTGCTCTGGTGCCTGGC
CTCGCGCCGCCGTGTATCGCCCCGCCCTGGGCGGCAAGGCTGGCCCGGTCGGCAC
CAGTTGCGTGAGCGGAAAGATGGCCGCTTCCCGGCCCTGCTGCAGGGAGCTCAA
AATGGAGGACGCGGCGCTCGGGAGAGCGGGCGGGTGAGTCACCCACACAAAGG
AAAAGGGCCTTTCCGTCCTCAGCCGTCGCTTCATGTGACTCCACGGAGTACCGGG
CGCCGTCCAGGCACCTCGATTAGTTCTCGAGCTTTTGGAGTACGTCGTCTTTAGGT
TGGGGGGAGGGGTTTTATGCGATGGAGTTTCCCCACACTGAGTGGGTGGAGACT
GAAGTTAGGCCAGCTTGGCACTTGATGTAATTCTCCTTGGAATTTGCCCTTTTTGA
GTTTGGATCTTGGTTCATTCTCAAGCCTCAGACAGTGGTTCAAAGTTTTTTTCTTC
CATTTCAGGTGTCGTGAACTAGTCCAGTGTGGTGGAATTCTGCAGATATCACGGC
TAGCGCCACCATGGGTCGGGGGCTGCTCAGGGGCCTGTGGCCGCTGCACATCGTC
CTGTGGACGCGTATCGCCAGCACGATCCCACCGCACGTTCAGAAGTCGGTGAAT
AACGACATGATAGTCACTGACAACAACGGTGCAGTCAAGTTTCCACAACTGTGT
AAATTTTGTGATGTGAGATTTTCCACCTGTGACAACCAGAAATCCTGCATGAGCA
ACTGCAGCATCACCTCCATCTGTGAGAAGCCACAGGAAGTCTGTGTGGCTGTATG
GAGAAAGAATGACGAGAACATAACACTAGAGACAGTTTGCCATGACCCCAAGCT
CCCCTACCATGACTTTATTCTGGAAGATGCTGCTTCTCCAAAGTGCATTATGAAG
GAGAAGAAAAAGCCTGGTGAGACTTTCTTCATGTGTTCCTGTAGCTCTGATGAGT
GCAATGACAACATCATCTTCTCAGAAGAATATAACACCAGCAATCCTGACTTGTT
GCTAGTCATATTTCAAGTGACAGGCATCAGCCTCCTGCCACCACTGGGAGTTGCC
ATATCTGTCATCATCATCTTCTACTGCTACCGCGTGAACCGGCAGCAGAAGGCTA
GTGGTTCAGGCGCAACGAATTTCTCTTTGCTGAAGCAGGCTGGGGATGTCGAAGA
AAATCCGGGTCCAATGGTGGGCTCGCTCAACTGCATCGTAGCAGTCTCCCAGAAT
ATGGGCATCGGGAAGAACGGTGATTTCCCGTGGCCCCCACTTCGCAACGAGAGC
CGTTATTTCCAAAGAATGACTACAACCTCCTCCGTGGAGGGTAAGCAGAACCTGG
TCATCATGGGGAAGAAGACCTGGTTCTCTATCCCTGAAAAAAACCGCCCCCTGAA
GGGCCGCATCAACCTGGTGCTGAGCAGGGAACTCAAGGAGCCTCCTCAGGGCGC
GCATTTTCTGAGCCGCTCATTGGATGACGCTCTCAAACTGACCGAACAGCCGGAG
CTAGCCAACAAGGTGGACATGGTGTGGATCGTCGGAGGCTCCTCCGTGTACAAG
GAGGCCATGAATCACCCCGGCCACTTGAAGCTGTTCGTCACCCGGATCATGCAGG
ACTTCGAGTCGGACACGTTCTTTCCAGAGATTGACCTGGAGAAGTACAAGCTGCT
GCCCGAGTACCCGGGAGTTCTTAGTGATGTGCAGGAGGAGAAAGGCATCAAGTA
CAAATTTGAGGTGTACGAGAAGAACGACTAACGGTCCGTCCTGACCAATGCTGG
AGTTCTTCGCCCACCCCAACTTGTTTATTGCAGCTTATAATGGTTACAAATAAAG
CAATAGCATCACAAATTTCACAAATAAAGCATTTTTTTCACTGCATTCTAGTTGTG
GTTTGTCCAAACTCATCAATGTATCTTATCATGTCTGTATACAGGTTACCTCAGTC
TCCTAGGTACGTCTTATATCTATGAAAAAACATTCAAAAGCACAACATCTAGAAG
AACTTACCTTTTTTCACCACTCTATTGCAAAGATATGTACCGATTTCTCTCGAAGT
ACAAAAAACCGCTAGTTTTCAAATTCACCTCAAGACTTTGAAAAAAAATTGAATC
TGTCAATGTCAAATAAAATCAGAAACAAATGTCATAATGTTACGTTAATGTTGTC
AGGTCGAAAAATAAAATTGCAAATAGAAATTTTGTTCCTTTTTTATTGGTTTTTAT
TGGTGGGAAAAATATTCCCTCTAACTGCAAAAGGGTTAATTATGTTAGAGGTAGA
GTCGACAAGCTT (SEQ ID NO: 20)
Representative Vector (the TCR-encoding protein of which can be interchanged with
any TCR sequence of interest): pNVVD166_TSC-204-A02_TCR-1479_MSCV-TCR-
1479-CD8-EF1a-DHFR
GCTAGCTGGCTTGTTGTCCACAACCATTAAACCTTAAAAGCTTTAAAAGCCTTAT
ATATTCTTTTTTTTCTTATAAAACTTAAAACCTTAGAGGCTATTTAAGTTGCTGAT
TTATATTAATTTTATTGTTCAAACATGAGAGCTTAGTACGTGAAACATGAGAGCT
TAGTACATTAGCCATGAGAGCTTAGTACATTAGCCATGAGGGTTTAGTTCATTAA
ACATGAGAGCTTAGTACATTAAACATGAGAGCTTAGTACATACTATCAACAGGTT
GAACTGCTGATCTGTACAGTAGAATTGGTAAAGAGAGTTGTGTAAAATATTGAGT
TCGCACATCTTGTTGTCTGATTATTGATTTTTGGCGAAACCATTTGATCATATGAC
AAGATGTGTATCTACCTTAACTTAATGATTTTGATAAAAATCATTAGGTACCAAT
TACATTGCTTGCAATTAACCCTTTAACGGTTATAAGGATCTAGATGAGATAGAAA
GATTTGGTTTTCGGATTTGTGTTACATAAGATGCCTAAAATAAAAATTGAGATTC
AATTTTTTTTAAACTTTTTTTTAATTGGTGGTAAGAATATTCCCTCTACCTGTTTGA
GAGTAATGAAATTGTAGTATGATTTTTCAACAAACTAAAAAAACAACATAAATCT
CACATAATAACTTTATTTCAATCACACAATTGAATACCAATAGGTTGACAGTACT
TACCAGCCTGCAGGTGAAAGACCCCACCTGTAGGTTTGGCAAGTTAGCTTAA
GTAACGCCATTTTGCAAGGCATGGAAAATACATAACTGAGAATAGAGAAGTT
CAGATCAAGGTTAGGAACAGAGAGACAGCAGAATATGGGCCAAACAGGATA
TCTGTGGTAAGCAGTTCCTGCCCCGGCTCAGGGCCAAGAACAGATGGTCCC
CAGATGCGGTCCCGCCCTCAGCAGTTTCTAGCGAACCATCAGATGTTTCCAG
GGTGCCCCAAGGACCTGAAATGACCCTGTGCCTTATTTGAACTAACCAATCA
GTTTGCTTCTTGCTTCTGTTTGTGTGCTTCTGCTCCCTGAGCTCAATAAAAG
AGCCCACAACCCCTCACTTGGTGGGCCAGTCCTCTGATAGACTGTGTCCCCT
GGATACCCGTACGGTACCGCTAGCGCCACCATGGGACCCAGGCTCCTCTTCTGGG
CACTGCTTTGTCTCCTCGGAACAGGCCCAGTGGAGGCTGGAGTCACACAAAGTCCC
ACACACCTGATCAAAACGAGAGGACAGCAAGCGACTCTGAGATGCTCTCCTATCTC
TGGGCACACCAGTGTGTACTGGTACCAACAGGCCCTGGGTCTGGGCCTCCAGTTCC
TCCTTTGGTATGACGAGGGTGAAGAGAGAAACAGAGGAAACTTCCCTCCTAGATTT
TCAGGTCGCCAGTTCCCTAATTATAGCTCTGAGCTGAATGTGAACGCCTTGGAGCT
GGAGGACTCGGCCCTGTATCTCTGTGCTTCCTCACTTGGGCAATTGAACACAGAGG
CATTCTTTGGACAAGGCACCAGACTCACAGTTGTAGAAGATCTGAACAAGGTGTTC
CCTCCAGAGGTGGCCGTGTTCGAGCCTTCTAAGGCCGAGATCGCCCACACACAAAA
AGCCACCCTCGTGTGCCTGGCCACCGGCTTTTTCCCCGACCACGTGGAACTGTCTT
GGTGGGTCAACGGCAAAGAGGTGCACTCCGGCGTGTCAACGGATCCCCAGCCTCT
GAAAGAACAGCCTGCCCTGAACGACAGCCGGTACTGCCTGAGCTCCAGACTGAGA
GTGTCCGCCACCTTCTGGCAGAACCCCCGGAACCACTTCAGATGCCAGGTGCAGTT
TTACGGCCTGAGCGAGAACGACGAGTGGACCCAGGACAGAGCCAAGCCCGTGACA
CAAATCGTGTCTGCCGAAGCCTGGGGAAGAGCCGATTGCGGCATCACCAGCGCCT
CCTATCACCAGGGCGTGCTGAGCGCCACAATCCTGTACGAAATCCTGCTGGGCAAG
GCCACCCTGTACGCCGTGCTGGTGTCTGCTCTGGTGCTGATGGCCATGGTCAAGCG
GAAGGACTTTGGCAGCGGCAGAGCCAAAAGGTCCGGGAGCGGTGCGACAAACTT
TAGCCTGTTGAAACAAGCCGGCGACGTTGAAGAGAACCCCGGACCTATGGAAAA
AATGCTCGAGTGCGCCTTCATCGTGCTTTGGCTGCAGCTCGGATGGCTGAG
CGGAGAGGATCAAGTGACACAGTCTCCCGAGGCTCTGAGGCTGCAAGAGGG
CGAAAGCAGCTCCCTGAATTGCAGCTACACCGTGTCTGGCCTGAGGGGCCT
GTTTTGGTACAGACAAGACCCTGGCAAGGGACCCGAGTTCCTGTTCACACT
GTACTCTGCCGGCGAAGAAAAAGAGAAAGAGCGCCTGAAAGCAACCCTGAC
CAAGAAAGAGAGCTTCCTGCACATCACAGCCCCTAAGCCAGAGGACAGCGC
TACTTACCTGTGTGCCGTTTCATACGGCCAGAATTTCGTTTTTGGTCCCGGA
ACCAGATTGTCCGTGCTGCCCTACATCCAGAACCCCGACCCCGCCGTGTACC
AGCTGAGGGACTCCAAGTCCAGCGACAAGAGCGTGTGTCTGTTTACGGACT
TCGACAGCCAGACCAACGTGAGTCAAAGCAAGGACAGCGACGTCTACATAA
CGGATAAGACCGTGCTGGACATGCGGAGCATGGACTTCAAGAGCAACAGCG
CCGTGGCCTGGTCCAACAAGAGCGACTTCGCCTGCGCCAACGCCTTCAACA
ACAGCATCATCCCCGAGGACACCTTCTTCCCCAGCAGCGACGTGCCCTGCG
ACGTGAAACTGGTGGAGAAGTCCTTCGAGACAGACACCAATCTGAACTTTCA
GAACCTGCTGGTGATCGTGCTGCGGATTCTGCTGCTGAAAGTGGCCGGCTT
CAATCTGCTGATGACCCTGCGGCTGTGGAGCAGCAGGGCTAAGAGGTCCGGC
AGCGGAGCCACCAATTTTTCCCTGCTGAAACAGGCTGGTGACGTGGAAGAAAAC
CCTGGCCCCATGGCGCTGCCCGTCACCGCGCTGCTGCTGCCCCTGGCGCTGCTGTTA
CACGCCGCTCGGCCAGAGCTTCCCACCCAGGGCACATTCTCCAACGTGTCCACCAAT
GTGTCGGGAGGCGGCGGATCGTCCCAGTTCAGAGTGTCCCCTCTGGACCGCACCTGG
AACCTGGGCGAGACCGTGGAGCTGAAATGTCAGGTCCTGCTGAGCAACCCGACCTCC
GGGTGCAGTTGGCTGTTCCAGCCGCGTGGTGCTGCCGCAAGCCCTACGTTCCTGCTT
TACCTGAGCCAGAACAAGCCCAAGGCGGCCGAGGGCCTGGACACCCAGAGATTCTCC
GGCAAGCGCCTGGGGGACACATTCGTGCTTACTTTGAGCGATTTCCGCAGAGAGAAC
GAGGGCTACTATTTCTGTTCGGCGCTGAGCAATTCCATCATGTATTTCAGCCACTTTGT
GCCAGTGTTCCTGCCTGCCAAGCCTACCACAACACCAGCTCCCCGTCCCCCGACTCC
GGCGCCTACCATCGCGAGTCAACCGTTGAGCCTGAGGCCTGAGGCTTGTCGGCCCGC
TGCGGGGGGTGCCGTCCACACCAGGGGCCTCGACTTTGCGTGCGACATCTATATTTG
GGCGCCTCTGGCGGGTACCTGCGGGGTGCTGCTGCTGTCATTGGTGATTACCCTGTA
CTGCAATCACCGCAACCGCCGGCGGGTCTGTAAGTGCCCACGGCCTGTGGTCAAGTC
CGGTGACAAACCGTCGCTCTCGGCTCGCTACGTGCGCGCTAAGCGCAGCGGTTCCG
GGGCCACCAACTTTTCATTGCTGAAGCAGGCCGGTGATGTGGAGGAGAATCCAG
GGCCCATGCGCCCCAGGCTTTGGCTCCTTCTTGCTGCTCAGCTCACTGTCTTGCAT
GGCAACTCCGTTCTGCAGCAGACTCCCGCCTACATCAAGGTGCAGACGAACAAG
ATGGTGATGCTGTCATGCGAGGCCAAGATCTCTCTTTCAAATATGAGAATTTATT
GGCTACGACAGCGCCAGGCCCCCTCCAGCGACAGCCACCACGAGTTCCTGGCGC
TTTGGGATTCTGCTAAAGGCACCATCCATGGAGAGGAGGTGGAACAGGAGAAGA
TAGCTGTCTTCCGCGACGCATCCCGCTTCATCCTGAACCTGACCAGCGTGAAGCC
GGAGGACAGCGGCATCTACTTCTGTATGATCGTTGGCTCCCCCGAGCTGACCTTC
GGCAAAGGCACCCAGCTGTCCGTGGTGGACTTCCTGCCCACCACAGCCCAGCCA
ACCAAGAAATCCACCCTCAAGAAGCGCGTGTGCCGACTGCCCCGCCCTGAAACC
CAGAAGGGCCCTCTGTGCTCCCCCATCACCCTTGGACTGCTGGTGGGGGAGTCC
TGGTGCTGCTCGTATCTCTGGGTGTCGCCATCCACCTGTGCTGCCGCCGCCGCCG
CGCCCGCCTGAGGTTTATGAAACAGTTTTACAAGTGATAAATCGATGGAAGGGT
GGCATCCCTGTGACCCCTCCCCAGTGCCTCTCCTGGCCCTGGAAGTTGCCACTCC
AGTGCCCACCAGCCTTGTCCTAATAAAATTAAGTTGCATCATTTTGTCTGACTAG
GTGTCCTTCTATAATATTATGGGGTGGAGGGGGGTGGTATGGAGCAAGGGGCAA
GTTGGGAAGACAACCTGTAGGGCCTGCGGGGTCTATTGGGAACCAAGCTGGAGT
GCAGTGGCACAATCTTGGCTCACTGCAATCTCCGCCTCCTGGGTTCAAGCGATTC
TCCTGCCTCAGCCTCCCGAGTTGTTGGGATTCCAGGCATGCATGACCAGGCTCAG
CTAATTTTTGTTTTTTTGGTAGAGACGGGGTTTCACCATATTGGCCAGGCTGGTCT
CCAACTCCTAATCTCAGGTGATCTACCCACCTTGGCCTCCCAAATTGCTGGGATT
ACAGGCGTGAACCACTGCTCCCTTCCCTGTCCTTCTGATTACTAGTGGCTCCGGTG
CCCGTCAGTGGGCAGAGCGCACATCGCCCACAGTCCCCGAGAAGTTGTGGGGAG
GGGTCGGCAATTGAACCGGTGCCTAGAGAAGGTGGCGCGGGGTAAACTGGGAAA
GTGATGTCGTGTACTGGCTCCGCCTTTTTCCCGAGGGTGGGGGAGAACCGTATAT
AAGTGCAGTAGTCGCCGTGAACGTTCTTTTTCGCAACGGGTTTGCCGCCAGAACA
CAGGTAAGTGCCGTGTGTGGTTCCCGCGGGCCTGGCCTCTTTACGGGTTATGGCC
CTTGCGTGCCTTGAATTACTTCCACCTGGCTGCAGTACGTGATTCTTGATCCCGAG
CTTCGGGTTGGAAGTGGGTGGGAGAGTTCGAGGCCTTGCGCTTAAGGAGCCCCTT
CGCCTCGTGCTTGAGTTGAGGCCTGGCCTGGGCGCTGGGGCCGCCGCGTGCGAAT
CTGGTGGCACCTTCGCGCCTGTCTCGCTGCTTTCGATAAGTCTCTAGCCATTTAAA
ATTTTTGATGACCTGCTGCGACGCTTTTTTTCTGGCAAGATAGTCTTGTAAATGCG
GGCCAAGATCTGCACACTGGTATTTCGGTTTTTGGGGCCGCGGGCGGCGACGGG
GCCCGTGCGTCCCAGCGCACATGTTCGGCGAGGCGGGGCCTGCGAGCGCGGCCA
CCGAGAATCGGACGGGGGTAGTCTCAAGCTGGCCGGCCTGCTCTGGTGCCTGGC
CTCGCGCCGCCGTGTATCGCCCCGCCCTGGGCGGCAAGGCTGGCCCGGTCGGCAC
CAGTTGCGTGAGCGGAAAGATGGCCGCTTCCCGGCCCTGCTGCAGGGAGCTCAA
AATGGAGGACGCGGCGCTCGGGAGAGCGGGCGGGTGAGTCACCCACACAAAGG
AAAAGGGCCTTTCCGTCCTCAGCCGTCGCTTCATGTGACTCCACGGAGTACCGGG
CGCCGTCCAGGCACCTCGATTAGTTCTCGAGCTTTTGGAGTACGTCGTCTTTAGGT
TGGGGGGAGGGGTTTTATGCGATGGAGTTTCCCCACACTGAGTGGGTGGAGACT
GAAGTTAGGCCAGCTTGGCACTTGATGTAATTCTCCTTGGAATTTGCCCTTTTTGA
GTTTGGATCTTGGTTCATTCTCAAGCCTCAGACAGTGGTTCAAAGTTTTTTTCTTC
CATTTCAGGTGTCGTGAGCCACCATGGTGGGCTCGCTCAACTGCATCGTAGCAGT
CTCCCAGAATATGGGCATCGGGAAGAACGGTGATTTCCCGTGGCCCCCACTTCGC
AACGAGAGCCGTTATTTCCAAAGAATGACTACAACCTCCTCCGTGGAGGGTAAG
CAGAACCTGGTCATCATGGGGAAGAAGACCTGGTTCTCTATCCCTGAAAAAAAC
CGCCCCCTGAAGGGCCGCATCAACCTGGTGCTGAGCAGGGAACTCAAGGAGCCT
CCTCAGGGCGCGCATTTTCTGAGCCGCTCATTGGATGACGCTCTCAAACTGACCG
AACAGCCGGAGCTAGCCAACAAGGTGGACATGGTGTGGATCGTCGGAGGCTCCT
CCGTGTACAAGGAGGCCATGAATCACCCCGGCCACTTGAAGCTGTTCGTCACCCG
GATCATGCAGGACTTCGAGTCGGACACGTTCTTTCCAGAGATTGACCTGGAGAAG
TACAAGCTGCTGCCCGAGTACCCGGGAGTTCTTAGTGATGTGCAGGAGGAGAAA
GGCATCAAGTACAAATTTGAGGTGTACGAGAAGAACGACTAACGGTCCGTCCTG
ACCAATGCTGGAGTTCTTCGCCCACCCCAACTTGTTTATTGCAGCTTATAATGGTT
ACAAATAAAGCAATAGCATCACAAATTTCACAAATAAAGCATTTTTTTCACTGCA
TTCTAGTTGTGGTTTGTCCAAACTCATCAATGTATCTTATCATGTCTGTATACAGG
TTACCTCAGTCTCCTAGGTACGTCTTATATCTATGAAAAAACATTCAAAAGCACA
ACATCTAGAAGAACTTACCTTTTTTCACCACTCTATTGCAAAGATATGTACCGATT
TCTCTCGAAGTACAAAAAACCGCTAGTTTTCAAATTCACCTCAAGACTTTGAAAA
AAAATTGAATCTGTCAATGTCAAATAAAATCAGAAACAAATGTCATAATGTTAC
GTTAATGTTGTCAGGTCGAAAAATAAAATTGCAAATAGAAATTTTGTTCCTTTTTT
ATTGGTTTTTATTGGTGGGAAAAATATTCCCTCTAACTGCAAAAGGGTTAATTAT
GTTAGAGGTAGAGTCGAC (SEQ ID NO: 21)
*For certain depicted vectors, MSCV promoter is in bold. Beta chain is annotated using bold and italic text. Alpha chain is annotated using bold and underlined text. CD34-enrichment tag (Q tag) is annotated using italic and underlined text. CD8-alpha is in italic. CD8-beta is underlined.

TABLE 4
T-Knife-based T1367 TCR MGTM codon optimized sequence
Alpha chain:
TRAV5/TRAJ41/MGTM modified TRAC
Alpha chain DNA sequence
ATGAAGACCTTCGCCGGCTTCAGCTTCCTGTTCCTGTGGCTGCAGCTGGACTGCA
TGAGCAGGGGCGAGGACGTGGAACAGAGCCTGTTTCTGAGCGTGCGCGAGGGCG
ACAGCAGCGTGATCAATTGCACCTACACCGACAGCTCCAGCACCTACCTGTACT
GGTACAAGCAGGAACCTGGCGCCGGACTGCAGCTGCTGACCTACATCTTCAGCA
ACATGGACATGAAGCAGGACCAGAGACTGACCGTGCTGCTGAACAAGAAGGAC
AAGCACCTGAGCCTGCGGATCGCCGATACCCAGACAGGCGACAGCGCCATCTAC
TTTTGCGCCGAGAGCATCGGCAGCAACAGCGGCTACGCCCTGAACTTCGGCA
AGGGCACAAGCCTGCTCGTGACCCCTCacatccagaaccccgaccccgccgtgtaccagctgagggactc
caagtccagcgacaagagcgtgtgtctgtttacggacttcgacagccagaccaacgtgagtcaaagcaaggacagcgacgtctacat
aacggataagaccgtgctggacatgcggagcatggacttcaagagcaacagcgccgtggcctggtccaacaagagcgacttcgcct
gcgccaacgccttcaacaacagcatcatccccgaggacaccttcttccccagcagcgacgtgccctgcgacgtgaaactggtggag
aagtccttcgagacagacaccaatctgaactttcagaacctgctggtgatcgtgctgcggattctgctgctgaaagtggccggcttcaat
ctgctgatgaccctgcggctgtggagc (SEQ ID NO: 22)
Alpha chain protein sequence
MKTFAGFSFLFLWLQLDCMSRGEDVEQSLFLSVREGDSSVINCTYTDSSSTYLYWYK
QEPGAGLQLLTYIFSNMDMKQDQRLTVLLNKKDKHLSLRIADTQTGDSAIYFCAESI
GSNSGYALNFGKGTSLLVTPHiqnpdpavyqlrdskssdksvclftdfdsqtnvsqskdsdvyitdktvldmrs
mdfksnsavawsnksdfacanafnnsiipedtffpssdvpcdvklveksfetdtnlnfqnllvivlrilllkvagfnllmtlrlws
(SEQ ID NO: 23)
Beta chain:
TRBV28/TRBJ2-7/MGTM modified TRBC
Beta chain DNA sequence
ATGGGAATCAGACTGCTGTGCAGAGTGGCCTTCTGCTTCCTGGCCGTGGGCCTGG
TGGACGTGAAAGTGACCCAGAGCAGCAGATACCTCGTGAAGCGGACCGGCGAGA
AGGTGTTCCTGGAATGCGTGCAGGACATGGACCACGAGAATATGTTCTGGTACA
GACAGGACCCCGGCCTGGGCCTGCGGCTGATCTACTTCAGCTACGACGTGAAGA
TGAAGGAAAAGGGCGACATCCCCGAGGGCTACAGCGTGTCCAGAGAGAAGAAA
GAGCGGTTCAGCCTGATCCTGGAAAGCGCCAGCACCAACCAGACCAGCATGTAC
CTGTGCGCCAGCAGAGGCCTGGCCGGCTACGAGCAGTATTTTGGCCCTGGCA
CCCGGCTGACCGTGACCGaagatctgaacaaggtgttccctccagaggtggccgtgttcgagccttctaaggccgag
atcgcccacacacaaaaagccaccctcgtgtgcctggccaccggctttttccccgaccacgtggaactgtcttggtgggtcaacggca
aagaggtgcactccggcgtgtcaacggatccccagcctctgaaagaacagcctgccctgaacgacagccggtactgcctgagctcc
agactgagagtgtccgccaccttctggcagaacccccggaaccacttcagatgccaggtgcagttttacggcctgagcgagaacgac
gagtggacccaggacagagccaagcccgtgacacaaatcgtgtctgccgaagcctggggaagagccgattgcggcatcaccagc
gcctcctatcaccagggcgtgctgagcgccacaatcctgtacgaaatcctgctgggcaaggccaccctgtacgccgtgctggtgtctg
ctctggtgctgatggccatggtcaagcggaaggactttggcagcggcagagccaaaaggtccgggagcggt (SEQ ID NO:
24)
Beta chain protein sequence
MGIRLLCRVAFCFLAVGLVDVKVTQSSRYLVKRTGEKVFLECVQDMDHENMFWYR
QDPGLGLRLIYFSYDVKMKEKGDIPEGYSVSREKKERFSLILESASTNQTSMYLCASR
GLAGYEQYFGPGTRLTVTEdlnkvfppevavfepskaeiahtqkatlvclatgffpdhvelswwvngkevhsgv
stdpqplkeqpalndsryclssrlrvsatfwqnprnhfrcqvqfyglsendewtqdrakpvtqivsaeawgradcgitsasyhqgv
lsatilyeillgkatlyavlvsalvlmamvkrkdfgsgrakrsgsg (SEQ ID NO: 25)
Complete Beta and Alpha ORF DNA Sequence (The underlined italic region in the “Furin-
P2A” site encodes a sequence allowing for expression of two polypeptide chains in a single
cassette)
ATGGGAATCAGACTGCTGTGCAGAGTGGCCTTCTGCTTCCTGGCCGTGGGCCTGG
TGGACGTGAAAGTGACCCAGAGCAGCAGATACCTCGTGAAGCGGACCGGCGAGA
AGGTGTTCCTGGAATGCGTGCAGGACATGGACCACGAGAATATGTTCTGGTACA
GACAGGACCCCGGCCTGGGCCTGCGGCTGATCTACTTCAGCTACGACGTGAAGA
TGAAGGAAAAGGGCGACATCCCCGAGGGCTACAGCGTGTCCAGAGAGAAGAAA
GAGCGGTTCAGCCTGATCCTGGAAAGCGCCAGCACCAACCAGACCAGCATGTAC
CTGTGCGCCAGCAGAGGCCTGGCCGGCTACGAGCAGTATTTTGGCCCTGGCA
CCCGGCTGACCGTGACCGaagatctgaacaaggtgttccctccagaggtggccgtgttcgagccttctaaggccgag
atcgcccacacacaaaaagccaccctcgtgtgcctggccaccggctttttccccgaccacgtggaactgtcttggtgggtcaacggca
aagaggtgcactccggcgtgtcaacggatccccagcctctgaaagaacagcctgccctgaacgacagccggtactgcctgagctcc
agactgagagtgtccgccaccttctggcagaacccccggaaccacttcagatgccaggtgcagttttacggcctgagcgagaacgac
gagtggacccaggacagagccaagcccgtgacacaaatcgtgtctgccgaagcctggggaagagccgattgcggcatcaccagc
gcctcctatcaccagggcgtgctgagcgccacaatcctgtacgaaatcctgctgggcaaggccaccctgtacgccgtgctggtgtctg
ctctggtgctgatggccatggtcaagcggaaggactttggcagcggcagagccaaaaggtccgggagcggtGCGACAAAC
TTTAGCCTGTTGAAACAAGCCGGCGACGTTGAAGAGAACCCCGGACCTATGAAGACC
TTCGCCGGCTTCAGCTTCCTGTTCCTGTGGCTGCAGCTGGACTGCATGAGCAGGG
GCGAGGACGTGGAACAGAGCCTGTTTCTGAGCGTGCGCGAGGGCGACAGCAGCG
TGATCAATTGCACCTACACCGACAGCTCCAGCACCTACCTGTACTGGTACAAGC
AGGAACCTGGCGCCGGACTGCAGCTGCTGACCTACATCTTCAGCAACATGGAC
ATGAAGCAGGACCAGAGACTGACCGTGCTGCTGAACAAGAAGGACAAGCACCT
GAGCCTGCGGATCGCCGATACCCAGACAGGCGACAGCGCCATCTACTTTTGCGC
CGAGAGCATCGGCAGCAACAGCGGCTACGCCCTGAACTTCGGCAAGGGCAC
AAGCCTGCTCGTGACCCCTCacatccagaaccccgaccccgccgtgtaccagctgagggactccaagtccagcg
acaagagcgtgtgtctgtttacggacttcgacagccagaccaacgtgagtcaaagcaaggacagcgacgtctacataacggataaga
ccgtgctggacatgcggagcatggacttcaagagcaacagcgccgtggcctggtccaacaagagcgacttcgcctgcgccaacgc
cttcaacaacagcatcatccccgaggacaccttcttccccagcagcgacgtgccctgcgacgtgaaactggtggagaagtccttcgag
acagacaccaatctgaactttcagaacctgctggtgatcgtgctgcggattctgctgctgaaagtggccggcttcaatctgctgatgacc
ctgcggctgtggagc (SEQ ID NO: 26)
Complete Beta and Alpha ORF Protein Sequence (The underlined italic region in the “Furin-
P2A” site allows expression of two polypeptide chains in a single cassette)
MGIRLLCRVAFCFLAVGLVDVKVTQSSRYLVKRTGEKVFLECVQDMDHENMFWYR
QDPGLGLRLIYFSYDVKMKEKGDIPEGYSVSREKKERFSLILESASTNQTSMYLCASR
GLAGYEQYFGPGTRLTVTEdlnkvfppevavfepskaeiahtqkatlvclatgffpdhvelswwvngkevhsgv
stdpqplkeqpalndsryclssrlrvsatfwqnprnhfrcqvqfyglsendewtqdrakpvtqivsaeawgradcgitsasyhqgv
lsatilyeillgkatlyavlvsalvlmamvkrkdfgsgrakrsgsgATNFSLLKQAGDVEENPGPMKTFAGFSF
LFLWLQLDCMSRGEDVEQSLFLSVREGDSSVINCTYTDSSSTYLYWYKQEPGAGLQL
LTYIFSNMDMKQDQRLTVLLNKKDKHLSLRIADTQTGDSAIYFCAESIGSNSGYAL
NFGKGTSLLVTPHiqnpdpavyqlrdskssdksvclftdfdsqtnvsqskdsdvyitdktvldmrsmdfksnsavaws
nksdfacanafnnsiipedtffpssdvpcdvklveksfetdtnlnfqnllvivlrilllkvagfnllmtlrlws (SEQ ID NO:
27)
Immatics-based R37P1C9 TCR MGTM codon optimized sequence
Alpha chain:
TRAV26-2/TRAJ21/MGTM modified TRAC
Alpha chain DNA sequence
ATGAAGCTGGTGACCAGCATCACCGTGCTGCTGAGCCTGGGCATCATGGGCGAC
GCCAAGACCACCCAGCCCAACAGCATGGAGAGCAACGAGGAGGAGCCCGTGCA
CCTGCCCTGCAACCACAGCACCATCAGCGGCACCGACTACATCCACTGGTACA
GGCAGCTGCCCAGCCAGGGCCCCGAGTACGTGATCCACGGCCTGACCAGCAAC
GTGAACAACAGGATGGCCAGCCTGGCCATCGCCGAGGACAGGAAGAGCAGCAC
CCTGATCCTGCACAGGGCCACCCTGAGGGACGCCGCCGTGTACTACTGCATCCT
GTTCAACTTCAACAAGTTCTACTTCGGCAGCGGCACCAAGCTGAACGTGAAGC
CCAacatccagaaccccgaccccgccgtgtaccagctgagggactccaagtccagcgacaagagcgtgtgtctgtttacggacttc
gacagccagaccaacgtgagtcaaagcaaggacagcgacgtctacataacggataagaccgtgctggacatgcggagcatggactt
caagagcaacagcgccgtggcctggtccaacaagagcgacttcgcctgcgccaacgccttcaacaacagcatcatccccgaggac
accttcttccccagcagcgacgtgccctgcgacgtgaaactggtggagaagtccttcgagacagacaccaatctgaactttcagaacc
tgctggtgatcgtgctgcggattctgctgctgaaagtggccggcttcaatctgctgatgaccctgcggctgtggagc (SEQ ID
NO: 28)
Alpha chain protein sequence
MKLVTSITVLLSLGIMGDAKTTQPNSMESNEEEPVHLPCNHSTISGTDYIHWYRQLPS
QGPEYVIHGLTSNVNNRMASLAIAEDRKSSTLILHRATLRDAAVYYCILFNFNKFYF
GSGTKLNVKPNiqnpdpavyqlrdskssdksvclftdfdsqtnvsqskdsdvyitdktvldmrsmdfksnsavawsnk
sdfacanafnnsiipedtffpssdvpcdvklveksfetdtnlnfqnllvivlrilllkvagfnllmtlrlws (SEQ ID NO: 29)
Beta chain:
TRBV15/TRBJ1-4/MGTM modified TRBC
Beta chain DNA sequence
ATGGGCCCCGGCCTGCTGCACTGGATGGCCCTGTGCCTGCTGGGCACCGGCCACG
GCGACGCCATGGTGATCCAGAACCCCAGGTACCAGGTGACCCAGTTCGGCAAGC
CCGTGACCCTGAGCTGCAGCCAGACCCTGAACCACAACGTGATGTACTGGTACC
AGCAGAAGAGCAGCCAGGCCCCCAAGCTGCTGTTCCACTACTACGACAAGGAC
TTCAACAACGAGGCCGACACCCCCGACAACTTCCAGAGCAGGAGGCCCAACACC
AGCTTCTGCTTCCTGGACATCAGGAGCCCCGGCCTGGGCGACGCCGCCATGTACC
TGTGCGCCACCAGCAGCGGCGAGACCAACGAGAAGCTGTTCTTCGGCAGCG
GCACCCAGCTGAGCGTGCTGGaagatctgaacaaggtgttccctccagaggtggccgtgttcgagccttctaaggc
cgagatcgcccacacacaaaaagccaccctcgtgtgcctggccaccggctttttccccgaccacgtggaactgtcttggtgggtcaac
ggcaaagaggtgcactccggcgtgtcaacggatccccagcctctgaaagaacagcctgccctgaacgacagccggtactgcctgag
ctccagactgagagtgtccgccaccttctggcagaacccccggaaccacttcagatgccaggtgcagttttacggcctgagcgagaa
cgacgagtggacccaggacagagccaagcccgtgacacaaatcgtgtctgccgaagcctggggaagagccgattgcggcatcacc
agcgcctcctatcaccagggcgtgctgagcgccacaatcctgtacgaaatcctgctgggcaaggccaccctgtacgccgtgctggtg
tctgctctggtgctgatggccatggtcaagcggaaggactttggcagcggcagagccaaaaggtccgggagcggt (SEQ ID
NO: 30)
Beta chain protein sequence
MGPGLLHWMALCLLGTGHGDAMVIQNPRYQVTQFGKPVTLSCSQTLNHNVMYWY
QQKSSQAPKLLFHYYDKDFNNEADTPDNFQSRRPNTSFCFLDIRSPGLGDAAMYLCA
TSSGETNEKLFFGSGTQLSVLEdlnkvfppevavfepskaeiahtqkatlvclatgffpdhvelswwvngkevh
sgvstdpqplkeqpalndsryclssrlrvsatfwqnprnhfrcqvqfyglsendewtqdrakpvtqivsaeawgradcgitsasyh
qgvlsatilyeillgkatlyavlvsalvlmamvkrkdfgsgrakrsgsg (SEQ ID NO: 31)
Complete Beta and Alpha ORF DNA Sequence (The underlined italic region in the “Furin-
P2A” site encodes a sequence allowing for expression of two polypeptide chains in a single
cassette)
ATGGGCCCCGGCCTGCTGCACTGGATGGCCCTGTGCCTGCTGGGCACCGGCCACG
GCGACGCCATGGTGATCCAGAACCCCAGGTACCAGGTGACCCAGTTCGGCAAGC
CCGTGACCCTGAGCTGCAGCCAGACCCTGAACCACAACGTGATGTACTGGTACC
AGCAGAAGAGCAGCCAGGCCCCCAAGCTGCTGTTCCACTACTACGACAAGGAC
TTCAACAACGAGGCCGACACCCCCGACAACTTCCAGAGCAGGAGGCCCAACACC
AGCTTCTGCTTCCTGGACATCAGGAGCCCCGGCCTGGGCGACGCCGCCATGTACC
TGTGCGCCACCAGCAGCGGCGAGACCAACGAGAAGCTGTTCTTCGGCAGCG
GCACCCAGCTGAGCGTGCTGGaagatctgaacaaggtgttccctccagaggtggccgtgttcgagccttctaaggc
cgagatcgcccacacacaaaaagccaccctcgtgtgcctggccaccggctttttccccgaccacgtggaactgtcttggtgggtcaac
ggcaaagaggtgcactccggcgtgtcaacggatccccagcctctgaaagaacagcctgccctgaacgacagccggtactgcctgag
ctccagactgagagtgtccgccaccttctggcagaacccccggaaccacttcagatgccaggtgcagttttacggcctgagcgagaa
cgacgagtggacccaggacagagccaagcccgtgacacaaatcgtgtctgccgaagcctggggaagagccgattgcggcatcacc
agcgcctcctatcaccagggcgtgctgagcgccacaatcctgtacgaaatcctgctgggcaaggccaccctgtacgccgtgctggtg
tctgctctggtgctgatggccatggtcaagcggaaggactttggcagcggcagagccaaaaggtccgggagcggtGCGACAA
ACTTTAGCCTGTTGAAACAAGCCGGCGACGTTGAAGAGAACCCCGGACCTATGAAGC
TGGTGACCAGCATCACCGTGCTGCTGAGCCTGGGCATCATGGGCGACGCCAAGA
CCACCCAGCCCAACAGCATGGAGAGCAACGAGGAGGAGCCCGTGCACCTGCCCT
GCAACCACAGCACCATCAGCGGCACCGACTACATCCACTGGTACAGGCAGCTG
CCCAGCCAGGGCCCCGAGTACGTGATCCACGGCCTGACCAGCAACGTGAACAA
CAGGATGGCCAGCCTGGCCATCGCCGAGGACAGGAAGAGCAGCACCCTGATCCT
GCACAGGGCCACCCTGAGGGACGCCGCCGTGTACTACTGCATCCTGTTCAACTT
CAACAAGTTCTACTTCGGCAGCGGCACCAAGCTGAACGTGAAGCCCAacatccagaa
ccccgaccccgccgtgtaccagctgagggactccaagtccagcgacaagagcgtgtgtctgtttacggacttcgacagccagaccaa
cgtgagtcaaagcaaggacagcgacgtctacataacggataagaccgtgctggacatgcggagcatggacttcaagagcaacagc
gccgtggcctggtccaacaagagcgacttcgcctgcgccaacgccttcaacaacagcatcatccccgaggacaccttcttccccagc
agcgacgtgccctgcgacgtgaaactggtggagaagtccttcgagacagacaccaatctgaactttcagaacctgctggtgatcgtgc
tgcggattctgctgctgaaagtggccggcttcaatctgctgatgaccctgcggctgtggagc (SEQ ID NO: 32)
Complete Beta and Alpha ORF Protein Sequence (The underlined italic region in the “Furin-
P2A” site allows expression of two polypeptide chains in a single cassette)
MGPGLLHWMALCLLGTGHGDAMVIQNPRYQVTQFGKPVTLSCSQTLNHNVMYWY
QQKSSQAPKLLFHYYDKDFNNEADTPDNFQSRRPNTSFCFLDIRSPGLGDAAMYLCA
TSSGETNEKLFFGSGTQLSVLEdlnkvfppevavfepskaeiahtqkatlvclatgffpdhvelswwvngkevh
sgvstdpqplkeqpalndsryclssrlrvsatfwqnprnhfrcqvqfyglsendewtqdrakpvtqivsaeawgradcgitsasyh
qgvlsatilyeillgkatlyavlvsalvlmamvkrkdfgsgrakrsgsgATNFSLLKQAGDVEENPGPMKLVTSI
TVLLSLGIMGDAKTTQPNSMESNEEEPVHLPCNHSTISGTDYIHWYRQLPSQGPEYVI
HGLTSNVNNRMASLAIAEDRKSSTLILHRATLRDAAVYYCILFNFNKFYFGSGTKL
NVKPNiqnpdpavyqlrdskssdksvclftdfdsqtnvsqskdsdvyitdktvldmrsmdfksnsavawsnksdfacanaf
nnsiipedtffpssdvpcdvklveksfetdtnlnfqnllvivlrilllkvagfnllmtlrlws (SEQ ID NO: 33)
*For certain depicted vectors, MSCV promoter is in bold. Beta chain is annotated using bold and italic text. Alpha chain is annotated using bold and underlined text. CD34-enrichment tag (Q tag) is annotated using italic and underlined text. CD8-alpha is in italic. CD8-beta is underlined.

Included in Tables 1-4 are peptide epitopes, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any sequences listed in Table 1, or a portion thereof. Such polypeptides may have a function of the full-length peptide or polypeptide as described further herein.
Included in Tables 1-4 are RNA nucleic acid molecules (e.g., thymines replaced with uredines), nucleic acid molecules encoding orthologs of the encoded proteins, as well as DNA or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any sequence listed in Tables 1-4, or a portion thereof. Such nucleic acid molecules can have a function of the full-length nucleic acid as described further herein.

In some embodiments, the binding proteins provided herein comprise a constant region that is chimeric, humanized, human, primate, or rodent (e.g., rat or mouse). For example, a human variable region may be chimerized with a murine constant region or a murine variable region may be humanized with a human constant region and/or human framework regions. In some embodiments, the constant regions may be mutated to modify functionality (e.g., introduction of non-naturally occurring cysteine substitutions in opposing residue locations in TCR alpha and beta chains to provide disulfide bonds useful for increasing affinity between the TCR alpha and beta chains). Similarly, mutations may be made in the transmembrane domain of the constant region to modify functionality (e.g., to increase hydrophobicity by introducing a non-naturally occurring substitution of a residue with a hydrophobic amino acid). In some embodiments, each CDR of the binding protein has up to five amino acid substitutions, insertions, deletions, or a combination thereof as compared to a reference CDR sequence. In some embodiments, mutations may be made to the constant region to increase cell surface expression.

In some embodiments, the binding proteins disclosed herein may be engineered protein scaffolds, an antibody or an antigen-binding fragment thereof, TCR-mimic antibodies, and the like. Such binding moieties may be designed and/or generated against peptides and/or MHC-peptide complexes described herein using routine immunological methods, such as immunizing a host, obtaining antibody-producing cells and/or antibodies thereof, and generating hybridomas useful for producing monoclonal antibodies (e.g., Watt et al. (2006) Nat. Biotechnol. 24:177-183; Gebauer and Skerra (2009) Curr. Opin. Chem Biol. 13:245-255; Skerra et al. (2008) FEBS J. 275:2677-2683; Nygren et al. (2008) FEBS J. 275:2668-2676; Dana et al. (2012) Exp. Rev. Mol. Med. 14:e6; Sergeva et al. (2011) Blood 117:4262-4272; PCT Publ. Nos. WO 2007/143104, PCT/US86/02269, and WO 86/01533; U.S. Pat. No. 4,816,567; Better et al. (1988) Science 240:1041-1043; Liu et al. (1987) Proc. Natl. Acad. Sci. U.S.A. 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison, S. L. (1985) Science 229:1202-1207; Oi et al. (1986) Biotechniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060. If desired, binding moieties may be isolated or purified using conventional procedures such as, for example, protein A-Sepharose, hydroxylapatite chromatography, gel electrophoresis, dialysis, affinity chromatography, ammonium sulfate or ethanol precipitation, acid extraction, anion or cation exchange chromatography, phosphocellulose chromatography, hydrophobic interaction chromatography, hydroxylapatite chromatography, lectin chromatography, and high performance liquid chromatography (HPLC) (e.g., Current Protocols in Immunology, or Current Protocols in Protein Science, John Wiley & Sons, NY, N.Y.).

The terms “antibody” and “antibodies” broadly encompass naturally-occurring forms of antibodies (e.g. IgG, IgA, IgM, IgE) and recombinant antibodies, such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

In addition, intrabodies are well-known antigen-binding molecules having the characteristic of antibodies, but that are capable of being expressed within cells in order to bind and/or inhibit intracellular targets of interest (Chen et al. (1994) Human Gene Ther. 5:595-601). Methods are well-known in the art for adapting antibodies to target (e.g., inhibit) intracellular moieties, such as the use of single-chain antibodies (scFvs), modification of immunoglobulin VL domains for hyperstability, modification of antibodies to resist the reducing intracellular environment, generating fusion proteins that increase intracellular stability and/or modulate intracellular localization, and the like. Intracellular antibodies can also be introduced and expressed in one or more cells, tissues or organs of a multicellular organism, for example for prophylactic and/or therapeutic purposes (e.g., as a gene therapy) (see, at least PCT Publ. Nos. WO 08/020079, WO 94/02610, WO 95/22618, and WO 03/014960; U.S. Pat. No. 7,004,940; Cattaneo and Biocca (1997) Intracellular Antibodies: Development and Applications (Landes and Springer-Verlag publs.); Kontermann (2004) Methods 34:163-170; Cohen et al. (1998) Oncogene 17:2445-2456; Auf der Maur et al. (2001) FEBS Lett. 508:407-412; Shaki-Loewenstein et al. (2005) J. Immunol. Meth. 303:19-39).

The term “antibody” as used herein also includes an “antigen-binding portion” of an antibody (or simply “antibody portion”). The term “antigen-binding portion”, as used herein, refers to one or more fragments of an antibody that retain the ability to specifically and/or selectively bind to an antigen (e.g., a peptide and/or an MHC-peptide complex described herein). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16:778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. U.S.A. 90:6444-6448; Poljak et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov et al. (1995) Human Antibodies and Hybridomas 6:93-101) and use of a cysteine residue, protein subunit peptide and a C-terminal polyhistidine tag to make bivalent and biotinylated scFv polypeptides (Kipriyanov et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g. humanized, chimeric, etc.). Antibodies may also be fully human. Preferably, antibodies of the invention bind specifically and/or selectively or substantially specifically and/or selectively to a peptide and/or an MHC-peptide complex described herein. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Similar to other binding moieties described herein, antibodies may also be “humanized,” which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the invention may include amino acid residues not encoded by human germline immunoglobulin sequences (e.g., mutations introduced by random or site-specific mutagenesis in vitrolex vivo or by somatic mutation in vivo), for example in the CDRs. The term “humanized antibody”, as used herein, also includes antibodies in which CDR sequences derived from the germline of another mammalian species, have been grafted onto human framework sequences.

In some embodiments, the binding proteins disclosed herein may comprise a T cell receptor (TCR), an antigen-binding fragment of a TCR, or a chimeric antigen receptor (CAR). In some embodiments, the binding protein disclosed herein may comprise two polypeptide chains, each of which comprises a variable region comprising a CDR3 of a TCR alpha chain and a CDR3 of a TCR beta chain, or a CDR1, CDR2, and CDR3 of both a TCR alpha chain and a TCR beta chain. In some embodiments, a binding protein comprises a single chain TCR (scTCR), which comprises both the TCR V_{α} and TCR V_{β} domains, but only a single TCR constant domain (C_{α} or C_{β}). The term “chimeric antigen receptor” (CAR) refers to a fusion protein that is engineered to contain two or more naturally-occurring amino acid sequences linked together in a way that does not occur naturally or does not occur naturally in a host cell, which fusion protein can function as a receptor when present on a surface of a cell. CARs encompassed by the present invention may include an extracellular portion comprising an antigen-binding domain (i.e., obtained or derived from an immunoglobulin or immunoglobulin-like molecule, such as an antibody or TCR, or an antigen binding domain derived or obtained from a killer immunoreceptor from an NK cell) linked to a transmembrane domain and one or more intracellular signaling domains (optionally containing co-stimulatory domain(s)) (see, e.g., Sadelain et al. (2013) Cancer Discov. 3:388, Harris and Kranz (2016) Trends Pharmacol. Sci. 37:220, and Stone et al. (2014) Cancer Immunol. Immunother. 63:1163).

In some embodiments, 1) the TCR alpha chain CDR, TCR V_{α} domain, and/or TCR alpha chain is encoded by a TRAV, TRAJ, and/or TRAC gene or fragment thereof selected from the group of TRAV, TRAJ, and TRAC genes listed in Table 2, and/or 2) the TCR beta chain CDR, TCR V_{β} domain, and/or TCR beta chain is encoded by a TRBV, TRBJ, and/or TRBC gene or fragment thereof selected from the group of TRBV, TRBJ, and TRBC genes listed in Table 2, and/or 3) each CDR of the binding protein has up to five amino acid substitutions, insertions, deletions, or a combination thereof as compared to the cognate reference CDR sequence listed in Table 2.

In some embodiments, the binding proteins (e.g., the TCR, antigen-binding fragment of a TCR, or chimeric antigen receptor (CAR)) disclosed herein is chimeric (e.g., comprises amino acid residues or motifs from more than one donor or species), humanized (e.g., comprises residues from a non-human organism that are altered or substituted so as to reduce the risk of immunogenicity in a human), or human.

Methods for producing engineered binding proteins, such as TCRs, CARs, and antigen-binding fragments thereof, are well-known in the art (e.g., Bowerman et al. (2009) Mol. Immunol. 5:3000; U.S. Pat. Nos. 6,410,319; 7,446,191; U.S. Pat. Publ. No. 2010/065818; U.S. Pat. No. 8,822,647; PCT Publ. No. WO 2014/031687; U.S. Pat. No. 7,514,537; and Brentjens et al. (2007) Clin. Cancer Res. 73:5426).

In some embodiments, the binding protein described herein is a TCR, or antigen-binding fragment thereof, expressed on a cell surface, wherein the cell surface-expressed TCR is capable of more efficiently associating with a CD3 protein as compared to endogenous TCR. A binding protein encompassed by the present invention, such as a TCR, when expressed on the surface of a cell like a T cell, may also have higher surface expression on the cell as compared to an endogenous binding protein, such as an endogenous TCR. In some embodiments, provided herein is a CAR, wherein the binding domain of the CAR comprises an antigen-specific TCR binding domain (see, e.g., Walseng et al. (2017) Scientific Reports 7:10713).

Also provided are modified binding proteins (e.g., TCRs, antigen-binding fragments of TCRs, or CARs) that may be prepared according to well-known methods using a binding protein having one or more of the V_{α} and/or V_{β} sequences disclosed herein as starting material to engineer a modified binding protein that may have altered properties from the starting binding protein. A binding protein may be engineered by modifying one or more residues within one or both variable regions (i.e., V_{α} and/or V_{β}), for example within one or more CDR regions and/or within one or more framework regions. Additionally or alternatively, a binding protein may be engineered by modifying residues within the constant region(s).

Another type of variable region modification is to mutate amino acid residues within the V_{α} and/or V_{β} CDR1, CDR2 and/or CDR3 regions to thereby improve one or more binding properties (e.g., affinity) of the binding protein of interest. Site-directed mutagenesis or PCR-mediated mutagenesis may be performed to introduce the mutation(s) and the effect on protein binding, or other functional property of interest, may be evaluated in in vitro, ex vivo, or in vivo assays as described herein and provided in the Examples. In some embodiments, conservative modifications (as discussed above) may be introduced. The mutations may be amino acid substitutions, additions or deletions. In some embodiments, the mutations are substitutions. Moreover, typically no more than one, two, three, four or five residues within a CDR region are modified.

In some embodiments, binding proteins (e.g., TCRs, antigen-binding fragments of TCRs, or CARs) described herein may possess one or more amino acid substitutions, deletions, or additions relative to a naturally occurring TCR. In some embodiments, each CDR of the binding protein has up to five amino acid substitutions, insertions, deletions, or a combination thereof as compared to the cognate reference CDR sequence listed in Table 2. Conservative substitutions of amino acids are well-known and may occur naturally or may be introduced when the binding protein is recombinantly produced. Amino acid substitutions, deletions, and additions may be introduced into a protein using mutagenesis methods known in the art (see, e.g., Sambrook et al. (2001) Molecular Cloning: A Laboratory Manual, 3d ed., Cold Spring Harbor Laboratory Press, NY). Oligonucleotide-directed site-specific (or segment specific) mutagenesis procedures may be employed to provide an altered polynucleotide that has particular codons altered according to the substitution, deletion, or insertion desired. Alternatively, random or saturation mutagenesis techniques, such as alanine scanning mutagenesis, error prone polymerase chain reaction mutagenesis, and oligonucleotide-directed mutagenesis may be used to prepare immunogen polypeptide variants (see, e.g., Sambrook et al. supra).

A variety of criteria known to the ordinarily skilled artisan indicate whether an amino acid that is substituted at a particular position in a peptide or polypeptide is conservative (or similar). For example, a similar amino acid or a conservative amino acid substitution is one in which an amino acid residue is replaced with an amino acid residue having a similar side chain. Similar amino acids may be included in the following categories: amino acids with basic side chains (e.g., lysine, arginine, histidine); amino acids with acidic side chains (e.g., aspartic acid, glutamic acid); amino acids with uncharged polar side chains (e.g., glycine, asparagine, glutamine, serine, threonine, tyrosine, cysteine, histidine); amino acids with nonpolar side chains (e.g., alanine, valine, leucine, isoleucine, proline, phenylalanine, methionine, tryptophan); amino acids with beta-branched side chains (e.g., threonine, valine, isoleucine), and amino acids with aromatic side chains (e.g., tyrosine, phenylalanine, tryptophan). Proline, which is considered more difficult to classify, shares properties with amino acids that have aliphatic side chains (e.g., leucine, valine, isoleucine, and alanine). In some embodiments, substitution of glutamine for glutamic acid or asparagine for aspartic acid may be considered a similar substitution in that glutamine and asparagine are amide derivatives of glutamic acid and aspartic acid, respectively. As understood in the art “similarity” between two polypeptides is determined by comparing the amino acid sequence and conserved amino acid substitutes thereto of the polypeptide to the sequence of a second polypeptide (e.g., using GENEWORKS™, Align, the BLAST algorithm, or other algorithms described herein and practiced in the art).

In some embodiments, an encoded binding protein (e.g., TCR, antigen-binding fragment of a TCR, or CAR) may comprise a “signal peptide” (also known as a leader sequence, leader peptide, or transit peptide). Signal peptides target newly synthesized polypeptides to their appropriate location inside or outside the cell. A signal peptide may be removed from the polypeptide during or once localization or secretion is completed. Polypeptides that have a signal peptide are referred to herein as a “pre-protein” and polypeptides having their signal peptide removed are referred to herein as “mature” proteins or polypeptides. In some embodiments, a binding protein (e.g., TCR, antigen-binding fragment of a TCR, or CAR) described herein comprises a mature V_{α} domain, a mature V_{β} domain, or both. In some embodiments, a binding protein (e.g., TCR, antigen-binding fragment of a TCR, or CAR) described herein comprises a mature TCR β-chain, a mature TCR α-chain, or both.

In some embodiments, the binding proteins are fusion proteins comprising: (a) an extracellular component comprising a TCR or antigen-binding fragment thereof; (b) an intracellular component comprising an effector domain or a functional portion thereof; and (c) a transmembrane domain connecting the extracellular and intracellular components. In some embodiments, the fusion protein is capable of binding (e.g., specifically and/or selectively) to a peptide-MHC (pMHC) complex comprising a MAGEA1 immunogenic peptide in the context of an MHC molecule (e.g., an MHC class I molecule). In some embodiments, the MHC molecule comprises an MHC alpha chain that is an HLA serotype HLA-A*02. In some embodiments, the HLA allele is selected from the group consisting of HLA-A*02:01, HLA-A*02:02, HLA-A*02:03, HLA-A*02:05, HLA-A*02:06, and HLA-A*02:07 allele. In specific embodiments, the HLA allele is HLA-A*02:01.

As used herein, an “effector domain” or “immune effector domain” is an intracellular portion or domain of a fusion protein or receptor that can directly or indirectly promote an immune response in a cell when receiving an appropriate signal. In some embodiments, an effector domain is from an immune cell protein or portion thereof or immune cell protein complex that receives a signal when bound (e.g., CD3ζ), or when the immune cell protein or portion thereof or immune cell protein complex binds directly to a target molecule and triggers signal transduction from the effector domain in an immune cell.

An effector domain may directly promote a cellular response when it contains one or more signaling domains or motifs, such as an intracellular tyrosine-based activation motif (ITAM), such as those found in costimulatory molecules. Without wishing to be bound by theory, it is believed that ITAMs are useful for T cell activation following ligand engagement by a T cell receptor or by a fusion protein comprising a T cell effector domain. In some embodiments, the intracellular component or functional portion thereof comprises an ITAM. Exemplary immune effector domains include but are not limited to those from, CD3ε, CD3δ, CD35, CD25, CD79A, CD79B, CARD11, DAP10, FcRα, FcRβ, FcRγ, Fyn, HVEM, ICOS, Lck, LAG3, LAT, LRP, NKG2D, NOTCH1, NOTCH2, NOTCH3, NOTCH4, Wnt, ROR2, Ryk, SLAMF1, Slp76, pTα, TCRα, TCRβ, TRIM, Zap70, PTCH2, or any combination thereof. In some embodiments, an effector domain comprises a lymphocyte receptor signaling domain (e.g., CD3ζ or a functional portion or variant thereof).

In further embodiments, the intracellular component of the fusion protein comprises a costimulatory domain or a functional portion thereof selected from CD27, CD28, 4-1BB (CD137), OX40 (CD134), CD2, CD5, ICAM-1 (CD54), LFA-1 (CD11a/CD18), ICOS (CD278), GITR, CD30, CD40, BAFF-R, HVEM, LIGHT, MKG2C, SLAMF7, NKp80, CD160, B7-H3, a ligand that binds (e.g., specifically and/or selectively) with CD83, or a functional variant thereof, or any combination thereof. In some embodiments, the intracellular component comprises a CD28 costimulatory domain or a functional portion or variant thereof (which may optionally include a LL-GG mutation at positions 186-187 of the native CD28 protein (e.g., Nguyen et al. (2003) Blood 702:4320), a 4-1BB costimulatory domain or a functional portion or variant thereof, or both.

In some embodiments, an effector domain comprises a CD38 endodomain or a functional (e.g., signaling) portion thereof, or a functional variant thereof. In further embodiments, an effector domain comprises a CD27 endodomain or a functional (e.g., signaling) portion thereof, or a functional variant thereof. In further embodiments, an effector domain comprises a CD28 endodomain or a functional (e.g., signaling) portion thereof, or a functional variant thereof. In still further embodiments, an effector domain comprises a 4-1BB endodomain or a functional (e.g., signaling) portion thereof, or a functional variant thereof. In further embodiments, an effector domain comprises an OX40 endodomain or a functional (e.g., signaling) portion thereof, or a functional variant thereof. In further embodiments, an effector domain comprises a CD2 endodomain or a functional (e.g., signaling) portion thereof, or a functional variant thereof. In further embodiments, an effector domain comprises a CD5 endodomain or a functional (e.g., signaling) portion thereof, or a functional variant thereof. In further embodiments, an effector domain comprises an ICAM-1 endodomain or a functional (e.g., signaling) portion thereof, or a functional variant thereof. In further embodiments, an effector domain comprises a LFA-1 endodomain or a functional (e.g., signaling) portion thereof, or a functional variant thereof. In further embodiments, an effector domain comprises an ICOS endodomain or a functional (e.g., signaling) portion thereof, or a functional variant thereof.

An extracellular component and an intracellular component encompassed by the present invention are connected by a transmembrane domain. A “transmembrane domain,” as used herein, is a portion of a transmembrane protein that can insert into or span a cell membrane. Transmembrane domains have a three-dimensional structure that is thermodynamically stable in a cell membrane and generally range in length from about 15 amino acids to about 30 amino acids. The structure of a transmembrane domain may comprise an alpha helix, a beta barrel, a beta sheet, a beta helix, or any combination thereof. In some embodiments, the transmembrane domain comprises or is derived from a known transmembrane protein (e.g., a CD4 transmembrane domain, a CD8 transmembrane domain, a CD27 transmembrane domain, a CD28 transmembrane domain, or any combination thereof).

In some embodiments, the extracellular component of the fusion protein further comprises a linker disposed between the binding domain and the transmembrane domain. As used herein when referring to a component of a fusion protein that connects the binding and transmembrane domains, a “linker” may be an amino acid sequence having from about two amino acids to about 500 amino acids, which can provide flexibility and room for conformational movement between two regions, domains, motifs, fragments, or modules connected by the linker. For example, a linker encompassed by the present invention can position the binding domain away from the surface of a host cell expressing the fusion protein to enable proper contact between the host cell and a target cell, antigen binding, and activation (Patel et al. (1999) Gene Therapy 6:412-419). Linker length may be varied to maximize antigen recognition based on the selected target molecule, selected binding epitope, or antigen binding domain seize and affinity (see, e.g., Guest et al. (2005) Immunother. 28:203-11 and PCT Publ. No. WO 2014/031687). Exemplary linkers include those having a glycine-serine amino acid chain having from one to about ten repeats of GlyₓSer_{y} (SEQ ID NO: 34), wherein x and y are each independently an integer from 0 to 10, provided that x and y are not both 0 (e.g., (Gly₄Ser)₂ (SEQ ID NO: 35), (Gly₃Ser)₂ (SEQ ID NO: 36), Gly₂Ser, or a combination thereof, such as ((Gly₃Ser)₂Gly₂Ser) (SEQ ID NO: 37)).

A binding protein may be conjugated to an agent, such as a detection moiety, readiosensitizer, photosensitizer, and the like, and/or may be chemically modified as described above regarding peptides.

Binding proteins encompassed by the present invention may, in some embodiments, be covalently linked to a moiety. In some embodiments, the covalently linked moiety comprises an affinity tag or a label. The affinity tag may be selected from the group consisting of Glutathione-S-Transferase (GST), calmodulin binding protein (CBP), protein C tag, Myc tag, HaloTag, HA tag, Flag tag, His tag, biotin tag, and V5 tag. The label may be a fluorescent protein. In some embodiments, the covalently linked moiety is selected from the group consisting of an inflammatory agent, an anti-inflammatory agent, a cytokine, a toxin, a cytotoxic molecule, a radioactive isotope, or an antibody such as a single-chain Fv.

A binding protein may be conjugated to an agent used in imaging, research, therapeutics, theranostics, pharmaceuticals, chemotherapy, chelation therapy, targeted drug delivery, and radiotherapy. In some embodiments, a binding protein may be conjugated to or fused with detectable agents, such as a fluorophore, a near-infrared dye, a contrast agent, a nanoparticle, a metal-containing nanoparticle, a metal chelate, an X-ray contrast agent, a PET agent, a metal, a radioisotope, a dye, radionuclide chelator, or another suitable material that can be used in imaging. In some embodiments, 1, 2, 3, 4, 5, 6, 7, 8, 9, 10, or more detectable moieties may be linked to a binding protein. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212. In some embodiments, the near-infrared dyes are not easily quenched by biological tissues and fluids. In some embodiments, the fluorophore is a fluorescent agent emitting electromagnetic radiation at a wavelength between 650 nm and 4000 nm, such emissions being used to detect such agent. Non-limiting examples of fluorescent dyes that may be used as a conjugating molecule include DyLight-680, DyLight-750, VivoTag-750, DyLight-800, IRDye-800, VivoTag-680, Cy5.5, ZQ800, or indocyanine green (ICG). In some embodiments, near infrared dyes often include cyanine dyes (e.g., Cy7, Cy5.5, and Cy5). Additional, non-limiting examples of fluorescent dyes for use as a conjugating molecule in accordance with present invention include acradine orange or yellow, Alexa Fluors® (e.g., Alexa Fluor® 790, 750, 700, 680, 660, and 647) and any derivative thereof, 7-actinomycin D, 8-anilinonaphthalene-1-sulfonic acid, ATTO® dye and any derivative thereof, auramine-rhodamine stain and any derivative thereof, bensantrhone, bimane, 9-10-bis(phenylethynyl) anthracene, 5,12-bis(phenylethynyl) naththacene, bisbenzimide, brainbow, calcein, carbodyfluorescein and any derivative thereof, 1-chloro-9,10-bis(phenylethynyl) anthracene and any derivative thereof, DAPI, DiOC6, DyLight® Fluors® and any derivative thereof, epicocconone, ethidium bromide, FLASH-EDT2®, Fluo dye and any derivative thereof, FluoProbe® and any derivative thereof, fluorescein and any derivative thereof, Fura® and any derivative thereof, GelGreen® and any derivative thereof, GelRed® and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluorescent protein and YOYO-1. Other suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green™ dyes (e.g., Oregon Green™ 488, 500, 514, etc.), Texas Red®, Texas Red®-X, SPECTRUM RED®, SPECTRUM GREEN®, cyanine dyes (e.g., CY-3, Cy-5, CY-3.5, CY-5.5, etc.), Alexa Fluor® dyes (e.g., Alexa Fluor® 350, 488, 532, 546, 568, 594, 633, 660, 680, etc.), BODIPY® dyes (e.g., BODIPY® FL, R6G, TMR, TR, 530/550, 558/568, 564/570, 576/589, 581/591, 630/650, 650/665, etc.), IRD dyes (e.g., IRD40™, IRD700™, IRD800™, etc.), and the like. Additional suitable detectable agents are well-known in the art (e.g., PCT Publ. No. PCT/US14/56177). Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212.

Binding proteins may be conjugated to a radiosensitizer or photosensitizer. Examples of radiosensitizers include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers include but are not limited to: fluorescent molecules or beads that generate heat when illuminated, nanoparticles, porphyrins and porphyrin derivatives (e.g., chlorins, bacteriochlorins, isobacteriochlorins, phthalocyanines, and naphthalocyanines), metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach allows for highly specific targeting of cells of interest (e.g., immune cells) using both a therapeutic agent (e.g., drug) and electromagnetic energy (e.g., radiation or light) concurrently. In some embodiments, the binding protein is fused with, or covalently or non-covalently linked to the agent, for example, directly or via a linker.

In some embodiments, the binding protein may be chemically modified. For example, a binding protein may be mutated to modify peptide properties such as detectability, stability, biodistribution, pharmacokinetics, half-life, surface charge, hydrophobicity, conjugation sites, pH, function, and the like. N-methylation is one example of methylation that can occur in a binding protein encompassed by the present invention. In some embodiments, a binding protein may be modified by methylation on free amines such as by reductive methylation with formaldehyde and sodium cyanoborohydride.

A chemical modification may comprise a polymer, a polyether, polyethylene glycol, a biopolymer, a zwitterionic polymer, a polyamino acid, a fatty acid, a dendrimer, an Fc region, a simple saturated carbon chain such as palmitate or myristolate, or albumin. The chemical modification of a binding protein with an Fc region may be a fusion Fc-protein. A polyamino acid may include, for example, a poly amino acid sequence with repeated single amino acids (e.g., poly glycine), and a poly amino acid sequence with mixed poly amino acid sequences that may or may not follow a pattern, or any combination of the foregoing.

In some embodiments, the binding proteins encompassed by the present invention may be modified. In some embodiments, the modifications having substantial or significant sequence identity to a parent binding protein to generate a functional variant that maintains one or more biophysical and/or biological activities of the parent binding protein (e.g., maintain pMHC binding specificity). In some embodiments, the mutation is a conservative amino acid substitution.

In some embodiments, binding proteins encompassed by the present invention may comprise synthetic amino acids in place of one or more naturally-occurring amino acids. Such synthetic amino acids are well-known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, a-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, oc-aminocyclohexane carboxylic acid, α-aminocycloheptane carboxylic acid, a-(2-amino-2-norbornane)-carboxylic acid, α,γ-diaminobutyric acid, β-diaminopropionic acid, homophenylalanine, and oc-tert-butylglycine.

Binding proteins encompassed by the present invention may be glycosylated, amidated, carboxylated, phosphorylated, esterified, N-acylated, cyclized (e.g., via a disulfide bridge), or converted into an acid addition salt and/or optionally dimerized or polymerized, or conjugated.

In some embodiments, the attachment of a hydrophobic moiety, such as to the N-terminus, the C-terminus, or an internal amino acid, may be used to extend half-life of a peptide encompassed by the present invention. In other embodiments, a binding protein may include post-translational modifications (e.g., methylation and/or amidation), which can affect, for example, serum half-life. In some embodiments, simple carbon chains (e.g., by myristoylation and/or palmitylation) may be conjugated to the binding proteins. In some embodiments, the simple carbon chains may render the binding proteins easily separable from the unconjugated material. For example, methods that may be used to separate the binding proteins from the unconjugated material include, but are not limited to, solvent extraction and reverse phase chromatography. The lipophilic moieties can extend half-life through reversible binding to serum albumin. The conjugated moieties may be lipophilic moieties that extend half-life of the peptides through reversible binding to serum albumin. In some embodiments, the lipophilic moiety may be cholesterol or a cholesterol derivative, including cholestenes, cholestanes, cholestadienes and oxysterols. In some embodiments, the binding proteins may be conjugated to myristic acid (tetradecanoic acid) or a derivative thereof. In other embodiments, a binding protein may be coupled (e.g., conjugated) to a half-life modifying agent. Examples of half-life modifying agents include but are not limited to: a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, or a molecule that binds to albumin. In some embodiments, a spacer or linker may be coupled to a binding protein, such as 1, 2, 3, 4, or more amino acid residues that serve as a spacer or linker in order to facilitate conjugation or fusion to another molecule, as well as to facilitate cleavage of the peptide from such conjugated or fused molecules. In some embodiments, binding proteins may be conjugated to other moieties that, for example, can modify or effect changes to the properties of the binding proteins.

A binding protein may be produced recombinantly or synthetically, such as by solid-phase peptide synthesis or solution-phase peptide synthesis. Polypeptide synthesis may be performed by known synthetic methods, such as using fluorenylmethyloxycarbonyl (Fmoc) chemistry or by butyloxycarbonyl (Boc) chemistry. Polypeptide fragments may be joined together enzymatically or synthetically.

In an aspect encompassed by the present invention, provided herein are methods of producing a binding protein described herein, comprising the steps of: (i) culturing a transformed host cell which has been transformed by a nucleic acid comprising a sequence encoding a binding protein described herein under conditions suitable to allow expression of said binding protein; and (ii) recovering the expressed binding protein.

Methods useful for isolating and purifying recombinantly produced binding protein, by way of example, may include obtaining supernatants from suitable host cell/vector systems that secrete the binding protein into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of binding proteins described herein include batch cell culture, which is monitored and controlled to maintain appropriate culture conditions. Purification of the binding protein may be performed according to methods described herein and known in the art. In any of the herein disclosed embodiments, the encoded binding protein is capable of bind to a peptide-MHC (pMHC) complex comprising a MAGEA1 immunogenic peptide in the context of an MHC molecule (e.g., an MHC class I molecule). In some embodiments, the MHC molecule comprises an MHC alpha chain that is an HLA serotype HLA-A*02. In some embodiments, the HLA allele is selected from the group consisting of HLA-A*02:01, HLA-A*02:02, HLA-A*02:03, HLA-A*02:05, HLA-A*02:06, and HLA-A*02:07 allele. In specific embodiments, the HLA allele is HLA-A*02:01.

A variety of assays are well-known for assessing binding affinity and/or determining whether a binding molecule binds (e.g., specifically and/or selectively) to a particular ligand (e.g., peptide antigen-MHC complex). It is within the level of a skilled artisan to determine the binding affinity of a binding protein for a target, such as a T cell peptide epitope of a target polypeptide, such as by using any of a number of binding assays that are well-known in the art. For example, in some embodiments, a Biacore™ machine may be used to determine the binding constant of a complex between two proteins. The dissociation constant (K_{D}) for the complex may be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoas says (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy and surface plasmon resonance (Biacore™) analysis (see, e.g., Scatchard et al. (1949) Ann. N.Y. Acad. Sci. 51:660, Wilson (2002) Science 295:2103, Wolff et al. (1993) Cancer Res. 53:2560, and U.S. Pat. Nos. 5,283,173 and 5,468,614), flow cytometry, sequencing and other methods for detection of expressed nucleic acids. In one example, apparent affinity for a target is measured by assessing binding to various concentrations of tetramers, for example, by flow cytometry using labeled multimers, such as MHC-antigen tetramers. In one representative example, apparent K_{D} of a binding protein is measured using 2-fold dilutions of labeled tetramers at a range of concentrations, followed by determination of binding curves by non-linear regression, apparent K_{D} being determined as the concentration of ligand that yielded half-maximal binding.

### VI. Nucleic Acids and Vectors

In an aspect encompassed by the present invention, provided herein are nucleic acid molecules that encode proteins described herein, such as MAGEA1 immunogenic peptides and fragments thereof, MHC molecules, binding proteins (e.g., TCRs, antigen-binding fragments of the TCRs, CARs, and the like), and the like.

In some embodiments, the nucleic acid molecule hybridizes, under stringent conditions, with the complement of a sequence with at least about at least about 80%, 81%, 82%, 83%, 84%, 85%, 86%, 87%, 88%, 89%, 90%, 91%, 92%, 93%, 94%, 95%, 96%, 97%, 98%, 99%, or more identity, such as over the full length, to a nucleic acid encoding a polypeptide selected from the group consisting of the polypeptide sequences listed in Tables 1-3.

In some embodiments, the nucleic acid molecule hybridizes, under stringent conditions, with the complement of a nucleic acid encoding a polypeptide selected from the group consisting of polypeptide sequences listed in Tables 1-3.

In some embodiments, the nucleic acid molecule comprises (e.g., comprises, consists essentially of, or consists of) a nucleotide sequence encoding a polypeptide selected from the group consisting of polypeptide sequences listed in Tables 1-3.

In some embodiments, the nucleic acid sequence encodes a MAGEA1 immunogenic peptides described herein.

In some embodiments, the nucleic acids comprise (e.g., comprise, consist essentially of, or consist of) a nucleotide sequence encoding at least one (e.g., one, two, or three) TCR α-chain CDR set forth in Table 2. In some embodiments, the nucleic acids comprise (e.g., comprise, consist essentially of, or consist of) a nucleotide sequence encoding a TCR V_{α} domain having an amino acid sequence that is at least about at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR V_{α} domain sequence set forth in Table 2. In some embodiments, the nucleic acids comprise (e.g., comprise, consist essentially of, or consist of) a nucleotide sequence encoding a TCR α-chain having an amino acid sequence that is at least about at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR α-chain sequence set forth in Table 2.

In some embodiments, the nucleic acids comprise (e.g., comprise, consist essentially of, or consist of) a nucleotide sequence encoding at least one (e.g., one, two, or three) TCR β-chain CDR set forth in Table 2. In some embodiments, the nucleic acids comprise (e.g., comprise, consist essentially of, or consist of) a nucleotide sequence encoding a TCR V_{β} domain having an amino acid sequence that is at least about at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR V_{β} domain sequence set forth in Table 2. In some embodiments, the nucleic acids comprise (e.g., comprise, consist essentially of, or consist of) a nucleotide sequence encoding a TCR β-chain having an amino acid sequence that is at least about at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR β-chain sequence set forth in Table 2.

The term “nucleic acid” includes “polynucleotide,” “oligonucleotide,” and “nucleic acid molecule,” and generally means a polymer of DNA or RNA, which may be single-stranded or double-stranded, synthesized or obtained (e.g., isolated and/or purified) from natural sources, which may contain natural, non-natural or altered nucleotides, and which may contain a natural, non-natural or altered internucleotide linkage, such as a phosphoroamidate linkage or a phosphorothioate linkage, instead of the phosphodiester found between the nucleotides of an unmodified oligonucleotide. In an embodiment, the nucleic acid comprises complementary DNA (cDNA).

In some embodiments, the nucleic acids encompassed by the present invention are recombinant. As used herein, the term “recombinant” refers to (i) molecules that are constructed outside living cells by joining natural or synthetic nucleic acid segments to nucleic acid molecules that may replicate in a living cell, or (ii) molecules that result from the replication of those described in (i) above. For purposes herein, the replication may be in vitro, ex vivo, or in vivo replication.

The nucleic acids can be constructed based on chemical synthesis and/or enzymatic ligation reactions using procedures known in the art. See, for example, Green and Sambrook et al. supra. For example, a nucleic acid may be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that may be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids encompassed by the present invention can be purchased from companies, such as Integrated DNA Technologies (Coralville, IA).

In one embodiment, the nucleic acid comprises a codon-optimized nucleotide sequence. Without being bound to a particular theory or mechanism, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency. In some embodiments, the nucleotide sequences described herein are codon-optimized for expression in a host cell (e.g., an immune cell, such as a T cell).

The present invention also provides a nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions may hybridize under high stringency conditions. By “high stringency conditions” is meant that the nucleotide sequence specifically and/or selectively hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive TCRs. It is generally appreciated that conditions may be rendered more stringent by the addition of increasing amounts of formamide.

The present invention also provides a nucleic acid comprising a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, 99%, or more identical to any of the nucleic acids described herein.

Typically, said nucleic acid is a DNA or RNA molecule, which may be included in a suitable vector, such as a plasmid, cosmid, episome, artificial chromosome, phage or a viral vector.

The terms “vector”, “cloning vector” and “expression vector” mean the vehicle by which a DNA or RNA sequence (e.g., a foreign gene) can be introduced into a host cell, so as to transform the host and promote expression (e.g., transcription and translation) of the introduced sequence. Thus, a further object encompassed by the present invention relates to a vector comprising a nucleic acid encompassed by the present invention.

Such vectors may comprise regulatory elements, such as a promoter, enhancer, terminator and the like, to cause or direct expression of said polypeptide upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like.

Any expression vector for animal cell may be used. Examples of suitable vectors include pAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like. Other representative examples of plasmids include replicating plasmids comprising an origin of replication, or integrative plasmids, such as for instance pUC, pcDNA, pBR, and the like. Representative examples of viral vector include adenoviral, retroviral, lentiviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv-positive cells, 293 cells, etc. Detailed protocols for producing such replication-defective recombinant viruses are well-known in the art and may be found, for instance, in PCT Publ. WO 95/14785, PCT. Publ. WO 96/22378, U.S. Pat. Nos. 5,882,877, 6,013,516, 4,861,719, 5,278,056, and PCT Publ. WO 94/19478.

In some embodiments, the composition comprises an expression vector comprising an open reading frame encoding a binding protein or a polypeptide described herein or a fragment thereof. In some embodiments, the nucleic acid includes regulatory elements necessary for expression of the open reading frame. Such elements may include, for example, a promoter, an initiation codon, a stop codon, and a polyadenylation signal. In addition, enhancers may be included. These elements may be operably linked to a sequence that encodes the binding protein, polypeptide or fragment thereof.

In some embodiments, the vector further comprises nucleic acid sequence encoding CD8α, CD8β, a dominant negative TGFβ receptor (e.g., a DN-TGFβRII), selectable protein marker, optionally wherein the selectable protein marker is dihydrofolate reductase (DHFR). In certain embodiments, the nucleic acid sequence encoding CD8α, CD8β, the DN-TGFβR, and/or the selectable protein marker is operably linked to a nucleic acid encoding a tag (e.g., a CD34 enrichment tag). In specific embodiments, a nucleic acid sequence described herein, such as a nucleic acid sequence encoding a TCRα, TCRβ. CD8α, CD8β, DN-TGFβR, and/or selectable protein marker are interconnected with an internal ribosome entry site or a nucleic acid sequence encoding a self-cleaving peptide, such as P2A, E2A, F2A or T2A, etc.

In some embodiments, the expression vector provided herein comprises a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, 99%, or more identical to any of the nucleic acids set forth in Tables 1-3.

As described above, representative examples of promoters include, but are not limited to, promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, Cytomegalovirus (CMV) such as the CMV immediate early promoter, Epstein Barr Virus (EBV), Rous Sarcoma Virus (RSV) as well as promoters from human genes such as human actin, human myosin, human hemoglobin, human muscle creatine, and human metalothionein. Examples of suitable polyadenylation signals include but are not limited to SV40 polyadenylation signals and LTR polyadenylation signals.

In addition to the regulatory elements required for expression, other elements may also be included in the nucleic acid molecule. Such additional elements include enhancers. Enhancers include the promoters described herein. In some embodiments, enhancers/promoters include, for example, human actin, human myosin, human hemoglobin, human muscle creatine and viral enhancers such as those from CMV, RSV and EBV.

In some embodiments, the nucleic acid may be operably incorporated in a carrier or delivery vector as described further below. Useful delivery vectors include but are not limited to biodegradable microcapsules, immuno-stimulating complexes (ISCOMs) or liposomes, and genetically engineered attenuated live carriers such as viruses or bacteria.

In some embodiments, the vector is a viral vector, such as lentiviruses, retroviruses, herpes viruses, adenoviruses, adeno-associated viruses, vaccinia viruses, baculoviruses, Fowl pox, AV-pox, modified vaccinia Ankara (MVA) and other recombinant viruses. For example, a lentivirus vector may be used to infect T cells.

In some embodiments, the recombinant expression vector is capable of delivering a polynucleotide to an appropriate host cell, for example, a T cell or an antigen-presenting cell, i.e., a cell that displays a peptide/MHC complex on its cell surface (e.g., a dendritic cell) and lacks CD8. In some embodiments, the host cell is a hematopoietic progenitor cell or a human immune system cell. For example, the immune system cell may be a CD4⁺ T cell, a CD8⁺ T cell, a CD4/CD8 double negative T cell, a gd T cell, a natural killer cell, a dendritic cell, or any combination thereof. In some embodiments, wherein a T cell is the host, the T cell may be naive, a central memory T cell, an effector memory T cell, or any combination thereof. The recombinant expression vectors may therefore also include, for example, lymphoid tissue-specific transcriptional regulatory elements (TREs), such as a B lymphocyte, T lymphocyte, or dendritic cell specific TREs. Lymphoid tissue specific TREs are known in the art (see, e.g., Thompson et al. (1992) Mol. Cell. Biol. 72:1043, Todd et al. (1993) J. Exp. Med. 777:1663, and Penix et al. (1993) J. Exp. Med. 775:1483).

In some embodiments, a recombinant expression vector comprises a nucleotide sequence encoding a TCR α chain, a TCR β chain, and/or a linker peptide. For example, in some embodiments, the recombinant expression vector comprises a nucleotide sequence encoding the full-length TCR alpha and TCR beta chains of the binding protein with a linker positioned between them, wherein the nucleotide sequence encoding the beta chain is positioned 5′ of the nucleotide sequence encoding the alpha chain. In some embodiments, the nucleotide sequence encodes the full-length TCR alpha and TCR beta chains with a linker positioned between them, wherein the nucleotide sequence encoding the TCR beta chain is positioned 3′ of the nucleotide sequence encoding the TCR alpha chain. In some embodiments, the full-length TCR alpha and/or TCR beta chains are replaced with fragments thereof.

As described further below, another aspect encompassed by the present invention relates to a cell which has been transfected, infected or transformed by a nucleic acid and/or a vector in accordance with the present invention. A host cell may include any individual cell or cell culture which may receive a vector or the incorporation of nucleic acids and/or proteins, as well as any progeny cells. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods (see, e.g., Sambrook el al. (1989) Molecular Cloning: A Laboratory Manual 2d ed. (Cold Spring Harbor Laboratory)). The term “transformation” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed.”

The nucleic acids encompassed by the present invention may be used to produce a recombinant polypeptide encompassed by the present invention in a suitable expression system. The term “expression system” means a host cell and compatible vector under suitable conditions, e.g., for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al (1980), rat YB2/3HL.P2.G11.16Ag.20 cell (ATCC CRL 1662, hereinafter referred to as “YB2/0 cell”), and the like. In some embodiments, the YB2/0 cell is used since ADCC activity of chimeric or humanized binding proteins is enhanced when expressed in this cell.

The present invention also encompasses methods of producing a recombinant host cell expressing binding proteins, peptides and fragments thereof encompassed by the present invention, said method comprising the steps consisting of (i) introducing in vitro or ex vivo a recombinant nucleic acid or a vector as described above into a competent host cell, (ii) culturing in vitro or ex vivo the recombinant host cell obtained and (iii), optionally, selecting the cells which express said binding proteins, peptides and fragments thereof. Such recombinant host cells may be used for the diagnostic, prognostic, and/or therapeutic method encompassed by the present invention.

In another aspect, as described above, the present invention provides isolated nucleic acids that hybridize under selective hybridization conditions to a polynucleotide disclosed herein. Thus, the polynucleotides of this embodiment may be used for isolating, detecting, and/or quantifying nucleic acids comprising such polynucleotides. For example, polynucleotides encompassed by the present invention may be used to identify, isolate, or amplify partial or full-length clones in a deposited library. In some embodiments, the polynucleotides are genomic or cDNA sequences isolated, or otherwise complementary to, a cDNA from a human or mammalian nucleic acid library. In some embodiments, the cDNA library comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or any range in between, inclusive, such as at least about 80%-100%, full-length sequences. The cDNA libraries may be normalized to increase the representation of rare sequences. Low or moderate stringency hybridization conditions are typically, but not exclusively, employed with sequences having a reduced sequence identity relative to complementary sequences. Moderate and high stringency conditions may optionally be employed for sequences of greater identity. Low stringency conditions allow selective hybridization of sequences having about 70% sequence identity and may be employed to identify orthologous or paralogous sequences. Optionally, polynucleotides encompassed by the present invention will encode at least a portion of a binding protein encoded by the polynucleotides described herein. The polynucleotides encompassed by the present invention embrace nucleic acid sequences that may be employed for selective hybridization to a polynucleotide encoding a binding protein encompassed by the present invention (see, e.g., Ausubel, supra and Colligan, supra).

### VII. Engineered Cells

In an aspect encompassed by the present invention, provided herein are host cells that express proteins described herein, such as MAGEA1 immunogenic peptides, MAGEA1 immunogenic peptide-MHC (pMHC) complexes, MAGEA1 binding proteins (e.g., TCRs, antigen-binding fragments of TCRs, CARs, or fusion proteins comprising a TCR and an effector domain), and the like described herein. In some embodiments, the host cells comprise the nucleic acids or vectors described herein.

In some embodiments, a polynucleotide encoding a binding protein is used to transform, transfect, or transduce a host cell (e.g., a T cell) for use in adoptive transfer therapy. Advances in nucleic acid sequencing and particular TCR sequencing have been described (e.g., Robins et al. (2009) Blood 114:4099; Robins et al. (2010) Sci. Translat. Med. 2:47ra64, Robins et al. (2011) J. Imm. Meth., and Warren et al. (2011) Genome Res. 21:790) and may be employed in the course of practicing embodiments encompassed by the present invention. Similarly, methods for transfecting or transducing T cells with desired nucleic acids are well-known in the art (e.g., U.S. Pat. Publ. No. US 2004/0087025) as have adoptive transfer procedures using T cells of desired antigen-specificity (e.g., Schmitt et al. (2009) Hum. Gen. 20:1240, Dossett et al. (2009) Mol. Ther. 77:742, Till et al. (2008) Blood 772:2261, Wang et al. (2007) Hum. Gene Ther. 18:112, Kuball et al. (2007) Blood 709:2331, U.S. Pat. Publ. 2011/0243972, U.S. Pat. Publ. 2011/0189141, and Leen et al. (2007) Ann. Rev. Immunol. 25:243).

Any suitable immune cell may be modified to include a heterologous polynucleotide encompassed by the present invention, including, for example, a T cell, a NK cell, or a NK-T cell. In some embodiments, the cell may be a primary cell or a cell of a cell line. In some embodiments, a modified immune cell comprises a CD4⁺ T cell, a CD8⁺ T cell, or both. For purposes herein, the T cell may be any T cell, such as a cultured T cell, e.g., a primary T cell, or a T cell from a cultured T cell line, e.g., Jurkat, SupT1, etc., or a T cell obtained from a mammal. If obtained from a mammal, the T cell may be obtained from numerous sources, including but not limited to blood, bone marrow, lymph node, the thymus, or other tissues or fluids. T cells may also be enriched for or purified. In some embodiments, the T cell is a human T cell. In some embodiments, the T cell is a T cell isolated from a human. The T cell may be any type of T cell and may be of any developmental stage, including but not limited to, cytotoxic lymphocyte, cytotoxic lymphocyte precursor cell, cytotoxic lymphocyte progenitor cell, cytotoxic lymphocyte stem cell, CD4⁺/CD8⁺ double positive T cells, CD4⁺ helper T cells, e.g., Th1 and Th2 cells, CD4⁺ T cells, CD8⁺ T cells (e.g., cytotoxic T cells), tumor infiltrating lymphocytes (TILs), memory T cells (e.g., central memory T cells and effector memory T cells), naive T cells, and the like.

Any appropriate method may be used to transfect or transduce the cells (e.g., T cells), or to administer the nucleotide sequences or compositions encompassed by methods described herein. Methods for delivering polynucleotides to host cells include, for example, use of cationic polymers, lipid-like molecules, and certain commercial products such as, for example, in vivo-jetPEI®. Other methods include ex vivo transduction, injection, electroporation, DEAE-dextran, sonication loading, liposome-mediated transfection, receptor-mediated transduction, microprojectile bombardment, transposon-mediated transfer, and the like. Still further methods of transfecting or transducing host cells employ vectors, described in further detail herein.

Modified immune cells as described herein may be functionally characterized using methodologies for assaying T cell activity, including determination of T cell binding, activation or induction and also including determination of T cell responses that are antigen-specific. Examples include determination of T cell proliferation, T cell cytokine release, antigen-specific T cell stimulation, MHC restricted T cell stimulation, CTL activity (e.g., by detecting ⁵¹Cr release from pre-loaded target cells), changes in T cell phenotypic marker expression, and other measures of T-cell functions.

Procedures for performing these and similar assays may be found, for example, in Lefkovits (Immunology Methods Manual: Hie Comprehensive Sourcebook of Techniques, 1998), as well as Current Protocols in Immunology, Weir, (1986) Handbook of Experimental Immunology, Blackwell Scientific, Boston, MA; Mishell and Shigii (eds.) (1979) Selected Methods in Cellular Immunology, Freeman Publishing, San Francisco, CA; Green and Reed (1998) Science 281:1309, and references cited therein.

In some embodiments, apparent affinity for a binding protein, such as a TCR or antigen-binding portion thereof, may be measured by assessing binding to various concentrations of MHC multimers. “MHC-peptide multimer staining” refers to an assay used to detect antigen-specific T cells, which, in some embodiments, features a tetramer of MHC molecules, each comprising an identical peptide having an amino acid sequence that is cognate (e.g., identical or related to) at least one antigen (e.g., a MAGEA1 immunogenic peptide), wherein the complex is capable of binding to a binding protein, such as a TCR or antigen-binding portion thereof, that recognizes the cognate antigen. Each of the MHC molecules may be tagged with a biotin molecule. Biotinylated MHC/peptides may be multimerized (e.g., tetramerized) by the addition of streptavidin, which may be fluorescently labeled.

The multimer may be detected by flow cytometry via the fluorescent label. In some embodiments, a pMHC multimer assay is used to detect or select enhanced affinity binding protein, such as a TCR or antigen-binding portion thereof, encompassed by the present invention. In some examples, apparent K_{D} of a binding protein, such as a TCR or antigen-binding portion thereof, is measured using 2-fold dilutions of labeled multimers at a range of concentrations, followed by determination of binding curves by non-linear regression, apparent K_{D} being determined as the concentration of ligand that yielded half-maximal binding.

Levels of cytokines may be determined using methods described herein, such as ELISA, ELISPOT, intracellular cytokine staining, and flow cytometry and combinations thereof (e.g., intracellular cytokine staining and flow cytometry).

Immune cell proliferation and clonal expansion resulting from an antigen-specific elicitation or stimulation of an immune response may be determined by isolating lymphocytes, such as circulating lymphocytes in samples of peripheral blood cells or cells from lymph nodes, stimulating the cells with antigen, and measuring cytokine production, cell proliferation and/or cell viability, such as by incorporation of tritiated thymidine or non-radioactive assays, such as MTT assays and the like. The effect of an immunogen described herein on the balance between a Th1 immune response and a Th2 immune response may be examined, for example, by determining levels of Th1 cytokines, such as IFN-g, IL-12, IL-2, and TNF-b, and Type 2 cytokines, such as IL-4, IL-5, IL-9, IL-10, and IL-13.

A host cell encompassed by the present invention may comprise a single polynucleotide that encodes a binding protein as described herein, or the binding protein may be encoded by more than one polynucleotide. In other words, components or portions of a binding protein may be encoded by two or more polynucleotides, which may be contained on a single nucleic acid molecule or may be contained on two or more nucleic acid molecules.

Moreover, as described further below and in the working examples, a host ell encompassed by the present invention may encode and/or express useful accessory proteins in addition to a binding protein as described herein, either on the same polynucleotide or a different polynucleotide as the binding protein or components thereof. For example, the host cell may encode and/or express CD8α, CD8β, a DN-TGFβR (e.g., a DN-TGFβRII), and/or a selectable protein marker, optionally wherein the selectable protein marker is DHFR.

In some embodiments, a polynucleotide encoding two or more components or portions of a binding protein encompassed by the present invention comprises the two or more coding sequences operatively associated in a single open reading frame. Such an arrangement can advantageously allow coordinated expression of desired gene products, such as, for example, contemporaneous expression of alpha- and beta-chains of a TCR, such that they are produced in about a 1:1 ratio. In some embodiments, two or more substituent gene products of a binding protein encompassed by the present invention, such as a TCR (e.g., alpha- and beta-chains) or CAR, are expressed as separate molecules and associate post-translationally. In further embodiments, two or more substituent gene products of a binding protein encompassed by the present invention are expressed as a single peptide with the parts separated by a cleavable or removable segment. For instance, self-cleaving peptides useful for expression of separable polypeptides encoded by a single polynucleotide or vector are known in the art and include, for example, a porcine teschovirus-1 2 A (P2A) peptide, a those asigna virus 2A (T2A) peptide, an equine rhinitis A virus (ERAV) 2A (E2A) peptide, and a foot-and-mouth disease vims 2A (F2A) peptide.

In some embodiments, a binding protein encompassed by the present invention comprises one or more junction amino acids. “Junction amino acids” or “junction amino acid residues” refer to one or more (e.g., 2 to about 10) amino acid residues between two adjacent motifs, regions or domains of a polypeptide, such as between a binding domain and an adjacent constant domain or between a TCR chain and an adjacent self-cleaving peptide. Junction amino acids can result from the design of a construct that encodes a fusion protein (e.g., amino acid residues resulting from the use of a restriction enzyme site during the construction of a nucleic acid molecule encoding a fusion protein), or from cleavage of, for example, a self-cleaving peptide adjacent one or more domains of an encoded binding protein encompassed by the present invention (e.g., a P2A peptide disposed between a TCR α-chain and a TCR β-chain, the self-cleavage of which can leave one or more junction amino acids in the α-chain, the TCR β-chain, or both).

Engineered immune cells encompassed by the present invention may be administered as therapies for, e.g., a disorder characterized by MAGEA1 expression (such as a non-malignant disorder, a hyperproliferative disorder, or a relapse of a hyperproliferative disorder characterized by MAGEA1 expression). In some circumstances, it may be desirable to reduce or stop the activity associated with a cellular immunotherapy. Thus, in some embodiments, an engineered immune cell encompassed by the present invention comprises a heterologous polynucleotide encoding a binding protein and an accessory protein, such as a safety switch protein, which can be targeted using a cognate drug or other compound to selectively modulate the activity (e.g., lessen or ablate) of such cells when desirable. Safety switch proteins used in this regard include, for example, a truncated EGF receptor polypeptide (huEGFRt) that is devoid of extracellular N-terminal ligand binding domains and intracellular receptor tyrosine kinase activity but retains the native amino acid sequence, type I transmembrane cell surface localization, and a conformationally intact binding epitope for pharmaceutical-grade anti-EGFR monoclonal antibody, cetuximab (Erbitux) tEGF receptor (tEGFr; Wang et al. (2011) Blood 118:1255-1263), a caspase polypeptide (e.g., iCasp9; Straathof et al. (2005) Blood 105:4247-4254, Di Stasi et al. (2011) N. Engl. J. Med. 365:1673-1683, Zhou and Brenner (2016) Hematol. pii:S0301-472X: 30513-30516), RQR8 (Philip et al. (2014) Blood 124:1277-1287), and a human c-myc protein tag (Kieback et al. (2008) Proc. Natl. Acad. Sci. USA 105:623-628).

Other accessory components useful for therapeutic cells comprise a tag or selection marker (e.g., a CD34 enrichment tag) that allows the cells to be identified, sorted, isolated, enriched, or tracked. For example, marked immune cells having desired characteristics (e.g., an antigen-specific TCR and a safety switch protein) may be sorted away from unmarked cells in a sample and more efficiently activated and expanded for inclusion in a therapeutic product of desired purity.

As used herein, the term “selection marker” comprises a nucleic acid construct that confers an identifiable change to a cell permitting detection and positive selection of immune cells transduced with a polynucleotide comprising a selection marker. For example, RQR is a selection marker that comprises a major extracellular loop of CD20 and two minimal CD34 binding sites. In some embodiments, an RQR-encoding polynucleotide comprises a polynucleotide that encodes the 16 amino acid CD34 minimal epitope. In some embodiments, such as certain embodiments provided in the examples herein, the CD34 minimal epitope is incorporated at the amino terminal position of the CD8 stalk domain (Q8).

In further embodiments, the CD34 minimal binding site sequence may be combined with a target epitope for CD20 to form a compact marker/suicide gene for T cells (RQR8) (Philip et al. 2014). This construct allows for the selection of immune cells expressing the construct, with for example, CD34-specific antibody bound to magnetic beads (Miltenyi) and that utilizes clinically accepted pharmaceutical antibody, rituximab, that allows for the selective deletion of a transgene expressing engineered T cell (e.g., Philip et al. (2014) Blood 124:1277-1287, U.S. Pat. Publ. 2015-0093401, and U.S. Pat. Publ. 2018-0051089).

Further exemplary selection markers include several truncated type I transmembrane proteins normally not expressed on T cells: the truncated low-affinity nerve growth factor, truncated CD19, and truncated CD34 (e.g., Di Stasi et al. (2011) N. Engl. J. Med. 365:1673-1683, Mavilio et al. (1994) Blood 83:1988-1997, and Fehse et al. (2000) Mol. Ther. 7:448-456). A particularly attractive feature of CD19 and CD34 is the availability of the off-the-shelf Miltenyi CliniMACs™ selection system that can target these markers for clinical-grade sorting. However, CD19 and CD34 are relatively large surface proteins that may tax the vector packaging capacity and transcriptional efficiency of an integrating vector. Surface markers containing the extracellular, non-signaling domains or various proteins (e.g., CD19, CD34, LNGFR, etc.) also may be employed. Any selection marker may be employed and should be acceptable for good manufacturing practices. In some embodiments, selection markers are expressed with a polynucleotide that encodes a gene product of interest (e.g., a binding protein encompassed by the present invention, such as a TCR or CAR, or antigen-binding fragment thereof). Further examples of selection markers include, for example, reporters such as GFP, EGFP, β-gal or chloramphenicol acetyltransferase (CAT). In some embodiments, a selection marker, such as, for example, CD34 is expressed by a cell and the CD34 may be used to select enrich for, or isolate (e.g., by immunomagnetic selection) the transduced cells of interest for use in the methods described herein. As used herein, a CD34 marker is distinguished from an anti-CD34 antibody, or, for example, a scFv, TCR, or other antigen recognition moiety that binds to CD34.

In some embodiments, a selection marker comprises an RQR polypeptide, a truncated low-affinity nerve growth factor (tNGFR), a truncated CD19 (tCD19), a truncated CD34 (tCD34), or any combination thereof.

By way of background, inclusion of CD4⁺ T cells in an immunotherapy cell product can provide antigen-induced IL-2 secretion and augment persistence and function of transferred cytotoxic CD8⁺ T cells (e.g., Kennedy et al. (2008) Immunol. Rev. 222:129 and Nakanishi et al. Nature (2009) 52:510). In some embodiments, a class I-restricted TCR in CD4⁺ T cells may require the transfer of a CD8 co-receptor to enhance sensitivity of the TCR to class I HLA peptide complexes. CD4 co-receptors differ in structure to CD8 and cannot effectively substitute for CD8 co-receptors (e.g., Stone & Kranz (2013) Front. Immunol. 4:244 and Cole et al. (2012) Immunology 737:139). Thus, another accessory protein for use in the compositions and methods encompassed by the present invention comprises a CD8 co-receptor or component thereof. Engineered immune cells comprising a heterologous polynucleotide encoding a binding protein encompassed by the present invention may, in some embodiments, further comprise a heterologous polynucleotide encoding a CD8 co-receptor protein, or a beta-chain or alpha-chain component thereof.

A host cell may be efficiently transduced to contain, and may efficiently express, a single polynucleotide that encodes the binding protein, safety switch protein, selection marker, and CD8 co-receptor protein.

In one embodiment, the host cell encompassed by the present invention further includes a nucleic acid encoding a co-stimulatory molecule, such that the modified T cell expresses the co-stimulatory molecule. In some embodiments, the co-stimulatory domain is selected from CD3, CD27, CD28, CD83, CD86, CD127, 4-1BB, 4-1BBL, PD1 and PD1L.

In any of the foregoing embodiments, a host cell that express the binding protein described herein may be a universal immune cell. A “universal immune cell” comprises an immune cell that has been modified to reduce or eliminate expression of one or more endogenous genes that encode a polypeptide product selected from PD-1, LAG-3, CTLA4, TIM3, TIGIT, an HLA molecule, a TCR molecule, or any combination thereof. Without wishing to be bound by theory, certain endogenously expressed immune cell proteins may downregulate the immune activity of the modified immune cells (e.g., PD-1, LAG-3, CTLA4, TIGIT), or may interfere with the binding activity of a heterologously expressed binding protein encompassed by the present invention (e.g., an endogenous TCR that binds a non-MAGEA1 antigen and interferes with the modified immune cell binding to a target cell that expresses a MAGEA1 antigen such as a MAGEA1 immunogenic peptide in the context of an MHC molecule. Further, endogenous proteins (e.g., immune cell proteins, such as an HLA allele) expressed on a donor immune cell may be recognized as foreign by an allogeneic host, which may result in elimination or suppression of the modified donor immune cell by the allogeneic host.

Accordingly, decreasing or eliminating expression or activity of such endogenous genes or proteins can improve the activity, tolerance, or persistence of the modified immune cells in an autologous or allogeneic host setting, and allows universal administration of the cells (e.g., to any recipient regardless of HLA type). In some embodiments, cells in accordance with the present invention are syngeneic, meaning that they are genetically identical or sufficiently identical and immunologically compatible as to allow for transplantation. In some embodiments, a universal immune cell is a donor cell (e.g., allogeneic) or an autologous cell. In some embodiments, a modified immune cell (e.g., a universal immune cell) encompassed by the present invention comprises a chromosomal gene knockout of one or more of a gene that encodes PD-1, LAG-3, CTLA4, TIM3, TIGIT, or other immune checkpoint, an HLA component (e.g., a gene that encodes an α1 macroglobulin, an α2 macroglobulin, an α3 macroglobulin, a β1 microglobulin, or a β2 microglobulin), or a TCR component (e.g., a gene that encodes a TCR variable region or a TCR constant region) (see, e.g., Torikai el al. (2016) Nature Sci. Rep. 6:21757; Torikai et al. (2012) Blood 179:5697; and Torikai et al. (2013) Blood 722:1341, which also provide representative, exemplary gene editing techniques, compositions, and adoptive cell therapies useful according to the present invention).

As used herein, the term “chromosomal gene knockout” refers to a genetic alteration or introduced inhibitory agent in a host cell that prevents (e.g., reduces, delays, suppresses, or abrogates) production, by the host cell, of a functionally active endogenous polypeptide product. Alterations resulting in a chromosomal gene knockout may include, for example, introduced nonsense mutations (including the formation of premature stop codons), missense mutations, gene deletion, and strand breaks, as well as the heterologous expression of inhibitory nucleic acid molecules that inhibit endogenous gene expression in the host cell.

In some embodiments, a chromosomal gene knock-out or gene knock-in may be made by chromosomal editing of a host cell. Chromosomal editing may be performed using, for example, endonucleases. As used herein “endonuclease” refers to an enzyme capable of catalyzing cleavage of a phosphodiester bond within a polynucleotide chain. In some embodiments, an endonuclease is capable of cleaving a targeted gene thereby inactivating or “knocking out” the targeted gene. An endonuclease may be a naturally occurring, recombinant, genetically modified, or fusion endonuclease. The nucleic acid strand breaks caused by the endonuclease are commonly repaired through the distinct mechanisms of homologous recombination or non-homologous end joining (NHEJ). During homologous recombination, a donor nucleic acid molecule may be used for a donor gene “knock-in”, for target gene “knock-out”, and optionally to inactivate a target gene through a donor gene knock in or target gene knock out event. NHEJ is an error-prone repair process that often results in changes to the DNA sequence at the site of the cleavage, e.g., a substitution, deletion, or addition of at least one nucleotide. NHEJ may be used to “knock-out” a target gene. Examples of endonucleases include zinc finger nucleases, TALE-nucleases, CRISPR-Cas nucleases, meganucleases, and megaTALs.

As used herein, a “zinc finger nuclease” (ZFN) refers to a fusion protein comprising a zinc finger DNA-binding domain fused to a non-specific DNA cleavage domain, such as a Fokl endonuclease. Each zinc finger motif of about 30 amino acids binds to about 3 base pairs of DNA, and amino acids at certain residues may be changed to alter triplet sequence specificity (e.g., Desjarlais et al. (1993) Proc. Natl. Acad. Sci. 90:2256-2260 and Wolfe et al. (1999) J. Mol. Biol. 255:1917-1934). Multiple zinc finger motifs may be linked in tandem to create binding specificity to desired DNA sequences, such as regions having a length ranging from about 9 to about 18 base pairs. By way of background, ZFNs mediate genome editing by catalyzing the formation of a site-specific DNA double strand break (DSB) in the genome, and targeted integration of a transgene comprising flanking sequences homologous to the genome at the site of DSB is facilitated by homology directed repair. Alternatively, a DSB generated by a ZFN can result in knock out of target gene via repair by non-homologous end joining (NHEJ), which is an error-prone cellular repair pathway that results in the insertion or deletion of nucleotides at the cleavage site. In some embodiments, a gene knockout comprises an insertion, a deletion, a mutation or a combination thereof, made using a ZFN molecule.

As used herein, a “transcription activator-like effector nuclease” (TALEN) refers to a fusion protein comprising a TALE DNA-binding domain and a DNA cleavage domain, such as a Fokl endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the repeat variable diresidue (RVD), correlate with specific nucleotide recognition. The natural (canonical) code for DNA recognition of these TALEs has been determined such that an HD (histine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also well-known in the art (e.g., U.S. Pat. Publ. No. US 2011/0301073, which atypical RVDs are incorporated by reference herein in their entirety). TALENs may be used to direct site-specific double-strand breaks (DSB) in the genome of T cells. Non-homologous end joining (NHEJ) ligates DNA from both sides of a double-strand break in which there is little or no sequence overlap for annealing, thereby introducing errors that knock out gene expression. Alternatively, homology directed repair can introduce a transgene at the site of DSB providing homologous flanking sequences are present in the transgene. In some embodiments, a gene knockout comprises an insertion, a deletion, a mutation or a combination thereof, and made using a TALEN molecule.

As used herein, a “clustered regularly interspaced short palindromic repeats/Cas” (CRISPR/Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into three types (i.e., type I, type II, and type III) based on the sequence and structure of the Cas nucleases. The crRNA-guided surveillance complexes in types I and III need multiple Cas subunits. Type II system, the most studied, comprises at least three components: an RNA-guided Cas9 nuclease, a crRNA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA: tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a transgene with homologous flanking sequences may be introduced at the site of DSB via homology directed repair. The crRNA and tracrRNA may be engineered into a single guide RNA (sgRNA or gRNA) (e.g., Jinek et al. (2012) Science 337:816-821). Further, the region of the guide RNA complementary to the target site may be altered or programed to target a desired sequence (Xie et al. (2014) PLOS One 9:e100448, U.S. Pat. Publ. No. US 2014/0068797, U.S. Pat. Publ. No. US 2014/0186843, U.S. Pat. No. 8,697,359, and PCT Publ. No. WO 2015/071474). In some embodiments, a gene knockout comprises an insertion, a deletion, a mutation or a combination thereof, and made using a CRISPR/Cas nuclease system.

Exemplary gRNA sequences and methods of using the same to knock out endogenous genes that encode immune cell proteins include those described in Ren et al. (2017) Clin. Cancer Res. 23:2255-2266, which provides representative, exemplary gRNAs, CAS9 DNAS, vectors, and gene knockout techniques.

As used herein, a “meganuclease,” also referred to as a “homing endonuclease,” refers to an endodeoxyribonuclease characterized by a large recognition site (double stranded DNA sequences of about 12 to about 40 base pairs). Meganucleases may be divided into five families based on sequence and structure motifs: LAGLIDADG (SEQ ID NO: 38), GIY-YIG, HNH, His-Cys box, and PD-(D/E) XK. Exemplary meganucleases include I-Scel, I-Ceul, PI-PspI, RI-Sce, I-ScelV, I-Csml, I-Panl, I-Scell, I-Ppol, I-SceIII, I-Crel, I-Tevl, I-TevII and I-TevIII, whose recognition sequences are well-known (e.g., U.S. Pat. Nos. 5,420,032 and 6,833,252, Belfort et al. (1997) Nucl. Acids Res. 25:3379-3388, Dujon et al. (1989) Gene 52:115-118, Perler et al. (1994) Nucl. Acids Res. 22:1125-1127, Jasin (1996) Trends Genet. 72:224-228, Gimble et al. (1996) J. Mol. Biol. 263:163-180, and Argast et al. (1998) J. Mol. Biol. 280:345-353).

In some embodiments, naturally-occurring meganucleases may be used to promote site-specific genome modification of a target of interest, such as an immune checkpoint, an HLA-encoding gene, or a TCR component-encoding gene.

In other embodiments, an engineered meganuclease having a novel binding specificity for a target gene is used for site-specific genome modification (see, e.g., Porteus et al. (2005) Nat. Biotechnol. 23:967-73, Sussman et al. (2004) J. Mol. Biol. 342:31-41, Epinat et al. (2003) Nucl. Acids Res. 37:2952-2962, Chevalier et al. (2002) Mol. Cell 70:895-905, Ashworth et al. (2006) Nature 441:656-659, Paques et al. (2007) Curr. Gene Ther. 7:49-66, and U.S. Pat. Publ. Nos. US 2007/0117128, US 2006/0206949, US 2006/0153826, US 2006/0078552, and US 2004/0002092). In further embodiments, a chromosomal gene knockout is generated using a homing endonuclease that has been modified with modular DNA binding domains of TALENs to make a fusion protein known as a megaTAL. MegaTALs may be utilized to not only knock-out one or more target genes, but to also introduce (knock in) heterologous or exogenous polynucleotides when used in combination with an exogenous donor template encoding a polypeptide of interest.

In some embodiments, a chromosomal gene knockout comprises an inhibitory nucleic acid molecule that is introduced into a host cell (e.g., an immune cell) comprising a heterologous polynucleotide encoding an antigen-specific receptor that binds (e.g., specifically and/or selectively) to a MAGEA1 antigen, wherein the inhibitory nucleic acid molecule encodes a target-specific inhibitor and wherein the encoded target-specific inhibitor inhibits endogenous gene expression (i.e., of PD-1, TIM3, LAG3, CTLA4, TIGIT, an HLA component, or a TCR component, or any combination thereof) in the host immune cell.

A chromosomal gene knockout may be confirmed directly by DNA sequencing of the host immune cell following use of the knockout procedure or agent.

Chromosomal gene knockouts may also be inferred from the absence of gene expression (e.g., the absence of an mRNA or polypeptide product encoded by the gene) following the knockout.

In some embodiments, a host cell encompassed by the present invention is capable of specifically and/or selectively killing 50% or more of target cells that comprise a peptide-MHC (pMHC) complex comprising a MAGEA1 immunogenic peptide in the context of an MHC molecule.

In some embodiments, the modified immune cell is capable of producing a cytokine when contacted with target cells that comprise a peptide-MHC (pMHC) complex comprising a MAGEA1 immunogenic peptide in the context of an MHC molecule.

In some embodiments, the cytokine comprises IFN-γ or IL2. In some embodiments, the cytokine comprises TNF-α.

In some embodiments, the host cell is capable of producing a higher level of cytokine or a cytotoxic molecule when contacted with a target cell with expression of MAGEA1 at a level of less than or equal to about 1,000 transcript per million transcripts (TPM), 950 TPM, 900 TPM, 850 TPM, 800 TPM, 750 TPM, 700 TPM, 650 TPM, 600 TPM, 550 TPM, 500 TPM, 450 TPM, 400 TPM, 350 TPM, 300 TPM, 250 TPM, 200 TPM, 150 TPM, 100 TPM, 95 TPM, 90 TPM, 85 TPM, 80 TPM, 75 TPM, 70 TPM, 65 TPM, 60 TPM, 55 TPM, 50 TPM, 45 TPM, 40 TPM, 35 TPM, 34 TPM, 33 TPM, 32 TPM, 31 TPM, 30 TPM, 29 TPM, 28 TPM, 27 TPM, 26 TPM, 25 TPM, 24 TPM, 23 TPM, 22 TPM, 21 TPM, 20 TPM, 19 TPM, 18 TPM, 17 TPM, 16 TPM, 15 TPM, 14 TPM, 13 TPM, 12 TPM, 11 TPM, 10 TPM, 9 TPM, 8 TPM, 7 TPM, 6 TPM, 5 TPM, 4 TPM, 3 TPM, 2 TPM, and 1 TPM, or any range in between, inclusive, such as less than or equal to about 1,000 TPM to less than or equal to about 35 TPM). In some embodiments, the low MAGEA1 expression level is termed “heterozygous expression” meaning between about 1 TPM and about 35 TPM, or any range in between, inclusive, such as 1-32 TPM. For example, the host cell is capable of producing an at least 1.2 fold, 1.5 fold, 1.8 fold, 2.0 fold, 2.2 fold, 2.5 fold, 2.8 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 11 fold, 12 fold, 13 fold, 14 fold, 15 fold, 16 fold, 17 fold, 18 fold, 19 fold, 20 fold, 25 fold, 30 fold, 35 fold, 40 fold, 45 fold, 50 fold, 60 fold, 70 fold, 80 fold, 90 fold, 100 fold, 1000 fold, or more, or any range in between, inclusive, such as 1.2 fold to 2 fold, higher level of cytokine or a cytotoxic molecule.

In some embodiments, the host cell is capable of specifically and/or selectively killing a target cell expressing MAGEA1 (e.g., a hyperproliferative cell expressing MAGEA1). In certain embodiments, the target cell expresses a MAGEA1 immunogenic peptide in the context of an MHC molecule (e.g., a matched MHC molecule). In certain embodiments, the target cell expresses: (i) a polypeptide comprising or consisting of an amino acid sequence shown in Table 1; and (ii) a matched MHC molecule.

In some embodiments, host cells do not express MAGEA1 antigen, are not recognized by a binding protein described herein, are not of serotype HLA-A*02, and/or do not express an HLA-A*02 allele, such as HLA-A*02:01, HLA-A*02:02, HLA-A*02:03, HLA-A*02:05, HLA-A*02:06, or HLA-A*02:07 allele. For example, a patient may receive host cells from a healthy donor who is MAGEA1-negative or HLA-A*02:01-negative, or even autologous cells that have selected and/or engineered. Cells, such as stem cells like hematopoietic stem cells, isolated from that donor (or engineered autologous cells) may be used as the source of transplant material. In parallel, T cells isolated from the same donor may be genetically engineered to recognize MAGEA1, such as by expressing a MAGEA1 binding protein described herein. Donor cells, such as stem cells, may be used to engraft cell populations into the patient (e.g., hematopoietic stem cells used to reconstitute an immune system) and host cells may be infused into the patient with the goal of eliciting a highly specific anti-tumor effect. The engineered donor T cells may be designed to recognize and eliminate MAGEA1-expressing cells, such as all of the patient's native blood cells, including, for example, cancer cells like residual leukemia cells, which are MAGEA1-positive, thereby preventing relapse and promoting complete cures. Because patient's new healthy blood cells are derived from the donor and are therefore either MAGEA1-negative, HLA-A*02 serotype negative, and/or or HLA-A*02 allele-negative (e.g., negative for HLA-A*02:01 allele), engineered cells described herein may have minimal toxic side effects. Such patient-matched host cells and treatment methods may be used according to therapeutic methods described further below.

In some embodiments, the killing is determined by a killing assay. In some embodiment, the killing assay is carried out by co-culturing the host cell and the target cell at a ratio from 20:1 to 0.625:1, for example, from 15:1 to 1.25:1, from 10:1 to 1.5:1, from 8:1 to 3:1, from 6:1 to 5:1, 20:1 to 5:1, 10:1 to 2.5:1 etc. In some embodiments, the target cell is pulsed with 1 μg/mL to 50 μg/mL of MAGEA1 peptide, for example, from 1 μg/mL to 10 ng/ml, 500 ng/ml to 0.5 ng/ml, from 10 ng/ml to 10 μg/mL from 250 ng/ml to 1 ng/ml, from 50 ng/ml to 5 ng/mL, from 20 ng/ml to 10 ng/ml, etc.

In some embodiments, the host cell is capable of killing a higher number of target cells when contacted with target cells with a level of MAGEA1 less than or equal to about 1,000 transcript per million transcripts (TPM), 950 TPM, 900 TPM, 850 TPM, 800 TPM, 750 TPM, 700 TPM, 650 TPM, 600 TPM, 550 TPM, 500 TPM, 450 TPM, 400 TPM, 350 TPM, 300 TPM, 250 TPM, 200 TPM, 150 TPM, 100 TPM, 95 TPM, 90 TPM, 85 TPM, 80 TPM, 75 TPM, 70 TPM, 65 TPM, 60 TPM, 55 TPM, 50 TPM, 45 TPM, 40 TPM, 35 TPM, 34 TPM, 33 TPM, 32 TPM, 31 TPM, 30 TPM, 29 TPM, 28 TPM, 27 TPM, 26 TPM, 25 TPM, 24 TPM, 23 TPM, 22 TPM, 21 TPM, 20 TPM, 19 TPM, 18 TPM, 17 TPM, 16 TPM, 15 TPM, 14 TPM, 13 TPM, 12 TPM, 11 TPM, 10 TPM, 9 TPM, 8 TPM, 7 TPM, 6 TPM, 5 TPM, 4 TPM, 3 TPM, 2 TPM, and 1 TPM, or any range in between, inclusive, such as less than or equal to about 1,000 TPM to less than or equal to about 35 TPM). In some embodiments, the low MAGEA1 expression level is termed “heterozygous expression” meaning between about 1 TPM and about 35 TPM, or any range in between, inclusive, such as 1-32 TPM. For example, the host cell may be capable of killing an at least 1.2 fold, 1.5 fold, 1.8 fold, 2.0 fold, 2.2 fold, 2.5 fold, 2.8 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 11 fold, 12 fold, 13 fold, 14 fold, 15 fold, 16 fold, 17 fold, 18 fold, 19 fold, 20 fold, 25 fold, 30 fold, 35 fold, 40 fold, 45 fold, 50 fold, 60 fold, 70 fold, 80 fold, 90 fold, 100 fold, 1000 fold, or more, or any range in between, inclusive, such as 1.2 fold to 2 fold, higher number of target cells.

The present invention further provides a population of cells comprising at least one host cell described herein. The population of cells may be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cells, a muscle cell, a brain cell, etc. Alternatively, the population of cells may be a substantially homogeneous population, in which the population comprises mainly of host cells (e.g., consisting essentially of) comprising the recombinant expression vector. The population also may be a clonal population of cells, in which all cells of the population are clones of a single host cell comprising a recombinant expression vector, such that all cells of the population comprise the recombinant expression vector. In one embodiment encompassed by the present invention, the population of cells is a clonal population comprising host cells comprising a recombinant expression vector as described herein.

In an embodiment encompassed by the present invention, the numbers of cells in the population may be rapidly expanded. Expansion of the numbers of T cells may be accomplished by any of a number of methods as are well-known in the art (e.g., U.S. Pat. Nos. 8,034,334 and 8,383,099, U.S. Pat. Publ. No. 2012/0244133, Dudley et al. (2003) J. Immunother. 26:332-242, and Riddell et al. (1990) J. Immunol. Methods 128:189-201). For example, expansion of the numbers of T cells may be carried out by culturing the T cells with OKT3 antibody, IL-2, and feeder PBMC (e.g., irradiated allogeneic PBMC).

### VIII. Pharmaceutical Compositions

In another aspect encompassed by the present invention, pharmaceutical compositions are provided herein comprising compositions described herein (e.g., binding proteins, nucleic acids, cells, and the like) and a pharmaceutically acceptable carrier, diluent, or excipient.

The term “pharmaceutically acceptable” refers to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Agents and other compositions encompassed by the present invention may be specially formulated for administration in solid or liquid form, including those adapted for various routes of administration, such as (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound. Any appropriate form factor for an agent or composition described herein, such as, but not limited to, tablets, capsules, liquid syrups, soft gels, suppositories, and enemas, is contemplated.

Pharmaceutical compositions encompassed by the present invention may be presented as discrete dosage forms, such as capsules, sachets, or tablets, or liquids or aerosol sprays each containing a pre-determined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms may be prepared by any of the methods of pharmacy.

Suitable excipients include water, saline, dextrose, glycerol, or the like and combinations thereof. In some embodiments, compositions comprising host cells, binding proteins, or fusion proteins as disclosed herein further comprise a suitable infusion media. Suitable infusion media may be any isotonic medium formulation, typically normal saline, Normosol™-R (Abbott) or Plasma-Lyte™ A (Baxter), 5% dextrose in water, Ringer's lactate may be utilized. An infusion medium may be supplemented with human serum albumin or other human serum components. Unit doses comprising an effective amount of a host cell, or composition are also contemplated.

Also provided herein are unit doses that comprise an effective amount of a host cell or of a composition comprising the host cell. As described herein, host cells include immune cells, T cells (CD4⁺ T cells and/or CD8+ T cells), cytotoxic lymphocytes (e.g., cytotoxic T cells and/or natural killer (NK) cells), and the like. For example, in some embodiments, a unit dose comprises a composition comprising at least about 30%, at least about 40%, at least about 50%, at least about 60%), at least about 70%, at least about 80%, at least about 85%, at least about 90%, or at least about 95% engineered cells, either alone or in combination with other cells, such as comprising at least about 30%, at least about 40%, at least about 50%, at least about 60%), at least about 70%, at least about 80%, at least about 85%, at least about 90%, or at least about 95% other cells. In some embodiments, undesired cells are present at a reduced amount or substantially not present, such as less than about 50%, less than about 40%, less than about 30%, less than about 20%, less than about 10%, less than about 5%, or less then about 1% the population of cells in the composition.

The amount of cells in a composition or unit dose is at least one cell (for example, at least one engineered CD8⁺ T cell, engineered CD4⁺ T cell, and/or NK cell) or is more typically greater than 10² cells, for example, up to 10⁶, up to 10⁷, up to 10⁸ cells, up to 10⁹ cells, or more than 10¹⁰ cells. In some embodiments, the cells are administered in a range from about 106 to about 10¹⁰ cells/m², such as in a range of about 10⁵ to about 10⁹ cells/m². The number of cells will depend upon the ultimate use for which the composition is intended as well the type of cells included therein. For example, cells modified to contain a binding protein specific for a particular antigen will comprise a cell population containing at least 30%, 35%, 40%, 45%, 50%, 55%, 60%, 65%, 70%, 75%, 80%, 85%, 90%, 95% or more of such cells. For uses provided herein, cells are generally in a volume of a liter or less, 500 ml or less, 250 ml or less, or 100 ml or less. In embodiments, the density of the desired cells is typically greater than 10⁴ cells/ml and generally is greater than 10⁷ cells/ml, generally 10⁸ cells/ml or greater. The cells may be administered as a single infusion or in multiple infusions over a range of time. A clinically relevant number of immune cells may be apportioned into multiple infusions that cumulatively equal or exceed 10⁶, 10⁷, 10⁸, 10⁹, 10¹⁰, or 10¹¹ cells. In some embodiments, a unit dose of the engineered immune cells may be co-administered with (e.g., simultaneously or contemporaneously) hematopoietic stem cells from an allogeneic donor.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's condition, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity).

An effective amount of a pharmaceutical composition refers to an amount sufficient, at dosages and for periods of time needed, to achieve the desired clinical results or beneficial treatment, as described herein. An effective amount may be delivered in one or more administrations. If the administration is to a subject already known or confirmed to have a disease or disease-state, the term “therapeutically effective amount” may be used in reference to treatment, whereas “prophylactically effective amount” may be used to describe administrating an effective amount to a subject that is susceptible or at risk of developing a disease or disease-state (e.g., recurrence) as a preventative course.

The pharmaceutical compositions described herein may be presented in unit-dose or multi-dose containers, such as sealed ampoules or vials. Such containers may be frozen to preserve the stability of the formulation until infusion into the patient. In some embodiments, a unit dose comprises a host cell as described herein at a dose of about 10⁷ cells/m² to about 10¹¹ cells/m². The development of suitable dosing and treatment regimens for using the particular compositions described herein in a variety of treatment regimens, including e.g., parenteral or intravenous administration or formulation.

If the subject composition is administered parenterally, the composition may also include sterile aqueous or oleaginous solution or suspension. Suitable non-toxic parenterally acceptable diluents or solvents include water, Ringer's solution, isotonic salt solution, 1,3-butanediol, ethanol, propylene glycol or polythethylene glycols in mixtures with water. Aqueous solutions or suspensions may further comprise one or more buffering agents, such as sodium acetate, sodium citrate, sodium borate or sodium tartrate. Of course, any material used in preparing any dosage unit formulation should be pharmaceutically pure and substantially non-toxic in the amounts employed. In addition, the active compounds may be incorporated into sustained-release preparation and formulations. Dosage unit form, as used herein, refers to physically discrete units suited as unitary dosages for the subject to be treated; each unit may contain a predetermined quantity of engineered immune cells or active compound calculated to produce the desired effect in association with an appropriate pharmaceutical carrier.

In some embodiments, the pharmaceutical composition described herein and as described above for immunogenic compositions representatively exemplified for peptides, when administered to a subject, can elicit an immune response against a cell of interest that expresses MAGEA1. Such pharmaceutical compositions may be useful as vaccines for prophylactic and/or therapeutic treatment of a disorder characterized by MAGEA1 expression (e.g., a non-malignant disorder, a hyperproliferative disorder, or a relapse of a hyperproliferative disorder characterized by MAGEA1 expression).

In some embodiments, the pharmaceutical composition further comprises a physiologically acceptable adjuvant. In some embodiments, the adjuvant employed provides for increased immunogenicity of the pharmaceutical composition. Such a further immune response stimulating compound or adjuvant may be (i) admixed to the pharmaceutical composition in accordance with the present invention after reconstitution of the peptides and optional emulsification with an oil-based adjuvant as defined above, (ii) may be part of the reconstitution composition encompassed by the present invention defined above, (iii) may be physically linked to the peptide(s) to be reconstituted or (iv) may be administered separately to the subject, mammal or human, to be treated. The adjuvant may be one that provides for slow release of antigen (e.g., the adjuvant may be a liposome), or it may be an adjuvant that is immunogenic in its own right thereby functioning synergistically with antigens. For example, the adjuvant may be a known adjuvant or other substance that promotes antigen uptake, recruits immune system cells to the site of administration, or facilitates the immune activation of responding lymphoid cells. Adjuvants include, but are not limited to, immunomodulatory molecules (e.g., cytokines), oil and water emulsions, aluminum hydroxide, glucan, dextran sulfate, iron oxide, sodium alginate, bacto-adjuvant, synthetic polymers such as poly amino acids and co-polymers of amino acids, saponin, paraffin oil, and muramyl dipeptide. In some embodiments, the adjuvant is adjuvant 65, α-GalCer, aluminum phosphate, aluminum hydroxide, calcium phosphate, β-glucan peptide, CpG DNA, GM-CSF, GPI-0100, IFA, IFN-γ, IL-17, lipid A, lipopolysaccharide, Lipovant, Montanide™, N-acetyl-muramyl-L-alanyl-D-isoglutamine, pam3CSK4, quil A, trehalose dimycolate, or zymosan.

In some embodiments, the adjuvant is an immunomodulatory molecule. For example, the immunomodulatory molecule may be a recombinant protein cytokine, chemokine, or immunostimulatory agent or nucleic acid encoding cytokines, chemokines, or immunostimulatory agents designed to enhance the immunologic response.

Examples of immunomodulatory cytokines include interferons (e.g., IFNα, IFNβ and IFNγ), interleukins (e.g., IL-1, IL-2, IL-3, IL-4, IL-5, IL-6, IL-7, IL-8, IL-9, IL-10, IL-12, IL-17 and IL-20), tumor necrosis factors (e.g., TNFα and TNFβ), erythropoietin (EPO), FLT-3 ligand, gIp10, TCA-3, MCP-1, MIF, MIP-1.alpha., MIP-1B, Rantes, macrophage colony stimulating factor (M-CSF), granulocyte colony stimulating factor (G-CSF), and granulocyte-macrophage colony stimulating factor (GM-CSF), as well as functional fragments of any of the foregoing.

In some embodiments, an immunomodulatory chemokine that binds to a chemokine receptor, i.e., a CXC, CC, C, or CX3C chemokine receptor, also may be included in the compositions provided here. Examples of chemokines include, but are not limited to, Mip1α, Mip-1β, Mip-3α (Larc), Mip-3β, Rantes, Hcc-1, Mpif-1, Mpif-2, Mcp-1, Mcp-2, Mcp-3, Mcp-4, Mcp-5, Eotaxin, Tarc, Elc, 1309, IL-8, Gcp-2 Gro-α, Gro-β, Gro-γ, Nap-2, Ena-78, Gcp-2, Ip-10, Mig, I-Tac, Sdf-1, and Bca-1 (Blc), as well as functional fragments of any of the foregoing.

In some embodiments, the composition comprises a binding protein (e.g., a TCR, an antigen-binding fragment of a TCR, a CAR, or a fusion protein comprising a TCR and an effector domain), a TCRα and/or TCRβ polypeptide described herein. In some embodiments, the composition comprises a nucleic acid encoding a binding protein, a TCRα and/or TCRβ polypeptide described herein, such as a DNA molecule encoding a binding protein, a TCRα and/or TCRβ polypeptide. In some embodiments, the composition comprises an expression vector comprising an open reading frame encoding a binding protein, a TCRα and/or TCRβ polypeptide.

When taken up by a cell (e.g., T cells, NK cells, etc.), a DNA molecule may be present in the cell as an extrachromosomal molecule and/or may integrate into the chromosome. DNA may be introduced into cells in the form of a plasmid which may remain as separate genetic material. Alternatively, linear DNAs that may integrate into the chromosome may be introduced into the cell. Optionally, when introducing DNA into a cell, reagents which promote DNA integration into chromosomes may be added.

### IX. Uses and Methods

The compositions described herein may be used in a variety of diagnostic, prognostic, and therapeutic applications. In any method described herein, such as a diagnostic method, prognostic method, therapeutic method, or combination thereof, all steps of the method can be performed by a single actor or, alternatively, by more than one actor. For example, diagnosis can be performed directly by the actor providing therapeutic treatment. Alternatively, a person providing a therapeutic agent can request that a diagnostic assay be performed. The diagnostician and/or the therapeutic interventionist can interpret the diagnostic assay results to determine a therapeutic strategy. Similarly, such alternative processes can apply to other assays, such as prognostic assays.

In some uses and methods encompassed by the present invention, subjects or subject samples are utilized. In some embodiments, the subject is an animal. The animal may be of either sex and may be at any stage of development. In some embodiments, the animals is a vertebrate, such as a mammal. In some embodiments, the subject is a non-human mammal. In some embodiments, the subject is a domesticated animal, such as a dog, cat, cow, pig, horse, sheep, or goat. In some embodiments, the subject is a companion animal, such as a dog or cat. In some embodiments, the subject is a livestock animal, such as a cow, pig, horse, sheep, or goat. In some embodiments, the subject is a zoo animal. In some embodiments, the subject is a research animal, such as a rodent (e.g., mouse or rat), dog, pig, or non-human primate. In some embodiments, the animal is a genetically engineered animal. In some embodiments, the animal is a transgenic animal (e.g., transgenic mice and transgenic pigs). In some embodiments, the subject is a fish or reptile.

In some embodiments, the subject is a rodent, such as a mouse. In some such embodiments, the mouse is a transgenic mouse, such as a mouse expressing human MHC (i.e., HLA) molecules, such as HLA-B72 (e.g., Nicholson et al. (2012) Adv. Hematol. 2012:404081). In some embodiments, the subject is a transgenic mouse expressing human TCRs or is an antigen-negative mouse (e.g., Li et al. (2010) Nat. Med. 16:1029-1034 and Obenaus et al. (2015) Nat. Biotechnol. 33:402-407). In some embodiments, the subject is a transgenic mouse expressing human HLA molecules and human TCRs. In some embodiments, such as where the subject is a transgenic HLA mouse, the identified TCRs are modified, e.g., to be chimeric or humanized. In some embodiments, the TCR scaffold is modified, such as analogous to known binding protein humanizing methods.

In some embodiments, the subject is a human. In some embodiments, the subject is an animal model of a disorder characterized by MAGEA1 expression (e.g., a non-malignant disorder, the hyperproliferative disorder, or the relapse of a hyperproliferative disorder characterized by expression of a MAGEA1 antigen). For example, the animal model may be an orthotopic xenograft animal model of a human-derived cancer.

In some embodiments, the subject is a human, such as a human with a disorder characterized by MAGEA1 expression.

The methods described herein may be used to treat a subject in need thereof. As used herein, a “subject in need thereof” includes any subject who has a disorder characterized by MAGEA1 expression, a relapse of a disorder characterized by MAGEA1 expression, and/or who is predisposed to a disorder characterized by MAGEA1 expression. As described herein, a disorder characterized by MAGEA1 expression may be a non-malignant disorder, a hyperproliferative disorder, or a relapse of a hyperproliferative disorder characterized by MAGEA1 expression.

In some embodiments of the methods encompassed by the present invention, the subject has not undergone treatment for a disorder characterized by MAGEA1 expression, such as chemotherapy, radiation therapy, targeted therapy, and/or immunotherapies. In some embodiments, the subject has undergone treatment for a disorder characterized by MAGEA1 expression, such as chemotherapy, radiation therapy, targeted therapy, and/or immunotherapies.

In some embodiments, the subject has had surgery to remove cancerous or precancerous tissue. In some embodiments, the cancerous tissue has not been removed, e.g., the cancerous tissue may be located in an inoperable region of the body, such as in a tissue that is essential for life, or in a region where a surgical procedure would cause considerable risk of harm to the patient.

In some embodiments, the subject or cells thereof are resistant to a therapy of relevance, such as resistant to standard of care therapy, immune checkpoint inhibitor therapy, and the like. For example, modulating one or more biomarkers encompassed by the present invention may overcome resistance to immune checkpoint inhibitor therapy.

In some embodiments, the subjects are in need of modulation according to compositions and methods described herein, such as having been identified as having an unwanted absence, presence, or aberrant MAGEA1 expression.

a. Diagnostic Methods

In an aspect encompassed by the present invention, provided herein are diagnostic methods for detecting the presence or absence of a MAGEA1 antigen, a MAGEA1 antigen-MHC complex, a cell of interest expressing MAGEA1, and/or a cell having had exposure to MAGEA1, comprising detecting the presence or absence of said MAGEA1 antigen in a sample by use of at least one binding protein, or at least one host cell described herein. In some embodiments, the method further comprising obtaining the sample (e.g., from a subject). In some embodiments, the at least one binding protein or the at least one host cell, forms a complex with a MAGEA1 peptide epitope in the context of an MHC molecule, and the complex is detected in the form of fluorescence activated cell sorting (FACS), enzyme linked immunosorbent assay (ELISA), radioimmune assay (RIA), immunochemically, Western blot, or intracellular flow assay.

In an aspect encompassed by the present invention, provided herein are diagnostic methods for detecting the level of MAGEA1 or a disorder characterized by MAGEA1 expression, such as a non-malignant disorder, a hyperproliferative disorder, or a relapse of a hyperproliferative disorder characterized by MAGEA1 expression in a subject, comprising: a) contacting a sample obtained from the subject with at least one agent (e.g., a MAGEA1 immunogenic peptide, MAGEA1 immunogenic peptide-MHC complex (pMHC), binding protein, at least one host cell, or a population of host cells described herein); and b) detecting the level of reactivity, wherein a higher level of reactivity compared to a control level indicates the level of a disorder characterized by MAGEA1 expression (e.g., a non-malignant disorder, a hyperproliferative disorder, or a relapse of a hyperproliferative disorder characterized by MAGEA1 expression) in the subject.

In some embodiments, the level of reactivity is indicated by T cell activation or effector function, such as, but not limited to, T cell proliferation, killing, or cytokine release. The control level may be a reference number or a level of a healthy subject who has no exposure to a disorder characterized by MAGEA1 expression (e.g., a non-malignant disorder, a hyperproliferative disorder, or a relapse of a hyperproliferative disorder characterized by MAGEA1 expression).

A biological sample may be obtained from a subject for determining the presence and level of an immune response to the papteid antigen (e.g., a MAGEA1 antigen) as described herein. A “biological sample” as used herein may be a blood sample (from which serum or plasma may be prepared), biopsy specimen, body fluids (e.g., blood, isolated PBMCs, isolated T cells, lung lavage, ascites, mucosal washings, synovial fluid, etc.), bone marrow, lymph nodes, tissue explant, organ culture, or any other tissue or cell preparation from the subject or a biological source. Biological samples may also be obtained from the subject prior to receiving any pharmaceutical composition, which biological sample is useful as a control for establishing baseline data.

Antigen-specific T cell responses are typically determined by comparisons of observed T cell responses according to any of the herein described T cell functional parameters (e.g., proliferation, cytokine release, CTL activity, altered cell surface marker phenotype, etc.) that may be made between T cells that are exposed to a cognate antigen in an appropriate context (e.g., the antigen used to prime or activate the T cells, when presented by immunocompatible antigen-presenting cells) and T cells from the same source population that are exposed instead to a structurally distinct or irrelevant control antigen. A response to the cognate antigen that is greater, with statistical significance, than the response to the control antigen signifies antigen-specificity.

The level of an immune response, such as a cytotoxic T lymphocyte (CTL) immune response, may be determined by any one of numerous immunological methods described herein and routinely practiced in the art. For example, the level of a CTL immune response may be determined prior to and following administration of any one of the herein described binding proteins expressed by, for example, a T cell. Cytotoxicity assays for determining CTL activity may be performed using any one of several techniques and methods routinely practiced in the art (e.g., Henkart el al., “Cytotoxic T-Lymphocytes” in Fundamental Immunology, Paul (ed.) (2003 Lippincott Williams & Wilkins, Philadelphia, PA), pages 1127-50, and references cited therein).

The present invention provides, in part, methods, systems, and code for accurately classifying whether a biological sample is associated with an output of interest, such as expression of a target of interest, such as MAGEA1. In some embodiments, the present invention is useful for classifying a sample (e.g., from a subject) as associated with or at risk for responding to or not responding to therapy for a disorder characterized by MAGEA1 expression using a statistical algorithm and/or empirical data.

An exemplary method for detecting the amount or activity of MAGEA1, and thus useful for classifying whether a sample is likely or unlikely to respond to a therapy for a disorder characterized by MAGEA1 expression involves contacting a biological sample with an agent, such as a MAGEA1 immunogenic peptide or binding agent described herein, capable of detecting the amount or activity of MAGEA1 in the biological sample. In some embodiments, the method further comprise obtaining a biological sample, such as from a test subject. In some embodiments, at least one agent is used, wherein two, three, four, five, six, seven, eight, nine, ten, or more such agents may be used in combination (e.g., in sandwich ELISAs) or in serial. In certain instances, the statistical algorithm is a single learning statistical classifier system. For example, a single learning statistical classifier system may be used to classify a sample as a based upon a prediction or probability value and the presence or level of the biomarker. The use of a single learning statistical classifier system typically classifies the sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well-known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method encompassed by the present invention further comprises sending the sample classification results to a clinician, e.g., an oncologist.

In some embodiments, the diagnosis of a subject (e.g., including HLA typing and/or loss of heterozygosity (LOH) to determine compatibility with TCR-HLA complex binding by TCRs of interest) is followed by administering to the individual a therapeutically effective amount of a defined treatment based upon the diagnosis.

In some embodiments, the methods further involve obtaining a control biological sample (e.g., biological sample from a subject who does not have a disorder characterized by MAGEA1 expression, a subject who is in remission, a subject whose disorder is susceptible to therapy, a subject whose disorder is progressing, or other subjects of interest).

In some embodiments of analytical methods described herein, MAGEA1 expression (e.g., in a sample from a subject) is compared to a pre-determined control (standard) sample. The sample from the subject is typically from a diseased tissue, such as cancer cells or tissues. The control sample may be from the same subject or from a different subject. The control sample is typically a normal, non-diseased sample. However, in some embodiments, such as for staging of disease or for evaluating the efficacy of treatment, the control sample may be from a diseased tissue. The control sample may be a combination of samples from several different subjects. In some embodiments, the MAGEA1 expression measurement(s) from a subject is compared to a pre-determined level. This pre-determined level is typically obtained from normal samples. As described herein, a “pre-determined” expression may be used to, by way of example only, evaluate a subject that may be selected for treatment, evaluate a response to cancer, and/or evaluate a response to a combination cancer therapy. A pre-determined biomarker amount and/or activity measurement(s) may be determined in populations of patients with or without a disorder characterized by MAGEA1 expression. The pre-determined biomarker amount and/or activity measurement(s) may be a single number, equally applicable to every patient, or the pre-determined biomarker amount and/or activity measurement(s) may vary according to specific sub-populations of patients. Age, weight, height, and other factors of a subject may affect the pre-determined biomarker amount and/or activity measurement(s) of the individual. Furthermore, the pre-determined biomarker amount and/or activity may be determined for each subject individually. In one embodiment, the amounts determined and/or compared in a method described herein are based on absolute measurements.

In another embodiment, the amounts determined and/or compared in a method described herein are based on relative measurements, such as ratios (e.g., biomarker copy numbers, level, and/or activity before a treatment vs. after a treatment, such biomarker measurements relative to a spiked or man-made control, such biomarker measurements relative to the expression of a housekeeping gene, and the like). For example, the relative analysis may be based on the ratio of pre-treatment biomarker measurement as compared to post-treatment biomarker measurement. Pre-treatment biomarker measurement may be made at any time prior to initiation of a therapy. Post-treatment biomarker measurement may be made at any time after initiation of therapy. In some embodiments, post-treatment biomarker measurements are made 1, 2, 3, 4, 5, 6, 7, 8, 9, 10, 11, 12, 13, 14, 15, 16, 17, 18, 19, 20 weeks or more after initiation of therapy, and even longer toward indefinitely for continued monitoring. Treatment may comprise therapy to treat the disorder characterized by MAGEA1 expression, either alone or in combination with other agents, such as anti-cancer agents like chemotherapy or immune checkpoint inhibitors.

The pre-determined MAGEA1 expression may be any suitable standard. For example, the pre-determined MAGEA1 expression may be obtained from the same or a different subject for whom a subject selection is being assessed. In one embodiment, the pre-determined biomarker amount and/or activity measurement(s) may be obtained from a previous assessment of the same patient. In such a manner, the progress of the selection of the patient may be monitored over time. In addition, the control may be obtained from an assessment of another human or multiple humans, e.g., selected groups of humans, if the subject is a human. In such a manner, the extent of the selection of the human for whom selection is being assessed may be compared to suitable other humans, e.g., other humans who are in a similar situation to the human of interest, such as those suffering from similar or the same condition(s) and/or of the same ethnic group.

In some embodiments encompassed by the present invention the change of MAGEA1 expression from the pre-determined level is about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 fold or greater, or any range in between, inclusive. Such cut-off values apply equally when the measurement is based on relative changes, such as based on the ratio of pre-treatment biomarker measurement as compared to post-treatment biomarker measurement.

In some embodiments, MAGEA1 expression may be detected and/or quantified by detecting or quantifying MAGEA1 polypeptide or antigen thereof, such as by using a composition described herein. The polypeptide may be detected and quantified by any of a number of means well-known to those of skill in the art, such as by immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991).

b. Therapeutic Methods

In an aspect encompassed by the present invention, provided herein are methods for preventing and/or treating a disorder characterized by MAGEA1 expression (e.g., a non-malignant disorder, a hyperproliferative disorder, or a relapse of a hyperproliferative disorder characterized by MAGEA1 expression) and/or for inducing an immune response against a cell of interest, such as a hyperproliferative cell, expressing MAGEA1. In some embodiments, the method comprises administering to a subject a therapeutically effective amount of a composition described herein, such as an immunogenic composition, such as a composigion comprising cells expressing at least one binding protein, and the like. The methods encompassed by the present invention also may be used to determine the responsiveness to cancer therapy of many different disorders characterized by MAGEA1 expression in subjects such as those described herein.

In some embodiments, the disorder characterized by MAGEA1 expression is a cancer. The terms “cancer” or “tumor” or “hyperproliferative” refer to the presence of cells possessing characteristics typical of cancer-causing cells, such as uncontrolled proliferation, immortality, invasive or metastatic potential, rapid growth, and certain characteristic morphological features. In some embodiments, such cells exhibit such characteristics in part or in full due to the expression and activity of immune checkpoint proteins, such as PD-1, PD-L1, PD-L2, and/or CTLA-4.

Cancer cells are often in the form of a tumor, but such cells may exist alone within an animal, or may be a non-tumorigenic cancer cell, such as in a hematologic cancer like leukemia. As used herein, the term “cancer” includes premalignant as well as malignant cancers. Cancers include, but are not limited to, a variety of cancers, carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma); hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma; hematopoietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma, and schwannomas; tumors of mesenchymal origin including fibrosarcoma, rhabdomyosarcoma, and osteosarcoma; other tumors including melanoma, xenoderma pigmentosum, keratoactanthoma, seminoma, thyroid follicular cancer, and teratocarcinoma; melanoma, unresectable stage III or IV malignant melanoma, squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, head and neck cancer, gastric cancer, germ cell tumor, bone cancer, bone tumors, adult malignant fibrous histiocytoma of bone; childhood, malignant fibrous histiocytoma of bone, sarcoma, pediatric sarcoma, sinonasal natural killer, neoplasms, plasma cell neoplasm; myelodysplastic syndromes; neuroblastoma; testicular germ cell tumor, intraocular melanoma, myelodysplastic syndromes; myelodysplastic/myeloproliferative diseases, synovial sarcoma, chronic myeloid leukemia, acute lymphoblastic leukemia, Philadelphia chromosome positive acute lymphoblastic leukemia (Ph+ ALL), multiple myeloma, acute myelogenous leukemia, chronic lymphocytic leukemia, mastocytosis and any symptom associated with mastocytosis, and any metastasis thereof. In addition, disorders include urticaria pigmentosa, mastocytosis such as diffuse cutaneous mastocytosis, solitary mastocytoma in human, as well as dog mastocytoma and some rare subtypes like bullous, erythrodermic and telangiectatic mastocytosis, mastocytosis with an associated hematological disorder, such as a myeloproliferative or myelodysplastic syndrome, or acute leukemia, myeloproliferative disorder associated with mastocytosis, mast cell leukemia, in addition to other cancers. Other cancers are also included within the scope of disorders including, but are not limited to, the following: carcinoma, including that of the bladder, urothelial carcinoma, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma, and any metastasis thereof. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, bone cancer, brain tumor, lung carcinoma (including lung adenocarcinoma), small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In some embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated. In some embodiments, the cancer is selected from the group consisting of (advanced) non-small cell lung cancer, melanoma, head and neck squamous cell cancer, (advanced) urothelial bladder cancer, (advanced) kidney cancer (RCC), microsatellite instability-high cancer, classical Hodgkin lymphoma, (advanced) gastric cancer, (advanced) cervical cancer, primary mediastinal B-cell lymphoma, (advanced) hepatocellular carcinoma, colorectal cancer, gastrointestinal cancer, breast invasive carcinoma, bladder urothelial carcinoma, and (advanced) merkel cell carcinoma.

In addition, the compositions described herein may also be administered in combination therapy to further modulate a desired activity. Additional agents include, without limitations, chemotherapeutic agents, hormones, antiangiogens, radiolabelled, compounds, or with surgery, cryotherapy, and/or radiotherapy. The preceding treatment methods may be administered in conjunction with other forms of conventional therapy (e.g., standard-of-care treatments for cancer well-known to the skilled artisan), either consecutively with, pre- or post-conventional therapy. For example, these modulatory agents may be administered with a therapeutically effective dose of chemotherapeutic agent. In another embodiment, these modulatory agents are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular melanoma, being treated, the extent of the disease and other factors familiar to the physician of skill in the art and may be determined by the physician.

Therapy using one or more compositions described herein, either alone or in combination with other therapies, such as cancer therapies, may be used to contact MAGEA1-expressing cells and/or administered to a desired subject, such as a subject that is indicated as being a likely responder to therapy. In another embodiment, such therapy may be avoided once a subject is indicated as not being a likely responder to the therapy (e.g., as assessed according to a diagnostic or prognostic method described herein) and an alternative treatment regimen, such as targeted and/or untargeted cancer therapies, may be recommended and/or administered.

The term “targeted therapy” refers to administration of agents that selectively interact with a chosen biomolecule to thereby treat cancer. For example, targeted therapy regarding the inhibition of immune checkpoint inhibitor is useful in combination with the methods encompassed by the present invention.

The term “immunotherapy” or “immunotherapies” generally refers to any strategy for modulating an immune response in a beneficial manner and encompasses the treatment of a subject afflicted with, or at risk of contracting or suffering a recurrence of, a disease by a method comprising inducing, enhancing, suppressing or otherwise modifying an immune response, as well as any treatment that uses certain parts of a subject's immune system to fight diseases, such as cancer. The subject's own immune system is stimulated (or suppressed), with or without administration of one or more agent for that purpose. Immunotherapies that are designed to elicit or amplify an immune response are referred to as “activation immunotherapies.” Immunotherapies that are designed to reduce or suppress an immune response are referred to as “suppression immunotherapies.” In some embodiments, an immunotherapy is specific for cells of interest, such as cancer cells. In some embodiments, immunotherapy may be “untargeted,” which refers to administration of agents that do not selectively interact with immune system cells, yet modulates immune system function. Representative examples of untargeted therapies include, without limitation, chemotherapy, gene therapy, and radiation therapy.

Some forms of immunotherapy are targeted therapies that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy may involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy may also focus on using the cytotoxic lymphocyte-recognized epitopes of cancer cell lines. Alternatively, antisense polynucleotides, ribozymes, RNA interference molecules, triple helix polynucleotides and the like, may be used to selectively modulate biomolecules that are linked to the initiation, progression, and/or pathology of a tumor or cancer. Similarly, immunotherapy may take the form of cell-based therapies. For example, adoptive cellular immunotherapy is a type of immunotherapy using immune cells, such as T cells, that have a natural or genetically engineered reactivity to a patient's cancer are generated and then transferred back into the cancer patient. The injection of a large number of activated tumor-specific T cells may induce complete and durable regression of cancers.

Immunotherapy may involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy may also focus on using the cytotoxic lymphocyte-recognized epitopes of cancer cell lines. Alternatively, antisense polynucleotides, ribozymes, RNA interference molecules, triple helix polynucleotides and the like, may be used to selectively modulate biomolecules that are linked to the initiation, progression, and/or pathology of a tumor or cancer.

In some embodiments, an immunotherapeutic agent is an agonist of an immune-stimulatory molecule; an antagonist of an immune-inhibitory molecule; an antagonist of a chemokine; an agonist of a cytokine that stimulates T cell activation; an agent that antagonizes or inhibits a cytokine that inhibits T cell activation; and/or an agent that binds to a membrane bound protein of the B7 family. In some embodiments, the immunotherapeutic agent is an antagonist of an immune-inhibitory molecule. In some embodiments, the immunotherapeutic agents may be agents for cytokines, chemokines and growth factors, for examples, neutralizing antibodies that neutralize the inhibitory effect of tumor associated cytokines, chemokines, growth factors and other soluble factors, including IL-10, TGF-β and VEGF.

In some embodiments, immunotherapy comprises inhibitors of one or more immune checkpoints. The term “immune checkpoint” refers to a group of molecules on the cell surface of CD4+ and/or CD8+ T cells that fine-tune immune responses by modulating anti-cancer immune responses, such as down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, ICOS, HVEM, PD-L2, CD200R, CD160, gp49B, PIR-B, KRLG-1, KIR family receptors, TIM-1, TIM-3, TIM-4, LAG-3 (CD223), IDO, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR (see, for example, WO 2012/177624). The term further encompasses biologically active protein fragments, as well as nucleic acids encoding full-length immune checkpoint proteins.

Some immune checkpoints are “immune-inhibitory immune checkpoints” encompassing molecules (e.g., proteins) that inhibit, down-regulate, or suppress a function of the immune system (e.g., an immune response). For example, PD-L1 (programmed death-ligand 1), also known as CD274 or B7-H1, is a protein that transmits an inhibitory signal that reduces proliferation of T cells to suppress the immune system. CTLA-4 (cytotoxic T-lymphocyte-associated protein 4), also known as CD152, is a protein receptor on the surface of antigen-presenting cells that serves as an immune checkpoint (“off” switch) to downregulate immune responses. TIM-3 (T-cell immunoglobulin and mucin-domain containing-3), also known as HAVCR2, is a cell surface protein that serves as an immune checkpoint to regulate macrophage activation. VISTA (V-domain Ig suppressor of T cell activation) is a type I transmembrane protein that functions as an immune checkpoint to inhibit T cell effector function and maintain peripheral tolerance. LAG-3 (lymphocyte-activation gene 3) is an immune checkpoint receptor that negatively regulates proliferation, activation, and homeostasis of T cells. BTLA (B- and T-lymphocyte attenuator) is a protein that displays T cell inhibition via interactions with tumor necrosis family receptors (TNF-R). KIR (killer-cell immunoglobulin-like receptor) is a family of proteins expressed on NK cells, and a minority of T cells, that suppress the cytotoxic activity of NK cells. In some embodiments, immunotherapeutic agents may be agents specific to immunosuppressive enzymes such as inhibitors that may block the activities of arginase (ARG) and indoleamine 2,3-dioxygenase (IDO), an immune checkpoint protein that suppresses T cells and NK cells, which change the catabolism of the amino acids arginine and tryptophan in the immunosuppressive tumor microenvironment. The inhibitors may include, but are not limited to, N-hydroxy-L-Arg (NOHA) targeting to ARG-expressing M2 macrophages, nitroaspirin or sildenafil (Viagra®), which blocks ARG and nitric oxide synthase (NOS) simultaneously; and IDO inhibitors, such as 1-methyl-tryptophan. The term further encompasses biologically active protein fragment, as well as nucleic acids encoding full-length immune checkpoint proteins and biologically active protein fragments thereof. In some embodiment, the term further encompasses any fragment according to homology descriptions provided herein.

By contrast, other immune checkpoints are “immune-stimulatory” encompassing molecules (e.g., proteins) that activate, stimulate, or promote a function of the immune system (e.g., an immune response). In some embodiments, the immune-stimulatory molecule is CD28, CD80 (B7.1), CD86 (B7.2), 4-1BB (CD137), 4-1BBL (CD137L), CD27, CD70, CD40, CD40L, CD122, CD226, CD30, CD30L, OX40, OX40L, HVEM, BTLA, GITR and its ligand GITRL, LIGHT, LTBR, LTaβ, ICOS (CD278), ICOSL (B7-H2), and NKG2D. CD40 (cluster of differentiation 40) is a costimulatory protein found on antigen presenting cells that is required for their activation. OX40, also known as tumor necrosis factor receptor superfamily member 4 (TNFRSF4) or CD134, is involved in maintenance of an immune response after activation by preventing T-cell death and subsequently increasing cytokine production. CD137 is a member of the tumor necrosis factor receptor (TNF-R) family that co-stimulates activated T cells to enhance proliferation and T cell survival. CD122 is a subunit of the interleukin-2 receptor (IL-2) protein, which promotes differentiation of immature T cells into regulatory, effector, or memory T cells. CD27 is a member of the tumor necrosis factor receptor superfamily and serves as a co-stimulatory immune checkpoint molecule. CD28 (cluster of differentiation 28) is a protein expressed on T cells that provides co-stimulatory signals required for T cell activation and survival. GITR (glucocorticoid-induced TNFR-related protein), also known as TNFRSF18 and AITR, is a protein that plays a key role in dominant immunological self-tolerance maintained by regulatory T cells. ICOS (inducible T-cell co-stimulator), also known as CD278, is a CD28-superfamily costimulatory molecule that is expressed on activated T cells and play a role in T cell signaling and immune responses.

Immune checkpoints and their sequences are well-known in the art and representative embodiments are described further below. Immune checkpoints generally relate to pairs of inhibitory receptors and the natural binding partners (e.g., ligands). For example, PD-1 polypeptides are inhibitory receptors capable of transmitting an inhibitory signal to an immune cell to thereby inhibit immune cell effector function, or are capable of promoting costimulation (e.g., by competitive inhibition) of immune cells, e.g., when present in soluble, monomeric form. Preferred PD-1 family members share sequence identity with PD-1 and bind to one or more B7 family members, e.g., B7-1, B7-2, PD-1 ligand, and/or other polypeptides on antigen presenting cells. The term “PD-1 activity,” includes the ability of a PD-1 polypeptide to modulate an inhibitory signal in an activated immune cell, e.g., by engaging a natural PD-1 ligand on an antigen presenting cell. Modulation of an inhibitory signal in an immune cell results in modulation of proliferation of, and/or cytokine secretion by, an immune cell. Thus, the term “PD-1 activity” includes the ability of a PD-1 polypeptide to bind its natural ligand(s), the ability to modulate immune cell inhibitory signals, and the ability to modulate the immune response. The term “PD-1 ligand” refers to binding partners of the PD-1 receptor and includes both PD-L1 (Freeman et al. (2000) J. Exp. Med. 192:1027-1034) and PD-L2 (Latchman et al. (2001) Nat. Immunol. 2:261). The term “PD-1 ligand activity” includes the ability of a PD-1 ligand polypeptide to bind its natural receptor(s) (e.g., PD-1 or B7-1), the ability to modulate immune cell inhibitory signals, and the ability to modulate the immune response.

As used herein, the term “immune checkpoint therapy” refers to the use of agents that inhibit immune-inhibitory immune checkpoints, such as inhibiting their nucleic acids and/or proteins. Inhibition of one or more such immune checkpoints may block or otherwise neutralize inhibitory signaling to thereby upregulate an immune response in order to more efficaciously treat cancer. Exemplary agents useful for inhibiting immune checkpoints include antibodies, small molecules, peptides, peptidomimetics, natural ligands, and derivatives of natural ligands, that may either bind and/or inactivate or inhibit immune checkpoint proteins, or fragments thereof; as well as RNA interference, antisense, nucleic acid aptamers, etc. that may downregulate the expression and/or activity of immune checkpoint nucleic acids, or fragments thereof. Exemplary agents for upregulating an immune response include antibodies against one or more immune checkpoint proteins that block the interaction between the proteins and its natural receptor(s); a non-activating form of one or more immune checkpoint proteins (e.g., a dominant negative polypeptide); small molecules or peptides that block the interaction between one or more immune checkpoint proteins and its natural receptor(s); fusion proteins (e.g., the extracellular portion of an immune checkpoint inhibition protein fused to the Fc portion of an antibody or immunoglobulin) that bind to its natural receptor(s); nucleic acid molecules that block immune checkpoint nucleic acid transcription or translation; and the like. Such agents may directly block the interaction between the one or more immune checkpoints and its natural receptor(s) (e.g., antibodies) to prevent inhibitory signaling and upregulate an immune response. Alternatively, agents may indirectly block the interaction between one or more immune checkpoint proteins and its natural receptor(s) to prevent inhibitory signaling and upregulate an immune response. For example, a soluble version of an immune checkpoint protein ligand such as a stabilized extracellular domain may binding to its receptor to indirectly reduce the effective concentration of the receptor to bind to an appropriate ligand. In one embodiment, anti-PD-1 antibodies, anti-PD-L1 antibodies, and/or anti-PD-L2 antibodies, either alone or in combination, are used to inhibit immune checkpoints. Therapeutic agents used for blocking the PD-1 pathway include antagonistic antibodies and soluble PD-L1 ligands. The antagonist agents against PD-1 and PD-L1/2 inhibitory pathway may include, but are not limited to, antagonistic antibodies to PD-1 or PD-L1/2 (e.g., 17D8, 2D3, 4H1, 5C4 (also known as nivolumab or BMS-936558), 4A11, 7D3 and 5F4 disclosed in U.S. Pat. No. 8,008,449; AMP-224, pidilizumab (CT-011), pembrolizumab, and antibodies disclosed in U.S. Pat. Nos. 8,779,105; 8,552,154; 8,217,149; 8,168,757; 8,008,449; 7,488,802; 7,943,743; 7,635,757; and 6,808,710. Similarly, additional representative checkpoint inhibitors may be, but are not limited to, antibodies against inhibitory regulator CTLA-4 (anti-cytotoxic T-lymphocyte antigen 4 anti-cytotoxic T-lymphocyte antigen 4), such as ipilimumab, tremelimumab (fully humanized), anti-CD28 antibodies, anti-CTLA-4 adnectins, anti-CTLA-4 domain antibodies, single chain anti-CTLA-4 antibody fragments, heavy chain anti-CTLA-4 fragments, light chain anti-CTLA-4 fragments, and other antibodies, such as those disclosed in U.S. Pat. Nos. 8,748,815; 8,529,902; 8,318,916; 8,017,114; 7,744,875; 7,605,238; 7,465,446; 7,109,003; 7,132,281; 6,984,720; 6,682,736; 6,207,156; and 5,977,318, as well as EP Pat. No. 1212422, U.S. Pat Publ. Numbers 2002/0039581 and 2002/086014, and Hurwitz et al. (1998) Proc. Natl. Acad. Sci. U.S.A. 95:10067-10071.

The representative definitions of immune checkpoint activity, ligand, blockade, and the like exemplified for PD-1, PD-L1, PD-L2, and CTLA-4 apply generally to other immune checkpoints.

The term “untargeted therapy” refers to administration of agents that do not selectively interact with a chosen biomolecule yet treat cancer. Representative examples of untargeted therapies include, without limitation, chemotherapy, gene therapy, and radiation therapy.

In one embodiment, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolities, anti-mitotic agents, alkylating agents, arsenic compounds, DNA topoisomerase inhibitors, taxanes, nucleoside analogues, plant alkaloids, and toxins; and synthetic derivatives thereof. Exemplary agents include, but are not limited to, alkylating agents: nitrogen mustards (e.g., cyclophosphamide, ifosfamide, trofosfamide, chlorambucil, estramustine, and melphalan), nitrosoureas (e.g., carmustine (BCNU) and lomustine (CCNU)), alkylsulphonates (e.g., busulfan and treosulfan), triazenes (e.g., dacarbazine, temozolomide), cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Similarly, additional exemplary agents including platinum-ontaining compounds (e.g., cisplatin, carboplatin, oxaliplatin), vinca alkaloids (e.g., vincristine, vinblastine, vindesine, and vinorelbine), taxoids (e.g., paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g., etoposide, etoposide phosphate, teniposide, topotecan, 9-aminocamptothecin, camptoirinotecan, irinotecan, crisnatol, mytomycin C), anti-metabolites, DHFR inhibitors (e.g., methotrexate, dichloromethotrexate, trimetrexate, edatrexate), IMP dehydrogenase inhibitors (e.g., mycophenolic acid, tiazofurin, ribavirin, and EICAR), ribonuclotide reductase inhibitors (e.g., hydroxyurea and deferoxamine), uracil analogs (e.g., 5-fluorouracil (5-FU), floxuridine, doxifluridine, ratitrexed, tegafur-uracil, capecitabine), cytosine analogs (e.g., cytarabine (ara C), cytosine arabinoside, and fludarabine), purine analogs (e.g., mercaptopurine and Thioguanine), Vitamin D3 analogs (e.g., EB 1089, CB 1093, and KH 1060), isoprenylation inhibitors (e.g., lovastatin), dopaminergic neurotoxins (e.g., 1-methyl-4-phenylpyridinium ion), cell cycle inhibitors (e.g., staurosporine), actinomycin (e.g., actinomycin D, dactinomycin), bleomycin (e.g., bleomycin A2, bleomycin B2, peplomycin), anthracycline (e.g., daunorubicin, doxorubicin, pegylated liposomal doxorubicin, idarubicin, epirubicin, pirarubicin, zorubicin, mitoxantrone), MDR inhibitors (e.g., verapamil), Ca²⁺ ATPase inhibitors (e.g., thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE®)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genentech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiment, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6 (5H)-phenanthridinone (Trevigen); benzamide (U.S. Pat. Re. 36,397); and NU1025 (Bowman et al.). The mechanism of action is generally related to the ability of PARP inhibitors to bind PARP and decrease its activity. PARP catalyzes the conversion of beta-nicotinamide adenine dinucleotide (NAD+) into nicotinamide and poly-ADP-ribose (PAR). Both poly(ADP-ribose) and PARP have been linked to regulation of transcription, cell proliferation, genomic stability, and carcinogenesis (Bouchard et. al. (2003) Exp. Hematol. 31:446-454); Herceg (2001) Mut. Res. 477:97-110). Poly(ADP-ribose) polymerase 1 (PARP1) is a key molecule in the repair of DNA single-strand breaks (SSBs) (de Murcia J. et al. (1997) Proc. Natl. Acad. Sci. U.S.A. 94:7303-7307; Schreiber et al. (2006) Nat. Rev. Mol. Cell Biol. 7:517-528; Wang et al. (1997) Genes Dev. 11:2347-2358). Knockout of SSB repair by inhibition of PARP1 function induces DNA double-strand breaks (DSBs) that may trigger synthetic lethality in cancer cells with defective homology-directed DSB repair (Bryant et al. (2005) Nature 434:913-917; Farmer et al. (2005) Nature 434:917-921). The foregoing examples of chemotherapeutic agents are illustrative and are not intended to be limiting.

In another embodiment, radiation therapy is used. The radiation used in radiation therapy may be ionizing radiation. Radiation therapy may also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy may be administered as external beam radiation or teletherapy wherein the radiation is directed from a remote source. The radiation treatment may also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In another embodiment, hormone therapy is used. Hormonal therapeutic treatments may comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In another embodiment, hyperthermia, a procedure in which body tissue is exposed to high temperatures (up to 106° F.) is used. Heat may help shrink tumors by damaging cells or depriving them of substances they need to live. Hyperthermia therapy may be local, regional, and whole-body hyperthermia, using external and internal heating devices. Hyperthermia is almost always used with other forms of therapy (e.g., radiation therapy, chemotherapy, and biological therapy) to try to increase their effectiveness. Local hyperthermia refers to heat that is applied to a very small area, such as a tumor. The area may be heated externally with high-frequency waves aimed at a tumor from a device outside the body. To achieve internal heating, one of several types of sterile probes may be used, including thin, heated wires or hollow tubes filled with warm water; implanted microwave antennae; and radiofrequency electrodes. In regional hyperthermia, an organ or a limb is heated. Magnets and devices that produce high energy are placed over the region to be heated. In another approach, called perfusion, some of the patient's blood is removed, heated, and then pumped (perfused) into the region that is to be heated internally. Whole-body heating is used to treat metastatic cancer that has spread throughout the body. It may be accomplished using warm-water blankets, hot wax, inductive coils (like those in electric blankets), or thermal chambers (similar to large incubators). Hyperthermia does not cause any marked increase in radiation side effects or complications. Heat applied directly to the skin, however, may cause discomfort or even significant local pain in about half the patients treated. It may also cause blisters, which generally heal rapidly.

In still another embodiment, photodynamic therapy (also called PDT, photoradiation therapy, phototherapy, or photochemotherapy) is used for the treatment of some types of cancer. It is based on the discovery that certain chemicals known as photosensitizing agents may kill one-celled organisms when the organisms are exposed to a particular type of light. PDT destroys cancer cells through the use of a fixed-frequency laser light in combination with a photosensitizing agent. In PDT, the photosensitizing agent is injected into the bloodstream and absorbed by cells all over the body. The agent remains in cancer cells for a longer time than it does in normal cells. When the treated cancer cells are exposed to laser light, the photosensitizing agent absorbs the light and produces an active form of oxygen that destroys the treated cancer cells. Light exposure must be timed carefully so that it occurs when most of the photosensitizing agent has left healthy cells but is still present in the cancer cells. The laser light used in PDT may be directed through a fiber-optic (a very thin glass strand). The fiber-optic is placed close to the cancer to deliver the proper amount of light. The fiber-optic may be directed through a bronchoscope into the lungs for the treatment of lung cancer or through an endoscope into the esophagus for the treatment of esophageal cancer. An advantage of PDT is that it causes minimal damage to healthy tissue. However, because the laser light currently in use cannot pass through more than about 3 centimeters of tissue (a little more than one and an eighth inch), PDT is mainly used to treat tumors on or just under the skin or on the lining of internal organs. Photodynamic therapy makes the skin and eyes sensitive to light for 6 weeks or more after treatment. Patients are advised to avoid direct sunlight and bright indoor light for at least 6 weeks. If patients must go outdoors, they need to wear protective clothing, including sunglasses. Other temporary side effects of PDT are related to the treatment of specific areas and may include coughing, trouble swallowing, abdominal pain, and painful breathing or shortness of breath. In December 1995, the U.S. Food and Drug Administration (FDA) approved a photosensitizing agent called porfimer sodium, or Photofrin®, to relieve symptoms of esophageal cancer that is causing an obstruction and for esophageal cancer that cannot be satisfactorily treated with lasers alone. In January 1998, the FDA approved porfimer sodium for the treatment of early nonsmall cell lung cancer in patients for whom the usual treatments for lung cancer are not appropriate. The National Cancer Institute and other institutions are supporting clinical trials (research studies) to evaluate the use of photodynamic therapy for several types of cancer, including cancers of the bladder, brain, larynx, and oral cavity.

In yet another embodiment, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors. The term “laser” stands for light amplification by stimulated emission of radiation. Ordinary light, such as that from a light bulb, has many wavelengths and spreads in all directions. Laser light, on the other hand, has a specific wavelength and is focused in a narrow beam. This type of high-intensity light contains a lot of energy. Lasers are very powerful and may be used to cut through steel or to shape diamonds. Lasers also may be used for very precise surgical work, such as repairing a damaged retina in the eye or cutting through tissue (in place of a scalpel). Although there are several different kinds of lasers, only three kinds have gained wide use in medicine: Carbon dioxide (CO₂) laser—This type of laser may remove thin layers from the skin's surface without penetrating the deeper layers. This technique is particularly useful in treating tumors that have not spread deep into the skin and certain precancerous conditions. As an alternative to traditional scalpel surgery, the CO₂ laser is also able to cut the skin. The laser is used in this way to remove skin cancers. Neodymium:yttrium-aluminum-garnet (Nd:YAG) laser—Light from this laser may penetrate deeper into tissue than light from the other types of lasers, and it may cause blood to clot quickly. It may be carried through optical fibers to less accessible parts of the body. This type of laser is sometimes used to treat throat cancers. Argon laser—This laser may pass through only superficial layers of tissue and is therefore useful in dermatology and in eye surgery. It also is used with light-sensitive dyes to treat tumors in a procedure known as photodynamic therapy (PDT). Lasers have several advantages over standard surgical tools, including: Lasers are more precise than scalpels. Tissue near an incision is protected, since there is little contact with surrounding skin or other tissue. The heat produced by lasers sterilizes the surgery site, thus reducing the risk of infection. Less operating time may be needed because the precision of the laser allows for a smaller incision. Healing time is often shortened; since laser heat seals blood vessels, there is less bleeding, swelling, or scarring. Laser surgery may be less complicated. For example, with fiber optics, laser light may be directed to parts of the body without making a large incision. More procedures may be done on an outpatient basis. Lasers may be used in two ways to treat cancer: by shrinking or destroying a tumor with heat, or by activating a chemical—known as a photosensitizing agent—that destroys cancer cells. In PDT, a photosensitizing agent is retained in cancer cells and may be stimulated by light to cause a reaction that kills cancer cells. CO₂ and Nd:YAG lasers are used to shrink or destroy tumors. They may be used with endoscopes, tubes that allow physicians to see into certain areas of the body, such as the bladder. The light from some lasers may be transmitted through a flexible endoscope fitted with fiber optics. This allows physicians to see and work in parts of the body that could not otherwise be reached except by surgery and therefore allows very precise aiming of the laser beam. Lasers also may be used with low-power microscopes, giving the doctor a clear view of the site being treated. Used with other instruments, laser systems may produce a cutting area as small as 200 microns in diameter—less than the width of a very fine thread. Lasers are used to treat many types of cancer. Laser surgery is a standard treatment for certain stages of glottis (vocal cord), cervical, skin, lung, vaginal, vulvar, and penile cancers. In addition to its use to destroy the cancer, laser surgery is also used to help relieve symptoms caused by cancer (palliative care). For example, lasers may be used to shrink or destroy a tumor that is blocking a patient's trachea (windpipe), making it easier to breathe. It is also sometimes used for palliation in colorectal and anal cancer. Laser-induced interstitial thermotherapy (LITT) is one of the most recent developments in laser therapy. LITT uses the same idea as a cancer treatment called hyperthermia; that heat may help shrink tumors by damaging cells or depriving them of substances they need to live. In this treatment, lasers are directed to interstitial areas (areas between organs) in the body. The laser light then raises the temperature of the tumor, which damages or destroys cancer cells.

In one aspect, provided herein is a method of eliciting in a subject an immune response to a cell that expresses MAGEA1. In some embodiments, the method comprises administering to the subject a pharmaceutical composition described herein, wherein the pharmaceutical composition, when administered to the subject, elicits an immune response to the cell that expresses MAGEA1.

In some embodiments, the immune response can include a cell-mediated immune response. A cellular immune response is a response that involves T cells and may be determined in vitro, ex vivo, or in vivo. For example, a general cellular immune response may be determined as the T cell proliferative activity in cells (e.g., peripheral blood leukocytes (PBLs)) sampled from the subject at a suitable time following the administering of a pharmaceutical composition. Following incubation of e.g., PBMCs with a stimulator for an appropriate period, [³H]thymidine incorporation may be determined. The subset of T cells that is proliferating may be determined using flow cytometry.

In another aspect encompassed by the present invention, the methods provided herein include administering to both human and non-human mammals as described above. Veterinary applications also are contemplated. In some embodiments, the subject may be any living organism in which an immune response may be elicited.

In some embodiments, the pharmaceutical composition may be administered at any time that is appropriate. For example, the administering may be conducted before or during treatment of a subject having a disorder characterized by MAGEA1 expression (e.g., a non-malignant disorder, a hyperproliferative disorder, or a relapse of a hyperproliferative disorder characterized by MAGEA1 expression), and continued after the disorder characterized by MAGEA1 expression becomes clinically undetectable. The administering also may be continued in a subject showing signs of recurrence.

In some embodiments, the pharmaceutical composition may be administered in a therapeutically or a prophylactically effective amount. Administering the pharmaceutical composition to the subject may be carried out using known procedures, and at dosages and for periods of time sufficient to achieve a desired effect.

In some embodiments, the pharmaceutical composition may be administered to the subject at any suitable site. Administration may be accomplished using methods generally known in the art. Agents, including cells, may be introduced to the desired site by direct injection, or by any other means used in the art including, but are not limited to, intravascular, intracerebral, parenteral, intraperitoneal, intravenous, epidural, intraspinal, intrasternal, intraarticular, intra-synovial, intrathecal, intra-arterial, intracardiac, or intramuscular administration. For example, subjects of interest may be engrafted with the transplanted cells by various routes. Such routes include, but are not limited to, intravenous administration, subcutaneous administration, administration to a specific tissue (e.g., focal transplantation), injection into the femur bone marrow cavity, injection into the spleen, administration under the renal capsule of fetal liver, and the like. In certain embodiment, the cancer vaccine encompassed by the present invention is injected to the subject intratumorally or subcutaneously. Cells may be administered in one infusion, or through successive infusions over a defined time period sufficient to generate a desired effect. Exemplary methods for transplantation, engraftment assessment, and marker phenotyping analysis of transplanted cells are well-known in the art (see, for example, Pearson et al. (2008) Curr. Protoc. Immunol. 81:15.21.1-15.21.21; Ito et al. (2002) Blood 100:3175-3182; Traggiai et al. (2004) Science 304:104-107; Ishikawa et al. Blood (2005) 106:1565-1573; Shultz et al. (2005) J. Immunol. 174:6477-6489; and Holyoake et al. (1999) Exp. Hematol. 27:1418-1427).

In some embodiments, the dose may be administered in an amount and for a period of time effective in bringing about a desired response, be it eliciting the immune response or the prophylactic or therapeutic treatment of a disorder characterized by MAGEA1 expression (e.g., a non-malignant disorder, a hyperproliferative disorder, or a relapse of a hyperproliferative disorder characterized by MAGEA1 expression) and/or symptoms associated therewith.

The pharmaceutical composition may be given subsequent to, preceding, or contemporaneously with other therapies including therapies that also elicit an immune response in the subject. For example, the subject may previously or concurrently be treated by other forms of immunomodulatory agents, such other therapies may be provided in such a way so as not to interfere with the immunogenicity of the compositions described herein.

Administering may be properly timed by the care giver (e.g., physician, veterinarian), and may depend on the clinical condition of the subject, the objectives of administering, and/or other therapies also being contemplated or administered. In some embodiments, an initial dose may be administered, and the subject monitored for an immunological and/or clinical response. Suitable means of immunological monitoring include using patient's peripheral blood lymphocyte (PBL) as responders and immunogenic peptides or peptide-MHC complexes described herein as stimulators. An immunological reaction also may be determined by a delayed inflammatory response at the site of administering. One or more doses subsequent to the initial dose may be given as appropriate, typically on a monthly, semimonthly, or a weekly basis, until the desired effect is achieved. Thereafter, additional booster or maintenance doses may be given as required, particularly when the immunological or clinical benefit appears to subside.

In general, an appropriate dosage and treatment regimen provides the active molecules or cells in an amount sufficient to provide a benefit. Such a response may be monitored by establishing an improved clinical outcome (e.g., more frequent remissions, complete or partial, or longer disease-free survival) in treated subjects as compared to non-treated subjects. Increases in preexisting immune responses to a viral protein generally correlate with an improved clinical outcome. Such immune responses may generally be evaluated using standard proliferation, cytotoxicity or cytokine assays, which are routine.

For prophylactic use, a dose should be sufficient to prevent, delay the onset of, or diminish the severity of a disease associated with disease or disorder. Prophylactic benefit of the immunogenic compositions administered according to the methods described herein can be determined by performing pre-clinical (including in vitro, ex vivo, and in vivo animal studies) and clinical studies and analyzing data obtained therefrom by appropriate statistical, biological, and clinical methods and techniques, all of which can readily be practiced by an ordinarily skilled artisan.

As used herein, administration of a composition refers to delivering the same to a subject, regardless of the route or mode of delivery. Administration may be effected continuously or intermittently, and parenterally. Administration may be for treating a subject already confirmed as having a recognized condition, disease or disease state, or for treating a subject susceptible to or at risk of developing such a condition, disease or disease state. Co-administration with an adjunctive therapy may include simultaneous and/or sequential delivery of multiple agents in any order and on any dosing schedule (e.g., engineered immune cells with one or more cytokines; immunosuppressive therapy such as calcineurin inhibitors, corticosteroids, microtubule inhibitors, low dose of a mycophenolic acid prodrug, or any combination thereof).

In some embodiments, a plurality of doses of a host cell (e.g., an engineered immune cell) described herein is administered to the subject, which may be administered at intervals between administrations of about two to about four weeks.

Treatment or prevention methods encompassed by the present invention may be administered to a subject as part of a treatment course or regimen, which may comprise additional treatments prior to, or after, administration of the instantly disclosed unit doses, cells, or compositions. For example, in some embodiments, a subject receiving a unit dose of the host cell (e.g., an engineered immune cell) is receiving or had previously received a hematopoietic cell transplant (HCT; including myeloablative and non-myeloablative HCT). In any of the foregoing embodiments, a hematopoietic cell used in an HCT may be a “universal donor” cell that is modified to reduce or eliminate expression of one or more endogenous genes that encode a polypeptide product selected from an MHC, antigen, and a binding protein (e.g., by a chromosomal gene knockout according to the methods described herein).

Techniques and regimens for performing cell transplantation are known in the art and may comprise transplantation of any suitable donor cell, such as a cell derived from umbilical cord blood, bone marrow, or peripheral blood, a hematopoietic stem cell, a mobilized stem cell, or a cell from amniotic fluid. Accordingly, in some embodiments, a host cell (e.g., an engineered immune cell) encompassed by the present invention may be administered with or shortly after stem cell therapy.

Methods encompassed by the present invention may, in some embodiments, further include administering one or more additional agents to treat the disease or disorder (e.g., a disorder characterized by MAGEA1 expression such as a non-malignant disorder, a hyperproliferative disorder, or a relapse of a hyperproliferative disorder characterized by MAGEA1 expression)) in a combination therapy. For example, in some embodiments, a combination therapy comprises administering host cell or binding protein encompassed by the present invention with (concurrently, simultaneously, or sequentially) an antiviral agent. In some embodiments, a combination therapy comprises administering a host cell or binding protein encompassed by the present invention with lopinavir/ritonavir, chloroquine, ribavirin, steroid drugs, hydroxychloroquine, and/or interferon α. In some embodiments, a combination therapy comprises administering a host cell, composition, or unit dose of the host cells encompassed by the present invention with a secondary therapy, such as a surgery, an antibody, a vaccine, or any combination thereof.

In some embodiments, the subject is a human, such as a human with a disorder characterized by MAGEA1 expression (e.g., a non-malignant disorder, a hyperproliferative disorder, or a relapse of a hyperproliferative disorder characterized by MAGEA1 expression). In some embodiments, the subject is a rodent, such as a mouse. In some such embodiments, the mouse is a transgenic mouse, such as a mouse expressing human MHC (i.e., HLA) molecules, such as HLA-A2 (e.g., Nicholson et al. (2012) Adv. Hematol. 2012:404081).

In some embodiments, the subject is a transgenic mouse expressing human TCRs or is an antigen-negative mouse (e.g., Li et al. (2010) Nat. Med. 16:1029-1034 and Obenaus et al. (2015) Nat. Biotechnol. 33:402-407). In some embodiments, the subject is a transgenic mouse expressing human HLA molecules and human TCRs.

In some embodiments, such as where the subject is a transgenic HLA mouse, the identified TCRs are modified, e.g., to be chimeric or humanized. In some embodiments, the TCR scaffold is modified, such as analogous to known binding protein humanizing methods.

c. Screening Methods

Another aspect encompassed by the present invention encompasses screening assays.

In some embodiments, methods are provided for selecting agents that bind to a MAGEA1 immunogenic peptide or pMHC described herein. For example, a method of identifying a peptide-binding molecule, or antigen-binding fragment thereof, that binds to a peptide epitope selected from the peptide sequences listed in Table 1 comprising a) providing a cell presenting a peptide epitope selected from the peptide sequences listed in Table 1 in the context of an MHC molecule on the surface of the cell; b) determining binding of a plurality of candidate peptide-binding molecules or antigen-binding fragments thereof to the peptide epitope in the context of the MHC molecule on the cell; and c) identifying one or more peptide-binding molecules or antigen-binding fragments thereof that bind to the peptide epitope in the context of the MHC molecule, is provided.

In some embodiments, a method of identifying a peptide-binding molecule or antigen-binding fragment thereof that binds to a peptide epitope selected from the peptide sequences listed in Table 1 comprising: a) providing a peptide epitope either alone or in a stable MHC-peptide complex, comprising a peptide epitope selected from the peptide sequences listed in Table 1, either alone or in the context of an MHC molecule; b) determining binding of a plurality of candidate peptide-binding molecules or antigen-binding fragments thereof to the peptide or stable MHC-peptide complex; and c) identifying one or more peptide-binding molecules or antigen-binding fragments thereof that bind to the peptide epitope or the stable MHC-peptide complex, optionally wherein the MHC or MHC-peptide complex is as described herein, is provided.

In some embodiments, provide herein are methods of identifying a peptide-binding molecule or antigen-binding fragment thereof that binds to a peptide epitope selected from Table 1.

In some embodiments, the peptide binding molecule (e.g., MHC-peptide binding molecule) is a molecule or portion thereof that possesses the ability to bind (e.g., specifically and/or selectively) to a peptide epitope that is presented or displayed in the context of an MHC molecule (MHC-peptide complex), such as on the surface of a cell. Exemplary peptide binding molecules include T cell receptors or antibodies, or antigen-binding portions thereof, including single chain immunoglobulin variable regions (e.g., scTCR, scFv) thereof, that exhibit specific ability to bind to an MHC-peptide complex. In some embodiments, the peptide binding molecule is a TCR or antigen-binding fragment thereof. In some embodiments, the peptide binding molecule is an antibody, such as a TCR-like antibody or antigen-binding fragment thereof. In some embodiments, the peptide binding molecule is a TCR-like CAR that contains an antibody or antigen binding fragment thereof, such as a TCR-like antibody, such as one that has been engineered to bind to MHC-peptide complexes. In some embodiments, the peptide binding molecule may be derived from natural sources, or it may be partly or wholly synthetically or recombinantly produced.

In some embodiments, a binding molecule that binds to a peptide epitope may be identified by contacting one or more candidate peptide binding molecules, such as one or more candidate TCR molecules, antibodies, or antigen-binding fragments thereof, with an MHC-peptide complex, and assessing whether each of the one or more candidate binding molecules binds (e.g., specifically and/or selectively) to the MHC-peptide complex. The methods may be performed in vitro, ex vivo, or in vivo. Methods are well-known in the art for screening, such as described in U.S. Pat. Publ. 2020/0102553.

In some embodiments, the methods include contacting a plurality or library of binding molecules, such as a plurality or library of TCRs or antibodies, with an MHC-restricted epitope and identifying or selecting molecules that specifically and/or selectively bind such an epitope. In some embodiments, a library or collection containing a plurality of different binding molecules, such as a plurality of different TCRs or a plurality of different antibodies, may be screened or assessed for binding to an MHC-restricted epitope. In some embodiments, such as for selecting a binding protein that specifically and/or selectively binds an MHC-restricted peptide, hybridoma methods may be employed.

In some embodiments, screening methods may be employed in which a plurality of candidate binding molecules, such as a library or collection of candidate binding molecules, are individually contacted with an peptide binding molecule, either simultaneously or sequentially. Library members that specifically and/or selectively bind to a particular MHC-peptide complex may be identified or selected. In some embodiments, the library or collection of candidate binding molecules may contain at least 2, 5, 10, 100, 10³, 10⁴, 10⁵, 10⁶, 10⁷, 10⁸, 10⁹, or more different peptide binding molecules.

In some embodiments, the methods may be employed to identify a peptide binding molecule, such as a TCR or an antibody, that exhibits binding for more than one MHC haplotype or more than one MHC allele. In some embodiments, the peptide binding molecule, such as a TCR or antibody, specifically and/or selectively binds or recognizes a peptide epitope presented in the context of a plurality of MHC class I haplotypes or alleles. In some embodiments, the peptide binding molecule, such as a TCR or antibody, specifically and/or selectively binds or recognizes a peptide epitope presented in the context of a plurality of MHC class II haplotypes or alleles.

A variety of assays are known for assessing binding affinity and/or determining whether a binding molecule specifically and/or selectively binds to a particular ligand (e.g., MHC-peptide complex). It is within the level of a skilled artisan to determine the binding affinity of a TCR for a T cell epitope of a target polypeptide, such as by using any of a number of binding assays that are well-known in the art. For example, in some embodiments, a Biacore® machine may be used to determine the binding constant of a complex between two proteins. The dissociation constant (K_{D}) for the complex may be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy and surface plasmon resonance (Biacore®) analysis (see, e.g., Scatchard et al. (1949) Ann. N.Y. Acad. Sci. 51:660; Wilson (2002) Science 295:2103; Wolff et al. (1993) Cancer Res. 53:2560; and U.S. Pat. Nos. 5,283,173, 5,468,614, or the equivalent), flow cytometry, sequencing and other methods for detection of expressed nucleic acids. In one example, apparent affinity for a TCR is measured by assessing binding to various concentrations of tetramers, for example, by flow cytometry using labeled tetramers. In one example, apparent K_{D} of a TCR is measured using 2-fold dilutions of labeled tetramers at a range of concentrations, followed by determination of binding curves by non-linear regression, apparent K_{D} being determined as the concentration of ligand that yielded half-maximal binding.

In some embodiments, the methods may be used to identify binding molecules that bind only if the particular peptide is present in the complex, and not if the particular peptide is absent or if another, non-overlapping or unrelated peptide is present. In some embodiments, the binding molecule does not substantially bind the MHC in the absence of the bound peptide, and/or does not substantially bind the peptide in the absence of the MHC. In some embodiments, the binding molecules are at least partially specific. In some embodiments, an exemplary identified binding molecule may bind to an MHC-peptide complex if the particular peptide is present, and also bind if a related peptide that has one or two substitutions relative to the particular peptide is present.

In some embodiments, an identified antibody, such as a TCR-like antibody, may be used to produce or generate a chimeric antigen receptors (CARs) containing a non-TCR antibody that specifically and/or selectively binds to a MHC-peptide complex.

In some embodiments, the methods of identifying a peptide binding molecule, such as a TCR or TCR-like antibody or TCR-like CAR, may be used to engineer cells expressing or containing a peptide binding molecule. In some embodiments, a cell or engineered cell is a T cell. In some embodiments, the T cell is a CD4+ or CD8+ T cell. In some embodiments, the peptide binding molecule recognizes a MHC class I peptide complex, an MHC class II peptide complex and/or an MHC-E peptide complex. In some embodiments, a peptide binding molecule, such as a TCR or antibody or CAR, that specifically and/or selectively recognizes a peptide in the context of an MHC class I may be used to engineer CD8+ T cells. In some embodiments, also provided is a composition of engineered CD8+ T cells expressing or containing the TCR, antibody or CAR, for recognition of a peptide presented in the context of MHC class I. In any of such embodiments, the cells may be used in methods of adoptive cell therapy.

In some embodiments, TCR libraries may be generated by amplification of the repertoire of Vα and Vβ from T cells isolated from a subject, including cells present in PBMCs, spleen or other lymphoid organ. In some cases, T cells may be amplified from tumor-infiltrating lymphocytes (TILs). In some embodiments, TCR libraries may be generated from CD4+ or CD8+ cells. In some embodiments, the TCRs may be amplified from a T cell source of a normal of healthy subject, i.e., normal TCR libraries. In some embodiments, the TCRs may be amplified from a T cell source of a diseased subject, i.e., diseased TCR libraries. In some embodiments, degenerate primers are used to amplify the gene repertoire of V_{α} and VP, such as by RT-PCR in samples, such as T cells, obtained from humans. In some embodiments, scTv libraries may be assembled from naive Vα and Vβ libraries in which the amplified products are cloned or assembled to be separated by a linker. Depending on the source of the subject and cells, the libraries may be HLA allele-specific.

Alternatively, in some embodiments, TCR libraries may be generated by mutagenesis or diversification of a parent or scaffold TCR molecule. For example, in some aspects, a subject, e.g., human or other mammal such as a rodent, may be vaccinated with a peptide, such as a peptide identified by the present methods. In some embodiments, a sample may be obtained from the subject, such as a sample containing blood lymphocytes. In some instances, binding molecules, e.g., TCRs, may be amplified out of the sample, e.g., T cells contained in the sample. In some embodiments, antigen-specific T cells may be selected, such as by screening to assess CTL activity against the peptide. In some aspects, TCRs, e.g., present on the antigen-specific T cells, may be selected, such as by binding activity, e.g., particular affinity or avidity for the antigen. In some aspects, the TCRs are subjected to directed evolution, such as by mutagenesis, e.g., of the α or β chain. In some aspects, particular residues within CDRs of the TCR are altered. In some embodiments, selected TCRs may be modified by affinity maturation. In some aspects, a selected TCR may be used as a parent scaffold TCR against the antigen.

In some embodiments, the subject is a human, such as a human with a disorder characterized by MAGEA1 expression. In some embodiments, the subject is a rodent, such as a mouse. In some such embodiments, the mouse is a transgenic mouse, such as a mouse expressing human MHC (i.e., HLA) molecules, such as HLA-A2 (e.g., Nicholson et al. (2012) Adv. Hematol. 2012:404081).

In some embodiments, the subject is a transgenic mouse expressing human TCRs or is an antigen-negative mouse (e.g., Li et al. (2010) Nat Med. 161029-1034; Obenaus et al. (2015) Nat. Biotechnol. 33:402-407). In some embodiments, the subject is a transgenic mouse expressing human HLA molecules and human TCRs.

In some embodiments, such as where the subject is a transgenic HLA mouse, the identified TCRs are modified, e.g., to be chimeric or humanized. In some aspects, the TCR scaffold is modified, such as analogous to known antibody humanizing methods.

In some embodiments, such a scaffold molecule is used to generate a library of TCRs.

For example, in some embodiments, the library includes TCRs or antigen-binding portions thereof that have been modified or engineered compared to the parent or scaffold TCR molecule. In some embodiments, directed evolution methods may be used to generate TCRs with altered properties, such as with higher affinity for a specific MHC-peptide complex. In some embodiments, display approaches involve engineering, or modifying, a known, parent or reference TCR. For example, in some cases, a wild-type TCR may be used as a template for producing mutagenized TCRs in which in one or more residues of the CDRs are mutated, and mutants with an desired altered property, such as higher affinity for a desired target antigen, are selected. In some embodiments, directed evolution is achieved by display methods including, but not limited to, yeast display (Holler et al. (2003) Nat. Immunol. 4:55-62; Holler et al. (2000) Proc. Natl. Acad. Sci. U.S.A. 97:5387-5392), phage display (Li et al. (2005) Nat. Biotechnol. 23:349-354), or T cell display (Chervin et al. (2008) J. Immunol. Methods 339:175-184).

In some embodiments, the libraries may be soluble. In some embodiments, the libraries are display libraries in which the TCR is displayed on the surface of a phage or cell, or attached to a particle or molecule, such as a cell, ribosome or nucleic acid, e.g., RNA or DNA. Typically, the TCR libraries, including normal and disease TCR libraries or diversified libraries, may be generated in any form, including as a heterodimer or as a single chain form. In some embodiments, one or more members of the TCR may be a two-chain heterodimer. In some embodiments, pairing of the Vα and Vβ chains may be promoted by introduction of a disulfide bond. In some embodiments, members of the TCR library may be a TCR single chain (scTv or ScTCR), which, in some cases, may include a Vα and Vβ chain separated by a linker. Further, in some cases, upon screening and selection of a TCR from the library, the selected member may be generated in any form, such as a full-length TCR heterodimer or single-chain form or as antigen-binding fragments thereof.

Other methods of identifying molecules that bind to a peptide in the context of an MHC molecule are also described in U.S. Pat. Appl. No. 2020/0182884.

More generally, the present invention encompasses assays for screening agents, such as test proteins, that bind to, or modulate the activity of, MAGEA1 or an antigen thereof. Such agents include, without limitation, antibodies, proteins, fusion proteins, small molecules, and nucleic acids. In some embodiments, a method for identifying an agent which modulates an immune response entails determining the ability of the candidate agent to modulate MAGEA1 activity and further modulate an immune response of interest, such as modulated cytotoxic T cell activation and/or activity, sensitivity of cancer cells to immune checkpoint therapy, and the like.

In some embodiments, an assay is a cell-free or cell-based assay, comprising contacting a target, with a test agent, and determining the ability of the test agent to modulate (e.g., upregulate or downregulate) the amount and/or activity of the target, such as by measuring direct or indirect parameters as described below.

In some embodiments, an assay is a cell-based assay, such as one comprising contacting (a) a cell of interest with a test agent and determining the ability of the test agent to modulate the amount and/or activity of the target, such as binding characteristics. Determining the ability of the polypeptides to bind to, or interact with, each other may be accomplished, e.g., by measuring direct binding or by measuring a parameter of immune cell activation or function.

In another embodiment, an assay is a cell-based assay, comprising contacting a cell such as a cancer cell with immune cells (e.g., cytotoxic T cells) and a test agent, and determining the ability of the test agent to modulate the amount and/or activity of the target, and/or modulated immune responses, such as by measuring direct or indirect parameters as described below.

The methods described above and herein may also be adapted to test one or more agents that are already known to modulate the amount and/or activity of one or more biomarkers described herein to confirm modulation of the one or more biomarkers and/or to confirm the effects of the agents on readouts of a desired phenotype, such as modulated immune responses, sensitivity to immune checkpoint blockade, and the like.

In a direct binding assay, biomarker protein (or their respective target polypeptides or molecules) may be coupled with a radioisotope or enzymatic label such that binding may be determined by detecting the labeled protein or molecule in a complex. For example, the targets may be labeled with ¹²⁵I, ³⁵S, ¹⁴C, or ³H, either directly or indirectly, and the radioisotope detected by direct counting of radioemmission or by scintillation counting. Alternatively, the targets may be enzymatically labeled with, for example, horseradish peroxidase, alkaline phosphatase, or luciferase, and the enzymatic label detected by determination of conversion of an appropriate substrate to product. Determining the interaction between target and substrate may also be accomplished using standard binding or enzymatic analysis assays. In one or more embodiments of the above described assay methods, it may be desirable to immobilize polypeptides or molecules to facilitate separation of complexed from uncomplexed forms of one or both of the proteins or molecules, as well as to accommodate automation of the assay.

Binding of a test agent to a target may be accomplished in any vessel suitable for containing the reactants. Non-limiting examples of such vessels include microtiter plates, test tubes, and micro-centrifuge tubes. Immobilized forms of the antibodies encompassed by the present invention may also include antibodies bound to a solid phase like a porous, microporous (with an average pore diameter less than about one micron) or macroporous (with an average pore diameter of more than about 10 microns) material, such as a membrane, cellulose, nitrocellulose, or glass fibers; a bead, such as that made of agarose or polyacrylamide or latex; or a surface of a dish, plate, or well, such as one made of polystyrene.

For example, in a direct binding assay, the polypeptides may be coupled with a radioisotope or enzymatic label such that polypeptide interactions and/or activity, such as binding events, may be determined by detecting the labeled protein in a complex. For example, the polypeptides may be labeled with ¹²⁵I, ³⁵S, ¹⁴C, or ³H, either directly or indirectly, and the radioisotope detected by direct counting of radioemmission or by scintillation counting. Alternatively, the polypeptides may be enzymatically labeled with, for example, horseradish peroxidase, alkaline phosphatase, or luciferase, and the enzymatic label detected by determination of conversion of an appropriate substrate to product.

It is also within the scope of the present invention to determine the ability of an agent to modulate a parameter of interest without the labeling of any of the interactants. For example, a microphysiometer may be used to detect interaction between polypeptides without the labeling of polypeptides to be monitored (McConnell et al. (1992) Science 257:1906-1912). As used herein, a “microphysiometer” (e.g., Cytosensor®) is an analytical instrument that measures the rate at which a cell acidifies its environment using a light-addressable potentiometric sensor (LAPS). Changes in this acidification rate may be used as an indicator of the interaction between compound and receptor.

In some embodiments, determining the ability of a test agent (e.g. antibodies, fusion proteins, peptides, or small molecules) to modulate the interaction between a given set of polypeptides may be accomplished by determining the activity of one or more members of the set of polypeptides. For example, the activity of a protein and/or one or more binding partners may be determined by detecting induction of a cellular second messenger (e.g., intracellular signaling), detecting catalytic/enzymatic activity of an appropriate substrate, detecting the induction of a reporter gene (comprising a target-responsive regulatory element operatively linked to a nucleic acid encoding a detectable marker, e.g., chloramphenicol acetyl transferase), or detecting a cellular response regulated by the protein and/or the one or more binding partners. Determining the ability of the test agent to bind to or interact with said polypeptide may be accomplished, for example, by measuring the ability of a compound to modulate immune cell costimulation or inhibition in a proliferation assay, or by interfering with the ability of said polypeptide to bind to antibodies that recognize a portion thereof.

Agents that modulate target amount and/or activity, such as interactions with one or more binding partners, may be identified by their ability to inhibit immune cell proliferation, and/or effector function, or to induce anergy, clonal deletion, and/or exhaustion when added to an in vitro assay. For example, cells may be cultured in the presence of an agent that stimulates signal transduction via an activating receptor. A number of recognized readouts of cell activation may be employed to measure, cell proliferation or effector function (e.g., antibody production, cytokine production, phagocytosis) in the presence of the agent. The ability of a test agent to block this activation may be readily determined by measuring the ability of the agent to effect a decrease in proliferation or effector function being measured, using techniques known in the art.

For example, agents encompassed by the present invention may be tested for the ability to inhibit or enhance costimulation in a T cell assay, as described in Freeman et al. (2000) J. Exp. Med. 192:1027 and Latchman et al. (2001) Nat. Immunol. 2:261. CD4+ T cells may be isolated from human PBMCs and stimulated with activating anti-CD3 antibody. Proliferation of T cells may be measured by 3H thymidine incorporation. An assay may be performed with or without CD28 costimulation in the assay. Similar assays may be performed with Jurkat T cells and PHA-blasts from PBMCs.

Alternatively, agents encompassed by the present invention may be tested for the ability to modulate cellular production of cytokines which are produced by or whose production is enhanced or inhibited in immune cells in response to modulation of the one or more biomarkers. Indicative cytokines released by immune cells of interest may be identified by ELISA or by the ability of an antibody which blocks the cytokine to inhibit immune cell proliferation or proliferation of other cell types that is induced by the cytokine, such as those described in the Examples section. An in vitro immune cell costimulation assay may also be used in a method for identifying cytokines which may be modulated by modulation of the one or more biomarkers. For example, if a particular activity induced upon costimulation, e.g., immune cell proliferation, cannot be inhibited by addition of blocking antibodies to known cytokines, the activity may result from the action of an unknown cytokine. Following costimulation, this cytokine may be purified from the media by conventional methods and its activity measured by its ability to induce immune cell proliferation. To identify cytokines which may play a role the induction of tolerance, an in vitro T cell costimulation assay as described above may be used. In this case, T cells would be given the primary activation signal and contacted with a selected cytokine, but would not be given the costimulatory signal. After washing and resting the immune cells, the cells would be rechallenged with both a primary activation signal and a costimulatory signal. If the immune cells do not respond (e.g., proliferate or produce cytokines) they have become tolerized and the cytokine has not prevented the induction of tolerance. However, if the immune cells respond, induction of tolerance has been prevented by the cytokine. Those cytokines which are capable of preventing the induction of tolerance may be targeted for blockage in vivo in conjunction with reagents which block B lymphocyte antigens as a more efficient means to induce tolerance in transplant recipients or subjects with autoimmune diseases.

In some embodiments, an assay encompassed by the present invention is a cell-free assay for screening for agents that modulate the interaction between a biomarker and/or one or more binding partners, comprising contacting a polypeptide and one or more natural binding partners, or biologically active portion thereof, with a test agent and determining the ability of the test compound to modulate the interaction between the polypeptide and one or more natural binding partners, or biologically active portion thereof. Binding of the test compound may be determined either directly or indirectly as described above. In one embodiment, the assay includes contacting the polypeptide, or biologically active portion thereof, with its binding partner to form an assay mixture, contacting the assay mixture with a test compound, and determining the ability of the test agent to interact with the polypeptide in the assay mixture, wherein determining the ability of the test agent to interact with the polypeptide comprises determining the ability of the test agent to preferentially bind to the polypeptide or biologically active portion thereof, as compared to the binding partner.

In some embodiments, whether for cell-based or cell-free assays, a test agent may further be assayed to determine whether it affects binding and/or activity of the interaction between the polypeptide and the one or more binding partners, with other binding partners. Other useful binding analysis methods include the use of real-time Biomolecular Interaction Analysis (BIA) (Sjolander and Urbaniczky (1991) Anal. Chem. 63:2338-2345 and Szabo et al. (1995) Curr. Opin. Struct. Biol. 5:699-705). As used herein, “BIA” is a technology for studying biospecific interactions in real time, without labeling any of the interactants (e.g., Biacore®). Changes in the optical phenomenon of surface plasmon resonance (SPR) may be used as an indication of real-time reactions between biological polypeptides. Polypeptides of interest may be immobilized on a Biacore® chip and multiple agents (blocking antibodies, fusion proteins, peptides, or small molecules) may be tested for binding to the polypeptide of interest. An example of using the BIA technology is described by Fitz et al. (1997) Oncogene 15:613.

The cell-free assays encompassed by the present invention are amenable to use of both soluble and/or membrane-bound forms of proteins. In the case of cell-free assays in which a membrane-bound form protein is used it may be desirable to utilize a solubilizing agent such that the membrane-bound form of the protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether) n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In one or more embodiments of the above described assay methods, it may be desirable to immobilize either polypeptides to facilitate separation of complexed from uncomplexed forms of one or both of the proteins, as well as to accommodate automation of the assay. Binding of a test compound to a polypeptide, may be accomplished in any vessel suitable for containing the reactants. Examples of such vessels include microtiter plates, test tubes, and micro-centrifuge tubes. In one embodiment, a fusion protein may be provided which adds a domain that allows one or both of the proteins to be bound to a matrix. For example, glutathione-S-transferase-based polypeptide fusion proteins, or glutathione-S-transferase/target fusion proteins, may be adsorbed onto glutathione sepharose beads (Sigma Chemical, St. Louis, MO) or glutathione derivatized microtiter plates, which are then combined with the test compound, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes may be dissociated from the matrix, and the level of polypeptide binding or activity determined using standard techniques.

The present invention further pertains to novel agents identified by the above-described screening assays. Accordingly, it is within the scope of the present invention to further use an agent identified as described herein in an appropriate model system. For example, an agent identified as described herein may be used in a model system to determine the efficacy, toxicity, or side effects of treatment with such an agent. Alternatively, an agent identified as described herein may be used in a model system to determine the mechanism of action of such an agent. Furthermore, the present invention pertains to uses of novel agents identified by the above-described screening assays for treatments as described herein.

d. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect encompassed by the present invention encompasses diagnostic assays for determining (e.g., detecting) the presence, absence, amount, and/or activity level of MAGEA1 or reactivity to MAGEA1 in the context of a biological sample (e.g., blood, serum, cells, or tissue) to thereby determine whether an individual afflicted with a disorder characterized by MAGEA1 expression is likely to respond to therapy, whether in an original state or as a recurrence. Such assays may be used for prognostic or predictive purpose to thereby prophylactically treat an individual prior to the onset or after recurrence of a disorder characterized by MAGEA1 expression.

The diagnostic methods described herein may furthermore be utilized to identify subjects having or at risk of developing a disorder associated with expression or lack thereof of MAGEA1. As used herein, the term “aberrant” includes an upregulation or downregulation of MAGEA1 from normal levels. Aberrant expression or activity includes increased or decreased expression or activity, as well as expression or activity which does not follow the normal developmental pattern of expression or the subcellular pattern of expression. For example, aberrant levels is intended to include the cases in which a mutation in the biomarker gene or regulatory sequence, or amplification of the chromosomal gene, thereof causes upregulation or downregulation of the biomarker of interest. As used herein, the term “unwanted” includes an unwanted phenomenon involved in a biological response, such as immune cell activity.

The assays described herein, such as the preceding diagnostic assays or the following assays, may be utilized to identify a subject having or at risk of developing a disorder associated with MAGEA1 misregulation. Thus, the present invention provides a method for identifying a disorder associated with aberrant or unwanted MAGEA1 regulation in which a test sample is obtained from a subject and MAGEA1 expression is detected, wherein the presence of MAGEA1 expression is diagnostic for a subject having or at risk of developing the disorder associated with aberrant or unwanted MAGEA1 expression. As used herein, a “test sample” refers to a biological sample obtained from a subject of interest. For example, a test sample may be a biological fluid (e.g., cerebrospinal fluid or serum), cell sample, or tissue, such as a histopathological slide of the tumor microenvironment, peritumoral area, and/or intratumoral area.

Furthermore, the prognostic assays described herein may be used to determine whether a subject may be administered an agent described herein to treat such a disorder associated with aberrant or unwanted MAGEA1 expression. For example, such methods may be used to determine whether a subject may be effectively treated with one or a combination of agents. Thus, the present invention provides methods for determining whether a subject may be effectively treated with one or more agents described herein for treating a disorder associated with aberrant or unwanted MAGEA1 expression.

The methods described herein may be performed, for example, by utilizing pre-packaged diagnostic kits comprising at least one antibody reagent described herein, which may be conveniently used, e.g., in clinical settings to diagnose patients exhibiting symptoms or family history of a disease or illness involving the biomarker of interest.

Furthermore, any cell type or tissue in which the biomarker of interest is expressed may be utilized in the prognostic assays described herein.

e. Monitoring of Effects During Clinical Trials

Monitoring the influence of a disorder characterized by MAGEA1 expression therapy (e.g., compounds, drugs, vaccines, cell therapies, and the like) on immune responses, such as T cell reactivity (e.g., the presence of binding and/or T cell activation and/or effector function), may be applied not only in basic candidate MAGEA1 antigen binding molecule screening, but also in clinical trials. For example, the effectiveness of immunogenic peptides, pMHCs, engineered cells, binding proteins, and related compositons described herein to increase an immune response (e.g., T cell immune response) against cells of interest, such as hyperproliferative cells, expressing MAGEA1, may be monitored in clinical trials of subjects afflicted with a disorder characterized by MAGEA1 expression. In such clinical trials, the presence of binding and/or T cell activation and/or effector function (e.g., T cell proliferation, killing, and/or cytokine release), may be used as a “read out” or marker of the phenotype of a particular cell, tissue, or system. Similarly, the effectiveness of an adaptive T cell therapy with T cells engineered to express a binding protein (e.g., a TCR, an antigen-binding fragment of a TCR, a CAR, or a fusion protein comprising a TCR and an effector domain) as described herein to increase immune response to cells of interest, such as hyperproliferative cells, that are expressing MAGEA1, may be monitored in clinical trials of subjects having a disorder characterized by MAGEA1 expression. In such clinical trials, the presence of binding and/or T cell activation and/or effector function (e.g., T cell proliferation, killing, or cytokine release), may be used as a “read out” or marker of the phenotype of a particular cell, tissue, or system.

In some embodiments, the present invention provides a method for monitoring the effectiveness of treatment of a therapy (e.g., compounds, drugs, vaccines, cell therapies, and the like) including the steps of a) determining the absence, presence, or level of reactivity between a sample obtained from the subject and one or more binding proteins or related composition, in a first sample obtained from the subject prior to providing at least a portion of the therapy for the disorder characterized by MAGEA1 expression to the subject, and b) determining the absence, presence, or level of reactivity between the one or more binding proteins or related composition, and a sample obtained from the subject present in a second sample obtained from the subject following provision of the portion of the therapy, wherein the presence or a higher level of reactivity in the first sample, relative to the second sample, is an indication that the therapy is efficacious for treating the disorder characterized by MAGEA1 expression in the subject and wherein the absence or a lower level of reactivity in the first sample, relative to the second sample, is an indication that the therapy is not efficacious for treating the disorder characterized by MAGEA1 expression in the subject.

In some embodiments, the present invention provides a method for monitoring the effectiveness of treatment of a subject with an agent (e.g., antibodies, an agonist, antagonist, peptidomimetic, polypeptide, peptide, nucleic acid, small molecule, or other drug candidate identified by the screening assays described herein) including the steps of (i) obtaining a preadministration sample from a subject prior to administration of the agent; (ii) detecting MAGEA1 expression in the preadministration sample; (iii) obtaining one or more post-administration samples from the subject; (iv) detecting MAGEA1 expression in the post-administration samples; (v) comparing the MAGEA1 expresion in the pre-administration sample with the MAGEA1 expression in the post-administration sample; and (vi) altering the administration of the agent to the subject accordingly. Biomarker polypeptide analysis, such as by immunohistochemistry (IHC), may also be used to select patients who will receive therapy, such as immunotherapy.

In addition, the prognostic methods described herein may be used to determine whether a subject may be administered a therapeutic agent to treat a disorder associated with MAGEA1 expression.

f. Clinical Efficacy

Clinical efficacy may be measured by any method known in the art. For example, the response to a therapy relates to any response of the disorder associated with MAGEA1 expression, e.g., a tumor, to the therapy, preferably to a change in the number of cancer cells, tumor mass, and/or tumor volume, such as after initiation of neoadjuvant or adjuvant chemotherapy. Tumor response may be assessed in a neoadjuvant or adjuvant situation where the size of a tumor after systemic intervention may be compared to the initial size and dimensions as measured by CT, PET, mammogram, ultrasound or palpation and the cellularity of a tumor may be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion such as percentage change in tumor volume or cellularity or by using a semi-quantitative scoring system such as residual cancer burden (Symmans et al. (2007) J. Clin. Oncol. 25:4414-4422) or Miller-Payne score (Ogston et al. (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy (e.g., after a few hours, days, weeks or preferably after a few months). A typical endpoint for response assessment is upon termination of neoadjuvant chemotherapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular modulator of biomarkers listed in Table 1 therapeutic regimen is at least 25%, 30%, 35%, 40%, 45%, 50%, 55%, 60%, 65%, 70%, 75%, 80%, 85%, or more.

Additional criteria for evaluating the response to cancer therapy are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence, or metastasis). In addition, criteria for efficacy of treatment may be expanded to include response to chemotherapy, probability of survival, probability of metastasis within a given time period, and probability of tumor recurrence.

For example, in order to determine appropriate threshold values, a particular agent of interest may be administered to a population of subjects and the outcome may be correlated to biomarker measurements that were determined prior to administration of any therapy. The outcome measurement may be pathologic response to therapy given in the neoadjuvant setting. Alternatively, outcome measures, such as overall survival and disease-free survival may be monitored over a period of time for subjects following therapy for whom MAGEA1 expression values are known. In certain embodiments, the same doses of the agent are administered to each subject. The period of time for which subjects are monitored may vary. For example, subjects may be monitored for at least 1, 2, 3, 4, 5, 6, 7, 8, 9, 10, 11, 12, 14, 16, 18, 20, 25, 30, 35, 40, 45, 50, 55, 60 months, or longer. MAGEA1 measurement threshold values that correlate to outcome of a therapy may be determined using well-known methods, such as those described in the Examples section.

### X. Cell Therapy

In another aspect encompassed by the present invention, the methods include adoptive cell therapy, whereby genetically engineered cells expressing the provided molecules targeting an MHC-restricted epitope (e.g., cells expressing a binding protein (e.g., a TCR or CAR) or antigen-binding fragment thereof) are administered to subjects. Such administration may promote activation of immune cells (e.g., T cell activation) in an antigen-targeted manner, such that the cells of interest, such as hyperproliferative cells, that express a MAGEA1 antigen are targeted for destruction.

Thus, the provided methods and uses include methods and uses for adoptive cell therapy. In some embodiments, the methods include administration of the cells or a composition containing the cells to a subject, tissue, or cell, such as one having, at risk for, or suspected of having the disease, condition or disorder. In some embodiments, the cells, populations, and compositions are administered to a subject having the particular disease or condition to be treated (e.g., via adoptive cell therapy, such as by adoptive T cell therapy). In some embodiments, the cells or compositions are administered to the subject, such as a subject having or at risk for the disease or condition. In some embodiments, the methods thereby treat, e.g., ameliorate one or more symptom of the disease or condition.

Methods for administration of cells for adoptive cell therapy are known and may be used in connection with the provided methods and compositions (e.g., U.S. Pat. Publ. No. 2003/0170238, U.S. Pat. No. 4,690,915, Rosenberg (2011) Nat. Rev. Clin. Oncol. 8:577-585, Themeli et al. (2013) Nat. Biotechnol. 31:928-933, Tsukahara et al. (2013) Biochem. Biophys. Res. Commun. 438:84-89, and Davila et al. (2013) PLoS ONE 8:e61338).

In some embodiments, cell therapy (e.g., adoptive cell therapy, such as adoptive T cell therapy) may be carried out by autologous transfer, in which the cells are isolated and/or otherwise prepared from the subject who is to receive the cell therapy, or from a sample derived from such a subject. Thus, in some embodiments, the cells are derived from a subject (e.g., patient) in need of a treatment and the cells, following isolation and processing are administered to the same subject.

In some embodiments, the cell therapy (e.g., adoptive cell therapy, such as adoptive T cell therapy) may be carried out by allogeneic transfer, in which the cells are isolated and/or otherwise prepared from a subject other than a subject who is to receive or who ultimately receives the cell therapy (e.g., a first subject). In such embodiments, the cells then are administered to a different subject (e.g., a second subject) of the same species. In some embodiments, the first and second subjects are genetically identical (syngeneic). In some embodiments, the first and second subjects are genetically similar. In some embodiments, the second subject expresses the same HLA class or supertype as the first subject.

In some embodiments, the subject, to whom the cells, cell populations, or compositions are administered is a primate, such as a human. In some embodiments, the primate is a monkey or an ape. The subject may be male or female and may be any suitable age, including infant, juvenile, adolescent, adult, and geriatric subjects. In some embodiments, the subject is a non-primate mammal, such as a rodent. In some examples, the patient or subject is a validated animal model for disease, adoptive cell therapy, and/or for assessing toxic outcomes such as cytokine release syndrome (CRS).

The binding molecules, such as TCRs, antigen-binding fragments of TCRs (e.g., scTCRs) and chimeric receptors (e.g., CARs) containing the TCR, and cells expressing the same, may be administered by any suitable means, for example, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. Dosing and administration may depend in part on whether the administration is brief or chronic. Various dosing schedules include but are not limited to single or multiple administrations over various time-points, bolus administration, and pulse infusion.

For the prevention or treatment of disease, the appropriate dosage of the binding molecule or cell may depend on the type of disease to be treated, the type of binding molecule, the severity and course of the disease, whether the binding molecule is administered for preventive or therapeutic purposes, previous therapy, the patient's clinical history and response to the binding molecule, and the discretion of the attending physician. The compositions and molecules and cells are in some embodiments suitably administered to the patient at one time or over a series of treatments.

In some embodiments, cells may be administered at 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 5.0×10⁶, 1.0×10⁷, 5.0×10⁷, 1.0×10⁸, 5.0×10⁸, or more, or any range in between or any value in between, cells per kilogram of subject body weight. The number of cells transplanted may be adjusted based on the desired level of engraftment in a given amount of time. Generally, 1×10⁵ to about 1×10⁹ cells/kg of body weight, from about 1×10⁶ to about 1×10⁸ cells/kg of body weight, or about 1×10⁷ cells/kg of body weight, or more cells, as necessary, may be transplanted. In some embodiment, transplantation of at least about 0.1×10⁶, 0.5×10⁶, 1.0×10⁶, 2.0×10⁶, 3.0×10⁶, 4.0×10⁶, or 5.0×10⁶ total cells relative to an average size mouse is effective. For example, in some embodiments, cells, or individual populations of sub-types of cells, may be administered to the subject at a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight. Dosages may vary depending on attributes particular to the disease or disorder and/or patient and/or other treatments.

Engraftment of transplanted cells may be assessed by any of various methods, such as, but not limited to, tumor volume, cytokine levels, time of administration, flow cytometric analysis of cells of interest obtained from the subject at one or more time points following transplantation, and the like. For example, a time-based analysis of waiting 1, 2, 3, 4, 5, 6, 7, 8, 9, 10, 11, 12, 13, 14, 15, 16, 17, 18, 19, 20, 21, 22, 23, 24, 25, 26, 27, 28 days or may signal the time for tumor harvesting (e.g., providing a dose of TCR-T cells infused 28 days apart). Any such metrics are variables that may be adjusted according to well-known parameters in order to determine the effect of the variable on a response to anti-cancer immunotherapy. In addition, the transplanted cells may be co-transplanted with other agents, such as cytokines, extracellular matrices, cell culture supports, and the like.

Cells may also be administered before, concurrently with, or after, other anti-cancer agents.

Two or more cell types may be combined and administered, such as cell-based therapy and adoptive cell transfer of stem cells, cancer vaccines and cell-based therapy, and the like. For example, adoptive cell-based immunotherapies may be combined with the cell-based therapies encompassed by the present invention. In some embodiments, the cell-based agents may be used alone or in combination with additional cell-based agents, such as immunotherapies like adoptive T cell therapy (ACT). For example, T cells genetically engineered to recognize CD19 used to treat follicular B cell lymphoma. Immune cells for ACT may be dendritic cells, T cells such as CD8+ T cells and CD4+ T cells, natural killer (NK) cells, NK T cells, cytotoxic T lymphocytes (CTLs), tumor infiltrating lymphocytes (TILs), lymphokine activated killer (LAK) cells, memory T cells, regulatory T cells (Tregs), helper T cells, cytokine-induced killer (CIK) cells, and any combination thereof. Well-known adoptive cell-based immunotherapeutic modalities, including, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (AIET), cancer vaccines, and/or antigen presenting cells. Such cell-based immunotherapies may be further modified to express one or more gene products to further modulate immune responses, such as expressing cytokines like GM-CSF, and/or to express tumor-associated antigen (TAA) antigens, such as Mage-1, gp-100, and the like. The ratio of an agent encompassed by the present invention, such as cancer cells, to another agent encompassed by the present invention or other composition may be 1:1 relative to each other (e.g., equal amounts of 2 agents, 3 agents, 4 agents, etc.), but may modulated in any amount desired (e.g., 1:1, 1.1:1, 1.2:1, 1.3:1, 1.4:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, or greater).

In some embodiments, for example, where the subject is a human, the dose includes fewer than about 1×10⁸ total binding protein (e.g., TCR- or CAR-expressing cells, T cells, or peripheral blood mononuclear cells (PBMCs), e.g., in the range of about 1×10⁶ to 1×10⁸ such cells, such as 2×10⁶, 5×10⁶, 1×10⁷, 5×10⁷, or 1×10⁸ or total such cells, or the range between any two of the foregoing values.

In some embodiments, the cells or related compositions described herein, such as nucleic acids, host cells, pharmaceutical formulations, and the like, may be administered as part of a combination treatment, such as simultaneously with or sequentially with, in any order, another therapeutic intervention, such as another antibody or engineered cell or receptor or agent, such as a cytotoxic or therapeutic agent.

In some embodiments, the cells or related composition may be co-administered with one or more additional therapeutic agents or in connection with another therapeutic intervention, either simultaneously or sequentially in any order. In some contexts, the cells or related composition are co-administered with another therapy sufficiently close in time such that the cell populations enhance the effect of one or more additional therapeutic agents, or vice versa. In some embodiments, the cells or related composition are administered prior to the one or more additional therapeutic agents. In some embodiments, the cells or related composition are administered after to the one or more additional therapeutic agents.

In some embodiments, the biological activity of the cells or related composition is measured by any of a number of known methods once the cells or related composition are administered to a subject (e.g., a human). Parameters to assess include specific binding of an engineered or natural T cell or other immune cell to antigen, in vivo, e.g., by imaging, or in vitrolex vivo, e.g., by ELISA or flow cytometry. In some embodiments, the ability of the cells to destroy target cells (cytotoxicity) may be measured using any suitable assay or method known in the art (e.g., Kochenderfer et al. (2009) J. Immunother. 32:689-702 and

Herman et al. (2004) J. Immunol. Meth. 285:25-40). In some embodiments, the biological activity of the cells also may be measured by assaying expression and/or secretion of certain cytokines, such as CD107a, IFNγ, IL-2, and TNF alpha. In some embodiments, the biological activity is measured by assessing clinical outcome, such as reduction in viral burden or load.

In some embodiments, cells are modified in any number of ways, such that their therapeutic or prophylactic efficacy is increased. For example, the binding protein (e.g., engineered TCR, CAR, or antigen-binding fragment thereof) expressed by the population may be conjugated either directly or indirectly through a linker to a targeting moiety. The practice of conjugating compounds to targeting moieties is well-known in the art (e.g., Wadwa et al. (1995) J. Drug Targeting 3:111 and U.S. Pat. No. 5,087,616).

Immune cells, such as cytotoxic lymphocytes, may be obtained from any suitable source such as peripheral blood, spleen, and lymph nodes. The immune cells may be used as crude preparations or as partially purified or substantially purified preparations, which may be obtained by standard techniques, including, but not limited to, methods involving immunomagnetic or flow cytometry techniques using antibodies.

In certain aspects, the MAGEA1 immunogenic peptides described herein, or a nucleic acid encoding such MAGEA1 immunogenic peptides, may be used in compositions and methods for providing MAGEA1-primed, antigen-presenting cells, and/or MAGEA1-specific lymphocytes generated with these antigen-presenting cells. In some embodiments, such antigen-presenting cells and/or lymphocytes are used in the treatment and/or prevention of a disorder associated with MAGEA1 expression.

In some aspects, provided herein are methods for making MAGEA1-primed, antigen-presenting cells by contacting antigen-presenting cells with a MAGEA1 immunogenic peptide described herein, or nucleic acids encoding the at least one MAGEA1 immunogenic peptide, alone or in combination with an adjuvant, in vitro under a condition sufficient for the at least one MAGEA1 immunogenic polypeptide to be presented by the antigen-presenting cells.

In some embodiments, MAGEA1 immunogenic polypeptide, or nucleic acid encoding the MAGEA1 immunogenic polypeptide, alone or in combination with an adjuvant, may be contacted with a homogenous, substantially homogenous, or heterogeneous composition comprising antigen-presenting cells. For example, the composition may include but is not limited to whole blood, fresh blood, or fractions thereof such as, but not limited to, peripheral blood mononuclear cells, buffy coat fractions of whole blood, packed red cells, irradiated blood, dendritic cells, monocytes, macrophages, neutrophils, lymphocytes, natural killer cells, and natural killer T cells. If, optionally, precursors of antigen-presenting cells are used, the precursors may be cultured under suitable culture conditions sufficient to differentiate the precursors into antigen-presenting cells. In some embodiments, the antigen-presenting cells (or precursors thereof) are selected from monocytes, macrophages, cells of myeloid lineage, B cells, dendritic cells, or Langerhans cells.

The amount of the MAGEA1 immunogenic polypeptide, or nucleic acid encoding the MAGEA1 immunogenic polypeptide, alone or in combination with an adjuvant, to be placed in contact with antigen-presenting cells may be determined by one of ordinary skill in the art by routine experimentation. Generally, antigen-presenting cells are contacted with the MAGEA1 immunogenic polypeptide, or nucleic acid encoding the MAGEA1 immunogenic polypeptide, alone or in combination with an adjuvant, for a period of time sufficient for cells to present the processed forms of the antigens for the modulation of T cells. In one embodiment, antigen-presenting cells are incubated in the presence of the MAGEA1 immunogenic polypeptide, or nucleic acid encoding the MAGEA1 immunogenic polypeptide, alone or in combination with an adjuvant, for less than about a week, illustratively, for about 1 minute to about 48 hours, about 2 minutes to about 36 hours, about 3 minutes to about 24 hours, about 4 minutes to about 12 hours, about 6 minutes to about 8 hours, about 8 minutes to about 6 hours, about 10 minutes to about 5 hours, about 15 minutes to about 4 hours, about 20 minutes to about 3 hours, about 30 minutes to about 2 hours, and about 40 minutes to about 1 hour. The time and amount of the MAGEA1 immunogenic polypeptide, or nucleic acid encoding the MAGEA1 immunogenic polypeptide, alone or in combination with an adjuvant, necessary for the antigen presenting cells to process and present the antigens may be determined, for example using pulse-chase methods wherein contact is followed by a washout period and exposure to a read-out system e.g., antigen reactive T cells.

In certain embodiments, any appropriate method for delivery of antigens to the endogenous processing pathway of the antigen-presenting cells may be used. Such methods include but are not limited to, methods involving pH-sensitive liposomes, coupling of antigens to adjuvants, apoptotic cell delivery, pulsing cells onto dendritic cells, delivering recombinant chimeric virus-like particles (VLPs) comprising antigen to the MHC class I processing pathway of a dendritic cell line.

In one embodiment, solubilized MAGEA1 immunogenic polypeptide is incubated with antigen-presenting cells. In some embodiments, the MAGEA1 immunogenic polypeptide may be coupled to a cytolysin to enhance the transfer of the antigens into the cytosol of an antigen-presenting cell for delivery to the MHC class I pathway. Exemplary cytolysins include saponin compounds such as saponin-containing Immune Stimulating Complexes (ISCOM5), pore-forming toxins (e.g., an alpha-toxin), and natural cytolysins of gram-positive bacteria such as listeriolysin O (LLO), streptolysin O (SLO), and perfringolysin O (PFO).

In some embodiments, antigen-presenting cells, such as dendritic cells and macrophage, may be isolated according to methods known in the art and transfected with polynucleotides by methods known in the art for introducing a nucleic acid encoding the MAGEA1 immunogenic polypeptide into the antigen-presenting cell. Transfection reagents and methods are known in the art and commercially available. For example, RNA encoding MAGEA1 immunogenic polypeptide may be provided in a suitable medium and combined with a lipid (e.g., a cationic lipid) prior to contact with antigen-presenting cells. Non-limiting examples of such lipids include LIPOFECTIN™ and LIPOFECTAMINE™. The resulting polynucleotide-lipid complex may then be contacted with antigen-presenting cells. Alternatively, the polynucleotide may be introduced into antigen-presenting cells using techniques such as electroporation or calcium phosphate transfection. The polynucleotide-loaded antigen-presenting cells may then be used to stimulate T lymphocyte (e.g., cytotoxic T lymphocyte) proliferation in vitro, ex vivo, or in vivo. In one embodiment, the ex vivo expanded T lymphocyte is administered to a subject in a method of adoptive immunotherapy. In certain aspects, provided herein is a composition comprising antigen-presenting cells that have been contacted in vitro with a MAGEA1 immunogenic polypeptide, or a nucleic acid encoding a MAGEA1 immunogenic polypeptide, alone or in combination with an adjuvant under a condition sufficient for a MAGEA1 immunogenic epitope to be presented by the antigen-presenting cells.

In some aspects, provided herein is a method for preparing lymphocytes specific for a MAGEA1 protein. The method comprises contacting lymphocytes with the antigen-presenting cells described above under conditions sufficient to produce a MAGEA1 protein-specific lymphocyte capable of eliciting an immune response against a cell that is infected by the MAGEA1 virus. Thus, the antigen-presenting cells also may be used to provide lymphocytes, including T lymphocytes and B lymphocytes, for eliciting an immune response against cell that is infected by the MAGEA1 virus.

In some embodiments, a preparation of T lymphocytes is contacted with the antigen-presenting cells described above for a period of time, (e.g., at least about 24 hours) to priming the T lymphocytes to a MAGEA1 immunogenic epitope presented by the antigen-presenting cells.

In some embodiments, a population of antigen-presenting cells may be co-cultured with a heterogeneous population of peripheral blood T lymphocytes together with a MAGEA1 immunogenic polypeptide, or a nucleic acid encoding a MAGEA1 immunogenic polypeptide, alone or in combination with an adjuvant. The cells may be co-cultured for a period of time and under conditions sufficient for MAGEA1 epitopes included in the MAGEA1 polypeptides to be presented by the antigen-presenting cells and the antigen-presenting cells to prime a population of T lymphocytes to respond to cells is infected by the MAGEA1 virus. In certain embodiments, provided herein are T lymphocytes and B lymphocytes that are primed to respond to cells that is infected by the MAGEA1 virus.

T lymphocytes may be obtained from any suitable source such as peripheral blood, spleen, and lymph nodes. The T lymphocytes may be used as crude preparations or as partially purified or substantially purified preparations, which may be obtained by standard techniques including, but not limited to, methods involving immunomagnetic or flow cytometry techniques using antibodies.

In certain aspects, provided herein is a composition (e.g., a pharmaceutical composition) comprising the antigen-presenting cells or the lymphocytes described above, and a pharmaceutically acceptable carrier and/or diluent. In some embodiments, the composition further comprises an adjuvant as described above.

In certain aspects and as further described above, provided herein is a method for eliciting an immune response to the cell is infected by the MAGEA1 virus, the method comprising administering to the subject the antigen-presenting cells or the lymphocytes described above in effective amounts sufficient to elicit the immune response. In some embodiments, provided herein is a method for treatment or prophylaxis of a disorder characterized by MAGEA1 expression, the method comprising administering to the subject an effective amount of the antigen-presenting cells or the lymphocytes described above. In one embodiment, the antigen-presenting cells or the lymphocytes are administered systemically, preferably by injection. Alternately, one may administer locally rather than systemically, for example, via injection directly into tissue, preferably in a depot or sustained release formulation.

In certain embodiments, the antigen-primed antigen-presenting cells described herein and the antigen-specific T lymphocytes generated with these antigen-presenting cells may be used as active compounds in immunomodulating compositions for prophylactic or therapeutic treatment of a disorder characterized by MAGEA1 expression. In some embodiments, the MAGEA1-primed antigen-presenting cells described herein may be used for generating CD8⁺T lymphocytes, CD4⁺ T lymphocytes, and/or B lymphocytes for adoptive transfer to the subject. Thus, for example, MAGEA1-specific lymphocyte may be adoptively transferred for therapeutic purposes in subjects afflicted with a disorder characterized by MAGEA1 expression.

In certain embodiments, the antigen-presenting cells and/or lymphocytes described herein may be administered to a subject, either by themselves or in combination, for eliciting an immune response, particularly for eliciting an immune response to cells expressing MAGEA1. In some embodiments, the antigen-presenting cells and/or lymphocytes may be derived from the subject (i.e., autologous cells) or from a different subject that is MHC matched or mismatched with the subject (e.g., allogeneic).

Single or multiple administrations of the antigen-presenting cells and lymphocytes may be carried out with cell numbers and treatment being selected by the care provider (e.g., physician). In some embodiments, the antigen-presenting cells and/or lymphocytes are administered in a pharmaceutically acceptable carrier. Suitable carriers may be growth medium in which the cells were grown, or any suitable buffering medium such as phosphate buffered saline. The cells may be administered alone or as an adjunct therapy in conjunction with other therapeutics.

In another aspect encompassed by the present invention, provided herein is a method for eliciting an immune response to a cell that expresses MAGEA1, the method comprising administering to the subject cells described herein expressing a binding protein (e.g., engineered TCR, CAR, or antigen-binding fragment thereof) in effective amounts sufficient to elicit the immune response. In some embodiments, provided herein is a method for treatment or prophylaxis of a disorder characterized by MAGEA1 expression (e.g., a non-malignant disorder, a hyperproliferative disorder, or a relapse of a hyperproliferative disorder characterized by MAGEA1 expression), the method comprising administering to the subject an effective amount of the cells described herein expressing a binding protein (e.g., engineered TCR, CAR, or antigen-binding fragment thereof). In one embodiment, the cells are administered systemically, such as by injection. Alternately, one may administer locally rather than systemically, for example, via injection directly into tissue, such as in a depot or sustained release formulation.

In some embodiments, the cells described herein expressing a binding protein (e.g., engineered TCR, CAR, or antigen-binding fragment thereof) may be used as active compounds in immunomodulating compositions for prophylactic or therapeutic treatment of a disorder characterized by MAGEA1 expression (e.g., a non-malignant disorder, a hyperproliferative disorder, or a relapse of a hyperproliferative disorder characterized by MAGEA1 expression). In some embodiments, MAGEA1-primed antigen-presenting cells may be used for generating lymphocytes (e.g., CD8⁺ T lymphocytes, CD4⁺ T lymphocytes, and/or B lymphocytes), for further use in adoptive transfer to the subject with the cells described herein expressing a binding protein (e.g., engineered TCR, CAR, or antigen-binding fragment thereof).

In some embodiments, the cells described herein expressing a binding protein (e.g., engineered TCR, CAR, or antigen-binding fragment thereof), either alone or in combination with the lymphocytes, may be administered to a subject for eliciting an immune response, particularly for eliciting an immune response to cells are expressing MAGEA1.

As described above, single or multiple administrations of the cells described herein expressing a binding protein (e.g., engineered TCR, CAR, or antigen-binding fragment thereof) cells, either alone or in combination with the lymphocytes, may be carried out with cell numbers and treatment being selected by the care provider (e.g., physician). Similarly, the cells, either alone or in combination with lymphocytes, may be administered in a pharmaceutically acceptable carrier. Suitable carriers may be growth medium in which the cells were grown, or any suitable buffering medium such as phosphate buffered saline. Cells may be administered alone or as an adjunct therapy in conjunction with other therapeutics.

### XI. Kits and Devices

The present invention also encompasses kits and devices. For example, the kit or devie may comprise binding proteins, nucleic acids or vectors comprising sequences encoding binding proteins, host cells comprising nucleic acids or vectors and/or expressing the binding proteins as described herein, stable MHC-peptide complexes, adjuvants, detection reagents, and combinations thereof, packaged in a suitable container and may further comprise instructions for using such reagents. The kit may also contain other components, such as administration tools packaged in a separate container. The kit may be promoted, distributed, or sold as a unit for performing the methods encompassed by the present invention.

The disclosure is further illustrated by the following examples, which should not be construed as limiting.

### EXAMPLES

### Example 1: Materials and Methods for Example 3

a. Human Peripheral Blood Mononuclear Cell Collection

HLA-A*02:01-positive healthy donor leukopaks were collected by HemaCare (Los Angeles, CA), StemExpress (Placerville, CA), and Discovery Life Sciences (Huntsville, AL) using their IRB-approved protocols. Peripheral blood mononuclear cells (PBMCs) were isolated from fresh leukopaks from HemaCare and Discovery Life Sciences by density gradient centrifugation using Lymphocyte Separation Medium (Corning, Corning, NY). PBMCs contained in the lymphocyte layer were collected following centrifugation, washed 3 times with DPBS (Cytiva, Marlborough, MA), and counted. PBMCs were isolated from StemExpress leukopaks either by density gradient centrifugation as above, or using a Custom Leukopak PBMC Isolation kit (Miltenyi Biotec, Auburn, CA) on the MultiMACS Cell24 Separator Plus instrument, version 3 (Miltenyi Biotec) per the manufacturer's instructions. Isolated PBMCs were frozen in CryoStor CS10 (StemCell Technologies, Cambridge, MA) and stored in liquid nitrogen.

b. TCRs Screens
i. DC Culture

Monocyte isolation was performed on day-4 using PBMCs isolated from HLA-A*02:01-positive healthy donors with the EasySep Human CD14 Positive Selection Kit II (StemCell Technologies) according to the manufacturer's instructions. Purity and costimulatory molecule expression were assessed using fluorescently-labeled antibodies specific for CD14 (M5E2, BioLegend, Dedham, MA), HLA-A2 (BB7.2, BioLegend), CD80 (2D10, BioLegend), CD83 (HB15e, BioLegend), and CD86 (IT2.2, BioLegend); CD14 expression was >90%. CD14⁺ monocytes were resuspended in AIM-V media (Thermo Fisher Scientific) supplemented with recombinant human GM-CSF and IL-4 (R&D Systems, Minneapolis, MN) at final concentrations of 800 IU/mL and 1000 IU/mL, respectively. On day-2, recombinant human TNF-α (10 ng/ml), IL-6 (1000 IU/mL), and IL-1β (2 ng/ml) (R&D Systems) as well as PGE2 (1 μg/mL, StemCell Technologies) were added to cultured monocytes.

ii. CD8 Naive T Cell Isolation

On day-1, autologous CD8 naive T cells were isolated from PBMCs from HLA-A*02:01-expressing healthy donors using the EasySep™ Human Naïve CD8+ T Cell Isolation Kit II (StemCell Technologies) according to the manufacturer's instructions. Purity was assessed using fluorescently-labeled antibodies specific for CD8a (HIT8a, BioLegend), CD45RO (UCHL1, BioLegend), CD45RA (HI100, BioLegend), CD56 (5.1H11, BioLegend), CD57 (HCD57, BioLegend), and CCR7 (G043H7, BioLegend); purity of naive CD8α⁺ T cells was >90%. Cells were rested overnight at 37° C., 5% CO₂ in T cell medium (X-VIVO 15 serum-free medium [Lonza, Rockland, MD] containing 10% human serum [Sigma Aldrich, St. Louis, MO], 1% penicillin-streptomycin [Thermo Fisher Scientific], 1% GlutaMAX [Thermo Fisher]), supplemented with 10 ng/ml recombinant human IL-7 (R&D Systems).

iii. Co-Culture

On day 0, CD8 T cell purity was reassessed using the identical antibody panel as on day 3, and DC maturation was confirmed by upregulation of HLA-A2, CD80, CD83, and CD86 and downregulation of CD14. DCs were pulsed with 1 μM MAGE-A1₂₇₈₋₂₈₆ peptide (KVLEYVIKV (SEQ ID NO: 1), GenScript [Piscataway, NJ]) for 3 hours at 37° C., 5% CO₂. Pulsed DCs were co-cultured with rested CD8 naïve T cells in T cell medium supplemented with recombinant human IL-12 (10 ng/ml) and IL-21 (60 ng/ml) (R&D Systems). Co-cultures were supplemented with recombinant human IL-7 and IL-15 (R&D Systems) between days 3 and 10. Dextramer staining for MAGE-A1-specific cells was performed on day 11, using A*02:01 MAGE-A1₂₇₈₋₂₈₆ (KVLEYVIKV (SEQ ID NO: 1)) dextramer (Immudex, Copenhagen, Denmark), CD8α and TCRα/β (IP26, BioLegend), and DAPI (Thermo Fisher Scientific) according to the manufacturer's instructions.

iv. Antigen-Specific Cell Sorting

On day 12, cells were collected and stained with A*02:01 MAGE-A1₂₇₈₋₂₈₆ (KVLEYVIKV (SEQ ID NO: 1)) dextramer, as well as with antibodies specific for CD8a and TCRα/β, and with DAPI as on day 11, and MAGE-A1₂₇₈₋₂₈₆-positive cells (CD8α⁺, DAPI⁻, TCRα/β⁺, MAGE-A1⁺) were sorted using a Sony SH800S cell sorter (Sony Biotechnology, San Jose, CA), BigFoot Cell Sorter (ThermoFisher Scientific), or MoFlo Astrios Cell Sorter (Beckman Coulter, Brea, CA). Sorted cells were subjected to single cell TCRα/β sequencing using the 10× Genomics platform (Pleasanton, CA).

v. Flow Cytometry

Cells were acquired using a CytoFLEX flow cytometer (Beckman Coulter, Indianapolis, IN) and analyzed using FlowJo software (version 10, TreeStar, Ashland, OR).

c. Single Cell TCRα/β Sequencing Using the 10× Genomics Platform

Single-cell TCR-seq (scTCR-seq) libraries were prepared according to the 10× Genomics Single Cell V (D) J Reagent Kit (v1) protocol (10× Genomics). A target number of 10,000 cells per sample was captured in droplets (GEMs) within the 10× Genomics Chromium instrument before undergoing reverse transcription. Following reverse transcription, GEMs were broken, and barcoded cDNA was purified from the samples using Silane magnetic beads. cDNA was amplified as follows: 98° C. for 45 seconds; 13 cycles of 98° C. for 20 seconds, 67° C. for 30 seconds, 72° C. for 1 minute; 72° C. for 1 minute). Following sample purification with 0.6× SPRIselect beads (Beckman Coulter), 2 μL of each library was used for TCR sequence enrichment. TCR sequence enrichment consisted of 2 rounds of PCR to amplify both the TCRα and TCRβ chain transcripts. TCR-enriched libraries were subsequently fragmented, end-repaired, and amplified with indexing primers. The fully-assembled libraries were sequenced on an Illumina NextSeq instrument (Illumina, San Diego, CA) using a High Output v2.5 kit (150 cycles) with read lengths: 26 bp (read 1), 8 bp (i7 index), and 98 bp (read 2). Sequenced scTCRseq reads were processed using the cellranger 3.1.0 pipeline. Reads were aligned to the GRCh38 reference genome, and the cellranger vdj module was used to annotate TCR consensus sequences.

d. Materials

TABLE 6

| | |
|---|---|
| Media | X-VIVO 15, serum-free hematopoietic cell |
| | medium, with L-Glutamine, gentamycin and |
| | phenol red: Lonza, 04-418Q |
| | LymphoONE ™ T-Cell Expansion Xeno-Free |
| | Medium: Takara, WK552 |
| | Human serum (heat-inactivated): Sigma-Aldrich, |
| | H3667-100ML |
| | AIMV medium: [+] L-glutamine, |
| | [+] 50 μg/mL streptomycin sulfate, |
| | [+] 10 μg/mL gentamicin sulfate: Gibco, 12055-083 |
| | Penicillin streptomycin: Gibco, 15149-122 |
| | GlutaMAX ™ supplement: Gibco, 35050061 |
| | 30% BSA in PBS: Sigma Aldrich, A9576-50ML |
| T cell | Recombinant human IL-7: R&D Systems, 207-IL-025 |
| cytokines: | Recombinant human IL-12: R&D Systems, 219-IL-005 |
| | Recombinant human IL-15: R&D Systems, 247-ILB-005 |
| DC | Recombinant human GM-CSF: R&D |
| cytokines: | Systems, 215-GM-050 |
| | Recombinant human IL-4: R&D Systems, 204-IL-050 |
| | Recombinant human TNF-α: R&D Systems, 210-TA-050 |
| | Recombinant human IL-1β/IL-1F2: R&D |
| | Systems, 201-LB-025 |
| | Recombinant human IL-6: R&D Systems, 206-IL-050 |
| Kits | EasySep ™ Human Naive CD8+ T Cell Isolation |
| | Kit II: STEMCELL Technologies, 17968 |
| | EasySep ™ Human CD14 Positive Selection |
| | Kit II: STEMCELL Technologies, 17858 |
| Antibodies | Anti-human CD8α PerCp-Cy5.5 (clone: HIT8a): |
| (all | BioLegend, 300924 |
| antibodies | Anti-human CD56 PE (clone: 5.1H11): |
| are from | BioLegend, 362508 |
| BioLegend): | Anti-human CD57 PE (clone: HCD57): |
| | BioLegend, 322312 |
| | Anti-human CD45RO FITC (clone: UCHL1): |
| | BioLegend, 304204 |
| | Anti-human CD45 RA APC (clone: HI100): |
| | BioLegend, 304112 |
| | Anti-human CCR7 BV605 (clone: G043H7): |
| | BioLegend, 353224 |
| | Anti-Human CD14 Alexa Fluor 488 ® |
| | (clone: M5E2): BioLegend, 301811 |
| | Anti-human CD83 PE-Cy7 (clone: HB15e): |
| | BioLegend, 305326 |
| | Anti-human HLA-A2 PE (clone: BB7.2): |
| | BioLegend, 343306 |
| | Anti-human CD80 APC (clone: 2D10): |
| | BioLegend, 305220 |
| | Anti-human CD86 Brilliant Violet 605 ™ |
| | (clone IT2.2): BioLegend, 305430 |
| | Anti-human TCRα/β FITC (clone: IP26): |
| | BioLegend, 221058 |
| | DAPI: Thermo Scientific: Thermo Scientific, 62248 |
| | Cytofix: BD Biosciences, 554655 |
| Peptides: | MAGE-A1₂₇₈₋₂₈₆ (KVLEYVIKV |
| Dextramers: | (SEQ ID NO: 1)): GenScript |
| | A*02:01 MAGE-_{A1278-286} dextramer: Immudex, WB0347 |
| Reagent | T cell medium: X-VIVO or LymphoONE |
| preparation: | with 10% human serum, |
| | 1% penicillin/streptomycin, and 1x GlutaMAX |
| | Dextramer buffer: PBS + 1% BSA |
| 10x | Chromium Single Cell 5′ Library & Gel Bead kit |
| Genomics | v1: 10x Genomics, PN1000006 |
| reagents | Chromium Single Cell V(D)J Enrichment Kit, Human |
| | T Cell: 10x Genomics, PN1000005 |
| | Chromium Chip A Single Cell Kit: |
| | 10x Genomics, PN1000152 |
| | Single Index Kit T Set A: 10x Genomics, PN1000213 |
| | SPRIselect reagent kit: Beckman Coulter, B23318 |
| | High Sensitivity D5000 ScreenTape: Agilent |
| | Technologies, 5067-5592 |
| | High Sensitivity D5000 Reagents: |
| | Agilent Technologies, 5067-5593 |
| | Qubit ™ dsDNA HS Assay Kit: Invitrogen, Q32854 |

### Example 2: Materials and Methods for Examples 4-9

a. Lentiviral Packaging and Quantification of Lentiviral Titer

Lenti-X GoStix Plus (Takara Bio USA, Mountain View, CA) was used to package and quantify MAGEA1₂₇₈₋₂₈₆ viral constructs. Briefly, MAGEA1₂₇₈₋₂₈₆ viral constructs were diluted 1:100 with PBS. 20 ul of MAGEA1₂₇₈₋₂₈₆ viral supernatant was appled to the Lenti-X GoStix Plus cassette sample well and then 80 ul of Chase buffer was applied. A lateral flow test was run for 10 minutes, and a test band (T) started to appear within 5 minutes and reached maximum intensity at 10 minutes if the sample contained sufficient lentivirus. The control band (C) always appeared when the test was functioning properly. After 10 minutes, proper alignment and focal length for imaging was achieved by using the outline of the cassette in the scanning window. The sample name appeared below the outline of the cassette. Once proper alignment was achieved, the outline turned green, and the cassette was automatically scanned.

To calculate the actual IFU/ml for an unknown stock, a reference virus with known titer measured by CD8 expression was used (a virus stock for which the IFU/ml is known) and tested to obtain both an infectious unit value as well as a GoStix Value GV. The IFU/GV ratio was calculated for the reference virus. The unknown sample was analyzed using Lenti-X GoStix Plus to obtain the GV (ng/ml p24) and perform calculations [Formula: GV (unknown)×(IFU/ml)/GV (reference)=IFU/ml (unknown)] to determine your IFU/ml.

b. Functional Evaluation of MAGEA1₂₇₈₋₂₈₆-Specific TCRs
i. Engineering T Cells to Express MAGEA1₂₇₈₋₂₈₆-Specific TCRs

Pan T cells were isolated the StraightFrom® Leukopak® CD3 Microbead Kit (Miltenyi Biotec) according to manufacturer's protocol. Isolated T cells were activated with ImmunoCult CD3/CD28/CD2 T cell activator cocktail (StemCell Technologies) and cultured overnight in complete T cell media (RPMI 1640 supplemented with 10% heat-inactivated fetal bovine serum (FBS), 100 IU/mL penicillin, 100 μg/mL streptomycin, recombinant human IL-2 [50U/mL, PeproTech, Cranbury, NJ], recombinant human IL-15 [5 ng/ml, R&D Systems], and recombinant human IL-7 [5 ng/mL, (R&D Systems]). 24 hours post-activation, T cells were transduced with MAGEA1₂₇₈₋₂₈₆ TCR viral supernatants. 24 hours post-transduction, T cells were transferred either to G-REX® plates (Wilson Wolf) or VECELL® 96-well plates (Cosmo Bio Co.) and expanded for a total of 7-11 days post-activation. T cell cultures were supplemented with fresh IL-2 [50U/mL, PeproTech, Cranbury, NJ], recombinant human IL-15 [5 ng/mL, R&D Systems], and recombinant human IL-7 [5 ng/ml, (R&D Systems] every 2-3 days and/or split to maintain optimal cell densities.

ii. Flow Cytometry of Engineered MAGEA1₂₇₈₋₂₈₆ TCR-Transduced Pan T Cells

Engineered pan T cells were stained with HLA-A*02:01 MAGEA1₂₇₈₋₂₈₆ (KVLEYVIKV (SEQ ID NO: 1)) (Immudex) dextramer, TCR α/β PE-Cy7 (IP26, BioLegend), CD8 PerCP-Cy5.5 (HIT8a, BioLegend), CD4 APC-Cy7 (OKT4, Biolegend), and CD34 Alexa Fluor 488 (QBEND/10, R&D Systems) and DAPI as per the manufacturers' instructions. Cells were then run on the CytoFLEX flow cytometer (Beckman Coulter) and analyzed using FlowJo software (version 10, TreeStar).

iii. Cell Lines

The myeloma cell line U266B1 (ATCC TIB-196), NSCLC cell line NCI-H1703 (ATCC CRL-5889), and malignant melanoma cell lines A-375 (ATCC CRL-1619)/SK-MEL-5 (ATCC HTB-70) and kidney cell line HEK293T (ATCC CRL-3216) were purchased from the American Type Culture Collection (ATCC, Manassas, VA). T2 cells were cultured in IMDM containing 20% heat-inactivated FBS 1% penicillin-streptomycin [Thermo Fisher Scientific]. U266B1 cells were cultured in RPMI 1640 containing 15% heat-inactivated FBS and 1% penicillin-streptomycin [Thermo Fisher Scientific]. NCI-H1703 were maintained in RPMI 1640 containing 10% heat-inactivated FBS and 1% penicillin-streptomycin [Thermo Fisher Scientific]. Hs936.T, A-375, SK-MEL-5 and HEK293T were cultured in DMEM containing 10% heat-inactivated FBS, 1% penicillin-streptomycin [Thermo Fisher Scientific].

iv. Generation of Stable Cell Lines Expressing Incucyte® Nuclight Red

NCI-H1703, Hs936.T, A-375, and A2-HEK293T cells were transduced with Incucyte® NucLight Red Lentivirus Reagent (EF-1α promoter, puromycin selection) (Sartorius) in serum-free media at an MOI of 5. 24 hours post transduction, cells were washed and resuspended in their respective cell line media and cultured at 37° C., 5% CO₂. 3 days post-transduction, puromycin (Gibco, Waltham, MA) was added to the cultures at a pre-determined concentration (ranging from 0.5 μg/mL to 1 ug/mL) to select for transduced cells. Cultures were expanded under puromycin selection until they were at least 98% Nuclight Red-positive as determined by flow cytometric analysis.

### V. In Vitro Cytotoxicity Assay

In vitro cytotoxicity assays for adherent cell lines were performed in 96-well flat-bottom tissue culture plates without coating with poly-L-ornithine. Here the adherent cells were plated and allowed to attach the day before T cells were added. Where indicated, T cells were co-cultured with Nuclight Red-expressing NCI-H1703, Hs936.T, A-375, or A2-HEK293T cells at E:T ratios ranging from 4:1 to 1:4 as indicated. Data were acquired on an Incucyte S3 instrument (Sartorius), and target cell growth was quantified on the Incucyte S3 as a readout of T cell cytotoxicity.

vi. Cytokine Production Assay

T cells were co-cultured with Nuclight Red-expressing expressing NCI-H1703, Hs936.T, A-375, SKMEL5 and A2-HEK293T cells at an E:T of 1:1. Supernatants were harvested 24 hours later and frozen at −80° C. Supernatants were thawed and loaded on an Analyte cartridge (ProteinSimple, San Jose, CA) to evaluate the levels of IFN-γ using the Ella instrument (ProteinSimple).

c. Alloreactivity and Safety Screens
i. T-Cell Engineering

Primary CD3+ T cells were isolated from Leukopaks using the StraightFrom® Leukopak® CD3 Microbead Kit (Miltenyi Biotec) (Alloreactivity), or primary CD8+ T cells were isolated from Leukopaks using the StraightFrom® Leukopak® CD8 Microbead Kit (Miltenyi Biotec) according to manufacturer's protocol. Isolated cells were frozen in CryoStor® CS10 (Stem Cell Technologies) and stored in liquid nitrogen until use. On day −1, CD3⁺ or CD8⁺ T cells were thawed, washed with complete T cell medium (RPMI 1640 supplemented with 10% heat-inactivated fetal bovine serum (FBS), 100 IU/mL penicillin, 100 μg/mL streptomycin, recombinant human IL-2 [50U/mL, PeproTech, Cranbury, NJ], recombinant human IL-15 [5 ng/ml, R&D Systems], and recombinant human IL-7 [5 ng/ml, (R&D Systems]). On day 0, T cells were washed and resuspended in fresh T cell medium and activated using ImmunoCult™ human CD3/CD28/CD2 T cell activator (5 μL/1×10⁶ T cells, Stem Cell Technologies). On day 1, cells were washed and resuspended in fresh complete T cell medium, and plated at 1×10⁶ cells per well. Triplicate wells were transduced with lentiviral particles to express MAGE-A1-1479. On day 2, cells were washed, triplicates were resuspended and combined in fresh complete T cell medium, and expanded until day 5 in 1 well of a G-REX® 6-well plate (Wilson Wolf).

ii. Generation of 96-Well-Based MHC-Expressing Arrays for Alloreactivity Screens

Endogenous HLA-A/B/C were knocked out in HEK293T cells using CRISPR-Cas engineering. Guide RNAs (gRNAs) were designed against sequences conserved acrss the HLA-A, HLA-B, and HLA-C loci using the multicrispr.net tool (Prykhozhij, 2015). The following guides were selected: CRISPR-ALL-1: CGGCTACTACAACCAGAGCG (SEQ ID NO: 39), CRISPR-ALL-2: AGATCACACTGACCTGGCAG (SEQ ID NO: 40), CRISPR-ALL-3: AGGTCAGTGTGATCTCCGCA (SEQ ID NO: 41). gRNAs were cloned into the LentiCRISPR V2 vector using BsmBI sites. HEK293T cells were transfected with plasmid guide constructs using Mirus TransIT (Mirus Bio, Madison, WI). After 7 days, MHC-knockout (MHC-KO) cells were sorted using a pan-MHC antibody (BioLegend). Single-cell clones were expanded, and the absence of MHC was verified by flow cytometry. B2M-knockout (B2M-KO) cells were used as a positive control for the complete absence of surface MHC expression. B2M was knocked out in HEK293T cells by electroporating CRISPR RNPs targeting B2M using the guide RNA: GGCCACGGAGCGAGACATCT (SEQ ID NO: 42).

MHC-null HEK293T cells were transduced with IncuCyte® NucLight™ Red virus (Essen BioScience). Transduced cells were sorted for NucLight™ Red expression using a Sony SH800 sorter. To generate an MHC-expressing array, MHC-null NucLight™ Red-expressing HEK293T cells were transduced with the most common 110 MHCs (pHAGE-EF1a-MHC—UBC-NAT) in individual wells in 96-well plates. Transduced cells were selected with nourseothricin (400 μg/ml) for one week. Cells expressing the most common 110 MHCs were passaged and stored in 96-well plates as an array. Expression of individual MHC alleles was verified by staining using a pan-MHC antibody (BioLegend).

To generate the positive control for the assay, MHC-null HEK293T cells were transduced with IncuCyte® NucLight™ Red virus (Essen BioScience) and sorted for NucLight™ Red expression using a Sony SH800 sorter. HLA-A*02:01 (pHAGE-EFla-MHC—UBC-NAT) was then introduced into the cells using lentiviral transduction. Transduced cells were selected with nourseothricin (400 μg/ml) for one week. These cells were then transduced with an ORF construct (pHAGE-CMV-MAGEA1-EFS-AmCyan) expressing the MAGE-A1 ORF which contains the MAGEA1₂₇₈₋₂₈₆ epitope (KVLEYVIKV (SEQ ID NO: 1)). Transduced cells were then sorted for AmCyan expression using a Bigfoot Spectral Cell Sorter (Thermo Fisher Scientific).

iii. Lentiviral Packaging and Transduction

To package the lentiviruses of the 110 MHC expression constructs (pHAGE-EF1a-MHC-UBC-NAT), MHC-null HEK293T cells were plated at 75% confluency in 96 wells and transfected using jetPRIME® transfection reagent (Polyplus, Illkirch, France). Individual MHC expression constructs were mixed with packaging plasmids (pREV/pTAT/pVSVG/pGAGPOL) and incubated with jetPRIMER reagent according to the manufacturer's protocol, and DMEM medium was added at 24 hours post-transfection. Viral supernatants were harvested 48 hours after transfection and used for transduction of the 110 MHCs in a 96-well-based array format.

To package the lentiviruses of control constructs, Lenti-X cells (Takara Bio USA, Mountain View, CA) were plated at 75% confluency and transfected using jetPRIME® transfection reagent (Polyplus, Illkirch, France). Expression constructs were mixed with packaging plasmids (pREV/pTAT/pVSVG/pGAGPOL) and incubated with jetPRIME® reagent according to the manufacturer's protocol, and Opti-Pro SFM medium was added at 24 hours post-transfection. Viral supernatants were harvested 48 hours after transfection and were concentrated using either Vivaspin 20 centrifugal concentrators or Vivaflow 50 cassettes (Sartorius, Bohemia, NY).
All viral transduction involving cell lines derived from HEK293T cells was done with polybrene (4 μg/ml).
iv. Co-Culture for Alloreactivity Profiling

Pan T cells (CD3⁺) were engineered as described above and frozen. On Day 0, T cells were restimulated in upright T25 flasks with 1.0E+6 T cells, 20.0E+6 irradiated PBMCs, recombinant human IL-2, and CD3 monoclonal antibody (OKT3) [0.1 ug/mL, eBioscience]. Half the volume of media was exchanged on Days 2, 4, 5, and 6 with RPMI 1640 supplemented with 10% heat-inactivated fetal bovine serum [FBS], 100 IU/mL penicillin, 100 μg/mL streptomycin, and recombinant human IL-2 [50 U/mL, PeproTech, Cranbury, NJ]. Cells were harvested for assay on Day 7.

The assay was performed in triplicates. On Day 5, target cells in the 96-well array were passaged and seeded in 384-well plates. On Day 6, engineered CD8+ T cells expressing the recombinant TCR MAGE-A1-1479 or untransduced control T cells were added at an effector to target (E:T) ratio of 5:1 and incubated with target cells for 48 hours. Target cell numbers were measured over time using IncuCyte® by measuring the number of NucLight™ Red-positive cells. Cell inhibition at 48 h by the TCR on each MHC in the assay was calculated as 1-(Cell doubling [Incubated with TCR MAGE-A1-1479 expressing T cells]/Cell doubling [Incubated with untransduced control T cells]).

v. Generation of TCR MAGE-A1-1479-Expressing CD8 T Cells for Safety Screens

CD8⁺ T cells expressing MAGE-A1 1479 as described above were thawed and restimulated (further expanded) by co-culturing T cells with irradiated (60 grays) allogeneic PBMCs in the presence of 0.1 μg/mL anti-CD3 (OKT3, eBioscience) and 50 U/mL recombinant IL-2 (Peprotech) in fresh T cell medium (RPMI 1640 supplemented with 10% heat-inactivated fetal bovine serum (FBS), 100 IU/mL penicillin, 100 μg/mL streptomycin, recombinant human IL-2 [50 U/mL, PeproTech]) in G-REX® 100 flasks (Wilson Wolf). 50 U/mL recombinant IL-2 was added to expanding cells every other day until day 6. On day 6, half of culture medium was replaced with fresh T cell medium containing 50U/mL recombinant IL-2. Cells were used for screens on Day 7.

vi. Peptide Library Design for Safety Screen

The human genome-wide peptide library was generated by tiling across the human genome coding sequences spanning all proteins of the human genome with overlapping 90-mer amino acid tiles. The tiles were synthesized on a silicon chip (Twist Bioscience) and cloned into a lentivirus expression vector.

vii. Library Virus Packaging and Tittering for Safety Screen

To generate peptide library-expressing reporter cells, peptide library constructs were first packaged using Lenti-X™ cells (Takara Bio USA, Mountain View, CA). Briefly, the Lenti-X™ cells were plated at 75% confluency in a CellBIND® polysyrene CellSTACK® 5-stack chamber (Corning), and transfected using jetPRIME® transfection reagent (Polyplus, Illkirch, France). Peptide libraries were mixed with packaging plasmids (pREV/pTAT/pVSVG/pGAGPOL) and incubated with the jetPRIME® reagent according to the manufacturer's protocol. Opti-Pro™ SFM medium (LifeTech) was added at 24 hours post-transfection. Viral supernatants were harvested 48 hours after transfection and were concentrated using Vivaflow® 50 cassettes (Sartorius, Bohemia, NY). Lentiviral titer was determined by puromycin colonies formation using Lenti-X™ cells using serial dilution of viral supernatant. To quantify viral titers, puromycin resistance colonies were selected 48 hours post-transduction. Pruomycin resistance colonies were visualized by crystal violet staining and counted. Titer was calculated as the colony forming unit as TU/ml using the formula: TU/ml=Number of puromycin resistance colonies×dilution factor×1000.

Reporter cells were generated by knocking endogenous HLA-A/B/C out in HEK293T cells using CRISPR and engineered to express a granzyme-activated infrared fluorescent protein (IFP) and a granzyme-activated scramblase (Ferretti et al. (2020) Immunity 53:1095-1107). Reporter cells for screens were engineered to express all HLA-A*02:01.

Reporter cells were transduced with lentiviral particles to express the PEP LIB V2 PLUS library at an MOI of 5 in complete DMEM (1×DMEM supplemented with 10% fetal bovine serum, 100 IU/mL penicillin, 100 μg/mL streptomycin). This library is comprised of >600,000 constructs (tiles) comprised of 90 amino acids each, representative of the human proteome. The day after transduction, complete DMEM media was refreshed and cells were maintained in culture or frozen in complete DMEM supplemented with 10% DMSO until needed.
viii. Co-Culture and Enrichment of Granzyme-Killed Cells

On day 7 of T cell expansion, T cells were added to library-transduced reporter cells at and E:T ratio of 1:1 and incubated at 37° C. for four hours. After incubation, all cells were harvested by trypsinization and centrifugation, cells were resuspended in 1× Annexin V binding buffer (Miltenyi Biotec) and centrifuged. Cells were resuspended with Annexin V magnetic microbeads (Miltenyi Biotec) in 1× Annexin V binding buffer (1 mL microbeads in 9 mL Annexin V binding buffer per 1×10⁹ total cells) and incubated at room temperature for 15 minutes. Cells were washed with 5× volume of Annexin V binding buffer and centrifuged. Cells were resuspended in Annexin V binding buffer and then divided over 2 “megareps” and filtered using a 70 μM cell strainer (Corning). Annexin V-labeled cells were positively selected using an AutoMACS Pro (Miltenyi Biotec). The elution of each “megarep” was further divided over four “replicates” for a total of 8 technical replicates per screen. IFP⁺ cells were sorted using a MoFlo Astrios EQ cell sorter (Beckman Coulter) and stored in DNA/RNA Shield™ (Zymo Research) for subsequent analysis.

ix. Next-Generation Sequencing (NGS) and Data Analysis

Genomic DNA was extracted using GeneJET™ Genomic DNA Purification Kit (Thermo Fisher Scientific, Waltham, MA) and prepared for NGS sequencing using two rounds of PCR amplification. In brief, the first round PCR amplified the peptide cassette and the second round added sequencing adapter and sample indexes before sequencing using an Illumina NextSeq™ instrument (Illumina, San Diego, CA).

Nucleotide sequences were mapped to individual nucleotide tiles. The proportion of read counts for each tile was calculated for each screen replicate (n=8) and for the input for each pool of transduced reporter cells, and enrichments of each tile were calculated by dividing the proportion of the tile in the screen replicate by the proportion of the tile in the input library. A modified geometric mean of the enrichment of an identical tile across the 8 screen replicates was used to identify reproducible screen hits.

d. Safety Evaluation of TCR MAGE-A1-1479 on Healthy Human Primary Cells

Cancer cell lines were cultured in the media described in Table 5.

TABLE 5
Cancer cell lines and their culture media

| Cancer Cell | |
|---|---|
| Lines | Culture Medium |
| CaSki | RPMI-1640 + 10% HIFBS + 1X Pen/Strep |
| Loucy | RPMI-1640 + 10% HIFBS + 1X Pen/Strep |
| U266B1 | RPMI-1640 + 20% HIFBS + 1X Pen/Strep |

i. Healthy Human Primary Cells Expressing Putative Off-Targets of TCR MAGE-A1-1479

Primary cells from healthy donors were thawed and cultured in the media described in Table 7 as per manufacturer's instructions.

TABLE 7
Healthy human primary cells and their culture media

| Primary Cells | Cell lot # | Culture medium |
|---|---|---|
| Human Umbilical Vein | 474Z035 | Endothelial Cell |
| Endothelial | | Growth Medium 2 |
| Cells (HUVEC) | | (Ready-to-use) |
| Growth Medium 2 | | |
| Human Pulmonary | 474Z024.2 | Fibroblast Growth |
| Fibroblasts (HPF) | | Medium 2 (Ready-to-use) |
| Human Small Airway | 467Z033, | Airway Epithelial Cell |
| Epithelial Cells | 467Z025.2 | Growth Medium |
| (HSAEpC) | | (Ready-to-use) |
| Human Bronchial | 469Z016 | Airway Epithelial |
| Epithelial | | Cell Growth |
| Cells (HBEpC) | | Medium (Ready-to-use) |
| Normal human | 451Z014.1 | Keratinocyte |
| epidermal | | Growth Medium 2 |
| keratinocytes (NHEK) | | (Ready-to-use) |
| Hepatocytes | HH1052, | UPCM-Universal |
| | HH1165 | Primary Cell |
| | | Plating Medium |
| Human small intestinal | 201457 | Epithelial Cell |
| epithelial cells (HSIEpC) | | Growth Medium |
| Human bronchial smooth | 0000613768, | SmGM-2 Smooth Muscle Cell |
| muscle cells (HBSMC) | 21TL104462 | Growth Medium-2 BulletKit |

ii. Cytokine Assay for Safety Evaluation of TCR-MAGE-A1-1479

HLA-A*02:01⁺MAGE-A1⁺ U266B1 cells were used as a positive control and HLA-A*02:01⁺MAGE-A1⁻ CaSki or Loucy cells were used as a negative control. 24 hours prior to co-culture assays, CaSki cells were detached from their culture flasks using TrypLE reagent, washed with media and seeded in 96-well flat-bottom culture plates at a density of 50,000 cells/well and allowed to adhere overnight. U266B1 and Loucy cells were washed on the day of co-culture and seeded in the 96-well flat-bottom culture plates at the same density. 24 hours prior to co-culture assays, HUVECs, HPFs, HSAEpCs, HBEpCs, HBSMCs, HSIEpCs and NHEKs were detached from their culture flasks using the DetachKit (PromoCell, Germany), washed and plated in their respective media at a density of 25,000 cells/well. Hepatocytes were thawed the day prior to co-culture as per the manufacturer's recommendation and plated at 56,000 cells/well and allowed to attach overnight. The following day, the primary target cells were pulsed with 100 ng/ml of MAGE-A1 peptide for 2 hours or left un-pulsed in their respective media. Following pulsing, wells were gently washed three times with target cell media. TCR-MAGE-A1-1479 or NTD cells were then added at a density of 50,000 cells/well in complete T cell media without cytokines. Supernatants were collected 24 hours post-co-culture and frozen at −80 C. Supernatants were thawed and analyzed for IFN-γ levels by loading on a Simple Plex Human IFN-gamma (3rd Gen) Cartridge (ProteinSimple, San Jose, CA) using the Ella instrument.

e. Materials

TABLE 8

| | |
|---|---|
| Cell lines | Lenti-X: Takara Bio USA, 632180 |
| | HEK293T cells: ATCC, CRL-3216 |
| | U266B1: ATCC, CRL-TIB-196 |
| | NCI-H1703: ATCC CRL-5889 |
| | A-375: ATCC CRL-1619 |
| | SK-MEL-5: ATCC HTB-70 |
| | CaSki: ATCC, CRL-1550 |
| | Loucy: ATCC, CRL-2629 |
| Healthy | Human Umbilical Vein Endothelial |
| human | Cells (HUVEC) Growth |
| primary | Medium 2: PromoCell, A-12991 (Lot # 474Z035) |
| cells | Human Pulmonary Fibroblasts (HPF): |
| | PromoCell, C-12360 (Lot # 474Z024.2) |
| | Human Small Airway Epithelial Cells (HSAEpC): |
| | PromoCell, C-12642 (Lot # 467Z033, 467Z025.2) |
| | Human Bronchial Epithelial Cells (HBEpC): |
| | PromoCell, C-12640 (Lot # 469Z016) |
| | Normal Human Epidermal Keratinocytes (NHEK): |
| | PromoCell, C-12003 (Lot # 451Z014.1) |
| | Hepatocytes: In Vitro ADMET Laboratories, |
| | 82006 (Lot # HH1052, HH1165) |
| | Human Bronchial Smooth Muscle Cells (HBSMC): |
| | Lonza, CC-2576 (Lot # 0000613768, 21TL104462) |
| | Human Small Intestinal Epithelial Cells (HSIEpC): |
| | iXCells Biotechnologies, 10HU-237 (Lot # 201457) |
| Media and | X-VIVO 15, serum-free hematopoietic cell medium, |
| supplements | with L-Glutamine, gentamycin |
| | and phenol red: Lonza, 04-418Q |
| | Human male AB serum (heat-inactivated): |
| | Sigma Aldrich, H3667-100ML |
| | Penicillin streptomycin: Gibco, 15149-122 |
| | GlutaMAX supplement: Fisher Scientific, 35050061 |
| | RPMI-1640 medium: ATCC, 30-2001 |
| | Fetal bovine serum (FBS), heat-inactivated: |
| | Gibco, A3840102 |
| | RPMI medium 1640 (1x) [+] 4.5 g/L D-glucose, |
| | [+] 2.383 g/L HEPES buffer, [+] L-glutamine, |
| | [+] 1.5 g/L sodium bicarbonate, |
| | [+] 110 mg/L sodium pyruvate: Gibco, A10491-01 |
| | Endothelial Cell Growth Medium 2 (Ready-to-use): |
| | PromoCell, C-22011 |
| | Fibroblast Growth Medium 2 (Ready-to-use): |
| | PromoCell, C-23020 |
| | Small Airway Epithelial Cell Growth Medium |
| | (Ready-to-use): PromoCell, C-21070 |
| | Airway Epithelial Cell Growth Medium (Ready-to-use): |
| | PromoCell, C-21060 |
| | Keratinocyte Growth Medium 2 (Ready-to-use): |
| | PromoCell, C-20011 |
| | SmGM-2 Smooth Muscle Cell Growth Medium-2 |
| | BulletKit: Lonza, CC-3182 |
| | Epithelial Cell Growth Medium: iXCells |
| | Biotechnologies, MD-0041 |
| | UCRM ™-Universal Cryopreservation Recovery |
| | Medium, 50 mL: In Vitro ADMET Laboratories, 81015 |
| | UPCM ™-Universal Primary Cell Plating Medium, |
| | 50 mL: In Vitro ADMET Laboratories, 81016 |
| | DetachKit: PromoCell, C-41220 |
| | TrypLE ™ Express: Thermo Fisher |
| | Scientific, 12605-010 |
| | Penicillin streptomycin: Gibco, 15149-122 |
| | RPMI-1640 medium: ATCC, 30-2001 |
| | Fetal bovine serum (FBS), heat-inactivated: |
| | Gibco, A3840102 |
| | IMDM: ATCC, 30-2005 |
| | DMEM medium (1X) [+] 4.5 g/L D-glucose, |
| | [+] L-glutamine, [+] 3.7 g/L sodium |
| | bicarbonate: Thermo Fisher Scientific, 11965084 |
| | DMEM (1x) [+] 4.5 g/L D-glucose, [+] L-glutamine, |
| | [−] Sodium Pyruvate: Gibco, 11965 |
| | OptiPRO SFM medium: Thermo Fisher |
| | Scientific, 12309019 |
| | EMEM medium: ATCC, 30-2003 |
| Buffers | DPBS (no Ca2+/Mg2+): Gibco, 14190-122 |
| | EasySep buffer: StemCell Technologies, 20144 |
| Cytokines | Recombinant human IL-2: Sigma Aldrich, 11147528001 |
| | Recombinant human IL-7: R&D Systems, 207-IL-025 |
| | Recombinant human IL-2: PeproTech, 200-02 |
| | Recombinant human IL-7: R&D Systems, 207-IL-005 |
| | Recombinant human IL-15: R&D Systems, 247-ILB-005 |
| Kits | Simple Plex Human IFN-gamma (3rd Gen) Cartridge: |
| | ProteinSimple, SPCKB-PS-002574 |
| | EasySep ™ Human T Cell Isolation Kit: StemCell |
| | Technologies: 17951 |
| | Ella simple plex kit for 32 samples: ProteinSimple, |
| | SPCKB-PS-003027 |
| Peptides | MAGEA1₂₇₈₋₂₈₆ (KVLEYVIKV (SEQ ID NO: 1)): |
| | Genscript |
| Antibodies | CD3 monoclonal antibody (OKT), functional |
| and staining | grade: eBioscience 16-0037-81 |
| reagents | Anti-human CD8α PerCp-Cy5.5 (clone: HIT8a): |
| | BioLegend: 300924 |
| | Anti-human CD4 APC-Cy7 (Clone: OKT4): |
| | BioLegend: 317418 |
| | Anti-human CD56 PE (clone: 5.1H11): |
| | BioLegend: 362508 |
| | Anti-human CD57 PE (clone: HCD57): |
| | BioLegend: 322312 |
| | Anti-human CD34 Alexa Fluor 488: (Clone: |
| | QBEND/10): R&D Systems, FAB7227G |
| | Anti-human HLA-A2 PE (clone: BB7.2): |
| | BioLegend, 343306 |
| | Anti-human TCRα/β PE-Cy7 (clone: IP26): |
| | BioLegend: 306720 |
| | Anti-human HLA-A,B,C APC Antibody: |
| | BioLegend, 311410 |
| | Fixable viability Dye eFluor 660 (APC Channel) |
| | Thermo Fisher Scientific: 65-0864-14 |
| | DAPI: Thermo Scientific: Thermo Scientific, 62248 |
| | Cytofix: BD Biosciences, 554655 |
| Dextramers | HLA-A*02:01 KVLEYVIKV (SEQ ID NO: 1) |
| | MAGEA1₂₇₈₋₂₈₆ dextramer: Immundex |
| CRISPR- | Alt-R ® S.p. Cas9 Nuclease V3 (Lot #0000417827): |
| Cas9 and | IDT, 1081059 |
| electro- | Alt-R ® CRISPR-Cas9 tracrRNA |
| poration | (Lot #0000415438): IDT, 1072534 |
| reagents | 4D-Nucleofector ™ Core Unit: Lonza, AAF-1002B |
| | 4D-Nucleofector ™ X Unit: Lonza, AAF-1002X |
| | SE Cell Line 4D-Nucleofector ™ X Kit L: |
| | Lonza V4XC-1012 |
| Tissue | G-REX ® 24-well plate: Wilson Wolf, 80192M |
| culture | G-REX ® 6-well plate: Wilson Wolf, 80240M |
| plates | VECELL ® 96-well plate: Cosmo Bio Co., |
| | VCL-V96WGPB-10-EX |
| | TC-treated 6-well plate: Corning Costar, 3506 |
| | TC-treated 24-well plates: Corning Costar, 3524 |
| | 96-well flat-bottom plates: Corning Costar, 3595 |
| | 384-well flat-bottom plates: Corning Costar, 3764 |
| | CellAdhere ™ Collagen I-Coated, |
| | 96-well flat-bottom plate: |
| | StemCell Technologies, 100-0366 |
| Packaging | Lenti-X GoStix Plus: Takara, 631281 |
| and | jetPRIME transfection reagent: |
| lentivirus | Polyplus-transfection, 114-75 |
| production | Vivaspin 20: Sartorius, VS2041 |
| reagents | Vivaflow 50 cassettes: Sartorius, VF05P4 |
| Other | ImmunoCult ™ Human CD3/CD28/CD2 T |
| reagents | cell activator: StemCell Technologies, 10970 |
| | Poly-L-ornithine solution 0.01%: |
| | Millipore-Sigma, P4957-50ML |
| | CryoStor CS10: StemCell Technologies, 07930 |
| | ViaStain AO/PI staining solution in PBS: |
| | Nexcelom Bioscience, CS2-0106-5mL |
| | Nourseothricin: GoldBio, N-500-1 |
| | Puromycin: Gibco, A11138-03 |
| | Mirus TransIT, Mirus Bio MIR 2704 |
| | Polybrene: EMD Millipore, TR-1003-G |
| | Incucyte ® NucLight Red Lentivirus |
| | Reagent (EF-1 Alpha |
| | Promoter, Puromycin selection), Sartorius, 4476 |

### Example 3:1676 MAGEA1₂₇₈₋₂₈₆ (KVLEYVIKV (SEQ ID NO: 1)) Specific TCRs were Identified

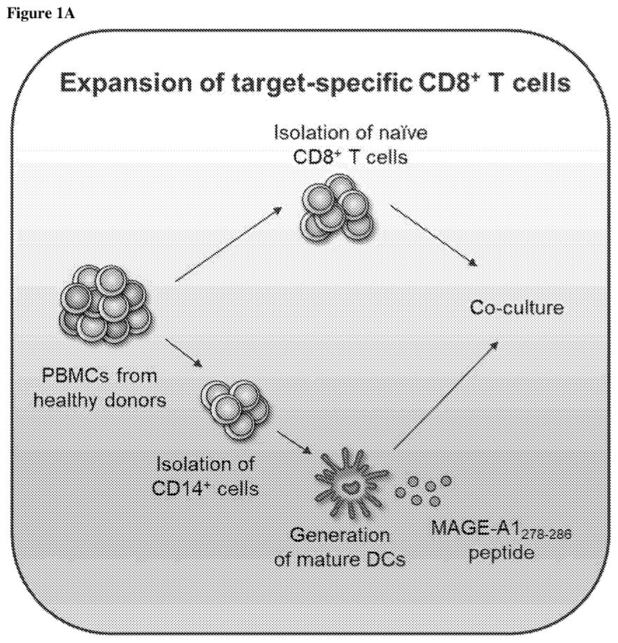

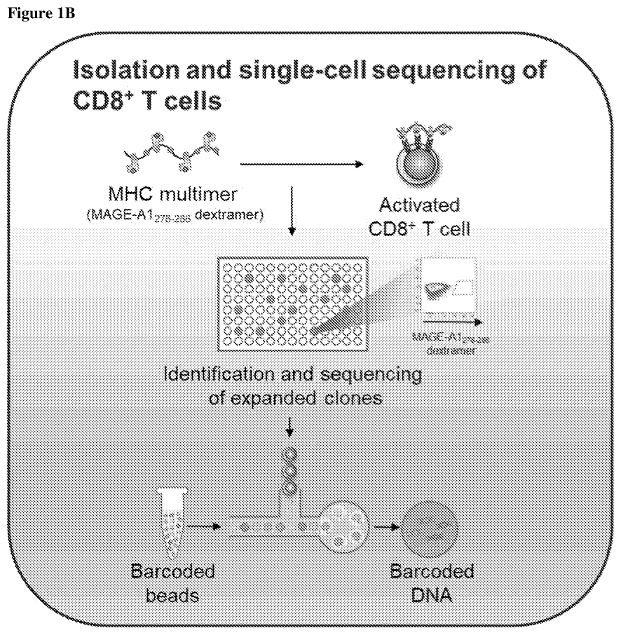

MAGE-A1 specific TCRs were identified and validated to HLA-A*02:1 restricted epitope KVLEYVIKV (SEQ ID NO: 1). The MAGEA1₂₇₈₋₂₈₆ (KVLEYVIKV (SEQ ID NO: 1)) peptide sequence has been described in U.S. Pat. No. 10,874,731, the content of which is incorporated by reference herein in its entirety. 1676 MAGEA1₂₇₈₋₂₈₆ (KVLEYVIKV (SEQ ID NO: 1)) specific TCRs were identified using a platform depicted in FIGS. 1A and 1B. Briefly, CD14+ monocytes were isolated from PBMCs of HLA-A*02:01 healthy donors on day-4 and differentiated to mature DCs. On day-1, naïve CD8 T cells were isolated from autologous PBMCs and rested overnight. Co-culture of naïve CD8 T cells and DCs was performed following 3 hours pulsing of DCs with 1 μg/mL MAGEA1₂₇₈₋₂₈₆ peptide as part of the multiplexed screens, followed by an 11-day cell expansion phase. Dextramer staining was performed with HLA-A*02:01-specific MAGEA1₂₇₈₋₂₈₆ (KVLEYVIKV (SEQ ID NO: 1)) dextramer to identify clones. DNA barcoded dextramers were used to isolate MAGEA1₂₇₈₋₂₈₆ specific cells. TCR alpha beta pairs were identified by 10× genomics platform.

### Example 4:30 Out of 500 TCRs were Selected by Multiple Rounds of VAYG Screens for Functional Assessment

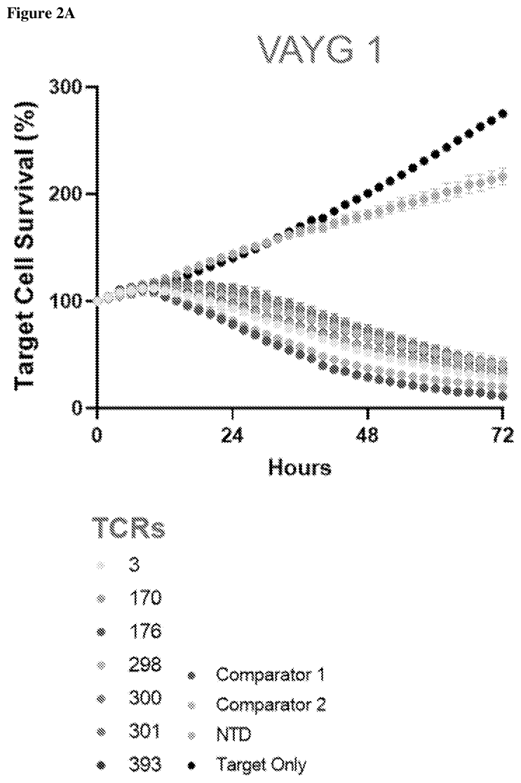

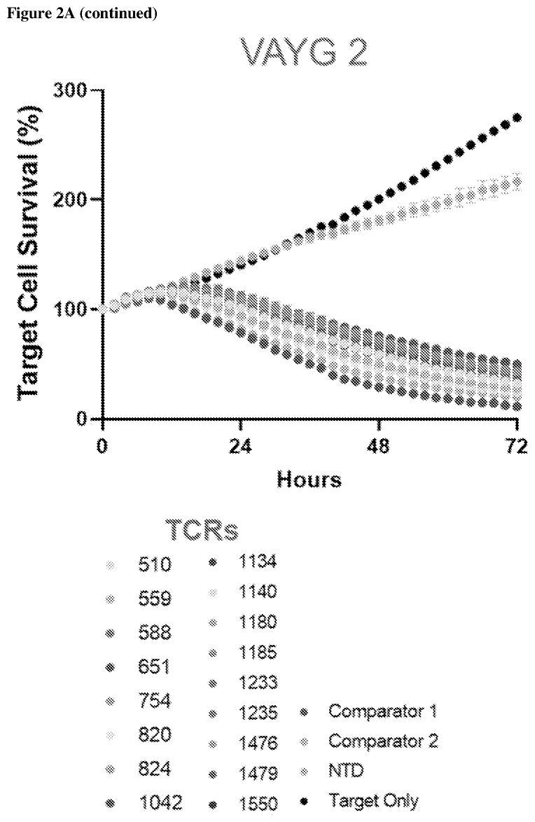

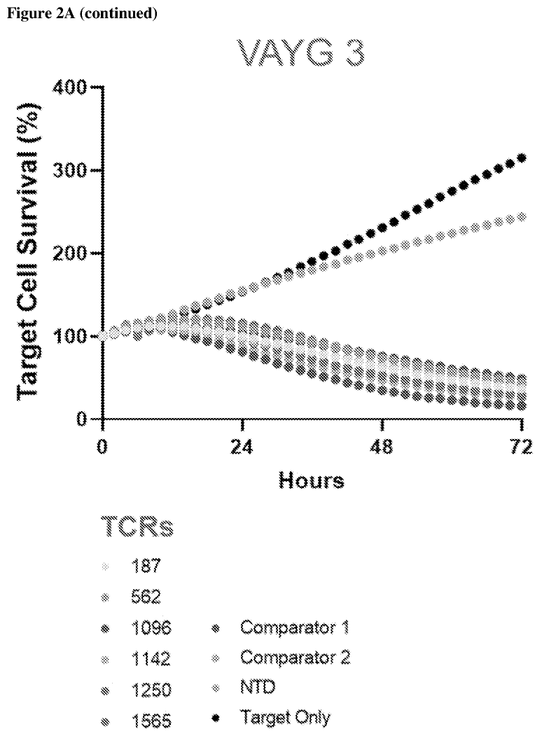

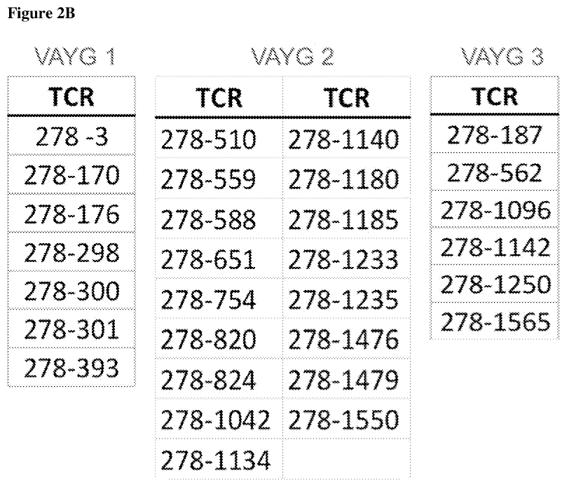

Pan-T cells were transduced to express 500 MAGEA1-specific TCRs individually. Engineered T cells were co-cultured with NucLight® Red labeled target cells such as NCIH1703 cells. Survival of the target cells was quantified by time-dependent imaging as a readout of T cell cytotoxicity. Survival curves for TCRs that showed potent cytotoxicity are shown (FIG. 2A) and TCR IDs listed (FIG. 2B). Non-transduced cells (NTD) and two comparator TCRs served as controls. 30 out of 500 TCRs listed in FIG. 2B were selected for further evaluation for surface expression.

### Example 5: Selection of MAGEA1₂₇₈₋₂₈₆ TCRs Based on Expression and Cytotoxic Function

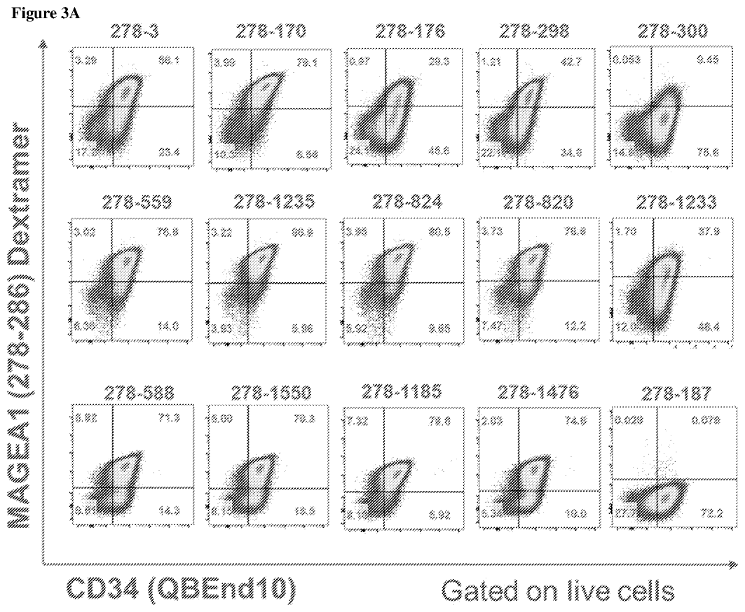

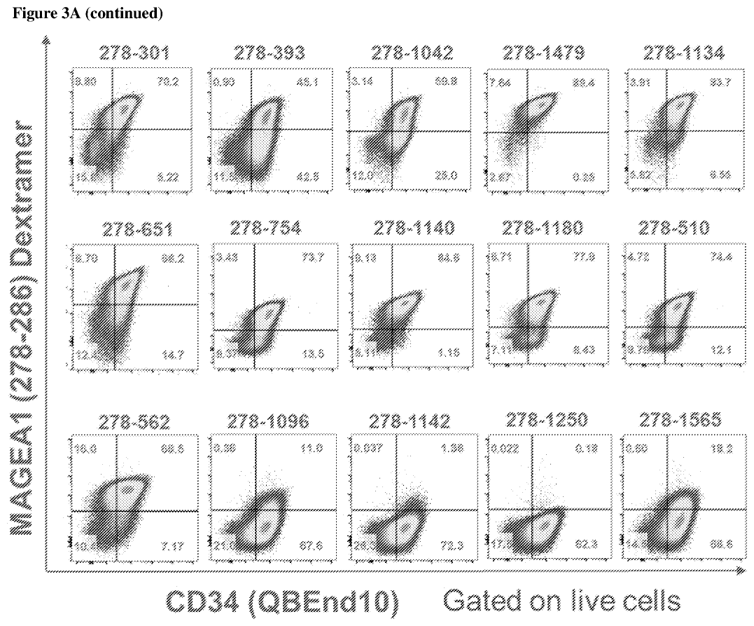

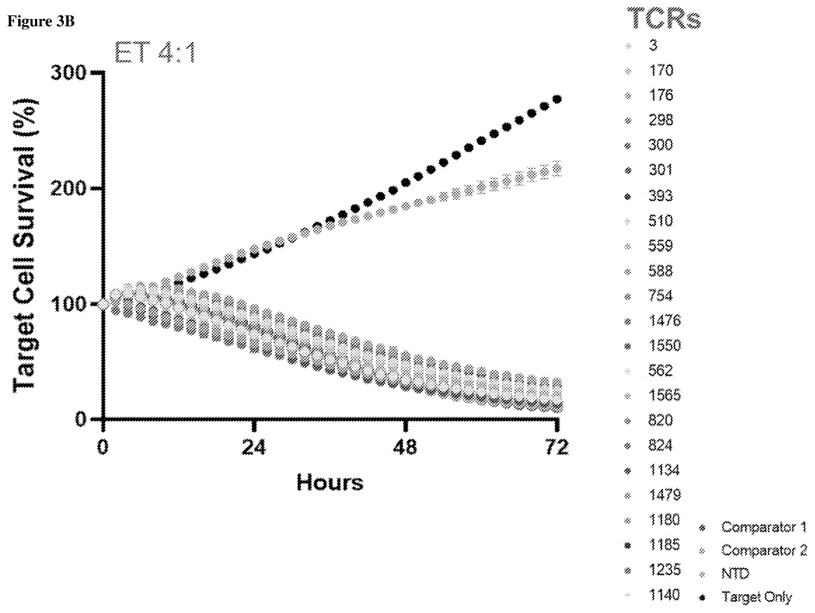

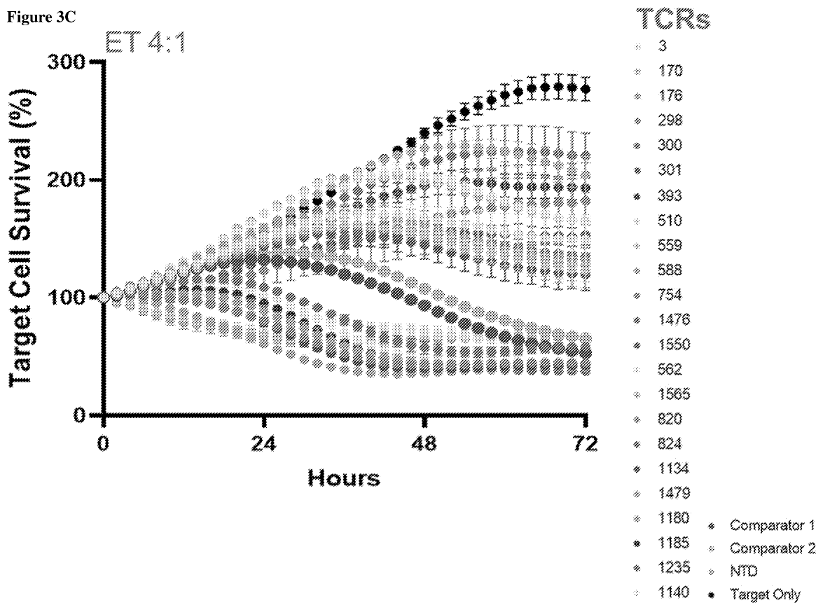

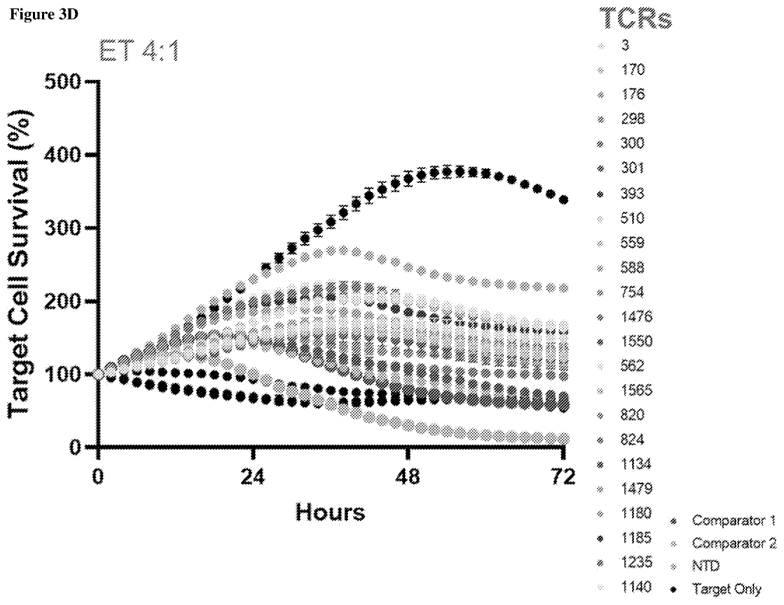

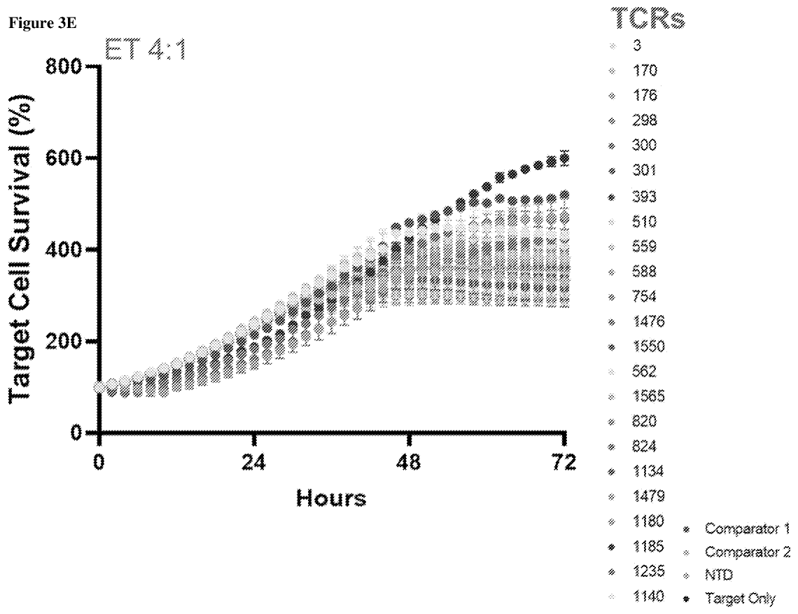

Pan-T cells from an HLA-A*02:01 positive healthy donor were transduced to express 30 MAGEA1₂₇₈₋₂₈₆ TCRs that were selected from the VAYG screens. Surface expression of the TCRs was assessed by MAGEA1₂₇₈₋₂₈₆ (KVLEYVIKV (SEQ ID NO: 1)) dextramer staining. 24 out of 500 TCRs tested were selected based on high surface binding of MAGEA1₂₇₈₋₂₈₆ dextramer and were evaluated further in an in vitro cytotoxicity assay where they were compared to the “comparator TCR”. Dot plots show surface expression of the 30 TCRs as assessed by A*02:01-specific MAGEA1₂₇₈₋₂₈₆ (KVLEYVIKV (SEQ ID NO: 1)) dextramer staining (FIG. 3A). Cytotoxic responses of these TCRs to HLA-A*02:01⁺ MAGEA1^{+/-} target cell lines (FIG. 3B) NCIH1703, (FIG. 3C) Hs936T and (FIG. 3D) A375 (FIG. 3E) HEK293T are shown. Engineered T cells were co-cultured with NucLight® Red labeled target cell lines at indicated E:T ratios, and their survival was quantified on an IncuCyte as a readout of cytotoxicity of the T cells.

### Example 6: Functional Evaluation of MAGEA1₂₇₈₋₂₈₆ TCRs

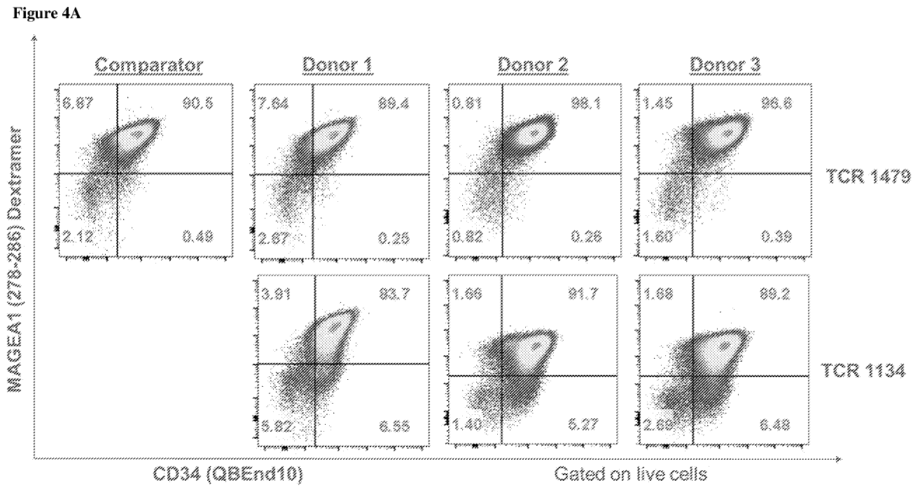

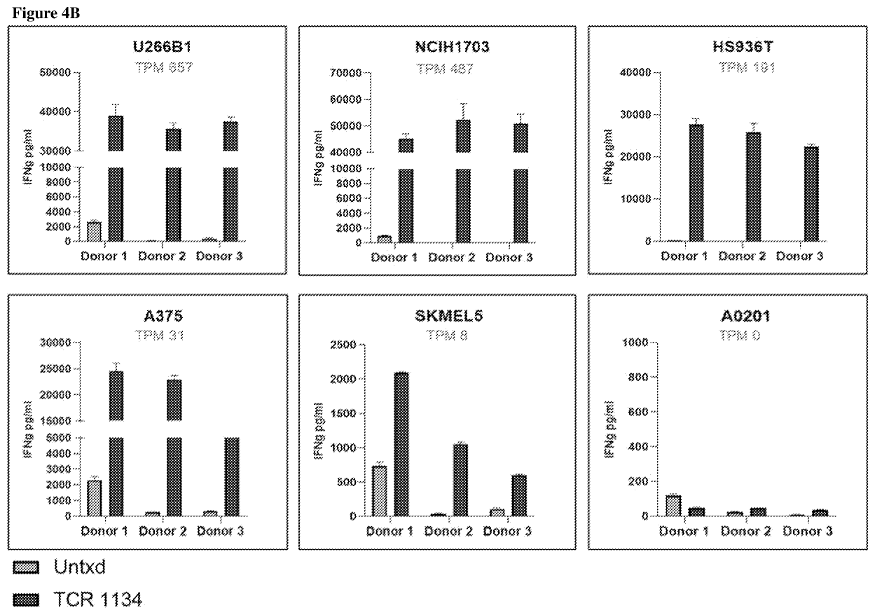

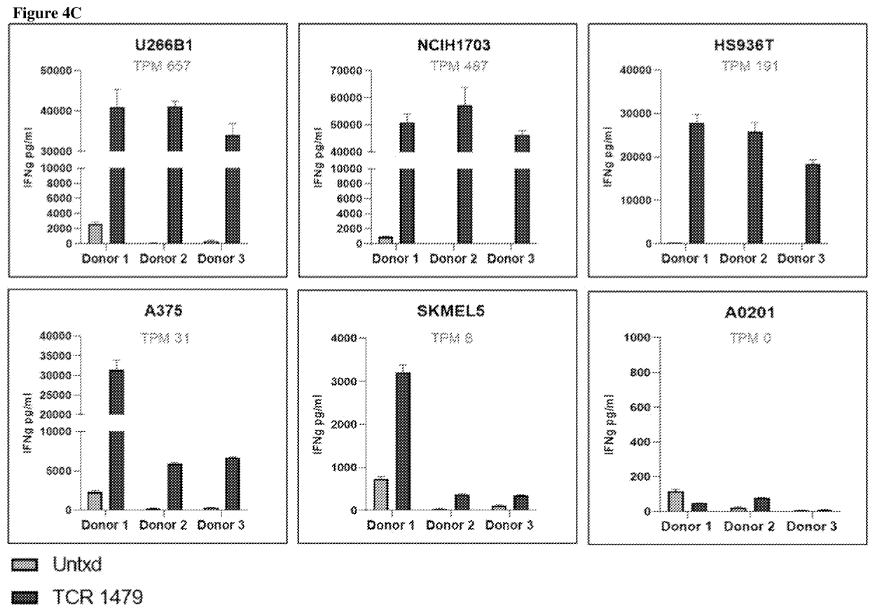

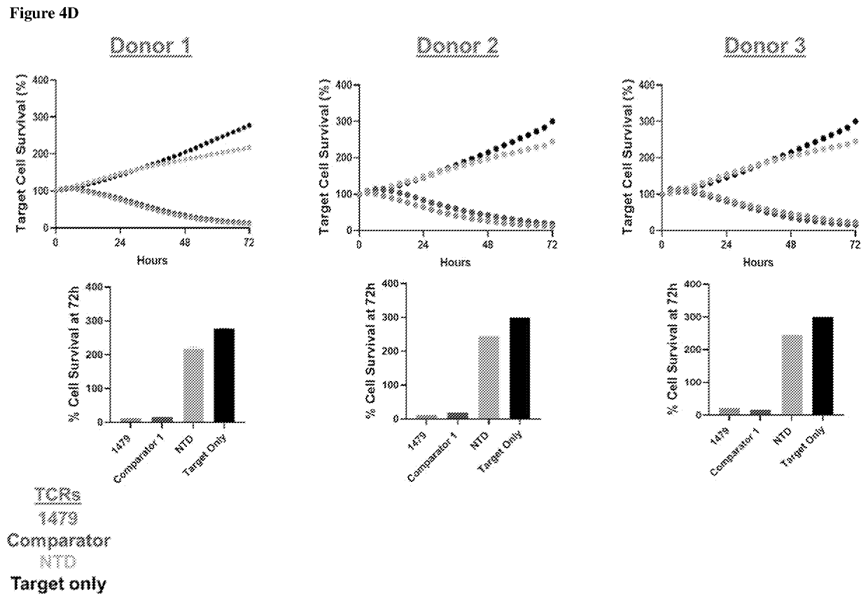

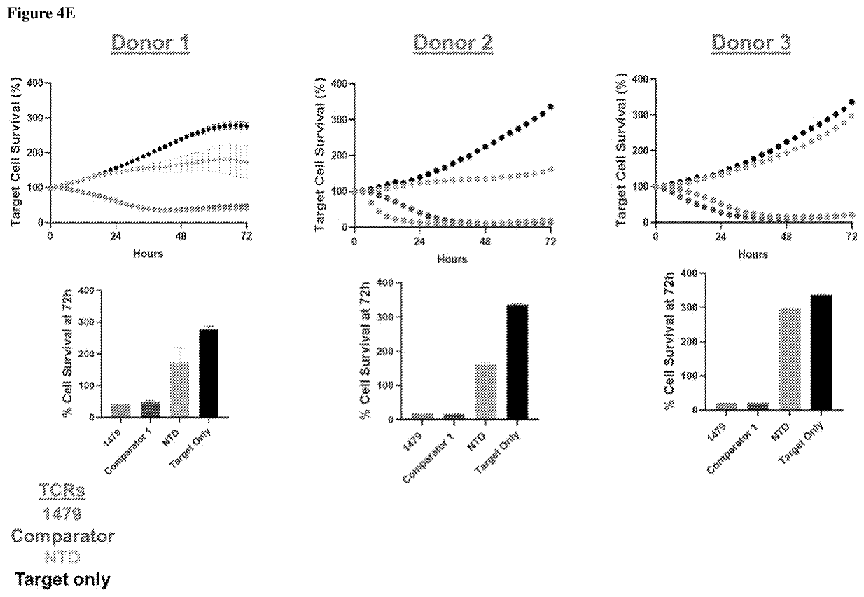

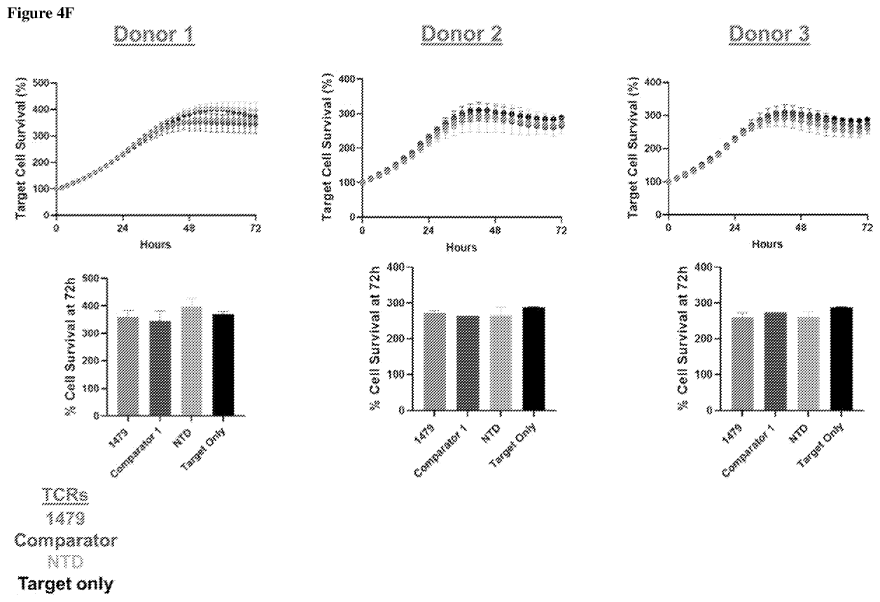

Pan T cells isolated from three HLA-A*02:01 positive healthy donor PBMCs were transduced to express MAGEA1₂₇₈₋₂₈₆ specific TCRs MAGE-A1-1134; MAGE-A1-1479 and the “comparator TCR” and assessed for functional responses to target cells positive and negative for MAGEA1 and HLA-A*02:01. Dot plots show expression of the MAGEA1₂₇₈₋₂₈₆ specific TCRs as assessed by A*02:01-specific MAGEA1₂₇₈₋₂₈₆ (KVLEYVIKV (SEQ ID NO: 1)) dextramer staining (FIG. 4A). Functional responses measured from IFN-Y secretion in co-culture supernatants at 24 h (E:T 1:1) of the MAGEA1₂₇₈₋₂₈₆ specific TCRs to HLA-A*02:01⁺ MAGEA1^{+/-} target cell lines are shown (FIGS. 4B and 4C). Functional response of the lead MAGEA1₂₇₈₋₂₈₆-specific TCR 1479 to HLA-A*02:01⁺ MAGEA1⁺ target cell lines NCIH1703 (FIG. 4D), Hs936T (FIG. 4E), and the HLA-A*02:01⁺ MAGEA1 negative control cell line HEK293T (FIG. 4F) are shown. Engineered T cells were co-cultured with NucLight® Red labeled target cell lines at indicated E:T ratios, and their survival was quantified on an IncuCyte® as a readout of cytotoxicity of the T cells.

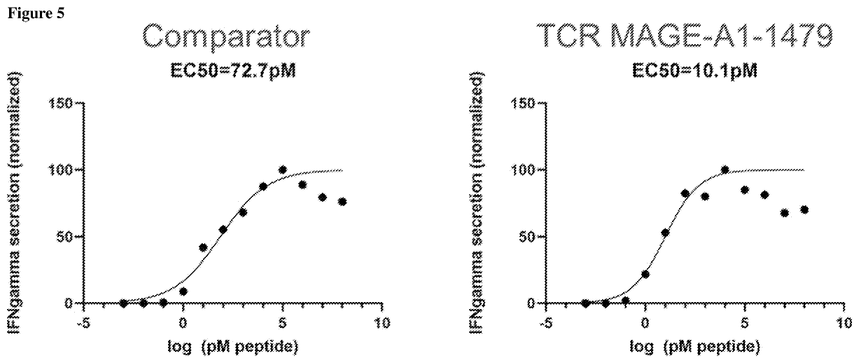

MAGE-A1-1479 or “comparator TCR”-expressing pan-T cells were tested for their reactivity to HLA-A*02:01+T2 cells pulsed with the MAGE-A1 (KVLEYVIKV (SEQ ID NO: 1)) peptide. IFNγ secretion in culture supernatants was used as a read out of MAGE-A1-1479's reactivity to peptide-pulsed target cells (FIG. 5).

### Example 7: Genome-Wide Off-Target Screen for TCR MAGE-A1-1479

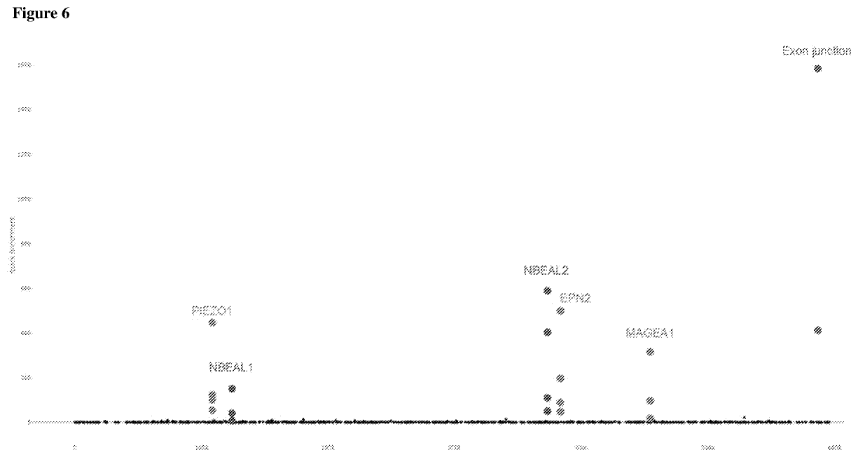

The genome-wide screen data of TCR MAGE-A1-1479 identified five potential off-targets in a screen of >600,000 protein fragments spanning every wildtype (w.t.) human protein (FIG. 6). The screen was designed to overpredict off-targets by overexpressing 90-aa protein fragments, which are more efficiently processed than full-length proteins. Putative off-targets were identified by gene names. The annotated exon-junction derived from a bioinformatically predicted exon skipping event in the gene RNASEH2B. This exon-skipped form of RNASEH2B transcript was not observed in any RNAseq data from biological human samples in either GTEX or TCGA databases.

### Example 8: TCR MAGE-A1-1479 Showed No Alloreactivity to 109/110 MHCs Tested

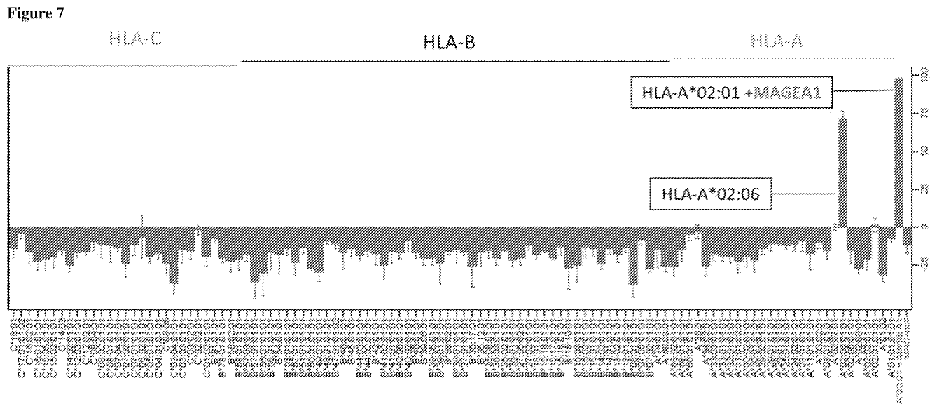

TCR MAGE-A1-1479-expressing pan T cells or untransduced control T cells were co-cultured with MHC-null HEK293T cells re-expressing one of the 110 most frequently encountered Class I MHCs in the US population for 48 hours. A positive control consisting of HEK293T cells expressing both HLA-A*02:01 and the MAGE-A1 ORF which contains the epitope (KVLEYVIKV (SEQ ID NO: 1)) was included in the screen. The inhibition on target cell growth by the TCR MAGE-A1-1479-expressing pan T cells relative to that by the untransduced control T cells was measured after 48 hour of co-culture as a readout of the reactivity of the TCR to allogeneic MHC molecules. The % target cell inhibition for each allele relative to the positive control is indicated (FIG. 7).

### Example 9: MAGE-A1-1479 Shows No Reactivity to Healthy Human Primary Cells

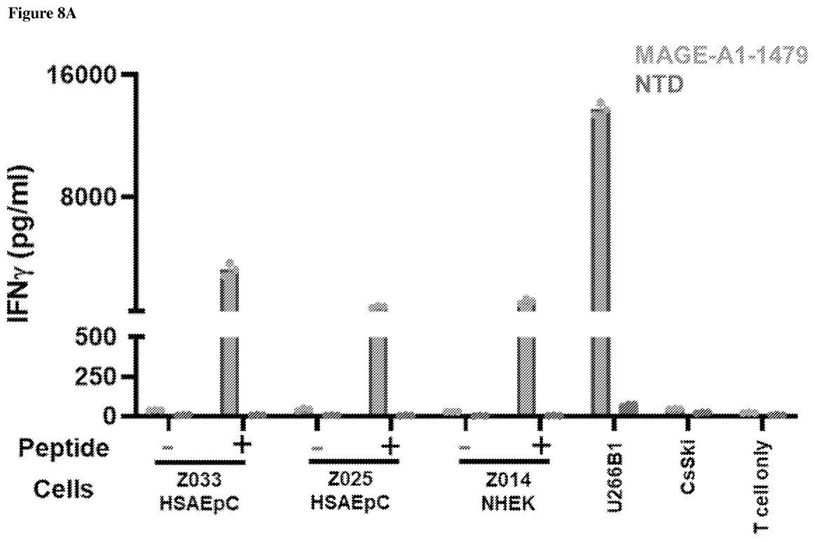

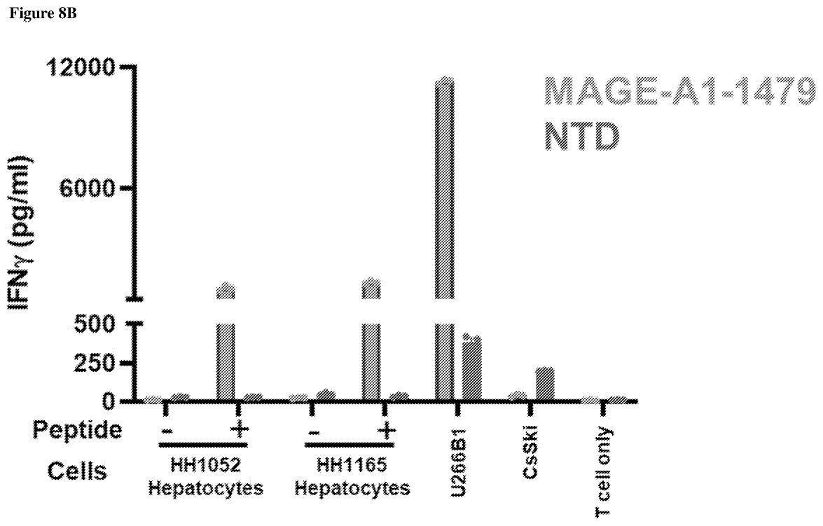

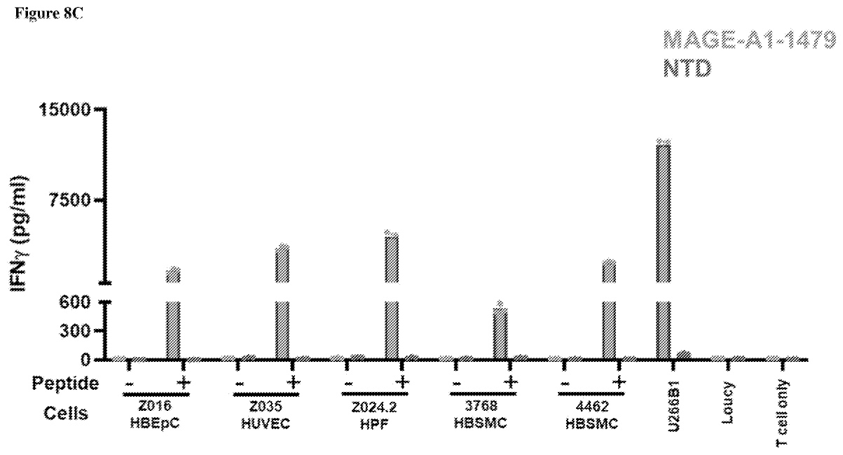

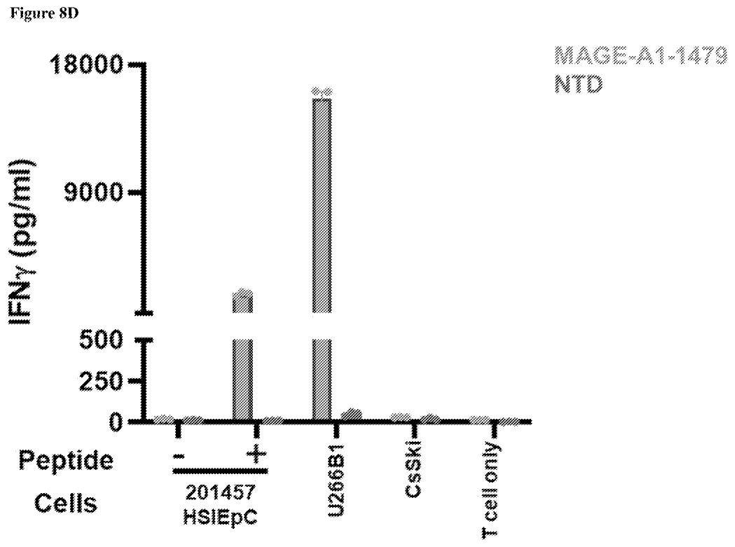

MAGE-A1-1479-expressing pan-T cells or NTD cells were tested for their reactivity to primary cells derived from healthy HLA-A*02:01+ human donors naturally expressing off-targets identified in the genome wide safety screen (FIGS. 8A-8D). Target cells were pulsed with the MAGE-A1 (KVLEYVIKV (SEQ ID NO: 1)) peptide or left unpulsed, and co-cultured with MAGE-A1-1479 or NTD cells. IFNγ secretion in culture supernatants was used as a read out of MAGE-A1-1479's reactivity to target cells. HLA-A*02:01⁺MAGE-A1⁺ U266B1 cells were used as a positive control and HLA-A*02:01⁺MAGE-A1 CaSki or Loucy cells were used as negative controls.

### Example 10: Further Confirmatory Characterization of TSC-204-A0201 Action

The following representative Examples 10-15 further confirm the data and results presented in working examples 1-8 and continue to be based, in part, on the recognition that adoptive cell transfer with genetically engineered T cells holds great promise for treating solid tumors. Patients positive for particular HLA alleles of interest, such as HLA-A*02:01, are amenable to treatment with TCRs recognizing epitopes of a given target presented by such HLAs, such TSC-204-A0201.

For representative Examples 10-15, process-representative TSC-204-A0201 TCR-T cells (e.g., helper (CD4⁺, now CD8⁺/CD4⁺) and cytotoxic (CD8⁺) T cells) were engineered by transposon/transposase-mediated gene delivery of vector pNVVD136 (i.e., pNVVD136_TSC-204-A02_TCR-1479_MSCV-TCR-1479-CD8-EF1α-dnTGFbRII-DHFR) (unless otherwise indicated), to express (1) a recombinant TCR (e.g., the recombinant TCR specific to the MAGE-A1-derived peptide KVLEYVIKV (SEQ ID NO: 1) presented on HLA-A*02:01), (2) recombinant CD8α and CD8β co-receptors to maximize the efficacy of the therapeutic product, (3) a CD34-derived epitope tag fused on the N-terminus of CD8α to facilitate tracking of engineered cells in vitro and in vivo, (4) a mutated form of dihydrofolate reductase (DHFRdm) protein to facilitate enrichment of engineered cells during the manufacturing process, and (5) a dominant negative type II TGFβ receptor (DN-TGFβRII) to further address tumor microenvironment-mediated immune suppression. Such cells can be manufactured using known techniques and, for the present example, were generated through isolation of peripheral blood mononuclear cells (PBMC) from a fresh apheresis product, delivery of transposase mRNA and vector transposon npDNA by electroporation, T cell activation and culture, engineered cell enrichment via addition of methotrexate (MTX) in culture medium (selective growth advantage of engineered cells conferred by DHFRdm expression from transposon vector), cell washing to remove MTX, culture expansion, and cryopreservation.

TSC-204-A0201 TCR-T cells were analyzed by flow cytometry to characterize the cell composition. The clinically representative TSC-204-A0201 TR-T cells contain engineered (i.e., CD34⁺) helper (CD4⁺, now CD4⁺/CD8⁺) and cytotoxic (CD8⁺) T cells. The engineered helper and cytotoxic T cells express the recombinant TCR able to recognize the MAGE-A1-derived peptide KVLEYVIKV (SEQ ID NO: 1) bound to HLA-A*02:01 as demonstrated by the binding of the relevant dextramer. Engineered TCR-T cells also express DN-TGFβRII.

Functional data were generated to characterize the mechanism of action of TSC-204-A0201. The data demonstrate that clinically representative TSC-204-A0201 TCR-T cells from at least 3 independent batches (subjects) react to the MAGE-A1 derived peptide KVLEYVIKV (SEQ ID NO: 1) in a dose-dependent manner when presented by target cells on the HLA-A*02:01 MHC in peptide pulse experiments. Three representative, independent batches of TCR-T cells generated from different donors were tested for their reactivity against their cognate peptide/MHC. As described further below, TSC-204-A0201 TCR-T cells were co-cultured with peptide pulsed T2 cells, a cell line unable to present endogenous peptide on class I MHC molecules due to a deficiency in the peptide transporter TAP (Steinle & Schendel (1994) Tissue Antigens 44:268-270). T2 cells were pulsed with a titration of the MAGE-A1-derived peptide KVLEYVIKV (SEQ ID NO: 1) and TCR reactivity was determined by measuring the amount of IFN-γ secreted by the TCR-T cells as a function of the dose of peptide pulsed on the target cells. This study confirmed that the TSC-204-A0201 specifically reacts to cells presenting the MAGE-A1-derived peptide KVLEYVIKV (SEQ ID NO: 1) presented on HLA-A*02:01.

When encountering a target cell naturally expressing MAGE-A1 and HLA-A*02:01, TSC-204-A0201 TCR-T cells engage in the secretion of inflammatory cytokines and of granzyme B and mount a proliferative response detected both in the engineered cytotoxic and helper T cell subpopulations. The TCR-T cells eventually kill these target cells naturally presenting the MAGE-A1-derived peptide on HLA-A*02:01. Here, a panel of target cancer cell lines naturally expressing MAGE-A1 and HLA-A*02:01 was used in co-culture assays with the 3 independent batches of TCR-T cells to evaluate the biological outcomes regarding TSC-204-A0201 engagement. Untransfected (UTF) control T cells obtained from matching donors were used as negative controls. Untransfected (UTF) control T cells were also produced from the isolated PBMCs used to generate each batch of TSC-204-A0201 process-representative material tested. The UTF cells did not undergo the electroporation step, nor the purification step, but were similarly activated and cultured as the TSC-204-A0201 TCR-T cells. Target cells positive for MAGE-A1 expression but negative for HLA-A*02:01 were used as additional negative controls. Multiple readouts were used to evaluate the functional engagement of the TCR-T cells, including: (1) secretion of inflammatory cytokines (IFN-γ, TNF-α and IL-2) and granzyme B in the co-culture supernatant was evaludated after 20-24 hours of c-culture; (2) proliferation of the engineered T cells (both CD4⁺ and CD4⁻) was assessed after 3.5 days of co-culture; and (3) selective cytotoxic activity was examined over 72 hours of co-culture.

In addition, the target-dependent function of TSC-204-A0201 is insensitive to physiological levels of TGFβ (e.g., active even in the presence of TGFβ, an immuno-suppressive cytokine typically observed in the microenvironment of solid tumors), a function provided by the expression of the DN-TGFβRII.

Materials and methods for preparing effector T cells and target cells, as well as setting up co-cultures, are described herein. T cells were thawed in a 37° C. water bath and washed once with cytokine-free T cell medium to remove cryopreservation reagents prior to being resuspended in complete T cell medium. T cells were then seeded in a G-REX® 6-well plate at a density of 1E6 live cells/mL and allowed to recover in a humidified incubator at 37° C. and 5% CO₂ for 16-24 hours prior to co-culturing. On the day of co-culture, T cells were harvested, washed, and resuspended in cytokine-free T cell medium at the desired cell density. Similarly, target cell were prepared similarly.

Similarly, target cells (Table 9) were prepared. For example, cancer cell lines were thawed in a 37° C. water bath and washed once with cell culture medium to remove cryopreservation reagents. Cells were subsequently resuspended in cell culture medium and cultured following standard procedures in 75 cm² flasks (adherent target cells) or G-REX® wells (suspension cells) in a humidified incubator at 37° C. and 5% CO₂. Cells were kept at a sub-confluent state, in the exponential growth phase and passaged once or twice a week as needed. The cancer cell lines were maintained in culture at least one passage, and no longer than 4 weeks prior to the initiation of the co-culture with T cells.

In addition, co-cultures were prepared. Adherent target cells were plated one day before setting up the co-culture. For the Incucyte based cytotoxicity assay, target cells were plated in 100 μL of their respective medium in 96 well flat bottom plates at 5E3 cells per well (A101D, AU565) or 7E3 cells per well (NCIH1703, HS936T, SW1271) to achieve a target cell density of ~1E4 cells per well after 20-24 hours incubation at 37° C., 5% CO₂. Seeding densities were adapted according to the variable growth rates of cell lines. For cytokine and proliferation assays, target cells were plated in 96 well flat bottom plates at 2.5E4 cells per well (A101D, AU565) or 3.5E4 cells per well (NCIH1703, HS936T, SW1271) to achieve a target cell density of ~5E4 cells per well after 20-24 h incubation at 37° C. 5% CO₂. Non-adherent cells (i.e., U266B1, Loucy and T2) were plated the day of initiation of the co-culture using assay-specific seeding densities.

The following discussion further details certain experiments and results introduced above.

a. Flow cytometric analysis of process-representative TSC-204-A0201 TCR-T cellsFirst, the cellular composition of TSC-204-A0201 TCR-T cells was examined by flow cytometry. Test sample cells were thawed, washed, resuspended in complete T cell medium, plated on a G-REX® plate, and incubated overnight at 37° C. prior to being processed for staining as described above. After overnight recovery, TCR-T cell test articles and their untransfected controls were washed, labeled with antibodies from the “Dextramer Panel” (Table 9) and acquired. Unstained cells, single stain controls and FMO controls were also prepared.

TABLE 9
Cancer cell, media, flow cytometry “Dextramer Panel”, and T cell proliferation detection
reagents, and appendix reagents, respectively
Cancer cell reagents

| | | | MAGE-A1 | HLA-A*02:01 |
|---|---|---|---|---|
| Target cell line | Indication | Source | (TPM) | (HLA-A TPM) |
| NCIH1703 | Lung | ATCC | + (182.08) | + (817.32) |
| SW1271 | Lung | ATCC | + (84.02) | + (385.75) |
| HS936T | Melanoma | ATCC | + (69.86) | + (901.57) |
| A101D | Melanoma | ATCC | + (136.79) | − (342.72) |
| AU565 | Breast | ATCC | + (72.46) | + (44.95) |
| U266B1 | Myeloma | ATCC | + (241.7) | + (321.72) |
| LOUCY | T lymphoblast | ATCC | − (0.02) | + (1578.5) |
| T2 | Lymphoma | ATCC | − (ND) | + (ND) |

Media reagents

| | | | V stock | Vendor (Catalog |
|---|---|---|---|---|
| Media | Cell lines | Components | (mL) | Number) |
| Cytokine-free T cell | Primary T | X-VIVO 15 | 1000 | Lonza #04-418Q |
| medium | cells | Human Serum (Heat- | 50 | Sigma-Aldrich #H3667-100 |
| | (co-culture) | Inactivated) | | |
| | | Pen Strep | 10 | Gibco #15149-122 |
| | | GlutaMAX (100x) | 10 | FisherScientific #35050061 |
| Complete T cell | Primary T | Cytokine-free T cell | 200 | n/a |
| medium | cells | medium | | |
| | (thawing, | Human IL-2 (5 mg/mL) | 1 | Sigma-Aldrich #11147528001 |
| | resting) | Human IL-7 (10 mg/mL) | 0.1 | R&D Systems #207-IL-025 |
| RPMI-based medium | NCIH1703 | RPMI | 500 | ATCC30-2001 |
| 10% FBS | AU565 | Fetal Bovine Serum | 50 | Thermofisher Scientific |
| | LOUCY | | | #A3840001 |
| | | Penicilin/Streptomycin | 10 | Thermofisher Scientific |
| | | | | #15149-122 |
| RPMI-based medium | U266B1* | RPMI | 500 | ATCC30-2001 |
| 15% FBS | | Fetal Bovine Serum | 75 | Thermofisher Scientific |
| | | | | #A3840001 |
| | | Penicilin/Streptomycin | 10 | Thermofisher Scientific |
| | | | | #15149-122 |
| RPMI-based medium | SW1271 | RPMI | 500 | ATCC30-2001 |
| 20% FBS | | Fetal Bovine Serum | 100 | Thermofisher Scientific |
| | | | | #A3840001 |
| | | Penicilin/Streptomycin | 10 | Thermofisher Scientific |
| | | | | #15149-122 |
| DMEM-based | HS936T, | DMEM | 500 | Thermofisher Scientific |
| medium 10% FBS | A101D | | | #11965092 |
| | | Fetal Bovine Serum | 50 | Thermofisher Scientific |
| | | | | #A3840001 |
| | | Penicillin/Streptomycin | 10 | Thermofisher Scientific |
| | | | | #15149-122 |
| IMDM-based medium | T2 | IMDM | 500 | ATCC#30-2005 |
| 20% FBS | | Fetal Bovine Serum | 100 | Thermofisher Scientific |
| | | | | #A3840001 |
| | | Penicillin/Streptomycin | 10 | Thermofisher Scientific |
| | | | | #15149-122 |

Flow cytometry “Dextramer panel” reagents

| Antibody | Clone | Vendor | Fluorochrome | Catalog Number |
|---|---|---|---|---|
| near-IR | N/A | Invitrogen | Near-IR (APC- | L34976A |
| fluorescent | | | Cy7) | |
| reactive dye | | | | |
| CD34 | QBEND/10 | Invitrogen | Biotinylated | MA5-16924 |
| Streptavidin | N/A | BioLegend | AF488 | 40235 |
| CD4 | RPA-T4 | BioLegend | PE-Cy7 | 300538 |
| CD8b | 2ST8.5H7 | BioLegend | BV421 | 742390 |
| TGFβRII | W17055 | BioLegend | PE | 399704 |
| MAGEA1/HL | TCR1479(MAGEA1) | IMMUDEX | APC | Custom (HLA- |
| A-A*02:01 | | | | A*0201/KVLEYVI |
| Dextramer | | | | KV (SEQ ID NO: |
| | | | | 1)) |

T cell proliferation detection reagents

| Reagent | Clone | Vendor | Fluorochrome | Catalog Number |
|---|---|---|---|---|
| Human Trustain | N/A | Biolegend | N/A | 422302 |
| Fc Block | | | | |
| Far Red Fixable | N/A | Thermofisher | Far Red (APC) | L10120 |
| live dead dye | | Scientific | | |
| CD34 | QBEND/10 | Thermofisher | Biotinylated | MA5-16924 |
| | | Scientific | | |
| Streptavidin | N/A | BioLegend | Alexa488 | 405235 |
| CD4 | RPA-T4 | BioLegend | PE-Cy7 | 300538 |
| CD3 | UCHT1 | BD Bioscience | Brilliant Violet 650 | 563852 |

Appendix reagents

| Media | Vendor | Catalog Number |
|---|---|---|
| X-VIVO 15 | Lonza | 04-418Q |
| Human Serum (Heat-Inactivated) | Sigma-Aldrich | H3667-100 |
| HI FBS | Gibco | Q38400-01 |
| FBS, heat-inactivated | Thermofisher Scientific | A3840001 |
| GlutaMAX (100x) | Fisher Scientific | 35050061 |
| Penicillin-Streptomycin | Gibco | 15140-122 |
| X-VIVO 15 | Lonza | 04-418Q |
| Human Serum (Heat-Inactivated) | Sigma-Aldrich | H3667-100 |
| HI FBS | Gibco | Q38400-01 |
| Cell Line | Vendor | Catalog Number |
| T2 (174xCEM.T2) | ATCC | CRL-1992 |
| RPMI1640 | ATCC | 30-2001 |
| NCIH1703 | ATCC | CRL-5889 |
| HS936T | ATCC | CRL-7687 |
| SW1271 | ATCC | CRL-2177 |
| A101D | ATCC | CRL-7898 |
| AU565 | ATCC | CRL-2351 |
| U266B1 | ATCC | TIB-196 |
| Loucy | ATCC | CRL-2629 |

| Marker | Fluorophore | Vendor | Clone | Cat# |
|---|---|---|---|---|
| CD34 | Biotinylated | Thermofisher | QBEND/10 | MA5-16924 |
| | | Scientific | | |
| Streptavidin | Alexa488 | BioLegend | n/a | 40235 |
| HLA- | APC | IMMUDEX | TCR1470(MAGEA | WB03474 AP |
| A*0201/KVLEYVIKV | | | 1) | 150 |
| (SEQ ID NO: 1) | | | | |
| (dextramer) | | | | |
| CD3 | Brilliant Violet 650 | BD Bioscience | 563852 | UCHT1 |
| CD4 | PE-CY7 | BioLegend | RPA-T4 | 300512 |
| CD8b | Brilliant Violet 421 | BD | 2ST8.5H7 | 742390 |
| TGF-B Receptor II | PE | BioLegend | W17055E | 399704 |
| LIVE/DEAD | near-IR(APC-Cy7) | Thermofisher | n/a | L10119A |
| | | Scientific | | |
| LIVE/DEAD | Far Red (APC) | Thermofisher | n/a | L10120 |
| | | Scientific | | |
| UltraComp eBeads Plus | n/a | Thermofisher | n/a | 01-3333-42 |
| Comp Beads | | Scientific | | |
| ArC negative | n/a | Thermofisher | n/a | (B)A10346 |
| | | Scientific | | |
| ArC positive | n/a | Thermofisher | n/a | (A)A10346 |
| | | Scientific | | |
| CellTraceTM Violet | CellTrace Violet | Thermofisher | n/a | C34557 |
| proliferation kit | | Scientific | | |

| Reagent | Vendor | Catalog Number |
|---|---|---|
| DPBS/Modified | Cytivia | SH30028.03 |
| DPBS without CaCl2, MgCl2 | Thermofisher Scientific | 14190-144 |
| Tryple E | Thermofisher Scientific | 1260-08 |
| EasySepBuffer | STEMCELL | 20144 |
| | Technologies | |
| ViaStain AOPI Staining Solution | Nexcelom Bioscience | CS2-0106-25mL |
| DMSO (Dimethyl Sulfoxide), anhydrous | Thermofisher Scientific | L10119B |
| DMSO (Dimethyl Sulfoxide), anhydrous | Thermofisher Scientific | D12345 |
| Reconstitution buffer 2 (0.1% BSA in PBS) | RD Systems | RB02 |
| Reconstitution buffer 4 (4 mM HCL, 0.1% BSA) | RD Systems | RB04 |
| Interleukin-2, recombinant human (rhIL-2) | Sigma-Aldrich | 1147528001 |
| Interleukin-7, recombinant human (rhIL-7) | RD Systems | 207-IL/CF |
| TGFβ1, recombinant human | RD Systems | 7754-BH-025/CF |
| Simple Plex Cartridge for 72 samples, | Biotechne, Protein | SPCKB-PS-002574 |
| 1 analyte Human-IFNg (3rd Gen) | Simple | |
| Simple Plex Cartridge for 4 | Biotechne, Protein | SPCKC-PS-003027 |
| analytes/32 samples, human | Simple | |
| GRANB, IFNG (3rdGen), IL2, TNFA | | |
| Human Trustain FcX Fc Receptor | Biolegend | 422302 |
| blocking reagent | | |
| BD CytofixTM Fixation Buffer | BD Bioscience | 554655 |

Data acquisition was performed on a Cytoflex S. Compensation was performed automatically with CytExpert software. Data analysis was performed with FlowJo v7.6.5, Excel 2010.

The following description provides the cell gating strategy. Briefly, cells were gated from the FSC versus SSC dot plot and singlets distinguished from the aggregates using FSC-Area versus FSC-Height plot. Viable cells were identified using the Near-Infrared Live-Dead versus FSC-Area plot. Subpopulations were gated from the viable cells and evaluated, including, CD4⁺ (Helper T cells), CD4⁺/CD8⁺ (Engineered Helper T cells), CD4⁻/CD8⁺(Cytotoxic T cells), and TGFβRII⁺/CD34⁺ (engineered TCR-T cells expressing DN-TGFβRII). Expression of the therapeutic TCR was probed by positive detection for dextramer (MAGEA-A1-derived peptide KVLEYVIKV (SEQ ID NO: 1) bound to HLA-A*02:01) in either CD34⁺, CD4⁺ or CD4⁻ cells.

Flow cytometric analysis confirmed that TSC-204-A0201 TCR-T cells from five different batches contained engineered helper cells (CD4⁺, now CD4⁺/CD8⁺), residual non-engineered helper T cells (CD4⁺/CD8⁻), and cytotoxic (CD8+) T cells. The percentage of non-engineered CD4⁺/CD8⁻ helper T cells ranged from 9.13% to 17.3%. Engineered helper T cells expressed the exogenous CD8αβ co-receptor and were characterized as CD8⁺/CD4⁺. The TSC-204-A0201 TCR-T cell material presented between 27.40% and 44.20% of these engineered helper T cells and the percentage of cytotoxic T cells (CD8+/CD4) ranged from 37.6% to 61.5%.

The engineered T cells were confirmed to express the recombinant TCR specific to the MAGEA-1-derived peptide KVLEYVIKV (SEQ ID NO: 1) bound to HLA-A*02:01, as revealed by dextramer binding, as well as the CD34 epitope. No dextramer positive or CD34 positive population was detected in untransfected (UTF) T cells from matched donors. The percentage of the dextramer⁺/CD34⁺ population ranged from 38.4% to 61.2%.

Both the engineered CD4⁻ and CD4⁺ T cells were able to bind the dextramer, confirming that functional TCRs were found on the surface of both the helper and cytotoxic T cells. The percentage of Dextramer⁺/CD4⁻ populations ranged from 26.9% to 50.3%, and the percentage of Dextramer⁺/CD4⁺ ranged from 14.5% to 22%.

Engineered T cells making up TSC-204-A0201 material also expressed the DN-TGFβRII, as evidenced by a marked increase in the TGFβRII⁺ signal in the CD34⁺ population when compared to non-engineered CD34⁻ cells.

b. pMHC Dose-Dependent Function of the Recombinant TCR Expressed by TSC-204-A0201 TCR-T Cells

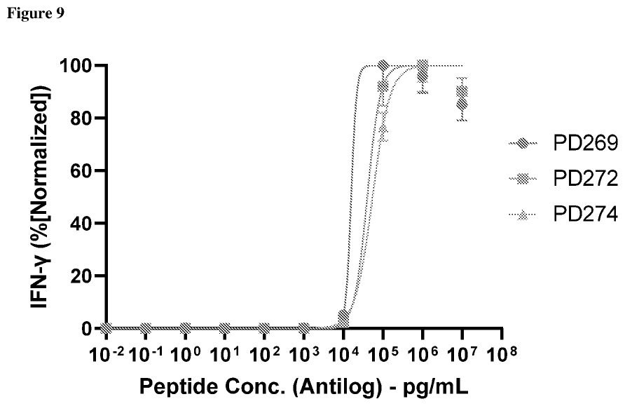

FIG. 9 shows reactivity of TSC-204-A0201 TCR-T cells from three independent donors against their cognate peptide/MHC as determined using a peptide pulse assay.

Specifically, on the day of co-culture, T2 cells were harvested and washed once with T2 cell peptide loading serum-free medium. T2 cells were pulsed with a 9-points serial dilution of the MAGE-A1-derived peptide KVLEYVIKV (SEQ ID NO: 1) (final concentration ranged from 10 μg g/mL-0.1 μg/mL). Peptide-loaded cells were washed, and cell density was adjusted to 5E5 live cells/mL in T2 culture medium. After thawing and overnight recovery, TSC-204-A0201 TCR-T cells were harvested, washed with cytokine-free T cell medium and resuspended at 5E5 live cells/mL as described above. T cell suspension (5E4 total T cells) were plated in U-bottom 96 well plates. Peptide-pulsed T2 cells were plated on top of the TCR-T cells (5E4 total T2 cells) for an E:T of 1:1. After 20 hours co-culture at 37° C. 5% CO₂, supernatants were collected and processed for IFN-γ analysis on the Protein Simple ELLA (automated ELISA platform) according to the manufacturer's instructions.

Data acquisition was performed on Protein Simple ELLA. The raw data were exported and graphed in GraphPad Prism (v5.02). The IFN-γ secretion (pg/mL) in response to peptide-pulsed T2 cells was plotted as a function of peptide dose. The data were normalized with 0% based on the smallest mean in each data set (n=3) and 100% based on the largest mean in each data set. Results were presented as percentages. A nonlinear regression fit was used to display a “normalized response” model.

As shown in FIG. 9, the three batches of process-representative TSC-204-A0201 TCR-T cells reacted to the KVLEYVIKV (SEQ ID NO: 1) peptide presented on HLA-A*02:01 in a dose-dependent manner and detectable IFN-γ secretion/TCR-T activity was observed at a peptide dose of 1 ng/mL and saturated at ~100 ng/mL. Altogether, these data confirmed that TSC-204-A0201 TCR-T cells specifically react to the MAGE-A1-derived peptide KVLEYVIKV (SEQ ID NO: 1) presented on HLA-A*02:01.

c. TSC-204-A0201 TCR-T Cells Exhibit Target-Dependent Cytokine Production

Target-dependent cytokine induction and granzyme B secretion of TSC-204-A0201 TCR-T cells were evaluated. Target-dependent induction of granzyme B secretion, as well as secretion of the pro-inflammatory cytokines IFN-γ, IL-2 and TNF-α, was assessed after 20 hours co-culture of the three batches of TSC-204-A0201 TCR-T cells with a panel of cancer cell lines selected for their endogenous expression of MAGE-A1 as well as HLA-A*02:01. A negative control target cell line, A101D, which is positive for MAGE-A1, but negative for HLA-A*02:01, was included in the assay. Donor-matched untransfected (UTF) control T cells were also included in the experiment and served as an additional negative control.

Specifically, adherent target cells were plated one day before setting up the co-culture, as described above, and effectors were prepared for the co-culture as described above. To initiate the co-culture, 5E4 effectors in 100 μL T cell medium were added to the target cells, resulting in an E:T of 1:1. After 20 hours co-culture at 37° C. 5% CO₂, supernatants were collected and processed for IFN-γ analysis on the Protein Simple ELLA (automated ELISA platform) in conjunction with 4-plex (IFN-γ, TNF-α, IL-2 and Granzyme B) ELLA cartridges according to the manufacturer's instructions. Data acquisition was performed on Protein Simple ELLA. The raw data were exported and graphed in GraphPad Prism (v5.02).

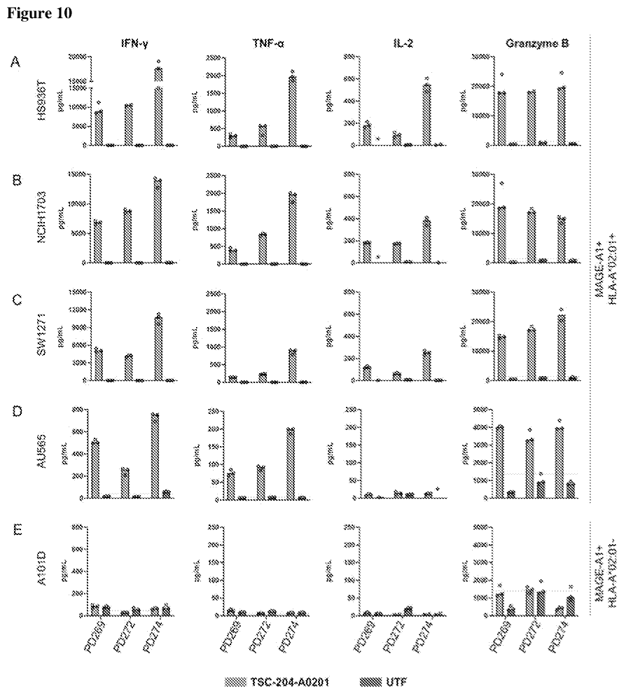

FIGS. 10A-10C show that cell lines HS936T, NCIH1703, SW1271 elicited robust induction of IFN-γ, TNF-α and granzyme B secretion from all batches of TSC-204-A0201 TCR-T cells tested. Induction of IL-2 secretion was also detected despite difficulties in measuring IL-2 levels when compared to other cytokines as it is a T cell mitogen that is not only produced by T cells, but also consumed by T cells.

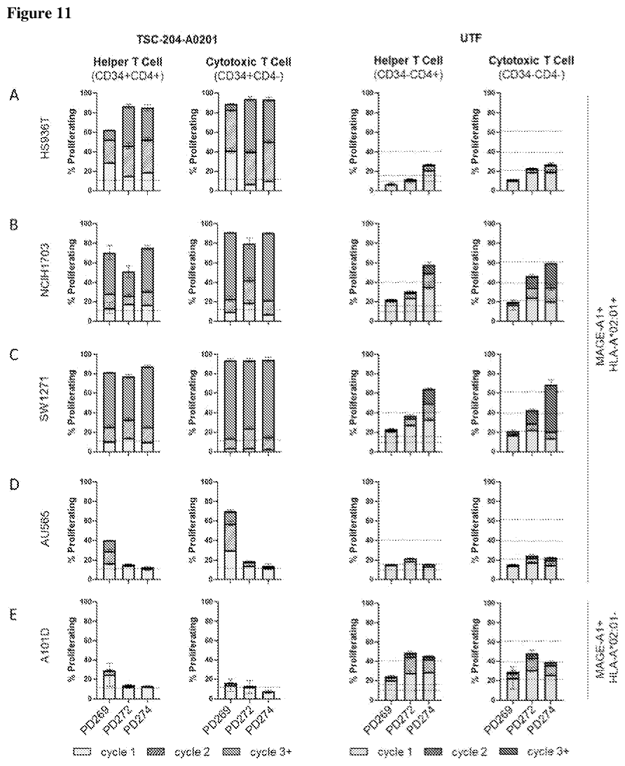

None of the batches of TSC-204-A0201 TCR-T cells secreted cytokine or Granzyme B upon co-culture with the negative control target cell line (the HLA-A*02:01-negative A101D cells), confirming the HLA selectivity for T cell engagement (FIG. 11E). Similarly, untransfected (UTF) control T cells from matched donors did not engage in cytokine or Granzyme B secretion when co-cultured with any of the MAGE-A1/HLA-A*02:01 positive target cells (FIG. 10). TSC-204-A0201 TCR-T cells cultured in the absence of any target cells also produced little to no cytokines and granzyme B (FIG. 10; baseline cytokine and Granzyme B secretion of TSC-204 TCR T cells indicated by dashed lines in the different panels).

Taken together, these data demonstrate that TSC-204-A0201 TCR-T cells operate in a target-dependent manner, reacting to target cell lines endogenously expressing MAGE-A1 and HLA-A*02:01 with robust cytokine and granzyme B secretion.

d. TSC-204-A0201 TCR-T Cells Proliferate in a Target-Dependent Manner

Target-dependent proliferation of the engineered T cells (both CD4+ and CD4−) was also assessed. Briefly, effectors were thawed and allowed to recover overnight as described above. To eliminate baseline proliferation induced by the T cell cytokines IL-2 and IL-7, effectors were washed once with cytokine-free T cell medium, reseeded in 6 well G-REX® plates at a concentration of 1-2E6 live cell/mL and incubated for an additional 20-24 hours in cytokine-free T cell medium. Adherent target cells were plated one day before setting up the co-culture as described above; non-adherent target cells (U266B1 and Loucy) were plated in 100 μL target cell medium in U-bottom plates at 5E4 live cells per well. Before initiating the co-culture, effectors were stained with CTV dye as follows: effectors were washed once with EasySep™ and stained for 7 minutes at room temperature with CTV dye diluted 1:2000 in EasySep™. After washing twice with T cell medium, 5E4 CTV labeled effectors in 100 μL T cell medium were added to the target cells to achieve an E:T of 1:1. Target and effectors were then co-cultured for 3.5 days at 37° C. 5% CO₂. At the end of the co-culture, effectors were transferred to a v-bottom plate and stained with the staining reagents (see Table 9 above). Data acquisition was performed on a Cytoflex S following SOP-PC-0001-Instrument SOP-Use and Maintenance of the Cytoflex. Compensation was performed with CytExpert software using single-color controls. Data analysis was performed with FlowJo v7.6.5, Excel 2010.

The following provides the cell gating strategy used. Briefly, cells were initially gated from the FSC-A/SSC-A plot and were separated from debris. Next, single cells were separated from aggregates using the FSC-Height versus FSC-Area plot. The target cells used for the co-culture were engineered to express Nuclight® Red, a fluorescent protein with a broad emission spectrum that is detectable both in the APC and the PE channel. Therefore, an APC versus CD3 plot was used to distinguish targets (APC⁺CD3⁻) from T cells (APC-CD3⁺). Since cells were stained with a far red live dead dye, dead cells appeared also as APC⁺ events and were gated out in the APC versus CD3 plot. A PE versus CD3 plot was then used to gate out any remaining target derived debris (i.e., PE⁺ events). Subsequently, transduced (i.e., CD34⁺) T cells were identified in a CD34 versus FSC-Area plot and were further separated into helper T cells (CD4⁺) and cytotoxic T cells (CD4⁻) in a CD3 versus CD4 plot. Because engineered CD4⁺ T cells express exogenous CD8aβ proteins, only the CD4 marker was used to distinguish helper (CD4⁺) and cytotoxic (CD4⁻) TCR-T cells in the flow cytometry analysis. Given that the events analyzed were initially gated on CD3⁺ T cells, the CD4⁻ fraction contains exclusively cytotoxic T cells. Dilution of the CellTrace® Violet dye was assessed in both the helper and cytotoxic T cell subsets using a CD3 versus CTV plot. TCR-T cells cultured in the absence of target, or in the presence of A101D (i.e., the HLA-A*02:01-negative target cell line) served as negative controls and were used to identify the position of the CTV peak of undivided cells. Additional gates were drawn to identify cells that had cycled once, twice, or up to 6 times.

FIG. 11 shows results of evaluation of target-dependent induction of proliferation. The panel of cancer cell lines used to evaluate target-dependent induction of cytokine and granzyme B secretion was also used to assess target-dependent induction of T cell proliferation. The three independent batches of process-representative TSC-204-A0201 TCR-T cells were labeled with CTV dye and co-cultured with the cancer cells for 3.5 days at an E:T of 1:1. Subsequently, proliferation was evaluated in transduced helper TCR-T cells (CD34⁺/CD4⁺) and transduced cytotoxic TCR-T cells (CD34⁺/CD4⁻).

Unstimulated TSC-204-A0201 TCR-T cells (i.e., TCR T cells cultured in the absence of target cells) displayed little base line proliferation (FIGS. 11D and 11E, baseline proliferation is indicated by dashed lines). On the other hand, co-culture of TSC-204-A0201 TCR-T cells with three of the target cell lines expressing MAGE-A1 and HLA-A*02:01 (i.e., HS936T, NCIH1703 and SW1271) induced robust proliferation of both CD4+ helper TCR-T cells and CD4 cytotoxic TCR-T cells across all three batches of TSC-204-A0201 TCR-T cells (FIGS. 11A-11C, TSC-204-A0201 panels). Cytotoxic TCR-T cells proliferated somewhat better than helper TCR-T cells, both from a point of view of the total percentage of proliferating T cells and the percentage of T cells that underwent 3 or more cell cycles (FIGS. 11B and 11C).

In contrast to the MAGE-A1-positive and HLA-A*02:01-positive cancer cell lines, A101D, which expresses MAGE-A1 but is negative for HLA-A*02:01, induced little to no proliferation of TSC-204-A0201 TCR-T cells, demonstrating the specificity of the proliferative responses (FIG. 11E).

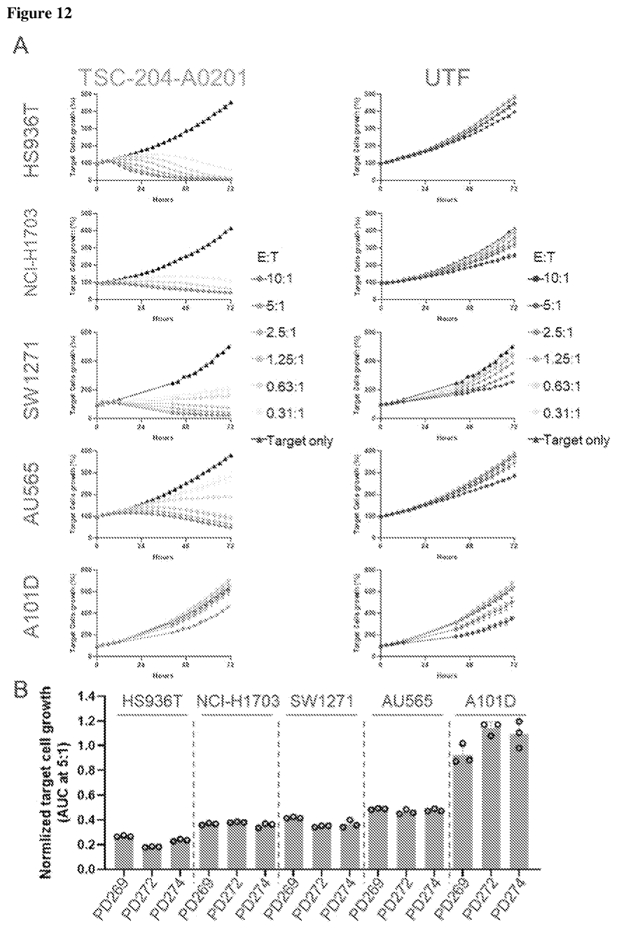

As an additional control, proliferative responses of UTF control T cells generated from matched donors were also assessed. UTF control T cells exhibited high base line proliferation in the absence of cancer cell lines. The observed baseline proliferation was especially high for UTF from PD274: ~40% of UTF helper T cells and 60% of UTF cytotoxic T cells proliferated (see dashed lines for UTF in FIG. 11). Co-culture with HS936T and AU565 appeared to suppress baseline proliferation of UTF control T cells (see dashed lines for UTF in FIGS. 11A and 11D). The suppression of T cell proliferation by these cancer cell lines could be caused by nutrient depletion, or, alternatively, indicate the engagement of immuno-suppressive pathways (for instance, PDL1-PD1 pathway). On the other hand, three of the cancer cell lines (NCIH1703, SW1271 and A101D) appeared to stimulate the baseline proliferation of UTF control T cells: the percentage of proliferating T cells was increased up to 2-fold compared to the baseline (FIGS. 11B-11D). Since co-culture with these cancer cell lines did not stimulate cytokine production or elicit cytotoxic responses from UTF control T cells (FIGS. 11 and 12), it is believed that the observed proliferation was driven by weak interactions of the endogenous TCR of T cells with these targets. When TSC-204-A0201 TCR T cells were co-cultured with cell lines that also stimulate the proliferation in UTF T cells (i.e., NCIH1703 and SW1271), the fold-change from baseline to induced proliferation of TSC-204-A0201 TCR-T cells far exceeded the increase observed in UTF control T cells (6-9-fold versus 2-fold), indicating that the proliferation in TSC-204-A0201 TCR T cells was driven by the target-dependent engagement of the TCR-T cells.

Taken together, these data show that TSC-204-A0201 TCR-T cells are capable of mounting a robust proliferative response when encountering target cells that express MAGE-A1 and HLA-A*02:01. This proliferative response of TSC-204-A0201 TCR-T cells appeared to be target-dependent and highly specific, since little or no proliferation was observed in response to target cells that express MAGE-A1 but lack HLA-A*02:01 expression (A101D), or have very low HLA-A*02:01 expression (i.e., AU565). Both the helper and cytotoxic T cells making up the TSC-204-A0201 TCR-T cells were functionally engaged.

e. TSC-204-A0201 TCR-T Cells Exhibit Selective and Potent Cytotoxic Function

assess the cytotoxicity potential of TSC-204-A0201 TCR-T cells, three independent batches of process-representative TSC-204-A0201 TCR-T cells were tested in an IncuCyte-based cytotoxicity assay. The effector T cells were serially diluted and co-cultured with a fixed number of cancer cell lines to test different effector to target ratios (E:T). Untransfected (UTF) control T cells from matching donors were similarly tested as negative controls. The target cell lines tested corresponded to the panel also used to test induction of cytokine secretion and proliferation and comprised MAGE-A1-positive, HLA-A*02:01-positive targets (i.e., NCI-H1703, SW1271, AU565 and HS936T) as well as a negative control target cell line (i.e., A101D, which is negative for HLA-A*02:01). These cells were engineered to express NuclightRed, a fluorescent protein enabling the tracking and quantification of cell growth over time.

Speficially, targets were engineered to express the fluorescent protein NuclightRed (IncuCyte® NucLightRed, Essen Bioscience) following the manufacturer's instruction to allow tracking of target cells in the IncuCyte®-based cytotoxicity assay. Target cells were plated the day before initiating the co-culture as described above. Effectors were thawed and rested overnight as described above. Serial dilutions of effectors were prepared in cytokine-free T cell medium to obtain the plating concentrations and 100 μL of effectors were added to the targets, resulting in an E:T titration ranging from 10:1 to 0.3:1. For the target cell only condition 100 μL of cytokine-free T cell medium was added to the target cells. Plates were sealed with breathable plate sealer to limit evaporation of medium and were allowed to settle at room temperature for 10-15 minutes. After an additional 15 minutes incubation at 37° C. 5% CO₂, a kimwipe was used to wipe off condensation of the bottom of the plates and acquisition was started. Data acquisition and image analysis were performed on Sartorius IncuCyte equipment and software. The raw data were exported and graphed in GraphPad Prism (v5.02).

The data for TSC-204-A0201 TCR-T cells and UTF T cells from donor PD272, which are representative of the batches tested, are presented in FIG. 12A.

The UTF T cells displayed no cytotoxicity against any of the target cells tested (FIG. 12A). On the other hand, the TSC-204-A0201 TCR-T cells displayed a potent and selective cytotoxic function. TSC-204-A0201 TCR-T cells produced a dose-dependent killing response leading to a contraction of the target cells at high E:T or the control of their growth at low E:T upon co-culture with cell lines naturally presenting the targeted MAGE-A1 epitope on HLA-A*02:01 (i.e., HS936T, NCI-H1703, SW1271 and AU565). HLA-A*02:01-negative target cell line A101D did not elicit a cytotoxic response from any of the batches of TSC-204-A0201 TCR-T cells (FIG. 12A).

The growth rate (i.e., area under the curve of target cell growth over 72 hour) of the target cell lines co-cultured with the TSC-204-A0201 TCR-T cells at an E:T of 5:1, normalized to the growth rate of the target cell lines observed upon co-culture with the corresponding UTF control T cells for all 3 batches is presented in FIG. 12B. The magnitude of the cytotoxic response against each target was comparable across the batches with virtually no donor-to-donor variability and all batches tested displayed a potent cytotoxicity activity toward the cell lines naturally presenting the targeted epitope on HLA-A*02:01 (i.e., HS936T, NCI-H1703, SW1271 and AU565). This cytotoxic response appeared selective as none of the batches of TSC-204-A0201 TCR-T cells impaired the growth of the HLA-*A02:01-negative cell line, A101D.

e. TSC-204-A0201 TCR-T Cells Exhibit Resistance to TGFbeta Signaling

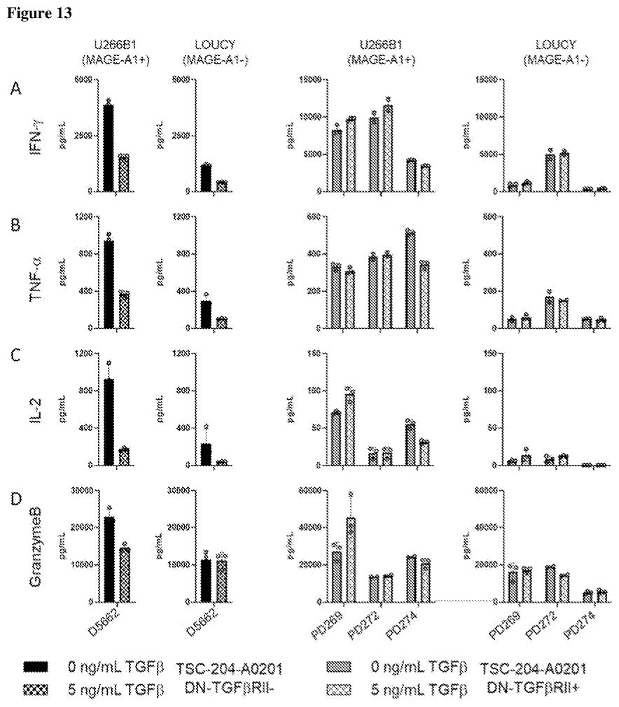

FIG. 13 show the ability of the engineered TSC-204-A0201 TCR-T cells to resist TGFβ-mediated inhibition of T cell function. Briefly, TSC-204-A0201 TCR-T cells were exposed to TGFβ and co-cultured with target cells. The ability of the TCR-T cells to respond to their cognate target by inducing the secretion of granzyme B, as well as secretion of the pro-inflammatory cytokines IFN-γ IL-2 and TNF-α, in the presence of TGFβ was tested and confirmed.

Briefly, the TCR-T cells tested were pre-incubated for ~20 h with TGFβ (0 or 5 ng/mL) prior to being incubated for 20 hours with U266B1 target cells (MAGE-A1-positive, HLA-A*02:01-positive cells). At this point, the T cells were spun down, supernatant was completely removed, and a second round of target cells was added. TGFβ was maintained at a concentration of 0 or 5 ng/mL throughout the two rounds of co-culture. Cytokines (IFN-γ, TNF-α, and IL-2), as well as granzyme B secretion, were evaluated after the second round of co-culture. Besides U266B1, a negative control cell line, i.e., HLA-A*02:01-positive MAGE-A1-negative LOUCY cells, was also used in the second round of co-culture. This condition was included to measure the amount of cytokine and granzyme B produced at the end of the experiment that derived from the first round of stimulation with U266B1.

The three independent batches of process-representative TSC-204-A0201 TCR-T cells were tested, along with a control T cell material consisting of process-similar TSC-204-A0201 material lacking DN-TGFβRII. This material was produced for the study described in Example 15 below and was used here to control for proper TGFβ-induced immunosuppression.

In more detail, serial co-culture assay was conducted to evaluate resistance to TGFβ-mediated inhibition of cytokine and granzyme B secretion. Effectors were thawed and allowed to recover overnight as described above. Subsequently, 5E4 live effectors per well were plated in 100 μL T cell medium in a 96 well round bottom plate and 100 μL of 0 or 10 ng/mL TGFβ1 diluted in T cell medium was added for a final concentration of 0 or 5 ng/ml TGFB1. After 20-24 hour incubation at 37° C., 5% CO₂, effectors were spun down and the supernatant was carefully discarded without disturbing the cell pellets. Effectors were resuspended in 100 μL of 0 or 10 ng/mL TGFB1 diluted in T cell medium, and 5E4 live U266B1 in 100 μL target cell medium was added for a final TGFβ concentration of 0 or 5 ng/ml and an E:T of 1:1. After 20 hour co-culture, effectors were spun down, supernatant was discarded and effectors were resuspended in T cell medium+/−TGFβ1 as described above. A second round of targets, either 5E4 live U266B1 or 5E4 live Loucy in 100 μL target cell medium was added, and effectors were co-cultured with targets for an additional 20 hours. Subsequently, plates were spun down and supernatants were collected for evaluation of cytokine and Granzyme B secretion using an automated ELISA system (ELLA by ProteinSimple) in conjunction with 4-plex (IFN-γ, TNF-α, IL-2 and Granzyme B) ELLA cartridges. The raw data were exported as excel files and graphed in GraphPad Prism (v5.02).

In the absence of TGFβ, both control TCR-T cells lacking DN-TGFβRII (black bars (i.e., left two columns), FIG. 13) and all three batches of process-representative TCR-T cells expressing DN-TGFβRII (orange bars (i.e., right two columns), FIG. 13) displayed a robust target-dependent production of cytokine and of Granzyme B, with induction of secretion observed upon co-culture with U266B1co-cultured with LOUCY cells (FIG. 13). This later observation confirmed that most of the cytokine and of granzyme B measured after two rounds of co-culture with U266B1 derived from the second round of stimulation.

In the presence of TGFβ (5 ng/ml), control TSC-204-A0201 TCR-T cells lacking DN-TGFβRII showed a 2-3-fold reduction in the total secreted amount of all three cytokines (IFN-γ, TNF-α, and IL-2) and a reduction of granzyme B secretion (black bars (i.e., left two columns), FIG. 13A). TGFβ represses the transcription of genes needed for T cell effector function, including IFN-γ, IL-2 and granzyme B. However, since a decrease in T cell numbers when TSC-204-A0201 TCR-T cells lacking DN-TGFβRII were co-cultured with U266B1 in the presence of TGFβ was also observed (see data presented in Example 15 below), it is believed that the TGFβ mediated reduction in cytokine and granzyme B secretion was due to a combination of transcriptional repression of those genes, and of reduced T cell survival in the presence of TGFβ.

In contrast to DN-TGFβRII-negative control TCR-T cells, process-representative TSC-204-A0201 TCR-T cells (which express DN-TGFβRII) displayed little to no reduction of cytokine and granzyme B secretion (orange bars (i.e., right two columns), FIG. 13B). This observation supports that expression of the DN-TGFβRII shields the TCR-T cells from TGFβ-mediated inhibition of cytokine production. TGFβ inhibited Granzyme B secretion of DN-TGFβRII-negative control TCR-T cells (see Example 15 below) with variation attributed to variable depletion of pre-formed granzyme B protein. Taken together with the proliferation data presented in Example 15 below, these data confirm that expression of the DN-TGFβRII confers resistance to TGFβ-mediated inhibition of T cell function of process-representative TCR-T cells.

### Example 11: In Vivo Efficacy of TSC-204-A0201

In vivo the anti-tumor activity, tolerability, and persistence of a representative number of independent donor-derived batches of TSC-204-A0201 TCR T cells prepared as described in Example 10 above and administered intravenously in a representative cancer model (xenograft model of U266B1 in female NCG mice) were evaluated. TSC-204-A0201 TCR-T cells displayed a strong and potent anti-tumor activity (tumor growth inhibition, TGI) with both batches. Based on body weight and clinical observations all the treatment groups were well tolerated. No overt signs of toxicity were observed and there were no apparent treatment-related deaths. TSC-204-A0201 TCR-T-cells were detected in circulation in the blood of the animals. Repeat dosing appeared to favor the persistence of the therapeutic T cells that were still detected for both batches of TSC-204-A0201 tested, up to day 21 after the first dose (14 days after the second dose).

In particular, efficacy of the two batches of TSC-204-A0201 from different donors was compared to non-engineered (untransfected [UTF]) control T cells from matching donors, and vehicle (PBS) treatments against the U266B1 tumor cell line implanted in NCG female mice. U266B1 cell line is derived from a B lymphocyte myeloma. These tumor cells endogenously express MAGE-A1 and HLA-A*02:01 and were confirmed to be recognized by TSC-204-A0201 TCR-T cells in vitro (see Example 10 above). Tumor cells were inoculated subcutaneously in the right flank of the animals. Once tumor engraftment was successful (tumors reaching 100 mm³ on average), animals were randomized into different treatment groups, and received two doses, 7 days apart, of TSC-204-A0201, of UTF control T cells, or of vehicle (PBS). Different readouts were gathered over time: (1) anti-tumor efficacy was evaluated by biweekly tumor volume measurements; (2) proportion of circulating human T cells was assessed by flow cytometry in blood samples collected on day 2, 8, 14, and 21 post-initiation of TSC-204-A0201 TCR T cells treatment; (3) biweekly body weight measurements were recorded alongside any clinical observations to gauge any toxicity related to TSC-204-A0201 injection.

Materials and methods used for in vivo efficacy tests are described herein. Briefly, the U266B1 cell line, derived from a B lymphocyte myeloma was purchased from ATCC and cultured according to the manufacturer's recommendations. The cell culture medium used to grow this cell line was RPMI-1640 (ATCC) supplemented with 15% FBS (Gibco). The tumor cells were maintained at log phase growth in tissue culture flasks in a humidified incubator at 37° C., in an atmosphere of 5% CO₂ and 95% air.

On the days of dosing, cryovials (5 ml) of cryopreserved TSC-204-A0201 TCR-T cells and UTF control T cells from matching donors (each having comparable amounts of viable cells) were thawed (one group at a time) using 37° C. bath and transferred in thawing media consisting of X-VIVO™ 15 media (Lonza Cat #04-418Q), and 5% heat-inactivated human serum (Sigma Cat No. H3667), resuspended and rinsed in sterile PBS to wash the cells and resuspended in PBS to obtain dosing solutions at concentrations of 2E7 viable CD34⁺ cells per 0.2 mL. The total amount of T cell injected was adjusted to account for CD34⁺ purity.

Two hundred (200 female) CR mice NOD-Prkdc^{em26Cd52}Il2r^{gem26Cd22}/NjuCrl (NCG) mice were ordered and inoculated with tumor cells for potential assignment to the study. Mice were 9 weeks old, with body weights ranging from 18.9 to 27.5 g at the beginning of the study.

U266B1 cells used for subcutaneous xenografts were harvested during log phase growth and washed in PBS. Mice were inoculated subcutaneously with 100 μL of a mix of 80% Matrigel/20% medium containing 10E7 live U266B1. Tumors were measured by caliper biweekly starting one week post inoculation and continuing until the end of the study. Tumor measurements were recorded in Overwatch software. When average tumor volumes reached 100 mm³ (21 days post inoculation, tumor volumes ranged from 36.9 mm³ to 110.1 mm³), animals were randomized to produce 5 groups for the study of anti-tumor efficacy and 2 groups for the T cell persistence study.

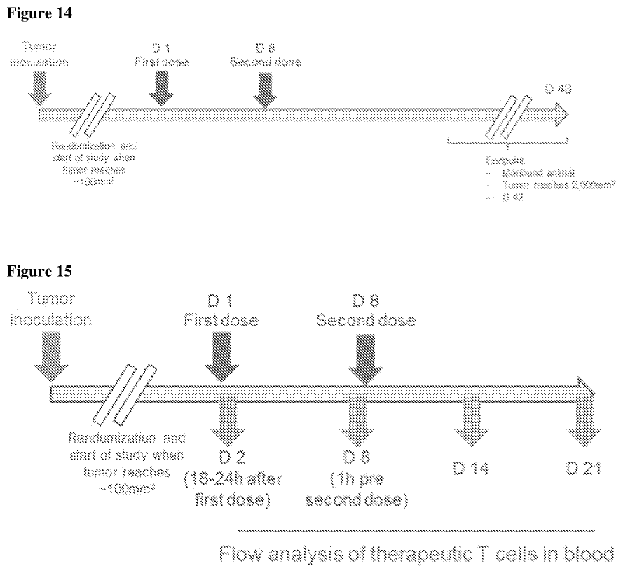

Animals received repeat doses on day 1 and day 8 according to the study design presented in Error! Reference source not found., FIG. 14, and FIG. 15. The dosing volume was 200 μL, administered intravenously (I.V.). Group 1 received injections of vehicle control (PBS); group 2 received injections untransfected (UTF) control T cells from batch PD269 (2.9E7 total T cells); group 3 and group 6 received injections of TSC-204-A0201 from batch PD269 (2E7 CD34⁺, corresponding to 2.8E7 total T cells), group 5 and group 7 received injections of TSC-204-A0201 from batch PD272 (2E7 CD34⁺, corresponding to 2.4E7 total T cells).

TABLE 10
Experimental design and reagents
Experimental design

| | | | Formulation | Formulation | | | |
|---|---|---|---|---|---|---|---|
| | | | dose | dose (Total | | | |
| GR. | N | Agent | (CD34+) | T cells) | Route | Schedule | Purpose |
| 1 | 12 | Vehicle | NA | NA | iv | days 1, 8 | Efficacy |
| 2 | 12 | Donor #1- UTF | NA | 2.91E7 | iv | days 1, 8 | |
| 3 | 12 | Donor #1- TSC- | 2E7 | 2.87E7 | iv | days 1, 8 | |
| | | 204-A0201 | | | | | |
| 4 | 12 | Donor #2- UTF | NA | 2.52E7 | iv | days 1, 8 | |
| 5 | 12 | Donor #2- TSC- | 2E7 | 2.4E7 | iv | days 1, 8 | |
| | | 204-A0201 | | | | | |
| 6 | 7 | Donor #1- TSC- | 2E7 | 2.87E7 | iv | days 1, 8 | T cell |
| | | 204-A0201 | | | | | persistence: |
| 7 | 7 | Donor #2- TSC- | 2E7 | 2.4E7 | iv | days 1, 8 | |
| | | 204-A0201 | | | | | |

Note: Donor #1: batch PD269; Donor #2: batch PD272.
Reagents

| Media | Vendor | Catalog Number |
|---|---|---|
| (RPMI) 1640 Medium | Gibco | 30-2001 |
| Fetal Bovine Serum (Heat-Inactivated) | Gibco | A38400 |
| DPBS (Dulbecco's phosphate-buffered saline) | Gibco | 14190-136 |
| Reagent | Vendor | Catalog Number |
| 0.4% Quatricide | Medline | N/A (From Explora) |
| ACK | Gibco | A1049201 |
| Wescodyne | West Penetone | 1511 |
| DPBS | Gibco | 14190-136 |
| EasySep Buffer | StemCell | 20144 |
| Fc block (TruStain FcX) | BioLegend | 422302 |
| FBS | Gibco | 35050-001 |
| DMSO | Sigma | D265-100 ml |
| GlutaMax (100x) | Gibco | 35050-061 |
| RPMI | ATCC | 30-2001 |
| ViaStain AOPI Staining Solution | Nexcelom Bioscience | CS2-0106-25 mL |
| BD Cytofix ™ Fixation Buffer | BD BioScience | 554655 |
| 70% Isopropanol | Decon Laboratories Inc | 8601 |
| BD Cytofix | BD | 554655 |

Throughout the study, mice were observed for general health/mortality and moribundity twice daily, once in the morning and once in the afternoon. Cage-side observations occurred daily. Mice were not removed from their cage during observation, unless necessary for identification or confirmation of possible findings. The mice were also observed for overt signs of any adverse treatment-related (TR) side effects, during sampling, body weight and tumor measurement activities. Body weights were recorded on days 0, 3, 6, 9, 12, 15, 19, 22, 25, 28, 33, 36, 40, and 43 of the study. Tumors were measured by caliper (mm units) biweekly until day 43 of the study and recorder in the Overwatch software. The greatest longitudinal diameter (length [L]) and the greatest transverse diameter (width [W]) were determined and reported into the Overwatch software. The tumor volume was estimated using the ellipsoidal formula V=(W²×L)/2.

The differences in the tumor growth over time between pairs of treatment groups were assessed by fitting each animal's data to a simple exponential growth model and comparing the mean growth rates of two groups. The difference in the growth rates was summarized by the growth rate inhibition (GRI), which is the reduction in the growth rate experienced by the treatment group relative to that of the Control-treated group, expressed as a fraction of the vehicle growth rate. A positive GRI indicates that the tumors in the treatment group grew at a reduced rate relative to the reference group. GRI was calculated on day 21 post-treatment when all groups displayed 100% of live animals. The tumor volumes were log transformed, and the growth rate for each animal was calculated as the slope of the log volume vs. time.

GRI=100%×(mean growth rate for control−mean growth rate for treatment)/mean growth rate for control.

The endpoint of the experiment was death or moribundity due to tumor progression, tumor volume reaching 2,000 mm³, or the last day of the study (day 43 post-initiation of treatments, corresponding to day 64 post-inoculation of tumor cells).

Animals were monitored individually for signs of moribundity, decreases in body weight and tumor progression and classified as death on study. The time to endpoint (TTE), in days, was recorded for each mouse that died of its disease or was euthanized due to tumor progression. Any animal classified as having died from treatment-related (TR) causes was assigned a TTE value equal to the day of death. Any animal that did not appear moribund but was euthanized due disease progression as supported by necropsy observations, was recorded as a non-treatment-related death due to tumor invasion or metastasis (NTRm) and was included in the data analysis. Any death due to unknown causes (NTRu) or due to an accident or error (NTRa) was excluded from TTE calculations and all further analyses.

In T cell persistence evaluation assays, animals in groups 6 and 7 were used to evaluate the concentration of circulating TSC-204-A0201 TCR-T cells over time, in animals receiving two doses of test article. Blood samples were collected by submandibular bleeding without anesthesia from all animals in groups 6 and 7 on Days 2, 8, and 14. Cardiac puncture were performed for the terminal bleed on Day21. RBC lysis was performed on blood samples (~150 μL) using ammonium-chloride-potassium (ACK) buffer (Gibco). Samples were washed in EasySep (STEMCELL Thechnology) and filtered through a 30-40 μm 96-well filter plate (Pall Corporation) and assessed for cell count and viability. A total of 0.5-1E6 cells (using a constant cell number across samples on a given day of analysis) were transferred in a U-bottom 96-well plates and stained with the fixable viability dye near-IR according to the manufacturer's instructions. Cells were washed with EasySep buffer and resuspended in FC block stain (BioLegend) and incubated in the dark at 4° C. for 15 minutes. Antibody master mix including hCD45 antibody (Biolegend Cat #304016), mCD45 (Invitrogen, Cat #MCD45501), and hCD34 (R&D, Cat #FAB7227A) was added to each well and cells were incubated at 4° C. for 30 minutes. At the end of the incubation, cells were washed and resuspended in 200 μL of EasySep buffer for flow cytometry analysis. Unstained cells, single stain controls and FMO controls were also prepared.

Data acquisition was performed on a Cytoflex LX (acquisition volume: 150 μL, sample flow rate 60 μl/min). Compensation was performed automatically with the CytExpert software. Data analysis was performed with CytExpert software, Excel 2010 and GraphPad Prism 8.3.1.

Cells were gated from the FSC versus SSC dot plot and singlets distinguished from the aggregates using FSC-Area versus FSC-Height plot. Viable cells were identified using the near-IR Live-Dead versus SSC-Area plot. Human T cells were identified in the live blood cells based on the positive signal with the hCD45 marker. The proportion of human T cells expressing the CD34 tag was quantified to confirm that the hCD45⁺ cells correspond to TSC-204-A0201 TCR-T cells.

Mice were euthanized by asphyxiation using carbone dioxide (CO₂) followed by cervical dislocation. The time to endpoint (TTE), in days, is recorded for each mouse that dies of its disease or was euthanized due to tumor progression or study termination.

Pairwise comparisons of growth rate inhibition (GRI) were performed, where the mean GRI for the vehicle control treated animals was compared with GRI of different treatment conditions. The growth rate estimates were assumed to be normally distributed, and an unpaired t-test with unequal variances was used to check if there was a statistically significant difference between the two groups. Statistical analyses were performed using Student's t-test in GraphPad Prism® 8.0 software and P-values<0.05 were considered statistically significant. All days and all animals were included.

a. TSC-204-A0201 TCR-T Cells Exhibit In Vivo Anti-Tumor Efficacy

The efficacy of TSC-204-A0201 TCR-T cells was evaluated against the U266B1 tumor model in NCG female animals. Two batches of process-representative TSC-204-A0201 material were compared to untransfected (UTF) control T cells from matching donors, and to vehicle control (PBS) treatment. Animals with confirmed growing tumors (average tumor volumes reached 100 mm³; tumor volumes ranged from 36.9 mm³ to 110.1 mm³; 21 days post inoculation) were randomized in treatment groups and received two doses of every treatment regimen (one week apart). 2E7 CD34⁺ (i.e., engineered) TCR-T cells were injected on day 1 and day 8. Cellular dose was adjusted for the purity of the material and corresponded to 2.8E7 total T cells for PD269 batch, and 2.4E7 total T cells for the PD272 batch. UTF control T cells injected were 2.91E7, and 2.52E7 for PD269 and PD272, respectively. The number of UTF control T cells injected for each dose corresponded to the total number of T cells injected for TCR-T treatment of the matching batch.

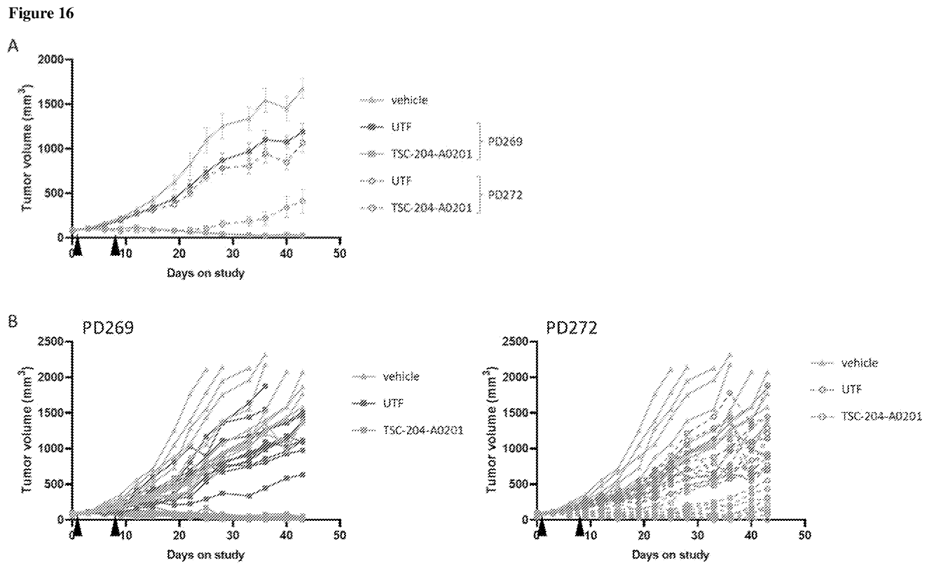

As presented in FIG. 16, animals for control groups (groups 1 [vehicle], 2, and 4 [UTF control T cells from batches PD269 and PD272, respectively]) presented large tumors at the end of the study, with mean tumor volumes on Day 43 of 1679.53 mm³, 1190.80 mm³, and 1066.46 mm³, respectively. The median TTE was 41.5 days for group 1 (vehicle), 43 days for group 2 (UTF control T cells from batch PD269), and 43 days for group 4 (UTF control T cells from batch PD272). Tumors in UTF-treated control groups (groups 2 and 4) grew at a similar rate as the vehicle-treated group (group 1), with only 29.1% and 39.1% tumor growth inhibition for groups 2 and 5, respectively (calculated on day 21 after start of treatment). There were no statistical differences when considering GRI of groups 2 or 5 over the tumor growth of group 1 (P-values of 0.4951 and 0.3684 when comparing GRI for group 2, or group 4, respectively).

The groups that received TSC-204-A0201 TCR-T cells from either batch of material (groups 3 and 5) showed a robust anti-tumor response when compared to the control groups. The median TTE for both treatment groups were 43 days (corresponding to the end of the study) which was not significantly different than the TTE observed for the vehicle control group However, both TSC-204-A0201-treated groups presented a robust tumor growth rate inhibition of 88.97% (group 3) and 82.15% (group 5) relative to their respective UTF control-treated groups (P-values of 0.0161 and 0.0136 for group 3 and group 5, respectively).

b. TSC-204-A0201 TCR-T Cells Exhibit In Vivo Delay of Mortality

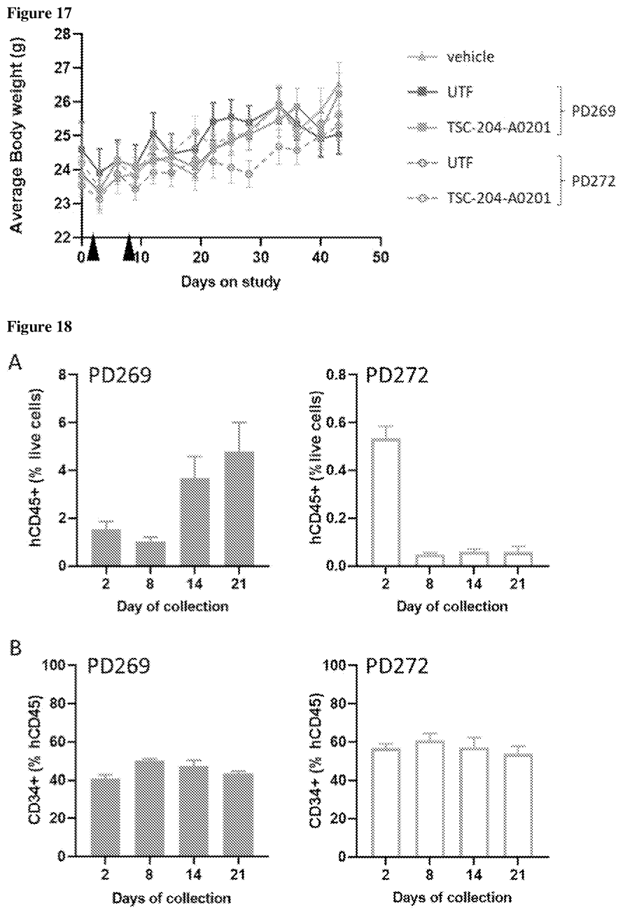

No apparent treatment-related deaths were observed. All study groups showed a trend of BW gain between day 1 up to day 43 post-treatment (FIG. 17). Bodyweight appeared consistent across groups regardless or treatment regimen, indicating no overt signs of toxicity in the treated groups.

c. TSC-204-A0201 TCR-T Cells Exhibit In Vivo Persistence

T cell persistence was also assessed. Flow cytometry analyses were conducted to measure the proportion of human immune cells in the blood of animals injected with TSC-204-A0201 from batch PD269 and PD272 on day 1 and on day 8. Blood samples were collected at different time points (FIG. 18) and analyzed by flow cytometry. Human T cells were identified in the samples based on their expression of the human CD45 marker and were distinguished from mouse cells that stained positive for mouse CD45. CD34 was also analyzed to confirm that the hCD45 cells corresponded to the TCR-T cells. TSC-204-A0201 TCR-T cells were detected 24 hour after the first injection for both batches of material (analysis on day 2; FIG. 18). TSC-204-A0201 TCR-T cells from batch PD269 displayed a 3-fold higher initial concentration of circulating human T cells on Day 2 when compared to the human T cells from batch PD272 (1.52% and 0.53% of blood cells were human T cells for PD269-treated animals and PD272-treated animals, respectively). Circulating human T cells decreased during the first week post-injection for both batches (analysis on day 8 was done prior to the second injection). Cells from PD269 dropped by ~40% (down to 1.00% of blood cells), while cells from PD272 dropped by ~90% (down to 0.047% of blood cells). The second injection of TSC-204-A0201 benefited the persistence of the TCR-T cells. When considering the batch PD269, the proportion of human T cells in the blood of the animals increased a week after the second dose (at 3.67% of blood cells on Day 14) and remained high 7 days later (reaching 4.78% of blood cells on day 21). The cell concentration for TSC-204-A0201 from batch PD272 remained stable between day 8 and day 14 and up to day 21 with human T cells remaining detectable (representing ~0.06% of blood cells).

Overall, the data indicate that repeat dosing has a positive effect on T cell persistence, leading to an increase of T cell engraftment, and long-lasting circulation.

### Example 12: Lack of TSC-204-A0201TCR-T Cell Off-Tumor Reactivity

The risk of on-target/off-tumor reactivity is low for TSC-204-A0201 TCR-T cells as MAGE-A1 is a cancer/testis protein that is produced during embryonic development but is virtually absent in all normal adult tissues except testes (Gjerstorff et al. (2007) Hum.Reprod. 22: 953-960), which is an immune privileged tissue (Li et al. (2012) Front. Immunol. 3:152; Hedger (2014) Knobil Neill Physiol. Reprod. 2015:805-892). Further, data presented in Example 14 below show that MAGE-A1 expression in normal tissue is largely restricted to testes consistent with prior literature (van der Bruggen et al. (1991) Science 254:1643-1647; Obenhaus et al. (2015) Nat. Biotechnol. 33:402-407) and publicly available RNA-sequencing data (data of MAGE-A1 mRNA expression across multiple cell types at the single cell level obtained from the Human Protein Atlas; available at world wide web at proteinatlas.org). These gene expression analyses indicate that MAGE-A1 represents a safe target for T cell-based therapies with low risk for on-target off-tumor reactivity.

The off-target reactivity of a therapeutic TCR may arise from cross-reactivity of the TCR to a self-peptide/MHC.

As described below, TSC-204-A0201 TCR-T cells were determined not to react to wide variety of primary samples lacking any significant MAGE-A1 expression, even as the cells endogenously express other putative off-target peptides such that reactivity of the TCR-T cells is expected to be restricted to cancer cells expressing MAGE-A1 and HLA-A*02:01.

Generally, TSC-204-A0201 TCR-T cells were tested for their reactivity to an extensive panel of 74 target cells comprising 4 cancer cell lines and 70 healthy human primary cells and iPSC-derived cells from multiple tissues and organs (Table 11).

TABLE 11
Target cancer cell line and primary cell descriptions
Target cancer cell line descriptions

| | | Catalog | |
|---|---|---|---|
| Cell line | Vendor | Number | Culture medium |
| U266B1 | ATCC | TIB-196 | RPMI1640 (ATCC 30-2001) + 20% FBS (Thermofisher |
| | | | Scientific A3840001) + 1X Penicillin/Streptomycin |
| | | | (Thermofisher Scientific 15149-122) |
| Loucy | ATCC | CRL-2629 | RPMI1640 (ATCC 30-2001) + 10% FBS (Thermofisher |
| | | | Scientific A3840001) + 1X Penicillin/Streptomycin |
| | | | (Thermofisher Scientific 15149-122) |
| BICR78 | ECACC | 04072111 | DMEM (Thermofisher Scientific 11965092) + 10% FBS |
| | | | (Thermofisher Scientific A3840001) + 1X GlutaMax |
| | | | (FisherScientific 35050061) + 0.4 ug/mL Hydrocortisone |
| | | | (Sigma-Aldrich H0396-100 mg) + 1X Penicillin/ |
| | | | Streptomycin (Thermofisher Scientific 15149-122) |
| MCF7 | ATCC | HTB-22 | EMEM (ATCC 30-2003) + 10% FBS (Thermofisher |
| | | | Scientific A3840001) + 0.01 mg/mL bovine |
| | | | insulin (Sigma-Aldrich I0516-5ML) |
| HEK293T | ATCC | CRL-3216 | DMEM (Thermofisher Scientific 11965092) + 10% FBS |
| | | | (Thermofisher Scientific A3840001) + 1X |
| | | | Penicillin/Streptomycin (Thermofisher |
| | | | Scientific 15149-122) |

Target primary and iPSC-derived cell descriptions

| Primary/iPSC- | Lot/batch | Catalog | | |
|---|---|---|---|---|
| derived cell type | number | number | Vendor | Culture Medium (catalog number) |
| Peripheral Blood | 12429 | | AllCells | X-VIVO15 (Lonza 04-418Q) + 5% |
| Mononuclear | | | | Human serum (Sigma-Aldrich |
| Cells (PBMC) | 888607014 | | DLS | H3667-100) + 1X GlutaMax |
| | 110044327 | | DLS | (FisherScientific #35050061) + 1X |
| | | | | Penicillin/Streptomycin |
| | | | | (Thermofisher Scientific 15149-122) |
| Human | 474Z035 | A-12991 | PromoCell | Endothelial Cell Growth Medium 2 |
| Umbilical Vein | 474Z036 | C-12206 | | (C-22011) |
| Endothelial Cells | 466Z026 | C-12200 | | Endothelial growth medium (C- |
| (HUVEC) | | | | 22010) |
| Normal Human | 483Z001.1 | C-12001 | PromoCell | Keratinocyte Growth Medium 2 (C- |
| Epidermal | 425Z026.2 | C-12003 | | 20011) |
| Keratinocytes | 451Z014.1 | C-12003 | | |
| (NHEK) | | | | |
| Normal Human | 471Z018.2 | C-12300 | PromoCell | Fibroblast Growth Medium (C- |
| Dermal | | | | 23010) |
| Fibroblasts | 472Z001.3 | C-12302 | | Fibroblast Growth Medium 2 (C- |
| (NHDF) | 477Z023.2 | C-12302 | | 23020) |
| Human | 1081503.2 | C-12360 | PromoCell | Fibroblast Growth Medium 2 (C- |
| Pulmonary | 474Z031.2 | C-12360 | | 23020) |
| Fibroblasts | 474Z024.2 | C-12360 | | |
| (HPF) | | | | |
| Human Cardiac | 475Z017.1 | C-12375 | PromoCell | Fibroblast Growth Medium 3 (C- |
| Fibroblasts | 472Z002.2 | C-12375 | | 23025) |
| (HCF) | 470Z011.10 | A-12992 | | |
| Human Aortic | 437Z016.2 | C-12533 | PromoCell | Smooth Muscle Cell Growth |
| Smooth Muscle | 453Z011.6 | C-12533 | | Medium 2 (C-22062) |
| Cells (HAoSMC) | | | | |
| Human Small | 467Z033 | C-12642 | PromoCell | Small Airway Epithelial Cell |
| Airway | 467Z025.2 | C-12642 | | Growth Medium (C-21070) |
| Epithelial Cells | 20TL119096 | CC-2547 | Lonza | SAGM ™ Small Airway Epithelial |
| (HSAEpC) | | | | Cell Growth Medium BulletKit ™ |
| | | | | (CC-3118) |
| Human | 0000613768 | CC-2576 | Lonza | SmGM ™-2 Smooth Muscle Cell |
| Bronchial | 21TL104462 | CC-2576 | | Growth Medium-2 BulletKit ™ |
| Smooth Muscle | | | | (CC-3182) |
| Cells (HBSMC) | | | | |
| Human | 20TL119094 | CC-2540 | Lonza | BEGM ™ Bronchial Epithelial Cell |
| Bronchial | | | | Growth Medium BulletKit ™ (CC- |
| Epithelial Cells | | | | 3170) |
| (HBEpC) | 469Z016 | C-12640 | PromoCell | Airway Epithelial Cell growth |
| | 482Z007.15 | C-12640 | | Medium (C-21060) |
| Normal Human | 0000474577 | CC-2586 | Lonza | MGM ™-4 Melanocyte Growth |
| Epidermal | 0000495833 | CC-2504 | | Medium-4 BulletKit ™ (CC-3249) |
| Melanocytes | 0000488138 | CC-2586 | | |
| (NHEM) | | | | |
| Human Small | 201457 | 10HU- | iXCells | Epithelial Cell Growth Medium |
| Intestinal | | 237 | Biotech | (Cat# MD-0041) |
| Epithelial Cells | 202152 | 10HU- | | |
| (HSIEpC) | | 237 | | |
| | FF14160314/17 | H-6051 | Cell Biologics | Complete Epithelial Cell Medium /w |
| | | | | Kit-500 ML (M-6621) |
| Human Cervical | 70029809 | PCS-480- | ATCC | Cervical Epithelial Cell Basal |
| Epithelial Cells | | 011 | | Medium (PCS-480-032) + Cervical |
| (HCerEpC) | | | | Epithelial Cell Growth Kit (PCS- |
| | | | | 480-042) |
| | 06226 | FC-0080 | Lifeline Cell | ReproLife ™ CX Basal Medium |
| | | | Technology | (LM-0055) + ReproLife CX |
| | | | | LifeFactors Kit (LS-1116) |
| Human Cardiac | 463Z016.2 | C-12810 | PromoCell | Myocyte Growth Medium (C-22070) |
| Myocytes | 470Z011.9 | C-12810 | | |
| (HCM) | 472Z002.3 | C-12810 | | |
| Human White | 465Z008.3 | C-12735 | PromoCell | Preadipocyte Growth Medium (C- |
| Preadipocytes- | 454Z006 | C-12735 | | 27410) |
| subcutaneous | 467Z014 | C-12735 | | Preadipocyte Differentiation |
| (HWP) | | | | Medium (C-27436) |
| | | | | Adipocyte Nutrition Medium (C- |
| | | | | 27438) |
| iCell ® | 105993 | C1037 | Fujifilm | DMEM/F-12, HEPES |
| Astrocytes, | | | Cellular | (ThermoFisher 11330) + FBS + N-2 |
| 01434 | | | Dynamics | supplement (ThermoFisher |
| | | | | 17502048) |
| Astrocytes | 0000672445 | CC-2565 | Lonza | AGM ™ Astrocyte Growth Medium |
| | | | | BulletKitT ™ (CC-3186) |
| Hepatocytes | HH1052 | 82006 | In Vitro | UCRM (81015) and UPCM (81016) |
| | HH1165 | 82006 | ADMET | |
| | HH1050 | 82006 | Laboratories | |
| Human Hepatic | C212011 | CSC- | Creative | SuperCult ® Stellate Cell Medium |
| Stellate Cells | | C1496 | | (SM-1193W) |
| (HHSteC) | C212641 | CSC- | Bioarray | |
| | | C1496 | | |
| | 25914 | 5300 | ScienCell | Stellate Cell Medium (5301) |
| Gastric Epithelial | F052416.19 | H-6039 | Cell Biologics | Complete Epithelial Cell Medium/w |
| Cells (GEC) | | | | Kit-500 ML (M-6621) |
| | 2361 | ABC- | Accegen | Gastric Epithelial Cell Media |
| | | HP021X | | (ABM-M021X) |
| Human Skeletal | 4820 | 3500 | ScienCell | Skeletal Muscle Cell Medium |
| Muscle Cells | 25571 | 3500 | | (SkMCM, 3501) |
| (HSKMC) | 81202212 | PCS-950- | ATCC | Mesenchymal Stem Cell Basal |
| | | 010 | | Medium for Adipose, Umbilical and |
| | | | | Bone Marrow-derived MSCs (PCS- |
| | | | | 500-030) + Primary Skeletal Muscle |
| | | | | Growth Kit (PCS-950-040) |
| Human Renal | F051616Y59 | H-6034 | Cell Biologics | Complete Epithelial Cell Medium /w |
| Epithelial Cells | | | | Kit-500 ML (M-6621) |
| (HREpC) | | | | |
| Retinal Pigment | 23522 | 6540 | ScienCell | Epithelial Cell Medium (4101) |
| Epithelial Cells | 20876 | 6540 | | |
| (RPEC) | | | | |
| Human Prostate | 06335 | FC-0038 | Lifeline Cell | ProstaLife basal medium (LM-0017) + |
| Epithelial Cells | 02321 | FC-0038 | Technology | ProstaLife LifeFactors kit (LS- |
| (HPrEpC) | | | | 1072) |
| Human Bladder | 05972 | FC-0079 | Lifeline Cell | UroLife ™ Basal Medium (LM- |
| Epithelial Cells | | | Technology | 0054) + UroLife LifeFactors (LS- |
| (HB1EpC) | | | | 1115) |
| | F051616 | H-6215 | Cell Biologics | Complete Epithelial Cell Medium /w |
| | | | | Kit-500 ML (M-6621) |
| Human | MM14160603 | H-6037 | Cell Biologics | Complete Epithelial Cell Medium /w |
| Pancreatic | | | | Kit-500 ML (M-6621) |
| Epithelial Cells | | | | |
| (HPanEpC) | | | | |
| Human | F-1416 | H-6046 | Cell Biologics | Complete Epithelial Cell Medium /w |
| Esophageal | | | | Kit-500 ML (M-6621) |
| Epithelial Cells | | | | |
| (HEsoEpC) | | | | |
| Human | 70043304 | PCS-600- | ATCC | Mammary Epithelial Cell Basal |
| Mammary | | 010 | | Medium (ATCC PCS-600-030) + |
| Epithelial Cells | | | | Mammary Epithelial Cell Growth |
| (HMEpC) | | | | Kit (ATCC PCS-600-040) |
| | 03025 | FC-0063 | Lifeline Cell | MammaryLife ™ Basal Medium |
| | | | Technology | (LM-0041) + MammaryLife |
| | 02904 | FC-0065 | Lifeline Cell | LifeFactors Kit (LS-1088) |
| | | | Technology | |
| Human Uterine | 452Z019 | C-12575 | PromoCell | Smooth Muscle Cell Growth |
| Smooth Muscle | | | | Medium 2 (C-22062) |
| Cells (HUtSMC) | 02614 | FC-0075 | Lifeline Cell | VascuLife ® Basal Medium (LM- |
| | | | Technology | 0002) + VascuLife SMC LifeFactors |
| | | | | (LS-1040) |
| iCell ® | 105979 | C1012 | Fujifilm | iCell Neural Base Medium 1 |
| GABANeurons, | | | Cellular | (M1010) + iCell Neural Supplement |
| 01434 | | | Dynamics | A (M1032) |

Text in bold in the Lot/batch number column was used to identify the lot/batch of the target
cells in the graphs. PBMCs were isolated from whole Leukopaks purchased from the vendors
indicated in the table.
T cell media

| Media | Components | Stock (mL) | Catalog Number |
|---|---|---|---|
| “Cytokine-free T cell medium” | X-VIVO 15 | 1000 | Lonza#04-418Q |
| | Human Serum (Heat- | 50 | Sigma-Aldrich#H3667-100 |
| | Inactivated) | | |
| | Pen Strep | 10 | Gibco#15149-122 |
| | GlutaMAX (100x) | 10 | FisherScientific#35050061 |
| “T cell medium with Cytokines” | Cytokine-free T cell | 300 | NA |
| | medium | | |
| | Human IL-2 (5 μg/mL) | 1.5 | Sigma-Aldrich#11147528001 |
| | Human IL-7 (10 μg/mL) | 0.150 | R&D Systems#207-IL-025 |

Media and supplements

| Media | Vendor | Catalog Number |
|---|---|---|
| X-VIVO 15 | Lonza | 04-418Q |
| Human Serum (Heat-Inactivated) | Sigma-Aldrich | H3667-100 |
| FBS, heat-inactivated | Thermofisher Scientific | A3840001 |
| GlutaMAX (100x) | Fisher Scientific | 35050061 |
| Penicillin-Streptomycin | Gibco | 15140-122 |
| RPMI medium 1640 | Gibco | A10491-01 |
| DMEM | Gibco | 11965 |
| EMEM medium | ATCC | 30-2003 |
| Insulin solution from bovine pancreas | Sigma-Aldrich | I0516 |
| Hydrocortisone-water soluble | Sigma-Aldrich | H0396-100mg |
| Endothelial Cell Growth Medium 2 | PromoCell | C-22011 |
| Endothelial Cell Growth Medium | PromoCell | C-22010 |
| Keratinocyte Growth Medium 2 | PromoCell | C-20011 |
| Fibroblast Growth Medium | PromoCell | C-23010 |
| Fibroblast Growth Medium 2 | PromoCell | C-23020 |
| Fibroblast Growth Medium 3 | PromoCell | C-23025 |
| Smooth Muscle Cell Growth Medium 2 | PromoCell | C-22062 |
| Small Airway Epithelial Cell Growth Medium | PromoCell | C-21070 |
| SAGM ™ Small Airway Epithelial Cell Growth Medium | Lonza | CC-3118 |
| BulletKit ™ | | |
| SmGM ™-2 Smooth Muscle Cell Growth Medium-2 | Lonza | CC-3182 |
| BulletKit ™ | | |
| BEGM ™ Bronchial Epithelial Cell Growth Medium | Lonza | CC-3170 |
| BulletKit ™ | | |
| Airway Epithelial Cell growth Medium | PromoCell | C-21060 |
| MGM ™-4 Melanocyte Growth Medium-4 BulletKit ™ | Lonza | CC-3249 |
| Epithelial Cell Growth Medium | iXCells Biotechnology | MD-0041 |
| Complete Epithelial Cell Medium /w Kit-500 ML | Cell Biologics | M-6621 |
| Cervical Epithelial Cell Basal Medium | ATCC | PCS-480-032 |
| Cervical Epithelial Cell Growth Kit | ATCC | PCS-480-042 |
| ReproLife ™ CX Basal Medium | Lifeline Cell Technology | LM-0055 |
| ReproLife CX LifeFactors Kit | Lifeline Cell Technology | LS-1116 |
| Myocyte Growth Medium | PromoCell | C-22070 |
| Preadipocyte Growth Medium | PromoCell | C-27410 |
| Preadipocyte Differentiation Medium | PromoCell | C-27436 |
| Adipocyte Nutrition Medium | PromoCell | C-27438 |
| DMEM/F-12, HEPES | ThermoFisher | 11330 |
| N-2 supplement | Thermo Fisher | 17502048 |
| AGMT ™ Astrocyte Growth Medium BulletKit ™ | Lonza | CC-3186 |
| Universal Cryopreserved Hepatocyte Recovery Medium | In Vitro ADMET | 81015 |
| (UCRM) | Laboratories | |
| Universal Hepatocyte Primary Cell Plating Medium | In Vitro ADMET | 81016 |
| (UPCM) | Laboratories | |
| SuperCult ® Stellate Cell Medium | Creative Bioarray | SM-1193W |
| Stellate Cell Medium | ScienCell | 5301 |
| Gastric Epithelial Cell Media | Accegen | ABM-M021X |
| Skeletal Muscle Cell Medium | ScienCell | 3501 |
| Mesenchymal Stem Cell Basal Medium for Adipose, | ATCC | PCS-500-030 |
| Umbilical and Bone Marrow-derived MSCs | | |
| Primary Skeletal Muscle Growth Kit | ATCC | PCS-950-040 |
| Epithelial Cell Medium | ScienCell | 4101 |
| ProstaLife basal medium | Lifeline Cell Technology | LM-0017 |
| ProstaLife LifeFactors kit | Lifeline Cell Technology | LS-1072 |
| UroLife ™ Basal Medium | Lifeline Cell Technology | LM-0054 |
| UroLife LifeFactors | Lifeline Cell Technology | LS-1115 |
| Mammary Epithelial Cell Basal Medium | ATCC | PCS-600-030 |
| Mammary Epithelial Cell Growth Kit | ATCC | PCS-600-040 |
| Mammary Life ™ Basal Medium | Lifeline Cell Technology | LM-0041 |
| MammaryLife LifeFactors Kit | Lifeline Cell Technology | LS-1088 |
| VascuLife ® Basal Medium | Lifeline Cell Technology | LM-0002 |
| VascuLife SMC LifeFactors | Lifeline Cell Technology | LS-1040 |
| iCell Neural Base Medium 1 | Fujifilm Cellular Dynamics | M1010 |
| iCell Neural Supplement A | Fujifilm Cellular Dynamics | M1032 |

Reagents

| | | Catalog |
|---|---|---|
| Reagent | Vendor | Number |
| DPBS without CaCl2, MgCl2 | Thermofisher | 14190-144 |
| | Scientific | |
| TrypLE | Thermofisher | 1260-08 |
| | Scientific | |
| ViaStain AOPI Staining Solution | Nexcelom Bioscience | CS2-0106-25mL |
| DMSO | Sigma Life Science | D2650-100mL |
| Interleukin-2, recombinant human (rhIL-2) | Sigma-Aldrich | 1147528001 |
| Interleukin-7, recombinant human (rhIL-7) | R&D Systems | 207-IL-025 |
| Simple Plex Cartridge for 72 samples, 1 analyte | Biotechne, Protein | SPCKB-PS- |
| Human-IFNg (3rd Gen) | Simple | 002574 |
| RNeasy Plus Mini Kit | Qiagen | 74124 |
| Tapestation RNA Screen Tape Ladder | Agilent | 5067-5578 |
| Tapestation RNA Screen Tape Sample Buffer | Agilent | 5067-5577 |
| Qubit RNA BR | ThermoFisher | Q10210 |
| NEBNext Ultra II Directional RNA Library Prep Kit for | NEB | E7760S |
| Illumina | | |
| SPRIselect | Beckman Coulter | B23319 |
| Qubit dsDNA HS | ThermoFisher | Q32854 |
| Tapestation High Sensitivity D5000 Reagents | Agilent | 5067-5593 |
| Tapestation High Sensitivity D5000 Ladder | Agilent | 5067-5594 |
| PhiX Control v3 | Illumina | FC-110-3001 |
| NextSeq 1000/2000 P1 Reagents (300 Cycles) | Illumina | 20050264 |
| NextSeq 1000/2000 P2 Reagents (200 Cycles) v3 | Illumina | 20046812 |
| NextSeq 2000 P3 Reagents (300 Cycles) | Illumina | 20040561 |

IFN-γ levels were determined in the culture supernatants as a measure of T cell reactivity. Bulk RNA sequencing was performed on all target cells to determine the expression of MAGE-A1, and of putative off-targets including PIEZO1, NBEAL1/NBEAL2 (which overlap in sequence) and EPN2. The TCR-T cells produced from three independent batches were systematically tested against each target cell.

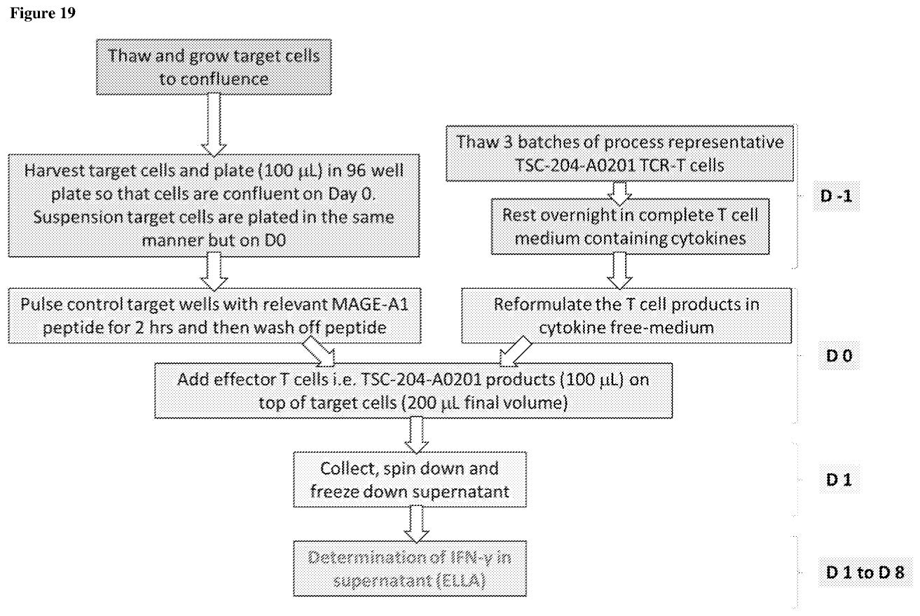

FIG. 19 outlines steps and timelines of the cytokine assay used to test off-tumor reactivity of TSC-204-A0201 TCR-T cells. Briefly, process-representative TSC-204-A0201 TCR-T cells and donor-matched UTF control T cells from three independent donors (as generated in Example 10 above) were co-cultured with HLA-A*02:01+ cancer cell lines, primary human cells, and iPSC-derived human cells from healthy donors for 20-24 hours. Culture supernatants were collected and evaluated for IFN-γ levels as a measure of T cell reactivity to the target cells. The reactivity of TSC-204-A0201 for the primary target cells was compared to multiple positive and negative controls. Multiple negative controls were used to establish the baseline IFN-γ levels in the assay. These included (1) donor-matched UTF T cells co-cultured with the same target cells, (2) TSC-204-A0201 TCR-T cells and UTF T cells co-cultured with negative control cell line Loucy, (3) TSC-204-A0201 and UTF T cells cultured alone in the absence of any targets, and (4) target cells cultured alone in the absence of any T cells. Positive controls were included to ensure that TSC-204-A0201 TCR-T cells and target cells used in the assay were functional. Target cells pulsed with the MAGE-A1-derived peptide KVLEYVIKV (SEQ ID NO: 1) were co-cultured with TSC-204-A0201 TCR-T cells to ensure that the target cells were healthy and express sufficient levels of HLA to activate TCR-T cells in response to the cognate peptide/MHC (pMHC). TSC-204-A0201 TCR-T cells were co-cultured with positive control cell line U266B1 to establish IFN-γ levels in response to endogenous MAGE-A1 and HLA expression.

Target cells and TCR-T cells were thawed. Cancer cell lines were thawed in a 37° C. water bath and washed once with their respective cell culture medium to remove cryopreservation reagents. Cells were subsequently resuspended in their respective cell culture medium and cultured following standard procedures in 75 cm² flasks (adherent target cells) or G-REX® wells (suspension cells) in a humidified incubator at 37° C. and 5% CO₂. Cells were kept at a sub-confluent state, in the exponential growth phase and passaged once or twice a week as needed. The cancer cell lines were maintained in culture for at least one passage, and no longer than 4 weeks prior to the initiation of the co-culture with TCR-T cells.

Primary human cells and iPSC-derived cells were thawed and seeded in appropriate media (Table 11) in tissue culture flasks/plates as per the manufacturer's recommendations. Media was replaced on primary cells the day after thaw with appropriate fresh media. Media was replaced thereafter as per the manufacturer's recommendations until the cells reached confluency and were ready for co-culture with TCR-T cells. Human white preadipocytes (HWP) were differentiated to adipocytes for two weeks, which were then used as target cells in the co-culture with TCR-T cells. Human cardiac myocytes (HCM) were allowed to mature in culture for 3 weeks and 6 weeks before testing them as targets in the co-culture. Peripheral blood mononuclear cells (PBMC) were used as targets immediately post-thaw.

TCR-T cells were thawed in a manner where on day-1, T cells were thawed in a 37° C. water bath and washed with cytokine-free T cell medium to remove cryopreservation reagents prior to being resuspended in T cell medium (Table 11) containing cytokines. T cells were seeded in a G-REX® 6-well plate at a density of 1E6-2E6 viable cells/mL and allowed to recover in a humidified incubator at 37° C. and 5% CO₂ for 24 hours.

Adherent target cancer cell lines were plated one day prior to setting up the co-culture (i.e., day-1). Cells were resuspended in respective media at a density of 0.5E6 cells/mL. Target cells were plated in 100 μL of their respective medium (Table 11) in 96 well flat bottom plates at 50,000 cells per well and allowed to attach overnight in the 37° C. incubator.

Primary and iPSC-derived cells were harvested as per the vendor's recommendations. Cells were resuspended in respective media at a density of 0.25E6 cells/mL. Target cells were plated in 100 μL of their respective medium (Table 11) in 96-well flat bottom plates at 25,000 cells per well and allowed to attach overnight in the 37° C. incubator. Hepatocytes were thawed and plated directly in 96 well flat bottom plates at the vendor-recommended densities on day −1. Likewise, HWP, HCM, iCell Astrocytes and iCell GABAneurons were plated directly at vendor-recommended seeding densities.

Cancer cell lines and primary cells were separately collected in tubes (2E5-3E6 cells each) and following a PBS wash, were flash frozen as pellets for RNA sequencing to determine expression of MAGE-A1 and putative off-targets of TSC-204-A0201.

Co-culture was set up on Day 0. MAGE-A1 peptide was diluted to a final concentration of 100 ng/mL in target cell-specific media. On the day of co-culture, media was gently removed from all wells receiving the peptide. Cells were peptide-pulsed with 50 uL peptide-containing media and incubated for 2-3 hours at 37° C., 5% CO2. Post incubation, cells were gently washed 3 times and 100 μL of target cell media was added. The cells were observed under the microscope to ensure that the cells had not detached.

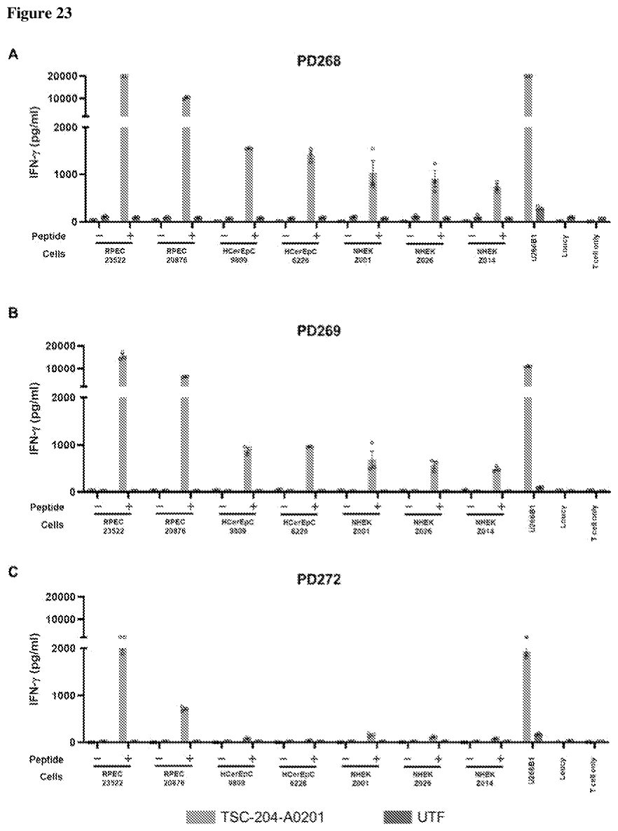

TSC-204-A0201 TCR-T cells and donor-matched UTF T cells were collected after overnight recovery, resuspended in cytokine-free T cell media (Table 11) at 0.5E6 cells/mL and 100 μL of T cell suspension was added to the appropriate wells for co-culture with target cells. For the ‘T cell only’ control, 100 μL of TCR-T cell suspension was plated along with 100 μL of cytokine-free T cell media (Table 11) except for FIG. 23 where RPEC media was added.

For the ‘target cell only’ wells, 100 μL of cytokine-free T cell media (Table 11) was added to target cell media. Co-cultures were incubated at 37° C. 5% CO₂ for 20-24 hrs.

Ella-based assessment of TCR reactivity for their cognate pMHC was performed on day 1-8. IFN-γ secretion was measured to evaluate the reactivity of TCR-T cells and donor-matched UTF T cells to cancer cell lines, primary and iPSC-derived human cells. After 20-24 hours of co-culture at 37° C. 5% CO₂, supernatants were collected and frozen at −80° C. After thawing the supernatants, IFN-γ analysis was performed on the Protein Simple ELLA (automated ELISA platform) according to the manufacturer's instructions. The raw data were exported and graphed in GraphPad Prism (v5.02).

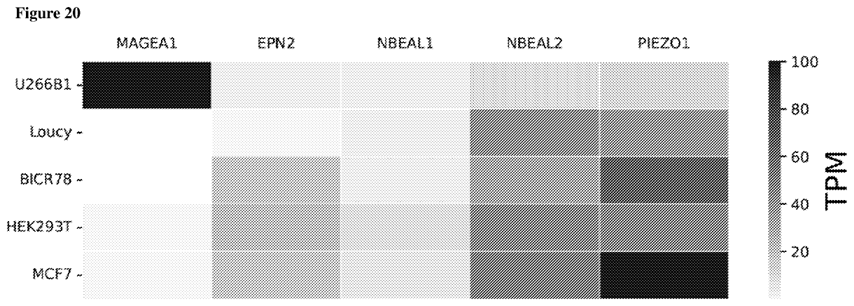

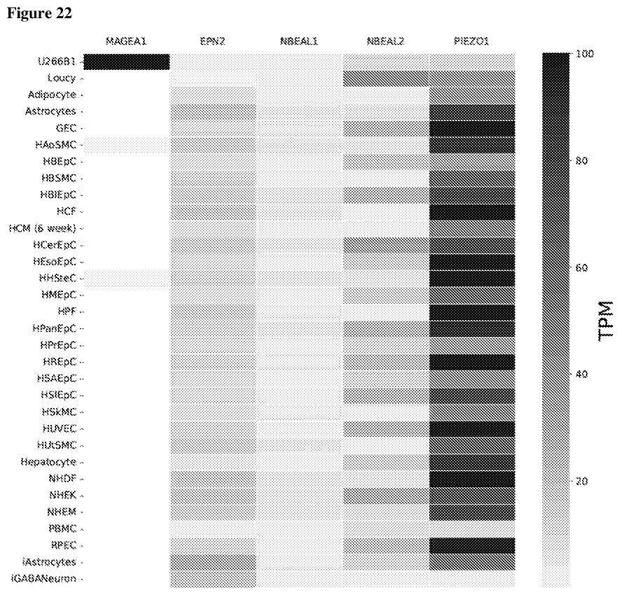

Bulk RNA sequencing was performed to determine expression of MAGE-A1 and putative off-targets of TSC-204-A0201 TCR-T cells. Total RNA was extracted from cell pellets using RNeasy® Plus Mini kits. Following manufacturer's instructions, polyadenylated RNA was isolated from up to 1 μg total RNA using NEBNext® Poly(A) mRNA Magnetic Isolation Module and directional RNAseq libraries were constructed using NEBNext® Ultra II Directional RNA Library Prep Kit for Illumina (NEB, E7760S). Libraries were then sequenced paired-end (100 bp×100 bp) on an Illumina NextSeq®2000 machine. Fastq files were preprocessed prior to alignment and gene expression calculation. The tools used for the preprocessing were Trimmomatic to cut adapter and Illumina-specific sequences from the read. Alignment was performed using hisat2 and annotated to the GRCh38 reference. The GRCh38 reference was taken from Gencode, Release 30 (GRCh38.p12). The resulting alignment files were coordinate sorted using Samtools before gene expression calculation using the subReads featureCounts function. Feature counts were converted to TPM (Transcript Per Million) by first dividing the read counts by the length of each gene in kilobases to get reads per kilobase (RPK). RPK is summed per sample then divided by 1,000,000 to give a “per million” scaling factor to generate final TPM for each sample. The color scale used in RNAseq heatmaps has TPM values of zero set to white and values above zero follow a continuous color scale up to 100 TPM as per the legend next to each heatmap in FIGS. 20 and 22.

a. Cancer Cell Line Expression of Putative Off-Targets

To demonstrate safety and lack of off-target reactivity, TSC-204-A0201 TCR-T cells and donor-matched UTF control T cells were co-cultured with a selected panel of cancer cell lines comprising BICR78, MCF7, HEK 293T and Loucy. These cell lines are HLA-A*02:01-positive, are documented to be negative for MAGE-A1 expression but express high levels of the putative off-targets of TSC-204-A0201 TCR-T cells (Scholtalbers et al. (2015) Genome Med. 7:118).

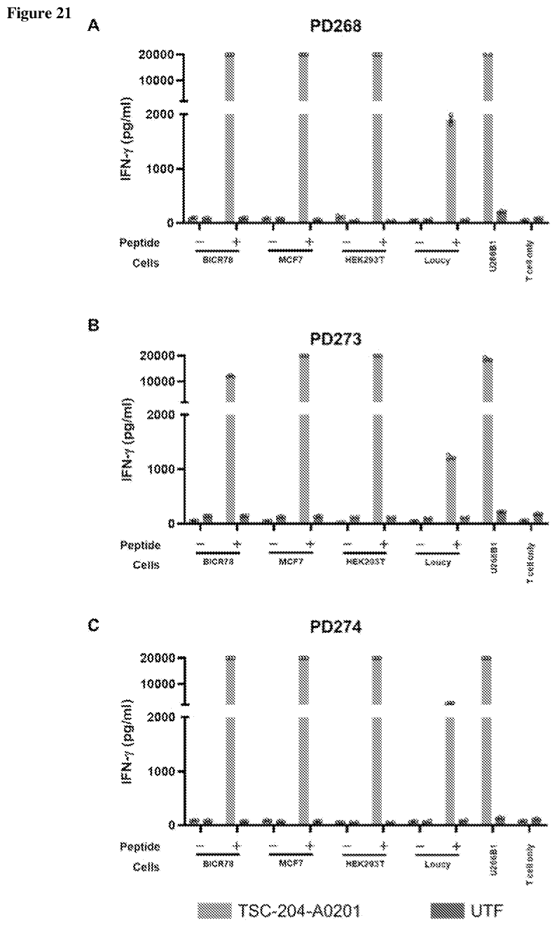

Briefly, RNAseq was performed on BICR78, MCF7, HEK 293T, Loucy and U266B1 cell lines to internally confirm the expression of putative off-targets in cancer cell lines used to confirm lack of off-target reactivity of TSC-204-A0201 TCR-T cells. As shown in FIG. 20, positive-control cell line U266B1 expressed highest levels of MAGE-A1. The expression of MAGE-A1 was undetectable (i.e. 0 Transcripts Per Million (TPM)) for Loucy and BICR78. Low but detectable expression of MAGE-A1 was observed for HEK293T (0.13 TPM) and MCF7 (0.35 TPM) cells. TSC-204-A0201 TCR-T cells, however, did not show reactivity to HEK293T and MCF7 as shown in FIG. 21. These data indicate that low levels of MAGE-A1, such as those measured for HEK293T and MCF7 controls, are not sufficient to elicit reactivity of TSC-204-A0201 TCR-T cells. Expression of putative off-targets EPN2, NBEAL1, NBEAL2 and PIEZO1 was observed in the cancer cell line panel.

b. Lack of TSC-204-A0201 TCR-T Cell Reactivity to Putative Off-Targets

To evaluate reactivity of TSC-204-A0201 TCR-T cells to putative off-targets, 3 batches of process-representative materials (PD268, PD273, and PD274) were co-cultured with BICR78, MCF7, HEK 293T and Loucy cell lines, and IFN-γ levels were determined in the culture supernatants as a measure of T cell reactivity.

FIG. 21 shows that no reactivity of TSC-204-A0201 TCR-T cells was observed for any of the cancer cell lines tested and that the positive and negative controls performed as expected. All batches of TSC-204-A0201 TCR-T cells showed robust IFN-γ secretion in response to all MAGE-A1 peptide-pulsed target cell lines. On the other hand, in the absence of peptide, baseline levels of IFN-γ expression were observed when TSC-204-A0201 TCR-T cells from the batches were co-cultured with any of the four target cell lines. The levels of IFN-γ measured were systematically comparable to the baseline levels measured with UTF control T cells, indicating that no endogenous peptide presented on the cancer cell line tested was able to stimulate IFN-γ secretion from TCR-T cells. The negative control in the assay (Loucy cells) also express high levels of putative off-targets, therebyfurther confirming that these putative off-target peptides are not naturally recognized by TSC-204-A0201 TCR-T cells.

c. Primary and iPSC-Derived Cell Expression of Putative Off-Targets
Bulk RNA sequencing was performed on target cells to determine expression of MAGE-A1 and the identified putative off-targets of TSC-204-A0201. As shown in FIG. 22, positive-control cell line U266B1 expressed the highest levels of MAGE-A1. Expression of MAGE-A1 was undetectable (i.e., 0 Transcripts Per Million (TPM)) in 28/30 primary cell types tested (FIG. 22). These data demonstrate that MAGE-A1 is predominantly not expressed in primary tissues and that MAGE-A1 expression is largely restricted to testes. Expression of putative off-targets EPN2, NBEAL1, NBEAL2 and PIEZO1 was observed in the different primary and iPSC-derived cells.
d. Lack of TSC-204-A0201 TCR-T Cell Off-Tumor Reactivity to Primary and iPSC-Derived Cells

The reactivity of TSC-204-A0201 was evaluated against a selected panel of 70 HLA*02:01-positive healthy primary human cells and iPSC-derived cells. This panel included cells from various lineages, such as epithelial, mesenchymal, endothelial, fibroblastic, muscle cells derived from multiple vital and non-vital organs, reproductive and non-reproductive organs, derived from male and female donors comprising the organs/tissues that are traditionally assessed during toxicology studies.

As shown in FIG. 22, varying levels of expression of EPN2, NBEAL1, NBEAL2 and PIEZO1 was detected in almost all cell types tested in this study. These data indicate that the primary cell panel includes an extensive collection of cell types that endogenously express putative off-targets of interest, and any reactivity (or lack thereof) is informative in assessing risk of off-tumor reactivity.

FIG. 23 provides a representative graph demonstrating that no reactivity of three batches of TSC-204-A0201 TCR-T cells and donor-matched untransfected (UTF) control T cells (PD268, PD269, and PD272) co-cultured with (1) two lots of HLA-A*02:01-positive retinal pigment epithelial cells (RPECs); (2) 2 lots of HLA-A*02:01-positive human cervical epithelial cells (HCerEpCs); or (3) 3 batches of HLA-A*02:01-positive normal human epidermal keratinocytes (NHEKs), respectively, was observed. Similar data and results were obtained for a assays in which the batches of TSC-204-A0201 TCR-T cells and donor-matched untransfected (UTF) control T cells (PD268, PD269, and PD272) co-cultured with the other primary and iPSC-derived cell types. No reactivity of TSC-204-A0201 TCR-T cells was observed for any of the primary and iPSC-derived cell types tested in this study. In addition, the positive and negative controls performed as expected, demonstrating the validity of these assays (see, for example, FIG. 23).

Taken together, these data further confirm that TSC-204-A0201 TCR-T cells fail to react with primary samples lacking any significant MAGE-A1 expression, even when the cells endogenously express a putative off-target of the therapeutic TCR and indicate that reactivity of the TCR-T cells are restricted to cancer cells expressing MAGE-A1 and HLA-A*02:01.

### Example 13: Lack of TSC-204-A0201TCR-T Cell Oncogenicity

An in vitro oncogenicity assay was performed to further confirm that manufacture of

TSC-204-A0201 (e.g., during translation of transposase mRNA into enzymatic protein within the cell and facilitates the transposition of TSC-204-A0201 npDNA transposon at 5′-TTAA-3′ sites within the host genome) does not result in vector-induced insertional mutagenesis and oncogenic transformation of the TCR-T cells (e.g., that translation of transposase mRNA into enzymatic protein within the cell and facilitates transposition of TSC-204-A0201 npDNA transposon at 5′-TTAA-3′ sites within the host genome) (Wu et al. (2011) Front Med. 5:356-371; Manfredi (2020) Front. Immunol. 11:1689; Nobles et al. (2020) J. Clin. Invest. 130:673-685; Micklethwaite et al. (2021) 138:1391-1405).

To evaluate cytokine-dependency, process-representative TSC-204-A0201 TCR-T cells and donor-matched untransfected (UTF) control T cells were cultured in the presence or absence of cytokines (IL-2 and IL-7) for 5 days and analyzed for cell survival and proliferation. UTF control T cells that have not undergone any electroporation and transposition, and hence, are devoid of any insertional mutagenesis served as controls. A positive control of T cell proliferation was included by stimulating the TSC-204-A0201 TCR-T cells and donor-matched UTF control T cells with ImmunoCult (IC) Human CD3/CD28/CD2 T cell Activator.

The data demonstrate that, similar to donor-matched UTF control cells, TSC-204-A0201 TCR-T cells exhibited lower survival and proliferation when cultured in the absence of cytokines compared to T cell survival and proliferation when cultured in the presence of cytokines. Further, in the absence of cytokines, both proliferation and survival of TSC-204-A0201 TCR-T cells was similar or lower to that of the donor-matched UTF cells. Together these data indicate lack of cytokine-independent survival or (hyper) proliferation of TCR-T cells.

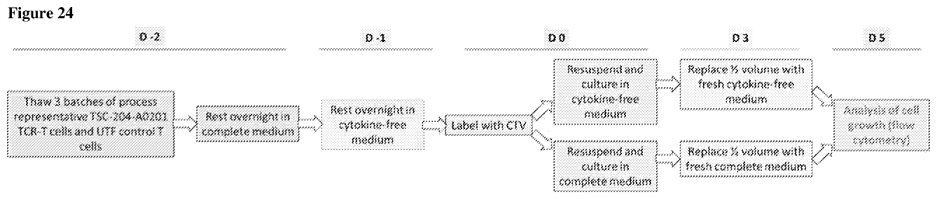

In particular, the oncogenicity assay was performed according to the steps and timeline depicted in FIG. 24. TSC-204-A0201 TCR-T cells and donor-matched UTF T cells were thawed and maintained in cytokine-containing culture medium (Table 12) on day-2.

TABLE 12
Reagents
Prepared T cell media

| Media | Components | Stock (mL) | Catalog Number |
|---|---|---|---|
| “T cell medium” XVIVO | X-VIVO 15 | 1000 | Lonza#04-418Q |
| 15 + 5% HI-HS + 1% | Human Serum (Heat- | 50 | Sigma-Aldrich#H3667-100 |
| P/S +1XGLUT) | Inactivated) | | |
| | Pen Strep | 10 | Gibco#15149-122 |
| | GlutMAX (100x) | 10 | FisherScientific#35050061 |
| “T cell medium with Cytokines” | T cell medium | 300 | NA |
| (IL-2 and IL-7) | Human IL-2 (5 ug/mL) | 1.5 | Sigma-Aldrich#11147528001 |
| | Human IL-7 (10 ug/mL) | 0.150 | R&D Systems#207-IL-025 |

Media composition for oncogenicity assay

| | Cytokines (IL-7 & IL-2) | | | ImmunoCult | | |
|---|---|---|---|---|---|---|
| | | 2x | 1x | | | 1x |
| | | concentration | concentration | | 2x concentration | concentration |
| Media to Prepare | | preparation | preparation | | preparation | preparation |
| T Cell Media #1 (10 mL) | − | NA | NA | − | NA | NA |
| T Cell Media #2 (10 mL) | + | 100 μL IL-2 | 50 μL IL-2 | − | NA | NA |
| | | 10 μL IL-7 | 5 μL IL-7 | | | |
| T Cell Media #3 (10 mL) | + | 100 μL IL-2 | 50 μL IL-2 | + | 40 μL | 20 μL |
| | | 10 μL IL-7 | 5 μL IL-7 | | | |

Media and supplements

| Media | Vendor | Catalog Number |
|---|---|---|
| X-VIVO 15 | Lonza | 04-418Q |
| Human Serum (Heat-Inactivated) | Sigma-Aldrich | H3667-100 |
| GlutaMAX (100x) | Fisher Scientific | 35050061 |
| Penicillin-Streptomycin | Gibco | 15140-122 |
| Human IL-2 (5 ug/mL) | Sigma-Aldrich | 11147528001 |
| Human IL-7 (10 ug/mL) | Sigma-Aldrich | 207-IL-025 |

Reagents

| Reagent | Vendor | Catalog Number |
|---|---|---|
| Easysep ™ | Stem Cell Technologies | 20144 |
| ImmunoCult CD3/CD28/CD2 | Stem Cell Technologies | 10970 |
| Cell Trace Violet | Invitrogen | C34557 |
| eFluor 660 Viability Dye | eBioscience | 65-0864-14 |
| DMSO | Sigma Life Science | D2650-100 mL |
| Count Bright Beads | Invitrogen | C36950 |

Specifically, T cells were thawed in a 37° C. water bath and washed twice with cytokine-free T cell medium (Table 12) to remove cryopreservation reagents prior to being resuspended in T cell medium containing cytokines. T cells were seeded in a G-REX® 6-well plate at a density of 1E6 viable cells/ml and allowed to recover in a humidified incubator at 37° C. and 5% CO₂ for 24 hours.

After allowing the T cells to recover for 24 hours, the T cells were washed in cytokine-free culture medium and then maintained in cytokine-free culture medium on day −1. T cells were seeded in a G-REX® 6-well plate at a density of 1E6 viable cells/ml and allowed to rest in a humidified incubator at 37° C. and 5% CO₂ for 24 hours prior to oncogenicity evaluation.

After allowing the T cells to rest for an additional 24 hours, the T cells were stained with CTV (cell trace violet) proliferation dye on Day 0 and were cultured in the following media: 1) cytokine-free medium (absence of IL-2 and IL-7); 2) cytokine-containing medium (presence of IL-2 and IL-7); and 3) cytokine-containing medium with IC added as a positive control to stimulate proliferation.

Specifically, on day 0 (48 hours after thaw and recovery), TSC-204-A0201 TCR-T cells and donor-matched UTF control T cells were washed twice in cytokine-free T cell medium. 4E6 viable cells were stained with CTV proliferation dye at a concentration of 2.5 μM/ml in 1 ml of PBS (except for TSC-204-A0201 TCR-T test articles derived from donor PD272, where 1.6E6 viable cells were stained with CTV proliferation dye) for 10 minutes at room temperature protected from light. After washing twice with T cell medium, 8E4 viable T cells were then seeded into 96-well U-bottom in 200 μl of media. To help reduce evaporation, 200 μl of PBS was added to each well along the border of experimental wells. Cells were confirmed to express similar levels of CTV.

On day 3, half of the T cells were passaged in the appropriate media. Specifically, T cells in the 96-well U-bottom plate were resuspended and half of the cells were transferred to a new 96-well U-bottom plate and passaged in the appropriate media. Cells were cultured for an additional 2 days and were analyzed by flow cytometry.

On day 5, the numbers of viable cells and proportion and numbers of proliferated T cells were determined by flow cytometry. Specifically, T cells were harvested, washed with PBS, and stained with fixable viability dye eFlour 660 according to the manufacturer's instructions. T cells were then resuspended in 100 μl/well of EasySep® buffer with 1.02E3 Count Bright beads/well. T cells were analyzed by flow cytometry using the CytoFLEX flow cytometer (Beckman Coulter) (acquisition volume: 90 μl, sample flow rate 60 μl/min) and data were analyzed by FlowJo™ (Treestar) (version 10.6.2).

Numbers of viable and proliferating cells were quantified using a gating strategy. Briefly, precision counting beads were first separately gated out using irrelevant fluorescence channels for cell count normalization purposes. Forward scatter area (FSC-A) vs. side scatter area (SSC-A) density plots were then used to identify cells and to exclude debris. Within this ‘Lymphocyte’ population, single T cells were separated from doublets using the FSC-height vs. FSC-Area density plots. Next, eFlour 660 viability dye staining within the ‘single cell’ population distinguished viable eFlour 660-negative T cells from non-viable eFlour 660-positive T cells. Dividing T cell generations were then separated from non-dividing T cells by a reduced fluorescence intensity of CTV proliferation dye within the ‘Live Cells’ population. Absolute count of viable cells (viability dye eFlour 660-negative) and proliferating cells were normalized to absolute bead counts. Normalized counts of viable and dividing cells obtained on day 5 were multiplied by two to compensate for passaging the cells on day 3.

Raw data were exported, graphed, and analyzed in GraphPad Prism (v5.02). Statistical differences between values of TSC-204-A0201 TCR-T cells and their donor-matched non-edited UTF control T cells, and between test conditions of culturing cells in the absence of cytokines or in the presence of cytokines were determined by 2-way ANOVA (Sidak correction for multiple comparisons). *** p≤0.0001, *** p≤0.001, ** p≤0.01, * p≤0.05, ‘ns’ means not significant p>0.05.

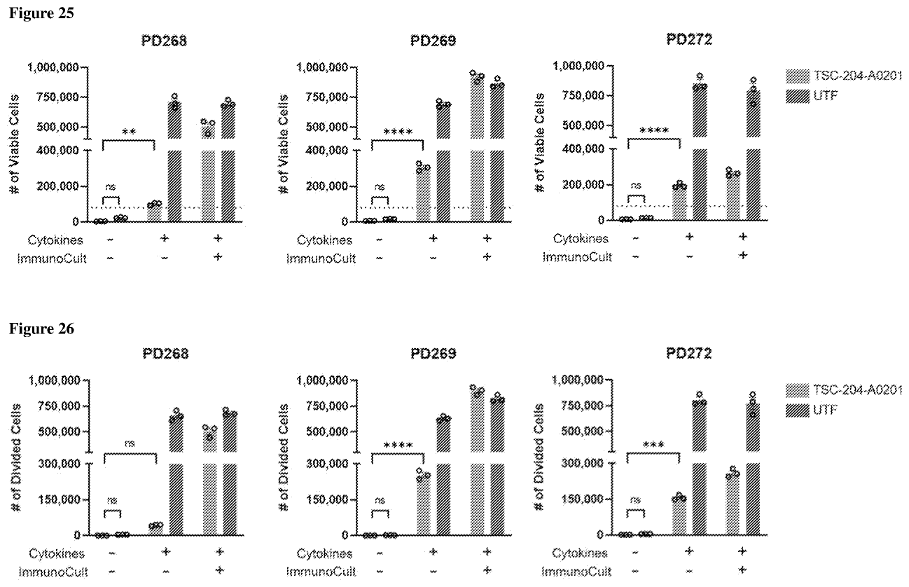

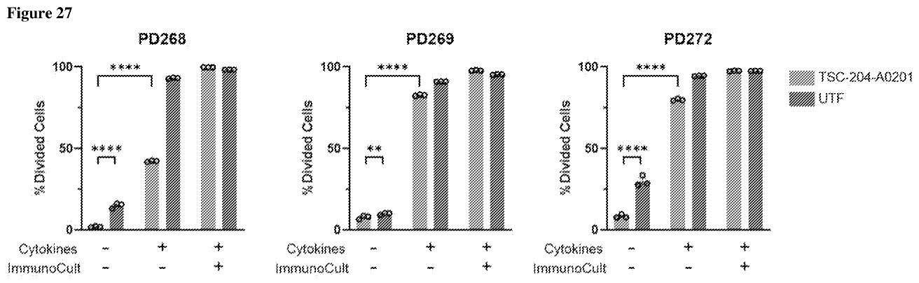

FIGS. 25-27 show that positive controls exhibit highest levels of proliferation. T cell survival and proliferation in vitro was dependent on stimulation through the TCR and/or cytokines. TSC-204-A0201 TCR-T cells and donor-matched UTF control T cells stimulated with ImmunoCult (IC) Human CD3/CD28/CD2 T cell Activator in the presence of cytokines served as positive controls for the oncogenicity assay (the “Cytokines+, ImmunoCult+” condition in FIGS. 25-27). Under these culture conditions, engineered TCR-T cells and their UTF control counterparts from all batches tested expanded significantly, producing a high number of live cells (FIG. 25). The proportion of proliferating cells was also high with >95% of the live cells undergoing proliferation during the 5 days of the experiment (FIGS. 26 and 27). These data confirmed that both TSC-204-A0201 TCR-T cells and UTF control T cells used in the assay were viable and functional.

By contrast, negative controls exhibited no hyperproliferation since donor-matched UTF control T cells did not undergo electroporation or transposition (thus devoid of any insertional mutagenesis) and these cells represent an ideal comparator to test the cytokine-dependency for cell survival and proliferation of TSC-204-A0201 TCR-T cells. As expected, UTF control T cells cultured in the presence of cytokines were able to survive and proliferate as demonstrated by high numbers of total viable T cells similar to the trends observed for the total numbers of T cells obtained in the positive control condition (FIG. 25). Of those cells, the trends for proportion and total numbers of cells engaging in cell cycle was also comparable to the trends observed in the positive control culture condition (FIGS. 26 and 27).

In the absence of cytokines, however, the UTF control T cells failed to survive and a significant reduction in numbers of total viable cells was observed when compared to UTF control T cells cultured in the presence of the cytokines on day 5 (FIG. 25). The numbers of viable cells in the absence of cytokines were lower than the day 0 seeding cell number of 80,000 cells (dotted line in FIG. 25) confirming that the cells died. In addition, a reduction in the proportions and numbers of proliferating cells was observed for all donors tested in the absence of cytokines compared to UTF control T cells cultured in the presence of cytokines (FIGS. 26 and 27). Taken together, these data indicate that, under the conditions of the experiments, unedited UTF control T cells of the batches tested do not survive and only engage in limited proliferation in the absence of cytokines.

TSC-204-A0201 showed cytokine-dependent survival and proliferation. Similar to UTF control T cells, TSC-204-A0201 TCR-T cells were able to survive and proliferate in the presence of cytokines (cytokine-dependent) (FIGS. 25-27).

In the absence of cytokines, none of the TSC-204-A0201 batches were able to survive or expand. TSC-204-A0201 TCR-T cells showed a clear reduction in viability and proliferation when compared to TSC-204-A0201 TCR-T cells cultured in the presence of the cytokines (FIGS. 25-27).

Taken together, these data confirm that TSC-204-A0201 TCR-T cells remained dependent on cytokines and TCR-mediated signals to survive and expand similarly to non-edited cells such that the engineering process did not produce insertional mutagenesis resulting in oncogenic transformation of the TCR-T cells.

### Example 14: MAGE-A1 Target Expression in Normal Human Tissues

As described above, MAGE-A1 is a cancer/testis protein that is produced during embryonic development but is virtually absent in all normal adult tissues except testis (Gjerstorff et al. (2007) Hum. Reprod. 22:953-960), which is an immune privileged tissue (Li et al. (2012) Front Immunol. 3:152; Hedger (2014) Knobil Neill Physiol. Reprod. 2015:805-892). To confirm that MAGE-A1 expression is enriched in testes, but not other normal tissues, cDNA arrays were purchased from a commercial vendor containing cDNA from 48 different normal (non-cancer) tissue sections and an additional 24 different subsection of normal (non-cancer) brain tissue and assayed for MAGE-A1 and GAPDH gene expression using a multiplexed MAGE-A1 and GAPDH qPCR assay described below, in technical triplicates.

Specifically, cDNA arrays in 96-well plate format were purchased from Origene and handled according to manufacturer instructions. A master mix consisting of TaqMan® Fast Advanced Master mix (ThermoFisher, cat #4444557), Nuclease-free water (Invitrogen, cat #AM9937), MAGE-A1 and GAPDH TaqMan probes, were aliquoted into the 96-well cDNA array plate, 20 μL per well. Gene expression was measured on a QuantStudio 7 and Cq values were quantified, analyzed, and graphed for both Normal tissue (OriGene, cat #HMRT304) and Normal brain (OriGene, cat #HBRT301) cDNA arrays. Three individual plates were assayed for each array (n=3 technical replicates).

Cq values were obtained from the QuantStudio 7 for each cDNA array. Cq values for MAGE-A1 was normalized to GAPDH to obtain the delta CT (ΔCt). The ΔCt values were then quantified using the 2\hat{ }-ΔCt method to indicate levels of MAGE-A1 expression relative to levels of GAPDH. Quantification of individual replicates are graphed with the mean±standard error indicated.

In some embodiments, a cDNA from testes was prepared and added as a positive control for the assay. Testes RNA (OriGene Technologies, cat #CR560016) was synthesized to cDNA using the First Strand cDNA Synthesis kit (OriGene Technologies, cat #NP100042): 4 μL of 5× cDNA Synthesis Mix (60073), 1 μL of Reverse Transcriptase (60074) and 9 μL Nuclease-Free Water (60064) master mix was made and combined with 6 μL of testes RNA (5 micrograms) in a 0.2-mL thin-walled PCR tube (total volume of 20 μL). The tube was vortexed, gently centrifuged for 10 seconds and placed in a thermocycler (BIO-RAD T100 Thermal Cycler) for synthesis under the following program: 1 cycle: 22 degrees Celsius for 5 minutes; 1 cycle: 42 degrees Celsius for 30 minutes; 1 cycle: 85 degrees Celsius for 5 minutes; 4 degrees Celsius hold.

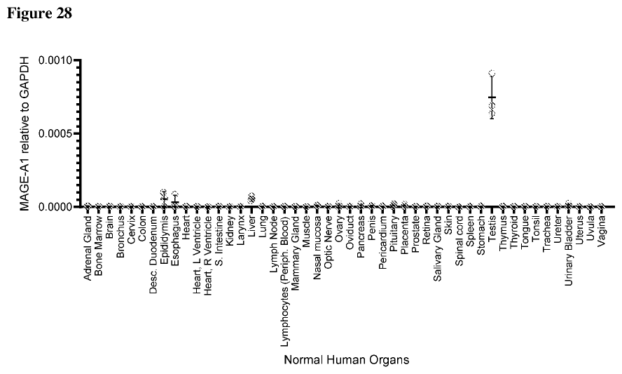

FIG. 28 shows MAGE-A1 expression across 48 tissue types measured from the TissueScan-Normal human tissue array. Individual replicates (n=3) of MAGE-A1 expression normalized to GAPDH expression are plotted (circles) and the mean and standard error are indicated with black bars. Of the 48 normal tissues examined, only testes exhibited high MAGE-A1 expression across 3 technical replicates. MAGE-A1 expression was undetectable across 3 technical replicates for 40 tissues. These data concur with the abundant literature supporting that MAGE-A1 is an ideal tumor-associated protein being absent from non-testes normal tissues.

Liver ostensibly showed low levels above background in FIG. 28, but result was due to significantly lower GAPDH levels for this sample used for normalizing purposes as compared to other samples. For four tissues (bladder, pancreas, ovary, and esophagus), low but detectable signal was observed in only 1 out of 3 technical replicates. Due to the lack of reproducibility across replicates, these are believed to represent background/false positive signal. Three remaining tissues (placenta, pituitary, and epididymis) showed detectable, albeit low, signal across 2 out of 3 technical replicates. The signal from placenta is not an issue as pregnant women are not eligible for TSC-204-A0201 therapy. The signal from epididymis is believed to reflect cross-contamination from testes during gross tissue preparation since similar observations were made while testing other testes-restricted genes that were detected in the epididymis. The signal from pituitary, while very low (Cq=37-38), was observed across two replicates.

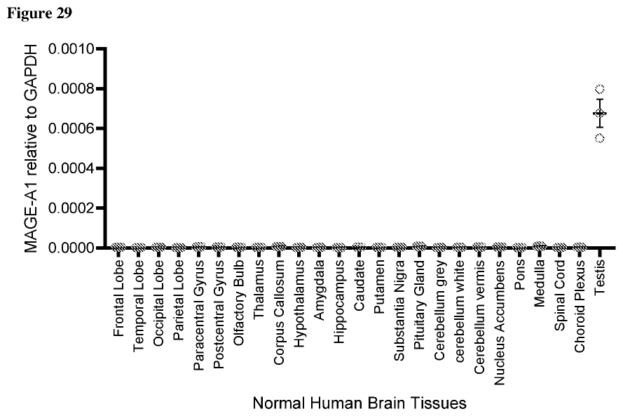

To further evaluate this result, additional expression analysis on a second cDNA array design specific to brain was performed. FIG. 29 shows MAGE-A1 expression across 24 tissue sub-types measured from the TissueScan-human brain tissue array. Individual replicates (n=3) of MAGE-A1 expression normalized to GAPDH expression are plotted (circles) and the mean and standard error are indicated with black bars. cDNA from testes (prepared separately as described above) was added to each plate as a positive control for the assay. MAGE-A1 expression was undetectable across 3 technical replicates for all 24 tissues examined, including pituitary gland. Consistent with data from FIG. 28, testes exhibited high MAGE-A1 expression across 3 technical replicates, indicating that the MAGE-A1 assay was functional across all plates. GAPDH Cq values from the samples were all within the high-confidence range of the assay and consistent with levels observed for the normal tissue array. This indicates that gene expression is reliably quantifiable from this brain-derived cDNA array. Brain sections in this array consistently appeared negative for MAGE-A1 expression.

In summary, consistent with prior literature (van der Bruggen et al. (1991) Science 254:1643-1647; Obenhaus et al. (2015) Nat. Biotechnol. 33:402-407) and publicly available RNA-sequencing data, MAGE-A1 expression in normal tissue is largely restricted to testes. These gene expression analyses indicate that MAGE-A1 represent a safe target for T cell-based therapies with low risk for on-target off-tumor reactivity.

### Example 15: DN-TGFβRII Renders Engineered T Cells Resistant to TGFβ-Mediated Suppression

As described herein, engineered T cells may comprise additional elements in addition to TCRs of interest. For example, engineered TCR-T cells may include pan T cells engineered with a transposon containing a murine stem cell virus (MSCV) promoter driving the expression of the α and β chains of the recombinant TCR and of the α and β chains of CD8. The α and β chains of the TCR, along with the α and β chains of CD8 may be encoded by a single mRNA molecule. Post-translational processing by the self-cleaving peptide P2A results in four separate polypeptides.

The amino acid sequence of the constant regions of the α and β chains of the therapeutic TCR components may be optimized to promote the expression and correct a/β pairing of the therapeutic TCR used in TSC-204-A0201. The goal of these modifications was to promote the expression and correct a/β pairing of the therapeutic TCR in the engineered T cells.

To ensure that helper T cells contained in TSC-204-A0201 TCR-T cells recognize the target cells and get functionally engaged, ORFs encoding the CD8α and CD8β co-receptors may be delivered to the engineered T cells along with the therapeutic TCR. The CD8a co-receptor may be modified to include an epitope, such as a 16-amino acid epitope at its N-terminus corresponding to the portion of CD34 that is recognized by a monoclonal antibody-clone QBEND10. Including an epitope such as this epitope enables tracking engineered T cells in vitro and in vivo.

In some embodiments, engineered TCR-T cells may comprise additional elements, optionally regulated through a second expression cassette. Such a second expression cassette may be under the control of different promoter, such as the human elongation factor 1a (EF1α) promoter. In this manner, two genes may be encoded by a single mRNA molecule and post-translationally processed into two polypeptides.

In some embodiments, engineered TCR-T cells may comprise a selection marker, optionally wherein the element is expressed in the engineered T cells from a second expression cassette in the vector. In some embodiments, the selection marker may be a mutated form of dihydrofolate reductase (DHFRdm) protein. This protein provides the engineered cells with a selective advantage upon exposure to methotrexate (MTX), which enables enrichment of engineered cells during the TCR-T cell manufacturing process.

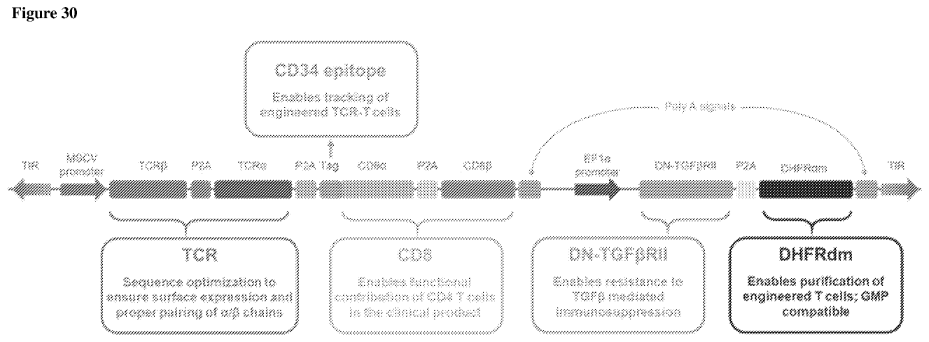

In some embodiments, engineered TCR-T cells may comprise a dominant negative form of type II TGFβ receptor (DN-TGFβRII), optionally wherein the element is expressed in the engineered T cells from a second expression cassette in the vector. DN-TGFβRII is a decoy receptor that retains the extracellular and transmembrane domains of wild-type TGFβRII but lacks the intracellular kinase domain (Wieser et al. (1993) Mol. Cell Biol. 13:7239-7247; Bollard et al. (2002) Blood 99:3179-3187). As a result, expression of DN-TGFβRII renders engineered T cells resistant to the immunosuppressive effects of TGFβ (Dahmani and Delisle (2018) Cancers (Basel) 10:194. FIG. 30 provides a representative schematic of a construct encompassed by the present discosure.

To characterize the contribution of a DN-TGFβRII element, TSC-204-A0201 TCR-Tcells were engineered with a bench-scale adaptation of the clinical process (referred to as “process-similar”) using transposon vectors identical to the final transposon vector, as described above in Example 10, or with a version lacking the DN-TGFβRII element. The TCR-T cells were then tested functionally in the presence or absence of 5 ng/ml TGFβ (corresponding to a physiological level; Khan et al. (2012) BMC Res. Notes 5:636), to evaluate the ability of the engineered T cells to maintain their target-dependent functions in the presence of TGFβ. TGFβ-treated TCR-T cells were cultured with various target cells (either expressing the cognate peptide/MHC or not) and multiple endpoints were assessed, including target-dependent secretion of cytokines, target-dependent T cell proliferation, and cytotoxic activity.

Process-similar TCR-T cells were engineered. Pan T cell was first isolated. PBMCs were isolated from Leukopacs of healthy donors using Ficoll-based density centrifugation following standard procedures. Subsequently, pan-T cells were isolated from PBMCs using a magnetic bead-based system (Easysep™ Human T cell isolation kit, StemCell Technologies) according to instructions of the manufacturer. Pan-T cells were then frozen down and stored as described below.

T cell was then nucleofected and activated. After thawing, pan-T cells were washed once with cytokine free pan-T cell thawing medium and were then resuspended at 1-2E6 cells/mL in complete Pan-T cell thawing medium (Table 13).

TABLE 13
Cell lines and reagents
Target cells

| Target cell | | | MAGE-A1 | HLA-A*02:01 | HLA-C*07:02 |
|---|---|---|---|---|---|
| line | Indication | Source | (TPM) | (HLA-A TPM) | (HLA-C TPM) |
| SW1271 | Lung | ATCC | + (84.02) | + (385.75) | + (706.97) |
| HS936T | Melanoma | ATCC | + (69.86) | + (901.57) | + (344.14) |
| A101D | Melanoma | ATCC | + (136.79) | − (342.72) | + (728.71) |
| AU565 | Breast | ATCC | + (72.46) | + (44.95) | − (54.85) |
| U266B1 | Myeloma | ATCC | + (241.7) | + (321.72) | + (509.05) |
| LOUCY | T lymphoblast | ATCC | − (0.02) | + (1578.5) | + (671.48) |

Expression as transcripts per million (TPM) of MAGE-A1, of HLA-A*02:01 and of HLA-C*07:02 in the
different cell lines are indicated (source: cancer cell line encyclopedia, Broad Institute).
Culture media compositions

| Media | Cells | Components | V stock (mL) | Vendor (Catalog Number) |
|---|---|---|---|---|
| Cytokine-free | Pan-T cells | RPMI1640 | 500 | ATCC 30-2001 |
| Pan-T cell | (thaw& | | | |
| thawing | wash) | Fetal Bovine Serum | 50 | Thermofisher Scientific |
| medium | | | | #A3840001 |
| | | Penicilin/Streptomycin | 5 | Thermofisher Scientific |
| | | | | #15149-122 |
| Complete | Pan-T cells | Cytokine-free Pan-T cell | 200 | n/a |
| Pan-T cell | (recovery | thawing medium | | |
| thawing | from thaw) | Human IL-2 (5 μg/mL) | 1 | Sigma-Aldrich #11147528001 |
| medium | | Human IL-7 (10 μg/mL) | 0.1 | R&D Systems #207-IL-025 |
| | | Human IL-15 (10 | 0.1 | R&D Systems #247-IL-025 |
| | | μg/mL) | | |
| Cytokine-free T | TCR-T cells | X-VIVO 15 | 1000 | Lonza #04-418Q |
| cell medium | (co-culture) | Human Serum (Heat- | 50 | Sigma-Aldrich #H3667-100 |
| | | Inactivated) | | |
| | | Pen Strep | 10 | Gibco #15149-122 |
| | | GlutaMAX (100x) | 10 | FisherScientific #35050061 |
| Complete T cell | TCR-T cells | Cytokine-free T cell | 200 | n/a |
| medium | (thawing, | medium | | |
| | resting) | Human IL-2 (5 μg/mL) | 1 | Sigma-Aldrich #11147528001 |
| | | Human IL-7 (10 μg/mL) | 0.1 | R&D Systems #207-IL-025 |
| RPMI-based | AU565 | RPMI1640 | 500 | ATCC30-2001 |
| medium 10% | LOUCY | Fetal Bovine Serum | 50 | Thermofisher Scientific |
| FBS | | | | #A3840001 |
| | | Penicilin/Streptomycin | 5 | Thermofisher Scientific |
| | | | | #15149-122 |
| RPMI-based | U266B1* | RPMI1640 | 500 | ATCC30-2001 |
| medium 15% | | Fetal Bovine Serum | 75 | Thermofisher Scientific |
| FBS | | | | #A3840001 |
| | | Penicilin/Streptomycin | 5 | Thermofisher Scientific |
| | | | | #15149-122 |
| RPMI-based | SW1271 | RPMI1640 | 500 | ATCC30-2001 |
| medium 20% | | Fetal Bovine Serum | 100 | Thermofisher Scientific |
| FBS | | | | #A3840001 |
| | | Penicilin/Streptomycin | 5 | Thermofisher Scientific |
| | | | | #15149-122 |
| | HS936T, | DMEM | 500 | Thermofisher Scientific |
| | A101D | | | #11965092 |
| DMEM-based | | Fetal Bovine Serum | 50 | Thermofisher Scientific |
| medium 10% | | | | #A3840001 |
| FBS | | Penicillin/Streptomycin | 5 | Thermofisher Scientific |
| | | | | #15149-122 |

* While U266B1 were maintained in RPMI1640 15% FBS 1% PS, RPMI1640 10% FBS 1% PS was used for
coculturing with T cells
Flow cytimetry staining reagents

| Antibody | Clone | Vendor | Fluorochrome | Catalog Number |
|---|---|---|---|---|
| near-IR fluorescent | N/A | Thermofisher | Near-IR | L10119 |
| reactive dye | | Scientific | (APC-Cy7) | |
| CD34 | QBEND/10 | Thermofisher | Biotinylated | MA5-16924 |
| | | Scientific | | |
| Streptavidin | N/A | BD Bioscience | BV421 | 563259 |
| CD4 | RPA-T4 | BioLegend | FITC | 300538 |
| CD8β (panel 1) | SIDI8BEE | Thermofisher | PE | 12-5273-42 |
| Or | | Scientific | | |
| TGFβRII (panel 2) | W17055 | BioLegend | PE | 399704 |
| MAGE-A1/HLA- | NA | IMMUDEX | APC | Custom (HLA- |
| A*02:01 Or | | | | A*02:01/KVLEYVIKV |
| MAGE-A1/ | | | | (SEQ ID NO: 1)) |
| HLA-C*07:02 | | | | Or |
| Dextramer | | | | Custom (HLA- |
| | | | | C*07:02/VRFFFPSL |
| | | | | (SEQ ID NO: 43)) |

Antibody/staining reagents

| Reagent | Clone | Vendor | Fluorochrome | Catalog Number |
|---|---|---|---|---|
| Human | N/A | Biolegend | N/A | 422302 |
| Trustain Fc | | | | |
| Block | | | | |
| Far Red | N/A | Thermofisher | Far Red (APC) | L10120 |
| Fixable live | | Scientific | | |
| dead dye | | | | |
| CD34 | QBEND/10 | Thermofisher | Biotinylated | MA5-16924 |
| | | Scientific | | |
| Streptavidin | N/A | BioLegend | Alexa488 | 405235 |
| CD4 | RPA-T4 | BioLegend | PE-Cy7 | 300538 |
| CD3 | UCHT1 | BD Bioscience | Brilliant Violet 650 | 563852 |

Pan-Ts were seeded into 6-well G-REX® plates and were allowed to recover from thaw for 2 hours at 37° C. 5% CO₂. Subsequently, Pan-T cells were washed once with DPBS and were resuspended in P3 primary buffer (Lonza) at 1E8 cells/mL. Transposase mRNA (Aldevron) and nanoplasmid (produced in house, plasmid ID PNNVD136; PNNVD166; PNNVD142 and PNNVD162) were added at a final concentration of 100 μg/mL and 20 μg/mL, respectively. The cell suspension was then distributed into electroporation cartridges (cartridge size L, Lonza; 100 μL per cartridge) and was electroporated using the Amaxa 4D nucleofector and electroporation program FI-115 according to instructions of the manufacturer. After electroporation, cells were transferred to wells of a 6-well G-REX® plate containing prewarmed Immunocult-XF T cell expansion medium (StemCell Technologies) and were rested at 37° C. 5% CO₂. After ~20 hours resting, one volume of Immunocult-XF T cell expansion medium containing 2× cytokines (IL-2, IL-7 and IL-15) and 2× T cell activator (Immunocult™ Human CD3/CD28/CD2 T cell activator, StemCell Technologies) was added, resulting in a 1× concentration of cytokines (IL-2:25 ng/ml; IL-7:5 ng/ml; IL-15:5 ng/ml), and a final concentration of T cell activator of 10 μL/mL.

T cell expansion and methotrexate selection was performed. Briefly, T cells were split 1:1 into fresh process-representative expansion medium (Table 13) every 2-3 days using regular or tall 6-well G-REX® plates, and cytokines (IL-2, IL-7 and IL-15) were spiked in assuming that all cytokines had been depleted. To initiate methotrexate selection, cells were spun down at 300 g for 5-10 minutes and were resuspended at 1E6 live cells/mL in process-representative expansion medium. Methotrexate was added at a final concentration of 0.1 μg/mL. After 2-3 days, cells were split 1:1 in methotrexate containing process-representative expansion medium. After 5 days of selection, cells were spun down, were resuspended in complete T cell medium (Table 13) and were expanded for an additional 3-4 days. Finally, cells were frozen down and stored. Cells were washed once with cold EasySep®, resuspended in CryoStor® CS10 (StemCell Technologies) at 10-30E6 cells/mL and distributed into cryovials (1 mL or 5 mL). Vials were placed into Cool Cells and were incubated at −80° C. for 1-3 days. Subsequently, cells were transferred to liquid nitrogen for long term storage.

Effector cells were prepared. T cells were thawed in a 37° C. water bath and washed once with cytokine-free T cell medium to remove cryopreservation reagents prior to being resuspended in complete T cell medium. T cells were then seeded in a G-REX® 6-well plate at a density of 1E6-2E6 live cells/mL and allowed to recover in a humidified incubator at 37° C. and 5% CO₂ for 16-24 hour prior to co-culturing. On the day of co-culture, T cells were harvested, washed, and resuspended in cytokine-free T cell medium (Table 13) at the desired cell density.

Similarly, target cells were prepared. Cancer cell lines were thawed in a 37° C. water bath and washed once with their respective cell culture medium to remove cryopreservation reagents. Cells were subsequently resuspended in their respective cell culture medium and cultured following standard procedures in 75 cm² or 150 cm² flasks in a humidified incubator at 37° C. and 5% CO₂. Cells were passaged once or twice a week as needed. The cancer cell lines were maintained in culture no longer than 4 weeks prior to the initiation of the co-culture with T cells.

Co-culture was prepared. Adherent target cells were plated one day before setting up the co-culture. For the Incucyte®-based cytotoxicity assay, target cells were plated in 100 μL of their respective medium (Table 13) in 96-well flat bottom plates at 5E3 cells per well (AU565) or 7E3 cells per well (HS936T, SW1271) to achieve a target cell density of ~1E4 cells per well after 20-24 hour incubation at 37° C. 5% CO₂. Note that the seeding densities were adapted according to the variable growth rates of these cell lines. For cytokine and proliferation assays, adherent target cells were plated in 96 well flat bottom plates at 2.5E4 cells per well (A101D) or 3.5E4 cells per well (HS936T, SW1271) to achieve a target cell density of 5E4 cells per well after 20-24 hour incubation at 37° C. 5% CO₂. Non-adherent target cells (U266B1, Loucy) were plated in U-bottom plates the day of initiation of the co-culture, following assay specific instructions.

Two batches of TSC-204-A0201 TCR-T cells were analyzed. Donor-matched DN-TGFβRII-negative TCR-T cells were used as negative controls. A panel of target cancer cell lines naturally expressing MAGE-A1, HLA-A*02:01 were identified and used in co-culture assays with the batches of TCR-T cells in the presence or absence of exogenous TGFβ cytokine (5 ng/mL). The target-dependent function of the TCR-T cells was tested to elucidate the role of the DN-TGFβRII in TCR-T cell resistance to TGFβ immunosuppression. Multiple readouts were used to characterize the target-dependent functional engagement of the TCR-T cells and measure their resistance to TGFβ immunosuppression.

a. Process-Similar TCR-T Cell Characterization

First, the cellular composition of process-similar TCR-T cells was characterized by flow cytometery. The following gating strategy was used for the flow characterization of process-similar TCR-T cells: cells were gated from the FSC versus SSC dot plot and singlets were distinguished from the aggregates using FSC-Area versus FSC-Height plot. Viable cells were identified using the Near-Infrared Live-Dead versus FSC-Area plot. Subpopulations were gated from the viable cells and evaluated using the CD34, CD4, CD8 and TGFβRII markers. Expression of the therapeutic TCR was probed by positive detection of MAGEA-A1-derived peptide KVLEYVIKV (SEQ ID NO: 1) bound to HLA-A*02:01 (for TSC-204-A0201) dextramers.

The cellular composition of two batches of DN-TGFβRII-positive and DN-TGFβRII-negative TSC-204-A0201 TCR-T cells were examined by flow cytometry.

Effectors were prepared as described above. After overnight recovery, TCR-T cells were stained with two different panels, either a panel consisting of LIVE/DEAD™ dye, CD4, CD34, TGFβRII, and dextramer, or a panel consisting of live-dead stain, CD4, CD34 and CD8B. Note that product-specific dextramers were used for the staining, using custom-made HLA-A*02:01/KVLEYVIKV (SEQ ID NO: 1) dextramer for TSC-204-A0201 TCR-T cells. Data acquisition was performed on a Cytoflex S. Compensation was performed automatically with the CytExpert software. Data analysis was performed with FlowJo v7.6.5, Excel 2010.

Flow analysis confirmed the expression status of DN-TGFβRII at the surface of the process-similar TCR-T cells engineered with the different delivery vectors and data are shown in Table 14. In particular, the percentage of cells expressing the CD34 tag, which identifies transduced cells, was determined to range from 82% to 88% for both DN-TGFβRII-negative TCR-T cells and DN-TGFβRII-positive TCR-T cells for TSC-204-A0201 TCR-T cells (Table 14). Furthermore, about 70% of transduced (CD34+) DN-TGFβRII-positive TCR-T cells expressed the DN-TGFβRII (Table 14). The gate identifying the DN-TGFβRII-positive cells was set using DN-TGFβRII-negative TCR-T cells as a negative control, thereby confirming that the increased signal for TGFβRII observed in DN-TGFβRII-positive TCR-T cells derives from the expression of exogenous DN-TGFβRII, rather than expression of endogenous TGFβRII.

TABLE 14
TGFβRII expression from flow cytometry data

| | | | | | | RATIO: |
|---|---|---|---|---|---|---|
| | | | | | | (% CD34 + TGFβR |
| | | | % TGFβRII | % TGFβRII | Total | II+)/[Total |
| TSC-204-A0201 | Donor | TCR-T cells product | −CD34+ | +CD34+ | CD34+ (%) | CD34+ (%)] |
| | D5662 | TSC-204-A0201 (+DN-TGFbRII) | 25.90% | 62.80% | 88.7% | 70.80% |
| | | TSC-204-A0201 (noDN-TGFbRII) | 77.40% | 5.37% | 82.8% | 6.49% |
| | D6418 | TSC-204-A0201 (+DN-TGFbRII) | 26.90% | 60.10% | 87.0% | 69.08% |
| | | TSC-204-A0201 (noDN-TGFbRII) | 78.90% | 6.07% | 85.0% | 7.14% |

Altogether, the flow profile of DN-TGFβRII-positive and DN-TGFβRII-negative TCR-T cells were highly similar apart from expression of DN-TGFβRII confirming that process-similar TCR-T cells represent good test articles to functionally study the contribution of DN-TGFβRII to T cell functions.
b. TCR-T Cell Resistance to TGFβ-Mediated Inhibition of Cytokine and Granzyme B Secretion

The ability of the DN-TGFβRII-positive and DN-TGFβRII-negative TCR-T cells to respond to their cognate peptide/MHC by secreting inflammatory cytokines and granzyme B in the presence of TGFβ was tested.

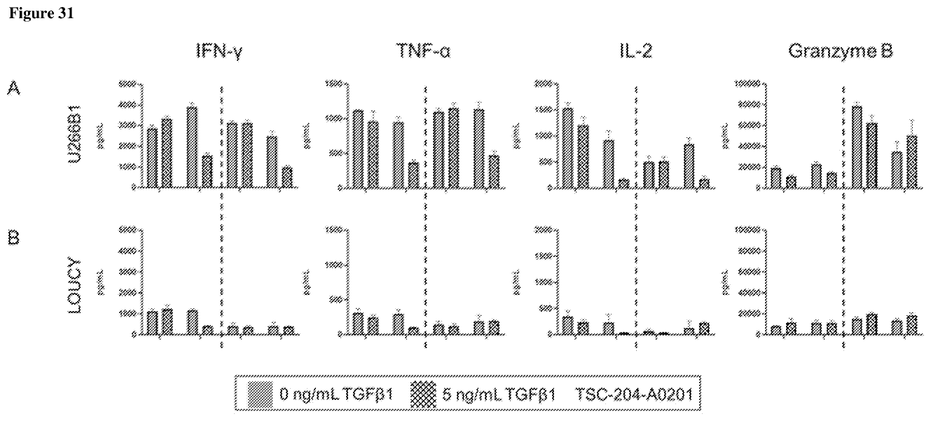

The TCR-T cells tested were pre-incubated for ~20 hours with TGFβ (0 or 5 ng/ml) (Table 13) prior to being incubated for 20 hours with U266B1 target cells (MAGE-A1-positive, HLA-A*02:01-positive cells). At this point, the T cells were spun down, supernatant was completely removed, and a second round of target cells was added. TGFβ was maintained at a concentration of 0 or 5 ng/mL throughout the two rounds of co-culture. Cytokine (IFN-γ, TNF-α, and IL-2) as well as granzyme B secretion were evaluated after the second round of co-culture. Besides U266B1, a negative control cell line (i.e., HLA-A*02:01, MAGE-A1-negative LOUCY cells) was also used in the second round of co-culture. This condition was included to measure the amount of cytokine and Granzyme B produced at the end of the experiment that derived from the first round of stimulation with U266B1 (FIG. 31).

Briefly, serial co-culture assay was used to evaluate resistance to TGFβ-mediated inhibition of cytokine and granzyme B secretion. Specifically, effectors were thawed and allowed to recover overnight as described above. Subsequently, 5E4 live effectors per well were plated in 100 μL cytokine-free T cell medium in a 96 well U-bottom plate and 100 μL of 0 or 10 ng/mL TGFβ1 diluted in T cell medium was added for a final concentration of 0 or 5 ng/mL TGFβ1. After 20-24 hour incubation at 37° C., 5% CO₂, effectors were spun down and the supernatant was carefully discarded without disturbing the cell pellets. Effectors were resuspended in 100 μL of 0 or 10 ng/mL TGFβ1 diluted in T cell medium, and 5E4 live U266B1 cells (MAGE-A1-positive) in 100 μL target cell medium was added for a final TGFβ1 concentration of 0 or 5 ng/ml and an E:T of 1:1. After 20 hour co-culture, effectors were spun down, supernatant was discarded and effectors were resuspended in T cell medium+/−TGFβ1 as described above. A second round of targets, either 5E4 live U266B1 (i.e., MAGE-A1-positive target cell line) or 5E4 live Loucy (i.e., MAGE-A1-negative control cell line) in 100 μL target cell medium was added, and effectors were co-cultured with targets for an additional 20 hours. Subsequently, plates were spun down and supernatants were collected for evaluation of cytokine and granzyme B secretion.

Cytokines and granzyme B secreted into the co-culture supernatants were quantified using an automated ELISA system (ELLA by ProteinSimple) in conjunction with 4-plex (IFN-γ, TNF-α, IL-2 and granzyme B) ELLA cartridges according to instructions of the manufacturer. The raw data were exported as excel files and graphed in GraphPad Prism (v5.02).

In the absence of TGFβ, both DN-TGFβRII positive and DN-TGFβRII negative TCR-T cells displayed a robust target-dependent production of cytokine and of granzyme B, with induction of secretion observed upon co-culture with U266B1 (FIG. 31). Production of cytokine was also observed when the TCR-T cells were co-cultured with LOUCY cells (FIG. 31B). This later observation confirmed that most of the cytokine and of granzyme B measured after two rounds of co-culture with U266B1 derived from the second round of stimulation. Furthermore, when no TGFβ was added to the co-cultures, DN-TGFβRII-positive and DN-TGFβRII-negative TCR-T cells produced comparable levels of cytokine and granzyme B (FIG. 31A).

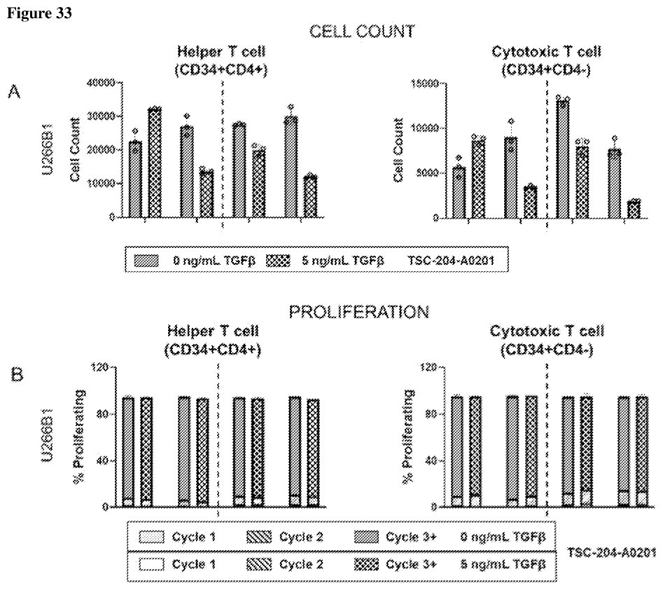

In the presence of physiological level of TGFβ (5 ng/ml), TSC-204-A0201 TCR-T cells lacking DN-TGFβRII showed a 2-3-fold reduction in the total secreted amount of all 3 cytokines (IFN-γ, TNF-α, and IL-2) and a more modest reduction of granzyme B secretion (observed for one of two TSC-204-A0201 batches (FIG. 31A). This observed reduction in cytokine and granzyme B secretion is believed to be due to a combination of TGFβ-mediated transcriptional repression of those genes (Thomas et al. (2005) Cancer Cell 8:369-380), and TGFβ-mediated inhibition of cell expansion (see FIG. 33).

In contrast to DN-TGFβRII-negative TCR-T cells, DN-TGFβRII-positive TSC-204-A0201 TCR-T cells displayed little to no reduction of cytokine and granzyme B secretion (FIG. 31A). It is believed that expression of the DN-TGFβRII shields the TCR-T cells from TGFβ-mediated inhibition of cytokine production. TGFβ did not consistently inhibit granzyme B secretion of DN-TGFβRII-negative TSC-204-A0201 TCR-T cells, possibly due to insufficient depletion of pre-formed granzyme B protein.

Taken together, these data confirmed that expression of the DN-TGFβRII confers resistance to TGFβ-mediated inhibition of target-dependent cytokine and granzyme B secretion.

c. TCR-T Cell Resistance to TGFβ-Mediated Inhibition of T Cell Proliferation

Resistance to TGFβ-mediated inhibition of T cell proliferation was also assessed. TSC-204-A0201 TCR-T cells consist of both helper (CD4⁺) and cytotoxic (CD8⁺) T cells. Unmodified CD4⁺ helper T cells do not naturally express CD8αβ and therefore do not engage type I MHC molecules efficiently if engineered only with a recombinant class-I-restricted TCR. For that reason, TSC-204-A0201 TCR-T cells were engineered to express exogenous CD8 co-receptor (both the CD8α and CD8β chains; see FIG. 30) so that both the engineered helper (CD4⁺) and cytotoxic (CD8⁺) TCR-T cells functionally engage the cognate peptide/MHC and mount a proliferative response.

Resistance to TGFβ-mediated inhibition of proliferation was evaluated in a flow cytometry-based assay. Effectors were thawed and allowed to recover overnight as described above. To eliminate baseline proliferation induced by the T cell cytokines IL-2 and IL-7, effectors were washed once with cytokine-free T cell medium, reseeded in 6-well G-REX® plates at a concentration of 1-2E6 live cell/mL and incubated for an additional 20-24 hours in cytokine-free T cell medium. Adherent target cells were engineered to express the fluorescent protein Nuclight Red. In contrast, non-adherent target cells did not express a fluorescent protein and were therefore labeled with cell trace dye. Effectors were labeled with CellTrace® Violet (CTV) dye, and non-adherent targets (i.e., U266B1 and Loucy) were stained with Far Red CellTrace® dye (FR) as follows: cells were washed once with Easysep and stained for 7 minutes at room temperature with cell trace dye diluted 1:2000 (CTV) or 1:6000 (FR) in EasySep®. Subsequently, target cells were washed twice with target cell medium (Table 13), and effectors were washed twice with cytokine-free T cell medium (Table 13). For co-cultures with non-adherent targets, 5E4 CTV labeled effectors in 100 μL T cell medium containing 0 or 10 ng/mL TGFβ1 were plated in U-bottom plates, and 5E4 FR-labeled target cells in 100 μL target cell medium were added to achieve an effector to target (E:T) ratio of 1:1 and a final TGFβ1 concentration of 0 or 5 ng/mL. For co-cultures with adherent targets (SW1271, HS936T), 5E4 CTV-labeled effectors and TGFβ1 were added to target cells plated the previous day to achieve a final concentration of TGFβ1 of 0 or 5 ng/ml, and an E:T of 1:1. After incubating co-cultures for 3.5 days at 37° C. 5% CO₂, effectors were transferred to a v-bottom plate and stained with the staining reagents listed in Table 13. To enable quantification of absolute T cell numbers, all samples were resuspended in the same volume (100 μL) at the end of the staining procedure and as and additional control, counting beads were added before acquisition.

Data acquisition was performed on a Cytoflex S following SOP-PC-0001-Instrument SOP-Use and Maintenance of the Cytoflex. Compensation was performed with the CytExpert software using single-color controls. Data analysis was performed with FlowJo v7.6.5, Excel 2010.

To enable quantification of absolute T cell numbers, all samples were resuspended in the same volume (100 μL) and were acquired for one minute at the same speed. Both absolute T cell numbers, and % proliferating cells were plotted in Graphpad Prism (v5.02). Gates were drawn to determine the percentage of cells that cycled 1, 2, 3, 4, 5 or 6 times. The percentage of cells that underwent three or more cycles was determined by calculating the sum of cells undergoing 3, 4, 5 and 6 cycles, and the percentage of cells undergoing one, two or three or more cycles was then plotted using GraphPad Prism (v5.02).

Resistance to TGFβ-mediated inhibition of proliferation was assessed in both helper and cytotoxic TCR-T cells and a gating strategy was used.

Target cells and dead cells were labeled with dyes detectable in the APC channel (i.e., NuclightRed for adherent targets; Far Red Cell Trace Dye for non-adherent targets; and Far Red Live Dead Dye to identify dead cells). Therefore, effectors were separated from targets and dead cells by gating on APC-CD3+ events.

Subsequently, transduced (i.e., CD34⁺) T cells were identified in a CD34 versus FSC-Area plot and were further separated into helper T cells (CD4⁺) and cytotoxic T cells (CD4⁻) in a CD3 versus CD4 plot. Since engineered CD4⁺ T cells express exogenous CD8αβ proteins, only the CD4 marker was used to distinguish helper (CD4⁺) and cytotoxic (CD4⁻) TCR-T cells in the flow cytometry analysis. Given that the events analyzed were initially gated on CD3⁺ T cells, the CD4⁻ fraction contained exclusively cytotoxic T cells.

Finally, dilution of the Cell Trace Violet dye was assessed in both the helper and cytotoxic T cell subsets using a CD3 versus CTV plot. TCR-T cells cultured in the absence of target, or in the presence of Loucy (i.e., MAGE-A1 negative target cell line) served as negative controls and were used to identify the position of the CTV peak of undivided cells. Additional gates were drawn to identify cells that had cycled once, twice, or up to 6 times.

To determine whether expression of DN-TGFβRII protected TCR-T cells from TGFβ mediated inhibition of cell expansion and proliferation, CellTrace® Violet (CTV)-labeled TCR-T cells were co-cultured with target cells for 3.5 days at an effector to target ratio of 1:1. TGFβ was added to the co-culture at a final concentration of 0 or 5 ng/ml. At the end of the co-culture, cell number, determined with a flow cytometry-based method as described above, and cell proliferation, assessed by dilution of the CTV dye, were evaluated for transduced helper TCR-T cells (CD34⁺/CD4⁺) and transduced cytotoxic TCR-T cells (CD34⁺/CD4⁻). Furthermore, total percentage of proliferating cells along with the magnitude of the proliferative response were measured by quantifying the percentage of cells that underwent only one or two cycles, versus three or more cell cycles.

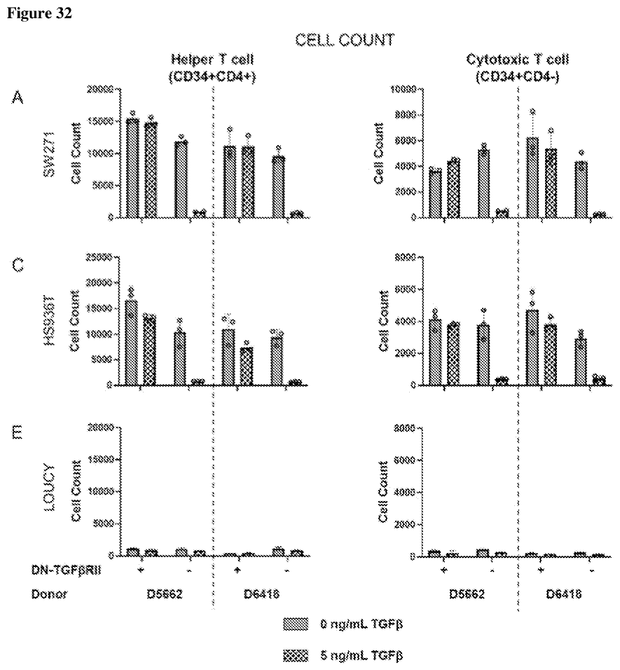

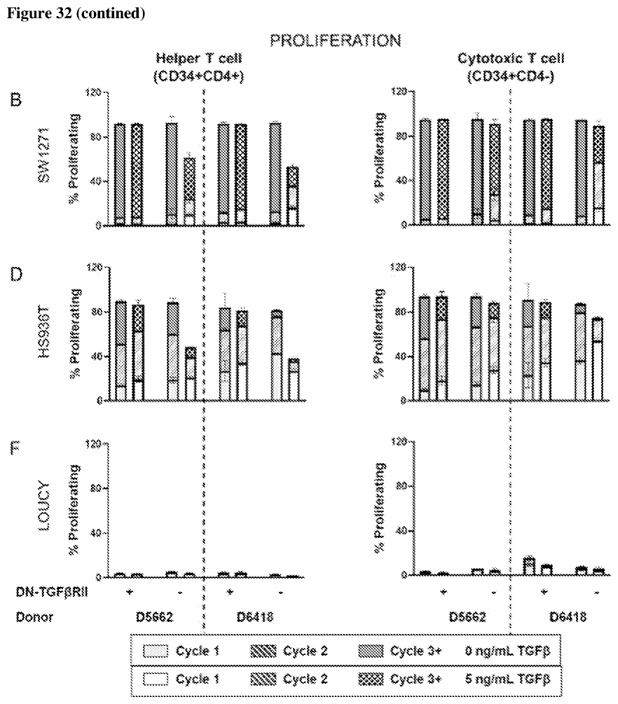

When no TGFβ was added to the co-cultures, both DN-TGFβRII-positive and DN-TGFβRII-negative TSC-204-A0201 TCR-T cells expanded robustly upon stimulation with the MAGE-A1-positive and HLA-A*02:01-positive cell lines SW1271 and HS936T (FIGS. 32A and 32C. Indeed, across two batches of TCR-T cells, both DN-TGFβRII-positive and DN-TGFβRII-negative T cells expanded at least ten-fold compared to TCR-T cells co-cultured with the MAGE-A1-negative cell line Loucy (compare FIG. 32E to FIGS. 32A and 32C. Furthermore, both transduced helper TCR-T cells (CD34⁺CD4⁺) and transduced cytotoxic TCR-T cells (CD34⁺CD4⁻) expanded robustly upon co-culture with these cell lines. SW1271 and HS936T also stimulated robust proliferation of TSC-204-A0201 TCR-T cells and the magnitude of cell proliferation was comparable for DN-TGFβRII-positive and DN-TGFβRII negative TCR-T cells, with most TCR-T cells undergoing 3 or more cycles when co-cultured with SW1271, and the majority of the TCR-T cells undergoing 2 or more cycles upon co-culture with HS936T target cells (FIGS. 32B and 32D).

Addition of TGFβ (5 ng/mL) inhibited the expansion of DN-TGFβRII-negative TSC-204-A0201 TCR-T cells (FIGS. 32A and 32C). This reduction of cell expansion was associated with a sharp reduction in the magnitude of proliferation observed in the live cells at the end of the co-culture. FIGS. 32B and 32D show that for DN-TGFβRII-negative TSC-204-A0201 the total percentage of proliferating cells decreased, and so did the fraction of cells that underwent 3 cycles of more, confirming that TGFβ impedes the T cell proliferative response. Engineered helper T cells appeared to be the most impacted by TGFβ inhibition, although cytotoxic TCR-T cells also seem to present a reduction of cell proliferation.

On the other hand, TGFβ did not inhibit the target dependent expansion and proliferation of DN-TGFβRII-positive TCR-T cells (FIG. 32). For DN-TGFβRII-positive TCR-T cells, the total number of TCR-T cells was comparable whether TGFβ was added to the co-cultures or not, or only slightly reduced, depending on the T cell subset and the target cell line considered. The proportion of cells engaging in cell proliferation was also either not or only slightly influenced by TGFβ exposure. The DN-TGFβRII-positive TCR-T cells proliferated similarly whether they were exposed to TGFβ or not (FIGS. 32B and 32D). Both the cytotoxic and helper T cells making up TSC-204-A0201 were protected from TGFβ-induced inhibition of cell proliferation.

The effect of TGFβ on the expansion and proliferation of TCR-T cells co-cultured with U266B1 was also assessed (Figures igure 33). As observed upon co-culture with the other cell lines, addition of TGFβ to U266B1 co-cultures was associated with a reduction in the total TCR-T cell number. TSC-204-A0201 TCR-T cells also showed a reduction in T cell numbers in the presence of TGFβ without any apparent effect on T cell proliferation (FIGS. 33A and 33B). Since MAGE-A1 is highly expressed in U266B1 when compared to the other cell lines tested, it is possible that U266B1 cells stimulated TCR-T cells more strongly than the other cell lines, enabling TCR-T cells to overcome TGFβ-mediated inhibition of T cell proliferation. The observation that total TCR-T cell numbers were reduced despite minimal inhibition of proliferation may then be explained by an indirect effect of TGFβ on T cell survival. For instance, TGFβ may activate immunosuppressive signaling pathways in U266B1 cells. An indirect mechanism can also explain why DN-TGFβRII-positive TCR-T cells appeared to be somewhat less resistant to the TGFβ-induced reduction in T cell numbers upon co-culture with U266B1, than upon co-culture with the other cell lines (e.g., SW1271, A101D, and HS936T).

Taken together, these data demonstrate that expression of DN-TGFβRII protected TCR-T cells from TGFβ-mediated inhibition of T cell expansion and T cell proliferation. The protective effect of DN-TGFβRII was observed across multiple cancer cell lines. Furthermore, both transduced helper (CD34⁺CD4⁺) and transduced cytotoxic (CD34⁺CD4⁻) T cells were rendered resistant to inhibition by TGFβ.

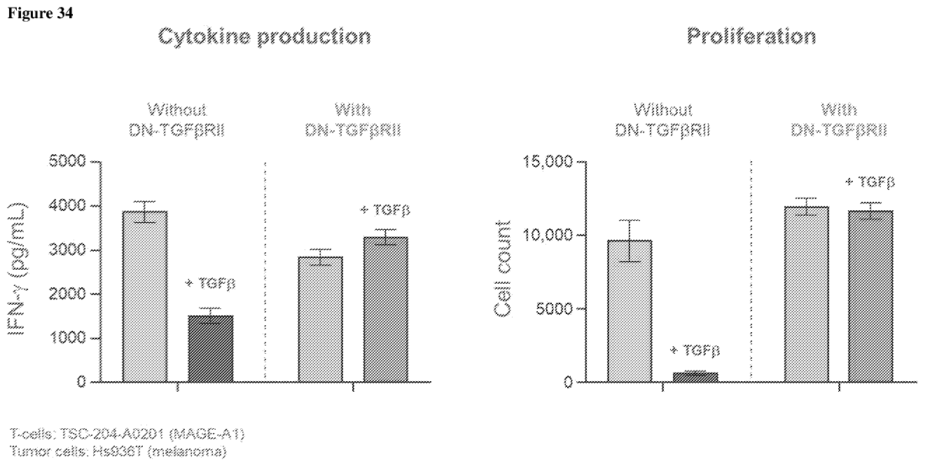

Similar effects of DN-TGFβRII were also observed for TSC-204-A0201 co-cultured with Hs936T (melanoma) cells. FIG. 34 shows expression of dominant-negative TGFβRII (DN-TGFβRII) results in ~2-fold higher cytokine production and ~10-fold higher T-cell proliferation in the presence of TGFβ in vitro.

d. Expression of DN-TGFβII does not Affect TCR-T Cell Cytotoxic Activity

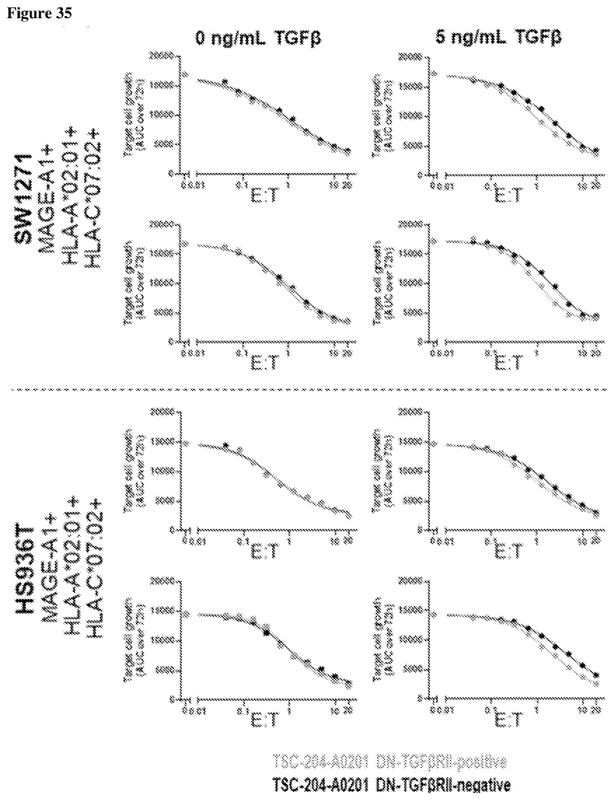

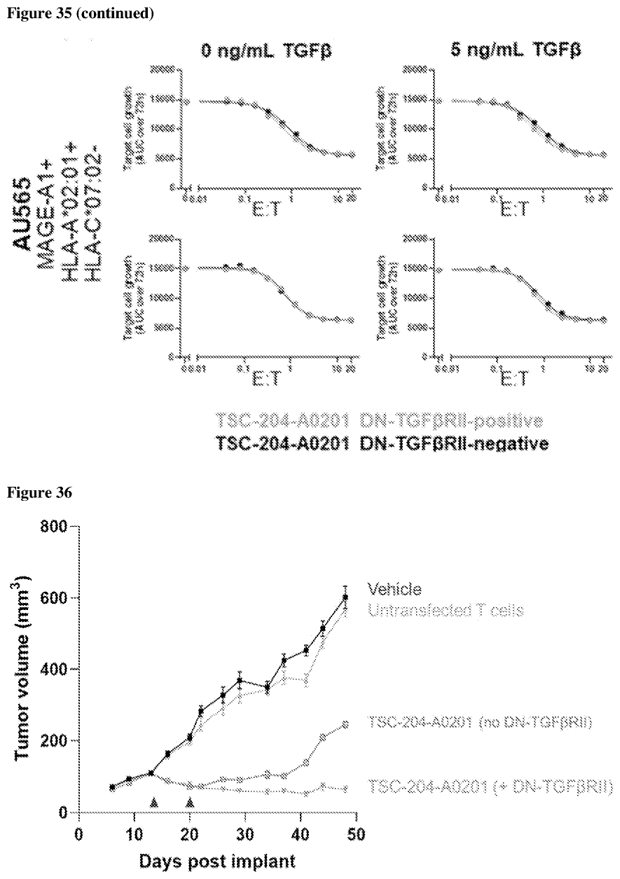

To further test the impact of DN-TGFβRII element on the function of the TCR-T cells, the cytotoxic potential of the two batches of process-similar TSC-204-A0201 expressing or not expressing DN-TGFβRII was tested (FIG. 35). The effector T cells were serially diluted and co-cultured with a fixed number of cancer cell lines to test different effector to target ratios (E:T); TGFβ was added at a final concentration of 0 or 5 ng/mL. MAGE-A1-positive, HLA-A*02:01-positive (i.e., SW1271, AU565 and HS936T) targets were tested. These cells were engineered to express NuclightRed, a fluorescent protein enabling the tracking and quantification of cell growth over time. None of these cell lines appeared to be sensitive to TGFβ.

Resistance to TGFβ-mediated inhibition of killing assay was evaluated with IncuCyte®-based cytotoxicity assay. Specifically, to allow tracking of target cells in the IncuCyte-based cytotoxicity assay, targets were engineered to express the fluorescent protein NuclightRed (NucLightRed, Essen Bioscience) following the manufacturer's instruction. Target cells were plated the day before initiating the co-culture as described above. Effectors were thawed and rested overnight as described above. On the day of co-culture, T cells were harvested, washed, and resuspended in cytokine-free T cell medium or in T cell medium supplemented with TGFβ1 (10 ng/ml). Subsequently, using the corresponding T cell media (+/−TGFβ1, 10 ng/ml) effector cells underwent a serial dilution in a 96-deep well plate to obtain the plating concentrations and 100 μL of effectors were added to the targets, resulting in an E:T titration ranging from 20:1 to 0.04:1 and a final concentration of 0 or 5 ng/ml of TGFβ1. For the target cells only condition, 100 μL of cytokine-free T cell medium or T cell medium supplemented with TGFβ1 (10 ng/ml) was added to the target cells resulting in a final concentration of 0 or 5 ng/ml of TGFβ1. Plates were sealed with Breathable Plate Sealer to limit evaporation of medium and were allowed to settle at room temperature for 10-15 minutes. After an additional 15 minutes incubation at 37° C. 5% CO₂, a Kimwipe was used to wipe off condensation of the bottom of the plates and acquisition was started.

Data acquisition and image analysis were performed on Sartorius IncuCyte® imager and software. The raw data were exported and graphed in GraphPad Prism (v5.02).

In the absence of exogenous TGFβ, TSC-204-A0201 TCR-T cells killed the positive target cell lines (i.e., SW1271, HS936T and AU565) similarly whether the T cells expressed DN-TGFβRII or not (FIG. 35). Specifically, a dose-dependent cytotoxic function was observed with increased killing as the effector to target ratio augmented. The AUC curves representing the target cell growth over 72 hour across the different E:Ts tested overlapped for TSC-204-A0201 TCR-T cells lacking or not DN-TGFβRII, with either batch of material tested (i.e., D5662 and D6418). These data confirm that the DN-TGFβRII element has no influence on the baseline cytotoxic function of the TCR-T cells, regardless of the therapeutic TCR considered.

Moreover, in the presence of physiological concentration of TGFβ (5 ng/ml), the cytotoxic function of TSC-204-A0201 TCR-T cells lacking DN-TGFβRII was only slightly inhibited. The TCR-T cells maintained a potent and selective killing function across the different E:Ts tested, indicating that TGFβ has little influence on the cytotoxic function of the TCR-T cells under the culture conditions of the assay. Indeed, in the presence of TGFβ, the growth curves of the target cells co-cultured with DN-TGFβRII-positive and DN-TGFβRII-negative TCR-T cells were virtually identical: only a modest shift of the AUC-indicating marginally increased killing activity observed with DN-TGFβRII-positive TCR-T cells, likely deriving from the increased proliferation potential of the cells, as described above, was observed.

These date demonstrate that expression of DN-TGFβRII does not influence the baseline functions of TSC-204-A0201 TCR-T cells: target-dependent cytokine production, cell proliferation and cytotoxic function were identical with TCR-T cells expressing or lacking DN-TGFβRII in the absence of TGFβ. TGFβ inhibited target-dependent cytokine and granyme B production of DN-TGFβRII-negative TSC-204-A0201 TCR-T cells and dramatically reduced the expansion of the TCR-T cells in response to their target cells. On the other hand, expression of DN-TGFβRII shielded TSC-204-A0201 TCR-T cells from this TGFβ-mediated inhibition of cytokine secretion and cell expansion. Accordingly, TSC-204-A0201 TCR-T cells are believed to benefit from expression of DN-TGFβRII for long-term persistence in a TGFβ-expressing environment. TGFβ had little effect on the cytotoxic function of the TCR-T cells, whether they expressed DN-TGFβRII or not. This observation indicates that DN-TGFβRII expression does not enhance the risk for tumor lysis syndrome, macrophage activation syndrome, and cytokine release syndrome following administration of TSC-204-A0201 TCR-T cells.

Effects of DN-TGFβRII was also confirmed in vivo. Similarly to Example 11 described above, TSC-204-A0201 cells either expressing or not expressing DN-TGFβRII were tested for their in vivo anti-tumor activity using the xenograft model of U266B1 in female NCG mice described in Example 11. Untransfected (UTF) control T cells and vehicle (PBS) treatments were included as controls.

Briefly, process-similar TSC-204-A0201 TCR-T cells expressing or not DN-TGFβRII (comparable to those described above) and UTF control T cells were engineered from the same donor. As described in Example 11 above, once tumor engraftment was successful (tumors reaching 100 mm³ on average), animals received two doses, 7 days apart, of TSC-204-A0201 expressing DN-TGFβRII, of TSC-204-A0201 lacking DN-TGFβRII, of UTF T cells, or of vehicle (PBS). Anti-tumor efficacy was evaluated by biweekly tumor volume measurements.

As described in Example 11, tumors in animals receiving UTF control T cells or vehicle (PBS) grew similarly, reaching ~600 mm³ on average by day 48 post tumor inoculation. TSC-204-A0201 expressing DN-TGFβRII presented a robust anti-tumor activity as demonstrated by tumor volumes remaining at an average of ~60 mm³ by day 48 post-tumor inoculation. TCR-T cells lacking DN-TGFβRII presented an initial anti-tumor activity comparable to the TCR-T cells expressing DN-TGFβRII, but tumors resumed growth in these experiments two weeks after the injection of TCR-T cells; tumors eventually reached ~245 mm³ on average by day 48 post-tumor inoculation. Together with the in vitro data presented above, FIG. 36 shows that expression of dominant-negative TGFβRII (DN-TGFβRII) results in durable responses of tumors in vivo.

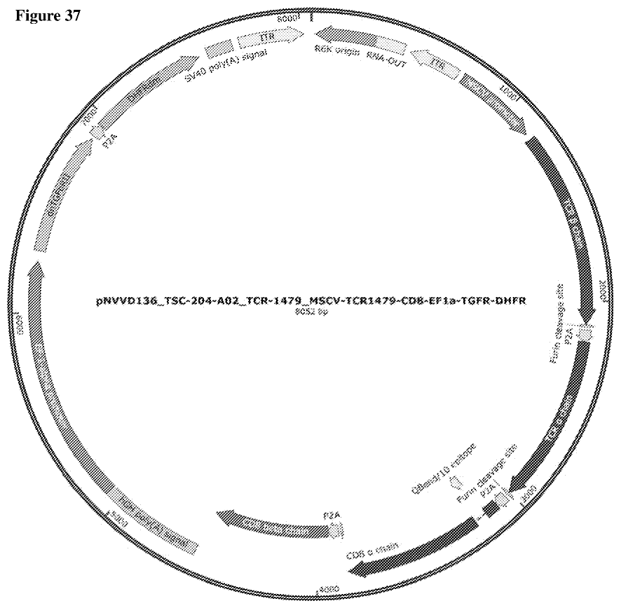

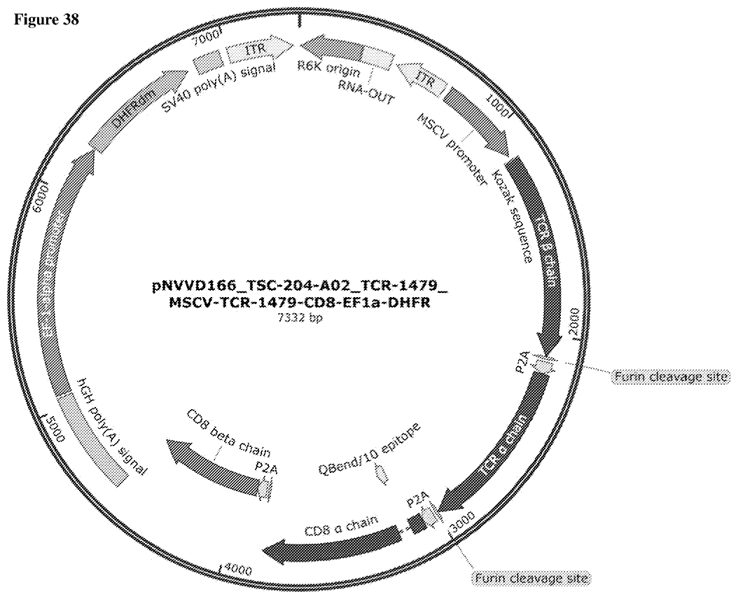

Vector pNVVD136 was used in this study as the vector for TSC-204-A0201 with DN-TGFβRII. The full sequence of the vector pNVVD136 is shown in Table 3 and vector map is shown in FIG. 37. Vector pNVVD166 was used in this study as the vector for TSC-204-A0201 without DN-TGFβRII. The full sequence of the vector pNVVD166 is shown in Table 3 and vector map is shown in FIG. 38.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the World Wide Web at tigr.org and/or the National Center for Biotechnology Information (NCBI) on the World Wide Web at ncbi.nlm.nih.gov.

### EQUIVALENTS AND SCOPE

The details of one or more embodiments encompassed by the present invention are set forth in the description above. Although representative, exemplary materials and methods have been described above, any materials and methods similar or equivalent to those described herein may be used in the practice or testing of embodiments encompassed by the present invention. Other features, objects and advantages related to the present invention are apparent from the description. Unless defined otherwise, all technical and scientific terms used herein have the same meaning as commonly understood by one of ordinary skill in the art to which the present invention belongs. In the case of conflict, the present description provided above will control.

Those skilled in the art will recognize or be able to ascertain using no more than routine experimentation, many equivalents to the specific embodiments encompassed by the present invention described herein. The scope encompassed by the present invention is not intended to be limited to the description provided herein and such equivalents are intended to be encompassed by the appended claims.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges may assume any specific value or subrange within the stated ranges in different embodiments encompassed by the present invention, to the tenth of the unit of the lower limit of the range, unless the context clearly dictates otherwise.

In addition, it is to be understood that any particular embodiment encompassed by the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions encompassed by the present invention (e.g., any antibiotic, therapeutic or active ingredient; any method of production; any method of use; etc.) may be excluded from any one or more claims, for any reason, whether or not related to the existence of prior art.

It is to be understood that the words which have been used are words of description rather than limitation, and that changes may be made within the purview of the appended claims without departing from the true scope and spirit encompassed by the present invention in its broader aspects.

While the present invention has been described at some length and with some particularity with respect to several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope encompassed by the present invention.

1.-55. (canceled)

56. A binding protein that binds a polypeptide comprising an immunogenic peptide comprising or consisting of a peptide epitope selected from VLEYVIKV (SEQ ID NO: 2), KVLEYVIK (SEQ ID NO: 3), and peptide sequences listed in Table 1, optionally wherein the binding protein:
1) is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain;
2) comprises:
a) a TCR alpha chain variable (V_{α}) domain comprising CDR1 having at least about 80% identity to residues 48-53 of SEQ ID NO: 9 (VSGLRG), CDR2 having at least about 80% identity to residues 71-77 of SEQ ID NO: 9 (LYSAGEE), and CDR3 having at least about 80% identity to residues 109-119 of SEQ ID NO: 9 (CAVSYGONFVF); and
b) a TCR beta chain variable (V_{β}) domain comprising CDR1 having at least about 80% identity to residues 46-50 of SEQ ID NO: 11 (SGHTS), CDR2 having at least about 80% identity to residues 68-73 of SEQ ID NO: 11 (YDEGEE), and CDR3 having at least about 80% identity to residues 110-123 of SEQ ID NO: 11 (CASSLGQLNTEAFF);
3) comprises:
a) a TCR alpha chain variable (V_{α}) domain with at least about 80% identity to residues 1-130 of SEQ ID NO: 9; and
b) a TCR beta chain variable (V_{β}) domain with at least about 80% identity to residues 1-133 of SEQ ID NO: 11;
4) comprises:
a) a TCR alpha chain with at least about 80% identity to the sequence of SEQ ID NO: 9; and
b) a TCR beta chain with at least about 80% identity to the sequence of SEQ ID NO: 11;
5) comprises:
a) a T cell receptor (TCR) alpha chain CDR sequence with at least about 80% identity to a TCR alpha chain CDR sequence selected from the group consisting of TCR alpha chain CDR sequences listed in Table 2; and/or
b) a TCR beta chain CDR sequence with at least about 80% identity to a TCR beta chain CDR sequence selected from the group consisting of TCR beta chain CDR sequences listed in Table 2, wherein the binding protein is capable of binding to a MAGEA1 immunogenic peptide-MHC (pMHC) complex, optionally wherein the binding affinity has a K_{d} less than or equal to about 5×10⁻⁴ M;
6) comprises:
a) a TCR alpha chain variable (V_{α}) domain sequence with at least about 80% identity to a TCR V_{α} domain sequence selected from the group consisting of TCR V_{α} domain sequences listed in Table 2; and/or
b) a TCR beta chain variable (V_{β}) domain sequence with at least about 80% identity to a TCR V_{β} domain sequence selected from the group consisting of TCR V_{β} domain sequences listed in Table 2, wherein the binding protein is capable of binding to a MAGEA1 immunogenic peptide-MHC (pMHC) complex, optionally wherein the binding affinity has a K_{d} less than or equal to about 5×10⁻⁴ M;
7) comprises:
a) a TCR alpha chain sequence with at least about 80% identity to a TCR alpha chain sequence selected from the group consisting of TCR alpha chain sequences listed in Table 2; and/or
b) a TCR beta chain sequence with at least about 80% identity to a TCR beta chain sequence selected from the group consisting of TCR beta chain sequences listed in Table 2, wherein the binding protein is capable of binding to a MAGEA1 immunogenic peptide-MHC (pMHC) complex, optionally wherein the binding affinity has a K_{d} less than or equal to about 5×10⁻⁴ M;
8) comprises:
a) a TCR alpha chain CDR sequence selected from the group consisting of TCR alpha chain CDR sequences listed in Table 2; and/or
b) a TCR beta chain CDR sequence selected from the group consisting of TCR beta chain CDR sequences listed in Table 2, wherein the binding protein is capable of binding to a MAGEA1 immunogenic peptide-MHC (pMHC) complex, optionally wherein the binding affinity has a K_{d} less than or equal to about 5×10⁻⁴ M;
9) comprises:
a) a TCR alpha chain variable (V_{α}) domain sequence selected from the group consisting of TCR V_{α} domain sequences listed in Table 2; and/or
b) a TCR beta chain variable (V_{β}) domain sequence selected from the group consisting of TCR V_{β} domain sequences listed in Table 2, wherein the binding protein is capable of binding to a MAGEA1 immunogenic peptide-MHC (pMHC) complex, optionally wherein the binding affinity has a K_{d} less than or equal to about 5×10⁻⁴ M; and/or
10) comprises:
a) a TCR alpha chain sequence selected from the group consisting of TCR alpha chain sequences listed in Table 2; and/or
b) a TCR beta chain sequence selected from the group consisting of TCR beta chain sequences listed in Table 2, wherein the binding protein is capable of binding to a MAGEA1 immunogenic peptide-MHC (pMHC) complex, optionally wherein the binding affinity has a K_{d} less than or equal to about 5×10⁻⁴ M.

57.-62. (canceled)

63. The binding protein of claim 56, wherein 1) the TCR alpha chain CDR, TCR V_{α} domain, and/or TCR alpha chain is encoded by a TRAV, TRAJ, and/or TRAC gene or fragment thereof selected from the group of TRAV, TRAJ, and TRAC genes listed in Table 2, and/or 2) the TCR beta chain CDR, TCR V_{β} domain, and/or TCR beta chain is encoded by a TRBV, TRBJ, and/or TRBC gene or fragment thereof selected from the group of TRBV, TRBJ, and TRBC genes listed in Table 2, and/or 3) each CDR of the binding protein has up to five amino acid substitutions, insertions, deletions, or a combination thereof as compared to the cognate reference CDR sequence listed in Table 2.

64. The binding protein of claim 56, wherein:
1) the binding protein is chimeric, humanized, or human;
2) the binding protein comprises a binding domain having a transmembrane domain, and an effector domain that is intracellular;
3) the TCR alpha chain and the TCR beta chain are covalently linked, optionally wherein the TCR alpha chain and the TCR beta chain are covalently linked through a linker peptide;
4) the TCR alpha chain and/or the TCR beta chain are covalently linked to a moiety, optionally wherein the covalently linked moiety
a) comprises an affinity tag or a label, optionally wherein the affinity tag is selected from the group consisting of a CD34 enrichment tag, glutathione-S-transferase (GST), calmodulin binding protein (CBP), protein C tag, Myc tag, HaloTag, HA tag, Flag tag, His tag, biotin tag, and V5 tag, and/or wherein the label is a fluorescent protein; and/or
b) is selected from the group consisting of an inflammatory agent, cytokine, toxin, cytotoxic molecule, radioactive isotope, or antibody or antigen-binding fragment thereof;
5) the binding protein binds to the pMHC complex on a cell surface, optionally wherein the MHC-peptide complex comprises the immunogenic peptide in the context of an MHC molecule, further optionally wherein
i) the MHC molecule
a) is an MHC multimer, optionally wherein the MHC multimer is a tetramer;
b) is an MHC class I molecule; and/or
c) comprises an MHC alpha chain that is an HLA serotype selected from the group consisting of HLA-A*02, HLA-A*03, HLA-A*01, HLA-A*11, HLA-A*24, HLA-B*07, HLA-C*07, HLA-C*01, HLA-C*02, HLA-C*03, HLA-C*04, HLA-C*05, HLA-C*06, HLA-C*08, HLA-C*12, HLA-C*14, HLA-C*15, HLA-C*16, HLA-C*17, and HLA-C*18, optionally wherein the HLA allele is selected from the group consisting of HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:12, HLA-A*02:13, HLA-A*02:14, HLA-A*02:16, HLA-A*02:17, HLA-A*02:19, HLA-A*02:20, HLA-A*02:22, HLA-A*02:24, HLA-A*02:30, HLA-A*02:42, HLA-A*02:53, HLA-A*02:60, HLA-A*02:74 allele, HLA-A*03:01, HLA-A*03:02, HLA-A*03:05, HLA-A*03:07, HLA-A*01:01, HLA-A*01:02, HLA-A*01:03, HLA-A*01:16 allele, HLA-A*11:01, HLA-A*11:02, HLA-A*11:03, HLA-A*11:04, HLA-A*11:05, HLA-A*11:19 allele, HLA-A*24:02, HLA-A*24:03, HLA-A*24:05, HLA-A*24:07, HLA-A*24:08, HLA-A*24:10, HLA-A*24:14, HLA-A*24:17, HLA-A*24:20, HLA-A*24:22, HLA-A*24:25, HLA-A*24:26, HLA-A*24:58 allele, HLA-B*07:02, HLA-B*07:04, HLA-B*07:05, HLA-B*07:09, HLA-B*07:10, HLA-B*07:15, HLA-B*07:21, HLA-C*07:02, HLA-C*07:01, HLA-C*04:01, HLA-C*06:02, HLA-C*03:04, HLA-C*05:01, HLA-C*16:01, HLA-C*02:02, HLA-C*03:03, HLA-C*12:03, HLA-C*08:02, HLA-C*01:02, HLA-C*17:01, HLA-C*15:02, HLA-C*14:02, HLA-C*12:02, HLA-C*07:04, HLA-C*08:01, HLA-C*03:02, HLA-C*18:01, HLA-C*15:05, HLA-C*16:02, HLA-C*08:04, HLA-C*03:05, and HLA-C*14:03 allele; optionally wherein the HLA serotype is HLA-A*02; and further optionally wherein the HLA-A*02 is HLA-A*02:01;
ii) the peptide epitope and the MHC molecule are covalently linked and/or wherein the alpha and beta chains of the MHC molecule are covalently linked; and/or
iii) the MHC-peptide complex comprises a detectable label, optionally wherein the detectable label is a fluorophore;
6) binding of the binding protein to the MAGEA1 peptide-MHC (pMHC) complex elicits an immune response, optionally wherein the immune response is
a) a T cell response and/or a CD8+ T cell response and/or
b) selected from the group consisting of T cell expansion, cytokine release, and/or cytotoxic killing;
7) the binding protein is capable of specifically and/or selectively binding to the MAGEA1 immunogenic peptide-MHC (pMHC) complex with a K_{d} less than or equal to about 1×10⁻⁴ M, less than or equal to about 5×10⁻⁵ M, less than or equal to about 1×10⁻⁵ M, less than or equal to about 5×10⁻⁶ M, less than or equal to about 1×10⁻⁶ M, less than or equal to about 5×10⁻⁷ M, less than or equal to about 1×10⁻⁷ M, less than or equal to about 5×10⁻⁸ M, less than or equal to about 1×10⁻⁸ M, less than or equal to about 5×10⁻⁹ M, less than or equal to about 1×10⁻⁹ M, less than or equal to about 5×10⁻¹⁰ M, less than or equal to about 1×10⁻¹⁰ M, less than or equal to about 5×10⁻¹¹ M, less than or equal to about 1×10⁻¹¹ M, less than or equal to about 5×10⁻¹² M, or less than or equal to about 1×10⁻¹² M;
8) the binding protein has a higher binding affinity to the peptide-MHC (pMHC) than does a known T-cell receptor, optionally wherein the higher binding affinity is at least 1.05-fold higher;
9) the binding protein induces higher T cell expansion, cytokine release, and/or cytotoxic killing than does a known T-cell receptor when contacted with target cells with a heterozygous expression of MAGEA1,
optionally wherein the induction is at least 1.05-fold higher, optionally wherein the cytotoxic killing is killing of a target cancer cell,
further optionally wherein the cancer is selected from the group consisting of melanoma, head & neck cancer, lung cancer, cervical cancer, hepatocellular carcinoma, colorectal cancer, gastrointestinal cancer, colorectal cancer, gastrointestinal cancer, breast invasive carcinoma, and bladder urothelial carcinoma; and/or
10) the binding protein does not bind to a pMHC complex comprising a PIEZO1, NBEAL1, NBEAL2, and/or EPN2 peptide epitope.

65.-78. (canceled)

79. A TCR alpha chain and/or beta chain selected from the group consisting of TCR alpha chain and beta chain sequences listed in Table 2.

80. An isolated nucleic acid molecule i) that hybridizes, under stringent conditions, with the complement of a nucleic acid encoding a polypeptide selected from the group consisting of polypeptide sequences listed in Table 2, ii) a sequence with at least about 80% homology to a nucleic acid encoding a polypeptide selected from the group consisting of the polypeptide sequences listed in Table 2, and/or iii) ii) a sequence with at least about 80% homology to a nucleic acid encoding listed in Table 2, optionally wherein the isolated nucleic acid molecule comprises 1) a TRAV, TRAJ, and/or TRAC gene or fragment thereof selected from the group of TRAV, TRAJ, and TRAC genes listed in Table 2 and/or 2) a TRBV, TRBJ, and/or TRBC gene or fragment thereof selected from the group of TRBV, TRBJ, and TRBC genes listed in Table 2; further optionally wherein the nucleic acid is codon optimized for expression in a host cell.

81. (canceled)

82. A vector comprising the isolated nucleic acid of claim 80, optionally wherein:
i) the vector is a cloning vector, expression vector, or viral vector;
ii) the vector comprises a vector sequence listed in Table 3; and/or
iii) the vector further comprises a nucleic acid sequence encoding CD8α, CD8β, a dominant negative TGFβ receptor II (DN-TGFβRII), selectable protein marker,
optionally wherein
a) the selectable protein marker is dihydrofolate reductase (DHFR);
b) the nucleic acid sequence encoding CD8α, CD8β, the DN-TGFβRII, and/or the selectable protein marker is operably linked to a nucleic acid encoding a tag,
optionally wherein
1) the nucleic acid encoding a tag is at the 5′ upstream of the nucleic acid sequence encoding CD8α, CD8β, the DN-TGFβRII, and/or the selectable protein marker such that the tag is fused to the N-terminus of CD8α, CD8β, the DN-TGFβRII, and/or the selectable protein marker; and/or
2) the tag is a CD34 enrichment tag; and/or
c) the nucleic acid sequence encoding TCRα, TCRβ, CD8α, CD8β, the DN-TGFβRII, and/or the selectable protein marker are interconnected with an internal ribosome entry site or a nucleic acid sequence encoding a self-cleaving peptide, optionally wherein the self-cleaving peptide is P2A, E2A, F2A or T2A.

83.-88. (canceled)

89. A host cell which comprises the isolated nucleic acid encoding the binding protein, comprises the vector comprising an isolated nucleic acid encoding the binding protein, and/or expresses the binding protein, according to claim 56, optionally wherein the cell is genetically engineered.

90. The host cell of claim 89, wherein the host cell has one or more of the following properties:
1) comprises a chromosomal gene knockout of a TCR gene, an HLA gene, or both;
2) comprises a knockout of an HLA gene selected from an α1 macroglobulin gene, α2 macroglobulin gene, α3 macroglobulin gene, β1 microglobulin gene, β2 microglobulin gene, and combinations thereof;
3) comprises a knockout of a TCR gene selected from a TCR α variable region gene, TCR β variable region gene, TCR constant region gene, and combinations thereof;
4) expresses CD8α, CD8β, a DN-TGFβRII, and/or a selectable protein marker, optionally wherein the selectable protein marker is DHFR, further optionally wherein the CD8α, CD8β, the DN-TGFβRII, and/or the selectable protein marker is fused to a CD34 enrichment tag, optionally wherein host cells are enriched using the CD34 enrichment tag;
5) is a hematopoietic progenitor cell, peripheral blood mononuclear cell (PBMC), cord blood cell, or immune cell,
optionally wherein the immune cell is a T cell, cytotoxic lymphocyte, cytotoxic lymphocyte precursor cell, cytotoxic lymphocyte progenitor cell, cytotoxic lymphocyte stem cell, CD4⁺ T cell, CD8⁺ T cell, CD4/CD8 double negative T cell, gamma delta (γδ) T cell, natural killer (NK) cell, NK-T cell, dendritic cell, or a combination thereof,
further optionally wherein the T cell is
a) a naive T cell, central memory T cell, effector memory T cell, or a combination thereof;
b) a primary T cell or a cell of a T cell line; and/or
c) does not express or has a lower surface expression of an endogenous TCR;
6) is capable of producing a cytokine or a cytotoxic molecule when contacted with a target cell that comprises a peptide-MHC (pMHC) complex comprising a MAGEA1 peptide epitope in the context of an MHC molecule,
optionally wherein
a) the host cell is contacted with the target cell in vitro, ex vivo, or in vivo;
b) the cytokine is TNF-α, IL-2, and/or IFN-γ;
c) the cytotoxic molecule is perforins and/or granzymes, optionally wherein the cytotoxic molecule is granzyme B;
d) the host cell is capable of producing a higher level of cytokine or a cytotoxic molecule when contacted with a target cell with a heterozygous expression of MAGEA1, optionally wherein the host cell is capable of producing an at least 1.05-fold higher level of cytokine or a cytotoxic molecule;
e) the target cell is cell line or a primary cell, optionally wherein the target cell is selected from the group consisting of a HEK293 derived cell line, a cancer cell line, a primary cancer cell, a transformed cell line, and an immortalized cell line; and/or
f) the MAGEA1 immunogenic peptide comprises or consists of a peptide epitope selected from VLEYVIKV (SEQ ID NO: 2), KVLEYVIK (SEQ ID NO: 3), and peptide sequences listed in Table 1 and/or wherein the MHC-peptide complex
comprises the immunogenic peptide in the context of an MHC molecule, further optionally wherein
i) the MHC molecule
a) is an MHC multimer, optionally wherein the MHC multimer is a tetramer;
b) is an MHC class I molecule; and/or
c) comprises an MHC alpha chain that is an HLA serotype selected from the group consisting of HLA-A*02, HLA-A*03, HLA-A*01, HLA-A*11, HLA-A*24, HLA-B*07, HLA-C*07, HLA-C*01, HLA-C*02, HLA-C*03, HLA-C*04, HLA-C*05, HLA-C*06, HLA-C*08, HLA-C*12, HLA-C*14, HLA-C*15, HLA-C*16, HLA-C*17, and HLA-C*18, optionally wherein the HLA allele is selected from the group consisting of HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:12, HLA-A*02:13, HLA-A*02:14, HLA-A*02:16, HLA-A*02:17, HLA-A*02:19, HLA-A*02:20, HLA-A*02:22, HLA-A*02:24, HLA-A*02:30, HLA-A*02:42, HLA-A*02:53, HLA-A*02:60, HLA-A*02:74 allele, HLA-A*03:01, HLA-A*03:02, HLA-A*03:05, HLA-A*03:07, HLA-A*01:01, HLA-A*01:02, HLA-A*01:03, HLA-A*01:16 allele, HLA-A*11:01, HLA-A*11:02, HLA-A*11:03, HLA-A*11:04, HLA-A*11:05, HLA-A*11:19 allele, HLA-A*24:02, HLA-A*24:03, HLA-A*24:05, HLA-A*24:07, HLA-A*24:08, HLA-A*24:10, HLA-A*24:14, HLA-A*24:17, HLA-A*24:20, HLA-A*24:22, HLA-A*24:25, HLA-A*24:26, HLA-A*24:58 allele, HLA-B*07:02, HLA-B*07:04, HLA-B*07:05, HLA-B*07:09, HLA-B*07:10, HLA-B*07:15, HLA-B*07:21, HLA-C*07:02, HLA-C*07:01, HLA-C*04:01, HLA-C*06:02, HLA-C*03:04, HLA-C*05:01, HLA-C*16:01, HLA-C*02:02, HLA-C*03:03, HLA-C*12:03, HLA-C*08:02, HLA-C*01:02, HLA-C*17:01, HLA-C*15:02, HLA-C*14:02, HLA-C*12:02, HLA-C*07:04, HLA-C*08:01, HLA-C*03:02, HLA-C*18:01, HLA-C*15:05, HLA-C*16:02, HLA-C*08:04, HLA-C*03:05, and HLA-C*14:03 allele; optionally wherein the HLA serotype is HLA-A*02; and further optionally wherein the HLA-A*02 is HLA-A*02:01;
ii) the peptide epitope and the MHC molecule are covalently linked and/or wherein the alpha and beta chains of the MHC molecule are covalently linked; and/or
iii) the MHC-peptide complex comprises a detectable label, optionally wherein the detectable label is a fluorophore;
7) is capable of killing a target cell that comprises a peptide-MHC (pMHC) complex comprising the MAGEA1 peptide epitope in the context of an MHC molecule,
optionally wherein
a) the killing is determined by a killing assay;
b) the ratio of the host cell and the target cell in the killing assay is from 20:1 to 1:4;
c) the target cell is a target cell pulsed with 1 μg/mL to 50 μg/mL of MAGEA1 peptide, optionally wherein the target cell is a cell monoallelic for an MHC matched to the MAGEA1 peptide;
d) the host cell is capable of killing a higher number of target cells when contacted with target cells with a heterozygous expression of MAGEA1, optionally wherein the cell killing is at least 1.05-fold higher;
e) the target cell is cell line or a primary cell, optionally wherein the target cell is selected from the group consisting of a HEK293 derived cell line, a cancer cell line, a primary cancer cell, a transformed cell line, and an immortalized cell line;
f) the MAGEA1 immunogenic peptide comprises or consists of a peptide epitope selected from VLEYVIKV (SEQ ID NO: 2), KVLEYVIK (SEQ ID NO: 3), and peptide sequences listed in Table 1 and/or wherein the MHC-peptide complex comprises the immunogenic peptide in the context of an MHC molecule, further optionally wherein
i) the MHC molecule
a) is an MHC multimer, optionally wherein the MHC multimer is a tetramer;
b) is an MHC class I molecule; and/or
c) comprises an MHC alpha chain that is an HLA serotype selected from the group consisting of HLA-A*02, HLA-A*03, HLA-A*01, HLA-A*11, HLA-A*24, HLA-B*07, HLA-C*07, HLA-C*01, HLA-C*02, HLA-C*03, HLA-C*04, HLA-C*05, HLA-C*06, HLA-C*08, HLA-C*12, HLA-C*14, HLA-C*15, HLA-C*16, HLA-C*17, and HLA-C*18, optionally wherein the HLA allele is selected from the group consisting of HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:12, HLA-A*02:13, HLA-A*02:14, HLA-A*02:16, HLA-A*02:17, HLA-A*02:19, HLA-A*02:20, HLA-A*02:22, HLA-A*02:24, HLA-A*02:30, HLA-A*02:42, HLA-A*02:53, HLA-A*02:60, HLA-A*02:74 allele, HLA-A*03:01, HLA-A*03:02, HLA-A*03:05, HLA-A*03:07, HLA-A*01:01, HLA-A*01:02, HLA-A*01:03, HLA-A*01:16 allele, HLA-A*11:01, HLA-A*11:02, HLA-A*11:03, HLA-A*11:04, HLA-A*11:05, HLA-A*11:19 allele, HLA-A*24:02, HLA-A*24:03, HLA-A*24:05, HLA-A*24:07, HLA-A*24:08, HLA-A*24:10, HLA-A*24:14, HLA-A*24:17, HLA-A*24:20, HLA-A*24:22, HLA-A*24:25, HLA-A*24:26, HLA-A*24:58 allele, HLA-B*07:02, HLA-B*07:04, HLA-B*07:05, HLA-B*07:09, HLA-B*07:10, HLA-B*07:15, HLA-B*07:21, HLA-C*07:02, HLA-C*07:01, HLA-C*04:01, HLA-C*06:02, HLA-C*03:04, HLA-C*05:01, HLA-C*16:01, HLA-C*02:02, HLA-C*03:03, HLA-C*12:03, HLA-C*08:02, HLA-C*01:02, HLA-C*17:01, HLA-C*15:02, HLA-C*14:02, HLA-C*12:02, HLA-C*07:04, HLA-C*08:01, HLA-C*03:02, HLA-C*18:01, HLA-C*15:05, HLA-C*16:02, HLA-C*08:04, HLA-C*03:05, and HLA-C*14:03 allele; optionally wherein the HLA serotype is HLA-A*02; and further optionally wherein the HLA-A*02 is HLA-A*02:01;
ii) the peptide epitope and the MHC molecule are covalently linked and/or wherein the alpha and beta chains of the MHC molecule are covalently linked; and/or
iii) the MHC-peptide complex comprises a detectable label, optionally wherein the detectable label is a fluorophore;
8) does not induce T cell expansion, cytokine release, or cytotoxic killing when contacted with a target cell that comprises a peptide-MHC (pMHC) complex comprising a PIEZO1, NBEAL1, NBEAL2, and/or EPN2 peptide epitope;
9) does not express MAGEA1 antigen, is not recognized by a binding protein of claim 56, is not of serotype HLA-A*02, and/or does not express an HLA-A*02 allele.

91.-114. (canceled)

115. A population of host cells according to claim 89.

116. A composition comprising a) a binding protein, b) an isolated nucleic acid encoding the binding protein, c) a vector comprising an isolated nucleic acid encoding the binding protein, d) a host cell that expresses the binding protein, and/or e) a population of host cells that expresses the binding protein, according to claim 56, and a carrier.

117. A device or kit comprising a) a binding protein, b) an isolated nucleic acid encoding the binding protein, c) a vector comprising an isolated nucleic acid encoding the binding protein, d) a host cell that expresses the binding protein, and/or e) a population of host cells that expresses the binding protein, according to claim 56, said device or kit optionally comprising a reagent to detect binding of a), d) and/or e) to a pMHC complex.

118. A method of producing a binding protein according to claim 56, wherein the method comprises the steps of: (i) culturing a transformed host cell which has been transformed by a nucleic acid comprising a sequence encoding a binding protein according to claim 56 under conditions suitable to allow expression of said binding protein; and (ii) recovering the expressed binding protein.

119. A method of producing a host cell expressing a binding protein according to claim 56, wherein the method comprises the steps of: (i) introducing a nucleic acid comprising a sequence encoding a binding protein according to claim 56 into the host cell; and (ii) culturing the transformed host cell under conditions suitable to allow expression of said binding protein.

120. A method of detecting the presence or absence of a MAGEA1 antigen and/or a cell expressing MAGEA1, optionally wherein the cell is a hyperproliferative cell, comprising detecting the presence or absence of said MAGEA1 antigen in a sample by use of at least one binding protein according to, at least one host cell that expresses the binding protein, or a population of host cells that express the binding protein, according to claim 56, wherein detection of the MAGEA1 antigen is indicative of the presence of a MAGEA1 antigen and/or cell expressing MAGEA1, optionally wherein
1) the at least one binding protein, or the at least one host cell, forms a complex with the MAGEA1 peptide in the context of an MHC molecule, and the complex is detected in the form of fluorescence activated cell sorting (FACS), enzyme linked immunosorbent assay (ELISA), radioimmune assay (RIA), immunochemically, Western blot, or intracellular flow assay; and/or
2) the method further comprises obtaining the sample from a subject.

121.-122. (canceled)

123. A method of detecting the level of a disorder characterized by MAGEA1 expression in a subject, comprising:
a) contacting a sample obtained from the subject with at least one binding protein, at least one host cell that expresses the binding protein, or a population of host cells that express the binding protein, according to claim 56; and
b) detecting the level of reactivity,
wherein the presence or a higher level of reactivity compared to a control level indicates the level of the disorder characterized by MAGEA1 expression in the subject;
optionally wherein
1) the control level is
A) a reference number; and/or
B) a level from a subject without the disorder characterized by MAGEA1 expression;
2) the level of reactivity is indicated by
A) the presence of binding and/or
B) T cell activation and/or effector function, optionally wherein the T cell activation or effector function is T cell proliferation, killing, or cytokine release; and/or
3) the T cell binding, activation, and/or effector function is detected using fluorescence activated cell sorting (FACS), enzyme linked immunosorbent assay (ELISA), radioimmune assay (RIA), immunochemically, Western blot, or intracellular flow assay.

124.-125. (canceled)

126. A method for monitoring the progression of a disorder characterized by MAGEA1 expression in a subject, the method comprising:
a) detecting in a subject sample the presence or level of reactivity between a sample obtained from the subject and at least one binding protein, at least one host cell that expresses the binding protein, or a population of host cells that express the binding protein, according to claim 56;
b) repeating step a) at a subsequent point in time; and
c) comparing the level of MAGEA1 or the cell of interest expressing MAGEA1 detected in steps a) and b) to monitor the progression of the disorder characterized by MAGEA1 expression in the subject, wherein an absent or reduced MAGEA1 level or the cell of interest expressing MAGEA1 detected in step b) compared to step a) indicates an inhibited progression of the disorder characterized by MAGEA1 expression in the subject and a presence or increased MAGEA1 level or the cell of interest expressing MAGEA1 detected in step b) compared to step a) indicates a progression of the disorder characterized by MAGEA1 expression in the subject,
optionally wherein
1) between the first point in time and the subsequent point in time, the subject has undergone treatment to treat the disorder characterized by MAGEA1 expression;
2) the level of reactivity is indicated by
A) the presence of binding; and/or
B) T cell activation and/or effector function, optionally wherein the T cell activation or effector function is T cell proliferation, killing, or cytokine release; and/or
3) the T cell binding, activation, and/or effector function is detected using fluorescence activated cell sorting (FACS), enzyme linked immunosorbent assay (ELISA), radioimmune assay (RIA), immunochemically, Western blot, or intracellular flow assay.

127. (canceled)

128. A method for predicting the clinical outcome of a subject afflicted with a disorder characterized by MAGEA1 expression comprising:
a) determining the presence or level of reactivity between a sample obtained from the subject and at least one binding protein, at least one host cell that expresses the binding protein, or a population of host cells that express the binding protein, according to claim 56; and
b) comparing the presence or level of reactivity to that from a control, wherein the control is obtained from a subject having a good clinical outcome;
wherein the absence or a reduced level of reactivity in the subject sample as compared to the control indicates that the subject has a good clinical outcome;
wherein the absence or a reduced level of reactivity in the subject sample as compared to the control indicates that the subject has a good clinical outcome,
optionally wherein
1) the level of reactivity is indicated by a) the presence of binding and/or b) T cell activation and/or effector function, optionally wherein the T cell activation or effector function is T cell proliferation, killing, or cytokine release; and/or
2) the T cell binding, activation, and/or effector function is detected using fluorescence activated cell sorting (FACS), enzyme linked immunosorbent assay (ELISA), radioimmune assay (RIA), immunochemically, Western blot, or intracellular flow assay.

129. A method of assessing the efficacy of a therapy for a disorder characterized by MAGEA1 expression comprising:
a) determining the presence or level of reactivity between a sample obtained from the subject and at least one binding protein, at least one host cell that expresses the binding protein, or a population of host cells that express the binding protein, according to claim 56, in a first sample obtained from the subject prior to providing at least a portion of the therapy for the disorder characterized by MAGEA1 expression to the subject, and
b) determining the presence or level of reactivity between a sample obtained from the subject and at least one binding protein, at least one host cell that expresses the binding protein, or a population of host cells that express the binding protein, according to claim 56, in a second sample obtained from the subject following provision of the therapy for the disorder characterized by MAGEA1 expression,
wherein the absence or a reduced level of reactivity in the second sample, relative to the first sample, is an indication that the therapy is efficacious for treating the disorder characterized by MAGEA1 expression in the subject, and wherein the presence or an increased level of reactivity in the second sample, relative to the first sample, is an indication that the therapy is not efficacious for treating the disorder characterized by MAGEA1 expression in the subject,
optionally wherein
1) the level of reactivity is indicated by
A) the presence of binding; and/or
B) T cell activation and/or effector function, optionally wherein the T cell activation or effector function is T cell proliferation, killing, or cytokine release; and/or
2) the T cell binding, activation, and/or effector function is detected using fluorescence activated cell sorting (FACS), enzyme linked immunosorbent assay (ELISA), radioimmune assay (RIA), immunochemically, Western blot, or intracellular flow assay.

130.-131. (canceled)

132. A method of preventing and/or treating a disorder characterized by MAGEA1 expression comprising contacting target cells expressing MAGEA1 with a therapeutically effective amount of a composition comprising cells expressing at least one binding protein according to claim 56, optionally wherein:
1) the composition is administered to a subject;
2) the cell is an allogeneic cell, syngeneic cell, or autologous cell;
3) the cell is a host cell that expresses the binding protein, or a population of host cells that express the binding protein, according to claim 56;
4) the target cell is a cancer cell expressing MAGEA1;
5) the composition
a) further comprises a pharmaceutically acceptable carrier;
b) induces an immune response against the target cell expressing MAGEA1 in the subject;
c) induces an antigen-specific T cell immune response against the target cell expressing MAGEA1 in the subject, optionally wherein the antigen-specific T cell immune response comprises at least one of a CD4⁺ helper T lymphocyte (Th) response and a CD8+ cytotoxic T lymphocyte (CTL) response;
6) at least one additional treatment is to be administered for the disorder characterized by MAGEA1 expression, optionally wherein the at least one additional treatment for the disorder characterized by MAGEA1 expression is to be administered concurrently or sequentially with the composition;
7) the disorder characterized by MAGEA1 expression is a cancer or relapse thereof, optionally wherein the cancer is selected from the group consisting of melanoma, head & neck cancer, lung cancer, cervical cancer, hepatocellular carcinoma, breast invasive carcinoma, and bladder urothelial carcinoma; and/or
8) the subject is an animal model of a disorder characterized by MAGEA1 expression and/or a mammal, optionally wherein the mammal is a human, a primate, or a rodent.

133.-142. (canceled)
